SEQUENCE LISTING (353 sequences)

SEQ ID NO: 1 - protein, 377 aa, Ovis aries
MCDEDETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVMSGGTTMYPGIADRMQKEITALAPSTMKIKIIAPPERKYSVWIGGSILASLSTFQQMWITKQEYDEAGPSIVHRKCF

SEQ ID NO: 2 - protein, 895 aa, Ovis aries
MMMVLQPEGLGTGEGPFAGGGRGGGEYMEQEEDWDRDLLLDPAWEKQQRKTFTAWCNSHLRKAGTQIENIEEDFRNGLKLMLLLEVISGERLPRPDKGKMRFHKIANVNKALDFIASKGVKLVSIGAEEIVDGNLKMTLGMIWTIILRFAIQDISVEETSAKEGLLLWCQRKTAPYRNVNVQNFHTSWKDGLALCALIHRHRPDLIDYAKLRKDDPIGNLNTAFEVAEKYLDIPKMLDAEDIVNTPKPDEKAIMTYVSCFYHAFAGAEQAETAANRICKVLAVNQENEKLMEEYEKLASELLEWIRRTVPWLENRVGEPSMSAMQRKLEDFRDYRRLHKPPRVQEKCQLEINFNTLQTKLRLSHRPAFMPSEGKLVSDIANAWRGLEQAEKGYEDWLLSEIRRLQRLQHLAEKFQQKASLHEAWTRGKEEMLSQRDYETASLQEVRALLRRHEAFESDLAAHQDRVEHIAALAQELNELDYHEAASVNSRCQAICDQWDNLGTLTQKRRDALERMEKLLETIDQLQLEFARRAAPFNNWLDGAVEDLQDVWLVHSVEETQSLVTAHDQFKATLPEADRERGAILGIQGEIQKICQTYGLRPSSTNPYIALTPQDINTKWDTVRKLVPSRDQMLQEELTRQQMNERLRRQFAAQANAIGPWIQGKVEEGGRLAAGMAGSLEEQMAGLRQQEQNIINYKSNIDRLEGDHQLLQESLVFDNKHTVYSMEHIRVGWEQLLTSIARTINEVENQVLTRDAKGLSQEQLNEFRASFNHFDRKRNGMMEPDDFRACLISMGYDLGEVEFARIMTMVDPNAAGVVTFQAFIDFMTRETAETDTAEQVVASFKILAGDKNYITAEELRRELPAEQAPQGARQGGRGSSWSPGLRGLLQCPLRGE

SEQ ID NO: 3 - protein, 359 aa, Cervus elaphus
MKSFILLFCLAQLCSCRSIPLDPIAGYKEPACDDPDTEQAALAAVDYINKHLPRGYKHTLNQIDSVKVWPRRPTGEVYDIEIDTLETTCHVLDPTPLANCSVRQQTEHAVEGDCDIHVLKQDGQFSVLFTKCDSSPDSAEDVRKLCPDCPLLAPLNDSRVVHAVEVALATFNAQSNGSYFQLVEISRAKFVPLPASVSVEFAVAATDCIAKDVVDPTKCNLLAEKQYGFCKGSVIQKAVGGEDVSVTCTVFQTQPVIPQPQPDGAEAGAPSAVPEAATPAPSAAGPPVASVVAGPSVVAVPLPLHRAHYDLRHTFSGVASVESASGEAFRVGKTPVGGQPSVPGGPVRLCPGRIRYFKI

SEQ ID NO: 4 - protein, 901 aa, Bos taurus
MMMVLQPEGLGTGEGPFAGGRGGGEYMEQEEDWDRDLLLDPAWEKQQRKTFTAWCNSHLRKAGTQIENIEEDFRNGLKLMLLLEVISGERLPRPDKGKMRFHKIANVNKALDFIASKGVKLVSIGAEGEEVAGGGRWGWGRGTTICTSQEQLIYQVETSAKEGLLLWCQRKTAPYRNVNVQNFHTSWKDGLALCALIHRHRPDLIDYAKLRKDDPIGNLNTAFEVAEKYLDIPKMLDAEDIVNTPKPDEKAIMTYVSCFYHAFAGAEQAETAANRICKVLAVNQENEKLMEEYEKLASELLEWIRRTVPWLENRVGEPSMSAMQRKLEDFRDYRRLHKPPRVQEKCQLEINFNTLQTKLRLSHRPAFMPSEGKLVSDIANAWRGLEQAEKGYEDWLLSEIRRLQRLQHLAEKFQQKASLHEAWTRGKEDMLSQRDYETASLQEVRALLRRHEAFESDLAAHQDRVEHIAALAQELNELDYHEAASVNSRCQAICDQWDNLGTLTQKRRDALERMEKLLETIDQLQLEFARRAAPFNNWLDGAVEDLQDVWLVHSVEETQSLVTAHDQFKATLPEADRERGAILGIQGEIQKICQTYGLRPSSTNPYITLTPQDINTKWDTVRKLVPSRDQMLQEELTRQQVNERLRRQFAAQANAIGPWIQGKVEEVGRLAAGMAGSLEEQMAGLRQQEQNIINYKSNIDRLEGDHQLLQESLVFDNKHTVYSMEHIRVGWEQLLTSIARTINEVENQVLTRDAKGLSQEQLNEFRASFNHFDRKRNGMMEPDDFRACLISMGYDLGEVEFARIMTMVDPNAAGVVTFQAFIDFMTRETAETDTAEQVVASFKILAGDKNYITAEELRRELPAEQAEYCIRRMAPYKGAGAPAGALDYVAFSSALYGESDL

SEQ ID NO: 5 - protein, 329 aa, Cervus elaphus
MADSEEVQRATALIEERLAQEEENEKLRGTTHQKLPMEMLVLEDEKHHRPESPSLQKVKGQERVRKTSLDLRREIIDVGGIQNLIQLRKKRKQKKREALATSQEPPPEPEEITGPVDEETFLKAAVEGKMKVIEKFLADGGSPDTCDQFRRTALHRASLEGHMEILEKLLERGATVDFQDRLDCTAMHWACRGGHLEVVRLLQSRGADTNVRDKLLSTPLHVAVRTGQVEIVEHFLSLGLDINAKDREGDSALHDAVRLNRYKIIKLLLLHGADMMSKNLAGKTPTDLVQLWQADTRHALENPEPGSEQNGLEGSAESGRETPQPVAAE

SEQ ID NO: 6 - protein, 341 aa, Anas platyrhynchos
MAMVSEFLKQAWFMDNQEQECIKSSKGGHSVQSYPNFDPSADVAALDKAITVKGVDEATIIDILTKRTNAQRQQIKAAYQQAKGKSLEEDLKKVLKGHLEDVAVALLKTPAQFDAEELRASMKGLGTDEDTLIEILASRNNREIREASRYYKEVLKRDLTQDIISDTSGDFQKALVALAKADRSEDPHVNDELADSDARALYEAGERRKGTDINVFVTVLTARSYPHLRRVFQKYTKYSKHDMNKVLDLELKGDIENCLTALVKCATSKPAFFAEKLHLAMKGLGTRHKQLIRIMVSRHEVDMNEIKAYYKSLYGISLRQAIMDELKGDYENILVALCGSD

SEQ ID NO: 7 - protein, 346 aa, Bos taurus
MAMVSEFLKQAWFIENEEQEYIKTVKGSKGGPGSAVSPYPTFNPSSDVEALHKAITVKGVDEATIIEILTKRNNAQRQQIKAAYLQEKGKPLDEVLKKALLGHLEEVVLALLKTPAQFDAEELRAAMKGLGTDEDTLNEILASRTNREIREINRVYREELKRDLAKDIASDTSGDYEKALLSLAKGDRSEELAVNDDLADSDARALYEAGERRKGTDVNVFITILTTRSYPHLRRVFQKYSKYSKHDMNKVLDLELKGDIEKCLTVIVKCATSQPMFFAEKLHQAMKGIGTRHKTLIRIMVSRSEIDMNDIKACYQKLYGISLCQAILDETKGDYEKILVALCGRD

SEQ ID NO: 8 - protein, 346 aa, Cervus elaphus
MAMVSEFLKQAWFIENEEQEYIKTVKGYKGGPGSAVSPYPTFNPSSDVEALHKAITVRGVDEATIIEILTKRNNAQRQQIKAAYLQEKGKPLDEVLKKALVGHLEEVVLALLKTPAQFDAEELRAAMKGLGTDEDTLNEILASRTNGEIREINRVYREELKRDLAKDIASDTSGDYEKALLSLAKGDRSEELAVNDDLADSDARALYEAGERRKGTDVNVFTTILTTRSYPHLRRVFQKYSKYSKHDMNKVLDLELKGDIEKCLTVIVKCATNKPMFFAEKLHQAMKGIGTRHKTLIRIMVSRSEIDMNDIKACYQKLYGISLCQAILDETKGDYEKILVALCGGN

SEQ ID NO: 9 - protein, 130 aa, Gallus gallus
MAMVSEFLKQAWFMDNQEQECIKSSKGGSSVQSRPNFDPSADVSALDKAITVKGVDEATIIDILTKRTNAQRQQIKAAYQQAKGKSLEEDLKKVLKSHLEDVVVALLKTPAQFDAEELRASMKGLGTDRR

SEQ ID NO: 10 - protein, 342 aa, Gallus gallus
MAMVSEFLKQAWFMDNQEQECIKSSKGGSSVQSRPNFDPSADVSALDKAITVKGVDEATIIDILTKRTNAQRQQIKAAYQQAKGKSLEEDLKKVLKSHLEDVVVALLKTPAQFDAEELRASMKGLGTDEDTLIEILASRNNREIREASRYYREVLKKDLTQDIISDTSGDFQKALVILAKGDRCEDPHVNDDLADNDARALYEAGEKRKGTDVNVFITILTSRSYPHLRRVFQKYAKYSKHDMNKVLDLELKGDIENCLTALVKCATSKPAFFAEKLHLAMKGSGTRHKQLIRIMVSRHEIDLNEIKAYYKSLYGISLRQAIMDELKGDYETILVALCGSDK

SEQ ID NO: 11 - protein, 260 aa, Meleagris gallopavo
MSQQARRLCRLNRELWLDLKNKRKVYGLWKSGRATYEDYRQGLGTDEDTLIEILASRNNREIREASRYYKEVLKRDLIQDIISDTSGDFQKALVTLAKGDRSEDPHVNDDLADNDARALYEAGEKRKGTDINVFITVLTSRSYLHLRRVFQKYAKYSKHDMNKVLDLELKGDIENCLTALVKCATSKPAFFAEKLHLAMKGSGTRHKQLIRIMVSRHEVDLNEIKAYYKSLYGISLRQAIMDELKGDYETILVALCGSDK

SEQ ID NO: 12 - protein, 346 aa, Oryctolagus cuniculus
MAMVSEFLKQAWFIDNEEQDYINTVKTYKGGPGSAVSPYPAFNPSSDVAALHQAIMVKGVDEATIIDILTKRNNAQRQQIKAAYLQEKGKPLDEVLKKALTGHLEEVVLALLKTPAQFDADELRAAMKGLGTDEDTLIEILASRNNKEIREINRVYREELKRDLAKDIASDTSGDFQKALLSLAKGDRSEDFGVNEDLADTDARALYEAGERRKGADVNVFTTILTTRSYLHLRRVFQKYSKYSQHDMNKVLDLELKGDIEKCLTAIVQCATCKPAYFAEKLYQAMKGAGTRHKALIRIMVSRSEVDMNDIKAFYQKKYGVSLCQAILDETKGDYEKILVALCGGN

SEQ ID NO: 13 - protein, 346 aa, Ovis aries
MAMVSEFLKQAWFIENEEQEYIKTVKGSKGGPGSAVSPYPTFNPSSDVEALHKAITVKGVDEATIIEILTKRNNAQRQQIKAAYLQEKGKPLDEVLKKALLGHLEEVVLALLKTPAQFDAEELRAAMKGLGTDEDTLNEILASRTNGEIREINRVYREELKRDLAKDIASDTSGDYEKALLSLAKGDRSEELAVNDDLADSDARALYEAGERRKGTDVNVFTTILTTRSYPHLRRVFQKYSKYSKHDMNKVLDLELKGDIEKCLTVIVKCATSKPMFFAEKLHQAMKGVGTRHKTLIRIMVSRSEIDMNDIKACYQKLYGISLCQAILDETKGDYEKILVALCEGN

SEQ ID NO: 14 - protein, 339 aa, Salmo salar
MSFIAAFLQQTVYLGLPDDSTLPNQGTVTPDPHFSVDGDVGILDKAIKAKGVDENTIIDVLVRRSNAQRQQIKATYEKASGKPLETALKSALKGDLEDVVLALLKTPAQYDAQQLKLAMKGLGTDEDTLIEILASRTNKEIREIKKVYKGEYKKELEDDIKSDTGADFRNALLSLCKATRNEDTMVNQELADSDARALYEAGEKRKGTDCSVFIDILTTRSAPQLRQAFERYSKYSKVDVAKAIDLELKGDIENCLTAVVKCAGSKPAFFAERLNLAMKGKGTRTNILTRVMVSRSEIDLARIKQEYKKTFGKTLSQEILDDTKGDYEKILLALCGSDN

SEQ ID NO: 15 - protein, 346 aa, Sus scrofa
MAMVSEFLKQAWFIDNEEQEYIKTVKGSKGGPGSAVSPYPTFNPSSDVEASHKAITVKGVDEATIIEIHTKRTNAQRQQIKAAYLQEKGKPLDEALKKALTGHLEEVALALLKTPAQFDADELRAAMKGLGTDEDTLNEILASRTNREIREINRVYKEELKRDLAKDITSDTSGDYQKALLSLAKGDRSEDLAINDDLADTDARALYEAGERRKGTDLNVFITILTTRSYLHLRRVFQKYSKYSKHDMNKVLDLELKGDIENCLTVVVKCATSKPMFFAEKLHQAMKGNGTRHKTLIRIMVSRSEIDMNDIKACYQKLYGISLCQAILDETKGDYEKILVALCGGD

SEQ ID NO: 16 - protein, 304 aa, Anas platyrhynchos
MDAQVLGGALQGISCEKDVLIDILTQRCNSQRLMIAEAYRDMYGRDLITDLKENLSHHFKEVMVGLMYPPASYDAHELWHALKGVDTEENCLIDILASRSNMEIFQMKEAYLMQYNNDLQQDIDSETSGHFRDTLMNLAQGTRMEGYADPSSAAQDAMILWEACQRKTGEHKDMLQMILCNKSYQQLWMVFQEFQNISGQDLVDAINECYDGYFQELLVAIVLCVRDKPSYFAYRLYNAIHEFGFHNKTVIRILIARSEIDLMNIRQRYKERYGKSLFHDIKHFASGHYERALLAICAGDAEDY

SEQ ID NO: 17 - protein, 324 aa, Bos taurus
MFCGDYVQGTIFPAPNFNPVLDAELLGGALQGFGCDKDLLIDILTQRCNTQRLLIAEAYLGAFSRDLVGDLKEKLSNHFKDVMVGLMYPPPSYDAHELWHAMKGAGTDENCLIDILASRTNGEIFQMREAYYLQYSRDLQEDIYSETSGHFRDMLVSLVQGTREEGYTDPATATQDAMVLWEACQQRTGEHKTLLQLMLCNKSYQQLCLVFQEFQNISGQDIVDAINECYDGYFRELLVATVLCIRDKPAYFAYRLYSAIHDFGFHNKTVIRILIARSEIDLMTIRKRYKERYGKSLFHDIKNFASGHYEKALLAICAGDADDY

SEQ ID NO: 18 - protein, 324 aa, Cervus elaphus
MFCGDYVQGTIFPAPNFNPVMDAELLGGALQGFGCDKDLLIDILTQRCNTQRLLIAEAYLGAFGRDLVGDLKEKLSNHFKDVMVGLMYPPPSYDAHELWHAMKGAGTDENCLIDILASRTDREIFQMREAYYLQYSRDLQEDIYSETSGHFRDMLVSLVQGTREEGYADPATATQDAMVLWQACQQRTGEHKTLLQVMLCNKSCQQLGLVFQEFQNISGQDIVDAINECYEGYFRELLVAIVLCVRDKPAYFAYRLYTAIHDLGFHNKTVIRILISRSEIDLMSIRKRYKERYGKSLFHDIKNFASGHYEKALLAICAGDTDDY

SEQ ID NO: 19 - protein, 325 aa, Gallus gallus
MYCGDYQIQGTIFPAPNFNPIMDAQLLGGALQGISCEKDVLIDILTQRCNSQRLMIAEAYRDMYGRDLITDLKENLSHHFKEVMVGLMYPPAAYDAHELWHALKGVNTEEKCLIDILASRSNMEIFQMKEAYLTQYNSDLQQDIDSETSGHFRDTLMNLAQGTRMEGYADPSTAAQDAMILWEACQQKTGEHKNMLQMILCNRSYQQLWMVFQEFQNISGQDLVDAINDCYDGYFQELLVAIVLCVRDKASYFAYRLHNAIHDFGFHNKTVIRILIARSEIDLMTIRQRYKERYGKSLFHDIKHFASGHYESALLAICAGDAEDY

SEQ ID NO: 20 - protein, 324 aa, Gallus gallus
MYCGDYIQGTIFPAPNFNPIMDAQLLGGALQGISCEKDVLIDILTQRCNSQRLMIAEAYRDMYGRDLITDLKENLSHHFKEVMVGLMYPPAAYDAHELWHALKGVNTEEKCLIDILASRSNMEIFQMKEAYLTQYNSDLQQDIDSETSGHFRDTLMNLAQGTRMEGYADPSTAAQDAMILWEACQQKTGEHKNMLQMILCNRSYQQLWMVFQEFQNISGQDLVDAINDCYDGYFQELLVAIVLCVRDKASYFAYRLHNAIHDFGFHNKTVIRILIARSEIDLMTIRQRYKERYGKSLFHDIKHFASGHYESALLAICAGDAEDY

SEQ ID NO: 21 - protein, 324 aa, Meleagris gallopavo
MYCGDYIQGTIFPAPNFNPIMDAQLLGGALQGISCEKDVLIDILTQRCNSQRLMIAEAYRDMYGRDLMTDLKENLSHHFKEVMVGLMYPPASYDAHELWHALKGVNTEEKCLIDILASRSNMEIFQMKEAYLTQYNSDLQQDLDSETSGHFRDTLMNLAQGTRMEGYADPSTAAQDAMILWEACQQKTGEHKNMLQMILCNRSYQQLWMVFQEFQSISGQDLVDAINDCYDGYFQELLVAIVLCVRDKPSYFAYRLYNAIHDFGFHNKTVIRILIARSEIDLMTIRQRYKERYGKSLFHDIKHFASGHYESALLAICAGDVEDY

SEQ ID NO: 22 - protein, 313 aa, Meleagris gallopavo
MYCGDYIQGTIFPAPNFNPIMDAQLLGGALQGISCEKDVLIDILTQRCNSQRLMIAEAYRDMYGRDLMTDLKENLSHHFKEVMVGLMYPPASYDAHELWHALKGVNTEEKCLIDILASRSNMEIFQMKEAYLTQYNSDLQQDLDSETSGHFRDTLMNLAQGTRMEGYADPSTAAQDAMILWEACQQKTGEHKNMLQMILCNRSYQQLWMVFQEFQSISGQDLVDAINDCYDGYFQELLVAIVLCVRDKPSYFAYRLYNAIHDFGFHNKTVIRILIARSEIDLMTIRQRYKERYGKSLFHDIKIFHPDIDLRFV

SEQ ID NO: 23 - protein, 316 aa, Oryctolagus cuniculus
MHVSCKSCLNALATETHISMRAGEPECDKDLLIDILTQRCNAQRLMIAEAYQSMYGRDLIGMLKEQLSDHFKDVMVGLMYPPPSYDAHELWHAMKGAGTDENCLIDILASRTNGEIFQMREAYSLQYNNNLQEDIQSETSGHFRDTLMNLVQATRQEGYTDPAMAAQDAMVLWEACQQKTGAHKTMLQMILCNKSYQQLWLVFQEFQNISGQDIVDAINDCYDGYFQQLLVAIVLCVRDKPAYFAYRLHSAIHDFGFHNKTVIRILIARSEIDLMTIRTRYKERYGKSLFHDIRHFASGHYKKALLAICAGDMEDC

SEQ ID NO: 24 - protein, 324 aa, Ovis aries
MFCGDYVQGTIFPAPNFNPVLDAELLGGALQGFGCDKDLLIDILTQRCNTQRLLIAEAYLGAFSRDLVGDLKEKLSNHFKDVMVGLMYPPPSYDAHELWHAMKGAGTDESCLIDILASRTNGEIFQMREAYYLQYSRDLQEDIYSETSGHFRDMLVSLVQGTREEGYTDPATATQDAMVLWEACQQRTGEHKTLLQLMLCNKSYQQLCLVFQEFQSISGQDIVDAINECYDGYFRELLVAVVLCIRDKPAYFAYRLYSAIHDFGFHNKTVIRILIARSEIDLMTVRKRYRERYGKSLFHDIKDFASGHYEKALLAICAGDADDY

SEQ ID NO: 25 - protein, 324 aa, Sus scrofa
MFCGDYVQGTIVPAPNFNPMLDAQMLGGALQGFGCDKDLLIDILTQRCNAQRLVIAEAYQGAFCRDLVSDLKEKLSDHFKDVMVGLMYPPPSYDAHELWHAMKGAGTDENCLIDILASRTNGEIFQMREAYYLQYSRDLQEDIYAETSGHFRDMLVNLVQGTREEGYTDPAMAAQDAMVLWEACQQRTGEHKTLLQMILCNKSYQQLWLVFQEFQNISGQDIVDAINECYDGYFQELLVAVVLCVRDKPAYFAYRLYSAIHDFGFHNRTVIRILIARSEIDLMTIRKRYKERYGKSLFHDIRNFASGHYEKALLAICAGDTDEY

SEQ ID NO: 26 - protein, 511 aa, Anas platyrhynchos
MSYSGYPPAAPGYPPGAPGYPPPAPGGSPWGGAAYPPSNPPSNPPPIGLENVAGYANQFNPNYMAGMASNMAGTFGGTNVPQGMYPPAPGAFPPVPPGGFGQPPSGQQPSYGMYPPPGGNPPSGMPSYPGYPGGPVPGQPTPPPGQQPVGYPGQQPATYPGQQPMPSYPPGQTVNPSMPSFPGPSGPTVSPGAYGNRGTITDAPGFDPLRDAEVLRKAMKGFGTDEQAIIDCLGSRSNKQRQQIILSFKTAYGKDLIKDLKSELSGNFERTILAMMKTPVMFDAYEIKEAIKGVGTDENCLIEILASRSNEHIQELNRVYKAEFKKTLEEAIKSDTSGHFQRLLISLSQGNRDESTAVDMSLVQKDVQELYAAGENRLGTDESKFNAILCARSRAHLRAVFSEYQRMCNRDIEKSICSEMSGDLEKGMLAVVKCLKNTPAFFAERLRNAMKGAGTKDRTLIRIMVSRSEVDLLDIRAEYKRMYGKSLYTDITGDTSGDYRKILLKLCGGND

SEQ ID NO: 27 - protein, 505 aa, Bos taurus
MSYPGYPPPAGGYPPGAPGVPELESHAGGPQGLFAAMDRAVSDGPAMMLAAVLLVRATAANMSGTFGGANVPNLYPGAPGGGYPPVPPGGFGQPPPAQQPVPSYGMYPPPGGNPTSGMPSYPPYPGAPVPGQPMLPPGQQPPGVYPGQPPMTYPGQSPVPPPGQQPVPSYPGYSGSGTVTPAVSPAQFGNRGTITDASGFDPLRDAEVLRKAMKGFGTDEQAIIDCLGSRSNKQRQQILLSFKTAYGKDLIKDLKSELSGNFEKTILALMKTPVLFDAYEIKEAIKGAGTDEACLIEILASRSNEHIRELNRVYKTEFKKTLEEAIRSDTSGHFQRLLISLSQGNRDESTNVDMTLVQRDVQELYAAGENRLGTDESKFNAILCSRSRAHLVAVFNEYQRMTGRDIEKSICREMSGDLEQGMLAVVKCLKNTPAFFAERLNKAMRGAGTKDRTLIRIMVSRSEIDLLDIRAEYKRLYGKSLYHDITGDTSGDYRKILLKICGGND

SEQ ID NO: 28 - protein, 503 aa, Cervus elaphus
MSYPGYPPPAGGYPPAAPGGGAWGGAGYPPPTMPPIGLDNVANYAGQFNQDYLSGVAANMSGTFGGANVPNLYPGAPGAGYPPVPPGSFGQPPPAQQPVPSYGMYPPPGGNPPSGMPSYPPYPGAPVPGQPMLPPGQQPPGAYPGQPPMTYPGQSPMPPPGQQPVPSYPGYSGSGTVTPAVSPAQFGNRGTITDASGFDPLRDAEVLRKAMKGFGTDEQAIIDCLGSRSNKQRQQILLSFKTAYGKDLIKDLKSELSGNFEKTILALMKTPVLFDAYEIKEAIKGAGTDEACLIEILASRSNEHIRELNRLYKTEFKKTLEEAIRSDTSGHFQRLLISLSQGNRDESTNVDMTLVQRDVQELYAAGENRLGTDESKFNAILCSRSRAHLVAVFNEYQRMTGRDIEKSICREMSGDLEQGMLAVVKCLKNTPAFFAERLNKAMRGAGTKDRTLIRIMVSRSEIDLLDIRAEYKRLYGKSLYHDITGDTSGDYRKILLKICGGND

SEQ ID NO: 29 - protein, 492 aa, Gallus gallus
MSHPGYPPAGGYPPAAPGGSPWGGAAYPPSNPPPIGLENVAGYANQFNPSYMAGMASNMAGTFGGSNVPQGIYPSAPGGYPPVPAGGFGQPPPGQQPSYGGYPPSGGNAPGFPGGPALGQPTLPPGQPPMGLPGQPPAPYPGQQPMPTYPPVPAVNPSMPSYPGPTGPTVSPGAYGNRGTITDALGFDPLKDAEVLRKAMKGLGTDEQAIIDCLGSRSNKQRQQIILSFKTAYGKDLIKDLKSELSGNFEKTILAMMKTPVMFDAYEIKEAIKGIGTDENCLIEILASRSNEHIQELNRVYKAEFKKTLEEAIRSDTSGHFQRLLISLSQGNRDESTNVDMSLVQKDVQELYAAGENRLGTDESKFNAILCARSRAHLRAVFSEYQRMCNRDIENSICREMSGDLEKGMLAVVKCLKNTPAFFAERLRNAMKGAGTKDRTLIRIMVSRSEVDLLDIRAEYKRMYGKSLYADITGDTSGDYRKILLKLCGGND

SEQ ID NO: 30 - protein, 482 aa, Meleagris gallopavo
MSYPGYPPAGGYPPAPPGGSPWGSAVYPPSNPPPIGLENVAGYANQFNPSYMAGMASNMAGTFGGSNVPQGIYPSAPGGYPPVPPGGFGQPPSGQQPSYGGYPPPGGNIPGQPTLPPGQLPMGSPGQPPAPYPGQQPMPSYPPAVNPSVPSYPGPTGPAVSPGAYGNRGTITDAPGFDPLKDAEVLRKAMKGLGTDEQAIIDCLGSRSNKQRQQIILSFKTAYGKDLIKDLKSELSGNFEKTILAMMKTPIMFDAYEIKEAVKGIGTDENCLIEILASRSNEHIQELNRVYKAEFKKTLEEAIRSDTSGHFQRLLISLAQGNRDESTNVDMSLVQKDVQELYAAGENRLGTDESKFNAILCARSRAHLRAVFSEYQRMCNRDIEKSICREMSGDLEKGMLAVVKCLKNTPAFFAERLRNAMKGAGTKDRTLIRIMVSRSEVDLLDIRAEYKRLYGKSLYADITGDTSGDYRKILLKLCGGND

SEQ ID NO: 31 - protein, 503 aa, Oryctolagus cuniculus
MSYPGYPPPPGGYPPAPGGGAWGGAGYPPPSMPPIGLDNVANYAGQFNQDYLSGMAANMSGTFGGANVPPNLYPGAPGGGYPPVPPGGFGQPPPTQPSVPPYGVYPPPGGNPPSGVPSYPPFPGAPVPGQPMPPPGHQPPGPYPGQLPVTYPGQSPVPPPGQQPMPSYPGYPGSGTVTPAVPPVQFGNRGTITDASGFDPLRDAEVLRKAMKGFGTDEQAIIDCLGSRSNKQRQQILLSFKTAYGKDLIKDLKSELSGNFEKTILALMKTPILFDAYEIKEAIKGAGTDEACLIEILASRSNEHIRELNKAYKTEFKKTLEEAIRSDTSGHFQRLLISLSQGNRDESTNVDMSLVQRDVQELYAAGENRLGTDESKFNAVLCSRSRAHLVAVFNEYQRMTGRDIEKSICREMSGDLEQGMLAVVKCLKNTPAFFAERLNRAMRGAGTKDRTLIRIMVSRSEIDLLDIRAEYKRMYGKSLYHDISGDTSGDYRKILLKICGGND

SEQ ID NO: 32 - protein, 503 aa, Ovis aries
MSYPGYPPPAGGYPPAAPGGGAWGGAGYPPPTMPPIGLDNVANYAGQFNQDYLSGVAANMSGTFGGASVPNLYPGTPGGGYPPVPPGSFGQPPPAQQPVPSYGMYPPPGGNPPSGMPSYPPYPGAPVPGQPMLPPGQQPPGAYPGQPPMTFPGQSPVPPPGQHPAPSYPGYSGSGTVTPAVSPAQFGNRGTITDASGFDPLRDAEVLRKAMKGFGTDEQAIIDCLGSRSNKQRQQILLSFKTAYGKDLIKDLKSELSGNFEKTILALMKTPVLFDACEIKEAIKGAGTDEACLIEILASRSNEHIRELNRLYKTEFKKTLEEAIRSDTSGHFQRLLISLSQGNRDESTNVDMTLVQRDVQELYAAGENRLGTDESKFNAILCSRSRAHLVAVFNEYQRMTGRDIEKSICREMSGDLEQGMLAVVKCLKNTPAFFAERLNKAMRGAGTKDRTLIRIMVSRSEIDLLDIRAEYKRLYGKSLYHDITGDTSGDYRKILLKICGGND

SEQ ID NO: 33 - protein, 293 aa, Ovis aries
MKGFGTDEQAIIDCLGSRSNKQRQQILLSFKTAYGKDLIKDLKSELSGNFEKTILALMKTPVLFDACEIKEAIKGAGTDEACLIEILASRSNEHIRELNRLYKTEFKKTLEEAIRSDTSGHFQRLLISLSQGNRDESTNVDMTLVQRDVQELYAAGENRLGTDESKFNAILCSRSRAHLVAVFNEYQRMTGRDIEKSICREMSGDLEQGMLAVVKCLKNTPAFFAERLNKAMRGAGTKDRTLIRIMVSRSEIDLLDIRAEYKRLYGKSLYHDITGDTSGDYRKILLKICGGND

SEQ ID NO: 34 - protein, 236 aa, Ovis aries
MKTPVLFDACEIKEAIKGAGTDEACLIEILASRSNEHIRELNRLYKTEFKKTLEEAIRSDTSGHFQRLLISLSQGNRDESTNVDMTLVQRDVQELYAAGENRLGTDESKFNAILCSRSRAHLVAVFNEYQRMTGRDIEKSICREMSGDLEQGMLAVVKCLKNTPAFFAERLNKAMRGAGTKDRTLIRIMVSRSEIDLLDIRAEYKRLYGKSLYHDITGDTSGDYRKILLKICGGND

SEQ ID NO: 35 - protein, 530 aa, Salmo salar
MSYPGYPPSGGYAPQPGAYPPQAGGYPPQAGGYPPQAGGYPPQAGGYPPQAGGYPPQAGGYPSQQAGGYPPQAGGYPSQQAGGYPPQAGGYPSQQAGGYPQPGGGFPQGGGFPPQAGGYPSMPPAQGGWGGAPAGGMPGGGMPQGYPGGPAPGQPQGYPGGPAPGQPQGYPGGPAPGQQPMPGYPGGAPAPNPSMPGYGGGVPATPYSPAINRGYRGVIKDYPGADPLRDVEVLRKAMKGFGTDEAAIIEILGSRSNKQRVPMVAAYKTTYGKDLFHDLKSELTGNFEKLAIAMLQTSTRFDASQLKEAIVGAGTDEACLIEILSSRSNAEIREINQIYKNEYGKKLEDAIINDTSGHFRRLLVSLCQGNRDEREQVDINMAKQDAQKLYAAGENKVGTDESQFNAIMCSRSKPHLRAVFNEYQQMSGRDIVKSICREMSGNVEDGMVAVVKCIRNTPEYFAERLHKSMAGAGTKDRTLIRVMVTRSEVDMLDIREAYQKAYGKSLYTAISGDTSGDYKKLLLKLCGGSD

SEQ ID NO: 36 - protein, 554 aa, Salmo salar
MSYPGYPPQAGGYPPQAGGYPPQAGGYPPQAGGYPPQAGGYPPQAGGYPPQAGGYPPQAGGYPSQAGGYPPQAGGYPPQPGAGGYPAMPPAGSWGGGAGFPTIGVDNLSNPGFNAGNLPGMANQISSSMGGFNPNPAMFTPGGYPQHSPQPPNQQPHGLYPQPGGQMPQTQGPGMGYPGQPMPGYPQAPSPNPSMPGYGGGPAPNQPMPGYPRAPSPNPSMPGYGGGAMPVAPAINRGFRGSIKDFPGADPLRDVEVLRKAMKGFGTDEQAIIDLLGSRSNRQRVPMLMAFKTSYGKDLVKDLKSELSGNFEKLVLAMLKTPSQLDAYELKEAIKGAGTDEACLIEILSSRSNAEIRELNQVYKTEYKKSLEDAISGDTSGHFRRLLISLAQGNRDERETVDISLAKQDAQALYAAGENKVGTDESKFNAILCSRSKSHLRAVFHEYQQMCGRDLEKSIDREMSGDLESGMVAVVKCIKNTPAYFAERLYKSMKGAGTKDKTLIRIMVTRSEVDMLDIRQEYVKNYGKSLYTDISGDTSGDYKKLLLKLCGGSD

SEQ ID NO: 37 - protein, 502 aa, Sus scrofa
MSYPGYPPPTGGYPPAAPGGAWGGAAYPPPSMPPIGLDNVANYAGQFNQDYLSGMAANMSGTFGGANVPNLYPGAPGGGYPPVPPGGFGQPPSAQQPVPPYGMYPPPGGNPPSGMPTYPPYPGAPVPGQPMPPPGQQPPGAYPGQPPMPYAGQSPMPPPGQQPMPSYPGYSGSGTVTPAVPPAQFGNRGTIADAPGFDPLRDAEVLRKAMKGFGTDEQAIIDCLGSRSNKQRQQILLSFKTAYGKDLIKDLKSELSGNFEKTILALMKTPILFDAYEIKEAIKGAGTDEACLIEILASRSNEHIRELSRAYKTEFKKTLEDAIRSDTSGHFQRLLISLSQGNRDESTNVDMALVQRDVQELYAAGENRLGTDESKFNAILCSRSRAHLVAVFNEYQRMTGRDIEKSICREMSGDLEEGMLAVVKCLKNTPAFFAERLNKAMRGAGTKDRTLIRIMVSRSEIDLLDIRAEYKRLYGKSLYHDITGDTSGDYRKILLKICGGND

SEQ ID NO: 38 - protein, 372 aa, Sus scrofa
MPPPGQQPPGAYPGQPPMPYAGQSPMPPPGQQPMPSYPGYSGSGTVTPAVPPAQFGNRGTIADAPGFDPLRDAEVLRKAMKGFGTDEQAIIDCLGSRSNKQRQQILLSFKTAYGKDLIKDLKSELSGNFEKTILALMKTPILFDAYEIKEAIKGAGTDEACLIEILASRSNEHIRELSRAYKTEFKKTLEDAIRSDTSGHFQRLLISLSQGNRDESTNVDMALVQRDVQELYAAGENRLGTDESKFNAILCSRSRAHLVAVFNEYQRMTGRDIEKSICREMSGDLEEGMLAVVKCLKNTPAFFAERLNKAMRGAGTKDRTLIRIMVSRSEIDLLDIRAEYKRLYGKSLYHDITGDTSGDYRKILLKICGGND

SEQ ID NO: 39 - protein, 343 aa, Anas platyrhynchos
MGSSHSKSYNLPDDVRKPTVGTTNLSADKPSTEKRHHHHGFDAERDAKKIHSACKGAGTDEKKIIEVLSSRTAEQRQQIKQKYNALYGKDMAEVLKGDLSGNFEKAVLALLDLPCEYEARELRKAMKGAGTDESLLIEILCTRSNKEIINIKEAYKRLFDRDLESDVKSDTSGSLRKVLVTVLEATRDESQQVNTELAEQDATDLYKAGEGRWGTEELAFNVVLAKRSYSQLRATFQAYEKVCGKDIEESIKSETSGDLEKAYLTLVSCAKDCPGYFATLLHKSMKGAGSDEETLIRVLVTRSETDLPEIKGKFQQMYKKSLTEAVRSDTSGDFRKLLLAILH

SEQ ID NO: 40 - protein, 332 aa, Anas platyrhynchos
MGSSHSKSYNLPDDVRKPTSTEKRHHHHGFDAERDAKKIHSACKGAGTDEKKIIEVLSSRTAEQRQQIKQKYNALYGKDMAEVLKGDLSGNFEKAVLALLDLPCEYEARELRKAMKGAGTDESLLIEILCTRSNKEIINIKEAYKRLFDRDLESDVKSDTSGSLRKVLVTVLEATRDESQQVNTELAEQDATDLYKAGEGRWGTEELAFNVVLAKRSYSQLRATFQAYEKVCGKDIEESIKSETSGDLEKAYLTLVSCAKDCPGYFATLLHKSMKGAGSDEETLIRVLVTRSETDLPEIKGKFQQMYKKSLTEAVRSDTSGDFRKLLLAILH

SEQ ID NO: 41 - protein, 318 aa, Anas platyrhynchos
MGSSHSTEKRHHHHGFDAERDAKKIHSACKGAGTDEKKIIEVLSSRTAEQRQQIKQKYNALYGKDMAEVLKGDLSGNFEKAVLALLDLPCEYEARELRKAMKGAGTDESLLIEILCTRSNKEIINIKEAYKRLFDRDLESDVKSDTSGSLRKVLVTVLEATRDESQQVNTELAEQDATDLYKAGEGRWGTEELAFNVVLAKRSYSQLRATFQAYEKVCGKDIEESIKSETSGDLEKAYLTLVSCAKDCPGYFATLLHKSMKGAGSDEETLIRVLVTRSETDLPEIKGKFQQMYKKSLTEAVRSDTSGDFRKLLLAILH

SEQ ID NO: 42 - protein, 81 aa, Bos taurus
MGNRHSQSYSLPEGSQHLPKGQALPSADLQPLSHPSGSGEPESHQPVMDWIWPPSIVCQLLDWKSVKNILLPDKDWLLTRI

SEQ ID NO: 43 - protein, 371 aa, Cervus elaphus
MGNRHSQSYSLPEGSQQLPQGQALPSADLQPLSHPSGSGEPESHQPAKGKSHQGLGAGRDNKAKSHQRFDADQDAKKLNKACKGMGTDEAAIIEILSSRTSNERQQIKQKYKTAYGKDLEEVLKSELSGNFEKTALALLDRPEEYAARQLQKAMKGLGTDEAVLIEVLCTRTNKEIIAIKEAYQRLFNKSLESDVKGDTSGSLKGILVSLLQANRDEGDNVDKDLAGQDAKELYDAGEGRWGTEELAFNELLAKRSHKQLRATFQAYQTLIGKDIEEAIEAETSGDLQKAYLTLVRSARDLQGYFADRLYKSMKGAGTDEKTLIDIIVTRAEVDLQGIKARFQEKYQKSLSDMVRSDTSGDFQKLLVALLH

SEQ ID NO: 44 - protein, 358 aa, Cervus elaphus
MRKSLLPKLTGLIVPENREAHSLWASESSWLQEAKGKSHQGLGAGRDNKAKSHQRFDADQDAKKLNKACKGMGTDEAAIIEILSSRTSNERQQIKQKYKTAYGKDLEEVLKSELSGNFEKTALALLDRPEEYAARQLQKAMKGLGTDEAVLIEVLCTRTNKEIIAIKEAYQRLFNKSLESDVKGDTSGSLKGILVSLLQANRDEGDNVDKDLAGQDAKELYDAGEGRWGTEELAFNELLAKRSHKQLRATFQAYQTLIGKDIEEAIEAETSGDLQKAYLTLVRSARDLQGYFADRLYKSMKGAGTDEKTLIDIIVTRAEVDLQGIKARFQEKYQKSLSDMVRSDTSGDFQKLLVALLH

SEQ ID NO: 45 - protein, 330 aa, Cervus elaphus
MGNRHAKGKSHQGLGAGRDNKAKSHQRFDADQDAKKLNKACKGMGTDEAAIIEILSSRTSNERQQIKQKYKTAYGKDLEEVLKSELSGNFEKTALALLDRPEEYAARQLQKAMKGLGTDEAVLIEVLCTRTNKEIIAIKEAYQRLFNKSLESDVKGDTSGSLKGILVSLLQANRDEGDNVDKDLAGQDAKELYDAGEGRWGTEELAFNELLAKRSHKQLRATFQAYQTLIGKDIEEAIEAETSGDLQKAYLTLVRSARDLQGYFADRLYKSMKGAGTDEKTLIDIIVTRAEVDLQGIKARFQEKYQKSLSDMVRSDTSGDFQKLLVALLH

SEQ ID NO: 46 - protein, 316 aa, Gallus gallus
MGGSHSTDKRRHHGFDAERDAKKLHSACKGAGTDEKKIIEVLSSRTSEQRQQIKQKYKALYGKDLEEVLKGDLSGSFEKAVLALLDLPCEYKARELHKAMKGAGTDESLLIEILCTQNNQEIINTKEAYKRLFAKDLESDVKGDSGSLRKILVTVLEATRDENQQVNTELAEQDASDLYKAGEGRWGTEELAFNVVLAKRSYSQLRATFQAYEKMCGKDIEESIKSETSGDLEKAYLTLVSCAKDCPGYFATLLHESMKGAGTDEDTLIRILVTRAESDLPAIKGKFQEMYKKSLTEAVRSDTSGDFRKLLLAILH

SEQ ID NO: 47 - protein, 342 aa, Gallus gallus
MGGSHSKSYNLPEDKREPTVGTTSLSTDKPSTDKRRHHGFDAERDAKKLHSACKGAGTDEKKIIEVLSSRTSEQRQQIKQKYKALYGKDLEEVLKGDLSGSFEKAVLALLDLPCEYKARELHKAMKGAGTDESLLIEILCTQNNQEIINTKEAYKRLFAKDLESDVKGDTSGSLRKILVTVLEATRDENQQVNTELAEQDASDLYKAGEGRWGTEELAFNVVLAKRSYSQLRATFQAYEKMCGKDIEESIKSETSGDLEKAYLTLVSCAKDCPGYFATLLHESMKGAGTDEDTLIRILVTRAESDLPAIKGKFQEMYKKSLTEAVRSDTSGDFRKLLLAILH

SEQ ID NO: 48 - protein, 342 aa, Meleagris gallopavo
MGSSHSKSYNLPEDKREPTVGTTSLSTDEPSTDKRRHHGFDADRDAKKLHSACKGAGTDEKKIIEVLSSRTSEQRQQIKQKYKALYGKDLEEVLKGDLSGSFEKAVLALLDLPCEYKARELHKAMKGAGTDESLLIEILCTQNNKEITSIKEAYKRLFDKDLESDVKGDTSGSLRKILVAVLEATRDENQQVNIELAEQDASDLYKAGEGRWGTEELAFNVVLAKRSYSQLRATFQAYEKVCGKDIEESIKSETSGDLEKAYLTLVSCAKDCPGYFAKLLHESMKGAGTDEDTLIRILVTRAESDLPAIKGKFQEMYKKSLTEAVRSDTSGDFRKLLLAILH

SEQ ID NO: 49 - protein, 317 aa, Meleagris gallopavo
MGSSHSTDKRRHHGFDADRDAKKLHSACKGAGTDEKKIIEVLSSRTSEQRQQIKQKYKALYGKDLEEVLKGDLSGSFEKAVLALLDLPCEYKARELHKAMKGAGTDESLLIEILCTQNNKEITSIKEAYKRLFDKDLESDVKGDTSGSLRKILVAVLEATRDENQQVNIELAEQDASDLYKAGEGRWGTEELAFNVVLAKRSYSQLRATFQAYEKVCGKDIEESIKSETSGDLEKAYLTLVSCAKDCPGYFAKLLHESMKGAGTDEDTLIRILVTRAESDLPAIKGKFQEMYKKSLTEAVRSDTSGDFRKLLLAILH

SEQ ID NO: 50 - protein, 316 aa, Oryctolagus cuniculus
MGNRHAKARSHQGFDVDRDVKKLNKACKGMGTDEAAIIEILSTRTSEQRQQIKQKYKTAYGKDLEEVLKGELSGNFEKAALALLDRPSEYAARLLQKAMKGLGTDEALLIEILCTTTNKEIIAIKEAYQRLFDRSLESDVKGDTSGNLERILVSLLQANRDEGDTVDKDLAGQDAKELYDAGEGRWGTDELAFNEVLARRNYKQLRATFQAYQMLIGKDIEAAIEEETSGDLKKAYLTLVKCAQDREGYFAERLYKSMKGAGTDEETLIGIIVTRAEVDLPGIKAKFQEKYQKSLSDMVRSDTSGDFRKLLVALLQ

SEQ ID NO: 51 - protein, 357 aa, Oryctolagus cuniculus
MGNRHSQSYSLSEGSQQLPKGDTQPSAATQPLSHLSGNGKPEAPQPAKARSHQGFDVDRDVKKLNKACKGMGTDEAAIIEILSTRTSEQRQQIKQKYKTAYGKDLEEVLKGELSGNFEKAALALLDRPSEYAARLLQKAMKGLGTDEALLIEILCTTTNKEIIAIKEAYQRLFDRSLESDVKGDTSGNLERILVSLLQANRDEGDTVDKDLAGQDAKELYDAGEGRWGTDELAFNEVLARRNYKQLRATFQAYQMLIGKDIEAAIEEETSGDLKKAYLTLVKCAQDREGYFAERLYKSMKGAGTDEETLIGIIVTRAEVDLPGIKAKFQEKYQKSLSDMVRSDTSGDFRKLLVALLQ

SEQ ID NO: 52 - protein, 383 aa, Ovis aries
MKYRTTVSEETKMGNRHSQSYSLPEGSQHLPKGQALPSADLQPLSHPSGSGEPESHQPAKGKSHQGLDAGRDTKAKSHQRFDADRDAKKLNKACKGMGTDEAAIIEILSSRTSSERQQIKQKYKTTYGKDLEEVLKSELSGNFEKAALALLDRPDEYAARQLQKAMKGLGTSEAVLIEVLCTRTNKEIIAIKEAYQRLFDKSLESDVKGDISGSLKRILVSLLQANRDEGDNVDKDLAGQDAKELYDAGEGRWGTDELAFNELLAKRSHKQLRATFEAYQTLIGKDIEEAIEAETSGDLQKAYLTLVRSARDLQGYFADRLYKSMKGAGTDEDTLIDIIVTRAEVDLQGIKARFQEKYQKSLSDMVRSDTSGDFQKLLVALLH

SEQ ID NO: 53 - protein, 342 aa, Ovis aries
MKYRTTVSEETKMGNRHAKGKSHQGLDAGRDTKAKSHQRFDADRDAKKLNKACKGMGTDEAAIIEILSSRTSSERQQIKQKYKTTYGKDLEEVLKSELSGNFEKAALALLDRPDEYAARQLQKAMKGLGTSEAVLIEVLCTRTNKEIIAIKEAYQRLFDKSLESDVKGDISGSLKRILVSLLQANRDEGDNVDKDLAGQDAKELYDAGEGRWGTDELAFNELLAKRSHKQLRATFEAYQTLIGKDIEEAIEAETSGDLQKAYLTLVRSARDLQGYFADRLYKSMKGAGTDEDTLIDIIVTRAEVDLQGIKARFQEKYQKSLSDMVRSDTSGDFQKLLVALLH

SEQ ID NO: 54 - protein, 345 aa, Salmo salar
MSATTSGKYEDISTIYLSDLNTAGQISVKMGNCQPTIMPCEDFDVAADIKALRKACKGLGTDEQVIIDVLTNRCAAQRMEIKQAYFDKYGNELVDVLKSELGGNFENAVVAMLDPPVVYAVKELRRAMKGAGTDEDTLVEILCTATNADIHMLKECYFQVHERDLESDIEGDTSGDVRNLLTALLQGTRDESYDVDEGLAEADATALFEAGEGCFGTDESTFNFILAKRNYLQLQATFKVYEQLSGTEILDAIDNEVSGTLKGCYITLVRVAKNPQLYFARRLNEAMKGAGTDEDTIIRIVVCRSEYDLETIKDMYLEKYDMSLKDAIKSECGGDFKRLLLAVCH

SEQ ID NO: 55 - protein, 357 aa, Sus scrofa
MGNRHSQSYSLSEGSHQVPNGHAQPSAAMQPLSHRSGSGQPESHQPAKAKSDQGFDVDRDAKKLHKACKGMGTDEAAIIEILSSRTSNERQQIKQKYKATYGKDLEEVLKSELSGNFEKTALALLDRPCEYAARQLRKAMKGLGTDESVLIEILCTRTNKEIIAIKEAYQKLFDRSLESDVKSDTSGNLKKILVSLLQANREEGDNVDKDLAGQDAKDLYDAGEGRWGTDELAFNEVLAQRSHKQLRATFQAYQVLIGKDIEEAIESETSGNLKKAYLTLVRSARDLQGYFADRLYKSMKGAGTDEDTLIDIIVTRAEVDLPAIKAKFQENYQTSLSDMVRADTSGDFRKLLVALLH

SEQ ID NO: 56 - protein, 323 aa, Sus scrofa
MGNRHSQSYSLSEGSHQVPNGHAQPSAAMQPLSHRSGSGQPESHQPAKAKSDQGFDVDRDAKKLHKACKGMGTDEAAIIEILSSRTSNERQQIKQKYKATYGKDLEEVLKSELSGNFEKTALALLDRPCEYAARQLRKAMKGLGTDESVLIEILCTRTNKEIIAIKEAYQKLFDRSLESDVKSDTSGNLKKILVSLLQANREEGDNVDKDLAGQDAKDLYDLIGKDIEEAIESETSGNLKKAYLTLVRSARDLQGYFADRLYKSMKGAGTDEDTLIDIIVTRAEVDLPAIKAKFQENYQTSLSDMVRADTSGDFRKLLVALLH

SEQ ID NO: 57 - protein, 316 aa, Sus scrofa
MGNRHAKAKSDQGFDVDRDAKKLHKACKGMGTDEAAIIEILSSRTSNERQQIKQKYKATYGKDLEEVLKSELSGNFEKTALALLDRPCEYAARQLRKAMKGLGTDESVLIEILCTRTNKEIIAIKEAYQKLFDRSLESDVKSDTSGNLKKILVSLLQANREEGDNVDKDLAGQDAKDLYDAGEGRWGTDELAFNEVLAQRSHKQLRATFQAYQVLIGKDIEEAIESETSGNLKKAYLTLVRSARDLQGYFADRLYKSMKGAGTDEDTLIDIIVTRAEVDLPAIKAKFQENYQTSLSDMVRADTSGDFRKLLVALLH

SEQ ID NO: 58 - protein, 316 aa, Thunnus albacares
MGNCQPTIVPYEDFDVVADIKAIRKACKGLGTDEQAIIDILANRSSFQRQEIKQAYFDKYDDELVDKLKSELSGNFEKAILAMLDPPVIYAVKELRKAMKGPGTDEDVLVEIICTATNADIALFKECYFQVHERDLEADIEGDTSGDVRNLLTSLLQGNRDESYEVDEGLAEEDATSLFEAGEGRFGTDESTFSHILSSRNYLQLQATFKIYEQLSGTEILDAIENETSGTLKKCYIALVRVAKNPQLYFARRLNKAMKGAGTDEDTLIRIIVCRAEYDLETIKDMYLEKYDVSLKDALRDECSGDFKRLLLAICH

SEQ ID NO: 59 - protein, 315 aa, Thunnus albacares
MGNCQPTIVPYEDFDVVADIKAIRKACKGLGTDEQAIIDILANRSSFQRQEIKQAYFDKYDDLVDKLKSELSGNFEKAILAMLDPPVIYAVKELRKAMKGPGTDEDVLVEIICTATNADIALFKECYFQVHERDLEADIEGDTSGDVRNLLTSLLQGNRDESYEVDEGLAEEDATSLFEAGEGRFGTDESTFSHILSSRNYLQLQATFKIYEQLSGTEILDAIENETSGTLKKCYIALVRVAKNPQLYFARRLNKAMKGAGTDEDTLIRIIVCRAEYDLETIKDMYLEKYDVSLKDALRDECSGDFKRLLLAICH

SEQ ID NO: 60 - protein, 358 aa, Anas platyrhynchos
MSTVHEILSKLSLEGDHSLPPSAYATVKAYSNFDADRDAAALEAAIKTKGVDEVTIINILTNRSNEQRQDIAFAYQRRTKKVCQTPNGSPTSQNAHPKPHELSAALKSALSGHLEAVILGLLKTPSQYDASELKAAMKGLGTDEDTLIEIICSRTNQELSEINRVYREMYKTELEKDIISDTSGDFRKLMVALAKGKRCEDTSVIDYELIDQDARELYDAGVKRKGTDVPKWINIMTERSVPHLQKVFERYKSYSPYDMLESIKKEVKGDLENAFLNLVQCIQNKQLYFADRLYDSMKGKGTRDKVLIRIMVSRCEVDMLKIKSEFKRKYGKSLYYFIQQDTKGDYQRALLNLCGGED

SEQ ID NO: 61 - protein, 339 aa, Anas platyrhynchos
MSTVHEILSKLSLEGDHSLPPSAYATVKAYSNFDADRDAAALEAAIKTKGVDEVTIINILTNRSNEQRQDIAFAYQRRTKKELSAALKSALSGHLEAVILGLLKTPSQYDASELKAAMKGLGTDEDTLIEIICSRTNQELSEINRVYREMYKTELEKDIISDTSGDFRKLMVALAKGKRCEDTSVIDYELIDQDARELYDAGVKRKGTDVPKWINIMTERSVPHLQKVFERYKSYSPYDMLESIKKEVKGDLENAFLNLVQCIQNKQLYFADRLYDSMKGKGTRDKVLIRIMVSRCEVDMLKIKSEFKRKYGKSLYYFIQQDTKGDYQRALLNLCGGED

SEQ ID NO: 62 - protein, 275 aa, Anas platyrhynchos
MNRGRILLLPIRGEQKKELSAALKSALSGHLEAVILGLLKTPSQYDASELKAAMKGLGTDEDTLIEIICSRTNQELSEINRVYREMYKTELEKDIISDTSGDFRKLMVALAKGKRCEDTSVIDYELIDQDARELYDAGVKRKGTDVPKWINIMTERSVPHLQKVFERYKSYSPYDMLESIKKEVKGDLENAFLNLVQCIQNKQLYFADRLYDSMKGKGTRDKVLIRIMVSRCEVDMLKIKSEFKRKYGKSLYYFIQQDTKGDYQRALLNLCGGED

SEQ ID NO: 63 - protein, 339 aa, Bos taurus
MSTVHEILCKLSLEGDHSTPPSAYGSVKAYTNFDAERDALNIETAIKTKGVDEVTIVNILTNRSNEQRQDIAFAYQRRTKKELASALKSALSGHLETVILGLLKTPAQYDASELKASMKGLGTDEDSLIEIICSRTNQELQEINRVYKEMYKTDLEKDIVSDTSGDFRKLMVALAKGRRAEDGSVIDYELIDQDARDLYDAGVKRKGTDVPKWISIMTERSVCHLQKVFERYKSYSPYDMLESIKKEVKGDLENAFLNLVQCIQNKPLYFADRLYDSMKGKGTRDKVLIRIMVSRSEVDMLKIRSEFKKKYGKSLYYYIQQDTKGDYQKALLYLCGGDD

SEQ ID NO: 64 - protein, 339 aa, Cervus elaphus; identical to SEQ ID NO: 63

SEQ ID NO: 65 - protein, 339 aa, Gallus gallus
MSTVHEILSKLSLEGDHSLPPSAYATVKAYSNFDADRDAAALEAAIKTKGVDEVTIINILTNRSNEQRQDIAFAYQRRTKKELSAALKSALSGHLEAVILGLLKTPSQYDASELKAAMKGLGTDEDTLIEIICSRTNQELNEINRVYREMYKTELEKDIISDTSGDFRKLMVALAKGKRCEDTSVIDYELIDQDARELYDAGVKRKGTDVPKWINIMTERSVPHLQKVFERYKSYSPYDMLESIKKEVKGDLENAFLNLVQCIQNKQLYFADRLYDSMKGKGTRDKVLIRIMVSRCEVDMLKIKSEFKRKYGKSLYYFIQQDTKGDYQRALLNLCGGED

SEQ ID NO: 66 - protein, 406 aa, Meleagris gallopavo
MKLNLARDVKNNKKVFYRYIDQKRWTKECVPPLLNENKELASTDTEKAEVLSLNMSQQGVLAAWKANGVLGSIRRGVGSRDRGHSLPPSAYATVKAYSNFDADRDAAALEAAIKTKGVDEVTIINILTNRSNEQRQDIAFAYQRRTKKELSAALKSALSGHLEAVILGLLKTPSQYDASELKAAMKGLGTDEDTLIEIICSRTNQELNEINRVYREMYKTELEKDIISDTSGDFRKLMVALAKGKRCEDTSVIDYELIDQDARELYDAGVKRKGTDVPKWINIMTERSVPHLQKVFERYKSYSPYDMLESIKKEVKGDLENAFINLVQCIQNKQLYFADRLYDSMKGKGTRDKVLIRIMVSRCEVDMLKIKSEFKRKYGKSLYYFIQQDTKGDYQRALLNLCGGED

SEQ ID NO: 67 - protein, 339 aa, Oryctolagus cuniculus
MSTVHEILCKLSLEGDHSTPPSAYGSVKAYGNFDAERDALNIETAIKTKGVDEVTIVNILTNRSNAQRQDIAFAYQRRTKKELPAALKSALSGHLETVILGLLKTPAQYDASELKASMKGLGTDEDSLIEIICSRTNQELQEINRVYKEMYKTDLEKDIVSDTSGDFRKLMVALAKGRRAEDGSVIDYELIDQDARDLYDAGVKRKGTDVPKWISIMTERSVCHLQKVFDRYKSYSPYDMLESIKKEVKGDLENAFLNLVQCIQNKPLYFADRLYDSMKGKGTRDKVLIRIMVSRSEVDMLKIRSEFKRKYGKSLYSYIQQDTKGDYQKALLYLCGGDD

SEQ ID NO: 68 - protein, 339 aa, Ovis aries; identical to SEQ ID NO: 63

SEQ ID NO: 69 - protein, 372 aa, Salmo salar
MQNLPVVFKHHTHSKPHSQDYFTAPQLSSAQSGLNMALVSEFLGQLSLNMGSCEPKYPTVVPAIDFDPDKDAARIETAIKTKGVDEATIIAVLTKRTYSQRREVAFAYERRAKKDLVTALKAALSGPLETVILGLMKSTAQYDASEIKGSIKGLGTDEEVLIEIVCSRSSSELSEIKTVYKELFKKDLEKDVAGDTSGDFRSLLLALVQAKRAEPSSVVDYEKIDEDARALYDAGVKRKGTDVATWISIMSERSVPHLQKVFDRYKSYSPYDMQESIRKEVKGDLEKSFLTLVECFENKPLYFASRLAEAMKGKSAKEKVVTRIMVSRCEVDLMKIRTEFKSQTGKSLFQTISEHTKGDYQKAMLSLCGGDD

SEQ ID NO: 70 - protein, 315 aa, Salmo salar
MDIEYLPSFEMAWGTLGTIRPFLNFHAKRDALNIQAALEQKDVLTLINILTNRSNAQRQIIAQTFKTITEKELSSGLKKALSGELENLLLALLMTPLQFEAYRLRQSMEGIGTDEEGLLEILCTRSPKVLANITATYKEQYNKDLETDLKGETSGDFAKLVLALLKKKKVVGLVQRDTEALSEAINQAKADPGPWITILTTRDSEHLNKVFVNLESEKQETVDEAIEKAFSGDVRLGLKTLVRCVQSPNLYLAQRLETMKAAIVQGVMVSHSEKDLLCVRVAYLRQTGTSLYTALQKQFKGDHLQALLAICRSED

SEQ ID NO: 71 - protein, 320 aa, Salmo salar
MDIEYLPSFEMAWGTLGTIRPFLNFHAKRDALNIQAALEQKDVLTLINILTNRSNAQRQIIAQTFKTITEKELSSGLKKALSGELENLLLALLMTPLQFEAYRLRQSMEGIGTDEEGLLEILCTRSPKVLANITATYKEQYNKDLETDLKGETSGDFAKLVLALLKKKKVVGLVQRDTEALSEAINQAKADPGPWITILTTRDSEHLNKVFVNLESEKQETVDEAIEKAFSGDVRLGLKTLGKTLELRCVQSPNLYLAQRLETMKAAIVQGVMVSHSEKDLLCVRVAYLRQTGTSLYTALQKQFKGDHLQALLAICRSED

SEQ ID NO: 72 - protein, 341 aa, Salmo salar
MDIEYLPSFEMAWGTLGTIRPFLNFHAKRDALNIQAALEQKDVLTLINILTNRSNAQRQIIAQTFKTITEKELSSGLKKALSGELENLLLALLMTPLQFEAYRLRQSMEGIGTDEEGLLEILCTRSPKVLANITATYKEQYNKDLETDLKGETSGDFAKLVLALLKKKKVVGLVQRDTEALSEAINQAKADPGPWITILTTRDSEHLNKVFVNLESEKQETVDEAIEKAFSGDVRLGLKTLGKTLELRCVQSPNLYLAQRLETMKAAIVQGVMVSHSEKDLLCVRVAYLRQTGTSLYTALQGAFPSPGWPFGKFWAELEYTHFSRHHYTTVCTHPPDVMHI

SEQ ID NO: 73 - protein, 312 aa, Salmo salar
MDIEYLPSFEMAWGTLGTIRPFLNFHAKRDALNIQAALEQKDVLTLINILTNRSNAQRQIIAQTFKTITEKELSSGLKKALSGELENLLLALLMTPLQFEAYRLRQSMEGIGTDEEGLLEILCTRSPKVLANITATYKEQYNKDLETDLKGETSGDFAKLVLALLKKKKVVGLVQRDTEALSEAINQAKADPGPWITILTTRDSEHLNKVFVNLESEKQETVDEAIEKAFSGDVRLGLKTLGKTLELRCVQSPNLYLAQRLETMKAAIVQGVMVSHSEKDLLCVRVAYLRQTGTSLYTALQSLQWTMCCSPG

SEQ ID NO: 74 - protein, 336 aa, Salmo salar
MDIEYLPSFEMAWGTLGTIRPFLNFHAKRDALNIQAALEQKDVLTLINILTNRSNAQRQIIAQTFKTITEKELSSGLKKALSGELENLLLALLMTPLQFEAYRLRQSMEGIGTDEEGLLEILCTRSPKVLANITATYKEQYNKDLETDLKGETSGDFAKLVLALLKKKKVVGLVQRDTEALSEAINQAKADPGPWITILTTRDSEHLNKVFVNLESEKQETVDEAIEKAFSGDVRLGLKTLVRCVQSPNLYLAQRLETMKAAIVQGVMVSHSEKDLLCVRVAYLRQTGTSLYTALQGAFPSPGWPFGKFWAELEYTHFSRHHYTTVCTHPPDVMHI

SEQ ID NO: 75 - protein, 307 aa, Salmo salar
MDIEYLPSFEMAWGTLGTIRPFLNFHAKRDALNIQAALEQKDVLTLINILTNRSNAQRQIIAQTFKTITEKELSSGLKKALSGELENLLLALLMTPLQFEAYRLRQSMEGIGTDEEGLLEILCTRSPKVLANITATYKEQYNKDLETDLKGETSGDFAKLVLALLKKKKVVGLVQRDTEALSEAINQAKADPGPWITILTTRDSEHLNKVFVNLESEKQETVDEAIEKAFSGDVRLGLKTLVRCVQSPNLYLAQRLETMKAAIVQGVMVSHSEKDLLCVRVAYLRQTGTSLYTALQSLQWTMCCSPG

SEQ ID NO: 76 - protein, 339 aa, Sus scrofa
  VARIANT 290: note = Any amino acid
MSTVHEILCKLSLEGDHSTPASAYGSVKAYTNFDAERDALNIETAIKTKGVDEVTIVNILTNRSNEQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVCHLQKVFERYKSYSPYDMLESIKKEVKGDLENAFLNLVQCIQNKPLYFADRLYDSMKGKGTRDKVLIXIMVSRSEVDMLKIRSEFKRKYGKSLYNYIQQDTKGDYQKALLYLCGGDD

SEQ ID NO: 77 - protein, 315 aa, Thunnus albacares
MDPEYFKSYEMCWGTLGTVRPFSNFHQDRDAKEIQAALEKKDAGTLVRILTNRSNAQRQVIAKTFEDITQKDLAAGLKKALAGDLETLLLELLMPPLEYEAHRLQQAMAGLGTDEETLMEILCTRSGKQLQEISAAYTYLYKKDLEKELKGETSGDFAKLIVALLHKENVTGGVQKDIQSLSASLNGKKADAGPWIDILTSRESDHLNKVLTGLELESGLMLDQALEKRFTGDIRLGLRVLVQCIQSPDAYLAKRLTTKKASTVQGIMVSHCEEDLLCIRAAYLKLTGTSLYTALQTQFKGDYLQALLAICRSED

SEQ ID NO: 78 - protein, 323 aa, Bos taurus
MASIWVGKRGTIRDYAGFNPSVDAEAIRKAIRGIGTDEKTLISILTERTNAQRLLIAKEYQALCGKELKDDLKGDLSGHFKHLMVALVTPPAVFDAKQLKKSMKGMGTNEDALIEILTTRTSKQMQEIGHAYYTAYKKSLGDEISSETSGDFRKALLILANGRRDESLKVDEQLARKDAQILYNAGEKRWGTDEDAFTNILCLRSFPQLKLTFDEYRNISQKDIEDSIKGELSGHFEDLLLAIVRCARNTPAFLAERLYRALKGAGTDEFTLNRIMVSRSEIDLLDIRAEFKKLSGYSLYSAIKSDTSGDYEITLLKICGGDD

SEQ ID NO: 79 - protein, 323 aa, Bubalus bubalis
MASIWVGKRGTIRDYSGFNPSVDAEAIRKAIRGIGTDEKTVISILTERTNAQRQLIAKEYQALCGKELKDDLKGDLSGHFKHLMVGLVTPPAVFDAKQLKKSMKGMGTNEDALIEILTTRTSKQMQEVGQAYYTAYKKSLGDEISSETSGDFRKALLILANGRRDESLKVDEQLARKDAQILYNAGEKRWGTDEDAFTDVLCLRSFPQLKLTFDEYRNISQKDIEDSIKGELSGHFEDLLLAIVRCARNTPAFLAERLYRALKGAGTDEFTLNRIMVSRSEIDLLDIRAEFKKLSGYSLYSAIKSDTSGDYEITLLKICGGDD

SEQ ID NO: 80 - protein, 323 aa, Cervus elaphus
MASIWVGKRGTIRDYPGFNPSVDAEAIRKAIRGIGTDEKTLISVLTERTNAQRQLIAKEYQAICGKELKDDLKGDLSGHFKDLMVALVTPPAVFDAKQLKKSMKGTGTNEDALIEILTTRTSKQMQEIGHAYYTVYKKSLGDEISSETSGDFRKALLILANGRRDDSLKVDEQLARKDAQILYNAGEKRWGTDEDAFTDILCLRSFPQLKLTFDEYRNISQKDIEDSIKGELSGHFEDLLLAIVRCARNTPAFLAERLYQALKGAGTDEFTVNRIMVSRSEIDLLDIRAEFKKLSGYSLYSAIKSDTSGHYESTLLKICGGDD

SEQ ID NO: 81 - protein, 323 aa, Oryctolagus cuniculus
MASLWVGHRGTVRDYPGFSPSVDAEAIRKAIRGIGTDEKTLISILAERSSAQRQLIVKEYQAAYGKELKDDLKGDLSGHFEHLMVALVTPPAVFDAKQLKKSMKGTGTDEDALIEILTTRTSRQMKEISQAYYTVYKKSLGDDISSETSGDFRKALLTLADGRRDESVKVDEHLAKKDAQILYNAGENKWGTDEDKFTEILCLRSFLQLKLTFDEYRNISQKDIEDSIKGELSGHFEDLLLAIVHCVRNLPAFLAARLYQALKGAGTDEFTLNRIMVSRSEIDLLDIRAEFKKQYGYSLYSAIKSDTSGDYEVTLLKICGGDD

SEQ ID NO: 82 - protein, 323 aa, Ovis aries
MASIWVGKRGTIRDHSGFNPSVDAEAIRKAIRGIGTDEKTLISILTERTNAQRQLIAKEYQAVCGKELKDDLKGDLSGHFKGLMVALVTPPAVFDAKQLKKSMKGTGTNEDALIEILTTRTSKQMQEIGHAYYTVYKKSLGDEISSETSGDFRKALLILANGRRDESLKVDEQLAKKDAQILYNAGEKRWGTDEDAFTDILCLRSFPQLKLTFDEYRNISQKDIEDSIKGELSGHFEDLLLAIVRCARNTPAFLAERLYQALKGAGTDEFTLNRIMVSRSEIDLLDIRAEFKKLSGYSLYSAIKSDTSGDYEITLLKICGGDD

SEQ ID NO: 83 - protein, 339 aa, Salmo salar
MANVWGDLDLLLDSPSSLTLTSTARGTIKEKAGFKAAEDVVALRKAIEGIGTTEKTLVEVLTGRSNAQRQLICKAYQEAANRTLVDDLKGDTHGDFEDLLVALVTPPALFDCHEVIRAIKGPGTTEATLTEIFASRSNKQIKALSDAYLAETGRSMTHDLKSEVSGDYATTLLILAEGKRDESTNVDTAKAKADAKALYEAGEKKWGTDESKFIDILCHRSVPQLRQTLVEYKALSNKTLQESIEREMSGNLEDLLVAIVKCVKSVPAYLAERLFKSTKGVGTTESILTRIMVGRSEMDMLDIRAEFKKLFGCSLYSTIKSETAGSYGQALLKLCGEDD

SEQ ID NO: 84 - protein, 120 aa, Sus scrofa
MASVWVGHRGTMRDHPGFNPSVDAEAIHKAVREIGTDEKTHISILTERSNVQRQLTVKEYQAAYGKVAEPVDPKGAVALAGTRWTSGRHGKLLVEHRQELGREDLHPIRNTHQLSDPRGL

SEQ ID NO: 85 - protein, 315 aa, Anas platyrhynchos
MATIKGVSSFSAEQEAQALRKAMKGFGTDEDAIIDILTKLNVSQRQQVLITYKSTIGRDLIDDLKSELSGNFERVIIGMMTPTTMYDVHELKRALKGAGTDEGCLIEILASRTNEEIRRINETYRLQYGSSLEDDIVSDTSSMFRRVLVSLATGNRDEGMYVDDALAQQDAQCLYEAGEKKWGTDEVQFMSILCTRNRCHLLRVFDVYRGIANKDITDSIKSEMSGDLEDALLAVVKCMRNKPAYFAERLYKSMKGLGTDDNTLIRVMVSRSEIDMLDIRREFLTMYGKSLYSFIKGDCSGDYRKVLLRLCGGED

SEQ ID NO: 86 - protein, 319 aa, Bos taurus
MAAKGGTVKAASGFNAAEDAQTLRKAMKGLGTDEDAIINVLAYRSTAQRQEIRTAYKTTIGRDLMDDLKSELSGNFEQVILGMMTPTVLYDVQELRKAMKGAGTDEGCLIEILASRTPEEIRRINQTYQLQYGRSLEDDIRSDTSFMFQRVLVSLSAGGRDESNYLDDALMRQDAQDLYEAGEKKWGTDEVKFLTVLCSRNRNHLLHVFDEYKRIAQKDIEQSIKSETSGSFEDALLAIVKCMRNKSAYFAERLYKSMKGLGTDDDTLIRVMVSRAEIDMLDIRANFKRLYGKSLYSFIKGDTSGDYRKVLLILCGGDD

SEQ ID NO: 87 - protein, 319 aa, Cervus elaphus
MAAKGGTVKAASGFNAAEDAQTLRKAMKGLGTDEDAIINVLAYRSTAQRQEIRTAYKTTIGRDLLDDLKSELSGNFEQVILGMMTPTVLYDVQELRKAMKGAGTDEGCLIEILASRTPEEIRRINQTYQLQYGRSLEDDIRSDTSFMFQRVLVSLSAGGRDESNYLDDALVRQDAQDLYEAGEKKWGTDEVKFLTVLCSRNRNHLLHVFDEYKRIAQKDIEQSIKSETSGSFEDALLAIVKCMRNKSAYFAERLYKSMKGLGTDDDTLIRVMVSRAEIDMLDIRANFKRLYGKSLYSFIKGDTSGDYRKVLLILCGGDD

SEQ ID NO: 88 - protein, 315 aa, Gallus gallus
MATIKGVSSFNPEQEAQALRKAMKGFGTDEDAIIDILTKLNVSQRQQVLITYKSTIGRDLIDDLKSELSGNFERVIIGMMTPTTMYDVHELRRAVKGAGTDEGCLIEILASRTNEEIRRINENYKLQYGCSLEDDIVSDTSSMFRRVLVSLATGNRDEGTYVDDALAQQDAQCLYEAGEKKWGTDEVQFMSILCTRNRYHLLRVFDVYGGIANKDITDSIKSEMSGDLEDALLAVVKCMRNKPAYFAERLYKSMKGLGTDDNTLIRVMVSRSEIDMLDIRREFLTMYGKSLYSFIKGDCSGDYRKVLLKLCGGED

SEQ ID NO: 89 - protein, 359 aa, Meleagris gallopavo
MLAHGGSIFPDKLSGHTAPSWFNPQGSTHAVVIPVPVFQSPGSMKATIKGVSSFSPEQEAQALRKAMKGFGTDEDAIIDVLTKLNVSQRQQVLITYKSTIGRDLMDDLKSELSGNFERVIIGMMTPTTMYDVHELRRAVKGAGTDEGCLIEILASRTNEEIRRINENYKLQYGCSLEDDIVSDTSSMFRRVLVSLATGNRDEGMYVDDGLAQQDAQCLYEAGEKKWGTDEVQFMSILCTRNRYHLLRVFDVYRGIANKDITDSIKSEMSGDLEDALLAVVKCVRNKPAYFAERLYKSMKGLGTDDSTLIRVMVSRSEIDMLDIRREFLTMYGKSLYSFIKGDCSGDYRKVLLKLCGGED

SEQ ID NO: 90 - protein, 315 aa, Meleagris gallopavo
MATIKGVSSFSPEQEAQALRKAMKGFGTDEDAIIDVLTKLNVSQRQQVLITYKSTIGRDLMDDLKSELSGNFERVIIGMMTPTTMYDVHELRRAVKGAGTDEGCLIEILASRTNEEIRRINENYKLQYGCSLEDDIVSDTSSMFRRVLVSLATGNRDEGMYVDDGLAQQDAQCLYEAGEKKWGTDEVQFMSILCTRNRYHLLRVFDVYRGIANKDITDSIKSEMSGDLEDALLAVVKCVRNKPAYFAERLYKSMKGLGTDDSTLIRVMVSRSEIDMLDIRREFLTMYGKSLYSFIKGDCSGDYRKVLLKLCGGED

SEQ ID NO: 91 - protein, 321 aa, Oryctolagus cuniculus
MAMASKGGTIKAASGFNAAEDAQALRKAMKGLGTDEDAIIQVLAYRNTAQRQEIRTAYKSTIGRDLVDDLKSELSGNFEQVIVGMMMPTVLYDVQELRKALKGAGTDEGCLIEILASRTPEEIRRINQTYQQQYGRSLEDDICSDTSFMFQRVLVSLSAGGRDEGNYLDDDLVRQDAQDLYEAGEKKWGTDEVKFLTVLCSRNRNHLLHVFDEYKRISQKDIEQSIKSETSGSFEDALLAIVKCMRNKPAYFAERLYKSMKGLGTDDDTLIRVMVSRAEIDMMDIRANFRKLYGKSLYSFIKGDTSGDYRKVLLVLCGGDD

SEQ ID NO: 92 - protein, 319 aa, Oryctolagus cuniculus
MASKGGTIKAASGFNAAEDAQALRKAMKGLGTDEDAIIQVLAYRNTAQRQEIRTAYKSTIGRDLVDDLKSELSGNFEQVIVGMMMPTVLYDVQELRKALKGAGTDEGCLIEILASRTPEEIRRINQTYQQQYGRSLEDDICSDTSFMFQRVLVSLSAGGRDEGNYLDDDLVRQDAQDLYEAGEKKWGTDEVKFLTVLCSRNRNHLLHVFDEYKRISQKDIEQSIKSETSGSFEDALLAIVKCMRNKPAYFAERLYKSMKGLGTDDDTLIRVMVSRAEIDMMDIRANFRKLYGKSLYSFIKGDTSGDYRKVLLVLCGGDD

SEQ ID NO: 93 - protein, 319 aa, Salmo salar
MAAIGNRGTVVEAAAFNVEEDVNRLRGAMKGAGTDEAAVIEVLARRTIAQRQSIKEAYKLTVGKDLADDLQGELTGNIEKVVLGLLMIAPKYDAYELRTAIKGSGTEEAALIDILASRTNAEIRAITEVYMKEHGKSLEDDIEADTSGMFKRVLVSLLTAGRDESNSVNETQAVQDAKEIYEAGEACWGTDEVKFLTVLCVRNRNHLLRVFEEYQKISGRDIEDSIKREMSGSLEDVFLAIVKCLRNKPAFFAERLYKSMKGLGTTDSVLIRIMVARAEIDMLDIKTEFSKAYGKTLHSFIHGDSSGDYRKILLELCGE

SEQ ID NO: 94 - protein, 319 aa, Sus scrofa
MAAKGGTVKAASGFNAAEDAQTLRKAMKGLGTDEDAIISVLAYRSTAQRQEIRTAYKSTIGRDLLDDLKSELSGNFEQVILGMMTPTVLYDVQELRRAMKGAGTDEGCLIEILASRTPEEIRRINQTYQLQYGRSLEDDIRSDTSFMFQRVLVSLSAGGRDEGNYLDDALVRQDAQDLYEAGEKKWGTDEVKFLTVLCSRNRNHLLHVFDEYKRISQKDIEQSIKSETSGSFEDALLAIVKCMRNKSAYFAERLYKSMKGLGTDDNTLIRVMVSRAEIDMMDIRANFKRLYGKSLYSFIKGDTSGDYRKVLLILCGGDD

SEQ ID NO: 95 - protein, 320 aa, Thunnus albacares
MAAIGNRGAVTEAAGFDAEADVQKLRGAMKGAGTDEAAVIDVLSHRTIAQRQRIKEAYKQAVGKDLADDLSSELSGNFRSVVLGLLMLAPVYDAHELRSAIKGAGTEEACLIDILASRSNAEINTINAFYKKEHGKSLEDDVCGDTSGMFQRVLVSLLTAGRDESDNVDEAQAMQDAKEIYEAGEARWGTDEVKFLTVLCVRNRKHLLRVFDEYQKISGRDIEDSIKREMSGCLEDVFLAIVKCLRNKPAFFAERLYKSMKGLGTTDSVLIRIMVSRAEIDMLDIKAQFLKMYGKTLYSFIKGDTSGDYRKILLELCGGE

SEQ ID NO: 96 - protein, 321 aa, Anas platyrhynchos
MAKTTRGTVTAFSPFDARADAEALRKAMKGMGTDEDTILKILTSRNNAQRQEIASAFKTLFGRDLVDDLKSELTGKFETLMVSLMRPTCIFDAHALKHAIKGAGTNEKVLTEILASRTPAEVRNIKQVYLQEYEANLEDKITGETSGHFQRMLVVLLQANRDPEGRVDEGLVEQDAQVLFRAGELKWGTDEEKFITILGTRSVSHLRRVFDKYMTISGFQIEETIDRETSGDLEKLLLAVVKCIRSVPAYFAETLYYSMKGAGTDDDTLIRVMVSRSEIDLLDIRQEFRKNFAKSLHQMIQKDTSGDYRKALLLLCGGDDE

SEQ ID NO: 97 - protein, 289 aa, Anas platyrhynchos
MAKTTRGTVTAFSPFDARADAEALRKAMKGMGTDEDTILKILTSRNNAQRQEIASAFKTLFGRDLVDDLKSELTGKFETLMVSLMRPTCIFDAHALKHAIKGAGTNEKVLTEILASRTPAEVRNIKQVYLQEYEANLEDKITGETSGHFQRMLVVLLQANRDPEGRVDEGLVEQDAQVLFRAGELKWGTDEEKFITILGTRSVSHLRRVFDKYMTISGFQIEETIDRETSGDLEKLLLAVVKCIRSVPAYFAETLYYSMKDTVSLSICKRFGVCRSGTGYKQFMCLSLG

SEQ ID NO: 98 - protein, 321 aa, Bos taurus
MAQVLRGTVADFPGFDERADAETLRKAMKGLGTDEESILTLLTSRSNAQRQEIAVAFKTLFGRDLLDDLKSELTGKFEKLIVALMKPSRLYDAYELKHALKGAGTDEKVLTEIIASRTPEELRAIKQVYEEEYGSSLEDDVVGDTSGYYQRMLVVLLQANRDPDARIDEAQVEQDAQALFQAGELKWGTDEEKFITIFGTRSVSHLRRVFDKYMTISGFQIEETIDRETSGNLEQLLLAVVKSIRSIPAYLAETLYYAMKGAGTDDHTLIRVVVSRSEIDLYNIRKEFRKNFGTSLYSMIKGDTSGDYKKALLLLCGGEDD

SEQ ID NO: 99 - protein, 321 aa, Bubalus bubalis
MAQVLRGTVTDFPGFDERADAETLRKAMKGLGTDEESILTLLTSRSNAQRQEIAVAFKTLFGRDLLDDLKSELTGKFEKLIVALMKPSRLYDAYELKHALKGAGTDEKVLTEIIASRTPEELRAIKQVYEKEYGSSLEDDVVGDTSGYYQRMLVVLLQANRDPDTRIDEAQVEQDAQALFQAGELKWGTDEEKFITIFGTRSVSHLRRVFDKYMTISGFQIEETIDRETSGNLEQLLLAVVKSIRSIPAYLAETLYYAMKGAGTDDHTLIRVVVSRSEIDLYNIRKEFRKNFGTSLYSMIKGDTSGDYKKALLLLCGGEDD

SEQ ID NO: 100 - protein, 423 aa, Cervus elaphus
MFQSSGGRAPPRRRGLGAAAAHAGALRPEAVGGAQGRCGPRPAGLGWAGPDRAAVFPLPGAASKQRPQRPPLTLAGSARPAAFPAVRVWSCVAHLLLTWAGAMAQVLRGTVTDFPGFDERADAETLRKAMKGLGTDEESILTLLTSRSNAQRQEIAVAFKTLFGRDLLDDLKSELTGKFEKLIVALMKPSRLYDAYELKHALKGAGTNEKVLTEIIASRTPEELRAIKKVYEEEYGSSLEDDVVGDTSGYYQRMLVVLLQANRDPDTRIDEAQVEQDAQALFQAGELKWGTDEEKFITIFGTRSVSHLRRVFDKYMTISGFQIEETIDRETSGNLEQLLLAVVKSIRSIPAYLAETLYYAMKGAGTDDHTLIRVVVSRSEIDLYNIRKEFRKNFATSLYSMIKGDTSGDYKKALLLLCGGEDD

SEQ ID NO: 101 - protein, 353 aa, Cervus elaphus
MCEREPSRKPRVAGVRTKASEANAFLFSFDLILFSVLRGTVTDFPGFDERADAETLRKAMKGLGTDEESILTLLTSRSNAQRQEIAVAFKTLFGRDLLDDLKSELTGKFEKLIVALMKPSRLYDAYELKHALKGAGTNEKVLTEIIASRTPEELRAIKKVYEEEYGSSLEDDVVGDTSGYYQRMLVVLLQANRDPDTRIDEAQVEQDAQALFQAGELKWGTDEEKFITIFGTRSVSHLRRVFDKYMTISGFQIEETIDRETSGNLEQLLLAVVKSIRSIPAYLAETLYYAMKGAGTDDHTLIRVVVSRSEIDLYNIRKEFRKNFATSLYSMIKGDTSGDYKKALLLLCGGEDD

SEQ ID NO: 102 - protein, 324 aa, Gallus gallus
MEKLKDYTRGTVTAFSPFDARADAEALRKAMKGMGTDEETILKILTSRNNAQRQEIASAFKTLFGRDLVDDLKSELTGKFETLMVSLMRPARIFDAHALKHAIKGAGTNEKVLTEILASRTPAEVQNIKQVYMQEYEANLEDKITGETSGHFQRLLVVLLQANRDPDGRVDEALVEKDAQVLFRAGELKWGTDEETFITILGTRSVSHLRRVFDKYMTISGFQIEETIDRETSGDLEKLLLAVVKCIRSVPAYFAETLYYSMKGAGTDDDTLIRVMVSRSEIDLLDIRHEFRKNFAKSLYQMIQKDTSGDYRKALLLLCGGDDE

SEQ ID NO: 103 - protein, 321 aa, Gallus gallus
MAKYTRGTVTAFSPFDARADAEALRKAMKGMGTDEETILKILTSRNNAQRQEIASAFKTLFGRDLVDDLKSELTGKFETLMVSLMRPARIFDAHALKHAIKGAGTNEKVLTEILASRTPAEVQNIKQVYMQEYEANLEDKITGETSGHFQRLLVVLLQANRDPDGRVDEALVEKDAQVLFRAGELKWGTDEETFITILGTRSVSHLRRVFDKYMTISGFQIEETIDRETSGDLEKLLLAVVKCIRSVPAYFAETLYYSMKGAGTDDDTLIRVMVSRSEIDLLDIRHEFRKNFAKSLYQMIQKDTSGDYRKALLLLCGGDDE

SEQ ID NO: 104 - protein, 319 aa, Meleagris gallopavo
MYTRGTVTAFSPFDARADAEALRKAMKGMGTDEETILKILTSRNNAQRQEIASAFKTLFGRDLVDDLKSELTGKFETLMVSLMRPARIFDAHALRHAIKGAGTNEKVLTEILASRTPAEVQNIKQVYMQEYEANLEDKITGETSGHFQRLLVVLLQGNRDPDGRVDEALVEKDAQVLFRAGELKWGTDEETFITILGTRSVSHLRRVFDKYMTISGFQIEETIDRETSGDLEKLLLAVVKCIRSVPAYFAETLYYSMKGAGTDDDTLIRVMVSRSEIDLLDIRHEFRKNFAKSLHQMIQKDTSGDYRKALLLLCGGDDE

SEQ ID NO: 105 - protein, 321 aa, Oryctolagus cuniculus
MAQALRGTVTAFPGFDERADAEILRKAMKGLGTDEDSILTLLTARSNGQRQEIADAFKTLYGRDLLDDLKSELTGKFEKLIVALMKPSRLYDAYELKHALKGAGTDEKVLTEIIASRTPAELTAIKQVYEEEYGSSLEDDVMGDTSGYYQRMLVVLLQANRDPDSGIDTAQVEQDAQTLFQAGELKWGTDEEKFITIFGTRSVSHLRRVFDKYMTISGFQIEETIDRETSGNLEQLLLAVVKSIRSIPAYLAETLYYAMKGAGTDDHTLIRVMVSRSEIDLYNIRKEFRKNFGTSLYSMIKDDTSGDYKKALLLLCGGDDD

SEQ ID NO: 106 - protein, 328 aa, Ovis aries
MAQVLRGTVTDFPGFNERADAETLRKAMKGLGTDEESILTLLTSRSNAQRQEIAVAFKTLFGRDLLDDLKSELTGKFEKLIVALMKPSRLYDAYELKHALKGAGTDEKVLTEIIASRTPEELRAIKQVYEEEYGSSLEDDVVGDTSGYYQRMLVVLLQANRDPDARIDEAQVEQDAQALFQAGELKWGTDEEKFITIFGTRSVSHLRRVFDKYMTISGFQIEETIDRETSGNLEQLLLAVVKSIRSIPAYLAETLYYAMKGAGTDDHTLIRVVVSRSEIDLYNIRKEFRKNFGTSLYSMIKVVKPFLKLCLEILSKTINSVEDMHGRT

SEQ ID NO: 107 - protein, 321 aa, Ovis aries
MAQVLRGTVTDFPGFNERADAETLRKAMKGLGTDEESILTLLTSRSNAQRQEIAVAFKTLFGRDLLDDLKSELTGKFEKLIVALMKPSRLYDAYELKHALKGAGTDEKVLTEIIASRTPEELRAIKQVYEEEYGSSLEDDVVGDTSGYYQRMLVVLLQANRDPDARIDEAQVEQDAQALFQAGELKWGTDEEKFITIFGTRSVSHLRRVFDKYMTISGFQIEETIDRETSGNLEQLLLAVVKSIRSIPAYLAETLYYAMKGAGTDDHTLIRVVVSRSEIDLYNIRKEFRKNFGTSLYSMIKGDTSGDYKKTLLLLCGGEDD

SEQ ID NO: 108 - protein, 317 aa, Salmo salar
MAGRGSVRASGNFNANQDAETLYKAMKGLGTDEDSIMKLLTSRSNSQRQQIKAAYKTLHGKDLVGDLQGELGGKFETLVVALMTPPILYDATTLRNAIKGAGTDEKVLIEILSSRTAQQVKDIIAAYRQEFDADLEEDVTGDTSGHFRRLLVILLQASRQQGVQEGNIETDAQTLFSAGEKNYGTDEDQFITILGNRSAEHLRRVFAAYMKLAGYEMEESVKRETSGGLRDLLLAVVKCARSVPAYFAETLYYSMSGAGTDDQALIRVMVSRSEVDMLDIRADYRRLFAKSLYSTIQGDTSGDCRKALLLLCGGDDA

SEQ ID NO: 109 - protein, 321 aa, Sus scrofa
MAQVLRGTVTDFPGFDERADAETLRKAMKGLGTDEESILTLLTSRSNAQRQEIAVAFKTLFGRDLLDDLKSELTGKFEKLIVALMKPSRLYDAYELKHALKGAGTDEKILTEIIASRTPEELRAIKQVYEEEYGSSLEDDVVGDTSGYYQRMLVVLLQANRDPDGRIDEAQVEQDAQALFQAGELKWGTDEEKFITIFGTRSVSHLRRVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGGEDD

SEQ ID NO: 110 - protein, 672 aa, Anas platyrhynchos
MAPKGKVYRGSVKEFPGFDASQDAEALYNAMKGLGSDKEAILDLITSRSNKQRVEICQAYKSLYGKDLIADLKYELTGKFERLIVSLMRPPAYSDAKEIKDAIGGLGTDEKCLIEILASRTNQEIHDLVAAYKDAYERDLEADIVGDTSGHFKKMLVVLLQGAREEDDVVSEDLVEQDAKDLLEAGELKWGTDEAQFIYILGRRSKQHLRMVFNEYLKISGKPIERSIKGELSGDFEKLMLAVVKCIRSTAEYFAERLYKAMKGLGTRDNTLIRIMVSRSEIDMLDIREVFRTKYEKSLHNMIKEDTSGEYKKALLKLCGGDDDAAGEFFPEAAQVAYQMWEHSALAKVKLQGTVQPAASFNDDGDAQVLRKAMKGLGTDEGAIIDVVTKRSNAQRQQIIKAYKAHYGRDLMADLKSELSGSLAKLILGLMLTPAQYDAKQLRKAVEGAGTDESVLIEIMATRNNQEIAAINEAYQQAYHKRLEDDLSSDTSGHFKRILVSLALGNRDEGPENLTQAHEDAKVVAETLKLADVSSNDSSDSLETRFLSILCTRSYPQLRRVFQEFIKMTNHDVEHAIKKRMSGDVRDAFVAIVRSVKNKPAFFADKLYKSMKGAGTDERTLTRIMISRSEIDLLNIRAEFVDLFDKSLHHMIEKDTSGDYRMALLALCGGED

SEQ ID NO: 111 - protein, 673 aa, Bos taurus
MAKPAQGAKYRGSIRDFPDFNPSQDAETLYNAMKGFGSDKEAILELITSRSNRQRQEICQNYKSLYGKDLIADLKYELTGKFERLIVGLMRPPAYADAKEIKDAISGIGTDEKCLIEILASRTNEQIHQLVAAYKDAYERDLEADITGDTSGHFRKMLVVLLQGTREEDDVVSEDLVQQDVQDLYEAGELKWGTDEAQFIYILGNRSKQHLRLVFDEYLKTTGKPIEASIRGELSGDFEKLMLAVVKCIRSTAEYFAERLFKAMKGLGTRDNTLIRIMVSRSELDMLDIREIFRTKYEKSLYSMIKNDTSGEYKKTLLKLCGGDDDAAGQFFPEAAQVAYQMWELSAVARVELKGTVRPAGDFNPDADAKALRKAMKGLGTDEDTIIDIIAHRSNAQRQQIRQTFKSHFGRDLMADLKSELSGDLARLILGLMMPPAHYDAKQLKKAMEGAGTDEKALIEILATRTNAEIQAINKAYKEDYHKSLEDALSSDTSGHFKRILISLATGNREEGGEDRERAREDAQVAAEILEIADTTSGDKSSLETRFMMILCTRSYPHLRRVFQEFIKMTNYDVEHTIKKEMSGDVRDVFVAIVQSVKNKPLFFADKLYKSMKGAGTDEKTLTRIMVSRSEIDLLNIRREFIEKYDKSLHQAIEGDTSGHFLKALLAICGGED

SEQ ID NO: 112 - protein, 673 aa, Bubalus bubalis
MAKPAQGAKYRGSIRDFPNFNPSQDAETLYNAMKGFGSDKEAILELITSRSNRQRQEICQNYKSLYGKDLIADLKYELTGKFERLIVGLMRPPAYADAKEIKDAISGIGTDEKCLIEILASRTNKQIHQLVAAYKDAYERDLEADITGDTSGHFRKMLVVLLQGTREEDDVVSEDLVQQDVQDLYEAGELKWGTDEAQFIYILGNRSKQHLRLVFDGYLKTTGKPIEASIRGELSGDFEKLMLAVVKCIRSTAEYFAERLFKAMKGLGTRDNTLIRIMVSRSELDMLDIREIFRTKYEKSLYSMIKNDTSGEYKKTLLKLCGGDDDAAGQFFPEAAQVAYQMWELSAVARVELKGTVRPAGDFNPDADAKALRKAMKGLGTDEDTIIDIIAHRSNAQRQQIRQTFKSHFGRDLMADLKSELSGDLARLILGLMMPPAHYDAKQLKKAMEGAGTDEKALIEILATRTNAEIQAINEAYKEDYHKSLEDALSSDTSGHFKRILISLATGNREEGGEDRERAREDAQVAAEILEIADTTSGDKSSLETRFMMILCTRSYPHLRRVFQEFVKMTNYDVEHTIKKEMSGDVRDVFVAIVQSVKNKPLFFADKLYKSMKGAGTDEKTLTRIMVSRSEIDLLNIRREFVEKYDKSLHQAIEGDTSGHFLKALLAICGGED

SEQ ID NO: 113 - protein, 673 aa, Cervus elaphus
MAKPAQGAKYRGSIRDFPNFDPSQDAETLYNAMKGFGSDKEAILELITSRSNRQRQEICQNYKSLYGKDLIADLKYELTGKFERLIVGLMRPPAYGDAKEIKDAISGIGTDEKCLIEILASRTNEQIHQLVAAYKDAYERDLEADITGDTSGHFRKMLVVLLQGTREEDDVVSEDLVQQDVQDLYEAGELKWGTDEAQFIYILGNRSKQHLRLVFDEYLKTTGKPIEASIRGELSGDFEKLMLAVVKCIRSTAEYFAERLFKAMKGLGTRDNTLIRIMVSRSELDMLDIREIFRTKYEKSLYSMIKNDTSGEYKKALLKLCGGDDDAAGQFFPEAAQVAYQMWELSAVARVELKGTVHPAGDFNPDADAKALRKAMKGLGTDEDTIIDIIAHRSNAQRQQIRQTFKSHFGRDLMADLKSELSGDLARLILGLMMPPAHYDAKQLKKAMEGAGTDEKALIEILATRTNAEIQAINEAYKEDYHKSLEDALSSDTSGHFKRILISLATGNREEGGEDRERAREDAQVAAEILEIADTTSGDKSSLETRFMMILCTRSYPHLRRVFQEFVKMTNYDVEHTIKKEMSGDVRDVFVAIVQSVKNKPLFFADKLYKSMKGAGTDEKTLTRIMVSRSEIDLLNIRREFIEKYDKSLHQAIEGDTSGHFLKALLAICGGED

SEQ ID NO: 114 - protein, 667 aa, Cervus elaphus
MAKPAQGAKYRGSIRDFPNFDPSQDAETLYNAMKGFGSDKEAILELITSRSNRQRQEICQNYKSLYGKDLIADLKYELTGKFERLIVGLMRPPAYGDAKEIKDAISGIGTDEKCLIEILASRTNEQIHQLVAAYKDAYERDLEADITGDTSGHFRKMLVVLLQGTREEDDVVSEDLVQQDVQDLYEAGELKWGTDEAQFIYILGNRSKQHLRLVFDEYLKTTGKPIEASIRGELSGDFEKLMLAVVKCIRSTAEYFAERLFKAMKGLGTRDNTLIRIMVSRSELDMLDIREIFRTKYEKSLYSMIKNDTSGEYKKALLKLCGGDDDAAGQFFPEAAQVAYQMWELSAVARVELKGTVHPAGDFNPDADAKALRKAMKGLGTDEDTIIDIIAHRSNAQRQQIRQTFKSHFGRDLMADLKSELSGDLARLILGLMMPPAHYDAKQLKKAMEGAGTDEKALIEILATRTNAEIQAINEAYKEDYHKSLEDALSSDTSGHFKRILISLATGNREEGGEDRERAREDAQEIADTTSGDKSSLETRFMMILCTRSYPHLRRVFQEFVKMTNYDVEHTIKKEMSGDVRDVFVAIVQSVKNKPLFFADKLYKSMKGAGTDEKTLTRIMVSRSEIDLLNIRREFIEKYDKSLHQAIEGDTSGHFLKALLAICGGED

SEQ ID NO: 115 - protein, 671 aa, Gallus gallus
MAPKGKVYRGSVKDFPGFNASQDADALCNAMKGFGSDKDAILDLITSRSNKQRLEICQAYKSQYGKDLIADLKYELTGKFERLIVSLMRPPAYSDAKEIKDAIAGIGTDEKCLIEILASRTNQEIHDLVAAYKDAYERDLEADVVGDTSGHFKKMLVVLLQGAREEDDVVSEDLVEQDAKDLLEAGELKWGTDEAQFIYILGRRSKQHLRMVFDEYLKISGKPIERSIRAELSGDFEKLKLAVVKCVRSTAEYFAERLYKAMKGLGTRDNTLIHIMVSRSEIDMLDIREVFRTKYDKSLHNMIKEDTSGEYKKALLKLCEGDDDAAAEFFPEAAQVAYRMWELSAVAKVELRGTVQPASNFNDDGDAQVLRKAMKGLGTDEGAIIEVLTQRSNAQRQQILKAYKAHYGRDLLADLKSELSGSLANLILGLMLTPAQYDAKQLRKAVEGDGTDESTLVEIMATRNNQEIAAINEAYQQAYHKSLEDDLSSDTSVHFKRLLVSLALGNRDEGPENLTQAHEDAKVVAETLKLADVPSNDSSDSLETRFLSILCTRSYPHLRRVFQEFVKMTNHDVEHAIRKRMSGDVRDAFVAIVRSVKNKPAFFADKLYKSMKGAGTDERTLTRIMISRSEIDLLNIRGEFIDLFDKSLYQMIEKDSGDYCKALLALCGGDD

SEQ ID NO: 116 - protein, 672 aa, Meleagris gallopavo
MAPKGKVYRGSVKDFPGFNASQDADALYNAMKGFGSDKDAILDLITSRSNKQRVEICQAYKSQYGKDLIADLKYELTGKFERLIVSLMRPPAYSDAKEIKDAIAGVGTDEKCLIEILASRTNQEIHDLVAAYKDAYERDLEADIVGDTSGHFKKMLVVLLQGAREEDDVVSEDLVEQDAKDLLEAGELKWGTDEAQFIYILGRRSKQHLRMVFDEYMKISGKPIERSIRGELSGDFEKLMLAVVKCIRSTAEYFAERLYKAMKGLGTRDNTLIRIMVSRSEIDMLDIREVFRTKYEKSLHNMIKEDTSGEYKKALLKLCGGDDDAAGEFFPEAAQVAYRMWELSAVAKVELRGTVQPVGNFNDDGDAQVLRKAMKGLGTDEGAIIEVLTQRSNAQRQQILKAYKAHYGRDLLADLKSELSGSLAKLILGLMLTPAQYDAKQLRKAVEGAGTDESTLIEIMATRNNQEIAAINEAYQQAYHKSLEDDLSSDTSGHFKRILVSLALGNRDEGPENLTQAHEDAKVVAETLKLADVASNDSSDSLETRFLSILCTRSYPHLRRVFQEFIKMTNHDVEHAIRKRMSGDVRDAFVAIVRSVKNKPAFFADKLYKSMKGAGTDERTLTRIMISRSEIDLLNIRGEFIDLFDKSLYHMIEKDTSGDYRKALLALCGGDD

SEQ ID NO: 117 - protein, 666 aa, Meleagris gallopavo
MAPKGKVYRGSVKDFPGFNASQDADALYNAMKGFGSDKDAILDLITSRSNKQRVEICQAYKSQYGKDLIADLKYELTGKFERLIVSLMRPPAYSDAKEIKDAIAGVGTDEKCLIEILASRTNQEIHDLVAAYKDAYERDLEADIVGDTSGHFKKMLVVLLQGAREEDDVVSEDLVEQDAKDLLEAGELKWGTDEAQFIYILGRRSKQHLRMVFDEYMKISGKPIERSIRGELSGDFEKLMLAVVKCIRSTAEYFAERLYKAMKGLGTRDNTLIRIMVSRSEIDMLDIREVFRTKYEKSLHNMIKEDTSGEYKKALLKLCGGDDDAAGEFFPEAAQVAYRMWELSAVAKVELRGTVQPVGNFNDDGDAQVLRKAMKGLGTDEGAIIEVLTQRSNAQRQQILKAYKAHYGRDLLADLKSELSGSLAKLILGLMLTPAQYDAKQLRKAVEGAGTDESTLIEIMATRNNQEIAAINEAYQQAYHKSLEDDLSSDTSGHFKRILVSLALGNRDEGPENLTQAHEDAKKLADVASNDSSDSLETRFLSILCTRSYPHLRRVFQEFIKMTNHDVEHAIRKRMSGDVRDAFVAIVRSVKNKPAFFADKLYKSMKGAGTDERTLTRIMISRSEIDLLNIRGEFIDLFDKSLYHMIEKDTSGDYRKALLALCGGDD

SEQ ID NO: 118 - protein, 673 aa, Oryctolagus cuniculus
MAKPAQGAKYRGSVHDFPDFDPSQDAEALYTAMKGIGSDKEAILELITSRSNRQRQEVCQSYKSLYGKDLIADLKYELTGKFERLIVGLMRPLAYCDAKEIKDAVSGIGTDEKCLIEILASRTNEQIHQLVAAYKDAYERDLEADIIGDTSGHFQKMLVVLLQGTREEDDVVSEDLVQQDVQDLYEAGELKWGTDEAQFIYILGNRSKQHLRLVFDEYLKTTGKPIEASIRGELSGDFEKLMLAVVKCVRSTPEYFAERLFKAMKGLGTRDNTLIRIMVTRSELDMLDIREIFRTKYEKSLYSMIKNDTSGEYKKALLKLCGGDDDAAGQFFPEAAQVAYQMWELSAVARVELKGTVRPAGDFNPDADAKALRKAMKGIGTDEDTIIDIITHRSNAQRQQIRQTFKSHFGRDLMADLKSEISGDLARLILGLMMPPAHYDAKQLKKAMEGAGTDEKALIEILATRTNAEIHAINEAYKEDYHKSLEDALSSDTSGHFRRILISLATGNREEGGEDRDQAREDAQVAAEILEIADTPSGDKTSLETRFMTILCTRSYPHLRRVFQEFIKMTNYDVEHVIKKEMSGDVRDAFVAIVQSVKNKALFFADKLYKSMKGAGTDEKTLTRIMVSRSEIDLFNVRQEFIEKYDKSLHQAIEGDTSGDFRKALLSLCGGED

SEQ ID NO: 119 - protein, 673 aa, Oryctolagus cuniculus
MAKPAQGAKYRGSVHDFPDFDPSQDAEALYTAMKGIGGDKEAILELITSRSNRQRQEVCQSYKSLYGKDLIADLKYELTGKFERLIVGLMRPLAYCDAKEIKDAVSGIGTDEKCLIEILASRTNEQIHQLVAAYKDAYERDLEADIIGDTSGHFQKMLVVLLQGTREEDDVVSEDLVQQDVQDLYEAGELKWGTDEAQFIYILGNRSKQHLRLVFDEYLKTTGKPIEASIRGELSGDFEKLMLAVVKCVRSTPEYFAERLFKAMKGLGTRDNTLIRIMVTRSELDMLDIREIFRTKYEKSLYSMIKNDTSGEYKKALLKLCGGDDDAAGQFFPEAAQVAYQMWELSAVARVELKGTVRPAGDFNPDADAKALRKAMKGIGTDEDTIIDIITHRSNAQRQQIRQTFKSHFGRDLMADLKSEISGDLARLILGLMMPPAHYDAKQLKKAMEGAGTDEKALIEILATRTNAEIHAINEAYKEDYHKSLEDALSSDTSGHFRRILISLATGNREEGGEDRDQAREDAQVAAEILEIADTPSGDKTSLETRFMTILCTRSYPHLRRVFQEFIKMTNYDVEHVIKKEMSGDVRDAFVAIVQSVKNKALFFADKLYKSMKGAGTDEKTLTRIMVSRSEIDLFNVRQEFIEKYDKSLHQAIEGDTSGDFRKALLSLCGGED

SEQ ID NO: 120 - protein, 667 aa, Oryctolagus cuniculus
MAKPAQGAKYRGSVHDFPDFDPSQDAEALYTAMKGIGSDKEAILELITSRSNRQRQEVCQSYKSLYGKDLIADLKYELTGKFERLIVGLMRPLAYCDAKEIKDAVSGIGTDEKCLIEILASRTNEQIHQLVAAYKDAYERDLEADIIGDTSGHFQKMLVVLLQGTREEDDVVSEDLVQQDVQDLYEAGELKWGTDEAQFIYILGNRSKQHLRLVFDEYLKTTGKPIEASIRGELSGDFEKLMLAVVKCVRSTPEYFAERLFKAMKGLGTRDNTLIRIMVTRSELDMLDIREIFRTKYEKSLYSMIKNDTSGEYKKALLKLCGGDDDAAGQFFPEAAQVAYQMWELSAVARVELKGTVRPAGDFNPDADAKALRKAMKGIGTDEDTIIDIITHRSNAQRQQIRQTFKSHFGRDLMADLKSEISGDLARLILGLMMPPAHYDAKQLKKAMEGAGTDEKALIEILATRTNAEIHAINEAYKEDYHKSLEDALSSDTSGHFRRILISLATGNREEGGEDRDQAREDAQEIADTPSGDKTSLETRFMTILCTRSYPHLRRVFQEFIKMTNYDVEHVIKKEMSGDVRDAFVAIVQSVKNKALFFADKLYKSMKGAGTDEKTLTRIMVSRSEIDLFNVRQEFIEKYDKSLHQAIEGDTSGDFRKALLSLCGGED

SEQ ID NO: 121 - protein, 673 aa, Ovis aries
MAKPAQGAKYRGSVCDFPDFNPSQDAETLYNAMKGFGSDKEAILELIISRSNRQRQEICQNYKSLYGKDLIADLKYELTGKFERLIVGLMRPPAYADAKEIKDAISGIGTDEKCLIEILASRTNKQIHQLVAAYKDAYERDLEADITGDTSGHFRKMLVVLLQGTREEDDVVSEDLVQQDVQDLYEAGELKWGTDEAQFIYILGNRSKQHLRLVFDEYLKTTGKPIEASIRGELSGDFEKLMLAVVKCVRSTAEYFAERLFKAMKGLGTRDNTLIRIMVSRSELDMLDIREIFRTKYEKSLYSMIKNDTSGEYKKALLKLCGGDDDAAGQFFPEAAQVAYQMWELSAVARVELKGTVRPAGDFNPDADAKALRKAMKGLGTDEDTIIDIIAHRSNAQRQQIRQTFKSHFGRDLMADLKSELSGDLARLILGLMMPPAHYDAKQLKKAMEGAGTDEKALIEILATRTNAEIQAIKEAYKEDYHKSLEDALSSDTSGHFKRILISLATGNREEGGEDRERAREDAQVAAEILEMADTSSGDKSSLETRFMMILCTRSYPHLRRVFQEFVKMTNYDVEHTIKKEMSGDVRDVFVAIVQSVKNKPLFFADKLYKSMKGAGTDEKTLTRIMVSRSEIDLLNIRREFIEKYDKSLHQAIEGDTSGHFLKALLAICGGED

SEQ ID NO: 122 - protein, 667 aa, Ovis aries
MAKPAQGAKYRGSVCDFPDFNPSQDAETLYNAMKGFGSDKEAILELIISRSNRQRQEICQNYKSLYGKDLIADLKYELTGKFERLIVGLMRPPAYADAKEIKDAISGIGTDEKCLIEILASRTNKQIHQLVAAYKDAYERDLEADITGDTSGHFRKMLVVLLQGTREEDDVVSEDLVQQDVQDLYEAGELKWGTDEAQFIYILGNRSKQHLRLVFDEYLKTTGKPIEASIRGELSGDFEKLMLAVVKCVRSTAEYFAERLFKAMKGLGTRDNTLIRIMVSRSELDMLDIREIFRTKYEKSLYSMIKNDTSGEYKKALLKLCGGDDDAAGQFFPEAAQVAYQMWELSAVARVELKGTVRPAGDFNPDADAKALRKAMKGLGTDEDTIIDIIAHRSNAQRQQIRQTFKSHFGRDLMADLKSELSGDLARLILGLMMPPAHYDAKQLKKAMEGAGTDEKALIEILATRTNAEIQAIKEAYKEDYHKSLEDALSSDTSGHFKRILISLATGNREEGGEDRERAREDAQEMADTSSGDKSSLETRFMMILCTRSYPHLRRVFQEFVKMTNYDVEHTIKKEMSGDVRDVFVAIVQSVKNKPLFFADKLYKSMKGAGTDEKTLTRIMVSRSEIDLLNIRREFIEKYDKSLHQAIEGDTSGHFLKALLAICGGED

SEQ ID NO: 123 - protein, 641 aa, Ovis aries
MKGFGSDKEAILELIISRSNRQRQEICQNYKSLYGKDLIADLKYELTGKFERLIVGLMRPPAYADAKEIKDAISGIGTDEKCLIEILASRTNKQIHQLVAAYKDAYERDLEADITGDTSGHFRKMLVVLLQGTREEDDVVSEDLVQQDVQDLYEAGELKWGTDEAQFIYILGNRSKQHLRLVFDEYLKTTGKPIEASIRGELSGDFEKLMLAVVKCVRSTAEYFAERLFKAMKGLGTRDNTLIRIMVSRSELDMLDIREIFRTKYEKSLYSMIKNDTSGEYKKALLKLCGGDDDAAGQFFPEAAQVAYQMWELSAVARVELKGTVRPAGDFNPDADAKALRKAMKGLGTDEDTIIDIIAHRSNAQRQQIRQTFKSHFGRDLMADLKSELSGDLARLILGLMMPPAHYDAKQLKKAMEGAGTDEKALIEILATRTNAEIQAIKEAYKEDYHKSLEDALSSDTSGHFKRILISLATGNREEGGEDRERAREDAQVAAEILEMADTSSGDKSSLETRFMMILCTRSYPHLRRVFQEFVKMTNYDVEHTIKKEMSGDVRDVFVAIVQSVKNKPLFFADKLYKSMKGAGTDEKTLTRIMVSRSEIDLLNIRREFIEKYDKSLHQAIEGDTSGHFLKALLAICGGED

SEQ ID NO: 124 - protein, 662 aa, Salmo salar
MGMGFRGTVTDAPDFDASADAETLYNAMKGIGSDKEAILDLVTGRSNAQRQEIVQAYKSSYGKDLIDDLKYELTGNFERLIVSLMRPQAYHDAKEIHDAIKGAGTDEKCLIEVLASRNNQQIHDLVEAYTDAYGSDIEEDVTGETSGHFKKMLVVLLQGTRDEPGVVHADLIEEDAQVLFAAGEEQWGTEESIFIMLLGNRSFNHLQMVFDKYQEIAEKSIEDSIKSELSGDFERLMLAVVQCIRSVPMYFAKRLYKSMKGLGTQDNTLIRIMICRSEIDMLDIRECFRMCYEKSLYNMIKEDTSGDYKRTLLALCGGDDDLAGEFFPEAAQLAYKMWETSAMTKVQLRPTIRPASDFDPADDAQNLRKAMKGFGTDEDVIIDIVANRSNEQRQEIRQAFKSILGRDLMKDLKSELSKNLERLIIGLMLTPAEFDAKMMRKAMEGAGTDEHALIEILVTRSNEEIHAMNAAYQDGYKKSMEEAIQSDTSGRFSQILTSLVQGAREQGPADWDRALVDAQELADACNEDSDDMEIKFMSILCTRSFPHLRRVFQEFVRCSNKDIEQIIKKEMSGDVKQAMYGIVRSVKNQPNYIAERLYKAMKCIGTDDRALIRIMVSRSEVDLFNIRKEFKETHDCSLHEFIQGDTSGDYRKTLLMLCGGQD

SEQ ID NO: 125 - protein, 673 aa, Sus scrofa
  VARIANT 516: note = Any amino acid
MAKPAQGARYRGSIHDFPNFDPSQDAETLYNAMKGFGSDKEAILELITSRSNRQRQEICQNYKSLYGKDLIADLKYELTGKFERLIVGLMRPPAYGDAKEIKDAVSGIGTDEKCLTEILASRTNEQIHQLVAAYKDAYERDLEADVIGDTSGHFQKMLVVLLQGTREEDDVVSEDLVHQDVQDLYEAGELKWGTDEAQFIYILGNRSKQHLRLVFDEYLKTTGKPIEASIRGELSGDFEKLMLAVVKCIRSTPEYFAERLFKAMKGLGTRDNTLIRIMVSRSELDMLDIREIFRTKYEKSLYSMIKNDTSGEYKKALLKLCGGDDDAAGQFFPEAAQVAYQMWELSAVARVELKGTVHPAGDFNPDADAKALRKAMKGLGTDEDTIIDIVTRRSNAQRQQIRQTFKSHFGRDLMVDLKSELSGDLARLILGLMMPPAHYDAKQLKKAMEGAGTDEKALIEILATRTNAEIRAINEAYKEDYHKSLEDALSSDTSGHFKRILISLATGNREEGGEDXDPAQEDAKVAAEILEIADTSSGDKTSLETRFMTILCTRSYQHLRRVFQEFVKMTNYDVEHTIKKEMSGDVRDVFVAIVQSVKNKPLFFADKLYKSMKGAGTDEKTLTRIMISRSEIDLLNIRREFIEKYDKSLHQAIEGDTSGHFLKALLAICGGED

SEQ ID NO: 126 - protein, 665 aa, Thunnus albacares
MVFRGTITDAPDFDPSADAETLYNAMKGIGSDKEAILDLVTSRSNAQRQEIIAAYKSNFGQDLIDDLKYELTGKFERLIVSLMRTPAYHDAKEINDAVKGAGTNERCLIEIMASRNNKQIHEMVEAYKDAYGTDIEEDIIGDTSGHFKKMLIVLLQGTRDESGVVDGDLAEQDAQDLYAAGEEQWGTDEAKFIMILGNRSVTHLRMVFDEYEKIAEMSIEDSIKNELSGDFERLMLAVVQCIRSVPMFFAKRLYKSMKGLGTADNTLIRIMISRSEQDMLDIRECFRLNYEKSLYNMIKDDTSGDYKRTLLNLCGGDDDLAGEFFPEAAQIAYKMWEISSMTKVKLRPTVRPASDFDAGADAQALRKAMKGFGTDEDAIIDIIAHRSNAQRQEIRQAFKSLLGRDLMKDLKSELSKNLERLIIGLMLTPAEFDAKMMRKAMEGAGTDEHALIEILVTRSNEEIHAMNAAYEEGYKKSLEEAIQSDTSGHFCRILVSLVQGAREEGPADLERANADAQELADACNADSDDMEMKFMSILCTRSFPHLRKVFQEFVKCSNKDIEQIIKKEMSGDVKQAFYAIVRSVKNQPSYFADRLYKAMKGLGTDDRALIRIMVSRSEIDLFNIRKEFKETQDVSLHEFIQVETMIGDTSGDYRKTLLELCGGED

SEQ ID NO: 127 - protein, 660 aa, Thunnus albacares
MVFRGTITDAPDFDPSADAETLYNAMKGIGSDKEAILDLVTSRSNAQRQEIIAAYKSNFGQDLIDDLKYELTGKFERLIVSLMRTPAYHDAKEINDAVKGAGTNERCLIEIMASRNNKQIHEMVEAYKDAYGTDIEEDIIGDTSGHFKKMLIVLLQGTRDESGVVDGDLAEQDAQDLYAAGEEQWGTDEAKFIMILGNRSVTHLRMVFDEYEKIAEMSIEDSIKNELSGDFERLMLAVVQCIRSVPMFFAKRLYKSMKGLGTADNTLIRIMISRSEQDMLDIRECFRLNYEKSLYNMIKDDTSGDYKRTLLNLCGGDDDLAGEFFPEAAQIAYKMWEISSMTKVKLRPTVRPASDFDAGADAQALRKAMKGFGTDEDAIIDIIAHRSNAQRQEIRQAFKSLLGRDLMKDLKSELSKNLERLIIGLMLTPAEFDAKMMRKAMEGAGTDEHALIEILVTRSNEEIHAMNAAYEEGYKKSLEEAIQSDTSGHFCRILVSLVQGAREEGPADLERANADAQELADACNADSDDMEMKFMSILCTRSFPHLRKVFQEFVKCSNKDIEQIIKKEMSGDVKQAFYAIVRSVKNQPSYFADRLYKAMKGLGTDDRALIRIMVSRSEIDLFNIRKEFKETQDVSLHEFIQGDTSGDYRKTLLELCGGED

SEQ ID NO: 128 - protein, 452 aa, Anas platyrhynchos
MSYPGYPPSGYPGFPGYPPTGQESVYPPAGQYTYPAGYPPAGGGTYPAAPPNAGYPGAAGYPAPGGYPGAPQAGGMPSYPGAPAGPGFGVPPAGPAFGGYPQPPVQSYGGGGPAQIPDYSGGQAPSPMPGLPAATVQYTQGTIQAAPNFDANRDAEILRKAMKGFGTDEQAIINVVANRSNDQRQKIKAAFKTMYGKDLIKDLKSELSGNVEELILALFMPSTYYDAWSLRHAMKGVGTQERVLIEILCTRTNQEIRDIVNCYKSEFGRDIEQDIRADTSGHFERLLVSMCQGNRDENQNVDYQKAQEDAQRLYQAGEGKLGTDESCFNMVLASRSFPQLKATVEAYSRIANRDLLSSIDREFSGNVERGLKAILQCALNRPAFFAERLYYSMKGAGTDDSTLIRIIVTRSEIDLVQIKQVFSQMYQKTLASMISSDTSGDYRRLLLAIVGQ

SEQ ID NO: 129 - protein, 377 aa, Anas platyrhynchos
MPSYPGAPAGPGFGVPPAGPAFGGYPQPPVQSYGGGGPAQIPDYSGGQAPSPMPGLPAATVQYTQGTIQAAPNFDANRDAEILRKAMKGFGTDEQAIINVVANRSNDQRQKIKAAFKTMYGKDLIKDLKSELSGNVEELILALFMPSTYYDAWSLRHAMKGVGTQERVLIEILCTRTNQEIRDIVNCYKSEFGRDIEQDIRADTSGHFERLLVSMCQGNRDENQNVDYQKAQEDAQRLYQAGEGKLGTDESCFNMVLASRSFPQLKATVEAYSRIANRDLLSSIDREFSGNVERGLKAILQCALNRPAFFAERLYYSMKGAGTDDSTLIRIIVTRSEIDLVQIKQVFSQMYQKTLASMISSDTSGDYRRLLLAIVGQ

SEQ ID NO: 130 - protein, 463 aa, Bos taurus
MSYPGYPPTGYPPFPGYPPTGQESSFPPPGQYPYPSGFPPMGGGAYPPAPSSGYPGAGGYPAPGGYPAPGGYPGAPQPGGAPSYPGGQGFGAPPGGAGFPGYPQPPTQSYGGGPAQVPLPGGFPGGAMPSQYPGGQSPYPSQPAPMTQGTHGTIRPAANFDAMRDAEVLRKAMKGFGTDEQAIIDVVANRSNDQRQKIKAAFKTMYGKDLIKDLKSELSGNMEELILALFMPSTYYDAWSLRNAMKGAGTQERVLIEILCTRTNQEIREIVRCYQSEFGRDLEKDIRSDTSGHFERLLVSMCQGNRDENQNVNHQLAQEDAQRLYQAGEGRLGTDESCFNMILATRSFPQLKATMEAYSRMANRDLLNSVSREFSGNVESGLKTILQCALNRPAFFAERLYYSMKGAGTDDSTLVRIVVTRSEIDLVQIKQMFSQMYQKTLGTMIASDTSGDYRKLLLAIVGQ

SEQ ID NO: 131 - protein, 485 aa, Cervus elaphus
MSYPGYPPTGYPPFPGYPPTGQESSFPPPGQYPYPSGFPPMGGGAYPPAPSGGYPGAGGYPAPGGYPAPGGYPGAPHPGGAPSYPGGQGFGAPPGGAGFPGYPQPPTQSYGGGPAQVPLPGGFPGGAMPSQYPGGQSPYPSQINTESFPSYPVFSPVSLDYSSEPAAMTQGTHGTIRPAANFDAMRDAEVLRKAMKGFGTDEQAIIDVVANRSNDQRQKIKAAFKTMYGKDLIKDLKSELSGNMEELILALFMPPTYYDAWSLRNAMKGAGTQERVLIEILCTRTNQEIREIVRCYQSEFGRDLEKDIRSDTSGHFERLLVSMCQGNRDENQNVNHQLAQDDAQRLYQAGEGRLGTDESCFNMVLATRSFPQLKATMEAYSRMANRDLLSSVSREFSGNVESGLKTILQCALNRPAFFAERLYYSMKGAGTDDSTLVRIVVTRSEIDLVQIKQMFSQMYQKTLGTVIASDTSGDYQKLLLAIVGQ

SEQ ID NO: 132 - protein, 463 aa, Cervus elaphus
MSYPGYPPTGYPPFPGYPPTGQESSFPPPGQYPYPSGFPPMGGGAYPPAPSGGYPGAGGYPAPGGYPAPGGYPGAPHPGGAPSYPGGQGFGAPPGGAGFPGYPQPPTQSYGGGPAQVPLPGGFPGGAMPSQYPGGQSPYPSQPAAMTQGTHGTIRPAANFDAMRDAEVLRKAMKGFGTDEQAIIDVVANRSNDQRQKIKAAFKTMYGKDLIKDLKSELSGNMEELILALFMPPTYYDAWSLRNAMKGAGTQERVLIEILCTRTNQEIREIVRCYQSEFGRDLEKDIRSDTSGHFERLLVSMCQGNRDENQNVNHQLAQDDAQRLYQAGEGRLGTDESCFNMVLATRSFPQLKATMEAYSRMANRDLLSSVSREFSGNVESGLKTILQCALNRPAFFAERLYYSMKGAGTDDSTLVRIVVTRSEIDLVQIKQMFSQMYQKTLGTVIASDTSGDYQKLLLAIVGQ

SEQ ID NO: 133 - protein, 459 aa, Gallus gallus
MAYPGYPPSGYPAFPGYPPTGQESVYPPAGQYTYPAAPGGYPPAGGGSFPAAPPSTGYPEAGGYPAPGGYPAPGGFPGAPQAGGMPSYPGGPGYGVPPTGPGFGGYPQPPAQSYAGGGPAQIPVGYPGGQAPSPMPGPPAAMVQYTQGTIQAAPNFDAGRDAEILRKAMKGIGTDEKAIIDVVSNRSNDQRQKIKAAFKTMYGKDLIKDLKSELSGNVEELILALFMPRTYYDAWSLRHAMKGAGTQERVLIEILCTRTNQEIREIVNCYKSEFGRDIEQDIRADTSGHFERLLVSMCQGNRDENQTVDYQKAQEDAQRLYQAGEGKLGTDESCFNMVLASRSFPQLRATAEAYSRIANRDLASSIDREFSGNVERGLKAILQCAFDRPAFFAERLYYAMKGAGTDDSTLIRIIVTRSEIDLVQIKQKFAEMYQKTLATMIASDTSGDYRRLLLAIVGQ

SEQ ID NO: 134 - protein, 459 aa, Meleagris gallopavo
MSYPGYPPSGYPAFPGYPPTGQESVYPPAGQYTYPAAPGGYPPAGGGSFPAAPPSAGYPEAGGYPAPGGFPAPGGFPGAPQAGGMPSYPGGPGFGMPPTGPGFGGYPQPPAQSYGGGGPAQIPVGYPGGQAPSPMPGPPAAMVQYTQGTIQAAPNFDAGRDAEILRKAMKGFGTDEKAIIDVVSNRSNDQRQKIKAAFKTMYGKDLIKDLKSELSGNVEELILALFMPRTYYDAWSLRHAMKGAGTQERVLIEILCTRTNQEIREIVNCYKSEFGRDIEQDIRADTSGHFERLLVSMCQGNRDENQTVDYQKAQEDAQRLYQAGEGKLGTDESCFNMVLASRSFPQLRATAEAYSRIANRDLASSIDREFSGNVERGLKAILQCAFDRPAFFAERLYHAMRGAGTDDSTLIRIVVTRSEIDLVQIKQKFTEMYQKTLATMIASDTSGDYRRLLLAIVGQ

SEQ ID NO: 135 - protein, 364 aa, Meleagris gallopavo
MPPTGPGFGGYPQPPAQSYGGGGPAQIPVGYPGGQAPSPMPGPPAAMVQYTQGTIQAAPNFDAGRDAEILRKAMKGFGTDEKAIIDVVSNRSNDQRQKIKAAFKTMYGKDLIKDLKSELSGNVEELILALFMPRTYYDAWSLRHAMKGAGTQERVLIEILCTRTNQEIREIVNCYKSEFGRDIEQDIRADTSGHFERLLVSMCQGNRDENQTVDYQKAQEDAQRLYQAGEGKLGTDESCFNMVLASRSFPQLRATAEAYSRIANRDLASSIDREFSGNVERGLKAILQCAFDRPAFFAERLYHAMRGAGTDDSTLIRIVVTRSEIDLVQIKQKFTEMYQKTLATMIASDTSGDYRRLLLAIVGQ

SEQ ID NO: 136 - protein, 591 aa, Oryctolagus cuniculus
MAPRSRRRRHKKPPSSVVSTIVTPPADVTSVPLTLSKPGPSIDALGFFSLENNVPGLSQLILQKLNMKSYEEYKLVVDGGTPVSGFGFRCPQEMFQKMEDTFRFCAHCRVLPSGLPDTKVLRHCKRMSYPGYPPSGYPPFPGYPPAGQESSFPPPGQYPYPSGFPPKGGGAYPPAPSGGYPGSGGYPAPGGYPTPGGYPGAPQPGGAPSYPGVPPGQGFGAPPGGAGYPGYPQPPSQSYGGGPAQVPLPGGFPGGQPSQYPGGQAPYPSQPAAVTQGTQGTIHPAANFDAMKDAEILRKAMKGFGTDEQAIVDVVANRSNDQRQKIKAAFKTMYGKDLIKDLKSELSGNMEELILALFMPPTYYDAWSLRNAMQGAGTQERVLIEILCTRTNQEIREIVRCYQSEFGRDLEKDIRSDTSGHFERLLVSMCQGNRDENQTVNHQMAQEDAQRLYQAGEGKLGTDESCFNMILATRSFPQLRATMEAYSRMANRDLLASVSREFSGYIESGLKTILQCALNRPAFFAERLYYSMKGAGTDDSTLVRIVVTRSEVDLVQIKQMFTQMYQKTLGTMIASDTSGDYRKLLLGIVGQ

SEQ ID NO: 137 - protein, 613 aa, Oryctolagus cuniculus
MAPRSRRRRHKKPPSSVVSTIVTPPADVTSVPLTLSKPGPSIDALGFFSLENNVPGLSQLILQKLNMKSYEEYKLVVDGGTPVSGFGFRCPQEMFQKMEDTFRFCAHCRVLPSGLPDTKVLRHCKRMSYPGYPPSGYPPFPGYPPAGQESSFPPPGQYPYPSGFPPKGGGAYPPAPSGGYPGSGGYPAPGGYPTPGGYPGAPQPGGAPSYPGVPPGQGFGAPPGGAGYPGYPQPPSQSYGGGPAQVPLPGGFPGGQPSQYPGGQAPYPSQISTESFSSYPVFSPVSLDYSSDPAAVTQGTQGTIHPAANFDAMKDAEILRKAMKGFGTDEQAIVDVVANRSNDQRQKIKAAFKTMYGKDLIKDLKSELSGNMEELILALFMPPTYYDAWSLRNAMQGAGTQERVLIEILCTRTNQEIREIVRCYQSEFGRDLEKDIRSDTSGHFERLLVSMCQGNRDENQTVNHQMAQEDAQRLYQAGEGKLGTDESCFNMILATRSFPQLRATMEAYSRMANRDLLASVSREFSGYIESGLKTILQCALNRPAFFAERLYYSMKGAGTDDSTLVRIVVTRSEVDLVQIKQMFTQMYQKTLGTMIASDTSGDYRKLLLGIVGQ

SEQ ID NO: 138 - protein, 487 aa, Oryctolagus cuniculus
MSYPGYPPSGYPPFPGYPPAGQESSFPPPGQYPYPSGFPPKGGGAYPPAPSGGYPGSGGYPAPGGYPTPGGYPGAPQPGGAPSYPGVPPGQGFGAPPGGAGYPGYPQPPSQSYGGGPAQVPLPGGFPGGQPSQYPGGQAPYPSQISTESFSSYPVFSPVSLDYSSDPAAVTQGTQGTIHPAANFDAMKDAEILRKAMKGFGTDEQAIVDVVANRSNDQRQKIKAAFKTMYGKDLIKDLKSELSGNMEELILALFMPPTYYDAWSLRNAMQGAGTQERVLIEILCTRTNQEIREIVRCYQSEFGRDLEKDIRSDTSGHFERLLVSMCQGNRDENQTVNHQMAQEDAQRLYQAGEGKLGTDESCFNMILATRSFPQLRATMEAYSRMANRDLLASVSREFSGYIESGLKTILQCALNRPAFFAERLYYSMKGAGTDDSTLVRIVVTRSEVDLVQIKQMFTQMYQKTLGTMIASDTSGDYRKLLLGIVGQ

SEQ ID NO: 139 - protein, 485 aa, Ovis aries
MSYPGYPPTGYPPFPGYPPTGQESSFPPPGQYPYPSGFPPMGGGAYPPAPSSGYPGAGGYPAPGGYPAPGGYPGAPQPGGAPSYPGGQGFGAPPGGAGFSGYPQPPTQSYGGGPAQVPLPGGFPGGAMPSQYPGGQPPYPSQINTESFPSYPVFSPVSWDYSSEPAAMTQGTHGTIRPAANFDAMRDAEVLRKAMKGFGTDEQAIIDVVANRSNDQRQKIKAAFKTMYGKDLIKDLKSELSGNMEELILALFMPPTYYDAWSLRNAMKGAGTQERVLIEILCSRTNQEIREIVRCYQSEFGRDLEKDIRSDTSGHFERLLVSMCQGNRDENQNVNHQLAQEDAQRLYQAGEGRLGTDESCFNMVLATRSFPQLKATMEAYSRMANRDLLSSVSREFSGNVESGLKTILQCALNRPAFFAERLYYSMKGAGTDDSTLVRIVVTRSEIDLVQIKQMFSQMYQKTLGTVIASDTSGDYRKLLLAIVGQ

SEQ ID NO: 140 - protein, 470 aa, Ovis aries
MSYPGYPPTGYPPFPGYPPTGQESSFPPPGQYPYPSGFPPMGGGAYPPAPSSGYPGAGGYPAPGGYPAPGGYPGAPQPGGAPSYPGGQGFGAPPGGAGFSGYPQPPTQSYGGGPAQVPLPGGFPGGAMPSQYPGGQPPYPSQINTESFPSYPVFSPVSWDYSSEPAAMTQGTHGTIRPAANFDAMRDAEVLRKAMKGFGTDEQAIIDVVANRSNDQRQKIKAAFKTMYGKDLIKDLKSELSGNMEELILALFMPPTYYDAWSLRNAMKGAGTQERVLIEILCSRTNQEIREIVRCYQSEFGRDLEKDIRSDTSGHFERLLVSMCQGNRDENQNVNHQLAQEDAQRLYQAGEGRLGTDESCFNMVLATRSFPQLKATMEAYSRMANRDLLSSVSREFSGNVESGLKTILQCALNRPAFFAERLYYSMKGAGTDDSTLVRIVVTRSEWLHHFTLLPAVNRDFNFSTSWPTLC

SEQ ID NO: 141 - protein, 463 aa, Ovis aries
MSYPGYPPTGYPPFPGYPPTGQESSFPPPGQYPYPSGFPPMGGGAYPPAPSSGYPGAGGYPAPGGYPAPGGYPGAPQPGGAPSYPGGQGFGAPPGGAGFSGYPQPPTQSYGGGPAQVPLPGGFPGGAMPSQYPGGQPPYPSQPAAMTQGTHGTIRPAANFDAMRDAEVLRKAMKGFGTDEQAIIDVVANRSNDQRQKIKAAFKTMYGKDLIKDLKSELSGNMEELILALFMPPTYYDAWSLRNAMKGAGTQERVLIEILCSRTNQEIREIVRCYQSEFGRDLEKDIRSDTSGHFERLLVSMCQGNRDENQNVNHQLAQEDAQRLYQAGEGRLGTDESCFNMVLATRSFPQLKATMEAYSRMANRDLLSSVSREFSGNVESGLKTILQCALNRPAFFAERLYYSMKGAGTDDSTLVRIVVTRSEIDLVQIKQMFSQMYQKTLGTVIASDTSGDYRKLLLAIVGQ

SEQ ID NO: 142 - protein, 188 aa, Salmo salar
MDPNKMPNAPPPGWNPDEKSGMGQPAPPPYQDHPQYPNAGYPAPAGYQAPAGYPPNPQGYAPPPQYGGAPGAPYSQPYHQGQYPPGQYPQSTVTVQPTVFVTHGALPYPLPDYLGYSIFTMLCCCLPLGIAALIYSISTRDANNQGHQQIAEKNSRLARILNHTALGIGITVIVLYIVYAIILGNRIN

SEQ ID NO: 143 - protein, 482 aa, Sus scrofa
MSYPGYTPTGYPPFPGYPPAGQESSFPPPGQYPYPSGFPPMGGGAYPPAPSSGYPGAGGYPAPGGYPAPGGYPGAPQPGGAPSYPGGQGFGAPPGGAGFSGYPQPPSQSYGGGPAQVPVPGGFPGGPMPSQYPGGQTPYPSQISTESFPSYPVFSPISLDYSSELATVAQGTIRPAANFDAMRDAEILRKAMKGFGTDEQAIIDVVANRSNDQRQKIKAAFKTMYGKDLIKDLKSELSGNMEELILALFMPSTYYDAWSLRNAMKGAGTQERVLIEILCTRTNQEIREIVRCYQSEFGRDLEKDIRSDTSGHFERLLVSMCQGNRDENQNVNPQMAQEDAQRLYQAGEGRLGTDESCFNMILATRSFPQLKATMEAYSRMANRDLLSSVGREFSGNVESGLKTILQCALNRPAFFAERLYYSMKGAGTDDSTLVRIVVTRSEIDLVQIKQMFSQMYQKTLGTMIASDTSGDYRRLLLAIVGQ

SEQ ID NO: 144 - protein, 460 aa, Sus scrofa
MSYPGYTPTGYPPFPGYPPAGQESSFPPPGQYPYPSGFPPMGGGAYPPAPSSGYPGAGGYPAPGGYPAPGGYPGAPQPGGAPSYPGGQGFGAPPGGAGFSGYPQPPSQSYGGGPAQVPVPGGFPGGPMPSQYPGGQTPYPSQLATVAQGTIRPAANFDAMRDAEILRKAMKGFGTDEQAIIDVVANRSNDQRQKIKAAFKTMYGKDLIKDLKSELSGNMEELILALFMPSTYYDAWSLRNAMKGAGTQERVLIEILCTRTNQEIREIVRCYQSEFGRDLEKDIRSDTSGHFERLLVSMCQGNRDENQNVNPQMAQEDAQRLYQAGEGRLGTDESCFNMILATRSFPQLKATMEAYSRMANRDLLSSVGREFSGNVESGLKTILQCALNRPAFFAERLYYSMKGAGTDDSTLVRIVVTRSEIDLVQIKQMFSQMYQKTLGTMIASDTSGDYRRLLLAIVGQ

SEQ ID NO: 145 - protein, 326 aa, Anas platyrhynchos
MAWWKAWSEAEGVSVTGALNFDPVPDAQTLYKAMKGLGTDEQAIIDVLTKRSNMQRQQIAKSFKGQFGKDLIESLKSELSGDFERLIVALMYPPYKYDAKELYDAMKGVGTSEGVIIEILASRTKAQIREIIKAYKEEYGSDLEEDIKSETSGYFEQILVCLLQGERDNATLYVDTALALQDAQTLYAAGEKIRGTDEIQFITILCTRSATHLMKVFEEYQKLAGKSIEDSIRSETSGSLEEAMLAIVKCTRNVHCYFAERLYHALKGAGTRDGTLIRVIVSRSEVDLNLIKAEFKRIAGQSLSSMIVDDTSGDYKTALLNLCGSD

SEQ ID NO: 146 - protein, 327 aa, Bos taurus
MAWWKAWVEQEGVSVKGSPHFNPDPDAETLYKAMKGIGTNEQAIIDVLTKRSNAQRQQIAKSFKAQFGKDLIETLKSELSGKFERLIIALMYPPYRYEAKELYDAMKGIGTKEGVIIEILASRTKNQLQEIMKAYEEDYGSNLEEDIKADTSGYLERILVCLLQGSRDDLSGYVDPGLALQDAQDLYAAGEKICGTDEMKFITILCTRSATHLMRVFEEYEKIANKSIEDSIKSETHGSLEEAMLTVVKCTRNLHGYFAERLYFAMKGAGTLDGTLIRNIVSRSEIDLNLIKNQFKKMYGKTLSSMIMEDTSGDYKNALLNLVGSDL

SEQ ID NO: 147 - protein, 327 aa, Bubalus bubalis
MAWWKAWVEQEGVSVKGSPHFNPDPDAETLYKAMKGIGTNEQAIIDVLTKRSNAQRQQIAKSFKAQFGKDLIETLKSELSGKFERLIIALMYPPYRYEAKELYDAMKGIGTKEGVIIEILASRTKNQLREIMKAYEEDYGSNLEEDIKADTSGYLERILVCLLQGSRDDLSGYVDPGLALQDAQDLYAAGEKICGTDEMKFITILCTRSATHLMRVFEEYEKIANKSIEDSIKSETHGSLEEAMLTVVKCTRNLHSYFAERLYFAMKGAGTMDGTLIRNIVSRSEIDLNLIKNQFKKMYGKTLSSMIMEDTSGDYKNALLNLVGSDL

SEQ ID NO: 148 - protein, 327 aa, Cervus elaphus
MAWWKAWVEQEGVSVKGSPHFNPDPDAETLYKAMKGIGTNEQAIIDVLTKRSNAQRQQIAKSFKAQFGKDLIETLKSELSGKFERLIIALMYPPYRYEAKELYDAMKGIGTKEGVIIEILASRTKNQLQEIMKAYEEDYGSNLEEDIKADTSGYLERILVCLLQGSRDDLSGYVDPGLALQDAQDLYAAGEKICGTDEMKFITILCTRSATHLMRVFEEYEKIANKSIEDSIKSETHGSLEEAMLTVVKCTRNLHSYFAERLYYAMKGAGTLDGTLIRNIVSRSEIDLNLIKNQFKKMYGKTLSSMIMEDTSGDYKNALLNLVGSDL

SEQ ID NO: 149 - protein, 327 aa, Gallus gallus
MAWWKAWSEEEGKPVIGVYSNFDPTPDAQTLYKAMKGLGTDEQAIIEVLTKRSNKQRQEIAKSFKAQFGKDLIDSLRSELSGNFERLIVALMYPPYKYEAKELYDAMKGVGTSEDVIIEILASRTKAQIKEIIKAYKEEYGSDLEHDIASETSGYFKQILVCLLQGERDNASLYVDTALARQDAETLFSAGEKIKGTDEIQFITILCKRSATHLLKVFEEYQKLASKSIEDSIKSETKGSLEDAMLAIVRCTRNIRQYFAERLYHALKGAGTDDGTLIRVIVSRNEVDLNLIKDEFRRIAGQPLSSMIVDDTSGDYKTALLNLCGSD

SEQ ID NO: 150 - protein, 329 aa, Meleagris gallopavo
MGQPLQLLLSEEEGKPVIGVYSNFDPAPDAQTLYKAMKGLGTDEQAIIEVLTKRSNKQRQEIAKSFKAQFGKDLIDSLRSELSGDFERLMVALMYPPYKYEAKELFDAMKGVGTSEDVIIEILASRTKAQIKEIIKAYKEEYGSDLEQDIASETSGYFKQILVCLLQGERDNASLYVDTALARQDAEALFSAGEKIKGTDEIQFITILCKRSATHLLKVFEEYQKLAGKSIEDSIKSETKGSLEDAMLAIVRCTRNIRQYFAERLYHALKGAGTDDGTLIRVIVSRNEVDLNLIKDEFKRIAGQPLSSMIVDDTSGDYKTALLNLCGSD

SEQ ID NO: 151 - protein, 327 aa, Meleagris gallopavo
MAWWKAWSEEEGKPVIGVYSNFDPAPDAQTLYKAMKGLGTDEQAIIEVLTKRSNKQRQEIAKSFKAQFGKDLIDSLRSELSGDFERLMVALMYPPYKYEAKELFDAMKGVGTSEDVIIEILASRTKAQIKEIIKAYKEEYGSDLEQDIASETSGYFKQILVCLLQGERDNASLYVDTALARQDAEALFSAGEKIKGTDEIQFITILCKRSATHLLKVFEEYQKLAGKSIEDSIKSETKGSLEDAMLAIVRCTRNIRQYFAERLYHALKGAGTDDGTLIRVIVSRNEVDLNLIKDEFKRIAGQPLSSMIVDDTSGDYKTALLNLCGSD

SEQ ID NO: 152 - protein, 293 aa, Meleagris gallopavo
MKGLGTDEQAIIEVLTKRSNKQRQEIAKSFKAQFGKDLIDSLRSELSGDFERLMVALMYPPYKYEAKELFDAMKGVGTSEDVIIEILASRTKAQIKEIIKAYKEEYGSDLEQDIASETSGYFKQILVCLLQGERDNASLYVDTALARQDAEALFSAGEKIKGTDEIQFITILCKRSATHLLKVFEEYQKLAGKSIEDSIKSETKGSLEDAMLAIVRCTRNIRQYFAERLYHALKGAGTDDGTLIRVIVSRNEVDLNLIKDEFKRIAGQPLSSMIVDDTSGDYKTALLNLCGSD

SEQ ID NO: 153 - protein, 327 aa, Oryctolagus cuniculus
MAWWKAWVEQEGVTVKGSPHFNPVPDAETLYKAMKGIGTNEQAIIDVLTRRSSAQRQQIAKSFKAQFGSDLTETLKSELSGKFERLIVALMYPPYRYEAKELHDAMKGLGTKEGVIIEILASRTKNQLQEIMKAYEEDYGSSLEEDIQADTSGYLERILVCLLQGSRDDVTGFVDPGLALQDAQDLYAAGEKICGTDEMKFITILCTRSARHLMRVFEEYEKIANKSIEDSIKSETHGSLEEAMLTIVKCTRNLHCYFAERLHYAMKGAGTLDGTLIRNIVSRSEIDLNLIKGHYKKMYGKTLSSMIMEDTSGDYKNALLSLVGSDP

SEQ ID NO: 154 - protein, 327 aa, Ovis aries
MAWWKAWVEQEGVSVKGSPHFNPDPDAETLYKAMKGIGTNEQAIIDVLTKRSNAQRQQIAKSFKAQFGKDLIETLKSELSGKFERLIIALMYPPYRYEAKELYDAMKGIGTKEGVIIEILASRTKNQLQEIMKAYEEDYGSNLEEDIKADTSGYLERILVCLLQGSRDDLSGYVDPGLALQDAQDLYAAGEKICGTDEMKFITILCTRSATHLLRVFEEYEKIANKSIEDSIKSETHGSLEEAMLTVVKCTRNLHSYFAERLYFAMKGAGTLDGTLIRNIVSRSEIDLNLIKNQFKKMYGKTLSSMIMEDTSGDYKNALLNLVGSDL

SEQ ID NO: 155 - protein, 327 aa, Sus scrofa
MAWWKAWIEQEGVSVKGSPHFNPDPDAETLYKAMKGIGTNEQAIIDVLTKRSNAQRQQIAKSFKAQFGKDLTETLKSELSGKFERLIIALMYPPYKYEAKELHDAMEGLGTKEGVIIEILASRTKNQLQEIMKAYEEDYGSSLEEDIQADTSGYLERILVCLLQGSRDDLSGFVDPGLALQDAQDLYAAGEKIRGTDEMKFITILCTRSATHLMRVFEEYEKIANKSIEDSIKSETHGSLEEAMLTVVKCTRNLHSYFAERLYYAMKGAGTRDGTLIRNIVSRSEIDLNLIKGQFNKMYGKTLSSMIMEDTSGDYKNALLNLVGSDP

SEQ ID NO: 156 - protein, 345 aa, Bos taurus
MSVTHGKMGLSLTQEILSHLGLANKTAAWGTLGTLRTFLSFSADKDVQRLLKAIAGQGVDRIAILDVLTNRSREQRQLISRAFHERTQQDLLKSLQAALSGNLERIVVALLQPAAHLDARELRTALKGSGSAEDVALEILATRTPPQLQECLAVYKHNFQVDAAEDIKSETRGILRDLLLALAKGGREAYTGIIDYNLAAQDVQALKQAEGPSTERTWVLVFTQRNPEHLVRVLNQYQWYTGHGLEKTVRARFHGAACVALLNLASVIRNTPLYFADKLHQALQETEPNYQALMRILISRSETDLLSIRAEFRKKFGKSLYSSLQDAVKGDCRSALLALCRAEDL

SEQ ID NO: 157 - protein, 492 aa, Cervus elaphus
MSVTHGKMGLSLTQEILSHLGLANKTAAWGTLGTLRTFLSFSVDKDVQRLLKAIAGQGVDCIAILDVLTNRSREQRQLISRAFHERTQQDLLKSLQAALSGNLERIVVALLQPAAHLDARELRKALKGSGSAEDVALEILATRTPPQLRECLAVYKHSKGVEDGALRAGQDSPVSPALPAGSPVGACLPLVPLGSLLLPHRRAQSPQGDSQLSSLKREKQSLTVVVWGSRVDPEVMTRLSPVTVPRAPKSPSQPLADSAITPHPRLNGRSMQPGGKVGTCSRRCQFSAPGCCPCSDCHMPHPLIDFQVDAAEDIKSETRGILRDLLLALAKGGREAYTGIIDYNLAAQDVQALKQAEGPSTERMWVLVFTQRNSEHLVRVLNQYQRDTGRELEKTVRARFHGAACVALLSLALVIRNTPLYFADKLHQALQETEPNYQVLMRILISRSETDLLSIRAEFRKKYGKSLYSSLQDAVKGDCRSALLALCRAEDL

SEQ ID NO: 158 - protein, 454 aa, Cervus elaphus
MSVTHGKMGLSLTQEILSHLGLANKTAAWGTLGTLRTFLSFSVDKDVQRLLKAIAGQGVDCIAILDVLTNRSREQRQLISRAFHERTQQGSGSAEDVALEILATRTPPQLRECLAVYKHSKGVEDGALRAGQDSPVSPALPAGSPVGACLPLVPLGSLLLPHRRAQSPQGDSQLSSLKREKQSLTVVVWGSRVDPEVMTRLSPVTVPRAPKSPSQPLADSAITPHPRLNGRSMQPGGKVGTCSRRCQFSAPGCCPCSDCHMPHPLIDFQVDAAEDIKSETRGILRDLLLALAKGGREAYTGIIDYNLAAQDVQALKQAEGPSTERMWVLVFTQRNSEHLVRVLNQYQRDTGRELEKTVRARFHGAACVALLSLALVIRNTPLYFADKLHQALQETEPNYQVLMRILISRSETDLLSIRAEFRKKYGKSLYSSLQDAVKGDCRSALLALCRAEDL

SEQ ID NO: 159 - protein, 345 aa, Cervus elaphus
MSVTHGKMGLSLTQEILSHLGLANKTAAWGTLGTLRTFLSFSVDKDVQRLLKAIAGQGVDCIAILDVLTNRSREQRQLISRAFHERTQQDLLKSLQAALSGNLERIVVALLQPAAHLDARELRKALKGSGSAEDVALEILATRTPPQLRECLAVYKHNFQVDAAEDIKSETRGILRDLLLALAKGGREAYTGIIDYNLAAQDVQALKQAEGPSTERMWVLVFTQRNSEHLVRVLNQYQRDTGRELEKTVRARFHGAACVALLSLALVIRNTPLYFADKLHQALQETEPNYQVLMRILISRSETDLLSIRAEFRKKYGKSLYSSLQDAVKGDCRSALLALCRAEDL

SEQ ID NO: 160 - protein, 307 aa, Cervus elaphus
MSVTHGKMGLSLTQEILSHLGLANKTAAWGTLGTLRTFLSFSVDKDVQRLLKAIAGQGVDCIAILDVLTNRSREQRQLISRAFHERTQQGSGSAEDVALEILATRTPPQLRECLAVYKHNFQVDAAEDIKSETRGILRDLLLALAKGGREAYTGIIDYNLAAQDVQALKQAEGPSTERMWVLVFTQRNSEHLVRVLNQYQRDTGRELEKTVRARFHGAACVALLSLALVIRNTPLYFADKLHQALQETEPNYQVLMRILISRSETDLLSIRAEFRKKYGKSLYSSLQDAVKGDCRSALLALCRAEDL

SEQ ID NO: 161 - protein, 105 aa, Oryctolagus cuniculus
MEEAKRGHTGAATRPLHQHRLTPRTSGLARSTMSASSGKPAPTLTQEILSHLGLASKTAAWGTLGVLRTFLSFSVDKDVQRLLRAIAGQGEPRPSALGEPANGRS

SEQ ID NO: 162 - protein, 345 aa, Ovis aries
MSVTHGKMGLSLTQEILSHLGLANKTAAWGTLGTLRTFLSFNVDKDVQRLLKAIAGQGVDRIAILDVLTNRSREQRQLISRAFHERTQQDLLKSLQAALSGNLERIVVALLQPAAHLDARELRTALKGPGSAEDVALEILATRTPPQLQECLAVYKHNFQVDAAEDIKSETRGILRDLLLALAKGGREAYTGIIDYNLAAQDVQALKQAEGPSTERTWVLVFTQRNPEHLIRVLNQYQWYTGHELEKTVRARFHGAACVALLNLALVIRNTPMYFADKLHQALQETEPNYQALMRILISRSETDLLSIRAEFRKKFGKSLYSSLQDAVKGDCRSALLALCRAEDL

SEQ ID NO: 163 - protein, 365 aa, Sus scrofa
MSVTSGKTGASLTEEILNHLGLADKTAAWGTLGTLGTLRTFLSFSVDKDVQRLLRAIAGQGVDHNAIVDVLTNRSREQRQLISRAFQERTQQDLLKSLQAALSGNLERIVVALLHPAAHLDARELRTALKVPGEEDSGSAEDVAMEILATRTPPQLQECLAVYRHDFKVEAEEDIKCATKGILRDLLLALAKGGRESYAGIIDYNLAEQDVQALKRAEGPSTEQTWVFILTQRNPEHLVRVLHQYQRSTGHELEKTIRDRFHGAAQVALLSLASVILNAALYFADKLHQALQETEPNYQVLMRILISRSETDLLSIRAEFKKKFGKSLYSSLQVRLAWFLTWIPRASPVTSTLHTAEHIHALENS

SEQ ID NO: 164 - protein, 360 aa, Sus scrofa
MSVTSGKTGASLTEEILNHLGLADKTAAWGTLGTLGTLRTFLSFSVDKDVQRLLRAIAGQGVDHNAIVDVLTNRSREQRQLISRAFQERTQQDLLKSLQAALSGNLERIVVALLHPAAHLDARELRTALKDSGSAEDVAMEILATRTPPQLQECLAVYRHDFKVEAEEDIKCATKGILRDLLLALAKGGRESYAGIIDYNLAEQDVQALKRAEGPSTEQTWVFILTQRNPEHLVRVLHQYQRSTGHELEKTIRDRFHGAAQVALLSLASVILNAALYFADKLHQALQETEPNYQVLMRILISRSETDLLSIRAEFKKKFGKSLYSSLQVRLAWFLTWIPRASPVTSTLHTAEHIHALENS

SEQ ID NO: 165 - protein, 353 aa, Sus scrofa
MSVTSGKTGASLTEEILNHLGLADKTAAWGTLGTLGTLRTFLSFSVDKDVQRLLRAIAGQGVDHNAIVDVLTNRSREQRQLISRAFQERTQQDLLKSLQAALSGNLERIVVALLHPAAHLDARELRTALKVPGEEDSGSAEDVAMEILATRTPPQLQECLAVYRHDFKVEAEEDIKCATKGILRDLLLALAKGGRESYAGIIDYNLAEQDVQALKRAEGPSTEQTWVFILTQRNPEHLVRVLHQYQRSTGHELEKTIRDRFHGAAQVALLSLASVILNAALYFADKLHQALQETEPNYQVLMRILISRSETDLLSIRAEFKKKFGKSLYSSLQDTVKGDCRSALLALCRAEDL

SEQ ID NO: 166 - protein, 348 aa, Sus scrofa
MSVTSGKTGASLTEEILNHLGLADKTAAWGTLGTLGTLRTFLSFSVDKDVQRLLRAIAGQGVDHNAIVDVLTNRSREQRQLISRAFQERTQQDLLKSLQAALSGNLERIVVALLHPAAHLDARELRTALKDSGSAEDVAMEILATRTPPQLQECLAVYRHDFKVEAEEDIKCATKGILRDLLLALAKGGRESYAGIIDYNLAEQDVQALKRAEGPSTEQTWVFILTQRNPEHLVRVLHQYQRSTGHELEKTIRDRFHGAAQVALLSLASVILNAALYFADKLHQALQETEPNYQVLMRILISRSETDLLSIRAEFKKKFGKSLYSSLQDTVKGDCRSALLALCRAEDL

SEQ ID NO: 167 - protein, 336 aa, Sus scrofa
MSVTSGKTGASLTEEILNHLGLADKTAAWGTLGTLGTLRTFLSFSVDKDVQRLLRAIAGQGVDHNAIVDVLTNRSREQRQLISRAFQERTQQDLLKSLQAALSGNLERIVVALLHPAAHLDARELRTALKVPGEEDSGSAEDVAMEILATRTPPQLQECLAVYRHDFKVEAEEDIKCATKGILRDLLLALAKGGRESYAGIIDYNLAEQDVQALKRAEGPSTEQTWVFILTQRNPEHLVRVLHQYQRSTGHELEKTIRDRFHGAAQVALLSLASVILNAALYFADKLHQALQETEPNYQVLMRILISRSETDLLSIRAEFKKKFGKSLYSSLQVEK

SEQ ID NO: 168 - protein, 310 aa, Sus scrofa
MSVTSGKTGASLTEEILNHLGLADKTAAWGTLGTLGTLRTFLSFSVDKDVQRLLRAIAGQGVDHNAIVDVLTNRSREQRQLISRAFQERTQQDLLKSLQAALSGNLERIVVALLHPAAHLDARELRTALKVPGEEDSGSAEDVAMEILATRTPPQLQECLAVYRHDFKVEAEEDIKCATKGILRDLLLALAKGGRESYAGIIDYNLAEQDVQALKRAEGPSTEQTWVFILTQRNPEHLVRVLHQYQRSTGHELEKTIRDRFHGAAQVALLSLASVILNAALYFADKLHQALQVYKLSCQEPRTELWLELN

SEQ ID NO: 169 - protein, 298 aa, Sus scrofa
MSVTSGKTGASLTEEILNHLGLADKTAAWGTLGTLGTLRTFLSFSVDKDVQRLLRAIAGQGVDHNAIVDVLTNRSREQRQLISRAFQERTQQDLLKSLQAALSGNLERIVVALLHPAAHLDARELRTALKVPGEEDSGSAEDVAMEILATRTPPQLQECLAVYRHDFKVEAEEDIKCATKGILRDLLLALAKGGRESYAGIIDYNLAEQDVQALKRAEGPSTEQTWVFILTQRNPEHLVRVLHQYQRSTGHELEKTIRDRFHGAAQVALLSLASVILNAALYFADKLHQALQAASAFS

SEQ ID NO: 170 - protein, 375 aa, Gallus gallus
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 171 - protein, 434 aa, Gallus gallus
MSIQKIHAREILDSRGEPTVEVDLHTAKGHFRAAVPSGASTGIHEALEPRDGDKKRFLGKGVLKAVEHINKTIGPALIEKKISVVEQEKIDKVMIEMDGTENKSKFGANAILGVSLAVCKAGAAEKGVPLYRHIADLAGNTELILPVPAFNVINGGSHAGNKLAMQEFMVLPVGAASFHDAMRVGAEVYHSLKGVIKAKYGKDATNVGGEGGFAPNILDNHEALELLKAAIAQAGYTDKVVIGMDVAASEFCRDGRYHLDFKSPPHTKRYITGEQLGEIYRGFIKDYPVVSIEDPFDQDDWEAWKRFVFHVDIQVVGDDLTVTNPKRIAHGAEQHACNCLLLKVNQIGSVTESIQACKLAQSHGWGVMVSHRSGETEDTFIADLVVGLCTGQIKTGAPCRSERLAKYNQLMRIEEALGDKAKFAGRKFRNPKAK

SEQ ID NO: 172 - protein, 151 aa, Gallus gallus
MAEEQRELEERIIIKDQHTKEIDLVNRDPKRINEDVVKVDFEDVIAEPVGTYSFDGVWKGSYTTFTVSKYWCYRLLSAVLGIPLAVVWGFLFALISFCHIWAVVPCIKSYLIEIQCVSRIYSLCIHTFCDPLFEALSKICSNVRVMLRKET

SEQ ID NO: 173 - protein, 137 aa, Anas platyrhynchos
MPNFAGTWKMRSSENFDELLKALGVNAMLRKVAVAAASKPHVEIRQDGDQFYIKTSTTVRTTEINFKIGESFEEETVDGRKCRSLATWENENKIYCKQTLIEGDGPKTYWTRELANDELILTFGADDVVCTRIYVRE

SEQ ID NO: 174 - protein, 137 aa, Bos taurus
MPNFAGTWKMRSSENFDELLKALGVNAMLRKVAVAAASKPHVEIRQDGDQFYIKTSTTVRTTEINFKVGEGFEEETVDGRKCRSLPTWENENKIHCTQTLLEGDGPKTYWTRELANDELILTFGADDVVCTRIYVRE

SEQ ID NO: 175 - protein, 138 aa, Bos taurus
MPNFAGTWKMRSSENFDELLKALGVNAMLRKVAVAAASKPHVEIRQDGDQFYIKTSTTVRTTEINFKVGEGFEEETVDGRKCRSLPTWENENKIHCTQTLLEGDGPKTYWTRELANDDELILTFGADDVVCTRIYVRE

SEQ ID NO: 176 - protein, 137 aa, Bos taurus
MPNFAGTWKMRSSENFDELLKALGVNAMLRKVAVAAASKPHVEIRQDGDQFYIKTSTTVRTTEINFKVGEGFEEETVDGRKCRSLPTWENENKIHCTQTLLEGDGPKTYWTRELANDDELILTFGADDVVCTRIYVR

SEQ ID NO: 177 - protein, 135 aa, Bos taurus
MPPNLTGYYRFVSQKNLEDYLQALNVNMALRKIALLLKPDKEIDQRGNHMTVKTLSTFRNYVLEFEVGVEFEEDLRTVDGRKCQTIVTWEEEQLVCVQKGEVPNRGWRLWLEEEMLYQEVTARDAVCQCVFRKVK

SEQ ID NO: 178 - protein, 137 aa, Cervus elaphus; identical to SEQ ID NO: 174

SEQ ID NO: 179 - protein, 137 aa, Gallus gallus; identical to SEQ ID NO: 173

SEQ ID NO: 180 - protein, 137 aa, Oryctolagus cuniculus; identical to SEQ ID NO: 174

SEQ ID NO: 181 - protein, 139 aa, Salmo salar
MTNFAGTWKMRSSENFDELLKALGVNAMLRKVAGAAASKPHVEIRQNGEQFYIKTSTTVRTTEINFLIGQEFNEETVDGRKCKSLATWETESKMYCRQTLLDGNGPRTFWTRELRGDELILTFGADDVVCTRIYVRENK

SEQ ID NO: 182 - protein, 137 aa, Sus scrofa
MPNFAGTWKMRSSENFDELLKALGVNAMLRKVAVAAASKPHVEIRQDGDQFYIKTSTTVRTTEINFKVGEGFEEETVDGRKCRSLATWENENKIHCTQTLLEGDGPKTYWTRELANDELILTFGADDVVCTRIYVRE

SEQ ID NO: 183 - protein, 137 aa, Thunnus albacares
MPNFAGNWKMKSSENFDELLKALGVNAMLRKVAVAAASKPHVEIKQNGENFYIKTSTTVRTTEVNFVVGEEFNEETVDGRKCKSLATWETENKIYCKQTLLNGNGPKTFWSRELRGDELILTFGADDVVCTRIYVRA

SEQ ID NO: 184 - protein, 138 aa, Bos taurus
MPNFSGNWKIIRSENFEDLLKVLGVNVMLRKIAVAAASKPAVEIKQEGDTFYIKTSTTVRTTEINFKIGEEFEEQTVDGRPCKSLVKWESENKMVCEQRLLKGEGPKTSWTRELTNDGELILTMTADDIVCTRVYVRE

SEQ ID NO: 185 - protein, 138 aa, Bos taurus
MPNFSGNWKIIRSENFEDLLKVLGVNVMLRKIAVAAASKPAVEIKQEGDTFYIKTSTTVRTTEINFKIGEEFEEQTVDGRPCKSLVKWESENKMVCEQRLLKGEGPKTSWTRELTNDGELILTMTADDIVCTRVYIRE

SEQ ID NO: 186 - protein, 138 aa, Cervus elaphus; identical to SEQ ID NO: 184

SEQ ID NO: 187 - protein, 138 aa, Gallus gallus
MPNFSGNWKMKSSENFEELLKALGVNMMLRKIAVAAASKPAVEIKQDGESFYIKTSTTVRTTEISFRIGEEFEEQTVDGRPCKSLAKWESENKMVCEQRLLKGEGPRTGWSRELTNDGELILTMTADDVVCTRVYIRE

SEQ ID NO: 188 - protein, 138 aa, Meleagris gallopavo; identical to SEQ ID NO: 187

SEQ ID NO: 189 - protein, 138 aa, Oryctolagus cuniculus
MSNFSGNWKIIRSENFEELLKALGVNMMLRKIAVAAASKPTVEIKQDGDNFYIKTSTTVRTTEINFKIAEEFEDQTVDGRPCKSLVKWERENKMVCEQRLLKGDGPRTTWTRELTNDGELILTMTADDVVCTRVYVRE

SEQ ID NO: 190 - protein, 142 aa, Salmo salar
MERKIPDFAGTWKMKSSENFEELLKALGVNVMLRKIAVAAASKPSVEITQEGETLSIRTFTSVRTTHVTFTVGESFNEATVDGRPCTSLPKWETDSKISCEQTLQKGEGPKTAWTREITNDGELILTMSADDVVCTRVYVRE

SEQ ID NO: 191 - protein, 154 aa, Salmo salar
MIDCYNKLFTSEMDRQIANFSGMWKMKTSENFEELLKALGVNVFVRKIAVAAASRPAVEITQQGESLSIQTSTSIRTTHVSFTVGQSFNEDTVDGRPCTSTPRWETDSKISCEQTLQKGEGPKTAWTREVTNDGELILTMSAGDVVCTRVYQRE

SEQ ID NO: 192 - protein, 138 aa, Sus scrofa
MPNFSGNWKIIRSENFEDLLKVLGVNVMLRKIAVAAASKPAVEIKQDGDTFYIKTSTTVRTTEINFKIGEEFEEQTVDGRPCKSLVKWESEDKMVCEQRLLKGEGPKTSWTRELTNDGELILTMTADDIVCTRVYVRE

SEQ ID NO: 193 - protein, 135 aa, Bos taurus
MPVDFTGYWKMLANENFEEYLRALDVNVALRKIANLLKPDKEIVQEGDHMIIRTLSTFRNYIMDFQVGKEFEEDLTGIDDRKCMTTVSWDGDKLECVQKGEKEGRGWTQWIEGDELHLEMRVEGVVCKQVFKKVN

SEQ ID NO: 194 - protein, 135 aa, Sus scrofa
MPVDFTGYWKMLANENFEEYLRALDVNVALRKIANLLKPDKEIVQDGNHMIIRTLSTFRNYIMDFEVGKEFEEDLTGIDDRKCMTTVSWDGDKLECVQKGEKEGRGWTQWIEGDELHLEMRVQGVACKQVFKKVN

SEQ ID NO: 195 - protein, 134 aa, Sus scrofa
MTRDQNGTWEMESNDNFEGYMKALDIDFATRKIAVALTQTKIIEQDGDKFKTKTNSTFRNYDLDFTVGVEFDEYTKGLDNRNVKTLIIWEGDALVCVQKGEKENRGWKQWVEGDKLYLELTCGDQVCRQVFKKK

SEQ ID NO: 196 - protein, 135 aa, Sus scrofa
MPPNLTGYYRFVSQKNLEDYLQALNINLALRKIALLLKPDKEIDHRGNHMTVKTLSTFRNYVLEFEVGVEFEEDLGIVDGRKCQTIVTWEEEQLVCVQKGEVPNRGWRHWLEGETLYLEMTARDAVCEQVFRKVK

SEQ ID NO: 197 - protein, 134 aa, Sus scrofa
MPADLSGTWNLVSSDNFEGYMLALGIDFATRKIAKLLKPQKVIEQNGDSFTIHTNSSLRNYLVKFKVGEEFDEDNKGLDNRKCKSLVTWDNDRLTCVQKGEKKNRGWTHWIEGDELHLEMFCEGQVCKQTFQRA

SEQ ID NO: 198 - protein, 166 aa, Bos taurus
MASGVTVNDEVIKVFNDMKVRKSSTQEEIKKRKKAVLFCLSDDKRQIIVEEAKQILVGDIGDTVEDPYTSFVKLLPLNDCRYALYDATYETKESKKEDLVFIFWAPESAPLKSKMIYASSKDAIKKKFTGIKHEWQVNGLDDIKDRSTLGEKLGGNVVVSLEGKPL

SEQ ID NO: 199 - protein, 166 aa, Gallus gallus
MASGVTVNDEVIKVFNDMKVRKSSTPEEIKKRKKAVLFCLSDDKKQIIVEEATRILVGDIGDTVEDPYTAFVKLLPLNDCRYALYDATYETKESKKEDLVFIFWAPESAPLKSKMIYASSKDAIKKKFTGIKHEWQVNGLDDIKDRSTLGEKLGGNVVVSLEGKPL

SEQ ID NO: 200 - protein, 149 aa, Ovis aries
MKVRKSSTQEEIKKRKKAVLFCLSDDKRQIIVEEAKQILVGDIGDTVEDPYTSFVKLLPLNDCRYALYDATYETKESKKEDLVFIFWAPESAPLKSKMIYASSKDAIKKKFTGIKHEWQVNGLDDIKDRSTLGEKLGGNVVVSLEGKPL

SEQ ID NO: 201 - protein, 217 aa, Gallus gallus
MAGAKEVRKEPEKTPVPTVIIATDKAKEQERISTAREEISARHEQVHVSHEQIRREDVKPSVPKVVITTDKPKAPVLISKSKEGIATKKEHVSISHEKIKKEAKKTTVVPKIIAPVTVSRTREEITAKQEQMHLAYDQIEAGKRAEAVATVVAAVDQARVRSPWETEQVDETYVKKKTLEYGYKEHAVKDHEAQAEHHVATKEVKTVYVPPEKHIPA

SEQ ID NO: 202 - protein, 607 aa, Gallus gallus
MVRGGRRGGYGGLPPPHPFPVALLPAKCHRPIFSGGAMCVCAAGGGRWLHDAALCAKYTLPATQHRGGHVRHNSAVPPPPQTPRAPRAPCSIGMCVGMVAAPLRLIVPVSPPGVADPRLRPRAQHHGAGGDAGGALQARGHHLLAPHGPQRAAERRWGSPQVPWDPMVVNPKSPPGTSGSHSKSHWGSTGVTPNPHWEPIAVTSVPTGDQQWYPRVLLGTNDSHPKSPLGTHSGHQRGDPVPGVPAGCDNLVILWNVGTGEMLLALEDMHTDLIYNVGWNRNGSLLVTTCKDKKVRVIDPRKQTVVAEITKPHDGARPIRAIFMADGKIFTTGFSKMSERQLGLWDLERFAPHEGLRPVRAIFTREGHIFTTGFTRMSQRELGLWDPKNFEEPIALQEMDTSNGVLLPFYDADSSIVYLCGKGDSSIRYFEITDEAPYVHYLNTYSSKEPQRGMGFMPKRGLDVSKCEIARFFKLHERKCEPIVMTVPRKSDLFQDDLYPDTPGPEPALEADEWLSGKDAEPILISLRDGYVPVKNRELKVVKKNILDSKPPPGPRRSHSTSNTDISTPALDEVLEEIRVLKETVQAQEKRISALEHKLCQFTNGTD

SEQ ID NO: 203 - protein, 381 aa, Gallus gallus
MPFSSTHNKHKLKFSAEEEFPDLSKHNNHMAKVLTPELYKRLRDKETPSGFTLDDVIQTGVDNPGHPFIMTVGCVAGDEESYEVFKDLFDPVIQDRHGGYKPTDKHRTDLNHENLKGGDDLDPKYVLSSRVRTGRSIKGYSLPPHCSRGERRAVEKLSVEALNSLEGEFKGRYYPLKAMTEQEQQQLIDDHFLFDKPVSPLLLASGMARDWPDARGIWHNDNKTFLVWVNEEDHLRVISMEKGGNMKEVFRRFCVGLKKIEEIFKKAGHPFMWTEHLGYILTCPSNLGTGLRGGVHVKLPKLSQHPKFEEILHRLRLQKRGTGGVDTAAVGAVFDISNADRLGFSEVEQVQMVVDGVKLMVEMEKKLEQNQPIDDMIPAQK

SEQ ID NO: 204 - protein, 194 aa, Bos taurus
MPNWGGGAKCGACEKTVYHAEEIQCNGRSFHKTCFHCMACRKALDSTTVAAHESEIYCKVCYGRRYGPKGIGYGQGAGCLSTDTGEHLGLQFQQSPKQARSATTSSNPSKFAKFGESEKCPRCGKSVYAAEKVMGGGKPWHKTCFRCAICGKSLESTNVTDKDGELYCKVCYAKNFGPTGIGFGGLTHQVEKKD

SEQ ID NO: 205 - protein, 464 aa, Gallus gallus
MSQSYSSSQRVSSYRRTFGGGTSPVFPRASFGSRGSGSSVTSRVYQVSRTSAVPTLSTFRTTRVTPLRTYGSAYQGAGELLDFSLADAMNQEFLQTRTNEKVELQELNDRFANYIEKVRFLEQQNALMVAEVNRLRGKQPTRVAEMYEEELRELRRQVDALTGQRARVEVERDNLLDNLQKLKQKLQEEIQLKQEAENNLAAFRADVDAATLARIDLERRIESLQEEIAFLKKVHEEEIRELQAQLQEQHIQVEMDISKPDLTAALRDIRAQYESIAAKNIAEAEEWYKSKVSDLTQAANKNNDALRQAKQEMLEYRHQIQSYTCEIDALKGTNDSLMRQMREMEERFAGEAGGYQDTIARLEEEIRHLKDEMARHLREYQDLLNVKMALDVEIATYRKLLEGEENRISIPMHQTFASALNFRETSPDQRGSEVHTKKTVMIKTIETRDGEVVSEATQQQHEVL

SEQ ID NO: 206 - protein, 165 aa, Gallus gallus
MASGVQVADEVCRIFYDMKVRKCSTPEEVKKRKKAVIFCLSPDKKCIIVEEGKEILVGDVGVTVTDPFKHFVEMLPEKDCRYALYDASFETKESKKEELMFFLWAPEQAPLKSKMIYASSKDAIKKKFQGIKHECQANGPEDLNRACIAEKLGGSLVVAFEGSPV

SEQ ID NO: 207 - protein, 180 aa, Ovis aries
MLMRIAFLKMKSSEYLASGVQVADEVCRIFYDMKVRKCSTPEEIKKRKKAVIFCLSADKKCIIVEEGKEILVGDVGVTITDPFKHFVGMLPEKDCRYALYDASFETKESRKEELMFFLWAPELAPLKSKMIYASSKDAIKKKFQGIKHECQANGPEDLNRACIAEKLGGSLIVAFEGCPV

SEQ ID NO: 208 - protein, 244 aa, Gallus gallus
MSAHVLWYEEVEDDYEREDVQKKTFTKWINAQFAKCGRRCIEDLFNDFRDGRKLLELLECLTGQKIAKEKGSTRVHALNNVNKALQILQRNNVDLVNIGSSDIVDGNHKLTLGLIWNIILHWQVKDVMKNIMAGLQQTNSEKILLSWVRQSTRNYPQVNVINFTSSWSDGLAFNALLHSHRPDLFDWNAVASQQSPVQRLDHAFNIARQHLGIEKLLDPEDVATACPDKKSILMYVTSLFQVLP

SEQ ID NO: 209 - protein, 490 aa, Gallus gallus
MTANGTAEPVQIQFGLINCGNKYLTAEAFGFKVNASAASMKKKQIWTLEQDGEDSSAVLLKSHLGRYLAADKDGRVSCDSEEPGPDCRFLVVAHGDGRWSLQSEPHRRFFGGTEDRLSCFAPSVSPAEKWSVHLAMHPQANLYSLARKRYAHLGPRRDELAVDRDVPWGVDALITLLFVEQRYSLQSCDHRLLRADGRLVPSAEPGTAFTLEFRCGKVAFRDGEGRYLAPSGPSGTLKAGKSSKVGKDELFALEQSCPQVVLRAGNDRNVSTRQGMDLSANQDEEGDQETYQLEINKDTKKCAFRTYTGKYWTLTSNGGIQSTASTKNASCYFDIEWCDKRITLRAANGKYVTAKKNGQLAASMETAGETEHFVMKLINRPIIVLRGEHGFIGCRKVTGTLDSNRSSYDVFQLEFNDGAYNIKDTTGKYWMVGSESSVTSSSDTPVDFFFEFCDYNKVAIKINGKYLKGDHAGVLKASADAIDASTLWEY

SEQ ID NO: 210 - protein, 127 aa, Bos taurus
MNFSGKYQVQTQENYEAFMKAVGMPDDIIQKGKDIKGVSEIVQNGKHFKFIITAGSKVIQNEFTLGEECEMEFMTGEKIKAVVQQEGDNKLVTTFKGIKSVTEFNGDTVTSTMTKGDVVFKRVSKRI

SEQ ID NO: 211 - protein, 126 aa, Gallus gallus [sequence omitted]

SEQ ID NO: 212 - protein, 127 aa, Sus scrofa
MNFSGKYQVQSQENFEAFMKAVGLPDELIQKGKDIKGTSEIVQNGKHFKLTITTGSKVVQNEFTLGEECEMETLTGEKVKTVVQLEGDNKLVTTFKGIKSVTELNGDIITSTMTLGDIVFKRISKRI

SEQ ID NO: 213 - protein, 140 aa, Bubalus bubalis
MVDLFQGTWKSISCENFEEYMKELGIGRASRKLGCLAKPTVTIMTKEDLITIKTKSIFKNNEISFKLGEEFEETTPSGHKTKSMVILDDDSLVQVQVWDGKEITIRRKLVDEKMVVESVVNDVTCTRTYERVQTNPVSNS

SEQ ID NO: 214 - protein, 140 aa, Cervus elaphus
MVELFQGTWKSISCENFEEYMKELGIGRASRKLGCLAKPTVTIMTQEDLITIKTKSIFKNNEISFKLGEEFEETTPGGVKTESMVILDDDSLIQVQDWDGKEITIRRKLVDEKMVVESVVNNVTCTRIYERVQTSPVSNF

SEQ ID NO: 215 - protein, 132 aa, Oryctolagus cuniculus
MVDQLQGTWKSVSCENFEEYMKELGIGKASRKLGSLAKPTVTISTNGDVITIKTKSIFKNNKISFKLGEEFEETTPGGRKTKSTVTLDNDAMIHTQDWGDKETTIRRKLVDGKMVVESAVNNVICTRTYERV

SEQ ID NO: 216 - protein, 152 aa, Ovis aries
MMQFFLKANLSRMVDLFQGTWKSISCENFEEYMKELGIGRASRKLGCLAKPTVTIITKEDLITIKTKSIFKNNEISFKLGEEFEETTPGGHKTKSTVILDDGSLIQVQDWDGKEITIRRKLVDEKLVVESVVNNVTCTRTYERVQTNPVSNS

SEQ ID NO: 217 - protein, 132 aa, Bos taurus
MAFDGTWKVDRNENYEKFMEKMGINVVKRKLAAHDNLKLIITQEGNKFTVKESSTFRSIEIIFELGVTFNYSLADGTELSGAWALEGDKLVGKFKRLDNGNALNTVREIIGGEMVQTYTYEGVEAKRIFKKE

SEQ ID NO: 218 - protein, 132 aa, Sus scrofa
MAFDGAWKIDRNENYDKFMEKMGINVVKRKLAAHDNLKLIITQEGNKFTVKESSTFRNIEIVFELGVTFNYSLADGTELTGNWNLEGNKLVGKFQRVDNGKELNTVREIIGDEMVQTYVYEGVEAKRIFKKN

SEQ ID NO: 219 - protein, 133 aa, Bos taurus
MVDAFVGTWKLVDSKNFDDYMKSLGVGFATRQVGNMTKPTTIIEVNGDTVIIKTQSTFKNTEISFKLGVEFDETTADDRKVKSIVTLDGGKLVHVQKWNGQETSLVREMVDGKLILTLTHGTAVCTRTYEKQA

SEQ ID NO: 220 - protein, 98 aa, Bos taurus
MTKPTTIIEVNGDTVIIKTQSTFKNTEISFKLGVEFDETTADDRKVKSIVTLDGGKLVHVQKWNGQETSLVREMVDGKLILTLTHGTAVCTRTYEKQA

SEQ ID NO: 221 - protein, 133 aa, Sus scrofa
MVDAFAGTWKLVDSKNFDDYMKSIGVGFATRQVANMTKPTTIIEVNGDTIIIKTQSTFKSTEISFKLGVEFDETTADDRKVKSIVTLDGGKLVHLQKWNGQETTLVRELVDGKLILTLTHGSAVCTRTYEKEA

SEQ ID NO: 222 - protein, 132 aa, Anas platyrhynchos
MCDHFVGTWKLLSSENFEDYMKELGVGFATRKMAGVAKPNVTISINGDVITIKSESTFKNTEISFKLGEEFDETTADDRKTKNVITLGNGSLKQVQKWDGKGTIIKRKVVDGNLVVECTMNNVTSKRVYERA

SEQ ID NO: 223 - protein, 132 aa, Bos taurus
MCDAFVGTWKLVSSENFDDYMKEVGVGFATRKVAGMAKPTLIISLNGGVVTIKSESTFKNTEISFKLGQEFDEITPDDRKVKSIVNLDEGALVQVQNWDGKSTTIKRKLMDDKMVLECVMNGVTATRVYERA

SEQ ID NO: 224 - protein, 132 aa, Bos taurus
MCDAFVGTWKLVSSENFDDYMKEVGVGFATRKVAGMAKPTLIISLNGGVVTIKSESTFKNTEISFKLGQEFDEITPDDRKVKSIVNLDEGALVQVQNWDGKSTTIKRKLVDDKMVLECVMNGVTATRVYERA

SEQ ID NO: 225 - protein, 132 aa, Cervus elaphus
MCDAFVGTWKLVSSENFDDYMKEVGVGFATRKVAGMAKPTLIISVNGDVVTIKSESTFKNTEISFKLGQEFDEVTPDDRKVKSTINLDGGALVQVQNWDGKSTTIKRKLVDDKLVLECVMNGVTATRVYERA

SEQ ID NO: 226 - protein, 132 aa, Sus scrofa
MCDAFVGTWKLVSSENFDDYMKEVGVGFATRKVAGMAKPNLIITVNGDMITIRSESTFKNTEIAFKLGQEFDEVTADDRKVKSTITLDGGALVQVQKWDGKTTTINRKIVDDKLVVECIMKGVTATRIYERA

SEQ ID NO: 227 - protein, 134 aa, Anas platyrhynchos
MAIDAFLGKWCLVSSEGFEEYMKELGVGMAMRKMGSMAKPDVYIIKDGDTITVKTESTFKTSQFSFKLGEKFEESTLDGRKTQTLINLKDDGSLIQEQEWDGKKTIITRKLVDGQLVVECDMNGVKCIRVYQKA

SEQ ID NO: 228 - protein, 107 aa, Bos taurus
MATVQQLVGRWRLVESKGFDEYMKEVGVGMALRKVGAMAKPDCIITSDGKNLSIKTESTLKTTQFSCKLGEKFEETTADGRKTQTVCNFTDGALVQHQEWDGKESTI

SEQ ID NO: 229 - protein, 135 aa, Bos taurus
MATVQQLVGRWRLVESKGFDEYMKEVGVGMALRKVGAMAKPDCIITSDGKNLSIKTESTLKTTQFSCKLGEKFEETTADGRKTQTVCNFTDGALVQHQEWDGKESTITRKLEDGKLVVVCVMNNVTCTRVYEKVE

SEQ ID NO: 230 - protein, 135 aa, Bubalus bubalis; identical to SEQ ID NO: 229

SEQ ID NO: 231 - protein, 135 aa, Cervus elaphus; identical to SEQ ID NO: 229

SEQ ID NO: 232 - protein, 134 aa, Gallus gallus
MAIDAFLGKWCLVSSEGFEDYMKELGVGMAMRKMGSMAKPDVYIIKEGDTITVKTESTFKTSQFSFKIGEKFEENTLDGRKTQTLVSLKDDGSLIQEQEWDGKKTVITRKLVDGQLVVECDMNGVKCVRVYQKA

SEQ ID NO: 233 - protein, 96 aa, Oryctolagus cuniculus
MANIQQLVGRWRLVDSQGFDEYMKELGVGLALRKMGGMANHIVSSPTTAKTSPSNREYFENNTVYLSLGEKFEETTADGRKLRSVIHVIRLQKFLE

SEQ ID NO: 234 - protein, 135 aa, Ovis aries
MATIQQLVGRWRLVESKGFDEYMKEVGVGMALRKVGAMAKPDCIITSDGKTLSIKTESTLKTTQFSCKLGEKFEETTADGRRTQTVCNFTDGALVQHQEWDGKESTITRKLEDGKLVVVCVMNNVTCTRVYEKVE

SEQ ID NO: 235 - protein, 135 aa, Sus scrofa
MASIQQLVGRWRLVESKGFDEYMKEVGVGMALRKMGAMAKPDCIITFDGKDLTIKTESTLKTTQFSCNLGQKFEETTADGRKTQTVCDFTNGELVQHQEWDGKESTITRKVENGKLVVVCVMNNVTCTRVYEKVE

SEQ ID NO: 236 - protein, 128 aa, Bos taurus
MAFTGKYETESEKNYDEFMKRLGLSSDRIEKGRNFKVISEIQQDGQNFTWSQHYPGGHSISNNFTIGKETEMETVGNKKFKVTVKMEGGKVVVDSANYHHTVEIVDGKLVEVSTFGGVIYERVSKKVA

SEQ ID NO: 237 - protein, 128 aa, Oryctolagus cuniculus
MAFTGKFEMESEKNYDEFMKLLGLPSDVVEKSRNIKIVTEIKQDGQDFTWSHHYSGGQIMTNKFTIGKESEIQTFGGKKFKAVVNMEGGKVVANFPNYQHTSEIKGDKLVEVSSIGGVTYERVSKRLA

SEQ ID NO: 238 - protein, 128 aa, Ovis aries
MAFTGKYEIESEKNYDDFMKRLGLSSDTIEKGRNFKVISEIQQDGQNFIWSQHYPGGHSISNNFTIGKETEMETVGNKKFKVTVKMEGGKVVVDSANYHHTAEIVDGKLVEVSTLGGVTYERVSKKVA

SEQ ID NO: 239 - protein, 127 aa, Salmo salar
MAFAGKWETETQEGYEEFCKLVGIPDDIIEKGRDYKLITEVVQNGNEFSWSQLYPTNKSVTNKFVIDQECDMETIGGKKFKATVTMEGGKLSTKFPKYHHTSEISGGKLIETSTVGSVVLKRTSKKI

SEQ ID NO: 240 - protein, 128 aa, Sus scrofa
MAFTGKYEIESEKNYDEFMKRLALPSDAIDKARNLKIISEVKQDGQNFTWSQQYPGGHSITNTFTIGKECDIETIGGKKFKATVQMEGGKVVVNSPNYHHTAEIVDGKLVEVSTVGGVTYERVSKKLA

SEQ ID NO: 241 - protein, 132 aa, Bos taurus
MVEAFCATWKLTESQNFDEYMKALGVGFATRQVGNVTKPTVIISQEGDRVVIRTQSTFKNTEISFHLGEEFDETTADDRNCKSVVSLDGDKLVHVQKWDGKETNFVREIKDGKMIMTLTFGDVVAVRHYEKA

SEQ ID NO: 242 - protein, 132 aa, Gallus gallus
MVEAFCATWKLADSHNFDEYMKALGVGFAMRQVGNVTKPTVIISSEGDKVVIRTQSTFKNTEISFKLGEEFDETTPDDRNCKSVVTLDGDKLVHVQKWDGKETNFVREIKDGRMVMTLTFGDVVAVRHYEKA

SEQ ID NO: 243 - protein, 132 aa, Salmo salar
MVDAFCATWKLVDSDNFDEYMKALGVGFATRQVGNVTKPTVIIAKEGDKVVVKTQSTFKNTEISFKLGEEFDEATADDRNCKSTVSLDGDKLAHVQKWDGKETKFVREIKDGKLVMTLTFEDIVAVRTYEKA

SEQ ID NO: 244 - protein, 132 aa, Salmo salar
MVDAFCATWKLVDSENFDDYMKALGVGFATRQVGNVTKPTVIISKEGDKVVVKTQSTFKNTEISFKLGEEFDEATADDRNCKSTVSLDGDKLAHVQKWDGKETKFVREIKDGKLVMTLTFEDIVAVRTYEKA

SEQ ID NO: 245 - protein, 132 aa, Bos taurus
MVEPFLGTWKLVSRENFDEYMKELGVSVAVQNLAGSAKPRIIISADGDKISIKTQSAFKNSEISFKLGEEFDETTIDNRKVKSIITLDGGSMIHVQKWLGKETTIKRKIVDGKMLVEYIMNNVVSTGVCEKL

SEQ ID NO: 246 - protein, 132 aa, Bubalus bubalis
MIEPFLGTWKLVSSENFDEYMKELGVSVAVQNLAGSAKPRIIISADRDKISIKTESSFKNSEISFKLGKEFDETTIDNRKVKSIITLDGGSMIHVQKWLGKQTTIKRKIVDGKMLVEYIMNNVVSTAVYEKV

SEQ ID NO: 247 - protein, 132 aa, Cervus elaphus
MIEPFLGTWKLVSIENFDEYMKELGVSVAVQNLAESAKPRIIISADGDKVTIRTESSFKNSEISFKLGEEFDETTIDNRKVKSIITLGGDSMIHVQKWLGKETTIKRKIVDGKMVVEYIMNNVVSTGVYEKV

SEQ ID NO: 248 - protein, 133 aa, Oryctolagus cuniculus
MMVEPFLGTWKLVSSENFEDYMKELGVSSAARNAAGLVKPSVSISVNGDTVNIRTESPFRNTEISFKLGEEFDETTADNRKVKSTVTLDSGSMIQVQKWLDKETTIKRKIVDGKMVVECTMNNIVSTRIYEKV

SEQ ID NO: 249 - protein, 132 aa, Ovis aries
MIKPFLGTWKLVSSENFDEYMKELGVSVAVQNLAGSAKPRIIISADGDKVSIKTESTFKNSEISFKIGEEFDETTIDNRKVKSIVTLDGGSMIHVQKWLGKETTIKRKIVDGKMAVEYIMNNVVSTGVYEKV

SEQ ID NO: 250 - protein, 448 aa, Ovis aries
MPGFKRILTVTILALCLPSPGNAQQQCTNGFDLDRSSGQCLDVDECRTIPEACRGDMMCVNQNGGYLCIPRTNPVYRGPYSNPYSNPYSASYPAAAPPLSAPNYPTISRPLICRFGYQMDESNQCVDVDECATDSHQCNPTQICINTEGGYTCSCTDGYWLLEGQCLDIDECRYGYCQQLCANVPGSYSCTCNPGFTLNEDGRSCQDVNECATENPCVQTCVNTYGSFICRCDPGYELEDDGVHCSDMDECSFSEFLCQHECVNQPGTYFCSCPAGYILLDDNRSCQDINECEHRNHTCILQQTCYNLQGGFKCIDPIRCEEPYLRISDNRCMCPAENPGCRDQPFTILYRDMDVVSGRSVPADIFQMQATTRYPGAYYIFQIKSGNEGREFYMRQTGPISATLVMTRPIKGPRDIQLDLEMITVNTVINFRGSSVIRLRIYVSQYPF

SEQ ID NO: 251 - protein, 296 aa, Ovis aries
MASHRHSGPSSYKVGTMAEKFNCHYCRDNLQGKKYVQKDGHHCCLKCFDKFCANTCVECRKPIGADSKEVHYKNRYWHDTCFRCSKCLQPLASETFVAKDNKILCNKCTTREDNPKCKGCLKPIVAGDQNVEYKGTVWHKDCFTCSNCKQVIGTGSFFPKGEDFYCVTCHETKFAKHCVKCNKAITSGGITYQDQPWHADCFVCVTCSKKLAGQRFTAVEDQYYCVDCYKNFVAKKCAGCKNPITGFGKGSSVVAYEGQSWHDYCFHCKKCSVNLANKRFVFHQEQVYCPDCAKKL

SEQ ID NO: 252 - protein, 279 aa, Gallus gallus
MPHQYPALTPEQKQELHDIAKRIVAPGKGILAADESTGSIAKRLSSVGAENTEENRRWYRQLLFTADNRVDPCIGGVILFHETLYQKADDGRPFPQVIKAKGGVVGIKVDKGVVPLAGTNGETTTQGLDGLMERCAQYKKDGADFAKWRCVLKISEHTPSRLAIMENANVLARYASICQQNGIVPIVEPEILPDGDHDLKRCQYVTEKVLAAVYKALSDHHIYLEGTLLKPNMVTAGHSCTKKYSPEEIAMATVTALRRTVPPAVPGGSPGPIGTAPAP

SEQ ID NO: 253 - protein, 136 aa, Ovis aries
MACQGLVASNLNLKPGECLRVRGEVAADAKSFSLNLGKDDNNLCLHFNPRFEAHGDINTIVCNSKDAGAWGAEQRESAFPFQPGSVAEVCVSFNETDLTIKLPDGYEFKFPNRLNLEAINYLAAGGDFKIKCVAFE

SEQ ID NO: 254 - protein, 291 aa, Gallus gallus
MVREQYTTTTPGTSLERPKNEYVYKIGIYGWRKRCLYLFVLLLLIILVVNFSLTIWILKVMWFSPTGMGHLRVTKEGLRLEGESEFLFPLYAKEIHSRVDSSLLLQSTHNVTVNARNSNGEVTGRLNVGPKMVEFHGQQFQINSKDGKPLFTVDENEVVIGTDKLRVTGPEGALFEHSVETPLVKAEAFKQLRLESPTRSLSMDAPRGINIKAQAGNIEALSQMDIKLHSSDGVLLLDAETVRLPKLPEGTRGGPGVSQGLYEICVCPDGKLYLSVAGLGSTCQEYSRVCQ

SEQ ID NO: 255 - protein, 778 aa, Gallus gallus
MGKQGFGYIFLTIFCTMALKLNCVSSVSVAGLGYVVTAAVVLSAVPVSMVEHAEFSKAGKEPGLQIWRIEKFDLVPVPKNLYGDFFTGDSYLVLNTIRQRSGNLQYDLHFWLGDESSQDERGAAAIFTVQMDDYLQGKAVQHREVQGHESSTFLGYFKSGIKYKAGGVASGFRHVVPNEVTVQRLLQVKGRRTVRATEVPVSWESFNTGDCFILDLGSNIYQWCGSNSNRQERLKATVLAKGIRDNEKNGRAKVFVSEEGAEREEMLQVLGPKPSLPQGASDDTKTDTANRKLAKLYKVSNGAGNMAVSLVADENPFSQAALNTEDCFILDHGTDGKIFVWKGRSANSDERKAALKTATDFIDKMGYPKHTQVQVLPESGETPLFKQFFKNWRDKDQTEGLGEAYISGHVAKIEKVPFDAATLHTSRAMAAQHGMEDDGSGKKQIWRIEGSEKVPVDPATYGQFYGGDSYIILYDYRHAGKQGQIIYTWQGAHSTQDEIATSAFLTVQLDEELGGSPVQKRVVQGKEPPHLMSMFGGKPLIVYKGGTSREGGQTTPAQTRLFQVRSSTSGATRAVELDPAASQLNSNDAFVLKTPSAAYLWVGRGSNSAELSGAQELLKVLGARPVQVSEGREPDNFWVALGGKAPYRTSPRLKDKKMDAYPPRLFACSNKSGRFTIEEVPGDLTQDDLATDDVMILDTWDQVFVWIGKDAQEEEKTEALKSAKRYIETDPASRDKRTPVTLVKQGLEPPTFSGWFLGWDDDYWSVDPLQRAMADVDV

SEQ ID NO: 256 - protein, 624 aa, Cervus elaphus
MALGLEQAEEQRLYQQTLLQDGLKDMLDHGKFLDCVVRVGEREFPCHRLVLAACSPYFRARFLAEPESAGELRLEEVSPDVVAQVLHYLYTSEIALDEASVQDLFAAAHRFQIPSIFTICVSFLQKRLCLANCLAVFRLGLLLDCARLAVSARDFICARFSLVARDADFLGLSADELIAIISSDGLNVEKEEAVFEAVMRWASSGDAKAQAERQRALPTVFESVRFRLLPRAFLESRVERHPLVRSQPELLRKVQMVKDAHEGRITVLRKKKKEKGKESAGAKATDKGTTEAKVEEEEEEAERILPGILNDTLRFGMFLQDLIFMISEEGAVAYDPAANECYCASLSTQIPKNHVSLVTKENQIFVAGGLFYNEDNKEDPMSAYFLQFDHLDSEWLGMPPLPSPRCLFGLGEALNSIYVVGGRELKDDEQSLDSVMCYDRQSFKWGESDPLPYAVYGHSVLSHMDLVYVIGGKGSNRKCLNKMSVYDPKKFEWRELAPMKTPRSLFGATIHDGRIFVAAGVTDTGLTSSAEVYNIADNKWAPFEAFPQERSSLSLVSLAGTLYAIGGFATLETESGELVPTELNDIWRYNEDEKKWEGVLREIAYAAGATFLPVRLNVLRLTKL

SEQ ID NO: 257 - protein, 566 aa, Cervus elaphus
MSEHSRNSDQEEIDGEIDEDEILANLSPEELKELQSEMEVMAPDPRLPVGMIQKDQTDKPPTGNFDHKSLVDYMYWQKASRRMLEDERVPVTFVPTKEKTQEQHEAIDRSNKNISQYLKEKLNSEIAAHKRESQGSDNVQETNDDDDEDNEDEEEDEEDDAEDEGKEDSEESDETKRGEEGEVKEQIRNGENTCQPVTSKVTEEQKGRPEAQEKTEKKISKLDPKKLALDTSFLKVSARPSGNQTDLDGSLRRVRQNDPDMKELNLNNIENIPKEMLLDFVNAMKKNKHIKTFSLANVGADENVAFALANMLRENRSITTLNIESNFITGKGIVAIMRCLQFNETLTELRFHNQRHMLGHHAEMEIARLLKANNTLLKMGYHFELPGPRMVVTNLLTRNQDRQRQKRQEEQKQQQLKEQRKLIAMLENGLGLPPGMWEMLGGPMPDSRTQEFLQPPPPRPPNPQAVPFSRQNEAMKKPSQPPKYRTDPDSFRVVKLKRIQRKSRMPEAREPPEKTNLKDVIKTLKPVPRNRPPPLVEITPRDQLLNDIRHSNIAYLKPVQLPKELA

SEQ ID NO: 258 - protein, 173 aa, Bos taurus
MRALLLTLGLVASLQAQDTLVSDSWTEDVSGKWYLKAVTTDQDVPGKNQESVTAMTFSVLEGGDLEAKVTSRVDGQCQETSLVLEQTNDPGRYTAYGGKREVLVLPLRAQDHFILYCDGELDGRQIRVARLLGRNQENSPEAWEEFTEFAKAKGLNLEIFRPLQSETCSPGGN

SEQ ID NO: 259 - protein, 176 aa, Sus scrofa
MMRALLLAIGLGLVAALQAQEFPAVGQPLQDLLGRWYLKAMTSDPEIPGKKPESVTPLILKALEGGDLEAQITFLIDGQCQDVTLVLKKTNQPFTFTAYDGKRVVYILPSKVKDHYILYCEGELDGQEVRMAKLVGRDPENNPEALEEFKEVARAKGLNPDIVRPQQSETCSPGGN

SEQ ID NO: 260 - protein, 195 aa, Gallus gallus
MQPLPQHPDPKARMTAVLPSLVLTLLCLLRVGAEVPVQLDFDTKKFAGMWHMMAAVSNCPVFLGMKDKMTSSATAVSFTPEGHMTMETVIPLPEECKKIMMLFQQSGQAGHYIGTEKGEKRDLRVMDTDYEHYAIVYSQQKDSQEPSTMLQLYTRNQDMSPQLLQKFKELFHSMGLTEDMLVVLPHSDRCAKTQK

SEQ ID NO: 261 - protein, 178 aa, Gallus gallus
MRTLALSLALALLCLLHTEAAATVPDRSEVAGKWYIVALASNTDFFLREKGKMKMVMARISFLGEDELEVSYAAPSPKGCRKWETTFKKTSDDGEVYYSEEAEKTVEVLDTDYKSYAVIFATRVKDGRTLHMMRLYSRSREVSPTAMAIFRKLARERNYTDEMVAVLPSQEECSVDEV

SEQ ID NO: 262 - protein, 182 aa, Salmo salar
MFLVVTVFSVMLWTQCINADIQPQQDFDLQRFSGQWYRVGLAYDSPEFVPWRSYVQVSNGNITSDMDGNANLTVWGTGLKGCMCRVYYYQKTDLPGQFNYFSDRHQILKDITVVESNYTEYGLVVKYKKIVKEFSQVSLYGRSPTLRKELVERFRNYSLSLGFSADSILTPLFVDPCSDCGH

SEQ ID NO: 263 - protein, 1398 aa, Salmo salar
MATKVMHFNQKKHVSHVSHHSSHTTKHVVKEQSSRKSSSKSVNQVQHTHATEAMAEPAYTVPAFRQRSADEIEEYQRVSSHVGKGLASIQKELHRMRVVTKTQVENIAIKREVQEMMHKKMTLSTETDKAPDFMVALRPHTVWEKTPVKLFCTVDGHPRPIVKWYKGGKPVDPLSAPGKYKIESKYGVHSLIISRCMVSDTAEYSAVATNSHGSTTSKASVIVKRPAGGAYGSCQLFGQVPHLTVIHHSKLEITMLDRFGVSFGTEGGSISLVCTMVVVPDLPNVPPLAQWYRDDKLLKAGKLAEIKVGGGAATLTLPHLAKDDEGLYTLRMWTKDGTTEHSAYLFVKDAAPSVAGAPGAPISVKAFDINSDYVLVAWKPPNTTNEAAITGYFVDKRESGSATWSQCNDAPVQICKYPVHGLNVGHAYHFRVRAVNSAGISRPSRESDKVTALDPAERERLQVIKLDGKHEVVIKDDDLEGVPSAPGQVVATRNTKSSVFVQWDPPKHPNHLMGYYIDASLVGSKTWAPCNHKPYKNTRFVVHGLTPGETYVFRVQAVNVYGLSDESQESTPIAVEPALTTPSAPHGITMLSCDGASMIIAWNSPKHRGGSKINAYYVDKRDADSLAWKEVNSAPTNTRTYTVDGLTEGTFYEFKVQAGNLAGVGVPSAPSTPLKCEAWTSAVPGPAYDLAFREVRGDSLVILWKAPVYTGASAVTGYIVEMAKKGHHYHPLNEEAIGHCYLQVTGLEAGAEYTFKVKAVNAEGVGKASQTSEPVFAKALPGTQEIQCGVDEDTGDIFLSFEACEISETSNFAWSKNYKEISDCTRVSIETKGKHSKLTFVNPDVSDLGAFSVHVTDTDGVSASHTVTEDALNTMLELSYNIRHPIVPLKHQLNYEVLEKGHVRFWLQCLKMSPAVNYRFIVNDKEVTSSDSHKISHDVNTGVIQMTVDHFSKASEATYTVQIHDGRAKNQSSLVLVGDVFKAALKEAEFQRSEHIRKQGPHFAEYLSYHVDDDCTVLLVCKVANVKKETVFHWFKDEEEIVPETPPNVMSGSVALPISLFSRKDQGHYKAKLSDDRGKDTSVFDISGQVFDDIINAIAHIAGSSASELVLQCTPEGIRLQCYMKYYTEEIRTAWLHKDSKISSSEKMRIAGTAEMAWMQIREPSEKEKGHYSIQIMDATKSHTRTFDLSGQAYTDAYEEYLRLKAAAFAEKNRGRVVGGLPDVVTIMEQKTLSLTCTVWGDPSPEVTWFKNENEVVSTEHAKITLEANKFASLTITAVTSEDSGKYSINVRNKYGGEFVEITVSVYKQGESPPEPKMGGRGATPAPLASKTPAKTPTPAQTPTPSLKSPTPSLKSPTPSLRSPAKSPTPTPKSPTPPRRVKSPSPARSLKSPTPPRK

SEQ ID NO: 264 - protein, 150 aa, Gallus gallus
MSFSPDEINDFKEAFLLFDRTGDAKITLSQVGDIVRALGQNPTNAEINKILGNPSKEEMNAKKITFEEFLPMLQAAANNKDQGTFEDFVEGLRVFDKEGNGTVMGAELRHVLATLGEKMTEEEVEELMKGQEDSNGCINYEAFVKHIMSV

SEQ ID NO: 265 - protein, 192 aa, Gallus gallus
MAPKKDVKKPAAAAAPAPAPAPAPAPAPAKPKEPAIDLKSIKIEFSKEQQDDFKEAFLLFDRTGDAKITLSQVGDIVRALGQNPTNAEINKILGNPSKEEMNAKKITFEEFLPMLQAAANNKDQGTFEDFVEGLRVFDKEGNGTVMGAELRHVLATLGEKMTEEEVEELMKGQEDSNGCINYEAFVKHIMSV

SEQ ID NO: 266 - protein, 170 aa, Ovis aries
MAPKKAKRRAAAEGGSSNVFSMFDQTQIQEFKEAFTVIDQNRDGIIDKEDLRDTFAAMGRLNVKNEELDAMMKEASGPINFTVFLTMFGEKLKGADPEDVITGAFKVLDPEGKGTIKKQFLEELLTTQCDRFSQEEIRNMWAAFPPDVGGNVYYKNICYVITHGDAKDQE

SEQ ID NO: 267 - protein, 232 aa, Cervus elaphus
MRCPGVIKAGCRRLTYPALGFLLSTPASVLTASMAPKKPDPKKDEAKAGAKAAAAPAPAPAPAPAPEPSKEPEFDPSKVKIEFTPEQIEEFKEAFMLFDRTPKCEMKITYGQCGDVLRALGQNPTQAEVLRVLGKPKQDELNSKMMDFDTFLPMLQHISKNKDTGTYEDFVEGLRVFDKEGNGTVMGAELRHVLATLGERLTEDEVEKLMAGQEDSNGCINYEAFVKHIMAS

SEQ ID NO: 268 - protein, 699 aa, Gallus gallus
MDKIEDMAMMTHLHEPAVLYNLKERYAAWMIYTYSGLFCVTVNPYKWLPVYNPEVVLAYRGKKRQEAPPHIFSISDNAYQFMLTDRENQSILITGESGAGKTVNTKRVIQYFATIAASGEKKKEEQSGKMQGTLEDQIISANPLLEAFGNAKTVRNDNSSRFGKFIRIHFGATGKLASADIETYLLEKSRVTFQLKAERSYHIFYQVTSNKKPELIEMLLITTNPYDYPFVSQGEITVPSIDDKEELMATDSAIDILGFTADEKTAIYKLTGAVMHYGNLKFKQKQREEQAEPDGTEVADKAAYLMGLNSADMLKALCYPRVKVGNEYVTKGQTVQQVHNAVGALAKAVYERMFLWMVVRINQQLDTKQPRQYFIGVLDIAGFEIFDFNSFEQLCINFTNEKLQQFFNHHMFVLEQEEYKKEGIEWTFIDFGMDLAACIELIEKPMGIFSILEEECMFPKATDTSFKNKLYDQHLGKSSNFQKPKPTKGKVEAHFSLIHYAGTVDYNITGWLEKNKDPLNETVIGLYQKSSLKTLALLFANYGGAEAEASAGAGKKGGKKKGSSFQTVSALFRENLNKLMTNLRSTHPHFVRCIIPNETKTPGAMEHELVLHQLRCNGVLEGIRICRKGFPNRVLYADFKQRYKVLNASAIPEGQFIDSKKACEKLLGSIDIDHTQYKFGHTKVFFKAGLVGLLEEMRD

SEQ ID NO: 269 - protein, 499 aa, Ovis aries
MFNYERPKHFIQSQNPCGSRLQPPGPEISSYSSQTKQSSITIQPRQCTEQRYSASSTVSSHITVSSSAFPASPQQLAGSNPSQRVTATYNQSPASFLSSILPSQPDYSSSKIPSTVDSNYQQPSVGQPVNVKSSQNANAKLTPRTPDHEIQGSKEALIQDLERKLKCKDSLLHNGNQRLTYEEKMARRLLGPQNAAAVFQGQNDSEAQDSAQQHNIEHARLQVPTSQVRSRSSSRGDMSDQDAIQEKFYPPRFIQVPENMSIEEGRFCRMDFKVSGLPAPDVSWYLNGRPVQSDDFHKMIVSEKGFHSLIFEVVRASDAGAYACVAKNRAGEATFTVQLDVLAKEHRRAPMFIYKPQSKKVFEGESVKLECQISAIPPPKLFWKRNNEMVQFNTDRISLYHDNSGRVTLLIKDVNKKDAGWYTVSAVNEAGVTTCNTRLDVTARPNQTLPAPKQLRVRPTFSKYLALNGKGLNVKQAFNPEGEFQRLAAQSGLYESEEL

SEQ ID NO: 270 - protein, 293 aa, Meleagris gallopavo
MPLAGTPAPLKRKKPTKLIGKLTHEVMPQEVTKLNLGKKISIPRDVMLEELSLLTNKGSKMFKLRQLRVEKFIYENNPDAFSDNSVDHFQRFIPSGGHYGEDAHGYGHGRMVGGVTAGQHGSSKQHYSTVPPRPGSKGGPGNSEGEHAEKSAGSAGEGGHGTEKDGKSGGKKPLLKTYISPWERAMGISPEDKSQLTIDLLSYSPKADFPHYKSFNRTAMPYGGYEKAAKRMTFKVPQFDICPLLPESIVLYNQNFRNRPSFNRTPIPWMPSGESSEYHTDINVPRSGETEEL

SEQ ID NO: 271 - protein, 244 aa, Ovis aries
MIPKEQKGPVMTTTGDFTEPVPLLDLGKKLSVPQDLMMEELSLRNNRGSLLFQKRQRRVQKFTFEFAASPRAVVAGSAKEKVTGTAEPGMVANSHEGQNYRSELHIYPASHGDPGDAESMASRPQRHNPSALAPGYAEPLKGIPPEKFNHTAIPKGYRCPWQEFISYRDYLGEGGSHTPSLVEYRNFNKTPVPFGGTLVGETLPRAGTPFVPELVSGLQLLRLTPSFNRVAQGWVRNLPESEEL

SEQ ID NO: 272 - protein, 302 aa, Cervus elaphus
MPLSGTPAPNKKRKSSKLIMELTGGGQESSGLNLGKKISVPRDVMLEELSLLTNRGSKMFKLRQLRVEKFIYENHPDVFSDSSMDHFQKFIPTVGGQLGMAGQGFSYSKSSAGGQAGRSGSAGQFGSDQQHHRQGSGSGSGPGSGGAGGPGGHAGRGGAAGTAGAGETGTDNQAGGDGKHVTVFKTYISPWEKAMGVDPQQKVELGIDLLAYGAKAELPKYKSFNRTAMPYGGYEKASKRMTFQMPKFDLGPLLSEPLVLYNQNLSNRPSFNRTPIPWLSSGEPVDYNVDISIPLDGETEEL

SEQ ID NO: 273 - protein, 337 aa, Ovis aries
MWTCLPDQSDVIHARRAYDLQSDNLYKSDLQWLKGIGWLPSGSLEDEKNKRATQILSDHVYRQHPDKFKFSSLMDSIPMVLAKNNAITMNHRLYTEAWDKDKTTVHIMPDTPGVLLAKQNQINYSEKLYKLGLDEAKRKGYDMRIDAIPIRAAKASRDIASEFKYKEGYRKQLGHHIGARAIHDDPKMMWSMHVAKIQSDREYKKDFEKWKTKFSSPVDMLGVVLAKKCQTLVSDIDYKNYLHQWTCLPDQSDVIHARQAYDLQSDNVYKSDLQWLKGIGWVPIGSLDVEKCKRASEILSDKLYRQPPDKFKFTSVTDSLEQVLAKNNAINMNKRLY

SEQ ID NO: 274 - protein, 367 aa, Gallus gallus
MDHEKQKGHYVGVPNAKADTKIRFALGIGKVQSELEYKKHFAKWKTQCHLPVDMLSIQSAKHGQSLVSDVDYRHYLHQWICLPDQNDVIHARKAYDLQSDAVYKSDLEWLRGIGWLPNDSLGINHVKHAGDLLNERKYRTKAETLHFTPVADRVDYVTAKKSGEILSDIKYHKDWNEVKSNYTLTDTPQLDMAREAARILNQSLYKESWEKEKATGYLLPPDTVQIRHAKHSNDVQSELKYKADYVKQRGHYVGVASMRDDPKLVWFEHAGEIQNDRLYKSNYHKTKSKIHIPADIMSVVAAKECQALVSDVDYRHYLHQWTCHPDQNDCIQARKAYDLQSDNIYKSDLEWLRGCGWIPLGSVEHKK

SEQ ID NO: 275 - protein, 186 aa, Bos taurus
MASKRALVILAKGAEEMETVIPVDVMRRAGIKVTVAGLAGKDPVQCSRDVVICPDASLEDAKKEGPYDVVVLPGGNLGAQNLSESAAVKEILKEQEKRKGLIAAICAGPTALLAHEIGFGSKVTTHPLAKDKMMNGSHYSYSENRVEKDGLILTSRGPGTSFEFALKIVEVLVGKEVADQVKAPGS

SEQ ID NO: 276 - protein, 562 aa, Gallus gallus
MVHIETVKTKAYADQKPGTSGLRKRVTVFQNNAHYAENFIQSILATVPPAERQEATLVVGGDGRFYMKDAIQIIVRIAAANGIGRLVIGQNGILSTPAVSCIIRKIKAIGGIILTASHNPGGPNGDFGIKFNTANGGPAPEGITDKIFQISKKIEEYAICPDLKVDLGTIGKQQFDLENKFKPFTVEIVDSVEAYANMLRNIFDFNALKELLSGKNHLKIRIDAMHGVVGPYVKKILCEELGAPANSAVNCTPLEDFGGHHPDPNLTYAADLVQTMKTGEYDFGAAFDGDGDRNMILGKHGFFVNPSDSVAVIAANIFSIPYFQQTGVRGFARSMPTSGALDRVAHATKIALYETPTGWKFFGNLMDANKLSLCGEESFGTGSDHIREKDGLWAVLAWLSILAARKQSVEDIMKDHWQKYGRNFFTRYDYEEVDADAAGKMMKDLETVMFDRSFVGKQLSAGDKVYTVEKADNFEYNDPVDGSVSRNQGLRLIFSDGSRIIFRLSGTGSAGATVRLYIDSYEKDAKKIHEDPQVMLAPLISIALKLSQLHERTGRTGPTVIT

SEQ ID NO: 277 - protein, 525 aa, Gallus gallus
MPLSADPHFKKLLEWHKANASKLVLRQLFEADKDRFHKFSLTLNTDHGDILLDYSKNLVTEEVMKMLIELAKSRGVESARERMFSGEKINFTENRAVLHIALRNRSNTPILVDGKDVVPEVNKVLDKMKHFCQGPLMVTEALKPYSKGGPRVWFVSNIDGTHIAKTLAELHPETTLFIIASKTFTTQETITNAVTAKEWFLHAAKDPSAVAKHFVALSTNGPKVKEFGIDTENMFEFWDWVGGRYSLWSAIGLSIALHIGFDNFESLLAGGHWMDKHFHTAPLEKNVPVLLAMLGVWYINCYGCETHALLPYDQYMHRFAAYFQQGDMESNGKYITKKGSRVDYNTGPIVWGEPGTNGQHAFYQLIHQGTRMIPCDFMIPVQTQHPVRNGLHHKILLANFLAQTEALMKGKTADEARKELQAAGLSGDALEKLLPHKVFEGNRPTNSIMFTKLNPFTLGAIIAMYEHKIFVQGVVWDINSYDQWGVELGKQLAKKIEPELESDAPVTSHDSSTNGLISFIKKHRA

SEQ ID NO: 278 - protein, 417 aa, Gallus gallus
MSLSNKLTLDKVDVKGKRVVMRVDFNVPMKDHKITNNQRIKAAVPTIKHCLDHGAKSVVLMSHLGRPDGVPMPDKFSFSPVAVELKALLGREVSFLKDCVGPEVEKACANPANGSVILLENLRFHVEEEGKGKDASGNKIKADAAKVEAFRASLSKLGDVYVNDAFGTAHRAHSSMVGVHLPQKAAGFLMKKELDYFAKALESPERPFLAILGGAKVQDKIQLISNMLDKVNEMIIGGGMAFTFLKVLNNMQIGNSLFDEEGSKIVKDLMAKAEKNGVKITLPVDFITADKFDEHAQTGEATVASGIPAGWMGLDCGPESVKKFVEVVGRAKQIVWNGPVGVFEWDKFSKGTKALMDKVVEVTGKGCITIIGGGDTATCCAKWNTEDKVSHVSTGGGASLELLEGKVLPGVDALSSV

SEQ ID NO: 279 - protein, 188 aa, Ovis aries
MNQKKRIGYRKALLKGQGGGEEQEVAAQLAEPRSGRQRVPQALRLPPPASPSPARGPSSSPESSPNSSAMAGWNAYIDNLMADGTCQDAAIVGIDSPSVWAAVPGKTFVNITPAEVCILVGKDRSSFFVNGLTLGGQKCSVIRDSVLQDGEFTMDFRTKSTGGAPTFNITVTMTAKTLVLLMGKEMKQ

SEQ ID NO: 280 - protein, 140 aa, Ovis aries
MAGWNAYIDNLMADGTCQDAAIVGYKDSPSVWAAVPGKTFVNITPAEVGILVGKDRSSFFVNGLTLGGQKCSVIRDSLLQDGEFTMDLRTKSTGGAPTFNITVTMTAKTLVLLMGKEGVHGGMINKKCYEMASHLRRSQY

SEQ ID NO: 281 - protein, 140 aa, Gallus gallus
MAGWQSYVDNLMCDGCCQEAAIVGYCDAKYVWAATAGGIFQSITPVEIDMIVGKDREGFFTNGLTLGAKKCSVIRDSLYVDGDCTMDIRTKSQGGEPTYNVAVGRAGRVLVFVMGKEGVHGGGLNKKAYSMAKYLRDSGF

SEQ ID NO: 282 - protein, 530 aa, Gallus gallus
MSKHHDAGTAFIQTQQLHAAMADTFLEHMCRLDIDSEPTIARNTGIICTIGPASRSVDKLKEMIKSGMNVARLNFSHGTHEYHEGTIKNVREATESFASDPITYRPVAIALDTKGPEIRTGLIKGSGTAEVELKKGAALKVTLDNAFMENCDENVLWVDYKNLIKVIDVGSKIYVDDGLISLLVKEKGKDFVMTEVENGGMLGSKKGVNLPGAAVDLPAVSEKDIQDLKFGVEQNVDMVFASFIRKAADVHAVRKVLGEKGKHIKIISKIENHEGVRRFDEIMEASDGIMVARGDLGIEIPAEKVFLAQKMMIGRCNRAGKPIICATQMLESMIKKPRPTRAEGSDVANAVLDGADCIMLSGETAKGDYPLEAVRMQHAIAREAEAAMFHRQQFEEILRHSVHHREPADAMAAGAVEASFKCLAAALIVMTESGRSAHLVSRYRPRAPIIAVTRNDQTARQAHLYRGVFPVLCKQPAHDAWAEDVDLRVNLGMNVGKARGFFKTGDLVIVLTGWRPGSGYTNTMRVVPVP

SEQ ID NO: 283 - protein, 583 aa, Gallus gallus
MPKPINVRVTTMDAELEFAIQPNTTGKQLFDQVVKTVGLREVWFFGLQYVDSKGYSTWLKLNKKVTQQDVRKENPLQFKFRAKFFPEDVSEELIQEITQRLFFLQVKEAILNDEIYCPPETAVLLASYAVQSKYGDYNKEIHKLGYLANDRLLPQRVLEQHKLTKEQWEERIQNWHEEHRGMLREDSMMEYLKIAQDLEMYGVNYFEIKNKKGTELWLGVDALGLNIYEHDDKLTPKIGFPWSEIRNISFNDKKFVIKPIDKKAPDFVFYAPRLRINKRILALCMGNHELYMRRRKPDTIEVQQMKAQAREEKHQKQLERAQLENEKKKREIAEKEKERIEREKEELMERLRQIEEQTMKAQKELEEQTRRALELDQERKRAKEEAERLEKERRAAEEAKAALAKQAADQMKNQEQLAAELAEFTAKIALLEEAKKKKEEEASEWQHKAFAAQEDLEKTKEELKSVMSAPPPPPPPPVIPPTENEHDEHDENNAEASAELSSDGVMNHRSEEERVTETQKNERVKKQLQALSSELAQARDETKKTQNDVLHAENVKAGRDKYKTLRQIRQGNTKQRIDEFEAM

SEQ ID NO: 284 - protein, 135 aa, Anas platyrhynchos
MPADFNGYWKMISNDNFEEYLKALDVNVAVRKIANLLKPDKEILQNGDHMIIKTLSTFRNYIMEFDVGKEFEEDLAGVDDRKCMTTVSWEGDKLLCVQKGEKEDRGWTQWIEGDEMHLEIRVCGVKCKQVFKKVQ

SEQ ID NO: 285 - protein, 135 aa, Bos taurus
MPVDFTGYWKMLANENFEEYLRVLDVNVALRKIANLLKPDKEIVQEGDHMIIRMLSTFRNYIVDFQVGKEFEEDLTGIDDRKCMTTVSWDGDKLECVQKGEKEGHGWTQWIEGEELHLEMRMEGVVCKQVFKKVN

SEQ ID NO: 286 - protein, 135 aa, Gallus gallus
MPADFNGYWKMISNDNFEEYLKALDVNVAVRKIANLLKPDKEILQNGDHMIIKTLSTFRNYIMEFDVGKEFEEDLAGVDDRKCMTTVSWDGDKLLCVQKGEKEDRGWTQWIEGDEMHLEIRVCGVKCKQVFKKVQ

SEQ ID NO: 287 - protein, 135 aa, Meleagris gallopavo
MPADFNGYWKMISNENFEEYLKALDVNVAVRKIANLLKPDKEIFQNGDHMIIKTLSTFRNYIMEFDVGKEFEEDLAGVDDRKCMTTVSWDGDKLLCVQKGEKEDRGWTQWIEGDEMHLEIRVCGVKCKQVFKKVQ

SEQ ID NO: 288 - protein, 135 aa, Oryctolagus cuniculus
MPVDFTGYWKMLANENFEEYLRALDVNVALRKIANLLKPDKEILQDGDHMIIRTLSTFRNYIMDFHVGKEFEEDLTGIDDRKCMTTVSWDGDKLECVQKGEKEGRGWTQWIEGDELHLEMRVQGVVCKQVFKKVQ

SEQ ID NO: 289 - protein, 133 aa, Salmo salar
MPVDLNGYWKIISNDNFEEYLKALDVNVAIRKIATLLKPDKDISHDGDHVVIKTLSTFKNYNMDFHVGKQISPKKICPYDKCLTTISWEGDKLVCVQKGEKEGRGWTHWVEGDVLHLELRACGAVCKQVFQKT

SEQ ID NO: 290 - protein, 135 aa, Anas platyrhynchos
MPADYNGTWEMESNENFEGYMVALDIDFATRKVAKHLKQTKEIIQDGDNFKTKTLSTLRNYELNYTVGVEFEEHTKGLDNRVVKTLVTWDGDKLVCVQKGDKKNRGWKHWIEGDQLHLELTCEDQVCHQIFKRKK

SEQ ID NO: 291 - protein, 134 aa, Bos taurus
MTRDQNGTWEMESNDNFEGYMKALDIDFATRKIALHLTQTKIIEQDGDKFKTQTKSTFRNYNVDFTVGVEFEEHTKGLDDRKVKTLVVWEGDALVCVQKGEKENRGWKQWVEGDKLYLELTCGDQVCRQVFKKK

SEQ ID NO: 292 - protein, 134 aa, Bubalus bubalis; identical to SEQ ID NO: 291

SEQ ID NO: 293 - protein, 134 aa, Cervus elaphus
MTRDQNGTWEMESNDNFEGYMKALDIDFATRKIAIHLTQTKIIEQDGDKFKTQTKSTFRNYNVDFTVGVEFEEHTKGLDNRKVQTLVVWEGDALVCVQKGEKENRGWKQWVEGDKLYLELTCGDQVCRQVFKKK

SEQ ID NO: 294 - protein, 135 aa, Gallus gallus
MPADYNGTWEMESNENFEGYMVALDIDFATRKVAKHLKQTKEIIQDGDNFKTKTLSTLRNYELNFTVGVEFEEHTKGLDNRVVKTLVTWDGDKLVCVQKGEKKNRGWKHWIEGDQLYLELTCEDQVCHQIFKKKK

SEQ ID NO: 295 - protein, 135 aa, Meleagris gallopavo
MPADYNGTWEMESNENFEGYMVALDIDFATRKIAKHLKQTKEIIQDGDNFKTKTLSTLRNYDLDYTVGVEFEEHTKGLDNRVVKTLVTWDGDKLVCVQKGEKKNRGWKHWIEGDQLHLELTCEDQVCHQIFKKKK

SEQ ID NO: 296 - protein, 134 aa, Oryctolagus cuniculus
MTRDQNGTWEMESNDNFEGYMKALDIDFATRKIAVHLTQTKIIDQAGDSFKTKTNSTFRNYNLDFTVGVEFDEYTKGLDNRHVKTLVTWEGDCLVCVQKGEKENRGWRQWIEDGKLHLELTCGDQVCHQVFKKK

SEQ ID NO: 297 - protein, 134 aa, Ovis aries
MTRDQNGTWEMESNDNFEGYMKALDIDFATRKIAIHLTQTKIIEQDGDKFKTQTKSTFRNYNVDFTVGVEFEEHTKGLDDRKVKTLVVWEGDALVCVQKGEKENRGWKQWVEGDKLYLELTCGDQVCRQVFKKK

SEQ ID NO: 298 - protein, 135 aa, Salmo salar
MPADFNGKWELESSENFEDYMKALNIDFATRKIAVQLAQTKVIIQDGDKFEAKTLSTFRNYEQMFTVGVEYVEHTKGLDNRVVKALVVWEGDKLVCTQKGEKDNRGWKHWIEGDKLYLELTCEDKVCRQVFKRKE

SEQ ID NO: 299 - protein, 1236 aa, Anas platyrhynchos
MFRTHFFLFYSLIMCSVSAEEIFQPTLVLDMAKVLLDNYCYPENLVGMQEAIEQAIKSGEILDISDPKLLASVLTAGVQGALNDPRLVISYEPSPHAAPEKETEASPTREQLLSLIEHVIVYDKLEGNVGYLRIDYIIGQDVVQKVGAFLVDKVWKTLINTSALVIDLRHSTGGQISGLPFIISYLHEADKMLHVETVYYRPSNTTTEIWTLPKVLGERYSKEKDVIVLISRHTTGVAEDVAYILKHMNRAITVGEKTAGGSLDIQKLRIGLSNFYLMVPVSRSVSPLSGVGQSWEVNGVMPCVATEAEQALQKSLDILAVRRAVPGTLTRLIDILKDYYSLVERVPVLLRHLTTSDFSSVQSAEDLATKLNTEMQTLSEDPRLLVRVMMPGETAVPPAETPIAVAANLPDNEQLLHALVDTVFKVSVLPGNVGYMRFDEFADASVLAKLGPYIVKKVWEPLQNTENLIVDLRYNPGGPSSSAVPVLLSYFQDPAVGPVHLFTTYDRRTNHTQEHNSQAELLAQPYGAQRGIYLLTSHHTATAAEEFAYLMQSLGRATLIGEITAGSLSHTSTFPLLQPEQGITRGLTITVPVITFIDNHGESWMGGGVVPDAIVLAEDALEKAEEVLAFHRKMGMLLESTGQLLEAHYAIPEVAGKASAMLSTKRAQGGYRSAVDFETLASQLTSDLQEASGDHRLHVFHSHVEPTPEEQLPNMIPSPEELSYIIEALFKIEVLPGNLGYLRFDMMAEAETVKAIGPQLVQMVWNKLVNTDAMIIDMRYNTGGYSTAVPILCSYFFEPEPRQHLYTVFDRSTSRSTEVWTLPQVTGKRYGSLKDVYILTSHMSGSAAEAFTRSMKDLHRATVIGEPTVGGSLSVGIYRVGNSSLYASIPSQVVLSPVTGKVWSVSGVEPHITIQASEAMAVAQHIANLRAQVPQTLQTVGKLVAENYAFVDIGAVIASNLTKNINKGNYKRINSEEELARKVTANLQALSDDEHLKMLYIPEHAKDNIPGIMPKQIPSPEVFEDLIKFSFHTNVFENNIGHLRFDMFGDYELLTQVSDLLVEHVWKKIVHTDALIIDMRYNIGGDTTSIPILCSYFFDEGQHILLDKVYDRPSDSVKEIWTQPQLKGERYGSQKGLVILTSAVTSGAAEEFVSIMKRLGRALIIGEQTSGGSHSPQTYQVDDTNFYIIIPTSRSVISAESTSWEGKGVPPHMETPAATALIKAKEMLSAHLHSSR

SEQ ID NO: 300 - protein, 1286 aa, Bos taurus
MVRKWALLLPMLLCGLTGPAHLFQPSLVLEMAQVLLDNYCFPENLMGMQGAIEQAIKSQEILSISDPQTLAHVLTAGVQSSLNDPRLVISYEPSTLEAPPRAPAVTNLTLEEIIAGLQDGLRHEILEGNVGYLRVDDIPGQEVMSKLRSFLVANVWRKLVNTSALVLDLRHCTGGHVSGIPYVISYLHPGSTVSHVDTVYDRPSNTTTEIWTLPEALGEKYSADKDVVVLTSSRTGGVAEDIAYILKQMRRAIVVGERTVGGALNLQKLRVGQSDFFLTVPVSRSLGPLGEGSQTWEGSGVLPCVGTPAEQALEKALAVLMLRRALPGVIQRLQEALREYYTLVDRVPALLSHLAAMDLSSVVSEDDLVTKLNAGLQAVSEDPRLQVQVVRPKEASSGPEEEAEEPPEAVPEVPEDEAVRRALVDSVFQVSVLPGNVGYLRFDSFADASVLEVLGPYILHQVWEPLQDTEHLIMDLRQNPGGPSSAVPLLLSYFQSPDASPVRLFSTYDRRTNVTREHFSQTELLGRPYGTQRGVYLLTSHRTATAAEELAFLMQSLGWATLVGEITAGSLLHTHTVSLLETPEGGLALTVPVLTFIDNHGECWLGGGVVPDAIVLAEEALDRAQELLEFHRSLGELVEGTGRLLEAHYARPEVVGQMGALLRAKLAQGAYRTAVDLESLASQLTADLQEMSGDHRLLVFHSPGEMVAEEAPPPPPVVPSPEELSYLIEALFKTEVLPGQLGYLRFDAMAELETVKAVGPQLVQLVWQKLVDTAALVVDLRYNPGSYSTAVPLLCSYFFEAEPRRHLYSVFDRATSRVTEVWTLPHVTGQRYGSHKDLYVLVSHTSGSAAEAFAHTMQDLQRATIIGEPTAGGALSVGIYQVGSSALYASMPTQMAMSASTGEAWDLAGVEPDITVPMSVALSTARDIVTLRAKVPTVLQTAGKLVADNYASPELGVKMAAELSGLQSRYARVTSEAALAELLQADLQVLSGDPHLKTAHIPEDAKDRIPGIVPMQIPSPEVFEDLIKFSFHTNVLEGNVGYLRFDMFGDCELLTQVSELLVEHVWKKIVHTDALIVDMRFNIGGPTSSISALCSYFFDEGPPILLDKIYNRPNDSVSELWTLSQLEGERYGSKKSMVILTSTLTAGAAEEFTYIMKRLGRALVIGEVTSGGCQPPQTYHVDDTDLYLTIPTARSVGAADGSSWEGVGVVPDVAVPAEAALTRAQEMLQHTPLRARRSPRLHGRRKGHHRQSQGRAGSLGRNQGVGRPEVLTEAPSGQKRGLLQCG

SEQ ID NO: 301 - protein, 1286 aa, Bos taurus
MVRKWALLLPMLLCGLTGPAHLFQPSLVLEMAQVLLDNYCFPENLMGMQGAIEQAIKSQEILSISDPQTLAHVLTAGVQSSLNDPRLVISYEPSTLEAPPRAPAVTNLTLEEIIAGLQDGLRHEILEGNVGYLRVDDIPGQEVMSKLRSFLVANVWRKLVNTSALVLDLRHCTGGHVSGIPYVISYLHPGSTVSHVDTVYDRPSNTTTEIWTLPEALGEKYSADKDVVVLTSSRTGGVAEDIAYILKQMRRAIVVGERTVGGALNLQKLRVGQSDFFLTVPVSRSLGPLGEGSQTWEGSGVLPCVGTPAEQALEKALAVLMLRRALPGVIQRLQEALREYYTLVDRVPALLSHLAAMDLSSVVSEDDLVTKLNAGLQAVSEDPRLQVQVVRPKEASSGPEEEAEEPPEAVPEVPEDEAVRRALVDSVFQVSVLPGNVGYLRFDSFADASVLEVLGPYILHQVWEPLQDTEHLIMDLRQNPGGPSSAVPLLLSYFQSPDASPVRLFSTYDRRTNITREHFSQTELLGRPYGTQRGVYLLTSHRTATAAEELAFLMQSLGWATLVGEITAGSLLHTHTVSLLETPEGGLALTVPVLTFIDNHGECWLGGGVVPDAIVLAEEALDRAQEVLEFHRSLGELVEGTGRLLEAHYARPEVVGQMGALLRAKLAQGAYRTAVDLESLASQLTADLQEMSGDHRLLVFHSPGEMVAEEAPPPPPVVPSPEELSYLIEALFKTEVLPGQLGYLRFDAMAELETVKAVGPQLVQLVWQKLVDTAALVVDLRYNPGSYSTAVPLLCSYFFEAEPRRHLYSVFDRATSRVTEVWTLPHVTGQRYGSHKDLYVLVSHTSGSAAEAFAHTMQDLQRATIIGEPTAGGALSVGIYQVGSSALYASMPTQMAMSASTGEAWDLAGVEPDITVPMSVALSTARDIVTLRAKVPTVLQTAGKLVADNYASPELGVKMAAELSGLQSRYARVTSEAALAELLQADLQVLSGDPHLKTAHIPEDAKDRIPGIVPMQIPSPEVFEDLIKFSFHTNVLEGNVGYLRFDMFGDCELLTQVSELLVEHVWKKIVHTDALIVDMRFNIGGPTSSISALCSYFFDEGPPILLDKIYNRPNNSVSELWTLSQLEGERYGSKKSMVILTSTLTAGAAEEFTYIMKRLGRALVIGEVTSGGCQPPQTYHVDDTDLYLTIPTARSVGAADGSSWEGVGVVPDVAVPAEAALTRAQEMLQHTPLRARRSPRLHGRRKGHHRQSQGRAGSLGRNQGVVRPEVLTEAPSGQKRGLLQCG

SEQ ID NO: 302 - protein, 1286 aa, Cervus elaphus
MVRKWALLLPMLLCSLTGPAHLFQPSLVLDMAQVLLDNYCFPENLMGMQEAIEQAIKSHEILSISDPQTLAHVLTAGVQSSLNDPRLVISYEPSTLEVPPRAPAFMNLTLEEIIAGLQDGLRHEVLEGNVGYLRVDDIPGQEVMSKLRSFLVANVWRKLMNTSALVLDLRHCTRGHISGIPYVISYLHPGNTILHVDTIYDRPSNTTTEIWTLPEALGETYSADKDVVVLTSSRTGGVAEDIAYILKQMRRAIVVGERTVGGALDLQKLRIGQSDFFLTVPVSRSLGPLGEGSQTWEGSGVLPCVGTPAEQALEKALAILMLRRALPGVIQRLQEALREYYTLVDRVPALLHHLANMDLSSVVSEEDLVTKLNAGLQAVSEDPRLQVQVVRSKEASSGPEEEAEEPPETVPEVPEDEAVRRALVDSVFQVSVLPGNVGYLRFDSFADASVLEVLGPYILHQVWEPLQDTEHLIMDLRQNPGGPSSSVPLLLSYFQNPDASPVRLFTTYDRRTNVTQEHFSQTELLGRPYGTQRGVYLLTSHRTATAAEELAFLMQSLGWATLVGEITAGSLLHTHTVSLLETPEGGLALTVPVLTFIDNHGECWLGGGVVPDAIVLAEEALDKAWEVLEFHRNLGELVEGTGHLLEAHYARPEVVGQMGALLRAKLAQGAYRTAVDLESLASQLTADLQEMSGDHRLLVFHSPGEMVAEEVPPPPPVVPSPEELSYLIEALFKTEVLPGQLGYLRFDAMAELETVKAIGPQLVQLVWQKLVDTDSLVVDLRYNPGSYSTAVPLLCSYFFEAEPRQHLYSVFDRATSRVTEVWTQPHVTGQRYGSHKDLYVLVSHTSGSAAEAFAHTMQDLQRATIIGEPTAGGALSVGIYQVGSSPLYASMPTQMAMSASTGEAWDLAGVEPDITVPMSVALSTARDIVTLRAKVPTVLQTAGKLVADNYASPELGVKMAAKLSGLQSRYARVTSEASLAEMLQADLQVLSGDPHLKTAHIPEDAKDRIPGIVPMQIPSPEAFEDLIKFSFHTNVLEDNVGYLRFDMFADCELLTQVSELLVEHVWKKIVHTDALIVDMRFNIGGPTSSISALCSYFFDEGPPILLDKIYNRPNDSVSELWTLTQLEGERYGSKKSMVILTSSLTAGAAEEFTYIMKRLGRALVIGEVTSGGCQPPQTYHVDDTDLYITIPTARSVGAADGSSWEGVGVVPDVAVPADAALTRAKEMLQHTSLRARRSPRLHGRRKGHHRQNQGRAGSLGRNQGAVRHEVLTEAPNGQKRGLLHSG

SEQ ID NO: 303 - protein, 1236 aa, Meleagris gallopavo
MIGTHLFLFSVLIVCSISAEEIFQPTLVLDMAKILLDNYCYPENLVGMQEAIEQAIKSGEILDISDPKMLANVLTAGVQGALNDPRLIISYEPSPPAAAKQEAETYLTREQLLSLIEHIVIYDKLEGNVGYLRIDYVIGQEVVEKVGAFLVDKVWKTLINTSALVIDLRYSTGGQISGIPFIISYLHEADKMLHVETVYNRPSNTTTEVWTLPKVLGERYSKDKDVIVLISHHTTGVAEDVAYILKHMSRAIIVGEKTAGGSLDIQKLRIGPSNFYMMVPVSRSISPLSGGGQSWEVSGVMPCVASEAEQALQKSLDILAVRRAVPGTLSRLADILKDYYSLVERVPVLLRHLATSDFSSVQSVEDLATKLNTEMQTLSEDPRLLVRAMMPGETAALPAEMPIAMATNLPDNEQLLHALVDTVFKVSVLPGNVGYMRFDEFADASVLDKLGPYIVKKVWEPLQNTENMIMDLRYNPGGPSSSAVPVLISYFQDPTASPVHLFTTYDRRTNHTQEHNSQAELLAQTYGAQRGIYVLTSRHTATAAEEFAYLMQSLGRATLIGEITAGSLSHTRTFPLLQPEQGITCGLTITVPVITFIDNHGESWMGGGVVPDAIVLAEDALEKAEEVLAFHRKMGMLLESTGQLLEAHYAIPEVAEKASVMLNTKRLQGGYRSAVDFETLASQLTSDLQEASGDHRLHVFHSHVEPTLEEQLPNMIPSPEELSYIIDALFKIEVLPGNLGYLRFDMMAEAETVKAIGPQLVHIVWNKLVDTDAMIIDMRYNTGGYSTAVPILCSYFFEPEPRQHLYTVFDRSTSRSTEVWTLPKVTGKRYGSLKDIYILTSHLSGSAAEAFTRSMKDLHRATVIGEPTVGGSLSVGIYRVGNSSLYASIPSQVVLSPVTGKVWSVSGAEPHITIQASEALAVAQHIANLRAQVPQIVKTVGKLVAENYAFVDIGTDIASNLTESINKENYKRINSEKELARKLTAILQTLSDDEHLKMLYIPEHAKDSIPGIVPKEIPSPEVFEDLIKFSFHTNVLENNIGYLRFDMFGDCELLTQVSDLLVEHVWKKIVHTDALIIDMRYNIGGYTNSIPILCSYFFDEGHQVLLDKVYDRPSDSVKEIWTQPQLRGERYGSQKGLIILTSAVTAGAAEEFVFIMKRLGRALIIGEQTSGGSHSPQTYQVDDTNFYIIIPTAQSVISAESSSWEGKGVPPHMETPAVTALIKAKEVLSAHLHNSR

SEQ ID NO: 304 - protein, 1298 aa, Oryctolagus cuniculus
MTRWSLLLCTLLCGLAGPSHLFQPSLVLDMAKVLLDNYCFPENLMGMQAAIEQAMKSQEILSISDPETLAQVLTVGVQSSLNDPRLVISYEPSTLEVPRQAPAPSNLTQEELLAWLQKSIQHEILEHNVGYLRVDDLPGQEVLSKLGGFLVAHVWGQLMGTSALVLDLRHCTGGHVSGIPYVISYLHPGNTVVHVDTIYDRPSNTTTELWTLPQVLGERYSADKDVVVLTSGRTGGVAEDIAYILKQMRRAILVGEQTVGGALDLQKLRIGQSDFFLTVPVSRSLGPLGGGSQTWEGSGVLPCVRTPAEQALEKALAILTLRRALPGLVRRLQEALQEYYTLVDRVPALLHHLATMDFSTVVSEEDLVSKLNAGLQSVSEDPRLLVRAMGPRETASGLEAAVEDPPEAAPTIPQDEAAWQVLVDSVFQVAVLPGNVGYLRFDRFAEASVLEALGPQVLRQVWEPLQDTEHLIMDLRYNPGGPSSGLPLLLSYFQGPEDSPVRLFTTYDRRTNITQEHLSHTTLLGPRYGNQRGVYLLTSHRTATAAEEFAFLMQSLGWATLVGEITAGSLLHTCTVPLLGTPEGGLVLTVPVLTFIDNHGEGWLGGGVVPDAIVLAEEALDRAQEVLEFHRSLGALVEGTGHLLEAHYARPEVVGQASALLRAKLAQGAYRTAVDLESLASQLTADLQEVSGDHRLLVFHSPGELVAEEVPPPPPAVPSLEELSYLIEALFKTDVLPGRLGYLRFDAMAELETVKAIGPQLVQLVWQGLVDTAALVVDLRYNPGSYSSAVPLLCSYFFAAEPRQHLYSIFDRASSRLTEVWTLPQVAGQRYSPHKDLYILTSHTSGSAAEAFAHTMQDLQRATVIGEPTAGGALAVGIYQVGSSSLYASMPTQTALSATTGEAWDMAGVEPDVTVPMSEALSTAQDIVALRAKVPTVLQTAGKLVADNYASPELGAQVAAKLSGLQSRYSRVTSEVALAEMLGADLQLLSGDPHLKTAHIPEDAKDRIPGIVPMQIPSPEVFEDLIKFSFHTNVLEDNIGYLRFDMFGDCELLTQVSELLIEHIWQKIVHTDAMIMDMRSVSPAGSQPGTWQRSREGPGRLQGSYGIQGFRRVLAYKISYSQEINALSDTDTQNSEGFNIGGPTSSISAVCSYFFDEGPPILLDKIYSRPNDSISELWTHTRLAGERYGSKKPVAILTSSLTAGAAEEFTYIMKRLGRALVVGEVTSGGCQPPQTYHVDDTHLYITIPTARSVGAADGSSWEGVGVTPHVAVPADTALTRAKEMLQHSLLRVRRSPGL

SEQ ID NO: 305 - protein, 1286 aa, Ovis aries
MVRKWALLLPMLLCSLAGPAHLFQPSLVLDMAQVLLDNYCFPENLMGMQEAIEQAIKSHEILSISDPQTLAHVLTAGVQSSLNDPRLVISYEPGTLEAPPRAPAFTNLTLEEIIAGLQDGLRHEVLEGNVGYLRVDNIPGQEVMSKLRSFLVANVWRKLMNTSALVLDLRHCTGGHISGIPYVISYLHPGNTILHVDTIYDRPSNTTTEIWTLPEALGEKYSADKDVVVLTSSRTGGVAEDIAYILKQMRRAIVVGERTVGGALDLQKLRIGQSDFFLTVPVSRSLGPLGEGSQTWEGSGVLPCVGTPAEQALEKALAVLRLRRALPGVIQRLQEALREYYTLVDRVPALLSHLAATDLSSVVSEEDLVTKLNAGLQAVSEDPRLQVQVVRPKEASSGPEEEAEEPPETVPEVPEDEAVRRALVDSVFQVSVLPGNVGYLRFDSFADASVLEVLGPYILRQVWEPLQDTEHLIMDLRQNPGGPSSAVPLLLSYFQSPDASPVRLFTTYDRRTNVTQEHFSQTELLGRPYGSQRGVYLLTSHRTATAAEELAFLMQSLGWATLVGEITAGSLLHTHTVSLLETPKGGLALTVPVLTFIDNHGECWLGGGVVPDAIVLAEEALDRAQEVLEFHRSLGELVEGTGHLLEAHYARPEVVGQMGALLRAKLAQGAYRTAVDLESLASQLTADLQEMSGDHRLLVFHSPGEMVAEEVPPPPPAVPSPEELSYLIEALFKTEVLPGQLGYLRFDAMAELETVKAIGPQLVQLVWQKLVDTAALVVDLRYNPGSYSTAVPLLCSYFFEAEPRQHLYSVFDRATSRVTEVWTLPHVTGQRYGSRKDLYVLVSHTSGSAAEAFAHTMQDLQRATIIGEPTGGGALSVGIYQVGSSSLYASMPTQMAMSASTGEAWDLAGVEPDITVPMSVALSTARDIVTLRAKVPTVLQTAGKLVADNYASPELGVKMAAKLSGLQSRYARVTSEAALAELLQADLQALSGDPHLKTAHIPEDAKDRIPGIVPMQIPSPEAFEDLIKFSFHTNVLEGNVGYLRFDMFGDGELLTQVSELLVEHVWKKIVHTDALIVDMRFNIGGPTSSISALCSYFFDEGPPILLDKIYNRPSDSVSELWTLTQLEGERYGSKKSMVILTSSLTAGAAEEFTYIMKRLGRALVIGEATSGGCQPPQTYHVDDTDLYITIPTARSVGAADGRSWEGVGVAPDVAVPADAALARAKEMLQHAPLRARRSPRLRGRGKGHHRQSQGRAGSLGRNQGAVRHEVLTEAPSGQKQGLLHSG

SEQ ID NO: 306 - protein, 1211 aa, Salmo salar
MAVGRFSQALLVLFCHVLYSSSSFQPALVLDMAKILLDNYCFPENLVGMQEAIQQAINSGEILKISDRKTLASVLTAGVQGALNDPRLTVSFEPNFVPVMPPALPSLPTEQLIRLVRNSVKLEFLENNVGYLRIDRIIGEETAAKLGPLLRENIWTKVTHASSLIFDLRYSTAGELSGVPFIISYFSDPEPLIHIDTVFDRPSNTTKELWTMSSIMGERYGKRKDLIVLTSKRTMGAAEAIAYTLKHLNRAIIVGERSAGGSVKVQKIRIGDSGFYITVPVARSVNPITGQSWEVSGVSPSVNINAKEAVSNAKNLLAVRSAIPNAVQSVSDIIRQYYSFTDRVPALLQHLESTDFFSVISEEDLANKFNNELQSVSEDPRLMIKLTQDHPVIIEEDFEPENIPNDPAFLRNLVDTVFKVRILTGNTGYLRFDRFGDVSMMAKLGEQIAQKIWEPLKDTENLIIDLRYNTGGSSASMGLLLSFLQDPSQQFHFFTIYDRIQNTTTEFNTLSGLPGPPYGSKRGVYILTSYYTAGAGEEFAYLMQSLHRGTLIGEITSGTLMHSKSFQVEDTDIVITVPFINIIDNNGECWLGGGVVPDAIVLAEEAVDHTHEIIEFHREVRSLVEGTGELLEVHYAIPEVANKVSRVLLIKWAEGSYRSVVDYESLASQLTADLQETSGDHRLHIFYCDIEPESLHDMPKIPTAEEVGYIIDALFKIEVMPGNVGYFRFDMMADIEVMRAIGPELIKLVWSKLVNTDTMIIDMRYNTGGYSTAIPLLCTYFFDAEPLRHLYTIFDRSTATMTEVMTLPQIQGQRYGSTKDIYILTSHMTGSAAEAFTRTMKDLNRATVIGEPTIGGSLSSATYQIGNSILYASIPNQVVLSAVTGKSWEVAGVMPDVEVDAEDALAAAIKIINLRAEVPAILEATGALVADNYAFENVGADVAEKLAAMSGDYNLISSKMELEAKLSADLMTLSGDKCLKTTHNIPALPPMNPSPEMFIELIKVSFHTDLFENNIGYLRFDMFGDFEEVQAIAQIIVEHVWNKVVNTDALIVDLRNNVGGPTTAIAGFCSYFFDADKQVLLDKLYDRPSGTTKELWSLPELTGARYGTKKSLIILTSGATAGAAEEFVYIMKKHGRAMIVGETTNGASHPPETFRVGESEVFLSIPTTHSDTTQGPAWEGAGVAPHIPVPADAALDTAKGILNKHFAGAKK

SEQ ID NO: 307 - protein, 1284 aa, Sus scrofa
MPRKWALLLPMLLCSLTGPTHMFQSSLVLDTAKILLDNYCFPENLMGMQEAIEQATKSPEILAITDPQTLAHVLTAGVQNSLNDPRLIISYEPSTLEVTPKAPALRNLTLEELIEGLHNSLRHEVLEGNVGYLRVDDIPGQEVMNKLGSFLVVNVWEKLMGTSALVLDLRHCTRGHVSGIPYVISYLHPGNTVLHVDTVYDRPSNTTTEIWTLPEVLGDRYSADKDVVVLTSSHTGGVAEDIAYILKQMRRAIVVGERTVGGALDLQKLRIGQSDFFLTVPVSRSLGPLGEGSQTWEGSGVLPCVGTPAEQALEKALAILKLRRALPGVIQRLQEVLQEYYTLVDRVPALLHHLASMDLSSVVSEEDLVTKLNAGLQAVSEDPRLLVQVVKSKEPSSEPKDEAEEPPEVIPEVPEDEAARRALVDAVFQVSVLPGNVGYLRFDSFADASVLGVLAPYIVHQVWEPLQDTEHLIMDLRQNPGGPSTAVPLLLSYFQGPDPGPVRLFTTYDRRTNVTQEHYSHTELLGQRYSTQGGVYLLISHRTATAAEELAFLMQSLGWATLVGEITAGSLLHTHTVPLLETTEGGLSLTVPVLTFIDNHGECWLGGGVVPDAIVLAEEALDRAQEVLEFHRSLGELVEGTGHLLEAHYARPEVVGQTGALLRAKLAQGAYRTAVDLESLASQLTADLQEMSGDHRLLVFHSPGEMVAEEVPPPPPVVPSPEELSYLIEALFKTEVLPGRLGYLRFDAMAELETVKAIGPQLVQLVWQKLIDTAALVVDLRYNPGSYSTAVPLLCSYFFEAEPRQHLYSVFDRATSRVMEVWTVPQVAGQRYGPQKDLYILVSHTSGSAAEAFAHTMQDLQRATIIGEPTAGGALSVGIYQVGSSPLYASMPTQMALSASTGEAWDLAGVEPDITVPMSVALSTARDIVALRAKVPTVLQTAGKLVADNYASPEMGAKIAVKLSRLQSRYARVTSEASLAEMLEADLQLLSGDPHLKTAHIPEDAKDRIPGIVPMQIPSPEVFEDLIKFSFHTNVLEGNIGYLRFDMFGDRELLTQVSELLVEHVWKKIIHTDALIIDMRFNIGGPTSSISALCSYFFDEGPPILLDKIYNRPNDSVSELWTHTHLTGGSYGSKKSIIILTSSLTAGAAEEFTYIMKRLGRALVIGEVTSGGSQPPQTYHVDDTNLYITIPTARSVGAADGSSWEGVGVVPDVAVPAEGALARAQEMLQHTLLRARRSLRGQRLRRQRQGRAGPLGHIQRTLGHEVLTEAPKGWKRGLLHSSWA

SEQ ID NO: 308 - protein, 199 aa, Anas platyrhynchos
MAYTRRALPCLLLLALTFLGSSMAERDCRVSSFKVKENFDKNRYSGTWYAMAKKDPEGLFLQDNVVAQFTVDENGQMSATAKGRVRLFNNWDVCADMIGSFTDTDDPAKFKMKYWGVASFLQKGNDDHWVVDTDYDTYALHYSCRQLNEDGTCADSYSFVFSRDPKGLPPEAQKIVRQRQIDLCLDRKYRVIVHNGFCS

SEQ ID NO: 309 - protein, 201 aa, Bos taurus
MEWVWALVLLAALGSARAERDCRVSSFRVKENFDKARFAGTWYAMAKKDPEGLFLQDNIVAEFSVDENGHMSATAKGRVRLLNNWDVCADMVGTFTDTEDPAKFKMKYWGVASFLQKGNDDHWIIDTDYETFAVQYSCRLLNLDGTCADSYSFVFARDPSGFSPEVQKIVRQRQEELCLARQYRLIPHNGYCDGKSERNIL

SEQ ID NO: 310 - protein, 183 aa, Bos taurus
ERDCRVSSFRVKENFDKARFAGTWYAMAKKDPEGLFLQDNIVAEFSVDENGHMSATAKGRVRLLNNWDVCADMVGTFTDTEDPAKFKMKYWGVASFLQKGNDDHWIIDTDYETFAVQYSCRLLNLDGTCADSYSFVFARDPSGFSPEVQKIVRQRQEELCLARQYRLIPHNGYCDGKSERNIL

SEQ ID NO: 311 - protein, 201 aa, Cervus elaphus
MEWVWALVLLAALGSARAERDCRVSSFRVKENFDKARFAGTWYAMAKKDPEGLFLQDNIVAEFSVDENGHMSATAKGRVRLLNNWDVCADMVGTFTDTEDPAKFKMKYWGVASFLQKGNDDHWIIDTDYETYALQYSCRLLNLDGTCADSYSFVFARDPSGFSPEVQKIVRQRQEELCLARQYRLIPHNGYCDGKSERNTL

SEQ ID NO: 312 - protein, 196 aa, Gallus gallus
MAYTWRALLLLALAFLGSSMAERDCRVSSFKVKENFDKNRYSGTWYAMAKKDPEGLFLQDNVVAQFTVDENGQMSATAKGRVRLFNNWDVCADMIGSFTDTEDPAKFKMKYWGVASFLQKGNDDHWVVDTDYDTYALHYSCRELNEDGTCADSYSFVFSRDPKGLPPEAQKIVRQRQIDLCLDRKYRVIVHNGFCS

SEQ ID NO: 313 - protein, 196 aa, Meleagris gallopavo
MAYTRRSLLLLALAFLGSSMAERDCRVSNFKVKENFDKNRYSGTWYAMAKKDPEGLFLQDNVVAQFTVDENGQMSATAKGRVRLFNNWDVCADMIGSFTDTEDPAKFKMKYWGVASFLQKGNDDHWVVDTDYDTYALHYSCRELNEDGTCADSYSFVFSRDPKGLPPEAQKIVRQRQIDLCLDRKYRVIVHNGFCS

SEQ ID NO: 314 - protein, 201 aa, Oryctolagus cuniculus
MEWVWALVLLAALGSGRGERDCRVSSFRVKENFDKARFAGTWYAMAKKDPEGLFLQDNIVAEFSVDENGHMSATAKGRVRLLNNWDVCADMVGTFTDTEDPAKFKMKYWGVASFLQRGNDDHWIIDTDYDTFAVQYSCRLLNFDGTCADSYSFVFSRDPHGLPPDVQKLVRQRQEELCLSRQYRLIVHNGYCDDKSVRNLL

SEQ ID NO: 315 - protein, 201 aa, Sus scrofa
MEWVWALVLLAALGSAQAERDCRVSSFRVKENFDKARFSGTWYAMAKKDPEGLFLQDNIVAEFSVDENGHMSATAKGRVRLLNNWDVCADMVGTFTDTEDPAKFKMKYWGVASFLQKGNDDHWIIDTDYDTYAVQYSCRLQNLDGTCADSYSFVFARDPHGFSPEVQKIVRQRQEELCLARQYRIITHNGYCDGKSERNIL

SEQ ID NO: 316 - protein, 201 aa, Sus scrofa
MEWVWALVLLAALGSAQAERDCRVSSFRVKENFDKARFSGTWYAMAKKDPEGLFLQDNIVAEFSVDENGHMSATAKGRVRLLNNWDVCADMVGTFTDTEDPAKFKMKYWGVASFLQKGNDDHWIIDTDYDTYAAQYSCRLQNLDGTCADSYSFVFARDPHGFSPEVQKIVRQRQEELCLARQYRLITHNGYCDGKSERNIL

SEQ ID NO: 317 - protein, 192 aa, Thunnus albacares
MLRYVVALCLLALAWAQDCQVANFQVMQDFDRSRYAGTWYAVAKKDPEGLFLIDNIVAQFIVGEDGKMTATAKGRVIILNNWEMCADMLATFEETPDPAKFRMKYWGVSSYLQTGNDDHWVIDTDYDNYAIHYSCRLIDADGTCLDSYSFIFSRHPTGLRAEDQRIVTQKKMDLCLLGKYRRVAHNGFCETS

SEQ ID NO: 318 - protein, 135 aa, Anas platyrhynchos
MPPNLTGYYRFVSQENMDNYLRALDINVVLRKVVCLLKPDKEIIHTGDHMVIRTITSLRDYVMDFDLGVQFEEDLGPVDGRKCQTTVSWEGDQLVCEQLGEKRNRGWRHWLEGDRLHLRMTAEDEVCVQVFQKVK

SEQ ID NO: 319 - protein, 135 aa, Cervus elaphus
MPPDLTGYYRFISQKNLEDYLQALNINMALRKIALLLKPDKEIDQRGNHMTVKTLSTFRNYILEFEVGVEFEEDLRIVDGRKCQSIITWEGEQLVCVQKGEIPNRGWRLWLEGEMLYQEVTARDAVCQCIFRKVK

SEQ ID NO: 320 - protein, 135 aa, Gallus gallus
MVPNLSGYYRFVSQENMDNYLRALDINVVLRKVVCLLKPDKEIIHTGDHMVIRTITSLRDYVMDFDLGVQFEEDLGPVDGRKCQTTVFWEGDQLVCEQLGEKRNRGWRHWLEGDRLHLRMTAEDEVCVQVFQKVK

SEQ ID NO: 321 - protein, 135 aa, Meleagris gallopavo
MLPNLSGYYRFVSQENMDNYLRALDINVVLRKVVCLLKPDKEIIHTGDHMVIRTITSLRDYVMDFDLGVQFEEDLGPVDGRKCQTTVFWEGDQLVCEQIGEKRNRGWRHWLEGDRLHLRMTAEDEVCVQVFQKVK

SEQ ID NO: 322 - protein, 135 aa, Ovis aries
MPPNLTGYYQFVSQKNLEDYLQALNINMALRKIALLLKPDKEIDQQGNHMTVKTLSTFRNYILKFEVGVEFEEDLRIVDGRKCQTIVTWEEEQLVCVQKGEVPNRGWRLWLEGQMLHQEVTARDAVCQCVFRKVK

SEQ ID NO: 323 - protein, 249 aa, Gallus gallus
MRRVRETQKHISTVKYHEDFEKNKGTFTPVVTDPITERVKKNMHDFSDISYRGIQRRVVEMEQKRVDQDQENLTGLRVWRTNPGSVFDYDPAEDNIQSRSLHMISVQAQRRSREHSRSASALSISGGDEKSEPSEGVNQHLSYYSSGGFFTTTATVGYKHAKTIELPQQRSASVATQQTTVSSVPSHPSTAGKTYRAMYDYTAADADEVSFKDGDTIVNVQAIDEGWMYGTVQRTGKTGMLPANYVEAV

SEQ ID NO: 324 - protein, 409 aa, Cervus elaphus
MRAERTSFLLVLGLLLAGICSVHSLPENVMVKDQRRRVDDHTLASSNTDFAFSLYKKLALKNPNKNVIFSPLSVSIALAFLSLGARGSTLTEILEGLKFNLTEIQEKEIHQGFQHLLQTLNKPSNQLQLSVGNAMFVQEELELLDKFIEDARVLYASGAFSTDFRDSEAAKRLINEYVKNKTQGKIEELFKFLSPRTELVLVNYIYFKAQWKTPFDPKHTQQAEFHVSENKTVEVPMMSIGLETPYFRDEELGCTLVELTYTSNDSALLILPDEGKMQDLEAKLNPETLTRWRKSLQPRRIHELYLPKFSIKSDYELNDILSQLGIKKIFGGADLSGITGTADLVVSQVVHSAALDVDEEGTEGAAATGISMERTILRITVRFDRPFLIAIVLKDTQSIIFLGKVTNPS

SEQ ID NO: 325 - protein, 345 aa, Ovis aries
MSRSYNRCKKFQTKTPESVDHHDEASELEMRRTSNSCIIENGHQPGADPGDGPPETTQTIPGPEPPKPRPVSLSLRLPHQPVTAVTRVSERFSGETSATALSPTSAAILGGLGSSPSEATTPWTPSPSEKNSSVPRSLSSSGFGAMTASRSNDSPPLVTPPQSPPSPHPPATTQAHRPGERRRELVRSQTLPRTSGAQARKALFEKWEQDTAGKGKGETRAKLKRSQSFGVASASSIKQILLEWCRSKTLGYQHVDLQNFSSSWSDGMAFCALVHSFFPDAFDYNALSPTQRRQNFELAFTMARNLANCERLIEVEDMMVMGRKPDPMCVFTYVQSLYNHLRRFE

SEQ ID NO: 326 - protein, 167 aa, Ovis aries
MATSELSCQLSEENCERREAFWAEWKDLTLSTRPEEGCSLHEEDAQRHETYHRQGQCQALVQRSPWLVMRMGILGRGLQEYQLPYQRVLPLPIFTPAKVGATKEEREETPIQLQELLALETALGGQCVDRQDVAEITKQLPPVVPVSKPGTLRRSLSRSMSQEAQRG

SEQ ID NO: 327 - protein, 193 aa, Gallus gallus
MTTKAPTFTQPLQSVVALEGSAATFEAHISGFPVPEVSWYRDGQVLSAATLPGVQISFSDGRAKLVIPSVTEANSGRYTIQATNGSGQATSTAELLVTAGTAPPNFSQRLQSMTARQGSQVRLDVRVTGIPTPVVKFYRDGVEIQSSPDFQILQEGDLYSLIIAEAYPEDSGTYSVNATNNVGRATSTAELLI

SEQ ID NO: 328 - protein, 276 aa, Gallus gallus
MTEPVTLIRDIENQTVLTDEDAIFECEIKINYPEIKLSWYKGTQKLDSSDKYKIKIEGDRHILKIKNCQLEDQGNYRIVCGPHIASARLTVIEPAVERHLHDTTFKEGNTCTLSCQFSIPNAKSQWYRNGRPIKIGGRYSTQVSDKVHKLIIKDVRTEDQGQYTCKLDNLETTADLTIEAEPIQFTKSIQNIVVSEHQSATFECEVSFDDAVVTWYKGPTELTESPKYSFRSEGRCHYMTIHNVTAEDEGVYSVIARLEPRGEARSTAELYLVTKE

SEQ ID NO: 329 - protein, 540 aa, Gallus gallus
MEGEEEWEETYDKREVAYEEEQIIHEEVVEVPKKPVPERKPPAITAEKKEKKEVTVHKVVKKPVDEKVEITTQRVAEEKLKQAEVTKKIPSAKPTPMIIEEKVMKKEAHKEVEEEEEREVISEELETHHVEVPDVPKKIPEEKVPITVPKKPEPTPAKEPEVHKIIEEKIKVSEPKKPEVPPAKVPEVPKKVVKEEVSVEVPKKPEPPPAKVPEVPKKVVPEEKVHVEVPKKAEPPPPKVLRKKPEEEEPKPEKEPKPEVKPVPTPVEKIKKPEVPEVPKKKTEIPVIKKEEKHVPEPPKEPKPAAISVPGPEPKPAVALKSPKPAAEPAPAITTAPVTTPVVGKKAEAKPTKEETPKGISPVKAKKTPSPADAERRKLRPGSGGEKPPEEPPFTYQLKAVPLKFVKEMKDIVLKEAESVGSSAIFEVLISPSTAITSWMKDGSNIRESPKHKFIADGKDRKLHIIDVQLSDAGEYTCVLRLGNKEKTSTAKLIVEELPVRFVKTLEEEVTVVKGQPLYLTCELNKERSVVWRRDGKIIK

SEQ ID NO: 330 - protein, 596 aa, Gallus gallus
MVTTKQTVISRTREGIVTSQEQRHISREKVRKEPEKTPVPTVIIVTAKVKEQERISTAREEISARHEQVHVSHEQIRREDVKPSVPKVVITTDKPKAPVLISQSKEGIATKKEHVSISHEKIKKEAKKTTAVLKIIAPVTVSRTREEITAKPEQMHLAYDQIEAGKRAEAVATVVAAVDQARVRSPWETEQVDETYVKKKTLEYGYKEHAVKAHEAQAEHHVATKEVKTVYVPPEKHIPAAEKKEVHVSTEIKRETEAKIEKTNHIEHPRPRTASPHFTVSKIAVPKPDHTYEVSIAGSAMATLEKELSATSAEQKITKPVKPPQLKPHEVKIKPESAPPPFPFTEAAETYKAHYDVETKKEVDVSIKGGAVREDHLLLRKESEAKVTETARVPVPAEIPVTPTHLVWGLKNKTVTEGESVTLECHISGHPQPTVTWYRDDYKIESSMDFQITFKAGLARLVIREAFAEASGRFTCTATNKAGSVSTSCHLHVKVSEETETRETISEKVVTEEKSYVETQDVVMEDASPPPEEVSGEPVPPFFIRKPVVHTLIVGGSIIFEFHVGGNPKPHVLLKKGGVPLTTGYRYKVSYKRETG

SEQ ID NO: 331 - protein, 818 aa, Gallus gallus
MRESDHRAWTPVSYTVTRQNAVVQGLTEGKAYFFRIAAENIIGMGPFTETTKELIIRDPITVPDRPEDLEVKAVTKNSVTLTWNPPKYNGGSDITMYVLESRLIGKEKFHRVTKEKMLDRKYTVEGLKEGDTYEYRVSACNIVGQGKPSFCTKPITCKDEIAPPTLDLDFRDKLIVRVGEAFSLTGRYSGKPTPKITWLRDDIVLKEDDRTKIKTTPTTLCLGILKSVREDSGKYCVTVENSTGSRKGFCQVTVVDRPSPPVGPVIFDEVHKDHMIVSWKPPLDDGGSEITNYIIEKKDTHRDLWMPVTSATVKTTCKIPKLLEGREYQVRIYAENLYGISDPLISDEMKAKDRFSVPDAPEQPVIKDVTKDSAVVVWNKPYDGGKPITNYIVEKKETMATRWVRVTKDPIFPSTQFKVPDLLEGCQYEFRVSAENEIGVGNPSPPSKPIFARDPIVKPSPPVNPEAVDKTKNSVDLTWQPPRHDGNGKIIGYLVEYQKVGDEEWKKANLTPDSCPETKYKVTGLTEGLTYKFRVMAVNAAGESEPAYVPDPVEVKDRLEPPELILDANMAREQHVRAGDTLRLSAVIKGVPFPKVSWKKEDKEVSPKADIEVTGVGSKLEIRNTVHEDGGIYSLTVENPAGSKTVSVKVLVLDKPGPPRNLQVSDVRSDSCYLTWKEPEDDGGSVITNYVVERKDVASAQWVSVSSSSKKRSHMAKYLMEGTQYLFRVAAENLFGRGPYVETLKPIKAMDPLHPPGPPKNLHHIDVDKTEVSLVWNRPDRDGGAEITGYLVEYQEDGADEWTKFQTVPMLDCVVTGL

SEQ ID NO: 332 - protein, 254 aa, Ovis aries
MIAEDLGRGQFGIVHRCVETSSKKTYMAKFVKVKGTDQVLVKKEISILNIARHRNMLYLHESFESMEELVMIFEFLSGLDIFERINTSAFELNEREIVSYVRQVCEALEFLHSHNIGHFDIRPDNIIYQTRRSSTIKIIEFGQARQLKPGDNFRLQFTAPEYYAPEVHQHDVVSTATDMWSLGTLVYVLLSGINPFLAETNQQVIENIMNAEYTFDEEAFQEISLEAMDFVDRLLVKERKSRMTASEALQHPWL

SEQ ID NO: 333 - protein, 268 aa, Ovis aries
MSGEPSPEIEWFKNNLPISISSNISVSRSRNVYSLEIRNASVSDSGKYTIKAKNFRGQCSATASLTVLPLVEEPPREVVLRTSGDTSLQGSFSSQSVQMSASKQEASFSSFSSSSASSMTEMKFASMSAQSMSSMQESFVEMSSSSFMGKSSMTQLESSTSRMLKAGLRGIPPKIEALPPDISIDEGKVLTVACAFTGEPTPEITWSRGGRTIHDQEQQGRFHIENTDDLTTLIIMDVRKQDGGLYTLSLGNEFGSDSATVNINIRSI

SEQ ID NO: 334 - protein, 322 aa, Cervus elaphus
MSCLDVMYQVYGPPQPYFAAAYTPYHQKLAYYSKMQEAQECNASPSNSSGSGSSSFSSQTPASIKEEESSPEKERPPEAEYINSRCVLFTYFQGDISSVVDEHFSRALSQPSSYSPSCSSSKAPRSSGPWRDGSYPMSQRSFPASFWNSAYQTPVPAPLGSPLAAAHSELPFAAAAAADPYSPAALHGHLHQGAAEPWHHAHPHHAHPHHPYALGGALGAQAAAYPRPAAVHEVYAPHFDPRYGPLLMPAASGRPARLAPAPAPAPGSPPCELSAKGEPASAAWAAPGGPFASPAGDVPGGLGLSVDSGLQPQDKSKDLYWF

SEQ ID NO: 335 - protein, 200 aa, Gallus gallus
MANKGPAYGMSRDVQSKIEKKYDDELEDRLVEWIVAQCGSSVGRPDRGRLGFQVWLKNGIVLSQLVNSLYPDGSKPVKIPDSPPTMVFKQMEQIAQFLKAAEDYGVVKTDMFQTVDLFEAKDMAAVQRTLVALGSLAVTKNDGHYHGDPNWFMKKAQEHKREFSESQLKEGKNIIGLQMGTNKGASQAGMSYGRPRQIIS

SEQ ID NO: 336 - protein, 201 aa, Ovis aries
MANKGPSYGMSREVQSKIEKKYDEELEERLVEWIVVQCGPDVGRPDRGRLGFQVWLKNGVILSKLVNSLYPDGSKPVKVPENPPSMVFKQMEQVAQFLKAAEDYGVTKTDMFQTVDLFEGKDLAAVQRTLMALGSLAVTKNDGHYRGDPNWFMKKAQEHKREFTESQLQEGKHVIGLQMGSNRGASQAGMTGYGRPRQIIS

SEQ ID NO: 337 - protein, 345 aa, Cervus elaphus
MSSYQKELEKYRDIDEDEILKTLSPEELEQLDCELQEMDPENMLLPAGLRQRDQTKKSPTGPLDREALLQYLEQQALEVKERDDLVPFTGEKKGKPYIQPKREIPVEEQVTLEPELEEALAHATDAEMCDIAAILGMYTLMSNKQYYDAICSGEICNTEGISSVVQPDKYKPVPDEPPNPTNIEEILKSVRSNDKEVEEVNLNNIQDIPIPMLTELCEAMKTNTHVRSFSLVATRSGDPIANAVADMLRENRSLQSLNIESNFISSTGLMAVLKAVRENATLTELRVDNQRQWPGDAVEMEMATVLEQCPSIVRFGYHFTQQGPRARAAQAMTRNNELRRQQKKR

SEQ ID NO: 338 - protein, 284 aa, Bos taurus
MDAIKKKMQMLKLDKENALDRAEQAEADKKAAEDRSKQLEDELVSLQKKLKATEDELDKYSEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGKCAELEEELKTVTNNLKSLEAQAEKYSQKEDKYEEEIKVLSDKLKEAETRAEFAERSVTKLEKSIDDLEDELYAQKLKYKAISEELDHALNDMTSI

SEQ ID NO: 339 - protein, 284 aa, Gallus gallus
MDAIKKKMQMLKLDKENALDRAEQAEADKKAAEERSKQLEDELVALQKKLKGTEDELDKYSESLKDAQEKLELADKKATDAESEVASLNRRIQLVEEELDRAQERLATALQKLEEAEKAADESERGMKVIENRAQKDEEKMEIQEIQLKEAKHIAEEADRKYEEVARKLVIIEGDLERAEERAELSESKCAELEEELKTVTNNLKSLEAQAEKYSQKEDKYEEEIKVLTDKLKEAETRAEFAERSVTKLEKSIDDLEDELYAQKLKYKAISEELDHALNDMTSI

SEQ ID NO: 340 - protein, 284 aa, Ovis aries; identical to SEQ ID NO: 338

SEQ ID NO: 341 - protein, 284 aa, Cervus elaphus
MEAIKKKMQMLKLDKENALDRAEQAEAEQKQAEERSKQLEDELAAMQKKLKGTEDELDKYSEALKDAQEKLELAEKKAADAEAEVASLNRRIQLVEEELDRAQERLATALQKLEEAEKAADESERGMKVIENRALKDEEKMELQEIQLKEAKHIAEEADRKYEEVARKLVIIEGDLERTEERAELAESKCSELEEELKNVTNNLKSLEAQAEKYSQKEDKYEEEIKILTDKLKEAETRAEFAERSVAKLEKTIDDLEDELYAQKLKYKAISEELDHALNDMTSI

SEQ ID NO: 342 - protein, 284 aa, Cervus elaphus
MDAIKKKMQMLKLDKENAIDRAEQAEADKKQAEDRCKQLEEEQQALQKKLKGTEDEVEKYSESVKDAQEKLEQAEKKATDAEADVASLNRRIQLVEEELDRAQERLATALQKLEEAEKAADESERGMKVIENRAMKDEEKMELQEMQLKEAKHIAEDSDRKYEEVARKLVILEGELERSEERAEVAESKCGDLEEELKIVTNNLKSLEAQADKYSTKEDKYEEEIKLLEEKLKEAETRAEFAERSVAKLEKTIDDLEDEVYAQKMKYKAISEELDNALNDITSL

SEQ ID NO: 343 - protein, 284 aa, Gallus gallus
MEAIKKKMQMLKLDKENAIDRAEQAEADKKQAEDRCKQLEEEQQGLQKKLKGTEDEVEKYSESVKEAQEKLEQAEKKATDAEAEVASLNRRIQLVEEELDRAQERLATALQKLEEAEKAADESERGMKVIENRAMKDEEKMELQEMQLKEAKHIAEEADRKYEEVARKLVVLEGELERSEERAEVAESKCGDLEEELKIVTNNLKSLEAQADKYSTKEDKYEEEIKLLGEKLKEAETRAEFAERSVAKLEKTIDDLEDEVYAQKMKYKAISEELDNALNDITSL

SEQ ID NO: 344 - protein, 160 aa, Cervus elaphus
MTDQQAEARSYLSEEMIAEFKAAFDMFDADGGGDISVKELGTVMRMLGQTPTKEELDAIIEEVDEDGSGTIDFEEFLVMMVRQMKEDAKGKSEEELAECFRIFDRNADGYIDAEELAEIFRASGEHVTDEELESLMKDGDKNNDGRIDFDEFLKMMEGVQ

SEQ ID NO: 345 - protein, 163 aa, Gallus gallus
MASMTDQQAEARAFLSEEMIAEFKAAFDMFDADGGGDISTKELGTVMRMLGQNPTKEELDAIIEEVDEDGSGTIDFEEFLVMMVRQMKEDAKGKSEEELANCFRIFDKNADGFIDIEELGEILRATGEHVTEEDIEDLMKDSDKNNDGRIDFDEFLKMMEGVQ

SEQ ID NO: 346 - protein, 161 aa, Gallus gallus
MDDIYKAAVEQLTEEQKNEFKAAFDIFVLGAEDGCISTKELGKVMRMLGQNPTPEELQEMIDEVDEDGSGTVDFDEFLVMMVRCMKDDSKGKTEEELSDLFRMFDKNADGYIDLEELKIMLQATGETITEDDIEELMKDGDKNNDGRIDYDEFLEFMKGVE

SEQ ID NO: 347 - protein, 160 aa, Sus scrofa
MTDQQAEARSYLSEEMIAEFKAAFDMFDADGGGDISVKELGTVMRMLGQTPTKEELDAIIEEVDEDGSGTIDFEEFLVMMVRQMKEDAKGKSEEELAECFRIFDRNADGYIDAEELAEIFRASGEHVTDEELESLMKDGDKNNEGRIDFDEFLKMMEGVQ

SEQ ID NO: 348 - protein, 182 aa, Cervus elaphus
MGDEEKRNRAITARRQHLKSVMLQIAATELEKEVGRREAEKQNYLSEHCPPLHLPGSMSEVQELCRQLHAKIDAAEEEKYDMEVKVQKSTKELEDMNQKLFDLRGKFKRPPLRRVRMSADAMLKALLGSKHKVCMDLRANLKQVKKEDTEKERDLRDVGDWRKNIEEKSGMEGRKKMFETES

SEQ ID NO: 349 - protein, 188 aa, Gallus gallus
MPEPRERKSKITASRKLLLKSLMLAKAKEEWEQEIVDKQSEKERYLSERITPLHTSGLSLSQLQDLCRELHEKVEIVDEERYDIEAKCNHNTREIKDLKLKVLDLRGKFKRPPLRRVRVSADAMLRALLGSKHKVSMDLRANLKSVKKEDTEKERPVEVGDWRKNVEAMSGMEGRKKMFDAAKSPTGQ

SEQ ID NO: 350 - protein, 265 aa, Gallus gallus
MSEAEEEYEEEQPEEEEEAAAAAEEEEEEEAEASKPHEEPEEERPRPRPVVPQLAPPKIPEGERVDFDDIHRKRMEKDLLELQTLIDAHFEQRRREENELVALMERIERRRAERNEQLRSRTEKERERQARLAEEKLRKEEEEAKKRAEDDAKKKKVLSNMPHFGGYLAKAEQRRGKRQTGREMKLRILAERKKPLHIEHMREDELRAKAKELHDWIQQLESEKFDLMEKLRRQKYEINVLYNRISHAQKFKKVVGKGRVGGRWK

SEQ ID NO: 351 - protein, 287 aa, Gallus gallus
MSDTEEVEHGEAHEAEEVHEEAHHEEAHHEEAHHEEAHHAEAHHAEAHHEEAHAHAEEVHEPAPPPEEKPRIKLTAPKIPEGEKVDFDDIQKKRQNKDLIELQALIDSHFEARRKEEEELVALKERIEKRRAERAEQQRIRAEKEKERQARLAEEKARREEEDAKRKAEDDLKKKKALSSMGASYSSYLAKADQKRGKKQTARETKKKVLAERRKPLNIDHLNEDKLRDKAKELWDWLYQLQTEKYDFAEQIKRKKYEILTLRCRLQELSKFSKKAGAKGKVGGRWK

SEQ ID NO: 352 - protein, 346 aa, Ovis aries; identical to SEQ ID NO: 13

SEQ ID NO: 353 - protein, 132 aa, Cervus elaphus; identical to SEQ ID NO: 225

(19) United States
(12) Patent Application Publication
(10) Pub. No.: US 2026/0191247 A1
(43) Pub. Date: Jul. 9, 2026

(54) COMPOSITIONS AND METHODS FOR ENHANCED EXPRESSION OF HETEROLOGOUS POLYPEPTIDES

(71) Applicant: BOND PET FOODS, INC., Boulder, CO (US)

(72) Inventors: Karin Pernilla TURNER AUDIBERT, Golden, CO (US); José Ginés GARCÍA-CERDÁN, Boulder, CO (US); Annabel ALONSO, Firestone, CO (US); Luis N. BRANDAO, Denver, CO (US); Karl Paul GERHARDT, Boulder, CO (US); Patrick John WESTFALL, Berthoud, CO (US); Raul REVELES, Boulder, CO (US)

(21) Appl. No.: 19/572,282

(22) Filed: Mar. 19, 2026

Related U.S. Application Data
(63) Continuation of application No. PCT/US2024/047875, filed on Sep. 20, 2024.
(60) Provisional application No. 63/539,586, filed on Sep. 20, 2023.

(51) Int. Cl. A23L 31/10; A23J 1/18; C07K 14/47; C12N 15/81

(52) U.S. Cl. CPC A23L 31/10; A23J 1/18; C07K 14/47; C12N 15/81

(57) ABSTRACT

Disclosed are methods and compositions for enhanced expression of a heterologous polypeptide of interest in an organism. The methods generally comprise first transforming the organism with a genetic construct having a heterologous polypeptide of interest and amplifying expression of the polypeptide through in vivo gene amplification.

### CROSS-REFERENCE TO RELATED APPLICATIONS

This application is a continuation of International Application Number PCT/US2024/047875, filed on Sep. 20, 2024, which claims the benefit of priority to U.S. Provisional Patent Application No. 63/539,586, filed Sep. 20, 2023, both of which is herein incorporated by reference in its entireties.

### INCORPORATION BY REFERENCE OF SEQUENCE LISTING

The present application is being filed along with a Sequence Listing in electronic format. The Sequence Listing is provided as a file entitled 68461-703.301_SL.xml, created Mar. 11, 2026, which is 420,162 bytes in size. The information in the electronic format of the Sequence Listing is incorporated by reference in its entirety.

### BACKGROUND OF INVENTION

Recombinant protein production is employed for production of various proteins/polypeptides useful as therapeutics, and in the food, detergent, paper, chemical, and cosmeceutical industries. Recombinant protein production in many cases replaces extraction of proteins/polypeptides from natural sources. Recombinant production of proteins/polypeptides can improve the yield and purity of protein/polypeptide products, increase the availability of proteins/polypeptides from scarce natural sources and decrease the cost of such products. Recombinant protein production can also provide unique protein/polypeptides (e.g., fusion proteins) that are not available from natural sources. Furthermore, recombinant protein production can also provide cost-effective modified proteins/polypeptides that have improved properties over proteins/polypeptides available from natural sources.

The disclosure relates at least in part to methods for robust or enhanced protein/polypeptide production in host organisms. Production of heterologous proteins/polypeptides in host organisms, particularly in yeast, at levels of about 20% of total protein. Enhancing the levels of proteins/polypeptides produced by fermentation in host organisms, such as yeast and filamentous fungi can result in significant decreases in costs for various protein and polypeptide products. There is a continuing need for lower cost production of proteins and polypeptides in a range of pharmaceutical, cosmetic, food and industrial applications. There is also a need in the art for the generation of alternative and new proteins/polypeptides.

One area of significant current interest is the use of recombinant protein/polypeptide production to provide alternative sources of proteins for use in food products. In particular, recombinant protein production can be used to generate proteins/polypeptides that function as a replacement for protein derived from animal meat.

The disclosure relates, at least in part, to the production of heterologous protein/polypeptides for use as protein in food compositions. The production of high-quality protein foods and feed typically includes animal meat. As the global human and companion animal populations increase, the demand for high-quality protein food is expected to increase. However, obtaining proteins from animal meat for food production is an environmentally demanding, and potentially destructive, process. While plant sources, e.g. legumes, contain a significant amount of protein, they often lack one or more essential amino acids for many mammalian diets or they are not as bioavailable as animal protein, making plant protein an insufficient or sub-optimal alternative for many food applications. In fact, tryptophan and lysine are scarce in corn, lysine in wheat and other cereals, and methionine in soybeans and other legumes. In addition, plant sources also contain anti-nutritional factors like fiber, phytate, and protease inhibitors that can limit digestion and absorption. Soybean, a commonly used protein source, decreases digestibility in canine foods when present in concentrations over 15%. Moreover, humans and companion animals have different amino acid requirements. Hence, there is a continuing need for a source of protein other than animal meat, which satisfies the growing demand for high-quality protein food products and delivers on a multitude of nutritional needs.

### SUMMARY OF THE INVENTION

In an aspect, the invention provides a food ingredient composition comprising a recombinant fungi, wherein the genome of the recombinant fungi comprises at least 2 copies of a tandem amplification region integrated into a locus of a haploinsufficient gene tied to fungi fitness, wherein the tandem amplification region comprises: a coding sequence (CDS) of one or more heterologous animal proteins in operable linkage with a first promoter and a terminator, wherein the one or more heterologous animal proteins: have less than 25% amino acid sequence identity to proteins endogenous to said fungi; and are characterized by an average molecular weight between 5 kDa and 80 kDa; at least 5% of the one or more heterologous animal protein by dry cell weight; and at least 50% total protein by dry cell weight.

Further disclosed herein is a food ingredient composition comprising recombinant fungi, wherein: (a) the recombinant fungi is transformed with a vector that creates at least 2 copies of a tandem amplification region integrated into the genome of the fungi at a locus of a haploinsufficient gene tied to fungi fitness, wherein the tandem amplification region: expresses one or more heterologous animal proteins, wherein the one or more heterologous animal proteins comprise: 1. less than 25% amino acid identity with proteins endogenous to said fungi; and 2. an average molecular weight of between 5 kDa and 80 kDa; (b) the composition comprises at least 5% of the one or more heterologous animal protein by dry cell weight; and (c) the composition comprises at least 50% total protein by dry cell weight.

The present invention further includes a recombinant fungi, wherein the genome of the recombinant fungi comprises at least 2 copies of a tandem amplification region integrated into a locus of a haploinsufficient gene tied to fungi fitness, wherein the tandem amplification region comprises: a coding sequence (CDS) of one or more heterologous animal proteins in operable linkage with a promoter and a terminator, wherein the one or more heterologous animal proteins: have less than 10% amino acid sequence identity with proteins endogenous to said fungi; and is characterized by an average molecular weight between 5 kDa and 80 kDa.

In an aspect, the invention provides a method for making heterologous polypeptides of interest in fungi for use in food compositions, the method comprising: introducing one or more genetic constructs comprising a gene of interest encoding a heterologous annexin-like protein (ALP) or a fatty acid binding-like protein (FLP) or a retinoid binding protein into the genome of a fungus to generate one or more transformed fungal strains; wherein the one or more transformed fungi strains comprise a heterologous polypeptides of interest content of at least 2% of the total protein content of the transformed fungal strains; thereby making heterologous polypeptides of interest in fungi for use in food compositions.

Further disclosed herein is a method for generating transformed yeast strains capable of enhanced expression of one or more heterologous polypeptides of interest, the method comprising: providing at least one genetic construct comprising: a first promoter homologous to at least a portion of the native promoter region of a haploinsufficient gene tied to yeast growth fitness; an auxotrophic marker; a gene of interest encoding a heterologous polypeptide of interest; and a synthetic open reading frame (ORF) homologous to at least a portion of a native ORF of the haploinsufficient gene tied to yeast growth fitness; integrating the at least one genetic construct into the haploinsufficient gene tied to yeast growth fitness to produce one or more transformed yeast strains, wherein the integrating generates a tandem amplification region; and screening the one or more transformed yeast strains based on (i) auxotrophy; (ii) yeast growth fitness; (iii) total protein content, and (iv) heterologous polypeptide of interest content and an average gene copy number of the coding sequence of the heterologous polypeptide of interest; selecting the transformed yeast strains having a heterologous polypeptide of interest content of at least 2% of the total protein content; thereby generating transformed yeast strains capable of enhanced expression of one or more heterologous polypeptides of interest. In certain aspects, the gene of interest is an ortholog gene of interest functionally analogous to the CDS of a selected native gene of interest. In some aspects, the method further comprises deleting the CDS of the selected native gene of interest from a deletion site of the yeast genome; wherein the deleting step is performed prior to, or simultaneously with, the integrating step.

In some aspects, the invention provides use of any one of the methods described herein to make a food composition.

In some other aspects, the invention provides a pharmaceutical composition comprising at least one of the transformed fungi strains or the transformed yeast strains of any one of compositions or methods described herein and a pharmaceutically acceptable carrier.

The present invention further includes a medicament comprising at least one of the transformed fungi strains or the transformed yeast strains of any of the compositions or methods described herein.

Without wishing to be bound by any particular theory, there may be discussion herein of beliefs or understandings of underlying principles relating to the compositions and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1A. A schematic of an exemplary RPL25-based genetic construct for integration into the genome of S. cerevisiae for in vivo amplification of AnnexinA3 (AnxA3). The genetic structure for gene amplification in S. cerevisiae for the HapAmp expression of AnnexinA3 (AnxA3), as modified from Peng et al. (2022). This structure has recombination arms at each end. Arm 1 is homologous to the promoter region of a haploinsufficient gene (element #1), and Arm 2 is homologous to the haploinsufficient gene open reading frame (element #2). This allows insertion of the construct into the genome by homologous recombination. Downstream of Arm 1 is an auxotrophic selectable marker selection (URA3 gene) for transformation and homologous Arm 3, which is homologous to the terminator region of the haploinsufficient gene (element #3). Between Arm 3 and Arm 2, there is an autonomous replicating sequence (ARS max) and the BTS1 promoter. The BTS1 promoter is weaker than the native promoter of the haploinsufficient gene and positioned such that integration results in substitution of the native promoter of the haploinsufficient gene with the weaker promoter. To express heterologously AnxA3, the expression cassette containing AnxA3, the yeast TDH3 promoter and CYC1 terminator are inserted between Arm 3 and the weaker BTS1 promoter. Driving expression through a weaker promoter attenuates the protein yield from each copy of the RPL25 haploinsufficient gene. This, in turn, is expected to decrease the growth rate of S. cerevisiae. Native gene amplification of the region between homologous Arm 3 and the native terminator (element #3) will then occur as this yeast evolves towards faster growth.

FIG. 1B. A schematic of an exemplary RPL25-based genetic construct for integration into the genome of S. cerevisiae for in vivo amplification of AnnexinA3 (AnxA3). This schematic differs from FIG. 1A in one aspect: the absence of ARS Max from between Arm 3 and Arm 2.

FIGS. 2A-2C. Integration and construction of integration plasmid examples. FIG. 2A depicts pBOND810, an example of a RPL25-based plasmid for haploinsufficient amplification in S. cerevisiae. FIG. 2B depicts pBOND827, an example of a RPL25-based plasmid for haploinsufficient amplification in S. cerevisiae. FIG. 2C depicts pBOND865, an example of a RPL25-based plasmid for haploinsufficient amplification in S. cerevisiae. For each of the plasmids depicted in FIGS. 2A-2C, the plasmids contain a S. cerevisiae origin of replication CEN/ARS for episomal plasmid maintenance; the bacterial ColE1 origin of replication, and the bacterial ampicillin selectable marker; S. cerevisiae native auxotrophic selectable marker URA3, which may be recycled via Cre-loxP system (Cre recombinase plus loxP sites); RPL25 HapAmp based gene amplification. As shown by the arrows in FIGS. 2A-2C, each of the plasmids may be linearized with NruI, PmeI and ZraI restriction enzymes prior to chromosomal integration, which allows for the removal of CEN/ARS, the bacterial ColE1 origin of replication, and the ampicillin selectable marker. pBOND810 of FIG. 2A is capable of driving heterologous expression of AnxA3. pBOND827 of FIG. 2B is capable of driving heterologous expression of AnxA3:GFP. In FIG. 2A and FIG. 2B, the cloning of the gene of interest, the promoter, and terminator are performed via Eagl and XhoI restriction enzymes. For the pBOND865 plasmid of FIG. 2C, the cloning of the gene of interest is performed via Golden Gate compatible SapI sites, and a second protein expression cassette may be engineered at the BsaI sites for dual heterologous protein expression.

FIGS. 3A-3D. Characterization of selected amplified AnxA3:GFP clones with variable fluorescence intensities. In each of FIGS. 3A-3D, the numbers (e.g., #31, #27, #7, etc.) correspond to an identification number of an AnxA3:GFP clone. FIG. 3A depicts the quantification of the fluorescence strengths of each AnxA3:GFP clone. FIG. 3B provides copy number analyses as performed by dPCR. FIG. 3C depicts the results from SDS-PAGE analysis. FIG. 3D depicts the protein content analysis of selected AnxA3:GFP clones. High GFP fluorescence strongly correlates with robust protein expression and high copy number. Clone #31 is a AnxA3:GFP “jackpot”.

FIGS. 4A-4B. Use of ImageJ fluorescence analyses to study frequency histograms of fluorescent AnxA3:GFP clones obtained with linearized pBOND827 construct. Frequency histograms, as shown in FIG. 4B, exhibit bimodal distributions with no or low fluorescent clones, clustered to the left, and mid to high fluorescent ones clustered to the right. Highly fluorescent amplified AnxA3:GFP clones (as indicated in FIG. 4A) are identified as “jackpots”, and their frequencies histograms are identified by the box on the right-hand side of FIG. 4B.

FIG. 5. SDS-PAGE protein content analysis of 10 AnxA3 clones transformed with linearized pBOND810. Note the variable AnxA3 protein expression among HapAmp clones. Clone S7 correspond to AnxA3 Jackpot #7.

FIGS. 6A-6C. 100 generations stability study of AnxA3 jackpot clone S7. FIG. 6A depicts growth curve over 16 days (~100 generations) in synthetic defined media. Yeast culture is grown for 24 hours and then diluted to OD600~0.2 for 16 days. FIG. 6B depicts SDS-PAGE AnxA3 protein content and copy number analysis as determined by dPCR. FIG. 6C depicts the gene copy number measurement from day 1 and day 16. AnxA3 protein expression and copy numbers are not significantly affected after 100 generations.

FIG. 7. Haploinsufficiency gene amplification rescue (HARES) of S. cerevisiae TDH3 haploinsufficient gene with animal orthologs. HARES can be implemented with any S. cerevisiae haploinsufficient locus. The genetic structure of HARES-TDH3 has recombination arms at each end. Arm 1 is homologous to the TDH3 promoter region (element #1), and Arm 2 is homologous to the TDH3 gene open reading frame (element #2). This allows insertion of the construct into the genome by homologous recombination. To promote rescue with the overexpressed animal ortholog protein during gene amplification, a stop codon or point mutations reducing the function of native TDH3 are engineered in Arm2. Downstream of Arm 1 are a selectable marker for transformation selection (URA3 gene) and homologous Arm 3, which is homologous to the terminator region of the TDH3 gene (element #3). Between Arm 3 and Arm 2, there is the BTS1 promoter. The BTS1 promoter is weaker than the native promoter and positioned such that integration results in substitution of the native promoter. To express heterologously TDH3 animal orthologs, the expression cassette containing the ortholog gene of interest, the yeast PGK1 promoter and PRM9 terminator are inserted between Arm 3 and the BTS1 promoter. Driving expression of TDH3 through a weaker promoter plus the introduced loss-of-function TDH3 variants, attenuates the protein yield from each copy of the yeast TDH3 haploinsufficient gene. This, in turn, is expected to decrease the growth rate in yeast. Gene amplification of the region between homologous Arm 3 and the native terminator (element #3) will then occur as yeast evolves towards faster growth.

FIG. 8A-8B. HARESCO (HapAmp rescue co-transformation A+B). HARESCO combines co-transformation experiment in S. cerevisiae by combining (A) specific targeted deletion of gene of interest (GOI) (e.g., TDH3) by homology recombination with a recyclable selectable marker (G-418), with (B) HapAmp RPL25-based for gene amplification of the animal ortholog. HARESCO can be implemented at any functional HapAmp site.

FIG. 9. Example of Results Obtained from Modified HapAmp Screening. Jackpots are enriched in the tiny-tiny category of clones obtained from a typical HapAmp transformation. Clones exhibiting slow growth and small colony size after 7 days are more likely to be HapAmp jackpots.

FIG. 10. Plasmid map depicting pBOND433. Backbone for the ANXA3 expression cassette provides the TEF1 promoter and IDP1 terminator DNA elements for plasmid pBOND455.

FIG. 11. Plasmid map depicting pBOND455. The ANXA3 expression cassette is flanked by the restriction sites XhoI and EagI and can be transferred to the POT1 expression plasmid pBOND308.

FIG. 12. Plasmid map depicting pBOND308. POT1 expression backbone vector contains a 2-micron origin of replication and the S. pombe tpi1+ gene for growth on glucose selection. The ANXA3 expression cassette can be inserted into the plasmid using the indicated XhoI and EagI restriction sites.

FIG. 13. Plasmid map depicting pBOND553. ANXA3 expression vector contains a 2-micron origin of replication, the S. pombe tpi1+ gene for growth on glucose selection, and the ANXA3 expression cassette. The Ampicillin resistance cassette, used for plasmid propagation in E. coli, is flanked by two SphI restriction sites, and can be removed by digestion with the SphI enzyme. The resulting large DNA fragment, with two direct repeat homologous sequences, is isolated and transformed into the S. cerevisiae host strain.

FIG. 14. Analysis of protein expression by SDS-PAGE. Protein extracts (10 and 15 μg of total protein) from timepoints of two independent fermentation runs of strain S2 (lanes A-D) vs. an empty vector control strain (lanes E). Strong expression of the ANXA3 protein (indicated by the white arrow) is detected in strain S2 but not in the control strain. (MWM—molecular weight marker).

FIG. 15. ANXA3 expression cassette linear DNA construct. It contains a 2-micron origin of DNA replication, the S. pombe tpi1+ gene for growth on glucose selection, and the ANXA3 expression cassette. The two direct repeat homologous sequences on both ends of the DNA fragment are in the same orientation. Homologous recombination of the homologous sequences results in circularization of the DNA fragment into a 2-micron high copy plasmid.

FIGS. 16A-16B. Annexin A3 dual expression cassette integration construct. FIG. 16A. Full length integration construct indicating the two ANXA3 expression cassettes, one driven by the TEF1 promoter and the other driven by the TDH3 promoter, the URA3 selectable marker, and the 5′ and 3′ regions of genome homology for integration at a safe-harbor locus. FIG. 16B. Schematic of the overlapping gene blocks used to assemble the integration construct of FIG. 16A.

FIG. 17. Analysis of protein expression by SDS-PAGE. Protein extracts (20 μg of total protein) from strains S5 and S6 vs. the parental strain S4. Expression of the ANXA3 protein (indicated by the white arrow) is detected in strains S5 and S6 but not in the control strain. (MWM—molecular weight marker).

FIGS. 18A-18C. Overall exemplary flowchart describing methods which may be employed to generate yeast strains capable of enhanced expression of a heterologous polypeptide of interest. FIG. 18A provides the exemplary methodology for gene selection as well as the first portion of the 2-micron plasmid screen. FIG. 18B provides the second portion of the 2-micron plasmid screen. FIG. 18C provide the third portion of the 2-micron plasmid screen as well as the three exemplary methods which may be employed to achieve enhanced expression.

FIGS. 19A-19C. Overall exemplary flowchart describing methods which may be employed to implement the modified HapAmp method. FIG. 19A provides the first portion, FIG. 19B provides the second portion, and FIG. 19C provides the third portion of the modified HapAmp method.

FIGS. 20A-20C. Overall exemplary flowchart describing methods which may be employed to implement the POT1 method. FIG. 20A provides the first portion, FIG. 20B provides the second portion, and FIG. 20C provides the third portion of the POT1 method.

FIGS. 21A-21C. Overall exemplary flowchart describing methods which may be employed to implement the SafeHarbor method. FIG. 21A provides the first portion, FIG. 21B provides the second portion, and FIG. 21C provides the third portion of the SafeHarbor method.

FIG. 22. Plasmid map depicting pBOND792.

FIGS. 23A-23D. Exemplary gels for 2-micron screens. FIG. 23A depicts SDS-PAGE and Anti-flag gels of certain lamb proteins expressed in transformed S. cerevisiae. For each of the gels, the columns are numbered 1-10, wherein 1: MW marker; 2: Empty vector (negative control); 3: Alpha actinin 3_Lamb; 4: Alpha-alpha skeletal muscle_Lamb; 5: Nebulin-like domain_Lamb; 6: Titin protein kinase domain_Lamb; 7: Titin M10 domain_Lamb; 8: Annexin A2_Lamb; 9: Annexin A1_Lamb; 10: Annexin A4_Lamb. FIG. 23B depicts a SDS-PAGE and Anti-flag gels of certain proteins expressed in transformed S. cerevisiae. For each of the gels, the columns are numbered 1-7, wherein 1: MW marker; 2: Annexin A3_Lamb; 3: Empty vector (negative control); 4: Park7_Box; 5: CycGly-richprot3_Box; 6: Annexin A4_Chicken; 7: Tropomyosin beta chain_Chicken. FIG. 23C depicts SDS-PAGE and Anti-flag gels of certain proteins expressed in transformed S. cerevisiae. For each of the gels, the columns are numbered 1-6, wherein 1: MW marker; 2: Annexin A3_Lamb; 3: Empty vector (negative control); 4: Fascin_Chicken; 5: Annexin A13 isoform X1_Chicken; 6: Gelsolin_Chicken. FIG. 23D depicts SDS-PAGE and Anti-flag gels of certain proteins expressed in transformed S. cerevisiae. For each of the gels, the columns are numbered 1-15, wherein 1: MW marker; 2: Empty vector (negative control); 3: Myosin motor domain_Chicken; 4: Titin 1-94A_Chicken; 5: Titin 4622-4896AA_Chicken; 6: SH3 Domain_Chicken; 7: Nebulin-like domain_Chicken; 8: Dystrophin_Chicken; 9: Annexin A6_Chicken; 10: Annexin A1_Chicken; 11: Transgelin_Chicken; 12: PGK1_Chicken1; 13: Pyruvate kinase domain_Chicken; 14: Beta-actin_Chicken; 15: Transgelin_Lamb.

FIGS. 24A-24D. Schematic views of the structures of RPL7 (FIG. 24A), RPL33 (FIG. 24B), RPN11 (FIG. 24C), and RPB7-based (FIG. 24D) genetic constructs for integration into the genome of S. cerevisiae for in vivo amplification of a fluorescent AnxA3:GFP reporter protein. RPL33, RPL17, RPB7, and RPN11 are haploinsufficient loci.

FIGS. 25A-25D. ImageJ fluorescence analysis of transformation plates obtained from transformation with linearized RPL33 (FIG. 25A), RPL17 (FIG. 25B), RPB7 (FIG. 25C), and RPN11 (FIG. 25D) AnxA3:GFP HapAmp constructs. Frequency histograms generated from fluorescence intensity characterization of S. cerevisiae AnxA3:GFP clones yielded jackpots with a frequency of less than 5% for RPL33, RPB7, and RPN11, whereas RPL17 did not yield detectable jackpot clones.

FIG. 26. Exemplary gels showing high GFP fluorescence clones (Jackpots) exhibit robust protein expression of AnxA3:GFP protein as measured by SDS-PAGE analysis, as well as high copy numbers for the integrated HapAmp construct as measured by dPCR.

FIG. 27. Schematic view of the co-transformation of the selectable-marker-less RPL25 HapAmp construct (linearized DNA) with the 2-micron episomal plasmid pBOND155, conferring antibiotic (KanMx) and/or auxotrophic (URA3) selection. This co-transformation yields RPL25 HapAmp jackpots for the expression of AnxA3.

FIG. 28. Schematic view of the co-transformation of the linearized marker-less RPL25 HapAmp construct with a 2-micron episomal plasmid conferring antibiotic selection (KanMx), alongside the expression of Cas9 and gRNA targeting double-strand breaks at a specific haploinsufficient locus (RPL25).

FIG. 29. Change in the essential amino acid profile for unengineered yeast (S3) and an engineered strain (S25), along with model prediction.

FIG. 30. Change in the amino acid profile for unengineered yeast (S3) and an engineered strain (S25), along with model prediction (predicted S25).

FIG. 31. Change in the amino acid profile for unengineered yeast (S3) and an engineered strain (S7), along with model prediction (predicted S7).

FIGS. 32A-32B. Analysis of total protein amino acid composition in strain S7 post-fermentation. FIG. 32A: Amino acid composition (% by weight) of wild-type (black bars), S7 (dark gray bars), and pure Ovis aries Annexin A3 (light gray bars). FIG. 32B: Concordance in amino acid content (individual amino acids represented by points) between pure O. aries Annexin A3 and the surplus amino acid content of strain S7. Concordance correlation coefficient (CCC) and identity line (dashed line) are shown. Amino acid composition of total strain protein was determined by total amino acid hydrolysis (AOAC 994.12, Alt. III, Midwest Laboratories), while Annexin A3 composition is based on its predicted sequence.

FIG. 33. Production stability of GgAnnexinA4 protein in post fermentation samples (n=38). S3 is the unengineered wild-type strain that does not express heterologous protein. i1-i10 represent ten, random post-fermentation isolates grown in production media, lysed and total protein run on SDS-PAGE and stained with Coomassie Blue.

FIG. 34. A graph presenting growth curves compared between a background yeast strain (S3) and an engineered yeast strain (S9).

FIG. 35. Target animal protein (as a % of Total Protein as measured by Mass Spec Total Protein Analysis) in strains expressing a single animal protein or a strain expressing 3 different proteins from the same species.

FIG. 36. Percent total protein as measured by the Kjeldahl method in unengineered yeast, strains expressing a single animal protein or a strain expressing 3 different proteins from the same species.

FIG. 37. Production stability of OaAnnexinA3 protein in post fermentation samples (n=53). S3 is the unengineered wild-type strain that does not express heterologous protein. i1-i10 represent ten, random post-fermentation isolates grown in production media, lysed and total protein run on SDS-PAGE and stained with Coomassie Blue.

FIG. 38. SDS-PAGE protein expression analysis of strains (S18, S25, and S36) expressing 2, 3, or 4 proteins, respectively, compared to an unengineered yeast (N-EY) strain. Protein expression of ANXA1, ANXA8, ANXA4, and FABP4 by mass spec is depicted in below the gel. MW=molecular weight marker.

FIGS. 39A-39C. SDS-PAGE gels of protein expression at different HapAmp integration sites. FIG. 39A shows ANXA5X2 integrated at RPL25, FIG. 39B shows ANXA4 integrated at RPL25 site with FABP5X2 integrated at RPL33, and FIG. 39C shows integration at three different sites; ANXA4 and FABP4 dual integration at RPL25, ANXA4 and ANXA1 dual integration at RPL33, and ANXA8 integrated at RPB7. MW=molecular weight marker

FIGS. 40A-40C. SDS-PAGE gels showing protein expression of annexins (FIG. 40A), fatty acid binding proteins (FIG. 40B), and retinol binding proteins (FIG. 40C) from red deer (RD) and water buffalo (WB). MW=molecular weight marker

FIG. 41. Chart comparing total protein content (by Kjeldahl) of the dried yeast expressing 1, 2, 3, or 4 different heterologous proteins. Total protein content increases as more proteins are integrated at different sites.

FIGS. 42A-42D. SDS-PAGE gels showing protein expression of FABP4 from red deer (RD) and FABP9 from water buffalo (WB) (FIG. 42A), FABP4 from RD and FABP12 from WB (FIG. 42B), FABP5 from RD (FIG. 42C), and FABP4 from WB (FIG. 42D).

### STATEMENTS REGARDING CHEMICAL COMPOUNDS AND NOMENCLATURE

In general, the terms and phrases used herein have their art-recognized meaning, which can be found by reference to standard texts, journal references and contexts known to those skilled in the art. The following definitions are provided to clarify their specific use in the context of the invention.

The following abbreviations are used herein: Anx or ANX refers to annexin or lipocortin; bp refers to base pair; CDS refers to coding sequence; FABP refers to fatty acid binding protein; GFP refers to green fluorescent protein; Gg or G. gallus refer to Gallus gallus; GOI refers to gene of interest; HapAmp refers to amplification of haploinsufficient genes; HARES refers to haploinsufficiency gene amplification rescue; HARESCO refers to haploinsufficiency gene amplification rescue co-transformation; KD or kDa refers to kilo Daltons; MS or Mass Spec refer to mass spectrometry; Oa or O. aries refer to Ovis aries. ORF refers to open reading frame; POI refers to protein of interest; RBP refers to retinol-binding protein; Sc or S. cerevisiae refer to Saccharomyces cerevisiae; WT refers to wild-type.

Throughout the present disclosure, efforts have been made to ensure accuracy with respect to font style used (e.g., italics, regular font, etc.) with respect to the terminology surrounding proteins and genes herein, but some error should be accounted for. One having skill in the art will appreciate the meaning of said terms based on the context in which they are used regardless of font style.

The term “about” is used herein to mean approximately, in the region of, roughly, or around. When the term “about” is used in conjunction with a numerical range, it modifies that range by extending the boundaries above and below the numerical values set forth. Unless otherwise stated, the term “about” is used herein to modify a numerical value above and below the stated value by a variance of 10%.

Unless the context clearly dictates otherwise, as used in the specification and the appended claims, the singular forms “a,” “an” and “the” include plural referents.

As used herein, “analog”, “analogous”, or “functionally analogous” refers to genes or proteins that have similar functions but did not evolve from a common ancestor. Instead, they evolved independently to have similar functions, a phenomenon known as convergent evolution.

As used herein “annexin-like protein” or “ALP” refers to a protein that can be robustly expressed in a heterologous host organism, particularly in a yeast and particularly in Saccharomyces and more particularly in Saccharomyces cerevisiae. In embodiments, the Annexin-like protein has one or more of the following structures or properties: the protein is an Annexin (also referred to in the art as lipocortin), an Annexin ortholog, an Annexin isoform or a fragment thereof, or has sequence identity of 30-60% of an Annexin, such as an Annexin of Table 1 or 2, and in particular has 30-60% sequence identity to an Annexin A1-A6, 8-10 or 11; or has positive net charge; or has molecular weight in the range of 20 to 75 KD, or more specifically 30 to 80 KD, or more specifically 30 to 75 KD or more specifically 40 to 70 KD, or more specifically 40-50 KD or any subranges thereof, or is a protein that is natively translated in the cytoplasm and then localized to the cell membrane; or is a protein that natively is not part of a protein complex; or a significant portion of the protein is alpha-helical in form, where the portion in alpha-helical form is 20% or more of the amino acid sequence, or more specifically 30% or more of the sequence, or more specifically 40% or more of the sequence, or more specifically 50% or more of the sequence or more specifically 60% or more of the sequence or more specifically 70% or more of the sequence. In some embodiments, the ALP has a sequence identity of 30-60% of an annexin, such as Annexin A7, A13, B1, B2, B3, B9, B10, B11, B12, B13, C1, D1, D2, D3, D4, D5, D6, D7, D8, or E1.

Unless otherwise specified, the term “average molecular weight” or “molecular weight” refers to number average molecular weight. Number average molecular weight is defined as the total weight of a sample volume divided by the number of molecules within the sample. As is customary and well known in the art, peak average molecular weight and weight average molecular weight may also be used to characterize the molecular weight of the distribution of polypeptides and/or proteins within a sample.

As used herein, “bind hydrophobic molecules” refers to the ability of a protein, in its functional, properly folded state, to interact with and form non-covalent associations with hydrophobic molecules, including but not limited to lipids, typically through hydrophobic regions or binding pockets. This binding occurs in an aqueous environment where the protein's hydrophobic-binding regions exhibit affinity for hydrophobic ligands. The scope of this definition encompasses proteins whether they are in their functional state or not, including proteins that are part of inactive or dormant organisms such as dried or desiccated fungi, or proteins that have undergone post-processing treatments such as drying, freezing, or other preservation methods. The capacity to bind hydrophobic molecules, including lipids, remains inherent to the protein structure, regardless of its current activation or processing state.

The term “engineered” or “recombinant” refers to a cell into which a recombinant gene, such as, for example, a gene encoding a heterologous protein, or part thereof, has been introduced. Therefore, engineered or recombinant cells are distinguishable from naturally occurring cells that do not contain a recombinant gene that is introduced by transfection, transformation, cell fusion, mating or other techniques. Recombinantly introduced genes will either be in the form of a cDNA (i.e., they will not contain introns), a copy of a cDNA gene, genomic DNA (with or without introns; for expression in prokaryotic hosts, the DNA should be without introns), or will include DNA sequences positioned next to a promoter not naturally associated with the particularly introduced gene.

As used herein, “enhanced expression” refers to an increase (e.g., at least 1%, at least 2%, at least 5%, at least 10%, or at least 25%) in the expression of a polypeptide of interest in a host organism.

As used herein “FABP-like protein” or “FLP” refers to a protein that can be robustly expressed in a heterologous host organism, particularly in a yeast and particularly in Saccharomyces and more particularly in Saccharomyces cerevisiae. In embodiments, the FABP-like protein has one or more of the following structures or properties: the protein is a FABP, a FABP ortholog, a FABP isoform or a fragment thereof; or has sequence identity of 30-60% of a FABP, such as a FABP of Table 11; or has positive net charge; or has molecular weight in the range of 20 to 75 KD, or more specifically 30 to 80 KD, or more specifically 30 to 75 KD or more specifically 40 to 70 KD, or more specifically 40-50 KD or any subranges thereof; or is a protein that is natively translated in the cytoplasm and then localized to the cell membrane; or is a protein that natively is not part of a protein complex; or a significant portion of the protein is alpha-helical in form, where the portion in alpha-helical form is 20% or more of the amino acid sequence, or more specifically 30% or more of the sequence, or more specifically 40% or more of the sequence, or more specifically 50% or more of the sequence or more specifically 60% or more of the sequence or more specifically 70% or more of the sequence. In some embodiments, the FLP has a sequence identity of 30-60% of a FABP, such as FABP1, FABP2, FABP3, FABP4, FABP5, FABP6, FABP7, FABP8, FABP9, FABP10, FABP11, or FABP12.

As used herein, “fusion protein” refers to a type of protein that is created by joining together two or more distinct protein domains or sequences from different genes or sources. The coding sequences of the selected protein domains are combined in a way that allows them to be expressed as a single protein within a cell. In some embodiments, one of the two or more distinct protein domains or sequences is a GFP.

As used herein, “fitness” refers to the ability of an organism to survive and reproduce in its environment. In some aspects, fitness refers to one or more of growth rate, viability, stress tolerance, competitive fitness, adaptability, or similar. In some embodiments, “fitness” refers to the “growth fitness” of an organism, which refers to an organism growth rate. In some aspects, this involves measuring how quickly an organism, such as yeast, can replicate and divide in a specific growth medium under controlled conditions. Organisms with faster growth rates are often considered more “fit”.

The term “gene amplification” refers to the increase in the number of gene copies. Gene amplification occurs naturally and can be induced in laboratory experiments (Deutschbauer et al., 2005 and Schimke, 1984). Peng et al., 2022, report a method that uses haploinsufficiency as an evolutionary force to drive in vivo gene amplification in S. cerevisiae using artificial genetic constructs based on haploinsufficient gene, this method is referred to as “HapAmp”. The reference reports gene amplification by tuning promoter strength or translational efficiency. The genetic constructs are described as incorporated into genetic vectors that can be used to introduce multiple copies of linked heterogeneous gene. Integration is reported to occur at the selected haploinsufficient locus in the yeast chromosome, in particular replacing the RPL25 promoter with the weaker BTS1 promoter. Deutschbauer et al., 2005 and Schimke, 1984 are each incorporated by reference herein in its entirety for descriptions of gene amplification, particularly in yeast. Peng et al., 2022 and any supplemental material associated with the reference is incorporated by reference herein in its entirety for descriptions and details of the method of gene amplification in Saccharomyces cerevisiae.

Methods and vectors herein rely in part of the haploinsufficient genes. Haploinsufficiency is the requirement for two wild-type copies of a gene for a normal phenotype. For haploinsufficient genes, when one copy of a gene is deleted or contains a loss-of-function mutation, the dosage of normal product generated by the single wild-type gene is not sufficient for complete function. As used herein, the term “deletion” in this context may refer to complete removal of the gene or a functional deletion of the gene such as by deleting only some portion of the gene so that a functional gene product is no longer expressed. This theory is referred to as the insufficient amount hypothesis (Cleary, 2001). A second theory, referred to as the balance hypothesis, predicts that the stoichiometry of various protein components is important for maintaining the integrity of a protein complex (Ohnuki & Ohya, 2018).

Deutschbauer et al., 2005, Delniri et al., 2008, Ohnuki & Ohya, 2018, Pir et al., 2012, Novo et al., 2013 and Shimada et al., 2013 are each incorporated by reference herein in its entirety for identification of haploinsufficient genes, particularly in yeast and particularly those haploinsufficient genes tied to cell fitness and/or cell growth. Ohnuki & Ohya, 2018, Pir et al., 2012 in particular identify haploinsufficient genes tied to cell growth which are useful in the methods described herein for heterologous gene amplification in yeast. Table 6 provides an exemplary list of haploinsufficient genes for use in the methods of this disclosure. The genomic location of a given (including those listed in Table 6) haploinsufficient genome is well known in the art and/or can be readily identified by one of ordinary skill in the art by reference to genome sequences in various publicly available databases, and/or can be readily determined by one in the art employing well known methods.

The term “heterologous” as used herein indicates molecules that are expressed in an organism other than the organism from which they originated or are found in nature. The molecule can have a coding region that is different from the host cell or a promoter region that is different from the host cell, or both. Alternatively, the terms “native” or “endogenous” as used herein indicates molecules that are expressed in the organism in which they originated or are found in nature. Endogenous nucleic acids and polypeptides can be heterologously expressed with expression levels lower, higher or otherwise alternatively regulated than the level of expression of the molecules in the native host cell. It is understood that expression of wild-type polynucleotides and polypeptides may be modified in recombinant host cells.

As used herein, “homologous,” “homology,” or “homolog” of a gene, a polypeptide, or fragments thereof, that shares a common evolutionary origin with another gene, polypeptide, or fragment in a different species. These homologous elements typically arise from a common ancestral genetic or structural element, and they may have undergone changes or diverged over time due to evolution. Homologs often serve similar functions in their respective species, although they may have adapted to different specific roles or environments. The term “homology.” “homologous,” or “homolog” also refer to a level of sequence similarity or sequence identity between two amino acid sequences (or among a set of such sequences) or between two nucleic acid sequences (or among a set of such sequences). In embodiments, homologous nucleic acid sequences are used to facilitate homologous recombination, for example into a host cell genome, the length of such homologous sequences and level of sequence homology or sequence identity of such sequences to the target sequences in the genome is that needed to accomplish homologous recombination. Methods of homologous recombination in various host organisms are well known in the art and one of ordinary skill in the art knows what length and what level of sequence homology or sequence identity is needed to facilitate such homologous recombination. For example, in embodiments where a promoter is homologous to at least a portion of the native promoter region of a haploinsufficient gene tied to yeast fitness, the promoter has sufficient has sufficient sequence identity to the native promoter to undergo homologous recombination with the native promoter. For example, in some embodiments, the homolog comprises a sequence having 50% or greater sequence identity to the native sequence. For example, in some embodiments, the homolog comprises a sequence having 50% or greater, 65% or greater, 75% or greater, 85% or greater, 95% or greater, or optionally 100% sequence identity to the native sequence.

As used herein, “hydrophobic molecule” refers to a molecule characterized by its low affinity for water and its tendency to repel aqueous environments. These molecules possess non-polar chemical structures, typically comprised of hydrocarbon chains or rings, that minimize interactions with polar solvents such as water. Hydrophobic molecules exhibit a propensity to aggregate in aqueous environments, often forming micelles or interacting with other hydrophobic substances. Examples of hydrophobic molecules include, but are not limited to, lipids, sterols, fatty acids, and certain hydrocarbons.

As used herein, “integrating” in the context of integrating a sequence into a host cell, refers to the process of stably incorporating a nucleic acid sequence, such as a genetic construct, into the genomic DNA of a host cell at a specific or non-specific location. This process results in the permanent inclusion of the genetic construct within the host genome, thereby creating a recombinant strain that expresses or modulates the expression of the introduced sequence in a manner that can be transmitted to progeny cells. In aspects, the integrating occurs through homologous recombination, or other mechanisms that result in stable incorporation into the host genome.

As used herein, “isoform” refers to a variant form of a protein, gene, or RNA molecule that is derived from the same gene or precursor molecule but differs in some way, often in its primary sequence or structure. Isoforms can arise due to alternative splicing of pre-mRNA during gene expression, post-translational modifications of proteins, or other mechanisms. These variants can have different functions, cellular localization, or activities, even though they originate from the same genetic or molecular source.

As used herein, “jackpot” or “modified HapAmp jackpot” refers to a transformed fungal strain, such as a transformed yeast strain, capable of enhanced expression of a heterologous polypeptide of interest, or a fusion protein thereof. In embodiments, a jackpot remains stably integrated at a selected haploinsufficient locus in the chromosome of the host. In embodiments, jackpots exhibit enhanced expression for at least 10 generations, such as for at least 10 generations, at least 20 generations, at least 50 generations, at least 100 generations, or at least 500 generations. In preferred embodiments, jackpots comprise a total heterologous polypeptide content, i.e., “a jackpot content”, of at least 2%, at least 5%, at least 10%, at least 15%, at least 20%, at least 25%, at least 40%, or at least 50%. “Jackpot efficiency” as discussed herein refers to the number of transformed fungi strains which are jackpots as compared to the total number of transformed fungi strains. In some embodiments, the jackpot efficiency is at least 2%, at least 5%, at least 10%, at least 15%, at least 20%, at least 25%, at least 40%, or at least 50% of the total protein content. In embodiments, jackpot content and jackpot efficiency depend on the identity of the heterologous polypeptide of interest, the gene copy number of the haploinsufficient gene, and/or the gene copy number of the heterologous polypeptide of interest. In some embodiments, the screening methods as described herein increase the jackpot content and/or the jackpot efficiency.

As used herein, “lipid” refers to one of a group of naturally occurring molecules that include fats, oils, waxes, and certain vitamins (e.g., A, D, E, and K). Lipids are hydrophobic or amphipathic, meaning they do not mix well with water but can interact with other fats and oils.

Fatty acids are a type of lipid that comprise long hydrocarbon chains with a carboxyl group (—COOH) at one end. Fatty acids are vital for various biological functions, including energy storage, cell membrane structure, and signaling. Some examples of fatty acids are palmitic acid, stearic acid, oleic acid, linoleic acid, and myristic acid. Other examples of lipids include triglycerides (fats and oils), phospholipids, sterols, and sphingolipids. Phospholipids are a type of lipid that are a major component of cell membranes. Examples of phospholipids include phosphatidylcholine, phosphatidylserine, phosphatidylethanolamine, phosphatidylinositol, phosphatidylglycerol, cardiolipin, phosphatidic acid, and lyso-phosphatidylcholine. Examples of sterols include ergosterol, lanosterol, zymosterol, and episterol.

Sphingolipids include ceramides, inositol phosphorylceramide, mannosylinositol phosphorylceramide, and mannsyl-diinositol phosphorylceramide. There are also neutral lipids, which include triacylglycerols, diacylglycerol, and monoacylglycerol. Lipid signaling molecules include inositol-1,4,5-triphosphate, diacylglycerol, and sphingosine-1-phosphate. Glycolipids, like glycosylphosphatidylinositol, are another type of lipid.

There are also lipid droplets which are storage organelles in cells that primarily contain triacylglycerols and steryl esters. Furthermore, fungus cells also contain steryl esters (e.g., ergosteryl esters), polyisoprenoids (e.g., dolichol and undecaprenyl phosphate), lipid peroxides, glycosphingolipids, phosphoinositides, lipid-linked proteins, fatty acyl-CoA esters (e.g., acyl-CoA derivatives), plasmalogens, fatty alcohols, and wax esters.

As used herein, “loss-of-function mutation” refers to a type of genetic mutation that results in the reduction or complete loss of the normal function of a gene or protein. These mutations typically disrupt the structure or function of the gene or protein product, leading to a decrease or elimination of its biological activity. In some embodiments, a loss-of-function mutation comprises the total removal of the gene.

As used herein, “operably linked” or “in operable linkage” refers to a functional linkage between two nucleic acid sequences, such a control sequence (typically a promoter) and the linked sequence (typically a sequence that encodes a protein, also called a coding sequence). A promoter is in operable linkage with an exogenous gene if it can mediate transcription of the gene. A coding sequence is also inoperable linkage with a terminator sequence if the terminator sequence mediates the termination of transcription. Nucleic acid sequences can be considered “in operable linkage” with each other as long as they are structurally linked in an order that facilitates transcription and/or termination of transcription, even if the nucleic acid sequences are inside a cell that is no longer living or viable (such as in instances where the biological material, including fungi or other cells, is in a dormant or inactive state, such as in dried or desiccated form).

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, “orthologs” and “ortholog” refer to genes or proteins which may be homologs, as defined herein. In some embodiments, an ortholog diverged through a speciation event. In other words, orthologs arose from a common ancestor gene in the last common ancestor of two different species. Orthologs often retain similar functions and are found in different species. In some embodiments, the ortholog comprises a sequence having 50% or greater sequence identity to the native sequence. For example, in some embodiments, the ortholog comprises a sequence having 50% or greater, 65% or greater, 75% or greater, 85% or greater, 95% or greater, or optionally 100% sequence identity to the native sequence

A “peptide” or “polypeptide” herein are used interchangeably and refer to a polymer of repeating structural units connected by a peptide bond. Typically, the repeating structural units of the peptide are amino acids, including naturally occurring amino acids, and analogues of amino acids or any combination of these. The number of repeating structural units of a peptide, as understood in the art, are typically less than a “protein”, and thus the peptide often has a lower molecular weight than a protein. As is well understood in the art, a protein contains one or more polypeptides. Thus, in some embodiments described herein, the polypeptide refers to a protein.

The term “percent identity”, in the context of two or more nucleic acid or polypeptide sequences, refers to a specified percentage of nucleotides or amino acid residues that are identical as between or as among the sequences when aligned for maximum correspondence. Optimal alignment of sequences for comparison can be conducted by various art-recognized methods, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, PASTA, and TFASTA in the Wisconsin Genetics Software Package, Genetics Computer Group, 575 Science Dr., Madison, Wis.), or by visual inspection (see generally, Ausubel F M, et al., editors. Current Protocols in Molecular Biology. Chapters 1-6. Vol. 1. John Wiley & Sons, Inc.; Hoboken, N.J., United States of America: 2008).

Unless otherwise specified, “percent identity” is assessed herein using the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/), and unless otherwise specified, “percent identity” is measured using BLASTP or BLAS TN with default parameters at (www.ncbi.nlm.nih.gov). Depending on the application, the percent “identity” can exist over a region (e.g. a fragment) of the sequence being compared, e.g., over a functional domain, or, alternatively, exist over the full length of the two sequences to be compared.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

As used herein, “POT1” refers to the plasmid system in S. cerevisiae which uses S. pombe POT1 as a selectable marker for maintenance of the plasmid. In some embodiments, POT1 refers to the triose phosphate isomerase gene from the fission yeast Schizosaccharomyces pombe, as provided in Kawasaki et al. ZYMOGENETICS Inc. 1990 Jun. 5. Stable DNA constructs for expression of α-1 antitrypsin, which is hereby incorporated by reference for information related to POT1 and uses thereof.

As used herein, “pink” refers to a color which an individual having normal vision would perceive as pink, light red, or salmon.

The term “recombinant” as used herein, for example, with respect to nucleic acid, protein/polypeptide/peptide, or host cell, refers to the preparation of hybrid nucleic acid molecules which comprise at least two nucleic acid fragments that do not usually occur together in nature and to generation of protein/polypeptide/peptide from such hybrid nucleic acid molecules in the host cell. Recombinant processes can include, among others, the combination (e.g., ligation) of nucleic acids from different sources (e.g., combining a nucleic acid coding sequence with one or more nucleic acid regulatory sequences heterologous to the coding sequence, or insertion of such an expression cassette into an expression or other vector or plasmid in which the expression cassette is not naturally found), insertion of a nucleic acid coding sequence (e.g., encoding a protein sequence) into a heterologous host. Recombinant expression is expression of a heterologous nucleic acid and/or protein/polypeptide/peptide in a host organism. The term “recombinant host cell” as used herein refers in most instances to a host cell(s) that has been genetically modified to express heterologous polynucleotides or polypeptides, such as those included in an expression vector, or in an integration construct. Recombinant expression also refers to a host cell that has been genetically modified to modify expression of endogenous polynucleotides, for example, to alter regulatory control of expression such that expression is altered from that in the endogenous (e.g., natural) host (e.g., overexpression, tissue specific expression and the like). “Altering” or an “alteration” in expression refers to expression of the gene, or level of a RNA molecule or equivalent RNA molecules encoding one or more polypeptides or polypeptide subunits, or activity of one or more polypeptides or polypeptide subunits is up regulated or down regulated, such that expression, level, or activity is greater than or less than that observed in the absence of the alteration, e.g., in the endogenous expression environment, e.g., endogenous host. For example, the term “alter” can include inhibit or enhance or regulate gene expression in a way that is different from that in the endogenous host. A recombinant protein/polypeptide/peptide is used herein to refer to a protein/polypeptide/peptide that is produced by a recombinant process or technology, for example, by inserting a heterologous gene or expression cassette into a host cell to have the host cell produce the heterologous amino acid sequence, peptide, protein or fragment thereof.

As used herein, the term “region” refers to a specific, continuous segment of a gene or genetic construct that performs a particular function or contains specific elements contributing to the overall activity or regulation of the gene or construct. For example, a “promoter region” is used interchangeably with the term “promoter” and refers to the sequence upstream of the transcription start site that contains elements necessary for the initiation of transcription. For example, a “terminator region” is used interchangeably with the term “terminator” and refers to the sequence downstream of a coding sequence that signal the end of transcription. For example, a “tandem amplification region” refers to a segment of DNA containing multiple, consecutive copies of a particular sequence or gene arranged in series.

As used herein, the term “sequence homology” or “sequence identity”, with respect to peptides, means the proportion of amino acid matches between two amino acid sequences of interest in two different peptides considering the ordering of the amino acids. Matches occur when amino acids are in the same order in one peptide compared to the other peptide. With respect to genes, sequence homology or sequence identity means the proportion of nucleic acid matches between two nucleic acid sequences of interest in two different genes considering the order of the nucleic acids. Matches occur when amino acids or nucleic acids are in the same order in one peptide or one gene compared to the other peptide or other gene, respectively. When sequence homology is expressed as a percentage, e.g., 50%, the percentage denotes the fraction of matches over the length of sequence that is compared to some other sequence, considering the amino acid or nucleic acid order. With respect to genes, gaps (in either of the two sequences) are permitted to maximize matching; for example, in genes wherein gap lengths of 5 nucleic acids or less, optionally 3 nucleic acids or less, are usually used. In other words, a sequence having 75% or greater sequence identity to a nucleic acid sequence with 9 nucleic acids can indicate that the 9 nucleic acid sequence can have one or two point mutations (i.e., nucleic acid change), one or two nucleic acid deletions, one or two nucleic acid additions, one point mutation and one nucleic acid deletion, or one point mutation and one nucleic acid addition, provided that such nucleic acid change does not result in a frameshift mutation. Even with two such nucleic acids being different, 7 out of 9 nucleic acids still match in the correct order, such that there is greater than 75% sequence identity. For clarity, the analysis of whether there is sequence homology between two nucleic acid sequences of interest is conducted with respect to a particular portion of one gene (i.e., a first nucleic acid sequence of interest) relative to a particular portion of another gene (i.e., a second nucleic acid sequence of interest), and is not conducted relative to all nucleic acids present in a gene (i.e., the analysis does not include nucleic acids outside of the particular nucleic acid sequence of interest). With respect to peptides or proteins, gaps (in either of the two sequences) are permitted to maximize matching; for example, wherein gap lengths of 5 amino acids or less, optionally 3 amino acids or less, are usually used. In other words, a sequence having 75% or greater sequence identity to an amino acid sequence with 9 amino acids can indicate that the 9 amino acid sequence can have one or two point mutations (i.e., amino acid change), one or two amino acid deletions, one or two amino acid additions, one point mutation and one amino acid deletion, or one point mutation and one amino acid addition. Even with two such amino acids being different, 7 out of 9 amino acids still match in the correct order, such that there is greater than 75% sequence identity. For clarity, the analysis of whether there is sequence homology between two amino acid sequences of interest is conducted with respect to a particular portion of one peptide or protein (i.e., a first amino acid sequence of interest) relative to a particular portion of another peptide or protein (i.e., a second amino acid sequence of interest), and is not conducted relative to all amino acids present in a peptide or protein (i.e., the analysis does not include amino acids outside of the particular amino acid sequence of interest).

The term “sufficient amount” means an amount sufficient to produce a desired effect, e.g., an amount sufficient to modulate protein aggregation in a cell.

The term “substantially free” refers to a composition that comprises a desired compound, desired compounds, and optional inert compounds and is free of significant quantities of an undesired compound or undesired compounds. A typical substantially free composition comprises greater than about 80% by weight of the desired compound, desired compounds, and optional inert compounds and less than about 20% by weight of one or more other undesired compounds, more preferably greater than about 90% by weight of the desired compound, desired compounds, and inert compounds and less than about 10% by weight of one or more other undesired compounds, even more preferably greater than about 95% by weight of the desired compound, desired compounds, and inert compounds and less than about 5% by weight of one or more other undesired compounds, and most preferably greater than about 97% by weight of the desired compound, desired compounds, and inert compounds and less than about 3% by weight of one or more other undesired compounds.

As used herein, “synthetic open reading frame” or “synthetic ORF” refers to a DNA sequence which is not naturally found at a specific locus of a fungal genome. In some embodiments, the synthetic ORF is derived from the same species of the transformed fungi strain. In other embodiments, the synthetic ORF is derived from a different species of the transformed fungi strain. In some embodiments, the synthetic ORF comprises a naturally occurring sequence. In some embodiments, the synthetic ORF comprises a naturally occurring sequence which has been genetically modified. In some embodiments, the synthetic ORF comprises an artificial construction of a natural or non-natural sequence.

As used herein, “tied to” in the context of a gene “tied to cell fitness” and/or “tied to cell growth” and/or “tied to fungi fitness” refers to a gene that has a direct or indirect influence on the viability, proliferation, survival, or overall health of a cell. This influence can be established through experimental data, genetic correlation, or observed phenotypic effects where the expression, regulation, or modification of the gene affects cellular functions related to fitness. The term encompasses genes whose activity, expression level, or mutation status contributes to, or is associated with, the fitness characteristics of the cell under specific conditions.

As used herein “total protein content” or “total polypeptide content” refers to crude protein content wherein all proteins and polypeptides, regardless of identity or function, are included in the measurement. Crude protein is typically measured by e.g. the Kjeldahl method or a modified Kjeldahl method which is based on nitrogen content. Protein content can also be measured by Bradford or BCA/copper sulfate protein assays. Bradford quantifies the total protein content by detecting the presence of proteins based on their ability to bind to a dye called Coomassie Brilliant Blue G-250. BCA/CuSO4 quantifies the total protein content by detecting the presence of proteins and their ability to reduce the BCA reagent in the presence of copper ions. In some embodiments, total protein content and/or total polypeptide content is measured by tandem MS, by such methods that are well known in the art, this method measures protein that is detectable by MS. The protein content can also be measured by a combustion method, which is based on nitrogen content and known in the art.

The term “transformation” refers to the transfer of a nucleic acid fragment into a host organism, resulting in genetic inheritance. Genetic inheritance can be stable or unstable. Host cells (e.g., eukaryotic cells) containing the transformed nucleic acid fragments are referred to as “transgenic” or “recombinant” or “transformed”. In an embodiment, transformation involves integration of a heterologous DNA molecule into the genome of a host organism. In embodiments, introducing exogenous nucleic acid into a host cell generates expression products of the nucleic acid to affect a measurable change in one or more properties of the host cell. For example, a mutation in the host cell may be corrected/rescued by such transformation. For example, antibiotic resistance may be conferred on an antibiotic sensitive host cell by such transformation. For example, a transformed host cell may express a heterologous protein. In embodiments, transformation involves integration of exogenous nucleic acid, particularly expressible exogenous nucleic acid, into the genome of the host. In embodiments, transformation can include integration of one or more copies of introduced exogenous nucleic acid into the genome of the host organism.

As used herein, “weaker” and “stronger” in the context of promoters refer to the relative transcriptional activity of different promoter sequences. A “weaker” promoter drives lower levels of transcription and gene expression compared to a “stronger” promoter under similar conditions. In aspects, this difference in activity is due to variations in the promoter's sequence that impact its binding affinity for transcription factors or RNA polymerase. In aspects, this difference in activity is due to the presence of regulatory elements that either inhibit or enhance transcriptional initiation or elongation. These terms describe the comparative capability of promoters to regulate the expression of an operably linked gene. In aspects, the terms “weaker” and “stronger” are also used in comparison to the native promoter of a gene, where a “weaker” promoter produces lower levels of gene expression and a “stronger” promoter produces higher levels of gene expression relative to the naturally occurring (native) promoter under the same conditions.

As used herein, the terms “wild-type” and “unengineered” are used interchangeably to refer to a naturally occurring cell that has not been genetically modified or engineered. Specifically, a wild-type cell and an unengineered cell do not contain a recombinant gene that is introduced by transfection, transformation, cell fusion, mating or other techniques. More specifically, a wild-type cell and an unengineered cell have not had a gene encoding a heterologous protein, or part thereof, introduced to its genome.

In an embodiment, a composition or compound of the invention, such as an alloy or precursor to an alloy, is isolated or substantially purified. In an embodiment, an isolated or purified compound is at least partially isolated or substantially purified as would be understood in the art. In an embodiment, a substantially purified composition, compound or formulation of the invention has a chemical purity of 95%, optionally for some applications 99%, optionally for some applications 99.9%, optionally for some applications 99.99%, and optionally for some applications 99.999% pure.

### DETAILED DESCRIPTION OF THE INVENTION

In the following description, numerous specific details of the compositions, composition components and methods of the present invention are set forth in order to provide a thorough explanation of the precise nature of the invention. It will be apparent, however, to those of skill in the art that the invention can be practiced without these specific details.

In one aspect, the disclosure relates to the production of heterologous proteins in transformed yeast and particularly to production of enhanced levels of heterologous protein in transformed yeast and more particularly to production of enhanced levels of heterologous protein in transformed yeast wherein the growth fitness of the yeast producing the enhanced levels of heterologous protein is not significantly decreased compared to untransformed yeast. In aspects, transformed yeast exhibit levels of heterologous protein of 15% or more of total protein produced by the transformed yeast. In aspects, transformed yeast, exhibit growth rates within 40% or less of that of untransformed yeast. In aspects, transformed yeast exhibit levels of heterologous protein of 10% or more of total protein produced by the transformed yeast. In aspects, transformed yeast, exhibit growth rates within 30% or less of that of untransformed yeast. More specifically, transformed yeast exhibit levels of heterologous protein of 5% or more of total protein produced by the transformed yeast. More specifically, transformed yeast, exhibit growth rates within 20% or less of that of untransformed yeast. Yet more specifically, transformed yeast expressing heterologous protein exhibit levels of heterologous protein of 5% or more of total protein produced and exhibit growth rates within 15% or less of that of untransformed yeast. Heterologous protein produced in such transformed yeast can be employed for any purpose. In embodiments, enhanced heterologous protein production can be used to generate pharmaceutical proteins, industrial proteins or nutritional proteins.

In a related aspect, the disclosure relates to the enhanced production of heterologous nutritional protein in yeast and more specifically to the enhanced production of annexins, FABPs, and retinoid binding proteins in yeasts. Annexins, FABPs, and retinoid binding proteins heterologously produced in yeast are useful, for example, as protein in food compositions. One or more annexin coding sequences can be transformed into yeast by any known method such that the annexin-transformed yeast exhibit levels of heterologous protein of 1% (more preferably 2% and yet more preferably 5%) or more of total protein produced by the transformed yeast. In aspects, annexin-transformed yeast, exhibit growth rates within 40% or less of that of untransformed yeast. More specifically, annexin-transformed yeast, exhibit growth rates within 30% or less of that of untransformed yeast. More specifically, annexin-transformed yeast, exhibit growth rates within 20% or less of that of untransformed yeast. More specifically, annexin-transformed yeast, exhibit growth rates within 15% or less of that of untransformed yeast. In aspects, FABP-transformed yeast, exhibit growth rates within 40% or less of that of untransformed yeast. More specifically, FABP-transformed yeast, exhibit growth rates within 30% or less of that of untransformed yeast. More specifically, FABP-transformed yeast, exhibit growth rates within 20% or less of that of untransformed yeast. More specifically, FABP-transformed yeast, exhibit growth rates within 15% or less of that of untransformed yeast. In aspects, retinoid binding protein-transformed yeast, exhibit growth rates within 40% or less of that of untransformed yeast. More specifically, retinoid binding protein-transformed yeast, exhibit growth rates within 30% or less of that of untransformed yeast. More specifically, retinoid binding protein-transformed yeast, exhibit growth rates within 20% or less of that of untransformed yeast. More specifically, retinoid binding protein-transformed yeast, exhibit growth rates within 15% or less of that of untransformed yeast. Yet more specifically, transformed yeast expressing heterologous protein exhibit levels of heterologous protein of 5% or more of total protein produced and exhibit growth rates within 40% or less of that of untransformed yeast. Yet more specifically, transformed yeast expressing heterologous protein exhibit levels of heterologous protein of 5% or more of total protein produced and exhibit growth rates within 30% or less of that of untransformed yeast. Yet more specifically, transformed yeast expressing heterologous protein exhibit levels of heterologous protein of 5% or more of total protein produced and exhibit growth rates within 20% or less of that of untransformed yeast. Yet more specifically, transformed yeast expressing heterologous protein exhibit levels of heterologous protein of 5% or more of total protein produced and exhibit growth rates within 15% or less of that of untransformed yeast.

In other related aspects, the disclosure relates to methods for increasing the copy number of expressible coding sequences of heterologous proteins in transformed yeast and thereby enhancing the production of the heterologous protein in the transformed yeast. Such methods can generate transformed yeast which exhibit levels of heterologous protein of 5% or more of total protein produced by the transformed yeast. Such methods can generate transformed yeast, exhibiting growth rates within 40% or less of that of untransformed yeast.

Heterologous proteins, the expression of which can be enhanced by the methods herein, include recombinant therapeutic protein and peptide products (e.g., human biologics, insulin and related protein therapeutics), industrial protein products (e.g., enzymes) and nutritional proteins (e.g., animal proteins), among others. Therapeutic proteins/polypeptides or peptides are intended for use in the treatment of certain disease conditions in humans and non-human animals. In embodiments, nutritional proteins are employed to provide protein in various food products for human and animal consumption. Nutritional proteins are generally non-toxic to the human or animal for whom/which the food product is intended. In embodiments, nutritional proteins are animal proteins. Heterologous proteins used in food composition need not retain their native function. The heterologous protein is also called the protein of interest. The expression cassette or gene comprising the heterologous protein in which the coding sequence of the heterologous protein is operably linked is also termed the gene of interest.

More specifically, therapeutic proteins/polypeptides and peptides include, among others, human serum albumin, insulin and related peptide products (e.g., glucagon-like peptide 2), proteins/polypeptides/peptides of the immunoglobulin superfamily (e.g., antibodies, T-cell receptors, MHC, cell surface receptors involved in the immune response), adhesion receptors and cell signaling proteins/polypeptides/peptides, including, e.g., cytokines such as interleukins, interferons (e.g., IFNalpha2beta, human interferon beta). Heterologous therapeutic proteins/polypeptides/peptides useful in methods herein include those that have been or are currently produced in any yeast expression system, including insulin (e.g., human insulin), IFNalpha2beta, hepatitis B antigen, glucagon like peptide 2, human interferon beta, human granulocyte-macrophage colony-stimulating factor, human serum albumin, hirudin, human transferrin, glucagon, hepatitis surface antigen, platelet-derived growth factor, granulocyte colony stimulating factor, urate oxidase, rotovirus VP6 protein, HPV type 16 L1-L2 chimeric protein. In embodiments, recombinant therapeutic proteins include monoclonal antibodies drugs, hormones and vaccines.

Recombinantly produced therapeutic proteins/polypeptides/peptides retain some measurable level of therapeutic activity compared to the analogous therapeutic proteins/polypeptides/peptides from its natural source. Recombinant therapeutic proteins/polypeptides/peptides preferably retain a minimum of 10% of the activity of the natural analog and more preferably retain at least 50% of the activity of the natural analog.

Heterologous proteins include enzymes which are useful as catalysts in various commercial applications in the food/feed/dairy/baking industry, in the detergent/cleaner industry, and the textile industry. Exemplary enzymes that can be recombinantly produced include among others, amylase (e.g., alpha-amylase), glycoamylase, glucanase, laccase, pepsin, chymosin, phytase, protease, lipase, cellulase, xylanase, mannanase, and pectate lyases. Heterologous enzymes useful in methods herein include those that have been or are currently produced in any yeast expression system. Recombinantly produced enzymes retain some measurable level of catalytic activity compared to the analogous enzyme from its natural source. Recombinant enzymes preferably retain a minimum of 10% of the activity of the natural enzyme analog and more preferably retain at least 50% of the activity of the natural enzyme analog.

Heterologous proteins/polypeptides/peptides include various proteins/polypeptides and peptides useful for inclusion into food for primarily humans and animals, particularly pets (e.g., companion animals). Any recombinant animal protein can be used with the methods and compositions of the disclosure. The recombinant animal protein used with the methods and compositions of the disclosure may be a full-length protein, a truncated protein, or a fragment of a protein. A fragment (or portion of a protein) is an amino acid sequence that has at least three amino acids of the full-length protein. In some embodiments, the full-length protein is produced by expressing fragments that cover the full-length protein.

In some embodiments, the heterologous animal protein has a higher percentage of essential amino acids compared to other animal tissue proteins. In some embodiments, the heterologous animal protein comprises more than 1%, 2%, 3%, 4%, 5%, 6%, 7%, 8%, 9%, 10%, 11%, 12%, 13%, 14%, 15%, 20%, 25%, 30%, 31%, 32%, 33%, 34%, 35%, 36%, 37%, 38%, 39% or 40% of essential amino acids of the animal for which a food comprising the animal protein is intended. The essential amino acids in humans and non-human animals are histidine, isoleucine, leucine, lysine, methionine, phenylalanine, threonine, tryptophan and valine. Arginine is an additional essential amino acid in a number of non-human animals (e.g., birds, carnivores including dogs and cats and certain farm animals (e.g., cows, pigs)).

Non-limiting examples of animal proteins that can be used as heterologous proteins with the methods and food compositions of the disclosure are, among others: troponin I, actin, myosin, destrin, alpha-actinin-2, alpha-actinin-3, titin, receptor tyrosine protein, kinase skeletal muscle, myosin binding protein C, F-actin-capping protein, Myosin-binding protein H, troponin T, myotubularin 1, myozenin-1, beta-enolase, cofilin-2, PDZ and LIN1 domain protein 7, twinfilin-2, telethonin, M-protein striated muscle, coronin, nebulin-relatedanchoring protein, myopalladin, tensin, gelsolin, dystroglycan, profilin, myozenin-2, calsarcin 1, myotilin, paxillin, integrin alpha-7, integrin beta-I, dystrophin, ankyrin, paranemin, myomesin (skelemin), alpha sarcoglycan, gamma sarcoglycan, or calponin.

In embodiments, animal proteins include animal muscle proteins including skeletal muscle proteins and cardiac or smooth muscle proteins. Non-limiting examples of animal muscle proteins (or relatives of those proteins) that can be used with the methods and food compositions of the disclosure include: thymosin beta 4, metavinculin, parvalbumin beta, tripartite motif-containing protein 54, obscurin, muscle M-line assembly protein unc-89, muscle-type aldolase, SERCA1, calponin homology-associated smooth muscle protein, skeletal muscle ankyrin repeat protein, calpain-3, atrogin-1, striated muscle-specific serine/threonine-protein kinase, skeletal muscle LIJVI-protein 2, glycogen phosphorylase, serpin A3-1, cadherin, beta-taxilin, density-regulated protein, synaptopodin, ARP2/3, WASP, SCAR/WA VE, IQGAP, AbpI, cortactin, drebrin, ENAN ASP, annexin II, BPAG, ERM protein, Sla2, utrophin, Srv2/CAP, verprolin, formins, capZ, fragmin, villin, AIP1, adducin, MACF, J\tIAP2, tau, fimbrin, scruin, espin, fascin, actinfilin, actinogelin, Ark 1, Prkl, actobindin, actolinkin, alpha-parvin, actophorin, acumentin, scinderin, afadin, AFAP-110, affixin, aginactin, angiogenin, dystonin, anilin, archvillin, cortactin, caltropin, CARMIL, caerin-1.16, dematin, diaphanous, EF1-alpha, EF1-beta, LIM domain and actin-binding protein, elongation factor 2, epsin, proheparin-binding EGF-like growth factor, Mitogen-activated protein kinase, frabin, four and a half LIM domains protein 3, FH1/FH2 domain-containing protein 3, GAS2-like protein 2, kettin, Kelch protein, limatin, PDZ and LIM domain protein 1, synaptopodin-2, prefoldin, presenilin I, receptor tyrosine-protein kinase erbB-2, protein kinase C, striated muscle-specific serine/threonine-protein kinase, rapsyn, shroom, smitin, smoothelin, or serine/threonine-protein phosphatase, laminin, sarcospan, dystrobrevin, syntrophin, dysbindin, dysferlin, or fukutin.

Preferred animal protein sequences useful in the disclosure are listed in Table 1 of published International patent application wo2020/160187, published Aug. 6, 2020. This published patent application is incorporated by reference herein in its entirety for among others, descriptions of animal proteins and food composition containing such proteins. In this table animal proteins are grouped according to the tissue in which they are highly expressed (if known). If it is not known in what tissue a protein is expressed, the protein is grouped according to the tissue for which its expression is required (e.g., for normal development of the tissue). For example, it is known that myotubularin is required for normal skeletal muscle growth. Thus, it is grouped with the skeletal muscle proteins. Persons skilled in the art will appreciate that in some cases a protein can be expressed in more than one tissue types.

In some embodiments, the animal protein is an actin cytoskeleton protein. In some embodiments, the actin cytoskeleton protein is a filament protein, a capping protein, an actin binding protein, an actin-bundling protein, a monomer binding protein, a cytoskeletal linker protein, a membrane anchor protein, a stabilizing protein, a signaling protein, a capping protein, a severing protein, or a myosin.

In specific embodiments, the heterologous protein/animal protein is an annexin, also referred to in the art as lipocortin. Annexins are a class of calcium-dependent membrane-binding proteins that associate with different components of the cytoskeleton and mediate protein interactions between the cell and the extracellular matrix. Twelve different genes have been identified in humans, Annexin A1-A11, and A13. Annexins are not found in yeasts and prokaryotes. Certain annexins have been found to be particularly useful for overexpession in yeast without interfering with native yeast proteins. Annexins have low sequence identity and similarity to yeast endogenous proteins, high solubility, medium size, acidic isoelectric point and are rich in alpha-helical secondary structure. One of or a combination of these properties make annexins particularly useful for expression in a heterologous host, such as yeast.

Exemplary Annexins useful in the present disclosure are listed in Table 1 herein. Table 1 lists the name of the annexin and the database designation number of the protein/gene. In embodiments, the Annexin is Annexin A1-A11 or Annexin A13 or isoforms or fragments thereof. In embodiments, the Annexin is Annexin B1-B3, Annexin B9-B13, Annexin C1, Annexin D1-D8 or Annexin E1, or isoforms or fragments thereof. In embodiments, the Annexin is Annexin A1, Annexin A5, Annexin A4, Annexin A2 or Annexin A13. In embodiments, the Annexin is other than Annexin A6. In embodiments, the Annexin is other than Annexin A2. In embodiments, the Annexin is an Annexin listed in Table 1. In embodiments, the Annexin is an Annexin from any animal source. In embodiments, the Annexin is an Annexin from any mammalian source. In embodiments, the Annexin is an Annexin from a non-human animal source. In embodiments, the Annexin is an Annexin from a bird source. In embodiments, the Annexin is an Annexin from a fish source. In embodiments, the Annexin is from chicken, duck, turkey, pig, sheep, cow, rabbit, deer, red deer, buffalo, water buffalo, salmon, cod, tilapia or tuna. In embodiments, the Annexin is Annexin A1-A4 or Annexin A6 isoform X1 of lamb (SEQ ID NO. 13; SEQ ID NO. 68; SEQ ID NO. 82; SEQ ID NO: 352; or SEQ ID NO: 121, respectively). In embodiments, the Annexin is Annexin A1 or a fragment thereof, Annexin A2, Annexin A4, Annexin A5 or Annexin A13 isoform X1 of chicken (SEQ ID NO. 65; SEQ ID NO. 88; SEQ ID NO. 102; or SEQ ID NO: 47, respectively). In embodiment, the Annexin is Annexin A2 of cow (SEQ ID NO. 63).

In some embodiments, the heterologous protein is an Annexin-like protein. In embodiments, annexin-like proteins exhibit robust or enhanced expression in host organisms, particularly in yeast and more particularly in Saccharomyces cerevisiae. For example, robust or enhanced expression is such that a heterologous protein is expressed in the heterologous host at a level of at least 2% of total protein. If more than one heterologous protein is expressed, robust or enhanced expression is such that the total expression level of the mixture of proteins is at a level of at least 2% of total protein. In embodiments, heterologous protein expression ranges from 2-50% of total protein of the host organism, including all subranges thereof. In embodiments, total heterologous protein expression ranges from 2.5% to 25% of total protein. In embodiments, total heterologous protein expression ranges from 2.5% to 50% of total protein. In embodiments, total heterologous protein expression ranges from 2.5% to 35% of total protein. In embodiments, total heterologous protein expression ranges from 2.5% to 30% of total protein. In embodiments, total heterologous protein expression ranges from 3% to 30% of total protein. In embodiments, total heterologous protein expression ranges from 4% to 30% of total protein. In embodiments, total heterologous protein expression ranges from 5% to 50% of total protein. In embodiments, total heterologous protein expression ranges from 5% to 30% of total protein. In embodiments, total heterologous protein expression ranges from 5% to 25% of total protein. In embodiments, total heterologous protein expression is greater than 2%, or 3%, or 4% or 5% or 6% or 7% or 8% or 9% or 10% or 12% or 15% or 18% or 20% or 25% or 30% or 40% or 45% of total protein.

In embodiments, the heterologous protein is a protein having 85% (or 86%, 87%, 88%, 89%, 90%, 91%, 92%, 93%, 94%, 95%, 96%, 97%, 98%, 99%) or higher amino acid sequence identity to an Annexin of Table 1 and Table 2. In embodiments, the heterologous protein is a protein having an Annexin sequence of Table 1 and Table 2 with 1, 2, 3, 4, 5, 6, 7, 8, 9 or 10 conservative amino acid substitutions. In embodiments, the heterologous protein/polypeptide is a fragment of an Annexin of Table 1 or Table 2. In embodiments, a fragment of an Annexin contains between 20-95% of the amino acid sequence of a full-length Annexin including any subranges thereof and further including fragments containing 25%, 30%, 35%, 40%, 45%, 50%, 55%, 60%, 65%, 70%, 75%, 80%, 85%, 90%, 95% of the amino acid sequence of a full-length Annexin. Annexin fragments can be generated by deleting or excluding a C-terminal region, an N-terminal region or an internal region of the amino acid sequence of a known Annexin, such as an Annexin of Table 1 or Table 2.

TABLE 1
Exemplary Annexins.

| Annexin | Accession # | Animal | Annexin | Accession # | Animal |
|---|---|---|---|---|---|
| Annexin A13 | A0A1D5NUB1 | Gallus gallus | Annexin A2 | P04272 | Bos Taurus |
| Annexin A1 | Q92108 | Gallus gallus | Annexin A1 | NP 786978.2 | Bos Taurus |
| (fragment) | | | | | |
| Annexin A6 | P51901 | Gallus gallus | Annexin A3 | NP 001030402.1 | Bos Taurus |
| Annexin A5 | FINJIO | Gallus gallus | Annexin A4 | NP 001001440.2 | Bos Taurus |
| Annexin A4 | XP_040545559.1 | Gallus gallus | Annexin A5 | NP 001035567.3 | Bos Taurus |
| Annexin A2 | P17785 | Gallus gallus | Annexin A6 | NP 001096694.1 | Bos Taurus |
| Annexin A11 | XP_046775996.1 | Gallus gallus | Annexin A7 | NP_001069459.1 | Bos Taurus |
| isoform X1 | | | | | |
| Annexin A1 | NP_996789.2 | Gallus gallus | Annexin A8 | NP 776666.1 | Bos Taurus |
| Annexin A7 | NP_001264276 | Gallus gallus | Annexin A9 | NP 001030450.1 | Bos Taurus |
| Annexin A8 | XP_040531195.1 | Gallus gallus | Annexin A10 | NP 001178987.1 | Bos Taurus |
| Annexin A10 | XP_040527760.1 | Gallus gallus | Annexin A11 | NP 776927.1 | Bos Taurus |
| isoform X1 | | | | | |
| Annexin A10 | XP_046772695.1 | Gallus gallus | Annexin A13 | NP 001098905.1 | Bos Taurus |
| isoform X2 | | | | | |
| Annexin A13 | XP_015138593.1 | Gallus gallus | Annexin A1 | NP_001157470.1 | Sus scrofa |
| isoform X1 | | | | | |
| Annexin A3 | XP_004009983.1 | Ovis aries | Annexin A2 | NP 001005726.1 | Sus scrofa |
| Annexin A6 | XP_004009036.1 | Ovis aries | Annexin A3 | XP 020957140.1 | Sus scrofa |
| isoform X1 | | | | | |
| Annexin A2 | A2SW69 | Ovis aries | Annexin A4 | NP 001161111.1 | Sus scrofa |
| Annexin A1 | XP_004004354.1 | Ovis aries | Annexin A5 | XP 003129266.2 | Sus scrofa |
| Annexin A4 | XP_004005861.1 | Ovis aries | Annexin A6 | XP 005672670.3 | Sus scrofa |
| Annexin A5 | XP_042107038.1 | Ovis aries | Annexin A7 | XP 005671157.1 | Sus scrofa |
| isoform X1 | | | isoform X1 | | |
| Annexin A5 | XP_012034784.2 | Ovis aries | Annexin A7 | XP 001927837.1 | Sus scrofa |
| isoform X2 | | | isoform X2 | | |
| Annexin A6 | XP_042106931.1 | Ovis aries | Annexin A8 | XP 020927616.1 | Sus scrofa |
| isoform X2 | | | isoform X1 | | |
| Annexin A6 | XP_042106932.1 | Ovis aries | Annexin A9 | XP 020944177.1 | Sus scrofa |
| isoform X3 | | | isoform X1 | | |
| Annexin A7 | XP_042096945.1 | Ovis aries | Annexin A9 | XP 013852614.1 | Sus scrofa |
| isoform X1 | | | isoform X2 | | |
| Annexin A7 | XP_042096947.1 | Ovis aries | Annexin A9 | XP 020944182.1 | Sus scrofa |
| isoform X2 | | | isoform X3 | | |
| Annexin A7 | XP_011960675.2 | Ovis aries | Annexin A9 | XP 020944183.1 | Sus scrofa |
| isoform X3 | | | isoform X4 | | |
| Annexin A8 | XP_004021595.3 | Ovis aries | Annexin A9 | XP 020944184.1 | Sus scrofa |
| | | | isoform X5 | | |
| Annexin A9 | XP_012036248.1 | Ovis aries | Annexin A9 | XP 020944185.1 | Sus scrofa |
| isoform X1 | | | isoform X6 | | |
| Annexin A10 | XP_042099506.1 | Ovis aries | Annexin A9 | XP 020944186.1 | Sus scrofa |
| | | | isoform X7 | | |
| Annexin A11 | XP_042096902.1 | Ovis aries | Annexin A10 | XP 003359116.1 | Sus scrofa |
| isoform x1 | | | | | |
| Annexin A11 | XP_027818135.1 | Ovis aries | Annexin A11 | XP 020929657.1 | Sus scrofa |
| isoform x2 | | | isoform X1 | | |
| Annexin A11 | XP_042096903.1 | Ovis aries | Annexin A11 | XP_005671204.1 | Sus scrofa |
| isoform x3 | | | isoform X2 | | |

isoform X1

| | | | | | |
|---|---|---|---|---|---|
| Annexin A13 | XP_042110093.1 | Ovis aries | Annexin A13 | XP 020944615.1 | Sus scrofa |
| isoform X1 | | | isoform X1 | | |
| Annexin A13 | XP_027828722.1 | Ovis aries | Annexin A13 | XP 020944616.1 | Sus scrofa |
| isoform X2 | | | isoform X2 | | |
| Annexin A4 | XP_010721503.1 | Meleagris | Annexin A11 | XP 043783035.1 | Cervus elaphus |
| isoform X1 | | gallopavo | | | |
| Annexin A4 | XP_010721504.1 | Meleagris | Annexin A13 | XP_043736162.1 | Cervus elaphus |
| isoform X2 | | gallopavo | isoform X1 | | |
| Annexin A5 | XP_010708237.1 | Meleagris | Annexin A13 | XP_043736163.1 | Cervus elaphus |
| | | gallopavo | isoform X2 | | |
| Annexin A6 | XP_003210442.2 | Meleagris | Annexin A13 | XP_043736164.1 | Cervus elaphus |
| isoform X1 | | gallopavo | isoform X3 | | |
| Annexin A6 | XP_010717451.1 | Meleagris | Annexin A1 | NP 001134743.1 | salmo salar |
| isoform X2 | | gallopavo | | | |
| Annexin A7 | XP_003208047.1 | Meleagris | Annexin A2 | XP_045544550.1 | salmo salar |
| isoform X1 | | gallopavo | | | |
| Annexin A7 | XP_010712699.1 | Meleagris | Annexin A2 | XP 014033065.1 | salmo salar |
| isoform X2 | | gallopavo | isoform X4 | | |
| Annexin A8 | XP_019473163.1 | Meleagris | Annexin A2 | XP 014033064.1 | salmo salar |
| isoform X1 | | gallopavo | isoform X3 | | |
| Annexin A8 | XP_019473165.1 | Meleagris | Annexin A2 | XP 014033062.1 | salmo salar |
| isoform X2 | | gallopavo | isoform X1 | | |
| Annexin A8 | XP_019473166.1 | Meleagris | Annexin A2 | XP_014033066.1 | salmo salar |
| isoform X3 | | gallopavo | isoform X5 | | |
| Annexin A10 | XP_003205430.1 | Meleagris | Annexin A2 | XP_014033063.1 | salmo salar |
| isoform X1 | | gallopavo | isoform X2 | | |
| Annexin A10 | XP_019470015.1 | Meleagris | Annexin A2 | XP 045565600.1 | salmo salar |
| isoform X2 | | gallopavo | isoform X6 | | |
| Annexin A11 | XP_010712435.1 | Meleagris | Annexin A3 | ACI67419.1 | salmo salar |
| | | gallopavo | | | |
| Annexin A13 | XP_010707588.1 | Meleagris | Annexin A4 | XP 013993876.1 | salmo salar |
| isoform X1 | | gallopavo | | | |
| Annexin A13 | XP_003205310.1 | Meleagris | Annexin A5 | ACN12538.1 | salmo salar |
| isoform X2 | | gallopavo | | | |
| Annexin A1 | XP_043747439.1 | Cervus elaphus | Annexin A6 | NP 001133223.1 | salmo salar |
| Annexin A2 | XP_043774895.1 | Cervus elaphus | Annexin A7 | XP 014011551.1 | salmo salar |
| Annexin A3 | XP_043762326.1 | Cervus elaphus | Annexin A11 | ACN11191.1 | salmo salar |
| Annexin A4 | XP_043773485.1 | Cervus elaphus | Annexin A11 | XP_014010477.2 | salmo salar |
| | | | isoform X1 | | |
| Annexin A5 | XP_043726893.1 | Cervus elaphus | Annexin A13 | XP_014036484.1 | salmo salar |
| isoform X1 | | | | | |
| Annexin A5 | XP_043726894.1 | Cervus elaphus | Annexin A2 | XP 044191957.1 | Thunnus |
| isoform X2 | | | | | albacares |
| Annexin A6 | XP_043769272.1 | Cervus elaphus | Annexin A4 | XP 044188828.1 | Thunnus |
| isoform X1 | | | | | albacares |
| Annexin A6 | XP_043769273.1 | Cervus elaphus | Annexin A6 | XP_044220639.1 | Thunnus |
| isoform X2 | | | isoform X1 | | albacares |
| Annexin A7 | XP_043781567.1 | Cervus elaphus | Annexin A6 | XP 044220640.1 | Thunnus |
| isoform X1 | | | isoform X2 | | albacares |
| Annexin A7 | XP_043781569.1 | Cervus elaphus | Annexin A13 | XP_044195526.1 | Thunnus |
| isoform X2 | | | isoform X1 | | albacares |
| Annexin A8 | XP_043782191.1 | Cervus elaphus | Annexin A13 | XP_044195528.1 | Thunnus |
| | | | isoform X2 | | albacares |
| Annexin A9 | XP_043734015.1 | Cervus elaphus | Annexin A1 | NP 001164623.1 | Oryctolagus |
| isoform X1 | | | | | cuniculus |
| Annexin A9 | XP_043734017.1 | Cervus elaphus | Annexin A2 | XP 051677885.1 | Oryctolagus |
| isoform X2 | | | | | cuniculus |
| Annexin A9 | XP_043734018.1 | Cervus elaphus | Annexin A3 | XP_008265912.1 | Oryctolagus |
| isoform X3 | | | | | cuniculus |
| Annexin A9 | XP_043734019.1 | Cervus elaphus | Annexin A4 | XP_051698784.1 | Oryctolagus |
| isoform X4 | | | isoform X1 | | cuniculus |
| Annexin A10 | XP_043747177.1 | Cervus elaphus | Annexin A4 | XP 017196184.1 | Oryctolagus |
| | | | isoform X2 | | cuniculus |
| Annexin A5 | XP_008266132.1 | Oryctolagus | Annexin A2 | XP 027321834.1 | Anas |
| | | cuniculus | isoform X2 | | platyrhynchos |
| Annexin A6 | XP_002710364.1 | Oryctolagus | Annexin A2 | XP 038040834.1 | Anas |
| isoform X1 | | cuniculus | isoform X3 | | platyrhynchos |
| Annexin A6 | XP_051699507.1 | Oryctolagus | Annexin A4 | XP 038022975.1 | Anas |
| isoform X2 | | cuniculus | | | platyrhynchos |
| Annexin A6 | XP_002710365.1 | Oryctolagus | Annexin A5 | XP 038034369.1 | Anas |
| isoform X3 | | cuniculus | isoform X1 | | platyrhynchos |
| Annexin A7 | XP_051679173.1 | Oryctolagus | Annexin A5 | XP 038034370.1 | Anas |
| isoform X1 | | cuniculus | isoform X2 | | platyrhynchos |
| Annexin A7 | XP_051679176.1 | Oryctolagus | Annexin A6 | XP 038042381.1 | Anas |
| isoform X2 | | cuniculus | | | platyrhynchos |
| Annexin A7 | XP_008268180.1 | Oryctolagus | Annexin A7 | XP 038037362.1 | Anas |
| isoform X4 | | cuniculus | isoform X1 | | platyrhynchos |
| Annexin A8 | NP_001075488.1 | Oryctolagus | Annexin A7 | XP 038037363.1 | Anas |
| | | cuniculus | isoform X2 | | platyrhynchos |
| Annexin A9 | XP_017201392.1 | Oryctolagus | Annexin A8 | XP_005022266.1 | Anas |
| | | cuniculus | | | platyrhynchos |
| Annexin A10 | XP_051698290.1 | Oryctolagus | Annexin A10 | XP_027312243.2 | Anas |
| | | cuniculus | | | platyrhynchos |
| Annexin A11 | NP_001076208.1 | Oryctolagus | Annexin A11 | XP 027315941.1 | Ana |
| | | cuniculus | | | platyrhynchos |
| Annexin A13 | XP_008253864.1 | Oryctolagus | Annexin A13 | XP_012956000.3 | Anas |
| isoform X1 | | cuniculus | isoform X1 | | platyrhynchos |
| Annexin A13 | NP_001075588.1 | Oryctolagus | Annexin A13 | XP 038030875.1 | Oryctolagus |
| | | cuniculus | isoform X2 | | cuniculus |
| Annexin A1 | XP_038026938.1 | Anas | Annexin A13 | XP_005020607.3 | Oryctolagus |
| | | platyrhynchos | isoform X3 | | cuniculus |
| Annexin A2 | XP_027321832.1 | Anas | | | |
| isoform X1 | | platyrhynchos | | | |

TABLE 2
Table 2. Additional Annexins.

| Accession No. | Annexin Name |
|---|---|
| AAI03376 | Annexin A1 |
| XP_004004354 | annexin A1 |
| AFN52410 | annexin A1 |
| P19619 | Annexin A1 |
| NP_001134743 | annexin A1 |
| DAA26880 | TPA: annexin A1 |
| Q92108 | Annexin A1 |
| P51662 | Annexin A1 |
| XP_019466051 | annexin A1 |
| AAX46348 | annexin I |
| NP_786978 | annexin A1 |
| XP_038026938 | annexin A1 |
| NP_996789 | annexin A1 |
| NP_001164623 | annexin A1 |
| NP_001157470 | annexin A1 |
| P46193 | Annexin A1 |
| XP_010724046 | annexin A1 |
| XP_043747439 | annexin A1 |
| ACI68668 | Annexin A1 |
| XP_051695263 | annexin A10 |
| EOB01970 | Annexin A10, partial |
| XP_003359116 | annexin A10 |
| XP_042099506 | annexin A10 |
| XP_046772695 | annexin A10 isoform X2 |
| XP_040555119 | annexin A10 isoform X1 |
| XP_003205430 | annexin A10 isoform X1 |
| XP_019470015 | annexin A10 isoform X2 |
| XP_027312243 | annexin A10 |
| DAA27104 | TPA: annexin A10-like |
| XP_046796403 | annexin A10 isoform X2 |
| XP_051698290 | annexin A10 |
| XP_040527760 | annexin A10 isoform X1 |
| XP_043747177 | annexin A10 |
| NP_001178987 | annexin A10 |
| XP_046775989 | annexin A11 isoform X1 |
| AID63998 | annexin A11, partial |
| ACN11191 | Annexin A11 |
| AID63986 | annexin A11, partial. |
| XP_042096903 | annexin A11 isoform X3 |
| XP_010712435 | annexin A11 |
| AID63991 | annexin A11, partial |
| NP_001133469 | Annexin A11 |
| JAA53744 | annexin A11 |
| NP_001076208 | annexin A11 |
| XP_046798761 | annexin A11 isoform X1 |
| XP_027315941 | annexin A11 |
| AID64010 | annexin A11, partial |
| AID63983 | annexin A11, partial |
| AAI19827 | ANXA11 protein |
| AID64004 | annexin A11, partial |
| ABI97371 | annexin A11, partial |
| XP_046798766 | annexin A11 isoform X1 |
| XP_046775996 | annexin A11 isoform X1 |
| NP_001012921 | annexin A11 |
| XP_046798757 | annexin A11 isoform X1 |
| XP_024842300 | annexin A11 isoform X3 |
| AID63984 | annexin A11, partial |
| XP_042096901 | annexin A11 isoform X1 |
| AID63996 | annexin A11, partial |
| XP_020929657 | annexin A11 isoform X1 |
| NP_776927 | annexin A11 |
| P27214 | Annexin A1l |
| AID64005 | annexin A11, partial |
| AID64009 | annexin A11, partial |
| XP_046775992 | annexin A11 isoform X1 |
| AID63981 | annexin A11, partial |
| AGV03641 | annexin A11, partial |
| XP_024842297 | annexin A11 isoform X1 |
| XP_024842299 | annexin A11 isoform X2 |
| AID64006 | annexin A11, partial |
| XP_001924213 | annexin A11 isoform X1 |
| AGV03625 | annexin A11, partial |
| AID64015 | annexin A11, partial |
| XP_042096899 | annexin A11 isoform X1 |
| AGV03640 | annexin A11, partial |
| XP_046798758 | annexin A11 isoform X1 |
| AID63990 | annexin A11, partial |
| AID64012 | annexin A11, partial |
| XP_003205310 | annexin A13 isoform X2 |
| NP_001186430 | annexin A13 |
| XP_014036483 | annexin A13 |
| XP_046766747 | annexin A13 isoform X1 |
| NP_001075588 | annexin A13 |
| XP_027828722 | annexin A13 isoform X2 |
| XP_010707588 | annexin A13 isoform X1 |
| DAA22796 | TPA: annexin A13 |
| XP_020944616 | annexin A13 isoform X2 |
| XP_015138593 | annexin A13 isoform X1 |
| XP_012956000 | annexin A13 isoform X1 |
| A0A1D5NUB1 | Annexin A13 |
| NP_001087257 | annexin A2 |
| XP_031411076 | annexin A2 |
| NP_001005726 | annexin A2 |
| AOP32373 | annexin A2 |
| XP_045565600 | annexin A2 isoform X6 |
| XP_040562137 | annexin A2 isoform X1 |
| AOP32369 | annexin A2 |
| AOP32380 | annexin A2 |
| XP_045544550 | annexin A2 |
| AAI02517 | Annexin A2 |
| AOP32379 | annexin A2 |
| AOP32371 | annexin A2 |
| XP_005659594 | annexin A2 isoform X1 |
| AOP32365 | annexin A2 |
| AOP32376 | annexin A2 |
| XP_042107854 | annexin A2 isoform X3 |
| AAU85387 | annexin A2 |
| XP_042107852 | annexin A2 isoform X1 |
| P04272 | Annexin A2 |
| AOP32370 | annexin A2 |
| XP_014033066 | annexin A2 isoform X5 |
| XP_014033065 | annexin A2 isoform X4 |
| XP_015134249 | annexin A2 isoform X1 |
| XP_043774895 | annexin A2 |
| XP_014033062 | annexin A2 isoform X1 |
| AOP32368 | annexin A2 |
| XP_013848366 | annexin A2 isoform X1 |
| NP_990682 | annexin A2 |
| XP_014033063 | annexin A2 isoform X2 |
| XP_025009571 | annexin A2 isoform X1 |
| XP_046780506 | annexin A2 isoform X1 |
| XP_024853052 | annexin A2 isoform X1 |
| P17785 | Annexin A2 |
| DAA25314 | TPA: annexin A2 |
| AOP32378 | annexin A2 |
| XP_051677885 | annexin A2 |
| XP_042107853 | annexin A2 isoform X2 |
| P19620 | Annexin A2 |
| AOP32367 | annexin A2 |
| NP_777141 | annexin A2 |
| AOP32372 | annexin A2 |
| XP_025009569 | annexin A2 isoform X1 |
| XP_014033064 | annexin A2 isoform X3 |
| AOP32377 | annexin A2 |
| AOP32364 | annexin A2 |
| AOP32374 | annexin A2 |
| Q2Q1M6 | Annexin A2 |
| XP_027321834 | annexin A2 isoform X2 |
| AOP32366 | annexin A2 |
| XP_046780505 | annexin A2 isoform X1 |
| AOP32381 | annexin A2 |
| XP_046780504 | annexin A2 isoform X1 |
| XP_038040834 | annexin A2 isoform X3 |
| XP_044191957 | annexin A2 |
| A2SW69 | Annexin A2 |
| ABB77206 | annexin A2 |
| AAX09027 | annexin A2 isoform 2 |
| XP_027321832 | annexin A2 isoform X1 |
| EOA95401 | Annexin A2, partial |
| XP_046780508 | annexin A2 isoform X1 |
| XP_014952166 | annexin A2 isoform X3 |
| XP_046780509 | annexin A2 isoform X1 |
| UEP53645 | annexin A2 |
| AOP32375 | annexin A2 |
| XP_046780507 | annexin A2 isoform X1 |
| XP_020957140 | annexin A3 |
| NP_001030402 | annexin A3 |
| Q3SWX7 | Annexin A3 |
| ACI67419 | Annexin A3 |
| NP_001134415 | annexin A3b |
| AAI04615 | Annexin A3 |
| XP_024848976 | annexin A3 isoform X2 |
| AFN52411 | annexin A3 |
| XP_043762326 | annexin A3 |
| XP_051676153 | annexin A3 |
| XP_024848975 | annexin A3 isoform X1 |
| XP_005208208 | annexin A3 isoform X1 |
| XP_042107475 | annexin A3 |
| XP_014952061 | annexin A3 |
| XP_008265912 | annexin A3 |
| DAA28512 | TPA: annexin A3 |
| XP_004009983 | Annexin A3 |
| P08132 | Annexin A4 |
| P13214 | Annexin A4 |
| XP_051698784 | annexin A4 isoform X1 |
| XP_013986531 | annexin A4 |
| XP_043773484 | annexin A4 |
| XP_004005861 | annexin A4 |
| NP_001161111 | annexin A4 |
| XP_042103999 | annexin A4 |
| XP_024998612 | annexin A4 |
| XP_046787730 | annexin A4 |
| AAI03382 | Annexin A4 |
| ACI69256 | Annexin A4 |
| XP_044188828 | annexin A4 |
| ABM06069 | annexin IV |
| XP_043773483 | annexin A4 |
| XP_038022975 | annexin A4 |
| XP_040545560 | annexin A4 |
| BAI47599 | annexin A4 |
| XP_010721504 | annexin A4 isoform X2 |
| XP_046759404 | annexin A4 |
| XP_004947704 | annexin A4 |
| XP_040545559 | annexin A4 |
| XP_046787729 | annexin A4 |
| XP_017196184 | annexin A4 isoform X2 |
| XP_042103998 | annexin A4 |
| ACI68517 | Annexin A4 |
| XP_038022974 | annexin A4 |
| XP_043773485 | annexin A4 |
| XP_042103995 | annexin A4 |
| XP_010721503 | annexin A4 isoform X1 |
| XP_013993876 | annexin A4 |
| EOB00059 | Annexin A4, partial |
| XP_046787731 | annexin A4 |
| DAA24519 | TPA: annexin A4 |
| ACI69495 | Annexin A4 |
| XP_046759403 | annexin A4 |
| XP_051698785 | annexin A4 isoform X2 |
| NP_001001440 | annexin A4 |
| XP_008266130 | annexin A5 |
| NP_001134508 | annexin A5 |
| P17153 | Annexin A5 |
| NP_001384241 | annexin A5 |
| AAI02236 | ANXA5 protein |
| XP_046772482 | annexin A5 isoform X1 |
| XP_038034370 | annexin A5 isoform X2 |
| EOB04292 | Annexin A5, partial |
| NP_001026709 | annexin A5 |
| NP_001035567 | annexin A5 |
| ACN12538 | Annexin A5 |
| NP_001384240 | annexin A5 |
| XP_012034784 | annexin A5 isoform X2 |
| XP_015326964 | annexin A5 isoform X1 |
| AAX09018 | annexin 5 |
| XP_043726893 | annexin A5 isoform X1 |
| XP_038034369 | annexin A5 isoform X1 |
| P81287 | Annexin A5 |
| NP_001384238 | annexin A5 |
| DAA28951 | TPA: annexin A5 |
| XP_045569789 | annexin A5 |
| XP_010708237 | annexin A5 |
| XP_046796267 | annexin A5 isoform X1 |
| XP_046796268 | annexin A5 isoform X1 |
| XP_043726894 | annexin A5 isoform X2 |
| NP_001384239 | annexin A5 |
| XP_046772481 | annexin A5 isoform X1 |
| ACN10184 | Annexin A5 |
| ABM06149 | annexin 5 |
| XP_008266131 | annexin A5 |
| XP_046796269 | annexin A5 isoform X1 |
| ACI67810 | Annexin A5 |
| XP_003129266 | annexin A5 |
| AFV58058 | annexin A5, partial |
| XP_014054784 | annexin A5 |
| XP_014067387 | annexin A5 |
| XP_008266132 | annexin A5 |
| XP_042107038 | annexin A5 isoform X1 |
| XP_046772480 | annexin A5 isoform X1 |
| F1NJI0 | Annexin A5 |
| XP_014068774 | annexin A6 isoform X2 |
| NP_001133223 | annexin A6 |
| XP_003210442 | annexin A6 isoform X1 |
| XP_002710365 | annexin A6 isoform X3 |
| XP_043769272 | annexin A6 isoform X1 |
| XP_010717451 | annexin A6 isoform X2 |
| XP_043769273 | annexin A6 isoform X2 |
| XP_014068773 | annexin A6 isoform X1 |
| AAT91808 | annexin A6 |
| XP_044220639 | annexin A6 isoform X1 |
| XP_043769271 | annexin A6 isoform X1 |
| XP_010805730 | annexin A6 isoform X1 |
| JAA74083 | annexin A6 tvl |
| XP_040502784 | annexin A6 isoform X2 |
| XP_005672670 | LOW QUALITY PROTEIN: annexin A6 |
| DAA27272 | TPA: annexin A6 |
| ACN10615 | Annexin A6 |
| XP_046782781 | annexin A6 isoform X2 |
| XP_010805732 | annexin A6 isoform X3 |
| XP_051699507 | annexin A6 isoform X2 |
| XP_042106932 | annexin A6 isoform X3 |
| XP_040502782 | annexin A6 isoform X1 |
| XP_024849644 | annexin A6 isoform X1 |
| ACH85263 | annexin A6 |
| NP_001384243 | annexin A6 isoform 2 |
| NP_990061 | annexin A6 isoform 1 |
| P79134 | Annexin A6 |
| P51901 | Annexin A6 |
| NP_001384242 | annexin A6 isoform 2 |
| AAI51392 | ANXA6 protein |
| XP_010805731 | annexin A6 isoform X2 |
| XP_004009036 | annexin A6 isoform X1 |
| NP_001096694 | annexin A6 |
| XP_002710364 | annexin A6 isoform X1 |
| XP_038042381 | LOW QUALITY PROTEIN: annexin A6 |
| Q9TS53 | Annexin A6 |
| XP_044220640 | annexin A6 isoform X2 |
| XP_005209618 | annexin A6 isoform X4 |
| XP_042106931 | annexin A6 isoform X2 |
| XP_046756240 | annexin A6 isoform X3 |
| XP_046782780 | annexin A6 isoform X1 |
| NP_001264276 | annexin A7 |
| XP_042096945 | annexin A7 isoform X1 |
| XP_043781567 | annexin A7 isoform X1 |
| XP_005671157 | annexin A7 isoform X1 |
| P20072 | Annexin A7 |
| XP_042096946 | annexin A7 isoform X1 |
| DAA14259 | TPA: annexin A7 |
| XP_040558337 | annexin A7 isoform X1 |
| XP_011960675 | annexin A7 isoform X3 |
| NP_001069459 | annexin A7 |
| XP_005226475 | annexin A7 isoform X1 |
| XP_043781569 | annexin A7 isoform X2 |
| XP_043781566 | annexin A7 isoform X1 |
| XP_014011551 | annexin A7 |
| XP_005226473 | annexin A7 isoform X1 |
| XP_038037363 | annexin A7 isoform X2 |
| AAI16142 | Annexin A7 |
| XP_043781570 | annexin A7 isoform X2 |
| XP_042096947 | annexin A7 isoform X2 |
| XP_001927837 | annexin A7 isoform X2 |
| XP_046776157 | annexin A7 isoform X1 |
| XP_003208047 | annexin A7 isoform X1 |
| XP_038037362 | annexin A7 isoform X1 |
| XP_005226474 | annexin A7 isoform X2 |
| XP_042096948 | annexin A7 isoform X3 |
| XP_010712699 | annexin A7 isoform X2 |
| XP_042096944 | annexin A7 isoform X1 |
| XP_051679173 | Annexin A7 isoform X1 |
| XP_051679176 | Annexin A7 isoform X2 |
| XP_008268180 | Annexin A7 isoform X4 |
| XP_040531194 | annexin A8 |
| XP_421646 | annexin A8 |
| XP_020927616 | annexin A8 isoform X1 |
| NP_776666 | annexin A8 |
| AAX46492 | annexin A8 |
| Q95L54 | Annexin A8 |
| O97529 | Annexin A8 |
| AAI13322 | Annexin A8 |
| EOA99380 | Annexin A8, partial |
| NP_001075488 | annexin A8 |
| XP_043782191 | annexin A8 |
| DAA14157 | TPA: annexin A8 |
| XP_015143930 | annexin A8 |
| XP_004021595 | annexin A8 |
| XP_005226585 | annexin A8 isoform X1 |
| AAX46493 | annexin A8 |
| NP_001230528 | annexin A8 |
| XP_040531195 | annexin A8 |
| XP_005022266 | annexin A8 |
| XP_019473163 | Annexin A8 isoform X1 |
| XP_019473165 | Annexin A8 isoform X2 |
| XP_019473166 | Annexin A8 isoform X3 |
| XP_043734017 | annexin A9 isoform X2 |
| XP_012036248 | annexin A9 isoform X1 |
| XP_043734015 | annexin A9 isoform X1 |
| NP_001030450 | annexin A9 |
| XP_010801336 | annexin A9 isoform X1 |
| XP_013852614 | annexin A9 isoform X2 |
| Q3ZC08 | Annexin A9 |
| AAX11401 | annexin A9 protein, partial |
| XP_020944178 | annexin A9 isoform X1 |
| XP_020944185 | annexin A9 isoform X6 |
| XP_042108924 | annexin A9 isoform X2 |
| XP_020944186 | annexin A9 isoform X7 |
| XP_020944177 | annexin A9 isoform X1 |
| XP_015318219 | annexin A9 isoform X1 |
| XP_020944184 | annexin A9 isoform X5 |
| AAX11400 | annexin A9 protein, partial |
| XP_015318237 | annexin A9 isoform X2 |
| NP_001230277 | annexin A9 |
| XP_017201392 | annexin A9 |
| XP_020944176 | annexin A9 isoform X1 |
| XP_043734019 | annexin A9 isoform X4 |
| XP_042108929 | annexin A9 isoform X2 |
| AAI02992 | Annexin A9 |
| XP_024845169 | annexin A9 isoform X1 |
| XP_020944181 | annexin A9 isoform X2 |
| XP_043734016 | annexin A9 isoform X1 |
| XP_010801338 | annexin A9 isoform X1 |
| XP_012036236 | annexin A9 isoform X1 |
| XP_010801340 | annexin A9 isoform X2 |
| XP_020944179 | annexin A9 isoform X1 |
| XP_020944182 | annexin A9 isoform X3 |
| DAA31676 | TPA: annexin A9 |
| XP_024845170 | annexin A9 isoform X1 |
| XP_043734018 | annexin A9 isoform X3 |
| XP_020944183 | annexin A9 isoform X4 |

Exemplary FABPs and retinoid binding proteins useful in the present disclosure are listed in Table 11 herein. Table 11 lists the name of the protein and the database designation number of the protein/gene. In embodiments, the FABP is a FABP listed in Table 11. In embodiments, the retinoid binding protein is a retinoid binding protein listed in Table 11. In embodiments, the FABP or retinoid binding protein is a FABP or retinoid binding protein from any animal source. In embodiments, the FABP or retinoid binding protein is a FABP or retinoid binding protein from any mammalian source. In embodiments, the FABP or retinoid binding protein is a FABP or retinoid binding protein from a non-human animal source. In embodiments, the FABP or retinoid binding protein is a FABP or retinoid binding protein from a bird source. In embodiments, the FABP or retinoid binding protein is a FABP or retinoid binding protein from a fish source. In embodiments, the FABP or retinoid binding protein is from chicken, duck, turkey, pig, sheep, cow, rabbit, deer, red deer, buffalo, water buffalo, salmon, cod, tilapia or tuna.

Depending on the host cell used, it may be helpful to select a heterologous protein that has a certain percentage of sequence identity to the protein derived from the host cell. In some embodiments, the heterologous protein has 0%, 1%, 2%, 3%, 4%, 5%, 6%. 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% sequence identity to a gene or a region of a gene derived from a host cell. In some embodiments the animal protein has 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% sequence identity to a protein or a fragment of a protein derived from a host cell.

In some embodiments, the amino acid sequence of the heterologous proteins may be modified by replacing one or more amino acids with a different amino acid (e.g., by changing the nucleotide sequence of the recombinant gene encoding the protein). Such amino acid modifications may improve the yield of the heterologous protein (e.g., by more robust protein expression) produced by the host cell that has been engineered to express the protein. Any amino acid modification can be made that improves or enhances the production of the animal proteins. In some embodiments a modification is made in the protein coding region of the heterologous protein gene. In other embodiments, a modification is made in a regulatory element that controls or modifies the heterologous protein gene. In embodiments, the heterologous protein gene is operably linked to a promoter, terminator or other regulatory sequence of the naturally-occurring heterologous protein gene. In embodiments, the nucleic acid coding region of the heterologous protein gene is operably linked to a promoter, terminator or other regulatory sequence other than those of the naturally-occurring heterologous protein gene. For example, the heterologous protein coding sequence may be under the regulatory control of a promoter, terminator or other regulatory sequence derived from the host cell.

Non-limiting examples of such amino acid modifications are: improving the efficiency of transcription and/or translation of the heterologous protein, improving the stability of the heterologous protein, altering the rate at which the protein is secreted by the host cell or modifying the activity of the heterologous protein to, for example, minimize any deleterious effects on the expression of the heterologous protein.

However, when a heterologous protein is intended to retain or substantially retain its natural function as a therapeutic or enzyme, modifications preferably should not detrimentally affect the desired activity of the heterologous protein. In heterologous proteins intended to be employed for nutrition, for example, in food compositions, where retention of natural function is not required, modifications can include any that minimize deleterious effects on the expression of the heterologous protein including those modifications that detrimentally affect the natural activity of the heterologous protein.

### Nucleic Acids Encoding Heterologous Proteins

Production of a recombinant heterologous protein of the disclosure can be achieved by the recombinant manipulation of a gene that encodes the heterologous protein, which is then inserted in a host cell expression system such that it expresses large amounts of a recombinant gene that is converted into the heterologous protein using the host cell expression system. This process can include the transcription of the recombinant DNA to messenger RNA (mRNA), the translation of mRNA into polypeptide chains, which optionally are ultimately folded into functional proteins and may be targeted to specific subcellular or extracellular locations depending on the sequence. However, a heterologous protein (nutritional protein) need not be folded or targeted to add to the nutritional value of a food product. Where the heterologous protein is a fragment or portion of a protein it may not be folded.

Genes encoding recombinant proteins can be obtained, for example, by taking a sample from a source of the protein and extracting nucleic acids, such as mRNA, from that sample and then amplifying the gene by reverse transcription followed by PCR. The sample could be a tissue sample (e.g., muscle), a blood sample, or a sample of mucus, skin, saliva, or hair. Another option is to have the gene synthesized by a company that performs such work.

Alternatively, where the genome sequence of the source organism (e.g., animal) has been determined, the gene sequences (DNA/nucleotide sequences) or protein sequences of a heterologous protein can be obtained by searching appropriate databases (e.g., UniProtKB and NCBI). A polynucleotide can be obtained using chemical synthesis, molecular cloning or recombinant methods, DNA or gene assembly methods, artificial gene synthesis, PCR, or any combination of those.

In the case that there are not sequences available for a heterologous protein of interest, known conserved regions of the protein or its gene can be used to amplify segments of the genes, and the flanking regions can be sequenced in order to obtain the full-length sequence. Multiple sequence alignments of a specific protein in several different organisms will show where the conserved regions lie, and which are the most suitable stretches of sequence to use for primer design. Primers with alternative nucleotides can be used when needed as is known in the art.

The present invention provides codon-optimized nucleic acid encoding a heterologous protein for expression in a host cell. Codon-optimization for expression in a particular host cell can be determined by codon usage tables or by using a program that is instructed by an algorithm that identifies a region of sequence that can be optimized for protein expression in the host cell. Any commercially available optimization algorithm or any publicly available algorithms can be used with the disclosure herein. Using such programs, various improvements can be achieved to enhance expression of a recombinant animal protein as discussed herein. Specific examples of codon-optimization of heterologous protein gene sequences for certain host cells are provided herein.

The gene sequences that can be used with the methods and compositions of the disclosure are those encoding the types of proteins described herein. In some embodiments, the gene sequence may include non-coding introns. In some embodiments, the gene sequences may not include non-coding introns.

Depending on what method is used to produce a recombinant animal protein, a gene encoding the heterologous protein may further comprise one or more regulatory elements. Nonlimiting examples of regulatory elements include but are not limited to a promoter, an enhancer, a signal sequence, a terminator, or a combination thereof. A gene encoding the heterologous protein may include one or more restrictions sites cleavable by a restriction enzyme. Additionally it is within the ordinary skill in the art to introduce one or more restriction sites into a nucleic acid sequence such as a gene. Restriction sites are useful in the construction and editing of heterologous nucleic acid sequences. For example, the presence of a restriction site can be used as an insertion point for an exogenous nucleic acid fragment. The presence of two spaced apart restriction sites can be used to excise nucleic acid sequence between the sites and optionally insert a different nucleic acid sequence in its place.

### Origin of the Heterologous Protein

The identification and cloning of proteins are discussed herein. The origin of the recombinantly expressed protein sequence (i.e., the species of organism (e.g., animal) from which the sequence to be recombinantly expressed is found in nature) can be any species within the biological kingdom. In embodiments, the origin of the protein is other than the host organism (i.e., is heterologous to the host) in which the protein is to be expressed.

In embodiments, the origin of the recombinantly expressed protein sequence is a vertebrate animal, which can be a fish, a bird, a mammal, an amphibian, or a reptile. The origin may be a placental mammal, monotreme mammal, or marsupial mammal (metatheria). The origin may furthermore be a bird or another vertebrate from the reptile clade. In some embodiments, the gene origin is a placental mammal, including but not limited to, carnivores (including lion, bear, weasel, seal, wolf, coyote, fox), equidae (including horse and donkey), even-toed ungulates (including pig, camel, cattle, and deer), Afrotheria (including elephants, woolly mammoth, golden moles, and manatees), and Boreoeutheria (including primates, rabbits, hares, pikas, rodents, moles, whales, bats, dogs, cats, seals, and hoofed mammals). In some embodiments, the origin is a monotreme mammal, including but not limited to platypus and echidna. In some embodiments, the origin is a marsupial mammal, including but not limited to, koala, possums, tapirs, kangaroos, wallabies, and marsupial lions. In some embodiments, the origin is a hoofed mammal, including but not limited to cattle, antelope, deer, reindeer, red deer, elk, sheep, goat, camels, carabao, yak, bison, buffalo, caribou, water buffalo, pig, horse, and donkey. In some embodiments, the origin is an endothermic vertebrate, classified as Aves, including but not limited to chicken, turkey, duck, pigeon, penguin, ostrich, goose, pheasant, and quail. In some embodiments, the gene origin is a reptile, including but not limited to, gila monsters and other lizards, alligators and crocodiles. In some embodiments, the gene origin is an aquatic animal, including but not limited to, shark, tuna, trout, salmon, herring, jacks, carp, catfish, cod, flounder, bass, tilapia, sturgeon, crab, lobster, shrimp, prawns, oysters, mussels, eels, shellfish, cuttlefish, starfish, crayfish, and jellyfish. In some embodiments, the gene origin is an amphibian, including but not limited to frogs, salamanders, and toads. In some embodiments, the gene origin is an insect.

In embodiments, the heterologous protein can be from a heterologous microorganism, e.g., a bacterium. In embodiments, the heterologous protein can be from a plant source, e.g., a crop source.

### Tissue Source

When the heterologous protein is from an animal the protein can be from any organ or tissue of an animal, including, but not limited to proteins expressed in the brain, skin, scales, feathers, eyes, shells, hair, horns, ears, liver, heart, kidney, stomach, intestines, and muscle tissue (e.g., skeletal, smooth or cardiac). In preferred embodiments, the recombinant animal protein is a muscle proteins. In some embodiments, the recombinant animal protein is cytoskeletal. In some embodiments, the actin cytoskeleton protein is a filament protein, a capping protein, an actin-binding protein, an actin-bundling protein, a monomer binding protein, a cytoskeletal linker protein, a membrane anchor protein, a stabilizing protein, a signaling protein, a capping protein, a severing protein, or a myosin. In some embodiments, the recombinant animal protein is a myosin. In some embodiments, the recombinant animal protein is an actin.

The animal muscle proteins include those proteins normally found in animal muscle tissue (or relatives of those proteins). In addition to myosin and actin, these proteins include, but are not limited to, troponin, tropomyosin, alpha-actinin, beta-actinin, titin, connectin, destrin, skeletal receptor, myosin-binding protein, desmin, leiomodin, tubulin, myotubularin, myozenin, telethonin, calsarcin, myotilin, nebulin, nebulin-related anchoring protein, myomesin, vinculin, paxillin, beta-enolase, myotubularin, calponin, caldesmon, transgelin, tropomodulin, supervillin, gelsolin, twinfilin, profilin, caveolin, catenin, cofilin, capping protein, leiomodin, tensin, M-protein, radixin, filamin, keratin, myopalladin, calsequestrin, caveolae-associated protein, nebulette, coronin, talin, dystrophin, dystroglycan, integrin, ankyrin, syncoilin, smoothelin-like-1, spectrin, synemin, paranemin, ponsin, plectin, skelemin, sarcoglycan, LINI protein, myoblast determination protein, myocyte-specific enhancer, and myocilin.

### Expression Vectors/Expression Cassettes

The disclosure also provides various expression vectors (e.g., constructs) comprising a genetic element (e.g., DNA, or cDNA) encoding for a heterologous protein (i.e., the protein of interest). Depending on the host cell used for protein expression, a person skilled in the art of molecular biology will know the appropriate expression vector to use (e.g., plasmid, virus) with the regulatory elements (e.g., transcriptional start site, promoter, and the like) and genetic elements required for protein expression in a particular host cell. Specific examples of expression vectors that can be used with the methods of the disclosure are provided herein. Herein, the term expression cassette includes a minimum of a coding sequence under the control of appropriate regulatory elements, typically at least a promoter and a terminator operably connected to the coding sequence such that the coding sequence is expressed under the control of the regulatory sequences. In embodiments, an expression cassette comprises a gene of interest (e.g., a heterologous gene) including any regulatory elements associated therewith. In embodiments, the expression cassette includes the coding sequence of a protein/polypeptide or fragment thereof with appropriate regulatory sequences derived from a different gene. In embodiments, an expression cassette includes the coding sequence of a protein/polypeptide or fragment thereof with appropriate regulatory sequences derived from the host organism into which the expression cassette will be introduced. In embodiments, expression vectors include integration vectors which are expression vectors which facilitate integration of at least one expression cassette into the genome of a host organism. In embodiments, an integration vector contains one or more genetic elements to facilitate integration of a portion of the vector containing at least one expression cassette into the genome of the host organism. In embodiments, the integration vector facilitates integration of at least one expression cassette into the host organism by homologous recombination. In embodiments, the integration vector facilitates integration of at least one expression cassette into a host yeast by homologous recombination. In an embodiment, an integration vector contains nucleic acid sequence including at least one expression cassette bounded by sequences which are homologous to a locus on the host genome to facilitate integration of the bounded nucleic acid sequence into the locus on the host genome. One of ordinary skill in the art understands the mechanism of integration into a host genome, particularly by homologous recombination, and knows how to construct integration vectors to achieve such integration particularly at a chosen locus in the genome. In embodiments, an integrative vector can include sequence to be integrated that on integration forms an expression cassette. The sequence integrated can include a coding sequence which on integration is placed under the regulatory control of a regulatory element present in the host genome at the integration site. Alternatively, the sequence integrated can include a regulatory element which on integration is positioned to exert regulatory control over a coding sequence present in the host genome at the integration site

In embodiments, this disclosure provides exemplary integration vectors for integration of heterologous genes into host genomes and particularly for integration into the yeast genome and more particularly in the genome of a strain of Saccharomyces. In embodiments, the integration vector contains additional genetic elements (e.g., host homologous sequences as noted above) to facilitate homologous recombination of a portion of the nucleic acid that is integrated into the host genome. Homologous recombination of such integrated sequences can be used to amplify the copy number of expression cassettes integrated into or formed on integration into the host genome. In embodiments, this disclosure provides exemplary integration vectors to amplify such expression cassettes in the host genome.

A genetic element is any coding or non-coding nucleic acid sequence. A genetic element can be a nucleic acid that codes for an amino acid, a peptide or a protein. Genetic elements may be operons, genes, gene fragments, promoters, exons, introns, regulatory sequences, or any combination of those. Genetic elements can include host homologous sequence which is useful for facilitating homologous recombination into a host genome. A genetic element can include an entire open reading frame of a protein, or the entire open reading frame and one or more (or all) regulatory sequences associated there with. The genes may be codon-optimized for expression in a particular recombinant host cell (e.g., codon-optimized for yeast, filamentous fungi, insect, or mammalian host cell). In embodiments, preferred host cells are yeast and filamentous fungi and more preferably are yeast and most preferably are strains of Saccharomyces, particularly Saccharomyces cerevisiae. In some embodiments, an expression vector can comprise one genetic element. In some embodiments, an expression vector can comprise at least 2, 3, 4, 5, or 6 genetic elements. In some embodiments, an expression vector can comprise one regulatory element. In some embodiments, an expression vector can comprise at least 2, 3, 4, 5, or 6 regulatory elements. A person skilled in the art knows the payload limitations (e.g. kilobase pairs) for certain various expression vectors (e.g., cosmids, plasmids, etc).

### Promoters

Disclosed herein are genetic constructs comprising a genetic element encoding a heterologous protein or part of a heterologous protein and the use thereof for the recombinant expression of the heterologous protein. Also disclosed are expression cassettes. The expression vector or expression cassette comprises at least one promoter and may comprise one or more additional promoters. A promoter may be a constitutive promoter, an inducible promoter, or a hybrid promoter. Where overexpression of a protein is toxic to a host cell (e.g., reduces growth of the cell, kills the cell, or reduces protein expression) it may be preferable to use an inducible promoter. Such inducible promoters are selectively inducible by selective application of a trigger to induce expression. In the expression vector, the gene construct and the method, the promoter may be a viral promoter, a prokaryotic promoter or a eukaryotic promoter. The promoter may be a synthetic promoter from a promoter library. The promoter may be any scientifically known promoter or a novel promoter. The promoter may be an engineered form of a known promoter or a hybrid promoter. The eukaryotic promoter may be a fungi promoter, a plant promoter, or an animal promoter. The fungal promoter may be the promoter of the genes phosphoglycerate kinase (PGK, PGK1, PGK3), enolase (ENO, ENO1, ENO2, ENO3), glyceraldehyde-3-phosphate dehydrogenase (gpdA, GAP, GAPDH), hexokinase, pyruvate decarboxylase, phosphofructokinase, glucose-6-phosphate isomerase, 3-phosphoglycerate mutase, pyruvate kinase, triosephosphate isomerase, phosphoglucose isomerase, glucokinase, alcohol dehydrogenase promoter (ADH1, ADH2, ADH4), isocytochrome C, acidic phosphatase, galactose metabolism enzymes, GAL (GAL1, GAL2, GAL3, GAL4, GAL5, GAL6, GAL7, GAL8, GAL9, GAL10), alternative oxidase (ADD), alcohol oxidase 1 (AOX1)), alcohol oxidase 2 (AOX2), CUP 1, AHSB4, adhl+, AINV, alcA, AXDH, cellobiohydrolase I (cbhl), ccg-1, cDNAl, cellular filament polypeptide (cfp), cpc-2, ctr4+, dihydroxyacetone synthase (DAS), FMD, formate dehydrogenase (FMDH), formaldehyde dehydrogenase (FLD1), GAA, GCW14, glucoamylase (glaA, gla-1), invl, isocitrate lyase (ICL1), glycerol kinase (GUT1), acetohydroxy acid isomeroreductase (ILV5), beta-galactosidase (lac4), LEU2, melO, MET3, MET25, KAR2, KEX2, methanol oxidase (MOX), nmtl, peroxin 8 (PEX8), pcbC, PET9, PHO5, PHO89, PYK1, phosphatidylinositol synthase (PIS1), RPS7, TEF, translation elongation factor I alpha (TEF1), sorbitol dehydrogenase (SDH), SSA4, THI1, homoserine kinase, XRP2, and YPTI, PHO5, CYC1, HIS3, ADC1, TAP1, URA3, LEU2, TRP1, TDH1, TDH3, FBA1, ADR1, TPI1, heat shock proteins (HSP26, HSP30, HSP31, HSP78, HSP82, and others), hexose transporters (HXT1, HXT2, HXT5, HXT7), maltose metabolism (MAL11, MAL12, MAL31, MAL32, MAL61, MAL62), or any combination of those.

A plant promoter may be the promoter of the gene phol, TPI, TPSI, and any combination of these. The animal promoter may be a heat-shock protein promoter, proactin promoter, immunoglobulin promoter, or the promoter of the gene B2, HSP82, Ser 1, triose phosphate isomerase (TPI), or any combination of those. However, any promoters can be used if they drive the expression of recombinant proteins in a particular host cell.

In embodiments, the promoter is a promoter of a haploinsufficient gene of a fungus, particularly a haploinsufficient gene of a yeast. In specific embodiments, the promoter is a promoter of a haploinsufficient gene of a strain of Saccharomyces and particularly of Saccharomyces cerevisiae.

### Selective Gene Marker

The expression vector, including the integration vector, may include a selective gene marker. For example, an expression vector may comprise an auxotrophic marker. Non-limiting examples of auxotrophic markers that can be used with the disclosure include trp1, leu2, his3, ade1, arg4, his4, ura3, and/or met2. In some embodiments, more than one selection gene marker may be used. In some embodiments, the expression vector may comprise a selectable marker, which may be an antibiotic resistance gene. The resistance gene may confer resistance to drugs including, but not limited to, zeocin, ampicillin, blasticidin, kanamycin, nourseothricin, chloramphenicol, tetracycline, triclosan, ganciclovir, or derivatives thereof. In some embodiments, more than one resistance genes may be used. Yet, for applications to food compositions, it may be desirable not to use an antibiotic resistance gene in the method for producing recombinant heterologous protein.

In applications when there are several heterologous proteins expressed, it may be useful to use one or more resistance genes in combination with one or more auxotrophic markers.

### Integration and Transformation

The compositions of the invention include a recombinant host cell transformed with an expression vector to express one or more recombinant animal proteins. One or more expression cassettes or expression vectors with the required genetic elements (e.g., regulatory elements or protein-encoding, genetic elements) may be integrated into a genome. In some applications, it may be desirable to integrate multiple copies of the same expression cassette or expression vector. Alternatively, or in addition, the host cell may comprise multiple copies of an expression vector, where the expression vector is not integrated into a genome.

Any small DNA molecule within a cell that is capable of being physically separated from chromosomal DNA and can replicate can be used with the methods and compositions of the disclosure. The expression vectors that can be used with the disclosure are a plasmid, a conjugative plasmid, a non-conjugative plasmid, a cosmid, a hybrid plasmid, a virus, a phage, or the like. Host cells may be transformed or transduced to introduce the expression vector by transfection, infection, endocytosis, F-mating, mating, PEG-mediated protoplast fusion, Agrobacterium tumefaciens-mediated transformation, chemical transformation, electroporation, heat-shock transformation, biolistic transformation or any other method known in the art.

### Signal Peptide Sequence

The expression vector may further comprise a signal peptide sequence. A signal peptide, also known as a, signal sequence, targeting signal, localization signal, localization sequence, secretion signal, transit peptide, leader sequence, or leader peptide, may cause extracellular secretion of a protein or initiate transport of the protein. Extracellular secretion of a recombinant animal protein from a host cell simplifies protein purification. Recovery of a recombinant protein from a cell culture supernatant may be preferable to lysing host cells to release a complex mixture of proteins including intracellular proteins of the host cell. For some applications, secretion may reduce harmful effects that intracellular overexpression of a recombinant animal protein may have on a host cell such as toxicity or reduced growth rate. Secretion may produce higher amounts of a recombinant protein compared to intracellular expression. Secretion of a protein may also enable posttranslational modification (e.g., glycosylation) or aid in folding the protein correctly and allow for the formation of disulfide bonds. In embodiments, secretion of heterologous protein is not required or preferred.

### Host Cells

The expression vectors provided by the disclosure are transformed into host cells. Typically, the host cell is a eukaryotic host cell. Any eukaryotic host cell known in the art can be used with the expression vectors and animal proteins provided by the disclosure to make a recombinant host cell. Examples of a eukaryotic host cell that can be used with the disclosure are an insect cell, a fungal cell, a plant cell, and a mammalian cell. In embodiments herein, the host cell is a filamentous fungal cell or a yeast cell. Genetic modification of the host cell is accomplished in one or more steps via the design and construction of appropriate vectors and transformation of the host cell with those vectors. Electroporation and/or chemical (such as calcium chloride- or lithium acetate-based) transformation methods can be used. Methods for transforming yeast strains are described in WO99/14335, WO00/71738, WO02/42471, WO03/102201, WO03/102152 and WO03/049525; these methods are generally applicable for transforming host cells in accordance with this invention. The DNA used in the transformations can either be cut with particular restriction enzymes or used as circular DNA. The recombinant host cells can be cultured in appropriate media to produce large quantities of the heterologous protein

### Fungal Host Cells

In some embodiments, the host cell used to express the protein is a fungal host cell. The fungal cell can be filamentous fungus or yeast. In some applications, the fungal host cell is a wild-type yeast. However, often, the fungal host cell used with the method and compositions of the disclosure is a modified fungal host cell (e.g., through mutation, genome shuffling, protoplast fusion, cytoduction, etc.) to enhance the production or yield of heterologous protein, aid selection of, or any other modification that enhances production of heterologous protein such that the host cell gives more robust expression. The modification can result in a fungal host cell that is polyploid or aneuploid. In some applications, the host cell may be modified so that it grows faster, grows to a higher cell density, is less sensitive to environmental factors in the bioproduction process fluctuations, such as an unexpected change in temperature or reduced nutrients. The fungal host cell may be obtained from a variety of sources known to a person of ordinary skill in the art, including commercial sources. In some embodiments, the fungal host cell may be selected from the “Saccharomyces Yeast Clade”, as described in US Publication No. 2009/0226991.

In embodiments, the host cell is a yeast. In certain embodiments, the yeast host cell is a Saccharomyces sensu stricto yeast. The term “Saccharomyces sensu stricto” taxonomy group is a cluster of yeast species that are highly related to Saccharomyces cerevisiae (Rainieri et al., 2003, J. Biosci Bioengin 96: 1-9). Saccharomyces cerevisiae extracts (from spent yeast) have been commercialized as food supplement for years. Among their several claims, the application as protein source is highlighted. In fact, their high protein content (about 45-60%) including essential amino acids with high biological value, safety and low cost. Oliveira, et al., Valorisation of protein-rich extracts from spent brewer's yeast (Saccharomyces cerevisiae): an overview. Biomass Conv. Bioref. (2022). Saccharomyces sensu stricto yeast species include, but are not limited to, S. cerevisiae, S. kudriavzevii, S. mikatae, S. bayanus, S. uvarum, S. carocanis and hybrids derived from these species (Masneuf et al., 1998, Yeast 7: 61-72). An ancient whole genome duplication (WGD) event occurred during the evolution of the hemiascomycete yeast and was discovered using comparative genomic tools (Kellis et al., 2004, Nature 428: 617-24; Dujon et al., 2004, Nature 430:35-44; Langkjaer et al., 2003, Nature 428: 848-52; Wolfe et al., 1997, Nature 387: 708-13). Using this major evolutionary event, yeast can be divided into species that diverged from a common ancestor following the WGD event (termed “post-WGD yeast” herein) and species that diverged from the yeast lineage prior to the WGD event (termed “pre-WGD yeast” herein). In some embodiments, the yeast host cell may be selected from a post-WGD yeast genus, including, but not limited to Saccharomyces and Candida. In some embodiments, post-WGD yeast species include: S. cerevisiae, S. uvarum, S. bayanus, S. paradoxus, S. castelli, and C. glabrata. In some embodiments, the yeast host cell may be selected from a pre-whole genome duplication (pre-WGD) yeast genus including, but not limited to Saccharomyces, Kluyveromyces, Candida, Pichia, Issatchenkia, Debaryomyces, Hansenula, Yarrowia and, Schizosaccharomyces. Representative pre-WGD yeast species include: S. kluyveri, K thermotolerans, K. marxianus, K. waltii, K. lactis, C. tropicalis, P. pastoris, P. anomala, P. stipitis, I. orientalis, I. occidentalis, I. scutulata, D. hansenii, H. anomala, Y. lipolytica, and S. pombe.

A yeast host cell used with the disclosure may be either Crabtree-negative or Crabtree-positive, as described in US Publication No. 2009/0226991. A yeast microorganism may be either Crabtree-negative or Crabtree-positive. A yeast cell having a Crabtree-negative phenotype is any yeast cell that does not exhibit the Crabtree effect. The term “Crabtree-negative” refers to both naturally occurring and genetically modified organisms. Briefly, the Crabtree effect is defined as the inhibition of oxygen consumption by a microorganism when cultured under aerobic conditions due to the presence of a high concentration of glucose (e.g., 50 g glucose L⁻¹). In other words, a yeast cell having a Crabtree-positive phenotype continues to ferment irrespective of oxygen availability due to the presence of glucose, while a yeast cell having a Crabtree-negative phenotype does not exhibit glucose mediated inhibition of oxygen consumption. In some embodiments, the yeast host cell may be selected from yeast with a Crabtree-negative phenotype including, but not limited to, the following genera: Saccharomyces, Lachancea, Kluyveromyces, Pichia, Jssatchenkia, Komagataella, Yarrowia, Hansenula, Debaromyces, Ogataea, Zygosaccharomyces and Candida. Crabtree-negative species include, but are not limited to: L. kluyveri (formerly known as S. kluyveri), K. lactis, K. marxianus, P. anomala, S. stipitis (formerly known as P. stipitis), I. orientalis, D. occidentalis, P. scutulata, P. anomala, Ogataea polymorpha, Arxula adeninivorans, Cyberlindnera jadinii, K. phaffii, Y. lipolytica, Kluyveromyces fragilis, D. hansenii, P. kudriavzevii and C. utilis. In some other embodiments, the yeast host cell may be selected from yeast with a Crabtree-positive phenotype, including, but not limited to, the genera Saccharomyces, Kluyveromyces, Zygosaccharomyces, Naumovozyma, Lachancea, Dekkera, Candida, Pichia and Schizosaccharomyces. Crabtree-positive yeast species include, but are not limited, to: S. cerevisiae, S. uvarum, S. bayanus, S. paradoxus, N. castellii, L. thermotolerans, C. glabrata, Z. bailii, Z. rouxii, D. bruxellensis and S. pombe.

Another characteristic may include the property that the host cell is non-fermenting. In other words, it cannot metabolize a carbon source anaerobically while the yeast is able to metabolize a carbon source in the presence of oxygen. Non-fermenting yeast refers to both naturally occurring cells and recombinant cells. In some embodiments, the recombinant host cells may be host cells that are non-fermenting yeast host cells, including, but not limited to those classified into a genus selected from the group consisting of Tricosporon, Rhodotorula, Myxozyma, or Candida. In a specific embodiment, the non-fermenting yeast is C. xestobii.

Cultured mammalian cell lines may also be used to express the heterologous proteins provided by the disclosure. In some embodiments, Chinese hamster ovary (CHO) can be used. In some embodiments, human cell lines such as HEK or HeLa may be used to produce heterologous protein. In some embodiments, a commercially available mammalian expression system can be used such as Expi293, ExpiCHO, T-REx Expression System, Flp-In T-REx system, or GeneSwitch System from Thermofisher.

### Methods for Bioproduction, Cell Culture Processes and Fermentation

The bioproduction of a recombinant protein may be conducted by cell culture processes or by fermentation. When fermentation is used, it may be conducted aerobically, microaerobically or anaerobically. In some embodiments, the method for producing a recombinant protein for any purpose, but particularly for food product production, comprises (i) providing a reactor or flask comprising a fungal colony and (ii) a feedstock comprising a nitrogen-containing material and a carbon-containing material (e.g., sugar), and permitting the fungal colony to grow in presence of the feedstock to yield the fungus-containing product comprising a recombinant protein. In some embodiments, a selective media or reagent can be used to select for host cells harboring the recombinant gene. In some embodiments, the method for producing a recombinant protein for any purpose, and particularly for food product production comprises (i) providing a reactor comprising a fungal colony and (ii) a feedstock comprising a nitrogen-containing material and a sugar-containing material, and (iii) when the fungal colony reaches the exponential growth phase, an inducing agent is added to yield the fungus-containing product comprising a recombinant animal protein. In some embodiments, the fungal colony comprises one or more budding fungi. Examples of preferred budding fungi are Saccharomyces cerevisiae, Schizosaccharomyces pombe, Komagataella phaffii, Kluyveromyces lactis, and a derivative thereof. In some embodiments, the genome of a budding fungi can be genetically modified in at least one gene to yield more robust protein expression. Genetic modifications that can yield more robust protein expression are discussed herein. In some embodiments, the genome of a budding fungi can be genetically modified to be protease deficient.

In some embodiments, the fungal colony comprises one or more filamentous fungi. Non-limiting examples of filamentous fungi that can be used are Aspergillus oryzae, Trichoderma reesei, Fusarium venenatum, Geotrichum candidum, Penicillium camemberti, Penicillium roqueforti, and a derivative thereof. In some embodiments, the genome of a filamentous fungi can be genetically modified in at least one gene to yield more robust protein expression. Genetic modifications that can yield more robust protein expression are discussed herein. In some embodiments, the genome of a filamentous fungi can be genetically modified to be protease deficient. In some embodiments, the recombinant animal protein is produced in a recombinant host cell and expressing the recombinant protein intracellularly. In some embodiments, the recombinant protein is produced in a recombinant host cell and expressing the recombinant protein such that it is secreted into the culture broth.

The recombinant animal protein may be obtained by a whole-cell preparation (i.e., host cell itself, and the recombinant protein expressed within or on its surface, can be added to the food composition), a protein concentrate preparation, or by isolating an animal protein. Depending on where the protein is expressed in the cell (e.g., extracellularly or intracellularly) protein concentrate can be from a cell lysate or a cell supernatant after centrifugation. In embodiments, recombinant protein is purified after production by any known method. It will be appreciated that recombinant therapeutic protein will be purified employing methods known in the art appropriate for pharmaceutically active materials.

### Additional Aspects of Methods for Fermentation and Downstream Process

There are different methods for producing recombinant proteins using genetically engineered fungi, particularly through optimized fermentation modes designed to maximize protein yield and overall productivity while maintaining process scalability and improve process control. These fermentation modes include batch, fed-batch, continuous, and draw-and-fill (semi-continuous) fermentation and the recombinant protein expression can be driven by different promoters that may or may not require a specific mode of induction, which is described below and known in the art.

Recombinant protein expression can be induced by the addition of specific inducers, by the depletion of specific compounds, or by process changes like temperature, pH, or anaerobic shifts. Protein production can also take place without specific inducers. Different promoters may be used to target different stages of the growth process. The fungus cells grow and produce recombinant protein until nutrients are depleted or the culture reaches a desired phase, at which point the process ends, and the product is harvested. For systems where induction is temperature- or pH-dependent, the temperature may be shifted up or down, in the range of 40° C. to 20° C., such as 20° C., 22° C., 24° C., 26° C., 28° C., 30° C., 32° C., 34° C., 36° C., 38, or 40° C., or the pH adjusted from 6.5 to 4.0, once the desired biomass is reached. Anaerobic fermentation may be used to induce expression under low-oxygen conditions. This embodiment is designed to maximize recombinant protein expression while minimizing metabolic burden, ensuring high intracellular protein content without significantly compromising cell viability.

In one embodiment, the method comprises a batch fermentation process. A complete culture medium is charged into the bioreactor, providing all necessary nutrients for fungus (including yeast) growth and recombinant protein production at the beginning of the process. Typical components of the medium include a carbon source, such as glucose, corn syrup, or glycerol, in concentrations ranging from 20 to 300 g/L, such as 20 to 200 g/L, 20 to 150 g/L, and 20 to 100 g/L; nitrogen sources like ammonium sulfate or yeast extract, in concentrations of 1 to 20 g/L; salts like magnesium sulfate (0.1 to 10 g/L), potassium phosphate (1 to 20 g/L), calcium chloride (0.05 to 2 g/L), sodium chloride (0.05 to 3 g/L); and trace elements, such as zinc, iron, and manganese, boron, iodine, molybdenum, or copper, in concentrations ranging from 0.01 to 1,000 mg/L, such as 0.1 to 1,000 mg/L, 1 to 1,000 mg/L, 10 to 1,000 mg/l, 100 to 1,000 mg/L, 0.1 to 100 mg/L, 0.1 to 500 mg/L, 1 to 500 mg/L, 10 to 500 mg/L, 20 to 100 mg/L, 25 to 100 mg/L, or 25 to 75 mg/L, 1 to 10 mg/L, 1 to 20 mg/L, 5 to 10 mg/L, 0.1 to 2 g/L, 0.1 to 1 g/L, 0.1 to 10 g/L, 0.05 to 2 g/L, 0.05 to 3 g/L, 0.05 to 1 g/L, or 0.05 to 0.5 g/L, such as 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, or 500 mg/L. The medium may also include vitamins, such as biotin, pantothenic acid, folic acid, inositol, nicotinic acid, p-aminobenzoic acid, pyridoxine, riboflavin, or thiamine, in concentrations of 0.1 to 1000 mg/L, such as 0.1 to 1,000 mg/L, 1 to 1,000 mg/L, 10 to 1,000 mg/l, 100 to 1,000 mg/L, 0.1 to 100 mg/L, 0.1 to 500 mg/L, 1 to 500 mg/L, 10 to 500 mg/L, 20 to 100 mg/L, 25 to 100 mg/L, or 25 to 75 mg/L, 1 to 10 mg/L, 1 to 20 mg/L, 5 to 10 mg/L, 0.1 to 2 g/L, 0.1 to 1 g/L, 0.1 to 10 g/L, 0.05 to 2 g/L, 0.05 to 3 g/L, 0.05 to 1 g/L, or 0.05 to 0.5 g/L, such as 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, or 500 mg/L.

In another embodiment, the method utilizes fed-batch fermentation, which addresses the limitations of batch fermentation by allowing nutrients to be fed incrementally throughout the process. Initially, a base medium is introduced, with a lower concentration of nutrients to avoid substrate inhibition. Typical components of the medium include a carbon source, such as glucose, corn syrup, or glycerol, in concentrations ranging from 10 to 50 g/L, such as 10 to 40 g/L, 10 to 30 g/L or 10 to 20 g/L; nitrogen sources like ammonium sulfate or yeast extract, in concentrations of 1 to 20 g/L, such as 1 to 15 g/L, 1 to 10 g/L, 1 to 5 g/L, 2.5 to 20 g/L, 2.5 to 15 g/L, 2.5 to 10 g/L, or 2.5 to 5 g/L, such as 1.75, 2.75, 3.75, 4.75, or 5.75 g/L; salts like magnesium sulfate (0.1 to 10 g/L), such as 0.5 to 10 g/L, 1 to 10 g/L, such as 1, 2, 3, 4, 5, 6, or 7 g/L, potassium phosphate (1 to 20 g/L), such as 1 to 15 g/L, 5 to 20 g/L, 5 to 15 g/L, such as 10.5, 11.5, 12.5, 13.5, 14.5, or 15.5 g/L, calcium chloride (0.05 to 2 g/L), such as 0.1 to 2 g/L, 0.1 to 1 g/L, 0.05 to 1 g/L, 0.05 to 0.5 g/L, such as 0.1, 0.2, 0.3, 0.4, 0.5, 0.6, 0.7, 0.8, or 0.9 g/L, sodium chloride (0.05 to 3 g/L), such as 0.05 to 2 g/L, 0.1 to 3 g/L, 0.1 to 2 g/L, 0.05 to 1 g/L, 0.05 to 0.5 g/L, such as 0.1, 0.2, 0.3, 0.4, 0.5, 0.6, 0.7, 0.8, or 0.9 g/L; and trace elements, such as zinc, iron, and manganese, boron, iodine, molybdenum, or copper in concentrations ranging from 0.01 to 1,000 mg/L, such as 0.1 to 1,000 mg/L, 1 to 1,000 mg/L, 10 to 1,000 mg/l, 100 to 1,000 mg/L, 0.1 to 100 mg/L, 0.1 to 500 mg/L, 1 to 500 mg/L, 10 to 500 mg/L, 20 to 100 mg/L, 25 to 100 mg/L, or 25 to 75 mg/L, 1 to 10 mg/L, 5 to 10 mg/L, 0.1 to 2 g/L, 0.1 to 1 g/L, 0.05 to 1 g/L, or 0.05 to 0.5 g/L, such as 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, or 500 mg/L. The medium may also include vitamins, such as biotin, pantothenic acid, folic acid, inositol, nicotinic acid, p-aminobenzoic acid, pyridoxine, riboflavin, or thiamine, in concentrations of 0.1 to 1000 mg/L, such as 0.1 to 1,000 mg/L, 1 to 1,000 mg/L, 10 to 1,000 mg/l, 100 to 1,000 mg/L, 0.1 to 100 mg/L, 0.1 to 500 mg/L, 1 to 500 mg/L, 10 to 500 mg/L, 20 to 100 mg/L, 25 to 100 mg/L, or 25 to 75 mg/L, 1 to 10 mg/L, 5 to 10 mg/L, 0.1 to 2 g/L, 0.1 to 1 g/L, 0.05 to 1 g/L, or 0.05 to 0.5 g/L, such as 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, or 500 mg/L. As the process proceeds, additional nutrients, particularly carbon sources such as glucose, corn syrup, or glycerol in concentrations ranging from 400 to 700 g/L, such as 400 to 600 g/L, 500 to 700 g/L, 450 to 550 g/L, such as 400, 450, 500, 550, 600, 650, or 700 g/L, based on final fed amounts, are fed into the bioreactor in controlled amounts to maintain growth and avoid nutrient depletion. Salts like magnesium sulfate, potassium phosphate (1 to 20 g/L), calcium chloride (0.05 to 2 g/L), sodium chloride (0.05 to 3 g/L); and trace elements, such as zinc, iron, and manganese, boron, iodine, molybdenum, or copper, in concentrations ranging from 0.01 to 1,000 mg/L such as 0.1 to 1,000 mg/L, 1 to 1,000 mg/L, 10 to 1,000 mg/l, 100 to 1,000 mg/L, 0.1 to 100 mg/L, 0.1 to 500 mg/L, 1 to 500 mg/L, 10 to 500 mg/L, 20 to 100 mg/L, 25 to 100 mg/L, or 25 to 75 mg/L, 1 to 10 mg/L, 1 to 20 mg/L, 5 to 10 mg/L, 0.1 to 2 g/L, 0.1 to 1 g/L, 0.1 to 10 g/L, 0.05 to 2 g/L, 0.05 to 3 g/L, 0.05 to 1 g/L, or 0.05 to 0.5 g/L, such as 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, or 500 mg/L, may be fed with the carbon source or as an alternate feed. The feed may also include vitamins, such as biotin, pantothenic acid, folic acid, inositol, nicotinic acid, p-aminobenzoic acid, pyridoxine, riboflavin, or thiamine, in concentrations of 0.1 to 1000 mg/L, such as 0.1 to 1,000 mg/L, 1 to 1,000 mg/L, 10 to 1,000 mg/i, 100 to 1,000 mg/L, 0.1 to 100 mg/L, 0.1 to 500 mg/L, 1 to 500 mg/L, 10 to 500 mg/L, 20 to 100 mg/L, 25 to 100 mg/L, or 25 to 75 mg/L, 1 to 10 mg/L, 1 to 20 mg/L, 5 to 10 mg/L, 0.1 to 2 g/L, 0.1 to 1 g/L, 0.1 to 10 g/L, 0.05 to 2 g/L, 0.05 to 3 g/L, 0.05 to 1 g/L, or 0.05 to 0.5 g/L, such as 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, or 500 mg/L.

Carbon sources may be fed at rates of 0.05 to 30 g/h/L0, where L0 is the initial fermentation volume. The carbon feed rate may be increased as needed based on biomass growth, such as a linear increase until the maximum feed rate is achieved, such as increasing from 0.05 to 30 g/h/L0, from 1 to 30 g/h/L0, from 0.1 to 20 g/h/L0, from 0.1 to 30 g/h/L0, from 0.5 to 20 g/h/L0, from 0.6 to 15 g/h/L0, from 0.7 to 14 g/h/L0, from 0.8 to 13 g/h/L0, from 0.9 to 12 g/h/L0, or from 1 to 11 g/h/L0. Nitrogen, salts, trace elements and vitamins may be added based on biomass growth. Induction typically occurs once the biomass reaches a certain level, typically when the optical density at 600 nm (OD600) is between 20 and 500.

Fed-batch fermentation is typically monitored using optical density (OD) measurements, glucose concentration sensors, dissolved oxygen (DO) levels, or off-gas analysis, ensuring that nutrient addition is optimized for maximum protein production. Off-gas analysis involves the continuous measurement of gases evolved from the fermentation vessel, specifically carbon dioxide (CO₂) and oxygen (O₂), to infer various aspects of metabolism and growth. This technique enhances process control and efficiency by providing real-time data on metabolic activity.

The present invention also provides a continuous fermentation method for the production of recombinant proteins, wherein fresh culture medium is continuously introduced into the bioreactor while an equal volume of spent medium, along with the recombinant protein, is removed. This continuous flow of medium ensures that the cells remain in a steady growth phase, which facilitates prolonged recombinant protein production over an extended period.

The culture medium typically contains a carbon source, such as glucose, corn syrup, or glycerol, in concentrations ranging from 10 to 50 g/L. In some embodiments, the carbon source concentration ranges from 10 to 40 g/L, from 10 to 30 g/L, or from 10 to 20 g/L, depending on the specific needs of the fermentation process. Additionally, during the fermentation process, higher concentrations of carbon sources are added, typically ranging from 400 to 700 g/L. In some embodiments, these concentrations range from 400 to 600 g/L, from 500 to 700 g/L, or from 450 to 550 g/L. Individual values for the carbon concentration may include 400, 450, 500, 550, 600, 650, or 700 g/L.

Nitrogen sources such as ammonium sulfate or yeast extract are provided in the medium in concentrations ranging from 1 to 20 g/L. In some embodiments, the nitrogen source concentration ranges from 1 to 15 g/L, from 1 to 10 g/L, or from 1 to 5 g/L. Alternatively, nitrogen sources may be used in smaller ranges, such as from 2.5 to 20 g/L, from 2.5 to 15 g/L, from 2.5 to 10 g/L, or from 2.5 to 5 g/L. Specific concentrations may also be utilized, including 1.75, 2.75, 3.75, 4.75, or 5.75 g/L, depending on the fermentation requirements.

The culture medium may also contain salts necessary for maintaining cell stability and metabolic activity. For example, magnesium sulfate may be included at concentrations ranging from 0.1 to 10 g/L, with more specific ranges of 0.5 to 10 g/L or 1 to 10 g/L, such as 1, 2, 3, 4, 5, 6, or 7 g/L. Potassium phosphate may be included in the medium in concentrations ranging from 1 to 20 g/L, with additional ranges of 1 to 15 g/L, from 5 to 20 g/L, or from 5 to 15 g/L. Specific concentrations may include 10.5, 11.5, 12.5, 13.5, 14.5, or 15.5 g/L. Calcium chloride may be added at concentrations ranging from 0.05 to 2 g/L, with alternative ranges including 0.1 to 2 g/L, 0.1 to 1 g/L, or 0.05 to 0.5 g/L. Individual concentrations may include 0.1, 0.2, 0.3, 0.4, or 0.5 g/L. Similarly, sodium chloride may be used at concentrations ranging from 0.05 to 3 g/L, with alternative ranges such as 0.05 to 2 g/L, 0.1 to 3 g/L, or 0.05 to 0.5 g/L. Specific concentrations of sodium chloride may include 0.1, 0.2, 0.3, 0.4, 0.5, 0.6, 0.7, 0.8, or 0.9 g/L.

Trace elements, essential for various enzymatic functions and cellular growth, are also included in the medium. These trace elements are typically provided at concentrations ranging from 0.01 to 1,000 mg/L. In some embodiments, the concentrations range from 0.1 to 1,000 mg/L, from 1 to 1,000 mg/L, from 10 to 1,000 mg/L, or from 100 to 1,000 mg/L. Other embodiments may include trace element concentrations ranging from 0.1 to 100 mg/L, from 0.1 to 500 mg/L, from 1 to 500 mg/L, or from 10 to 500 mg/L. Specific ranges may include from 25 to 100 mg/L, from 1 to 10 mg/L, or from 5 to 10 mg/L. Typical trace elements in the medium include zinc, iron, manganese, boron, iodine, molybdenum, and copper, with concentrations as low as 0.1 mg/L and as high as 1 g/L, depending on the specific fermentation requirements.

The culture medium may further include vitamins necessary for cell metabolism, such as biotin, pantothenic acid, folic acid, inositol, nicotinic acid, p-aminobenzoic acid, pyridoxine, riboflavin, or thiamine. These vitamins are typically added at concentrations ranging from 0.1 to 1,000 mg/L. More specific concentrations may range from 1 to 1,000 mg/L, from 10 to 500 mg/L, or from 25 to 100 mg/L. In some embodiments, the vitamin concentration may range from 0.05 to 1 g/L, or from 0.05 to 0.5 g/L. Individual values may include 0.1, 0.2, 0.3, 0.4, or 0.5 g/L.

Throughout the continuous fermentation process, it is critical to maintain tight control over key parameters such as nutrient levels, pH, biomass, and dissolved oxygen (DO). The feed rate of the medium is controlled to maintain a constant dilution rate. The specific dilution rate can vary depending on the particular fungus strain and recombinant protein being produced. In some embodiments, the dilution rate is maintained between 0.05 to 0.5/h. In further embodiments, the dilution rate may range from 0.1 to 0.4/h, from 0.15 to 0.35/h, or from 0.2 to 0.3/h. In specific embodiments, the dilution rate may be set at values such as 0.05, 0.1, 0.15, 0.2, 0.25, 0.3, 0.35, 0.4, 0.45, or 0.5/h, depending on the specific microorganism used and the desired recombinant protein production rate. The optimal dilution rate is selected to maintain the cells in a steady-state growth phase, which enhances recombinant protein yield and overall process stability.

Additionally, the invention describes a draw-and-fill (semi-continuous) fermentation method. In this mode, the process operates like batch fermentation for a set period, during which cells grow and produce recombinant protein. After a portion of the culture is harvested, fresh medium is added to replenish the nutrients and continue fermentation. The initial medium typically contains carbon sources (10 to 50 g/L), nitrogen sources (1 to 20 g/L), salts, trace elements, and vitamins, similar to batch and fed-batch processes. The replenishment medium is added at similar concentrations, but tailored based on the consumption of the prior batch. By removing a portion of the culture and refilling with fresh medium, the process can extend production time without the complexity of a fully continuous system.

Another important factor is the management of nutrient supply and waste removal. In high-density systems, the fungi rapid growth consumes nutrients quickly and produces metabolic byproducts that can inhibit further growth if not properly managed. Ensuring a continuous or semi-continuous supply of essential nutrients, such as carbon sources, nitrogen, salts, trace elements, and vitamins, is crucial. This might involve implementing fed-batch or continuous feeding strategies to maintain optimal nutrient levels. Additionally, regular monitoring and removal of waste products, such as ethanol or organic acids, is necessary to prevent their accumulation and adverse effects on fermentation performance. Effective process monitoring, including the use of sensors for glucose levels, pH, and temperature, coupled with automated control systems, helps maintain the desired fermentation conditions and supports high-density fungus fermentations. Across all fermentation modes, careful monitoring of key parameters is essential for optimizing protein yield and ensuring product quality.

One of the primary considerations is the control of oxygen supply. In high-density aerobic fermentations, fungus cells consume oxygen rapidly, and inadequate oxygen can lead to incomplete aerobic respiration, overflow metabolism, and reduced overall efficiency. This necessitates the precise regulation of dissolved oxygen (DO) levels, which can be achieved through the use of spargers, oxygen enrichment, and careful monitoring with online sensors. Maintaining DO levels typically between 10% and 40%, such as between 10% and 30%, between 20% and 40%, between 10% and 20%, or between 20% and 30%, saturation is essential to support robust cell growth and high productivity while avoiding the risk of oxidative stress or anaerobic fermentation.

pH control is maintained at a range of 4.5 to 7.0, such as 4.5 to 6.0, 4.5 to 5.5, 4.5 to 5.0, 5.0 to 7.0, 5.0 to 6.5, or 5.0 to 6.0, such as 4.5, 5.0, 5.5, 6.0, 6.5, or 7.0, depending on the specific fungi strain and product requirements, often adjusted using acid or base addition, such as the addition of perchloric acid, hydroiodic acid, hydrobromic acid, hydrochloric acid, sulfuric acid, nitric acid, oxalic acid, sulfurous acid, phosphoric acid, nitrous acid, hydrofluoric acid, methanoic acid, benzoic acid, ethanoic acid, carbonic acid, hydrosulfuric acid, hypochlorous acid, hydrocyanic acid, boric acid, sodium hydroxide, potassium hydroxide, magnesium hydroxide, calcium hydroxide, or ammonium hydroxide. Biomass concentration is typically monitored using optical density (OD) at 600 nm. Glucose or glycerol concentrations are monitored continuously or periodically, with online sensors, high-performance liquid chromatography (HPLC), or a bioanalyzer, used to ensure that feeding rates are optimized for the production process.

To increase the protein yield, high cell density cultures are crucial. This is achieved through careful control of growth phases and nutrient supply. Carbon sources such as glucose, fructose, sucrose, mannitol, ethanol, lactose, maltose, xylose, sorbitol, and glycerol are fed at optimized rates, such as 0.05 to 30 g/h/L₀, 1 to 30 g/h/L₀, 0.1 to 20 g/h/L₀, 0.1 to 30 g/h/L₀, 0.5 to 20 g/h/L₀, 0.6 to 15 g/h/L₀, 0.7 to 14 g/h/L₀, 0.8 to 13 g/h/L₀, 0.9 to 12 g/h/L₀, or 1 to 11 g/h/L₀ to promote fungus growth while avoiding substrate inhibition and overflow metabolism (e.g., ethanol production). Nitrogen sources, such as ammonium, are typically maintained in concentrations ranging from 0.1 to 10 g/L, such as 0.1 to 5 g/L, 0.5 to 5 g/L, 1 to 5 g/L, 0.1 to 2 g/L, 0.5 to 2 g/L, such as 0.5, 1, 1.5, 2, 2.5, 3, or 3.5 g/L. Oxygen supply is critical, and dissolved oxygen (DO) is typically kept between 10% and 40% saturation through controlled air or oxygen sparging.

The invention provides embodiments that utilize various alternative carbon sources for high-density fungus fermentations, tailored to optimize fungus growth, recombinant protein production, and overall process efficiency.

In one embodiment, sucrose is employed as an alternative carbon source in high-density fermentation processes. Sucrose is hydrolyzed into glucose and fructose by fungus cells, which are subsequently utilized for growth and protein production. This embodiment is particularly useful when utilizing fungus strains with high sucrose assimilation capabilities. The addition of sucrose could decrease substrate inhibition and supports high cell densities. The concentrations fed or batched are comparative to glucose and glycerol.

In one embodiment, molasses is used as a complex carbon source in high-density fermentations. The complex mixture in molasses provides various sugars including glucose, fructose, and sucrose, as well as essential nutrients and minerals. Fungus strains efficiently utilize these sugars for growth and protein production. The inclusion of molasses helps achieve high biomass concentrations, while also reducing the cost of carbon sources. The fermentation process is monitored for optimal nutrient levels, and any potential inhibitory effects are managed by adjusting molasses concentration. The concentrations fed or batched are comparative to glucose and glycerol.

In one embodiment, corn syrup is utilized as a complex carbon source. The syrup, which contains a mixture of glucose, maltose, and oligosaccharides, provides a readily available carbon source that supports rapid fungus growth and high-density fermentation. The use of corn syrup facilitates efficient protein production and allows for precise control over the carbon supply. This embodiment is advantageous for processes requiring consistent and high-quality carbon sources, with corn syrup being adjusted based on the specific fermentation requirements. The concentrations fed or batched are comparative to glucose and glycerol.

Intracellular accumulation of recombinant proteins can lead to cellular stress, which must be managed to maintain cell viability and enhance protein yield. Controlling environmental conditions such as temperature (typically 25° C. to 33° C., such as 27° C. to 33° C., 29° C. to 33° C., 25° C. to 31° C., 25° C. to 30° C., such as 25° C., 26° C., 27° C., 28° C., 29° C., 30° C., 31° C., 32° C., or 33° C.) and pH (between 4.5 and 7, such as 4.5 to 6.0, 4.5 to 5.5, 4.5 to 5.0, 5.0 to 7.0, 5.0 to 6.5, or 5.0 to 6.0, such as 4.5, 5.0, 5.5, 6.0, 6.5, or 7.0) helps reduce protein misfolding and aggregation. Limiting the accumulation of byproducts, such as ethanol, is also important for maintaining healthy fungus cultures, which can be achieved by controlling carbon feed rates (e.g., maintaining glucose concentrations below 0.5 g/L, such as below 0.4 g/L, below 0.3 g/L, below 0.2 g/L, below 0.1 g/L, or below 0.05 g/L).

The recombinant protein or proteins may be produced intracellularly. In those cases, lysis of the fungus cells may be required in order to recover the protein or proteins. Mechanical disruption methods such as high-pressure homogenization or bead milling are typically employed, with conditions optimized to minimize protein damage. Enzymatic treatments using lytic enzymes such as zymolyase or lyticase may be used to further improve cell wall breakdown. Post-lysis, the protein is recovered through standard purification methods such as filtration, centrifugation, and chromatography, depending on the specific characteristics of the protein.

Carbon sources, such as glucose, are controlled to prevent overflow metabolism. In glucose-based systems, the concentration of glucose is maintained below 0.5 g/L, such as below 0.4 g/L, below 0.3 g/L, below 0.2 g/L, below 0.1 g/L, or below 0.05 g/L, during the production phase to avoid ethanol formation, which could cause stress. This embodiment is designed to maintain optimal intracellular conditions for protein folding, thereby enhancing overall protein quality and reducing cellular damage during production.

In aspects, nutrient composition and metabolic pathways are optimized to favor intracellular protein production. The carbon-to-nitrogen ratio in the medium is adjusted to favor protein synthesis over biomass accumulation during the later stages of fermentation. The carbon source is fed at 10 to 40 g/L/h, while nitrogen is supplemented at a rate of 0.05 to 1 g/L/h based on biomass growth.

In aspects of the invention, to further enhance protein synthesis, amino acids such as arginine, lysine, or methionine are supplemented in the medium at concentrations ranging from 0.01 to 1 g/L, such as 0.01 to 0.02, 0.01 to 0.05, 0.02, 0.06, 0.04 to 0.1, 0.08 to 1 g/L, depending on the metabolic needs of the recombinant protein.

In aspects of the invention, specific metabolic pathways can be knocked out or downregulated to divert resources toward recombinant protein production. For example, in Saccharomyces cerevisiae, deletion of the pyruvate decarboxylase gene (PDC1) can prevent ethanol formation, improving flux toward the TCA cycle and protein synthesis pathways.

In aspects of the invention, the feed rate of nutrients, such as carbon sources or growth media, can be varied dynamically based on the growth phase of the fungus culture in order to maximize productivity. This adaptive feeding strategy ensures that the fungus cells are supplied with the appropriate nutrients at each stage of their growth, thereby enhancing both biomass accumulation and product formation.

In aspects, during the initial lag phase, cells are acclimating to the fermentation environment, and metabolic activity is low. In this phase, minimal nutrient feeding is required. The feed rate is kept low, often limited to just maintenance levels, to prevent nutrient excess, which could cause stress or waste resources. Typical feed rates during this phase range between 0.01 to 0.1 g/L/h for carbon sources like glucose or glycerol.

In aspects, once the culture enters the exponential growth, the cells exhibit rapid biomass accumulation and increased metabolic activity. To support this accelerated growth, the feed rate is increased significantly. In this phase, the carbon source and other nutrients are fed at rates that match the fungi consumption rate, typically ranging from 1 to 40 g/L/h. The feed rate is carefully controlled to avoid substrate inhibition, while ensuring that nutrient availability does not become limiting. Continuous monitoring of dissolved oxygen (DO), pH, and substrate concentration may be used to fine-tune the feed rate.

As the culture approaches the stationary phase, growth slows down, and the focus may shift from biomass accumulation to product formation, such as recombinant protein production. During this transition, the feed rate is gradually reduced to prevent the buildup of byproducts like ethanol, which can inhibit both growth and product formation. Typical feed rates during this transition phase are between 0.2 to 1 g/L/h, such as 0.2 to 0.3, 0.2 to 0.4, 0.4 to 0.8, 0.4 to 1, 0.6 to 1, 0.8 to 1 g/L/h, depending on the specific fungus strain and fermentation objectives.

In the stationary phase, fungus growth reaches a plateau, and nutrient consumption decreases. To maximize productivity during this phase, the feed rate is reduced further, often to maintenance levels, to avoid nutrient waste and prevent excess accumulation of metabolic byproducts. However, if product formation (e.g., protein secretion) is still active, the feed rate may be adjusted to maintain optimal metabolic conditions for product yield. Feed rates in this phase typically range from 0.05 to 0.2 g/L/h, such as 0.05 to 0.08, 0.1, 0.05 to 00.9, 0.06 to 0.1, 0.05 to 0.15, 0.09 to 0.18, 0.08 to 0.2 g/L/h, ensuring that the fungus cells have just enough resources to continue producing the target compound without excessive growth or byproduct formation.

To optimize the feed rate dynamically across all growth phases, real-time monitoring of key process parameters, such as biomass concentration, oxygen demand, CO₂ production, and substrate levels, is employed. A feedback control system adjusts the feed rate in response to changes in these parameters. For instance, a sudden drop in dissolved oxygen may indicate excessive growth or substrate accumulation, prompting a reduction in feed rate, while a drop in CO₂ evolution rate may signal the need to increase the feed rate.

This variable feed strategy ensures that the fungus culture remains in an optimal state throughout the fermentation process, thereby maximizing productivity in terms of both biomass and product formation.

In aspects, a heat kill step is employed to deactivate the fungi cells after fermentation is complete, ensuring microbial stability and preventing contamination in the final product. The heat kill step typically involves heating the fermentation medium to a temperature between 80° C. and 100° C., such as between 80° C. and 95° C., between 80° C. and 90° C., between 85° C. and 95° C., or between 85° C. and 90° C., such as 80° C., 85° C., 90° C., 95° C., or 100° C. for a duration of 10 to 180 minutes, such as 20 to 180 minutes, 30 to 180 minutes, 60 to 180 minutes, 90 to 180 minutes, 120 to 180 minutes, 30 to 120 minutes, 30 to 90 minutes, 30 to 60 minutes, 60 to 180 minutes, 60 to 120 minutes, or 60 to 90 minutes, such as 10, 20, 30, 60, 90, 120, 150, or 180 minutes. The exact parameters may vary depending on the specific fungus strain, medium composition, and desired final product characteristics. This thermal treatment not only inactivates the fungus but also minimizes the risk of spoilage organisms, ensuring product safety and extending shelf life. Additionally, the controlled application of heat prevents undesirable alterations in the sensory and functional qualities of the product. Another alternative, which may be preferred, is to heat at a high temperature and short time (HTST). HTST treatment usually operates at higher temperatures, often between 121-150° C. for a short period, typically 10 to 60 seconds. This higher temperature range is critical for processes where fungus concentrations are high, ensuring complete microbial inactivation while maintaining the desired quality in the final product.

In aspects of the invention, a downstream process for dewatering and drying fungus cells at large scale is performed to ensure efficient separation and removal of water from the biomass while maintaining cell viability and integrity. This process is applicable to high-density fungus fermentations where large quantities of fungus biomass are produced, and subsequent dewatering and drying steps are critical to obtaining a final product suitable for storage, transportation, or further processing.

In aspects, when the fermentation process is complete, the fungus culture is subjected to a dewatering step, where the bulk of the liquid medium is removed. This can be achieved through large-scale centrifugation, freezing, electro-osmosis, or evaporation. The fungus slurry is fed into a continuous centrifuge system, such as a disk-stack or decanter centrifuge, capable of handling high throughputs. The centrifugal force separates the fungus cells from the surrounding fermentation broth by sedimenting the cells into a concentrated paste while the clarified supernatant is removed. Typical centrifuge speeds range from 3,000 to 10,000 RPM, depending on the equipment and the fungus strain used. The fungus concentration in the resulting paste can be increased to 15-30% dry matter, such as 15-25%, 15-20%, or 20-25%, such as 15%, 16%, 17%, 18%, 19%, 20%, 25%, or 30%, reducing the water content and preparing the biomass for the drying step.

In aspects of the invention, large-scale filtration techniques such as rotary drum vacuum filters or membrane filtration may be employed for dewatering. The fungus slurry is passed through a rotating filter drum or membrane filter, which captures the fungus cells on the filter surface while allowing the liquid to pass through. The fungus cake formed on the filter surface can reach similar dry matter content (15-30%) as with centrifugation. Filtration systems are often used in tandem with centrifugation to further concentrate the fungus biomass if required, or in cases where centrifugation alone is insufficient for certain strains or fermentation conditions.

In another embodiment, tangential flow filtration (TFF) is used. TFF offers a scalable process with lower shear forces, ensuring high yield and fungus viability. The fungus culture is circulated through a TFF unit to remove liquid and concentrate the microbial biomass. The TFF module uses membranes with pore sizes suitable for separating fungus cells from the broth. A pump controls the flow, and the feed reservoir handles cultures with appropriate densities. The transmembrane pressure and cross-flow velocity are controlled to optimize filtration without damaging the cells, allowing efficient water removal and concentration of the fungus culture.

Once dewatered, the concentrated microbial paste undergoes a large-scale drying process to reduce the moisture content and enhance shelf life. Spray drying is employed in this embodiment, where the fungus paste is atomized into fine droplets using a spray nozzle or rotary atomizer. These droplets are then introduced into a stream of hot air inside the drying chamber, where the moisture evaporates rapidly, leaving behind dried fungus particles. The inlet air temperature typically ranges from 150° C. to 200° C., such as from 150° C. to 175° C., 175° C. to 200° C., 160° C. to 190° C., or 170° C. to 180° C., such as 150° C., 155° C., 160° C., 165° C., 170° C., 175° C., 180° C., 185° C., 190° C., 195° C., or 200° C. while the outlet air temperature is controlled between 70° C. and 90° C., such as 70° C., 75° C., 80° C., 85° C., or 90° C. to prevent thermal damage to the fungus cells. Spray drying is highly efficient for large-scale operations, allowing for continuous processing and producing a dried fungus product with a moisture content of less than 3-8%, such as less than 4-7% or less than 5-6%, such as less than 3%, 4%, 5%, 6%, 7%, or 8%.

In an alternative embodiment, drum drying is used for large-scale drying of fungus biomass. The fungus paste is applied as a thin film onto the surface of a rotating drum, which is heated from within by steam. As the drum rotates, the water in the fungus film evaporates due to the heat, and the dried fungus film is scraped off using a stationary blade. The temperature of the drum surface is maintained between 120° C. and 170° C. to ensure efficient drying without damaging the cells. Drum drying is particularly suited for producing flakes or powders and is highly efficient in terms of energy usage and throughput for large-scale applications.

In yet another embodiment, fluidized bed drying is employed as an alternative to spray or drum drying. The dewatered cell paste is placed in a fluidized bed dryer, where hot air is passed through the bed, causing the cell particles to become suspended in the air stream. The fluidization of the particles ensures uniform drying and efficient heat transfer. Fluidized bed drying is particularly suitable for large-scale processes because it can handle high volumes of material and allows for precise control over temperature and moisture content, ensuring a consistent final product with minimal thermal degradation.

This embodiment of the invention thus provides an integrated and scalable downstream process for the dewatering and drying of fungus biomass, optimized for large-scale production while maintaining product quality and efficiency.

### Food Products

In embodiments, heterologous protein is employed in the production of food product or a food composition.

### Harvesting of Intermediate Food Product

The disclosure also provides methods for making an intermediate food product (also referred to herein as an intermediary food product). In some embodiments, the method comprises culturing eukaryotic host cells, particularly filamentous fungi and yeast cells, that recombinantly express a heterologous protein, particularly animal proteins, and harvesting the recombinant host cell, thereby making an intermediate food product. In some embodiments, the method comprises culturing a eukaryotic host cell that recombinantly expresses a heterologous protein (e.g., nutritive protein), concentrating the recombinant host cell culture, extracting proteins in a protein concentrate from the concentrated culture, thereby making an intermediate food product. In some embodiments, the method comprises culturing a eukaryotic host cell that recombinantly expresses a heterologous protein, concentrating the recombinant host cell from the culture, and isolating the heterologous protein, thereby making an intermediate food product.

### Cell is a Filamentous Fungi or a Yeast.

Where the heterologous protein is expressed intracellularly in the host cell, a cell lysate can be obtained from the eukaryotic host cell to make the intermediate food product. Where the heterologous protein is expressed extracellularly, the cell supernatant can be obtained the intermediate food product. The intermediate food product can also be made in a format such that it is used to another food product. In some embodiments, the intermediate food product is harvested and made in the format of an ingredient, a coating, a palatability agent, or a flavoring agent as discussed in more detail below.

### Intermediate Food Products/Food Ingredient

The disclosure also provides various intermediary food products/food ingredients comprising the recombinant protein. The intermediary food product can be substantially free of an antibiotic, a growth hormone, animal meat, or proteins derived from animal meat. The recombinant protein can be harvested and provided to the intermediary food product as a whole-cell food composition, a protein concentrates food composition, or as a protein isolate food composition. An intermediate food product can be mixed, coated, soaked or injected into an ultimate ingestible food product. The ultimate ingestible food product can be a commercially available feed, food, supplement, or treat. In embodiments recombinant animal proteins are preferred for use in food products.

In some embodiments, the intermediary food is a wet, semi-moist, or dry ingredient that is added to another food product. The intermediary food can also be a coating to be added to the exterior of a food product. The coating can be soaked, brushed, or sprayed on a food product. In some embodiments, the intermediary food protein can be a palatability agent that enhances the acceptance of the food product, as a flavoring agent or agent that enhances mouth-feel (e.g., texture and the like). In some embodiments, the harvested whole cell, protein concentrate, or protein isolate can be concentrated and dried, thereby making a dry intermediate food product. A dry intermediate food product comprising the recombinant animal protein can be in the form of a powder, a granule, a pellet, a slurry or paste, of varying moisture content.

### Food Product Composition

The disclosure provides various food product compositions (for humans and pets and other non-human animals) comprising the recombinant animal protein as well as supplements. The food product can be substantially free of an antibiotic, an animal growth hormone, animal meat, or proteins derived from animal meat. In some embodiments, the food product is substantially free of any other ingredient. In other embodiments, the food product is combined with other ingredients. The recombinant protein-containing food product can be formulated as a primary diet food product for an animal or individual (e.g., that is, it acts as the core source of daily nutrition). Examples of a primary food product include, but are not limited to, a meal, a kibble, a semi-moist food, a wet food, a dry food (e.g., freeze-dried or dehydrated). The recombinant protein-containing food product can be formulated as secondary diet food product (that is, it does not provide nutrients in the amounts that are required for daily nutrition for an animal or individual). Examples of a secondary diet food product are a snack, a treat, or an edible toy. The recombinant protein-containing food product can also be made from an intermediary diet food product (e.g., ingredient, a coating, a palatability agent, or a flavoring agent) that is added to make an ultimate ingestible food product.

The recombinant protein is introduced into a dry, semi-moist, or wet food composition by addition of the intermediate food product, which can be a whole-cell food product, a protein concentrate food product, or as a protein isolate food product, thereby making a dry food product. In some embodiments, the dry food product can be further processed and shaped into a kibble, a treat, a snack, a chew, or an edible toy. In some other embodiments, intermediate food product, which can be a whole cell, protein concentrate, or protein isolate can be concentrated, dried, and then rehydrated with one or more wet ingredients thereby making a wet food product. Wet products comprising the recombinant animal protein can be in the form of a slurry, a paste, a suspension, or a liquid. The wet food composition maybe semi-moist, intermediate moist, or moist. In some embodiments, the wet food composition can be further processed and shaped into a kibble, a treat, a snack, a chew, or a toy.

Depending on the percentage of essential amino acids desired for a food composition one can determine the amount of intermediate food product needed to achieve the desired amino acid content in the final food product (e.g. dry or wet food product). For example, the contribution of amino acids from a protein of known amino acid composition can be calculated for different expression levels.

### Whole-Cell Food Products

The disclosure also provides a whole-cell food product composition. The whole-cell food product composition is made with the host cell expressing the recombinant protein. Host cells expressing recombinant protein may be harvested by batch centrifugation, continuous flow centrifugation, filter press, flocculation, rotary drum vacuum filtration, tangential flow filtration, ultrafiltration or combination of these methods or any technique known in the art. Cells may be lysed by raising temperature, autolysis, by high-pressure homogenization (e.g., French press), ultrasonic cavitation, bead beating, rotor-stator processors, freeze-thaw cycles, enzymatic lysis (e.g., lysozyme, lysostaphin, zymolase, cellulose, protease or glycanase), osmotic shock methods, chemical lysis (by alkaline, detergent or organic solvent) or a combination of these methods or any technique known in the art. In some embodiments, food product comprising the recombinant animal protein is a whole-cell food product. In some embodiments, the whole-cell food composition comprises about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, or 90% of recombinant protein by dry weight, semi-moist weight, or wet weight.

### Protein Concentrate Food Products

The disclosure also provides protein concentrate food product compositions. In some embodiments, the protein concentrate food product comprising the recombinant protein is made from a protein concentrate from a host cell expressing the recombinant protein. Depending on whether the protein is expressed intracellularly or extracellularly in the host cell, the protein can be harvested from a cell lysate or cell supernatant of the host cell, respectively. A protein concentrate can be purified from a host cell lysate or host cell supernatant by any technique known in the art. [In some embodiments, the protein isolate food composition comprises about 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, or 90% of recombinant animal protein by dry weight, semi-moist weight, or wet weight.

### Protein Isolate Food Products

The disclosure also provides protein isolate food product compositions. In some embodiments, the protein isolate food product comprising the recombinant protein is made from a protein isolate from a host cell expressing the recombinant protein. Where a protein isolate is desired, the gene encoding the protein will often further comprise a molecule tag or label that can facilitate the isolation of the animal protein. In some embodiments, one or more tags or labels can be used to isolate different animal proteins expressed in the same host cell. Depending on if the protein is expressed intracellularly or extracellularly in the host cell, the protein can be harvested from a cell lysate or cell supernatant of the host cell, respectively. The proteins can be isolated using techniques known in the art. In some embodiments, the protein isolate food composition comprises about 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, or 90% of recombinant protein by dry weight.

### Other Ingredients

A recombinant protein of the disclosure may be combined with other ingredients such as fats, carbohydrates, supplemental non-recombinant proteins, fiber, nutritional supplements (e.g., minerals, and vitamins) to make a food composition. In some embodiments, the recombinant protein of the disclosure may be combined with other ingredients to make a food product that meets the nutritional requirements of an animal (i.e., a nutritionally balanced food product). In some embodiments, the recombinant protein of the disclosure may be combined with other ingredients to make a food product more palatable to an animal or an individual. In some embodiments, the recombinant protein of the disclosure may be combined with other ingredients to meet the nutritional requirements of an animal and to make it more palatable to an animal or an individual.

### Amino Acids

For some food compositions, such as a primary diet food, it may be desirable to combine the recombinant protein with additional amino acids. Any amino acid that makes a food composition nutritionally balanced for an animal can be added to a food composition of the disclosure. Examples of amino acids that can be added to a food composition of the disclosure include, but are not limited to, Arginine, Histidine, Isoleucine, Leucine, Lysine, Methionine, Methionine/Cystine, Phenylalanine, Phenylalanine/Tyrosine, Taurine, Threonine, Tryptophan, and Valine.

### Fat and Carbohydrates

For some food compositions, it may be desirable to combine the recombinant protein with fat and/or carbohydrates. Fat and carbohydrates are obtained from a variety of sources including, but not limited to, animal fat, fish oil, vegetable oil, meat, meat by-products, grains, other animal or plant sources, or any combination thereof. In some embodiments, the food product can comprise omega-3 polyunsaturated fatty acids such as docosahexaenoic acid (“DHA”) or eicosapentaenoic acid (“EPA”) or a mixture of DHA and EPA. Grains include but are not limited to rice, wheat, corn, barley, buckwheat, sorghum, oats, and quinoa. Other plant sources include but are not limited to pulses (chickpeas and different beans) and edible roots (e.g., potato, sweet potato, carrot, cassava, and turnips).

### Non-Recombinant Proteins

For some food compositions, it may be desirable to combine the recombinant protein with additional proteins (i.e., also referred to as “supplementary proteins” or “nonrecombinant proteins”). Such supplementary proteins or non-recombinant proteins, can be obtained from a variety of sources including plants, animals, or microbes (unicellular and multicellular). Supplemental proteins may also be obtained from an animal, which includes meat, meat by-products, dairy, and eggs. Meats include the flesh from poultry, fish, and animals such as cattle, swine, sheep, goats, deer, and the like. Meat by-products include but are not limited to kidneys, lungs, livers, stomachs, and intestines. In some embodiments, the supplementary proteins may be free amino acids and/or peptides.

### Fiber

For some food compositions, it may be desirable to combine the recombinant protein with fiber. Fiber can be obtained from a variety of sources such as vegetable fiber sources, including but not limited to beans, cellulose, beet pulp, parsnips, broccoli, peanut hulls, carrots, spinach, and soy fiber.

### Nutritional Supplements

For some food compositions, it may be desirable to combine the recombinant protein with nutritional supplements. The nutritional supplement can be an antioxidant, a vitamin, a mineral, or a nutrient. The nutritional supplements may be obtained from a variety of sources known to people skilled in the art including commercial sources. Vitamins and minerals can be added to a food product in amounts required to avoid deficiency and maintain health. Non-limiting examples of nutrients that can be used with the disclosure include but are not limited to choline, thiamine, egg powder, manganese, methionine, cysteine, L-carnitine, lysine, and mixtures thereof. Non-limiting examples of antioxidants include but are not limited to vitamin E, vitamin C, taurine, beta-carotene, and mixtures thereof. Vitamins generally useful as food additives include vitamin A, vitamin B 1, vitamin B2, vitamin B6, vitamin B 12, vitamin D, vitamin E, biotin, vitamin K, folic acid, inositol, pantothenic acid, niacin, pyridoxine, choline, and mixtures thereof. Minerals and trace elements useful as food additives include calcium, phosphorus, sodium, chloride, potassium, magnesium, iron, copper, zinc, selenium, iodine, and mixtures thereof. In certain embodiments, the food compositions can further comprise taurine.

### Palatability Agents

The food composition of the disclosure may comprise one or more palatability agents. The palatability agents are typically added to a food composition to enhance the overall palatability of the food to overcome any negative effects to flavor or smell. The palatability agents may be added to enhance mouth-feel or attractiveness of the food product, such as dyes or any other colorant that can change the color of the food composition. A flavoring agent may be a flavoring molecule(s) and/or flavoring precursor(s). Flavoring agents may include carbohydrates, sugars, nucleic acids (e.g., nucleotides and/or nucleosides), free fatty acids, amino acids and/or derivatives, vitamins, minerals, antioxidants, or any combination thereof. Carbohydrates and sugars may include but are not limited to, glucose, fructose, ribose, sucrose, arabinose, inositol, maltose, molasses, maltodextrin, glycogen, glycol, galactose, lactose, sorbitol, amylose, amylopectin, xylose, or any combination thereof. Nucleic acids may include but are not limited to, inosine, inosine monophosphate, guanosine, guanosine monophosphate, adenosine, adenosine monophosphate, or any combination thereof. Free fatty acids may include but are not limited to, arachidic acid, behenic acid, caprylic acid, capric acid, cerotic acid, erucic acid, auric acid, linoleic acid, myristic acid, palmitic acid, palmitoleic acid, stearic acid, lignoceric acid, or any combination thereof. Amino acids and/or amino acid derivatives may include but are not limited to, cysteine, cystine, cysteine sulfoxide, allicin, selenocystein, methionine, isoleucine, leucine, lysine, phenylalanine, threonine, tryptophan, 5-hydroxy tryptophan, valine, arginine, histidine, alanine, asparagine, aspartate, glutamate, glutamine, glycine, proline, serine, tyrosine, taurine, or any combination thereof. Amino acids may be added to the food product as free amino acids or as amino acid derivatives. For example, any amino acid may be added to the food product as a free amino acid (e.g., pre-digested amino acids without other functional groups of chemical moieties). Flavoring agents may include, but are not limited to retinol, retinal, beta-carotene, thiamine, riboflavin, niacin, niacinamide, nicotinamide, riboside, pantothenic acid, pyridoxine, pyridoxamine, pyridoxal, biotin, folates, cyanocobalamin, hydroxocobalamin, methylcobalamin, adenosylcobalamin, ascorbic acid, cholecalciferol, ergocalciferol, tocopherols (e.g., alpha-tocopherol), tocotrienols, phylloquinone, menaquinones, potassium, chlorine, sodium, calcium, phosphorus, magnesium, iron, zinc, manganese, copper, iodine, chromium, molybdenum, selenium, cobalt, or any combination thereof. Antioxidants may include, but are not limited to, beta-carotene, alpha-tocopherol, quercetin, caffeic acid, propyl gallate, epigallocatechin gallate, or any combination thereof. In some embodiments, zeolite is added to animal food compositions in amounts sufficient to enhance palatability. Preferably the amounts of zeolite that can be added to a food composition range from about 0.01% to about 4% by weight of the food composition.

### Pet Food and Feed Compositions

Various pet foods (companion animals) and animal feed (livestock, zoo animal) compositions are also provided. A pet food or animal feed composition can be made by combining a recombinant protein provided herein with a variety of other ingredients and/or additives or preservatives to generate a pet food or feed product. The one or more ingredients may be a wet ingredient, a dry ingredient, or other ingredients as provided herein, or any combination thereof. The pet food can be in various formats such as a kibble, a freeze-dried food product, a dehydrated food product, a baked food product, or raw formats.

### Food or Feed Formulations

The food or feed product can be made in various formulations. The amount of the other ingredients can be mixed with the recombinant animal protein to make the food or feed formulation will depend on the dietary requirements of a companion animal, livestock, zoo animal, which can depend on the species, age, size, weight, growth stage, health condition, and/or organ function (e.g., liver, heart, joint, hip, or brain) of the animal. In some embodiments, the pet food or feed comprising a recombinant protein is formulated to be nutritionally balanced. As used herein, the term “nutritionally balanced,” with reference to the pet food or feed composition, means that the composition has known required nutrients based on recommendations of recognized authorities in the field of pet nutrition. For example, the recommended nutrients and their amounts have been established for various animals. See, National Research Council (NRC) provides recommended amounts of such nutrients for farm animals; nutrient Requirements of Swine (11th Rev. Ed., National Academy Press, Wash. D.C., 2012); Nutrient Requirements of Poultry (9th Rev. Ed., National Academy Press, Wash. D.C., 1994); Nutrient Requirements of Horses (6th Rev. Ed., National Academy Press, Wash. D.C., 2007), each of which are incorporated in their entirety. The American Feed Control Officials (AAFCO) provides recommended amounts of such nutrients for dogs and cats. See American Feed Control Officials, Inc. (Official publication, 2018). In some embodiments, the food product comprises the AAFCO nutrient profile established for a dog. In some embodiments, the food product comprises the AAFCO nutrient profile established for a cat. In some embodiments, the feed comprises at least the minimum or the maximum nutrient concentrations as established by NRC for various farm animals, pig, sheep, chicken, horse, goat, and the like. Preferably, the food composition will include, by mass, 5-50% protein, 0.01-1.5% sodium, 0.01-1.5% potassium, 0-50% fat, 0-75% carbohydrate, 0-40% dietary fiber, and 0-15% of other nutrients. The food product comprising a recombinant protein composition can be formulated into a breed-specific food formulation. In some embodiments, the proteins for breed-specific food formulations can be based on growth rate of the breed. See for example U.S. Pat. No. 5,851,573, which is hereby incorporated by reference in its entirety. In some embodiments for breed-specific food formulations can be based on phenotypic characteristics of the animal. See for example U.S. Pat. No. 6,669,975, which is hereby incorporated by reference in its entirety. In some embodiments, the proteins for breed-specific food formulations can be based on genomic methods. See for example US Publication No. 20060045909, which is hereby incorporated by reference in its entirety. In some embodiments, the food or feed product can be formulated into a product that improves health or wellness. In some embodiments, the food or feed further comprises a compound that improves joint function, skin health, coat or hair, brain development, or improves stool quality and/or stool frequency.

### Form and Shape

The pet food or feed product (dry, semi-moist, or wet) can be in any form useful for feeding the food composition to an animal. The food product may be a shaped and/or molded or non-shaped product. For example, the food product may comprise shaped treats, kibble, edible granules, or made into a toy-shaped food product. The pet food or feed product may be formulated for mouthfeel. Mouthfeel of the pet food product may be formulated according to its structure, dryness, density, adhesiveness, bounce, chewiness, coarseness, cohesiveness, fracturability, graininess, gumminess, hardness, heaviness, moisture adsorption, moisture release, mouthcoating, roughness, slipperiness, smoothness, springiness, uniformity, and viscosity. The pet food or feed product may be formulated to have a porous, fibrous, or amorphous structure. In an example, the pet food product has a fibrous structure. The pet food product may be formulated for fracturability such that the product crumbles, cracks, or shatters. Fracturability may encompass crumbliness, crispness, crunchiness, and brittleness.

### Dry Pet Food and Feed

In some embodiments, the food product is a dry pet food or feed product for a companion animal, or dry feed for livestock, zoo animal or a pet. The dry pet food or feed product can be made completely of the recombinant animal protein. In some other embodiments, the dry pet food can comprise about 1%, 2%, 3%, 4%, 5%, 6%, 7%, 8%, 9%, 10%, 15%, 20%, 25%, 30%, 35%, 40%, 45%, 50%, 55%, 60%, 65%>, 70%, 75%, 80%, 85%, or 90% of the recombinant animal protein. A dry pet food or feed product can be prepared by adding one or more dry ingredients. Other ingredients that can be added to a dry food product include but are not limited to the ingredients provided above. The dry pet food or feed product can be freeze-dried, dehydrated, or air-dried. In some embodiments, the recombinant animal protein can be a coating on another dry food product. In some embodiments, the dry food product is a kibble. The dry pet food or feed can have the nutrient profile required for a dog or cat as provided by the AAFCO guidelines. In some embodiments, the dry feed has the nutrient profile as established by NRC for various farm animals. Kibbles are generally formed using an extrusion process in which the mixture of dry and wet ingredients is mechanically worked at high temperature and pressure and pushed through small openings and cut off into kibble by a rotating knife. Kibble also can be made using a baking process when the mix is placed into a mold before dry-heat treatment.

In some embodiments, the recombinant protein composition is coated on the dry kibble, incorporated into the kibble, or both. Other processes such as spraying, soaking, or brushing may be used to either coat the composition on the exterior or inject the recombinant protein composition into an existing dry kibble.

### Wet Pet Food and Feed

The disclosure also provides wet pet food products for a companion animal, or wet feed for livestock or a zoo animal. A wet pet food or feed can be prepared by adding one or more wet ingredients such as water containing host cells comprising recombinant protein, water, oils, fats, or vegetables or a combination thereof. Other non-limiting ingredients that can be added to a dry food product are provided above. In some embodiments, the wet food product is raw. The wet pet food or feed can be made completely of the recombinant protein. In some other embodiments, the wet pet food or feed can comprise about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, or 90% of the recombinant protein. The wet pet food or feed can have the nutrient profile required for a dog or cat as provided by the AAFCO guidelines. In some embodiments, the wet feed has the nutrient profile as established by NRC for various farm animals. The wet kibble can be a dried kibble that is coated with one or more wet topical coatings supplied as intermediate food product of the disclosure. In some embodiments, wet kibble can be made by mixing the kibble into a gravy-like liquid supplied as an intermediate food product of the disclosure.

### Pet Treats

The disclosure also provides treats for a companion animal, livestock, or a zoo animal. The treat can be a dry treat, an edible toy, or a chewable toy. The treat can be made completely of the recombinant protein. In some other embodiments, the treat can comprise about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, or 90% of the recombinant protein. Treats of the present invention can be prepared by an extrusion or baking process similar to those used for dry food. Treats of the disclosure can be prepared by a molding process. Treats can also be in the form of a chew toy. Chewable toys can include but are not limited to, artificial bones and food compositions shaped to look like natural foods that are appealing to the animal. Often, a pet treat will have nutritional value. Nutritional treats may contain one or more nutrients required for a primary food product. Non-nutritional treats can have minimal nutrition of a primary food product. Treats may also be mixed with other ingredients. Other non-limiting ingredients that can be added to a pet treat include those provided above. In some embodiments, the treat further comprises a compound that improves health or wellness. In some embodiments, the treat further comprises a compound that improves joint function, skin health, coat or hair, brain development, or improves stool quality and/or stool frequency. In some embodiments, the recombinant protein composition is coated onto the treat, incorporated into the treat, or both. Other processes such as spraying, soaking, or brushing may be used to either coat the recombinant protein as an intermediate food product composition on the exterior of the treat or inject it into an existing treat form.

### Packaging

The food compositions can be packaged in cans, trays, tubs, pouches, bags, or any other suitable container.

### Supplements

The disclosure provides supplements for a human or animal. A dietary supplement is a product intended to supplement the diet. The recombinant protein can be harvested and provided to the supplement composition as a whole-cell food composition, a protein concentrate food composition, or as a protein isolate food composition. In some embodiments, the supplement is made solely from at least one animal protein provided by the disclosure. In other embodiments, the recombinant protein is combined with other ingredients or nutrients. Other ingredients include but are not limited to those provided above. In some embodiments, a supplement can be taken by mouth. Where a supplement is formulated to be taken by mouth, it can be in the form of a pill, a capsule, a tablet, a liquid, soup, broth, or a dissolvable powder. In some embodiments, the supplement can a dry protein mixture of one or more recombinant proteins. In other embodiments, a supplement can be incorporated into a commercially available food product. In some embodiments, the recombinant animal protein is incorporated into a commercially available food product at a percentage (based on dry mass) of 0.1-95%, typically between 10% and 90%, more typically between 5% and 50%, including ranges of 5-10%, 10-20%, 20-30%, 30-40%, 40-50%, but also including 60-70%, 70-80% and 80%-90% and combinations of these ranges (e.g., 30-70%). In some embodiments, the recombinant animal protein can be incorporated into commercially available food product to increase the percentage of an essential amino acid in the product. The percentage of one or more essential amino acids can be increased in a commercially available food product by about 0.1%, 0.2%, 0.3%, 0.4%, 0.5%, 0.6%. 0.7%, 0.8%, 0.9%, 1%, 2%, 3%, 4%, 5%, 6%, 7%, 8%, 9%, 10%, 20%, 30%, 40%, 50%, 60%, 70%, 80%, or 90% (based on dry mass).

In the various forgoing food compositions and supplements, the preferred recombinant protein is a recombinant animal protein. In specific embodiments, various of the above food products and supplements include one or more recombinant annexins, FABPs, retinoid-binding proteins, or isoforms thereof or fragments thereof. In embodiments, annexins, FABPs, and/or retinoid-binding proteins useful in food compositions and supplements include those of non-human animals selected from chicken, duck, turkey, rabbit, cow, pig, sheep, tuna and salmon, among others.

### Pharmaceutical Compositions

The disclosure also provides various pharmaceutical compositions comprising a heterologous protein of the disclosure that improves the health or wellness of a human or an animal. In embodiments, the recombinant protein is a therapeutic protein as described above. In embodiments, the recombinant protein is a recombinant animal protein. These compositions can comprise, in addition to the recombinant protein, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g., oral, intravenous, cutaneous or subcutaneous, nasal, intramuscular, or intraperitoneal routes. An exemplary pharmaceutical composition can be prepared by combining a recombinant therapeutically active protein with an appropriate pharmaceutically acceptable carrier. An exemplary pharmaceutical composition can be made by combining a recombinant animal protein provided herein with a compound known or capable of improving the health or the wellness of an animal. In some embodiments, the pharmaceutical composition comprises a recombinant protein of the disclosure and a compound that improves hip function. In some embodiments, the pharmaceutical composition comprises a recombinant protein of the disclosure and a compound that improves joint function. In some embodiments, the pharmaceutical composition comprises a recombinant protein of the disclosure and a compound that improves skin health. In some embodiments, the pharmaceutical composition comprises a recombinant protein of the disclosure and a compound that improves coat or hair. In some embodiments, the pharmaceutical composition comprises a recombinant protein of the disclosure and a compound that improves brain development. In some embodiments, the pharmaceutical composition comprises a recombinant protein of the disclosure and a compound that improves stool quality and/or stool frequency. Wellness of an animal herein encompasses all aspects of the physical, mental, and social well-being of the animal, and is not restricted to the absence of infirmity. Wellness attributes include without limitation states of disease or physiological disorder, states of parasitic infestation, hair and skin condition, sensory acuteness, dispositional and behavioral attributes, and cognitive function. Conditions adverse to wellness encompass not only existing diseases and physiological including, mental, behavioral, and dispositional disorders, but also predisposition or vulnerability to such diseases or disorders. Asymptomatic conditions are likewise encompassed.

In the various forgoing pharmaceutical compositions, the preferred recombinant protein is a recombinant animal protein. In specific embodiments, various of the above pharmaceutical compositions include one or more recombinant annexins, isoforms thereof or fragments thereof. In embodiments, annexins useful in food compositions and supplements include those of non-human animals selected from chicken, deer, red deer, duck, turkey, rabbit, cow, pig, sheep, buffalo, water buffalo, tuna, cod, tilapia, and salmon, among others.

### Formulations

Pharmaceutical compositions for oral administration can be in tablet, capsule, powder or liquid form. A tablet can include a solid carrier such as gelatin or an adjuvant. In some embodiments, the capsule can be made from a vegetarian material such as agar, vegetable cellulose, and the like. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included. For intravenous, cutaneous or subcutaneous injection or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. In some embodiments, the pharmaceutical composition can be in the form of nutritional feed, a food product, or a treat.

### Methods of Treating

The disclosure also provides methods of treatment for a subject diagnosed or suffering from a disease or disorder. In embodiments, the subject is a human or any animal, and particularly a companion animal. The method can comprise administering a therapeutic-effective amount of the pharmaceutical composition provided herein alone or in combination with another agent or treatment to promote health or wellness. In some embodiments, the method includes administering a therapeutically effective amount of the pharmaceutical composition to an animal diagnosed or suffering from a disease or disorder. In yet some other embodiments, the method includes administering a prophylactically effective amount of the pharmaceutical composition to an animal genetically predisposed to a disease or a disorder. A genetically predisposed animal can be based on the breed, age, size, or any other physical characteristic. The disclosure further provides methods for making a medicament for treatment of diseases or disorders as discussed herein by combining a recombinant protein herein, particularly a recombinant therapeutic protein and/or a recombinant animal protein in a suitable pharmaceutical compositions. Additionally, the disclosure provides the use of a recombinant protein as described herein for the treatment of a disease or disorder as described herein

### Administration

For treatment purposes, the pharmaceutical composition or medicament is preferably administered to a subject in need thereof in a “therapeutically effective amount”. In some embodiments, the pharmaceutical composition is preferably administered to the subject in need thereof in “prophylactically effective amount”. The actual amount administered, and rate and time-course of administration will depend on the nature and severity of disease or disorder being treated. Prescription of treatment, e.g., decisions on dosage, etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed.), 1980. This disclosure also provides combination therapies where the pharmaceutical composition is administered in combination with another therapeutic agent or treatment. In some embodiments, the pharmaceutical composition can be administered either simultaneously or sequentially, dependent upon the condition to be treated. Non-limiting examples of a therapeutic treatment include physical therapy, surgery, radiation, and dietary restrictions for diseases, such as diabetes.

Heterologous proteins include fusion proteins. A fusion protein is a protein consisting of at least two domains that are encoded by separate genes that have been joined so that they are transcribed and translated as a single unit, producing a single polypeptide.

A gene of interest or heterologous gene can include one or more of the same or different coding sequences combined in a gene construct under the control of a single promoter and terminator. The coding sequences in such genetic constructs are optionally separated by intervening nucleotide sequences which can encode one or more flexible peptide linker or can encode a peptide sequence that is a cleavage site, e.g., a protease cleavage site or a self-cleaving peptide, or encode a combination thereof.

The term screening relates to a process of choosing one or more clones of a host organism that has been subjected to a process of transformation to introduce heterologous nucleic acid sequence to the host organism to determine if the clone of the host has been transformed and further to assess if the clone has properties associated with the presence of and expression of the heterologous nucleic acid sequence in the clone. Screening often includes selection for transformed clones on selective media (or more generally under selective growth conditions) for the presence of a selective (or selectable) marker introduced by the transformation. One of ordinary skill in the art understands the use of selective markers and choice of selective media or selective growth conditions to assess the presence or absence of the selective marker. Selective markers include expression cassettes or genes that confer antibiotic resistance. An untransformed host organism in this case is susceptible to a selected antibiotic and a selective medium containing the antibiotic is used for clone selection. Selective markers also include expression cassettes or genes which provide proteins needed to compliment auxotrophy. An untransformed host organism in this case is auxotrophic, but can be grown on media supplementing the auxotrophy. Growth of clones on medium that does not supplement the auxotrophy allows for selection of clones that are transformed with an expression cassette or gene that complements the auxotrophy. One of ordinary skill in the art, understands how to use, and apply selective growth conditions and how to use, apply and make selective media to make such selections.

Transformed host cells can be screened or further selected for the presence of and expression of one of more heterologous genes. Methods for the detection and quantitative measurement of the level or amount of a selected heterologous protein generated by a transformed host cell are known in the art, or can be readily adapted from known methods, particularly in view of disclosure provided herein. Such methods can be used to assess the level of expression of a given heterologous gene. In particular, methods are known in the art for measurement of the level of heterologous protein produced in a transformed host as a function of the total protein produced in the transformed host.

Different promoters can result in different levels of expression of a given heterologous coding sequence in a given host organism to produce heterologous protein. A promoter is stronger than another promoter if its use in an expression cassette or gene results in a higher level of production of a given heterologous protein in a given host organism. Alternatively, a promoter is weaker than another promoter if its use in an expression cassette or gene results in a lower level of production of a given heterologous protein in a given host organism. Promoter strength or weakness can depend upon the coding sequence being expressed and the host organism in which the promoter is being used to express the heterologous coding sequence.

In some embodiments, orthologs are genes in different species that evolved from a common ancestral gene by speciation, and, in general, orthologs retain the same function during the course of evolution. Isoforms are any of two or more functionally similar proteins that have a similar but not identical amino acid sequence and are either encoded by different genes or by RNA transcripts from the same gene which have had different exons removed. Annexins herein include those listed in Table 2 and isoforms and orthologs thereof.

Heterologous proteins useful in the methods and food compositions of the disclosure include those listed in Tables 3 and 4 as well as isoforms and orthologs thereof. Specifically, orthologs of the chicken proteins of Table 4 are useful as heterologous proteins in the methods and food compositions herein.

A loss of function mutation is any modification to the nucleic acid sequence of a gene that results in a significant decrease in expression of the gene or a significant decrease in function of the protein expressed from the gene. The modification can be by site mutation, or a deletion or insertion in the gene sequence.

### REFERENCES

[1] H. F. Gemede and N. Ratta, “Antinutritional factors in plant foods: Potential health benefits and adverse effects,” International Journal of Nutrition and Food Sciences, vol. 3, no. 4, pp. 284-289, 2014.
[2] G. Rimbach, J. Pallauf, J. Moehring, K. Kraemer and A. M. Minihane, “Effect of dietary phytate and microbial phyatse on mineral and traceelement bioavailability—a literature review,” Current Topics in Nutraceutical Research, vol. 6, no. 3, pp. 131-144, 2008.
[3] R. M. Yamka, U. Jamikorn, A. D. True and D. L. Harmon, “Evaluation of soyabean meal as a protein source in canine foods,” Animal Feed Science and Technology, vol. 109, pp. 121-132, 2003.
[4] K. E. Michel, “Unconventional Diets for Dogs and Cats,” Veterinary Clinics: Small Animal Practice, vol. 36, no. 6, p. 1269-1281, 2006.
[5] K. Kanakubo, A. J. Fascetti and J. A. Larsen, “Assessment of protein and amino acid concentrations and labeling adequacy of commercial vegetarian diets formulated for dogs and cats,” Journal of the American Veterinary Medical Association, vol. 247, no. 4, pp. 385-392, 2015.
[6] M. Friedman, “Nutritional Value of Proteins from Different Food Sources. A Review,” Journal of Agricultural and Food Chemistry, vol. 44, pp. 6-29, 1996.
[7] C. JVI. Gray, R. K. Sellon and L. JVI. Freeman, “Nutritional adequacy of two vegan diets for cats,” Timely Topics in Nutrition, vol. 225, no. 11, pp. 1670-1675, 2004.
[8] A. Knight and M. Leitsberger, “Vegetarian versus l\tieat-Based Diets for Companion Animals,” Animals, vol. 6, no. 57, pp. 1-20, 2016.
[9] J. L. Kaplan, J. A. Stem, A. J. Fascetti, J. A. Larsen, H. Skolnik, G. D. Peddle, R. D. Kienle, A. C. M. Waxman, C. T. Gunther-Harrington, T. Klose, K. LaFauci and B. Letbom, “Taurine deficiency and dilated cardiomyopathy in golden retrievers fed commercial diets,” PLOS ONE, 13 Dec. 2018.
[10] M. M. Rojas-Downing, A. P. Nejadhashemi, T. Harrigan and S. A. Woznicki, “Climate change and livestock: Impacts, adaptation, and mitigation,” Climate Risk Management, vol. 16, pp. 145-163, 2017.
[11] Y. M. Bar-On, R. Phillips and R. Milo, “The biomass distribution on Earth,” PNAS, vol. 115, no. 25, pp. 6506-6511, 2018.
[12] “Living Planet Report 2016,” World Wide Fund For Nature, Gland, Switzerland, 2016.
[13] F. P. J. van Bree, G. C. A. M. Bokken, R. Mineur, F. Franssen, M. Opsteegh, J. W. B. van der Giessen, L. J. A. Lipman and P. A. M. Overgaauw, “Zoonotic bacteria and parasites found in raw meat-based diets for cats and dogs,” Veterinary Record, p. 10.1136/vr.104535, 2018.
[14] A. G. Mathew, R. Cissell and S. Liamthong, “Antibiotic Resistance in Bacteria Associated with Food Animals: A United States Perspective of Livestock Production,” FOODBORNE PATHOGENS AND DISEASE, vol. 4, no. 2, pp. 115-133, 2007.
[15] S. A McEwen and P. J. Fedorka-Cray, “Antimicrobial Use and Resistance in Animals,” Clinical Infectious Diseases, vol. 34, no. Suppl 3, pp. S93-S106, 2002.
[16] W. Witte, “Selective pressure by antibiotic use in livestock,” International Journal of Antimicrobial Agents, vol. 16, pp. S19-S24, 2000.
[17] “Summary Report On Antimicrobials Sold or Distributed for Use in Food-Producing Animals,” Food and Drug Administration, 2014.
[18] J. E. Markovich, C. R. Heinze and L. M. Freeman, “Thiamine deficiency in dogs and cats,” Journal of the American Veterinary Medical Association, vol. 243, no. 5, pp. 649-656, 2013.
[19] M. Steer, “A Comparison Of Land, Water And Energy Use Between Conventional And Yeast-Derived Dairy Products: An Initial Analysis,” University of the West of England, 2015.
[20] X. Zheng, K. Diraviyam and D. Sept, “Nucleotide Effects on the Structure and Dynamics of Actin,” Biophysical Journal, vol. 93, no. 4, p. 1277-1283, 2007. Actin Surface,” in Actin-Monomer-Binding Proteins, New York, NY: Springer, 2007.
[22] T. D. Pollard and G. G. Borisy, “Cellular Motility Driven by Assembly and Disassembly of Actin Filaments,” Cell, vol. 112, no. 4, pp. 453-465, 2003. 10.
[23] B. Peng et al. (2022) “An in vivo gene amplification system for high level expression in Saccharomyces cerevisiae,” Nature Communications, vol. 13:2895.
[24] Cleary M A. Haploinsufficiency, Encyclopedia of Genetics (2001)
[25] Ohnuki S, and Ohya Y. High-dimensional single-cell phenotyping reveals extensive haploinsufficiency. PLoS Biology (2018)
[26] Deutschbauer A M, Jaramillo D F, Proctor M, Kumm J, Hillenmeyer M E, Davis R W, et al. Mechanisms of haploinsufficiency revealed by genome-wide profiling in yeast. Genetics (2005)
[27] Delneri D, Hoyle D C, Gkargkas K, Cross E J, Rash B, Zeef L, et al. Identification and characterization of high-flux-control genes of yeast through competition analyses in continuous cultures. Nature Genetics (2008)
[28] Schimke R T. Gene amplification in cultured animal-cells. Cell (1984)
[29] Sauer B. Functional expression of the cre-lox site-specific recombination system in the yeast Saccharomyces cerevisiae. Mol Cell Biol. (1987).
[30] Pir P., et al. The genetic control of growth rate: a systems biology study in yeast. BMC Syst Biol (2012)
[31] Novo et al. Genome-wide study of the adaptation of Saccharomyces cerevisiae to the early stages of wine fermentation. PloS One 2013 5; 8(9):e74086.
[32] Shimada et al. Genome-wide study of the adaptation of Saccharomyces cerevisiae to the early stages of wine fermentation. Mol. Cell. 2013 26; 51(6):829-839.

### Aspects of the Invention

Various aspects are contemplated herein, several of which are set forth in the paragraphs below. It is explicitly contemplated that any aspect or portion thereof can be combined to form an aspect. In addition, it is explicitly contemplated that any aspect (e.g., Aspect 13) that references an aspect (e.g., Aspect 1) for which there are sub-aspects having the same top level number (e.g., Aspect 1A, 1B, 1C, and so forth) necessarily includes reference to those sub-aspects 1A, 1B, 1C, and so forth. Furthermore, it is explicitly contemplated that aspects can be combined in any manner. Moreover, the term “any preceding aspect” means any aspect that appears prior to the aspect that contains such phrase (in other words, the sentence “Aspect 100: The method of any one of aspects 50-99, or any preceding aspect, . . . ” means that any aspect prior to aspect 100 is referenced, including aspects 1-49). For example, it is contemplated that, optionally, any method or composition of any of the below aspects may be useful with or combined with any other aspect provided below. Further, for example, it is contemplated that any embodiment described elsewhere herein, including above this paragraph, may optionally be combined with any of the below listed aspects. In some instances in the aspects below, or elsewhere herein, two open ended ranges are disclosed to be combinable into a range. For example, “at least X” is disclosed to be combinable with “less than Y” to form a range, in which X and Y are numeric values. For the purposes of forming ranges herein, it is explicitly contemplated that “at least X” combined with “less than Y” forms a range of X-Y inclusive of value X and value Y, even though “less than Y” in isolation does not include Y.

Aspect 1. A method for making heterologous polypeptides of interest in fungi for use in food compositions, the method comprising:
introducing one or more genetic constructs comprising a gene of interest encoding a heterologous annexin-like protein (ALP) into the genome of a fungus to generate one or more transformed fungal strains;
wherein the one or more transformed fungi strains comprise an ALP content of at least 2% (e.g., at least 2%, at least 3%, at least 4%, at least 5%, at least 6%, at least 7%, at least 8%, at least 9%, at least 10%, at least 15%, at least 20%, at least 30%, optionally less than 100%, less than 95%, less than 90%, less than 85%, or less than 75%) of the total protein content of the transformed fungal strains;
thereby making heterologous polypeptides of interest in fungi for use in food compositions.

Aspect 1A. A method for making heterologous polypeptides of interest in fungi for use in food compositions, the method comprising:
expressing in a fungi one or more genetic constructs comprising a gene of interest encoding a heterologous annexin-like protein (ALP) into the genome of a fungus to generate one or more transformed fungal strains;
wherein the one or more transformed fungi strains comprise an ALP content of at least 2% (e.g., at least 2%, at least 3%, at least 4%, at least 5%, at least 6%, at least 7%, at least 8%, at least 9%, at least 10%, at least 15%, at least 20%, at least 30%, optionally less than 100%, less than 95%, less than 90%, less than 85%, or less than 75%) of the total protein content of the transformed fungal strains;
thereby making heterologous polypeptides of interest in fungi for use in food compositions.

Aspect 1B. A method for making heterologous polypeptides of interest in fungi for use in food compositions, the method comprising:
culturing a fungi comprising one or more genetic constructs comprising a gene of interest encoding a heterologous annexin-like protein (ALP) into the genome of a fungus to generate one or more transformed fungal strains;
wherein the one or more transformed fungi strains comprise an ALP content of at least 2% (e.g., at least 2%, at least 3%, at least 4%, at least 5%, at least 6%, at least 7%, at least 8%, at least 9%, at least 10%, at least 15%, at least 20%, at least 30%, optionally less than 100%, less than 95%, less than 90%, less than 85%, or less than 75%) of the total protein content of the transformed fungal strains;
thereby making heterologous polypeptides of interest in fungi for use in food compositions.

Aspect 2. A method for generating transformed yeast strains capable of enhanced expression of one or more heterologous polypeptides of interest, the method comprising:
providing at least one genetic construct comprising:
   a first promoter homologous to at least a portion of the native promoter region of a haploinsufficient gene tied to yeast fitness;
   an auxotrophic marker;
   a gene of interest encoding a heterologous polypeptide of interest; and
   a synthetic open reading frame (ORF) homologous to at least a portion of a native ORF of the haploinsufficient gene tied to yeast fitness;
   integrating the at least one genetic construct into the haploinsufficient gene tied to yeast fitness to produce one or more transformed yeast strains, wherein the integrating generates a tandem amplification region; and
   screening the one or more transformed yeast strains based on (i) auxotrophy; (ii) yeast fitness; (iii) total protein content, and/or (iv) heterologous polypeptide of interest content and an gene copy number of the coding sequence of the heterologous polypeptide of interest;
   selecting the transformed yeast strains having an heterologous polypeptide of interest content of at least 2% (e.g., at least 2%, at least 3%, at least 4%, at least 5%, at least 6%, at least 7%, at least 8%, at least 9%, at least 10%, at least 15%, at least 20%, at least 30%, optionally less than 100%, less than 95%, less than 90%, less than 85%, or less than 75%) of the total protein content;
thereby generating transformed yeast strains capable of enhanced expression of one or more heterologous polypeptides of interest.

Aspect 3. The method of any of the preceding Aspects, wherein the at least one genetic construct comprises, from 5′ to 3′:
the first promoter;
the auxotrophic marker;
the gene of interest; and
the synthetic ORF.

Aspect 4. The method of any of the preceding Aspects, wherein the at least one genetic construct comprises, from 5′ to 3′:
the first promoter;
the auxotrophic marker;
a synthetic terminating region of the haploinsufficient gene tied to yeast fitness;
the gene of interest;
a second promoter; and
the synthetic ORF.

Aspect 5. The method of any of the preceding Aspects, wherein the at least one genetic construct comprises, from 5′ to 3′:
the first promoter;
the auxotrophic marker;
a synthetic terminating region of the haploinsufficient gene tied to yeast fitness;
the gene of interest;
an autonomous replicating sequence (ARS);
a second promoter; and
the synthetic ORF.

Aspect 6. The method of Aspects 4 or 5 or any preceding Aspect, wherein the second promoter is operably connected to the synthetic ORF and the native terminating region of the haploinsufficient gene.

Aspect 7. The method of any one of Aspects 4-6 or any preceding Aspect, wherein the second promoter comprises a DNA sequence different from the sequence of the native promoter region of the haploinsufficient gene tied to yeast fitness.

Aspect 8. The method of any one of Aspects 4-7 or any preceding Aspect, wherein the second promoter is weaker than the first promoter.

Aspect 9. The method of any one of Aspects 2-8 or any preceding Aspect, wherein the tandem amplification region comprises the gene of interest and the synthetic ORF.

Aspect 10. The method of any one of Aspects 2-9 or any preceding Aspect, wherein the tandem amplification region comprises:
a synthetic terminating region of the haploinsufficient gene;
the gene of interest;
a second promoter;
the synthetic ORF; and
the native terminating region of the haploinsufficient gene.

Aspect 11. The method of Aspect 10 or any preceding Aspect, wherein the tandem amplification region further comprises an autonomous replicating sequence (ARS).

Aspect 12. The method of Aspect 11 or any preceding Aspect, wherein the tandem amplification region comprises, from 5′ to 3′:
the synthetic terminating region of the haploinsufficient gene;
the ARS;
the gene of interest;
the second promoter;
the synthetic ORF; and
the native terminating region of the haploinsufficient gene.

Aspect 13. The method of any one of Aspects 2-12 or any preceding Aspect, wherein the method comprises providing at least two genetic constructs.

Aspect 14. The method of Aspect 13 or any preceding Aspect, wherein the at least two genetic constructs are integrated into at least two distinct haploinsufficient genes tied to yeast fitness.

Aspect 15. The method of any one of Aspects 2-14 or any preceding Aspect, wherein the at least one genetic construct comprises two or more genes of interest.

Aspect 16. The method of any one of Aspects 2-15 or any preceding Aspect, wherein the auxotrophic marker is URA3, LEU2, HIS3, TRP1, LYS2, ADE2, or MET3.

Aspect 17. The method of any one of Aspects 2-16 or any preceding Aspect, wherein the auxotrophic marker is URA3.

Aspect 18. The method of any one of Aspects 2-17 or any preceding Aspect, wherein the auxotrophic marker is a recyclable marker.

Aspect 19. The method of any one of Aspects 2-18 or any preceding Aspect, wherein the haploinsufficient gene tied to yeast fitness is a gene encoding a protein selected from Table 6.

Aspect 20. The method of any one of Aspects 2-19 or any preceding Aspect, wherein the haploinsufficient gene tied to yeast fitness is selected from a gene encoding a transcriptional protein, a copper resistance protein, a coat protein complex, or a ribosomal subunit protein.

Aspect 21. The method of any one of Aspects 2-20 or any preceding Aspect, wherein the haploinsufficient gene tied to yeast fitness is a gene encoding for a ribosomal subunit protein.

Aspect 22. The method of Aspect 21 or any preceding Aspect, wherein the ribosomal subunit protein is RPL25, RPL33a, RPL17a, RPN11, or RPB7.

Aspect 23. The method of claim 22 or any preceding Aspect, wherein the ribosomal subunit protein is RPL25.

Aspect 24. The method of any preceding Aspect, wherein the gene of interest encodes a heterologous ALP of interest or a heterologous FLP of interest.

Aspect 25. The method of Aspect 24 or any preceding Aspect, wherein the heterologous ALP of interest comprises an annexin repeat sequence having between 60 and 80 amino acids (e.g., between 60 and 80 amino acids, between 60 and 75 amino acids, or between 60 and 70 amino acids, or any subrange thereof).

Aspect 26. The method of Aspect 24 or 25 or any preceding Aspect, wherein the heterologous ALP comprises a leader sequence having between 25 and 40 amino acids (e.g., between 25 and 35 amino acids or between 25 and 30 amino acids, or any subrange thereof).

Aspect 27. The method of any one of Aspects 24-26 or any preceding Aspect, wherein a portion of the heterologous ALP is characterized by a tightly packed structure containing one or more alpha helices.

Aspect 28. The method of any one of Aspects 24-27 or any preceding Aspect, wherein the heterologous ALP is characterized by a median molecular weight of between 20 kDa and 80 kDa (e.g., between 25 kDa and 75 kDa, between 30 kDa and 70 kDa, between 35 kDa and 65 kDa, between 40 kDa and 60 kDa, between 45 kDa and 55 kDa, or any subrange thereof).

Aspect 29. The method of any one of Aspects 24-28 or any preceding Aspect, wherein the heterologous ALP is characterized by a median molecular weight of between 35 kDa and 55 kDa (e.g., between 40 kDa and 50 kDa, or any subrange thereof).

Aspect 30. The method of any one of Aspects 24-29 or any preceding Aspect, wherein the heterologous ALP is a calcium-dependent membrane-binding protein.

Aspect 31. The method of any one of Aspects 24-30 or any preceding Aspect, wherein the heterologous ALP is an annexin.

Aspect 32. The method of any one of Aspects 24-31 or any preceding Aspect, wherein the heterologous ALP is an annexin selected from the group consisting of annexin A1, annexin A2, annexin A3, annexin A4, annexin A5, annexin A6, annexin A7, annexin A8, annexin A9, annexin A10, annexin A11, annexin A13, and any isoforms thereof.

Aspect 33. The method of Aspect 32 or any preceding Aspect, wherein the heterologous ALP is annexin A3.

Aspect 34. The method of any one of Aspects 24-31 or any preceding Aspect, wherein the heterologous ALP is an annexin selected from the group consisting of annexin B9, annexin B10, annexin B11, annexin B12, annexin C1, annexin C2, annexin C3, annexin C4, annexin C5, any one of annexins D1-D25, annexin E1, annexin E2, annexin E3 and any isoforms thereof.

Aspect 35. The method of any one of Aspects 2-34 or any preceding Aspect, wherein the gene of interest is an ortholog functionally analogous to a selected native gene of interest.

Aspect 36. The method of Aspect 35 or any preceding Aspect, further comprising:
deleting the CDS of the selected native gene of interest from a deletion site of the yeast genome;
wherein the deleting step is performed prior to, or simultaneously with, the integrating step.

Aspect 37. The method of Aspect 35 or 36 or any preceding Aspect, wherein the ortholog comprises a CDS having a distinct, or substantially different, DNA sequence of the native CDS of the haploinsufficient gene tied to yeast fitness.

Aspect 38. The method of any one of Aspects 35-37 or any preceding Aspect, wherein the deleting step comprises homologous recombination with a selectable marker.

Aspect 39. The method of Aspect 38 or any preceding Aspect, wherein the selectable marker is an auxotrophic marker, an antibiotic resistance marker, a dominant drug marker, or a counter-selectable marker.

Aspect 40. The method of Aspect 38 or 39 or any preceding Aspect, wherein the selectable marker is a recyclable antibiotic resistance marker.

Aspect 41. The method of Aspect 40 or any preceding Aspect, wherein the recyclable antibiotic resistance marker is KanMX, NatMX, HphMX, or Ble.

Aspect 42. The method of any one of Aspects 33-40 or any preceding Aspect, wherein the deletion site is a site different from the site of the haploinsufficient gene tied to yeast fitness.

Aspect 43. A method for generating a transformed yeast strain capable of enhanced expression of one or more heterologous polypeptides of interest, the method comprising:
providing at least one modified genetic construct comprising:
a first promoter homologous to at least a portion of the native promoter region of a haploinsufficient gene tied to yeast fitness;
a selectable marker;
an ortholog of interest encoding a heterologous polypeptide of interest;
a modified open reading frame (ORF) having a reduced expression level or activity level as compared to the native ORF of the haploinsufficient gene tied to yeast fitness due to a loss-of-function mutation;
integrating the modified genetic construct into the haploinsufficient gene tied to yeast fitness to produce one or more transformed yeast strains, wherein the integrating generates a tandem amplification region; and
screening the one or more transformed yeast strains based on (i) the presence of the selectable marker; (ii) yeast fitness; (iii) total protein content, and (iv) heterologous polypeptide of interest content and a gene copy number of the coding sequence of the heterologous polypeptide of interest;
thereby generating yeast strains capable of enhanced expression of a heterologous polypeptide of interest.

Aspect 44. The method of Aspect 43 or any preceding Aspect, further comprising: selecting the transformed yeast strains having a heterologous polypeptide of interest content of at least 2% (e.g., at least 2%, at least 3%, at least 4%, at least 5%, at least 6%, at least 7%, at least 8%, at least 9%, at least 10%, at least 15%, at least 20%, at least 30%, optionally less than 100%, less than 95%, less than 90%, less than 85%, or less than 75%) of the total protein content.

Aspect 45. The method of Aspect 43 or 44 or any preceding Aspect, wherein the at least one modified genetic construct comprises, from 5′ to 3′:
the first promoter;
the selectable marker;
the ortholog of interest; and
the modified ORF.

Aspect 46. The method of any one of Aspects 43-45 or any preceding Aspect, wherein the at least one modified genetic construct comprises, from 5′ to 3′:
the first promoter;
the selectable marker;
a synthetic terminating region of the haploinsufficient gene tied to yeast fitness;
the ortholog of interest;
a second promoter; and
the modified ORF.

Aspect 47. The method of any one of Aspects 43-46 or any preceding Aspect, wherein the at least one modified genetic construct comprises, from 5′ to 3′:
the first promoter;
the selectable marker;
a synthetic terminating region of the haploinsufficient gene tied to yeast fitness;
the ortholog of interest;
an autonomous replicating sequence (ARS);
a second promoter; and
the modified ORF.

Aspect 48. The method of Aspect 46 or 47 or any preceding Aspect, wherein the second promoter is operably connected to the modified ORF and the native terminating region of the haploinsufficient gene.

Aspect 49. The method of any one of Aspects 46-48 or any preceding Aspect, wherein the second promoter comprises a DNA sequence different from the sequence of the native promoter region of the haploinsufficient gene tied to yeast fitness.

Aspect 50. The method of any one of Aspects 46-49 or any preceding Aspect, wherein the second promoter is weaker than the first promoter.

Aspect 51. The method of any one of Aspects 43-50 or any preceding Aspect, wherein the tandem amplification region comprises the ortholog of interest and the modified ORF.

Aspect 52. The method of any one of Aspects 43-51 or any preceding Aspect, wherein the tandem amplification region comprises:
a synthetic terminating region of the haploinsufficient gene;
the ortholog of interest;
a second promoter;
the modified ORF; and
the native terminating region of the haploinsufficient gene.

Aspect 53. The method of Aspect 52 or any preceding Aspect, wherein the tandem amplification region further comprises an autonomous replicating sequence (ARS).

Aspect 54. The method of Aspect 53 or any preceding Aspect, wherein the tandem amplification region comprises, from 5′ to 3′:
the synthetic terminating region of the haploinsufficient gene;
the ARS;
the ortholog of interest;
the second promoter;
the modified ORF; and
the native terminating region of the haploinsufficient gene.

Aspect 55. The method of any one of Aspects 43-54 or any preceding Aspect, wherein the method comprises providing at least two modified genetic constructs (e.g., at least two, at least three, at least four, at least five, at least ten, at least fifteen, optionally less than one-hundred modified genetic constructs).

Aspect 56. The method of Aspect 55 or any preceding Aspect, wherein the at least two genetic constructs are integrated into at least two distinct haploinsufficient genes tied to yeast fitness (e.g., at least two, at least three, at least four, at least five, at least ten, at least fifteen, optionally less than one-hundred distinct haploinsufficient genes tied to yeast fitness).

Aspect 57. The method of any one of Aspects 43-56 or any preceding Aspect, wherein the at least one genetic construct comprises two or more genes of interest (e.g., two or more, three or more, four or more, five or more, ten or more, fifteen or more, twenty or more, thirty or more, fifty or more, one-hundred or more, optionally less than one-thousand, less than five-hundred, less than one-hundred genes of interest).

Aspect 58. The genetic construct of any one of Aspects 43-57 or any preceding Aspect, wherein the selectable marker is an auxotrophic marker, an antibiotic resistance marker, a dominant drug marker, or a counter-selectable marker.

Aspect 59. The genetic construct of any one of Aspects 43-58 or any preceding Aspect, wherein the selectable marker is an auxotrophic marker.

Aspect 60. The genetic construct of any one of Aspects 43-59 or any preceding Aspect, wherein the selectable marker is a recyclable marker.

Aspect 61. The method of any one of Aspects 43-60 or any preceding Aspect, wherein the selectable marker is URA3, LEU2, HIS3, TRP1, LYS2, ADE2, or MET3.

Aspect 62. The method of any one of Aspects 43-61 or any preceding Aspect, wherein the selectable marker is URA3.

Aspect 63. The method of any one of Aspects 35-62 or any preceding Aspect, wherein the ortholog encodes a protein involved in one or more of protein metabolism, transcription from RNA polymerase II, proteasome functionality, DNA replication, translation initiation, transcriptional regulation, endoplasmic reticulum-to-Golgi apparatus transport, nuclear import, nuclear export, cytoskeletal function, and glycolysis.

Aspect 64. The method of Aspect 63 or any preceding Aspect, wherein the ortholog of interest encodes a protein involved in glycolysis.

Aspect 65. The method of Aspect 64 or any preceding Aspect, wherein the protein involved in glycolysis is TDH3, HXK1, GPM1, ENO1, TPI1, CDC19, PDC2, or ADH3, or any isoform thereof.

Aspect 66. The method of Aspect 65 or any preceding Aspect, wherein the protein is TDH3, or any isoform thereof.

Aspect 67. The method of any one of Aspects 43-66 or any preceding Aspect, wherein the loss-of-function mutation comprises a nonsense mutation, a missense mutation, a point mutation, or a frameshift mutation.

Aspect 68. The method of any one of Aspects 43-67 or any preceding Aspect, wherein the loss-of-function mutation comprises a nonsense mutation.

Aspect 69. The method of any one of Aspects 43-68 or any preceding Aspect, wherein the ortholog of interest is functionally analogous to the haploinsufficient gene tied to yeast fitness.

Aspect 70. The method of any one of Aspects 43-69 or any preceding Aspect, wherein the haploinsufficient gene tied to yeast fitness is a gene encoding a protein selected from Table 6.

Aspect 71. The method of any one of Aspects 43-70 or any preceding Aspect, wherein the haploinsufficient gene tied to yeast fitness is selected from a gene encoding for a transcriptional protein, a copper resistance protein, a coat protein complex, or a ribosomal subunit protein.

Aspect 72. The method of any one of Aspects 43-71 or any preceding Aspect, wherein the haploinsufficient gene tied to yeast fitness is a gene encoding for a ribosomal subunit protein.

Aspect 73. The method of Aspect 72 or any preceding Aspect, wherein the ribosomal subunit protein is RPL25, RPL33a, or RPL17a.

Aspect 74. The method of Aspect 73 or any preceding Aspect, wherein the ribosomal subunit protein is RPL25.

Aspect 75. The method of any one of Aspects 43-74 or any preceding Aspect, wherein the gene of interest encodes a heterologous ALP of interest or a heterologous FLP of interest.

Aspect 76. The method of Aspect 75 or any preceding Aspect, wherein the heterologous ALP of interest comprises an annexin repeat sequence having between 60 and 80 amino acids (e.g., between 65 and 75 amino acids, or any subrange thereof).

Aspect 77. The method of Aspect 75 or 76 or any preceding Aspect, wherein the heterologous ALP comprises a leader sequence having between 25 and 40 amino acids (e.g., between 30 and 35 amino acids, or any subrange thereof).

Aspect 78. The method of any one of Aspects 75-77 or any preceding Aspect, wherein a portion of the heterologous ALP is characterized by a tightly packed structure containing one or more alpha helices.

Aspect 79. The method of any one of Aspects 75-78 or any preceding Aspect, wherein the heterologous ALP is characterized by an average molecular weight of between 20 kDa and 80 kDa (e.g., between 25 kDa and 75 kDa, between 30 kDa and 70 kDa, between 35 kDa and 65 kDa, between 40 kDa and 60 kDa, between 45 kDa and 55 kDa, or any subrange thereof).

Aspect 80. The method of any one of Aspects 75-79 or any preceding Aspect, wherein the heterologous ALP is characterized by an average molecular weight of between 35 kDa and 55 kDa (e.g., between 40 kDa and 50 kDa, or any subrange thereof).

Aspect 81. The method of any one of Aspects 75-80 or any preceding Aspect, wherein the heterologous ALP is a calcium-dependent membrane-binding protein.

Aspect 82. The method of any one of Aspects 75-81 or any preceding Aspect, wherein the heterologous ALP is an annexin.

Aspect 83. The method of any one of Aspects 75-82 or any preceding Aspect, wherein the heterologous ALP is an annexin selected from the group consisting of annexin A1, annexin A2, annexin A3, annexin A4, annexin A5, annexin A6, annexin A7, annexin A8, annexin A9, annexin A10, annexin A11, annexin A13, and any isoforms thereof.

Aspect 84. The method of any one of Aspects 75-82 or any preceding Aspect, wherein the heterologous ALP is an annexin selected from the group consisting of annexin B9, annexin B10, annexin B11, annexin B12, annexin C1, annexin C2, annexin C3, annexin C4, annexin C5, any one of annexins D1-D25, annexin E1, annexin E2, annexin E3 and any isoforms thereof.

Aspect 85. The method of any one of the preceding Aspects, wherein the heterologous polypeptide of interest is a heterologous protein.

Aspect 86. The method of Aspect 85 or any preceding Aspect, wherein the heterologous protein is characterized by an average molecular weight of between 10 kDa and 250 kDa (e.g., between 15 kDa and 200 kDa, between 20 kDa and 150 kDa, between 25 kDa and 100 kDa, between 50 kDa and 75 kDa, or any subrange thereof).

Aspect 87. The method of Aspect 85 or any preceding Aspect, wherein the heterologous protein is characterized by an average molecular weight of between 10 kDa and 100 kDa (e.g., between 15 kDa and 90 kDa, between 20 kDa and 80 kDa, between 25 kDa and 75 kDa, between 35 kDa and 65 kDa, or any subrange thereof).

Aspect 88. The method of Aspect 85 or any preceding Aspect, wherein the heterologous protein is characterized by an average molecular weight of between 20 kDa and 100 kDa (e.g., between 25 kDa and 95 kDa, between 30 kDa and 90 kDa, between 35 kDa and 85 kDa, between 40 kDa and 80 kDa, between 45 kDa and 75 kDa, between 50 kDa and 70 kDa, between 55 kDa and 65 kDa, or any subrange thereof).

Aspect 89. The method of Aspect 85 or any preceding Aspect, wherein the heterologous protein is characterized by an average molecular weight of between 20 kDa and 80 kDa (e.g., between 25 kDa and 75 kDa, between 30 kDa and 70 kDa, between 35 kDa and 65 kDa, between 40 kDa and 60 kDa, between 45 kDa and 55 kDa, or any subrange thereof).

Aspect 90. The method of Aspect 85 or any preceding Aspect, wherein the heterologous protein is a cytoskeletal protein.

Aspect 91. The method of Aspect 85 or 90 or any preceding Aspect, wherein the heterologous protein is an actin cytoskeletal protein.

Aspect 92. The method of Aspect 91 or any preceding Aspect, wherein the actin cytoskeletal protein is selected from the group consisting of: an actin, an actin-binding protein, an actin-bundling protein, a monomer binding protein, a cytoskeletal linker protein, a membrane anchor protein, a stabilizing protein, a signaling protein, a capping protein, a severing protein, or a myosin.

Aspect 93. The method of Aspect 85 or any preceding Aspect, wherein the heterologous protein is a membrane-binding protein.

Aspect 94. The method of Aspect 85 or any preceding Aspect, wherein the heterologous protein is a calcium-dependent membrane-binding protein.

Aspect 95. The method of Aspect 94 or any preceding Aspect, wherein the calcium-dependent membrane-binding protein is an annexin.

Aspect 96. The method of Aspect 85 or any preceding Aspect, wherein the heterologous protein is a protein selected from Table 1, or an isoform thereof.

Aspect 97. The method of Aspect 85 or any preceding Aspect, wherein the heterologous protein is a protein selected from Table 2, or an isoform thereof.

Aspect 98. The method of Aspect 85 or any preceding Aspect, wherein the heterologous protein is a protein selected from Table 3, or an isoform thereof.

Aspect 99. The method of any one of Aspects 85-98 or any preceding Aspect, wherein the heterologous protein is a protein intended for use in a food product.

Aspect 100. The method of any one of the preceding Aspects, wherein the heterologous polypeptide of interest is at least a portion of a vertebrate animal protein.

Aspect 101. The method of Aspect 100 or any preceding Aspect, wherein the vertebrate animal is a pig, a turkey, a chicken, a pheasant, a quail, a horse, a cow, a fish, a sheep, a deer, a red deer, a duck, a rabbit, an elk, a moose, a kangaroo, an alligator, a lamb, a wild boar, a goat, a bison, a water buffalo, or a buffalo.

Aspect 102. The method of Aspect 100 or 101 or any preceding Aspect, wherein the vertebrate animal protein is a muscle protein.

Aspect 103. The method of any one of the preceding Aspects, wherein the heterologous polypeptide of interest has an amino acid sequence that is modified relative to the naturally occurring sequence.

Aspect 104. The method of any one of the preceding Aspects, wherein the heterologous polypeptide of interest is codon-optimized for expression in fungi.

Aspect 105. The method of any one of the preceding Aspects, wherein the heterologous polypeptide of interest is codon-optimized for expression in yeast.

Aspect 106. The method of Aspect 1, wherein the fungi is yeast.

Aspect 107. The method of any one of Aspects 2-106 or any preceding Aspect, wherein the yeast is a budding yeast.

Aspect 108. The method of any one of Aspects 2-107 or any preceding Aspect, wherein the yeast is of the Saccharomyces genus.

Aspect 109. The method of Aspect 108 or any preceding Aspect, wherein the yeast is selected from the group consisting of: Saccharomyces cerevisiae, Saccharomyces uvarum, Saccharomyces bayanus, and Saccharomyces paradoxus.

Aspect 110. The method of Aspect 109 or any preceding Aspect, wherein the yeast is Saccharomyces cerevisiae.

Aspect 111. The method of any one of Aspects 2-41 or 84-111 or any preceding Aspect, wherein the screening the one or more transformed yeast strains is performed sequentially as follows:
conducting a first screen to select for at least one of the one or more transformed yeast strains depicting the presence of the auxotrophic marker;
conducting a second screen on the at least one transformed yeast strains based on yeast fitness;
conducting a third screen based on heterologous polypeptide of interest content and the gene copy number of the CDS of the heterologous polypeptide of interest;
conducting a fourth screen based on total protein content.

Aspect 112. The method of any one of Aspects 43-110 or any preceding Aspect, wherein the screening the one or more transformed yeast strains is performed sequentially as follows:
conducting a first screen to select for at least one of the one or more transformed yeast strains depicting the presence of the selectable marker;
conducting a second screen on the at least one transformed yeast strains based on yeast fitness;
conducting a third screen based on heterologous polypeptide of interest content and the gene copy number of the CDS of the heterologous polypeptide of interest;
conducting a fourth screen based on total protein content.

Aspect 113. The method of Aspect 111 or 112 or any preceding Aspect, wherein the gene copy number is between 2 and 1000 gene copies, such as between 2 and 500 copies, 2 and 250 copies, 2 and 100 copies, 2 and 50 gene copies, or 2 and 40 copies.

Aspect 114. The method of any one of Aspects 111-113 or any preceding Aspect, wherein the second screen comprises:
comparing a yeast growth percentage of the one or more transformed yeast strains to a yeast growth percentage of non-transformed yeast;
wherein the yeast growth percentage of the one or more transformed yeast is within 30% of the yeast growth percentage of the non-transformed yeast.

Aspect 115. The method of Aspect 114 or any preceding Aspect, wherein the yeast growth percentage of the one or more transformed yeast is within 40% (e.g., within 40%, within 35%, within 30%, within 25%, within 20%, or within 15%) of the yeast growth percentage of the non-transformed yeast.

Aspect 116. The method of Aspect 114 or any preceding Aspect, wherein the yeast growth percentage of the one or more transformed yeast is within 15% (e.g., within 12%, within 10%, within 7%, within 5%, within 4%, within 3%, within 2%, or within 1%) of the yeast growth percentage of the non-transformed yeast.

Aspect 117. The method of any one of Aspects 2-116 or any preceding Aspect, wherein the screening the one or more transformed yeast strains further comprises:
growing the one or more transformed yeast strains using selective growth media to produce transformed yeast colonies;
selecting at least one colony of the transformed yeast colonies, wherein the at least one colony is characterized by a diameter which is at least 20% (e.g., at least 20%, at least 30%, at least 40%) smaller than the average colony diameter of the transformed yeast colonies.

Aspect 118. The method of Aspect 117 or any preceding Aspect, wherein the at least one colony is characterized by a diameter which is at least 50% (e.g., at least 50%, at least 60%, at least 70%) smaller than the average colony diameter of the transformed yeast colonies.

Aspect 119. The method of Aspect 117 or any preceding Aspect, wherein the at least one colony is characterized by a diameter which is at least 75% (e.g., at least 75%, at least 85%) smaller than the average colony diameter of the transformed yeast colonies.

Aspect 120. The method of Aspect 117 or any preceding Aspect, wherein the at least one colony is characterized by a diameter which is at least 90% (e.g., at least 90%, at least 95%) smaller than the average colony diameter of the transformed yeast colonies.

Aspect 121. The method of any one of Aspects 117-120 or any preceding Aspect, further comprising:
growing the at least one colony using the selective growth media to produce at least one master colony;
suspending the at least one master colony in a lysis buffer to form a master colony solution; and
selecting the master colony solution based on a color, wherein the color is visually pink.

Aspects 122-126. reserved.

Aspect 127. The method of any one of the preceding Aspects, further comprising selecting the transformed yeast strains having an heterologous polypeptide of interest content of at least 3% of the total protein content.

Aspect 128. The method of any one of the preceding Aspects, further comprising selecting the transformed yeast strains having an heterologous polypeptide of interest content of at least 4% of the total protein content.

Aspect 129. The method of any one of the preceding Aspects, further comprising selecting the transformed yeast strains having a heterologous polypeptide of interest content of at least 5% of the total protein content.

Aspect 130. The method of any one of the preceding Aspects, further comprising selecting the transformed yeast strains having a heterologous polypeptide of interest content of at least 10% of the total protein content.

Aspect 131. The method of any one of the preceding Aspects, further comprising selecting the transformed yeast strains having a heterologous polypeptide of interest content of at least 15% (e.g., at least 15%, at least 20%, at least 25%, at least 30%, at least 35%, at least 40%, at least 45%) of the total protein content.

Aspect 132. The method of any one of Aspects 2-131 or any preceding Aspect, wherein the method increases the total protein content of the transformed yeast strains by 2% or more based on dry weight as compared to the total protein content of a wild-type yeast of the same species.

Aspect 133. The method of any one of the preceding Aspects 2-131 or any preceding Aspect, wherein the method increases the total protein content of the transformed yeast strains by 3% or more based on dry weight as compared to the total protein content of a wild-type yeast of the same species.

Aspect 134. The method of any one of the preceding Aspects 2-131 or any preceding Aspect, wherein the method increases the total protein content of the transformed yeast strains by 4% or more based on dry weight as compared to the total protein content of a wild-type yeast of the same species.

Aspect 135. The method of any one of the preceding Aspects 2-131 or any preceding Aspect, wherein the method increases the a total protein content of the transformed yeast strains by 5% or more based on dry weight as compared to the total protein content of a wild-type yeast of the same species.

Aspect 136. A transformed fungal strain comprising at least one of the genetic constructs of any one of the preceding Aspects 1-134 or any preceding Aspect.

Aspect 137. A food composition comprising one or more transformed fungal strains of Aspect 136 or any preceding Aspect.

Aspect 138. A food composition comprising one or more of the transformed yeast strains capable of enhanced expression of any one of Aspects 2-133 or any preceding Aspect.

Aspect 139. The food composition of Aspect 137 or 138 or any preceding Aspect, comprising 1% to 40%, or 1% to 60%, and sub ranges thereof, of the one or more transformed fungal or yeast strains.

Aspect 140. The food composition of Aspect 137 or 138 or any preceding Aspect, comprising 1% to 20% (e.g., 5% to 15%, 7% to 13%, 9% to 11%, or any subrange thereof) of the one or more transformed fungal or yeast strains.

Aspect 141. The food composition of Aspect 137 or 138 or any preceding Aspect, comprising 1% to 10% (e.g., 2% to 9%, 3% to 8%, 4% to 7%, 5% to 6%, or any subrange thereof) of the one or more transformed fungal or yeast strains.

Aspect 142. The food composition of Aspect 137 or 138 or any preceding Aspect, comprising 5% to 25% (e.g., 7% to 23%, 10% to 20%, 12% to 18%, 14% to 16%, or any subrange thereof) of the one or more transformed fungal or yeast strains.

Aspect 143. The food composition of any one of Aspects 137-142 or any preceding Aspect, wherein the percentage of the one or more transformed yeast strains is based on dry weight, semi-moist weight, or wet weight of the food composition.

Aspect 144. The food composition of any one of Aspects 137-143 or any preceding Aspect, comprising two or more transformed yeast strains, optionally less than 100, optionally less than 50 transformed yeast strains.

Aspect 145. The food composition of any one of Aspects 137-143 or any preceding Aspect, comprising three or more transformed yeast strains, optionally less than 100, optionally less than 50 transformed yeast strains.

Aspect 146. The food composition of any one of Aspects 137-145 or any preceding Aspect, wherein the food composition further comprises a fat, a carbohydrate, a protein which is different from the heterologous polypeptide of interest, a fiber, a nutritional supplement, a palatability agent, or any combination thereof.

Aspect 147. The food composition of any one of Aspects 137-146 or any preceding Aspect, wherein the food composition is nutritionally balanced for a companion animal.

Aspect 148. The food composition of Aspect 147 or any preceding Aspect, wherein the companion animal is a human, a dog, a cat, a bird, a fish, a rodent, a reptile, a ferret, a hedgehog, a rabbit, a horse, a cow, a pig, a goat, a sheep, a buffalo, a water buffalo, or a chicken.

Aspect 149. A method of making a food composition comprising:
culturing at least one of the transformed fungal strains of claim 1 or the transformed yeast strains of any one of Aspects 2-136 or any preceding Aspect to produce a culture;
harvesting the heterologous polypeptide of interest from the culture to produce a harvested heterologous polypeptide of interest;
thereby making a food composition.

Aspect 150. The method of Aspect 149 or any preceding Aspect, wherein the harvesting does not comprise purifying the heterologous polypeptide of interest.

Aspect 151. The method of Aspect 149 or any preceding Aspect, wherein the harvesting the heterologous polypeptide of interest from the culture comprises obtaining whole-cells, a cell lysate, a cell supernatant, a polypeptide concentrate, or a polypeptide isolate.

Aspect 152. The method of Aspect 149 or any preceding Aspect, wherein the harvesting the heterologous polypeptide of interest from the culture comprises obtaining whole-cells.

Aspect 153. The method of any one of Aspects 149-152 or any preceding Aspect, further comprising dehydrating the harvested heterologous polypeptide of interest, thereby producing an intermediate dry food ingredient.

Aspect 154. The method of any one of Aspects 149-153 or any preceding Aspect, further comprising the subsequent steps of: concentrating, dehydrating, and rehydrating the harvested heterologous polypeptide of interest, thereby producing an intermediate wet food ingredient.

Aspect 155. Use of any one of the preceding Aspects or strains of any preceding Aspect to make a food composition.

Aspect 156. A pharmaceutical composition comprising at least one of the transformed fungal strains of Aspect 1 or the transformed yeast strains of any one of Aspects 2-136 or any preceding Aspect and a pharmaceutically acceptable carrier.

Aspect 157. A method for making a medicament comprising combining at least one of the transformed fungi strains of Aspect 1 or the transformed yeast strains of any one of Aspects 2-136 or any preceding Aspect with a pharmaceutically acceptable carrier.

Aspect 158. A food ingredient composition comprising:
a recombinant fungi, wherein the genome of the recombinant fungi comprises at least 2 copies (e.g., at least 2 copies, at least 3 copies, at least 4 copies, at least 5 copies, at least 6 copies, at least 7 copies, at least 8 copies, at least 9 copies, at least 10 copies, at least 15 copies, at least 20 copies, at least 25 copies, at least 30 copies, at least 40 copies, at least 50 copies, at least 75 copies, at least 100 copies, at least 200 copies, at least 250 copies, optionally less than 1000 copies, optionally less than 750 copies) of a tandem amplification region integrated into a locus of a haploinsufficient gene tied to fungi fitness, wherein the tandem amplification region comprises:
a coding sequence (CDS) of one or more heterologous animal proteins in operable linkage with a first promoter and a terminator, wherein the one or more heterologous animal proteins:
have less than 25% (e.g., less than 25%, less than 10%, less than 7.5%, less than 5%, less than 2.5%) amino acid sequence identity to proteins endogenous to said fungi; and
   are characterized by a molecular weight between 5 kDa and 80 kDa (e.g., between 5 kDa and 80 kDa, between 5 kDa and 60 kDa, between 5 kDa and 50 kDa, between 10 kDa and 60 kDa, and any subrange thereof);
at least 5% of the one or more heterologous animal protein by dry cell weight; and
at least 50% total protein by dry cell weight.

Aspect 159. The food ingredient composition of Aspect 158, or any preceding Aspect, wherein the one or more heterologous animal proteins bind hydrophobic molecules.

Aspect 160. The food ingredient composition of Aspect 159, or any preceding Aspect, wherein the hydrophobic molecules comprise fatty acids, lipids, or retinol.

Aspect 161. The food ingredient composition of any one of Aspects 158-160, or any preceding Aspect, wherein the one or more heterologous animal proteins are characterized by a molecular weight between 10 kDa and 45 kDa.

Aspect 162. The food ingredient composition of any one of Aspects 158-161, or any preceding Aspect, wherein the genome of the recombinant fungi comprises at least 5 copies (e.g., at least 5 copies, at least 6 copies, at least 7 copies, at least 8 copies, at least 9 copies, at least 10 copies, at least 15 copies, at least 20 copies, at least 25 copies, at least 30 copies, at least 40 copies, at least 50 copies, at least 75 copies, at least 100 copies, at least 200 copies, at least 250 copies, optionally less than 1000 copies, optionally less than 750 copies) of a tandem amplification region.

Aspect 163. The food ingredient composition of any one of Aspects 158-162, or any preceding Aspect, wherein the genome of the recombinant fungi comprises at least 10 copies (e.g., at least 10 copies, at least 15 copies, at least 20 copies, at least 25 copies, at least 30 copies, at least 40 copies, at least 50 copies, at least 75 copies, at least 100 copies, at least 200 copies, at least 250 copies, optionally less than 1000 copies, optionally less than 750 copies) of a tandem amplification region.

Aspect 164. The food ingredient composition of any one of Aspects 158-163, or any preceding Aspect, wherein the genome of the recombinant fungi comprises at least 15 copies (e.g., at least 15 copies, at least 20 copies, at least 25 copies, at least 30 copies, at least 40 copies, at least 50 copies, at least 75 copies, at least 100 copies, at least 200 copies, at least 250 copies, optionally less than 1000 copies, optionally less than 750 copies) of a tandem amplification region.

Aspect 165. The food ingredient composition of any one of Aspects 158-164, or any preceding Aspect, wherein the genome of the recombinant fungi comprises at least 30 copies (e.g., at least 30 copies, at least 40 copies, at least 50 copies, at least 75 copies, at least 100 copies, at least 200 copies, at least 250 copies, optionally less than 1000 copies, optionally less than 750 copies) of a tandem amplification region.

Aspect 166. The food ingredient composition of any one of Aspects 158-165, or any preceding Aspect, wherein the one or more heterologous animal proteins are codon-optimized for expression in Saccharomyces cerevisiae.

Aspect 167. The food ingredient composition of any one of Aspects 158-166, or any preceding Aspect, wherein at least one of the one or more heterologous animal proteins has at least 75% (e.g., at least 75%, at least 85%, at least 90%, at least 95%, at least 99%, or 100%) amino acid identity to any one of SEQ ID NOs: 1-352.

Aspect 168. The food ingredient composition of any one of Aspects 158-167, or any preceding Aspect, wherein at least one of the one or more the heterologous animal proteins has at least 75% (e.g., at least 75%, at least 85%, at least 90%, at least 95%, at least 99%, or 100%) amino acid identity to the proteins listed in Table 1, Table 11, or Table 12.

Aspect 169. The food ingredient composition of any one of Aspects 158-167, or any preceding Aspect, wherein at least one of the one or more the heterologous animal proteins has at least 75% (e.g., at least 75%, at least 85%, at least 90%, at least 95%, at least 99%, or 100%) amino acid identity to the proteins listed in Table 2.

Aspect 170. The food ingredient composition of any one of Aspects 158-167, or any preceding Aspect, wherein at least one of the one or more the heterologous animal proteins has at least 75% (e.g., at least 75%, at least 85%, at least 90%, at least 95%, at least 99%, or 100%) amino acid identity to the proteins listed in Table 3.

Aspect 171. The food ingredient composition of any one of Aspects 158-167, or any preceding Aspect, wherein at least one of the one or more the heterologous animal proteins has at least 75% (e.g., at least 75%, at least 85%, at least 90%, at least 95%, at least 99%, or 100%) amino acid identity to the proteins listed in Table 4.

Aspect 172. The food ingredient composition of any one of Aspects 158-167, or any preceding Aspect, wherein at least one of the one or more the heterologous animal proteins is an ortholog of a chicken protein of Table 4.

Aspect 173. The food ingredient composition of any one of Aspects 158-172, or any preceding Aspect, wherein the haploinsufficient gene is selected from the group consisting of RPL25, SEC23, RPL33A, RPS15, RPC10, RPS5, ACT1, NIP1, RPS13, NUS1, SMC1, RNA14, RPB7, SPC97, STH1, ARP7, TAF61, RPN11, RPL17A, RPL18A, RPS20, CCT2, CCT4, CCT6 RPB3, RPB4, RPB5, RPB8, SRB7, RPO26, CDC47, SUI2 and RVB2.

Aspect 174. The food ingredient composition of any one of Aspects 158-173, or any preceding Aspect, wherein the haploinsufficient gene is a gene encoding a transcriptional protein, a copper resistance protein, a coat protein complex, or a ribosomal subunit protein.

Aspect 175. The food ingredient composition of any one of Aspects 158-174, or any preceding Aspect, wherein the recombinant fungi contains a lower content of the haploinsufficient gene than a wild-type fungi of the same type.

Aspect 176. The food ingredient composition of Aspect 175, or any preceding Aspect, wherein lower content of the haploinsufficent gene than the wild-type fungi of the same type is attributed to:
a. the genome of the recombinant fungi comprising a second promoter in operable linkage to the CDS of the haploinsufficient gene, wherein the second promoter is weaker than the native promoter of the haploinsufficient gene;
b. a modification of the haploinsufficient gene, wherein the modification comprises an RNA destabilizing element;
c. a mutation of the haploinsufficient gene, wherein the mutation comprises a substitution or an addition of a codon of the genome of the haploinsufficient gene, wherein the codon has a lower translational efficiency as compared to the wild-type haploinsufficient gene under the same conditions;
d. the expression of a nucleic acid molecule in the recombinant fungi wherein the nucleic acid molecule reduces the level of expression of the haploinsufficient gene; and/or
e. a disruption of the haploinsufficient gene.

Aspect 177. The food ingredient composition of any one of Aspects 158-176, or any preceding Aspect, wherein the genome of the recombinant fungi comprises a second promoter in operable linkage to the CDS of the haploinsufficient gene, wherein the second promoter is weaker than the native promoter of the haploinsufficient gene.

Aspect 178. The food ingredient composition of Aspect 177, or any preceding Aspect, wherein the second promoter that is weaker than the native promoter of the haploinsufficient gene is selected from the group consisting of the ERG 1 promoter, PDA1 promoter, BTS1 promoter, GLO2 promoter and COG7 promoter.

Aspect 179. The food ingredient composition of Aspect 177 or 178, or any preceding Aspect, wherein the second promoter that is weaker than the native promoter of the haploinsufficient gene is a truncated or mutated version of the native promoter.

Aspect 180. The food ingredient composition of any one of Aspects 158-179, or any preceding Aspect, comprising at least 10% of the one or more heterologous animal protein by dry cell weight.

Aspect 181. The food ingredient composition of any one of Aspects 158-180, or any preceding Aspect, comprising at least 15% of the one or more heterologous animal protein by dry cell weight.

Aspect 182. The food ingredient composition of any one of Aspects 158-181, or any preceding Aspect, comprising at least 25% of the one or more heterologous animal protein by dry cell weight.

Aspect 183. The food ingredient composition of any one of Aspects 158-182, or any preceding Aspect, comprising at least 50% of the one or more heterologous animal protein by dry cell weight.

Aspect 184. The food ingredient composition of any one of Aspects 158-183, or any preceding Aspect, comprising at least 60% of the one or more heterologous animal protein by dry cell weight.

Aspect 185. The food ingredient composition of any one of Aspects 158-184, or any preceding Aspect, comprising at least 65% of the one or more heterologous animal protein by dry cell weight.

Aspect 186. The food ingredient composition of any one of Aspects 158-185, or any preceding Aspect, wherein the tandem amplification region comprises a coding sequence (CDS) of two or more heterologous animal proteins (e.g., two or more, three or more, four or more, five or more, six or more, seven or more, eight or more, nine or more, ten or more, fifteen or more, twenty or more, fifty or more, optionally less than one-hundred or more).

Aspect 187. The food ingredient composition of any one of Aspects 158-186, or any preceding Aspect, wherein the tandem amplification region comprises a coding sequence (CDS) of three or more heterologous animal proteins (e.g., three or more, four or more, five or more, six or more, seven or more, eight or more, nine or more, ten or more, fifteen or more, twenty or more, fifty or more, optionally less than one-hundred or more).

Aspect 188. The food ingredient composition of any one of Aspects 158-187, or any preceding Aspect, wherein the tandem amplification region comprises a coding sequence (CDS) of four or more heterologous animal proteins (e.g., four or more, five or more, six or more, seven or more, eight or more, nine or more, ten or more, fifteen or more, twenty or more, fifty or more, optionally less than one-hundred or more).

Aspect 189. The food ingredient composition of any one of Aspects 158-188, or any preceding Aspect, wherein the tandem amplification region comprises a coding sequence (CDS) of five or more heterologous animal proteins (e.g., five or more, six or more, seven or more, eight or more, nine or more, ten or more, fifteen or more, twenty or more, fifty or more, optionally less than one-hundred or more).

Aspect 190. The food ingredient composition of any one of Aspects 158-189, or any preceding Aspect, wherein the tandem amplification region comprises a coding sequence (CDS) of six or more heterologous animal proteins (e.g., six or more, seven or more, eight or more, nine or more, ten or more, fifteen or more, twenty or more, fifty or more, optionally less than one-hundred or more).

Aspect 191. The food ingredient composition of any one of Aspects 158-190, or any preceding Aspect, wherein the genome of the recombinant fungi comprises at least 5 copies of a tandem amplification region (e.g., at least 2 copies, at least 3 copies, at least 4 copies, at least 5 copies, at least 6 copies, at least 7 copies, at least 8 copies, at least 9 copies, at least 10 copies, at least 15 copies, at least 20 copies, at least 25 copies, at least 30 copies, at least 40 copies, at least 50 copies, at least 75 copies, at least 100 copies, at least 200 copies, at least 250 copies, optionally less than 1000 copies, optionally less than 750 copies).

Aspect 192. The food ingredient composition of any one of Aspects 158-191, or any preceding Aspect, wherein the genome of the recombinant fungi comprises at least 10 copies of a tandem amplification region (e.g., at least 10 copies, at least 15 copies, at least 20 copies, at least 25 copies, at least 30 copies, at least 40 copies, at least 50 copies, at least 75 copies, at least 100 copies, at least 200 copies, at least 250 copies, optionally less than 1000 copies, optionally less than 750 copies).

Aspect 193. The food ingredient composition of any one of Aspects 158-192, or any preceding Aspect, wherein the genome of the recombinant fungi comprises at least 20 copies of a tandem amplification region (e.g., at least 20 copies, at least 25 copies, at least 30 copies, at least 40 copies, at least 50 copies, at least 75 copies, at least 100 copies, at least 200 copies, at least 250 copies, optionally less than 1000 copies, optionally less than 750 copies).

Aspect 194. The food ingredient composition of any one of Aspects 158-193, or any preceding Aspect, wherein the genome of the recombinant fungi comprises at least 30 copies of a tandem amplification region (e.g., at least 30 copies, at least 40 copies, at least 50 copies, at least 75 copies, at least 100 copies, at least 200 copies, at least 250 copies, optionally less than 1000 copies, optionally less than 750 copies).

Aspect 195. The food ingredient composition of any one of Aspects 158-194, or any preceding Aspect, wherein the genome of the recombinant fungi comprises at least 40 copies of a tandem amplification region (e.g., at least 40 copies, at least 50 copies, at least 75 copies, at least 100 copies, at least 200 copies, at least 250 copies, optionally less than 1000 copies, optionally less than 750 copies).

Aspect 196. The food ingredient composition of any one of Aspects 158-195, or any preceding Aspect, wherein the genome of the recombinant fungi comprises at least one other tandem amplification region integrated into a locus of a second haploinsufficient gene tied to fungi fitness.

Aspect 197. The food ingredient composition of any one of Aspects 158-196, or any preceding Aspect, wherein the composition has less than 10% (e.g., less than 10%, less than 9%, less than 8%, less than 7.5%, less than 6%, less than 5%) crude fat by dry cell weight.

Aspect 198. The food ingredient composition of any one of Aspects 158-197, or any preceding Aspect, wherein the composition has less than 7.5% (e.g., less than 7.5%, less than 6% less than 5%) crude fat by dry cell weight.

Aspect 199. The food ingredient composition of any one of Aspects 158-198, or any preceding Aspect, wherein the composition has less than 5% crude fat by dry cell weight.

Aspect 200. The food ingredient composition of any one of Aspects 158-199, or any preceding Aspect, wherein the composition has less than 10% (e.g., less than 10%, less than 9%, less than 8%, less than 7.5%, less than 6% less than 5%) ash by dry cell weight.

Aspect 201. The food ingredient composition of any one of Aspects 158-185, or any preceding Aspect, wherein the composition has less than 7.5% (e.g., less than 7.5%, less than 6%, less than 5%) ash by dry cell weight.

Aspect 202. The food ingredient composition of any one of Aspects 158-201, or any preceding Aspect, wherein the composition has less than 5% ash by dry cell weight.

Aspect 203. The food ingredient composition of any one of Aspects 158-202, or any preceding Aspect, wherein the composition comprises B vitamins.

Aspect 204. The food ingredient composition of any one of Aspects 158-203, or any preceding Aspect, wherein the composition has less than 1% (e.g., less than 1%, less than 0.9%, less than 0.8%, less than 0.75%, less than 0.6% less than 0.5%, less than 0.4%, less than 0.3%, less than 0.25%) sodium by dry cell weight.

Aspect 205. The food ingredient composition of any one of Aspects 158-204, or any preceding Aspect, wherein the composition has less than 0.75% (e.g., less than 0.75%, less than 0.6% less than 0.5%, less than 0.4%, less than 0.3%, less than 0.25%) sodium by dry cell weight.

Aspect 206. The food ingredient composition of any one of Aspects 158-205, or any preceding Aspect, wherein the composition has less than 0.3% (e.g., less than 0.3%, less than 0.25%) sodium by dry cell weight.

Aspect 207. The food ingredient composition of any one of Aspects 158-206, or any preceding Aspect, wherein the one or more heterologous animal proteins have an amino acid sequence identity comprising between 2% and 5% (e.g., between 2.1% and 4.6%, between 2.5% and 3.8%, preferably about 2.9%) arginine residues relative to the total number of amino acid residues in the sequence.

Aspect 208. The food ingredient composition of any one of Aspects 158-207, or any preceding Aspect, wherein the one or more heterologous animal proteins have an amino acid sequence identity comprising between 0.75% and 2% (e.g., between 0.8% and 1.9%, between 1% and 1.6%, preferably about 1.2%) histidine residues relative to the total number of amino acid residues in the sequence.

Aspect 209. The food ingredient composition of any one of Aspects 158-208, or any preceding Aspect, wherein the one or more heterologous animal proteins have an amino acid sequence identity comprising between 1.5% and 4.5% (e.g., between 1.7% and 4.3%, between 2.2% and 3.5%, preferably about 2.7%) isoleucine residues relative to the total number of amino acid residues in the sequence.

Aspect 210. The food ingredient composition of any one of Aspects 158-209, or any preceding Aspect, wherein the one or more heterologous animal proteins have an amino acid sequence identity comprising between 2.5% and 8% (e.g., between 2.9% and 6.7%, between 3.5% and 5.5%, preferably about 4.2%) leucine residues relative to the total number of amino acid residues in the sequence.

Aspect 211. The food ingredient composition of any one of Aspects 158-210, or any preceding Aspect, wherein the one or more heterologous animal proteins have an amino acid sequence identity comprising between 2% and 6% (e.g., between 2.4% and 5.6%, between 3% and 4.6%, preferably about 3.5%) lysine residues relative to the total number of amino acid residues in the sequence.

Aspect 212. The food ingredient composition of any one of Aspects 158-211, or any preceding Aspect, wherein the one or more heterologous animal proteins have an amino acid sequence identity comprising between 0.5% and 3% (e.g., between 0.7% and 2.4%, between 1.3% and 2%, preferably about 1.5%) methionine residues relative to the total number of amino acid residues in the sequence.

Aspect 213. The food ingredient composition of any one of Aspects 158-212, or any preceding Aspect, wherein the one or more heterologous animal proteins have an amino acid sequence identity comprising between 1% and 5% (e.g., between 1.4% and 4.2%, between 2.2% and 3.4%, preferably about 2.6%) phenylalanine residues relative to the total number of amino acid residues in the sequence.

Aspect 214. The food ingredient composition of any one of Aspects 158-213, or any preceding Aspect, wherein the one or more heterologous animal proteins have an amino acid sequence identity comprising between 1% and 4.5% (e.g., between 1.6% and 3.8%, between 2% and 3.1%, preferably about 2.35%) threonine residues relative to the total number of amino acid residues in the sequence.

Aspect 215. The food ingredient composition of any one of Aspects 158-214, or any preceding Aspect, wherein the one or more heterologous animal proteins have an amino acid sequence identity comprising between 0.25% and 1.5% (e.g., between 0.4% and 1%, between 0.5% and 0.8%, preferably about 0.6%) tryptophan residues relative to the total number of amino acid residues in the sequence.

Aspect 216. The food ingredient composition of any one of Aspects 158-215, or any preceding Aspect, wherein the one or more heterologous animal proteins have an amino acid sequence identity comprising between 1.5% and 6% (e.g., between 2% and 4.8%, between 2.6% and 3.9%, preferably about 3%) valine residues relative to the total number of amino acid residues in the sequence.

Aspect 217. The food ingredient composition of any one of Aspects 158-216, or any preceding Aspect, wherein the one or more heterologous animal proteins have not been purified away from the recombinant fungi cells.

Aspect 218. The food ingredient composition of any one of Aspects 158-217, or any preceding Aspect, wherein the composition comprises whole cells, a cell lysate, or partially lysed cells comprising the genome of the recombinant fungi cells.

Aspect 219. The food ingredient composition of any one of Aspects 158-218, or any preceding Aspect, wherein the fungi is yeast.

Aspect 220. The food ingredient composition of any one of Aspects 158-219, or any preceding Aspect, wherein the one or more heterologous animal proteins are annexins, fatty acid binding proteins or retinoid binding proteins.

Aspect 221. The food ingredient composition of any one of Aspects 158-220, or any preceding Aspect, wherein the one or more heterologous animal proteins comprise at least a portion of a protein from a vertebrate animal.

Aspect 222. The food ingredient composition of Aspect 221, or any preceding Aspect, wherein the vertebrate animal is a pig, a turkey, a chicken, a pheasant, a quail, a horse, a cow, a fish, a sheep, a deer, a red deer, a duck, a rabbit, an elk, a moose, a kangaroo, an alligator, a lamb, a wild boar, a goat, a bison, a buffalo or a water buffalo.

Aspect 223. The food ingredient composition of Aspect 221 or 222, or any preceding aspect, wherein the protein from the vertebrate animal is a muscle protein.

Aspect 224. A food ingredient composition comprising recombinant fungi, wherein
a. the recombinant fungi is transformed with a vector that creates at least 2 copies (e.g., at least 2 copies, at least 3 copies, at least 4 copies, at least 5 copies, at least 6 copies, at least 7 copies, at least 8 copies, at least 9 copies, at least 10 copies, at least 15 copies, at least 20 copies, at least 25 copies, at least 30 copies, at least 40 copies, at least 50 copies, at least 75 copies, at least 100 copies, at least 200 copies, at least 250 copies, optionally less than 1000 copies, optionally less than 750 copies) of a tandem amplification region integrated into the genome of the fungi at a locus of a haploinsufficient gene tied to fungi fitness, wherein the tandem amplification region:
   i. encodes one or more heterologous animal proteins, wherein the one or more heterologous animal proteins comprise:
      1. less than less than 25% (e.g., less than 25%, less than 10%, less than 7.5%, less than 5%, less than 2.5%) amino acid identity with proteins endogenous to said fungi; and
      2. a molecular weight of between 5 kDa and 80 kDa;
b. the composition comprises at least 5% of the one or more heterologous animal protein by dry cell weight; and
c. the composition comprises at least 50% total protein by dry cell weight.

Aspect 225. The food ingredient composition of Aspect 224, or any preceding aspect, wherein the vector comprises:
a. one or more genes encoding heterologous animal proteins, wherein each gene is in operable linkage with the first promoter and the first terminator:
b. a second promoter homologous to at least a portion of the native promoter region of the haploinsufficient gene tied to fungi fitness;
c. a second terminator homologous to at least a portion of the native terminator of the haploinsufficient gene; and
d. a synthetic open reading frame (ORF) that is homologous to at least a portion of a native ORF of the haploinsufficient gene;
wherein elements (a), (c), (d), and a native terminator region of the haploinsufficient gene form the tandem amplification region that is integrated into the genome of the fungi.

Aspect 226. The food ingredient composition of Aspect 224 or 225, or any preceding Aspect, wherein the vector further comprises a selectable marker.

Aspect 227. The food ingredient composition of Aspect 226, or any preceding Aspect, wherein the selectable marker is removable after integration of the vector into the genome of the fungi.

Aspect 228. The food ingredient composition of Aspect 226, or any preceding Aspect, wherein the selectable marker is flanked by loxP sites and is removed by expression of a Cre protein after integration of the vector(s) into the genome of the fungi.

Aspect 229. The food ingredient composition of Aspect 224 or 225, or any preceding Aspect, wherein the vector does not comprise a selectable marker, and wherein selective pressure is provided by a plasmid encoding a selectable marker that has been co-transformed with the vector such that the tandem amplification region does not contain a selectable marker.

Aspect 230. The food ingredient composition of any preceding Aspect wherein elements (a) through (d) form a DNA fragment that is integrated into the genome of the fungi at the locus of the haploinsufficient gene through CRISPR nuclease-guide RNA (gRNA) mediated recombination.

Aspect 231. A food ingredient composition comprising recombinant fungi, wherein:
a. the recombinant fungi is transformed with a vector that creates at least 2 copies of a tandem amplification region integrated into the genome of the fungi at a locus of a haploinsufficient gene tied to fungi fitness, wherein the tandem amplification region:
   i. encodes one or more heterologous animal proteins that have at least 75% amino acid sequence identity to the proteins of SEQ ID NOs. 1-352;
   ii. functions to reduce the expression of the haploinsufficient gene;
b. the food ingredient composition comprises:
   i. at least 5% heterologous animal protein by dry cell weight;
   ii. at least 50% total protein by dry cell weight.

Aspect 232. The food ingredient composition of Aspect 231, or any preceding Aspect, wherein the vector comprises:
a. one or more genes encoding the one or more heterologous animal proteins, each gene in operable linkage with a first promoter and a first terminator;
b. a second promoter homologous to at least a portion of the native promoter region of the haploinsufficient gene tied to fungi fitness;
c. a second terminator homologous to at least a portion of the native terminator of the haploinsufficient gene, wherein the second promoter is weaker than the native promoter of the haploinsufficient gene; and
d. a synthetic open reading frame (ORF) homologous to at least a portion of a native ORF of the haploinsufficient gene;
   wherein elements (a), (c), (d), and the native terminator region of the haploinsufficient gene form the tandem amplification region that is integrated into the genome of the fungi at the locus of the haploinsufficient gene.

Aspect 233. The food ingredient composition of Aspect 232, or any preceding Aspect, wherein the vector does not contain a origin of replication or an autonomously replicating sequence (ARS).

Aspect 234. The food ingredient composition of any one of Aspect 231, or any preceding Aspect, wherein the recombinant fungi contains a lower content of the haploinsufficient gene than a wild-type fungi of the same type.

Aspect 235. The food ingredient composition of Aspect 231, or any preceding Aspect, wherein lower content of the haploinsufficient gene than the wild-type fungi of the same type is attributed to:
a. the genome of the recombinant fungi comprising a second promoter in operable linkage to the CDS of the haploinsufficient gene, wherein the second promoter is weaker than the native promoter of the haploinsufficient gene;
b. a modification of the haploinsufficient gene, wherein the modification comprises an RNA destabilizing element;
c. a mutation of the haploinsufficient gene, wherein the mutation comprises a substitution or an addition of a codon of the genome of the haploinsufficient gene, wherein the codon has a lower translational efficiency as compared to the wild-type haploinsufficient gene under the same conditions;
d. the expression of a nucleic acid molecule in the recombinant fungi wherein the nucleic acid molecule reduces the level of expression of the haploinsufficient gene; and/or
e. a disruption of the haploinsufficient gene.

A recombinant fungi, wherein the genome of the recombinant fungi comprises at least 2 copies (e.g., at least 2 copies, at least 3 copies, at least 4 copies, at least 5 copies, at least 6 copies, at least 7 copies, at least 8 copies, at least 9 copies, at least 10 copies, at least 15 copies, at least 20 copies, at least 25 copies, at least 30 copies, at least 40 copies, at least 50 copies, at least 75 copies, at least 100 copies, at least 200 copies, at least 250 copies, optionally less than 1000 copies, optionally less than 750 copies) of a tandem amplification region integrated into a locus of a haploinsufficient gene tied to fungi fitness, wherein the tandem amplification region comprises:
a coding sequence (CDS) of one or more heterologous animal proteins in operable linkage with a promoter and a terminator, wherein the one or more heterologous animal proteins:
   have less than 25% amino acid sequence identity with proteins endogenous to said fungi; and
   is characterized by a molecular weight between 5 kDa and 80 kDa.

### EXAMPLES

The invention can be further understood by the following non-limiting examples. The examples are provided to illustrate some of the concepts described within this disclosure. While each example is considered to provide specific individual embodiments of composition and methods of preparation and use, none of the examples should be considered to limit the more general embodiments described herein.

In the following examples, efforts have been made to ensure accuracy with respect to numbers used (e.g., amounts, temperature, etc.) but some experimental error and deviation should be accounted for. The practice of the present invention will employ, unless otherwise indicated, conventional methods of protein chemistry, biochemistry, recombinant DNA techniques and pharmacology used in the art. Also referred to below are the following references, each of which is hereby incorporated by reference in its entirety for all purposes: M. R. Green and J. Sambrook, Molecular Cloning: A Laboratory Manual, 4th Edition, Cold Spring Harbor, NY: Cold Spring Harbor Laboratory Press, 2012, pp. 1009-1011; G. C. U. F. T. Tool, GenScript, [Online]. Available: https://www.genscript.com/tools/codon-frequencytable; S. Wu and L. J. Geoffrey, “High efficiency transformation by electroporation of Pichia pastoris pretreated with lithium acetate and dithiothreitol,” Drug Discovery and genomic technologies, vol. 36, no. 1, pp. 152-154, 2004; S. Kawai et al., “Transformation of Saccharomyces cerevisiae and other fungi,” Bioengineered Bugs, vol. 1, no. 6, pp. 395-403, 2010; P. Manivasakam and R. H. Schiestl, “High efficiency transformation of Saccharomyces cerevisiae by electroporation” Nucleic Acids Research, vol. 21, no. 18, pp. 4414-4415, 1993.

In the following examples, the term “S #” refers to a transformed yeast strain expressing a heterologous animal protein. For example, “S1” refers to strain 1. The contents of each strain are provided in Table 5 below. Table 5 provides the animal species, protein name, promoter and terminator combination used. The percent of target animal protein, Protein Of Interest (POI) has been calculated using the Total Protein Approach (TPA method). Calculations are performed as described in Methods in Enzymology Vol 585 (Proteomics in Biology, Part A) Chapter 4—Label Free and Standard Free Absolute Quantitative Proteomics using the “Total Protein” and “Proteomic Ruler” Approaches, which is hereby incorporated by reference in its entirety for all purposes, including for the description of the total protein and proteomic ruler approaches. The “Total Protein Approach” (TPA), allows absolute protein quantitation.

In the TPA method, calculation of protein abundance is based on spectral intensities acquired in the large-scale proteomic analyses (Wisniewski et al., 2017, Methods in Enzymology, Ch. 4, pgs. 49-60, which is hereby incorporated by reference in its entirety, and specifically for the large-scale proteomic analyses). The percent of protein in the samples is calculated using the following equation (E1):

$$\% Total protein ( i ) = MS - signal ( i ) / Total MS - signal \times 100 \% \qquad ( E1 )$$

where (i) refers to any given protein.

Growth rates are tabulated using the Cell Growth Quantifier and DOTS software from Scientific Bioprocessing Inc. The growth rate μ (h⁻¹), is calculated using the following equation (E2):

$$μ ( {h}^{- 1} ) = ( ln ( {D}_{f} / {D}_{i} ) ) / ( {T}_{f} - {T}_{i} ) \qquad ( E2 )$$

where D_{f} and Dᵢ stand for the final and initial culture densities, on a monotonically increasing interval spanning T_{f} (final time), and Tᵢ (initial time).

The growth rates included in the calculation are strains grown in a defined glucose medium and pertain to growth on glucose prior to the diauxic shift. Where available, the growth rate from multiple flasks were averaged to provide a more accurate representation; the number of replicates included is designated as “N=”. % POI on a CDW (Cell Dry Weight) Basis is calculated by multiplying POI (%) determined by mass spectrometry, by the fraction of total protein in the final product mass, as determined by Kjeldahl protein quantification.

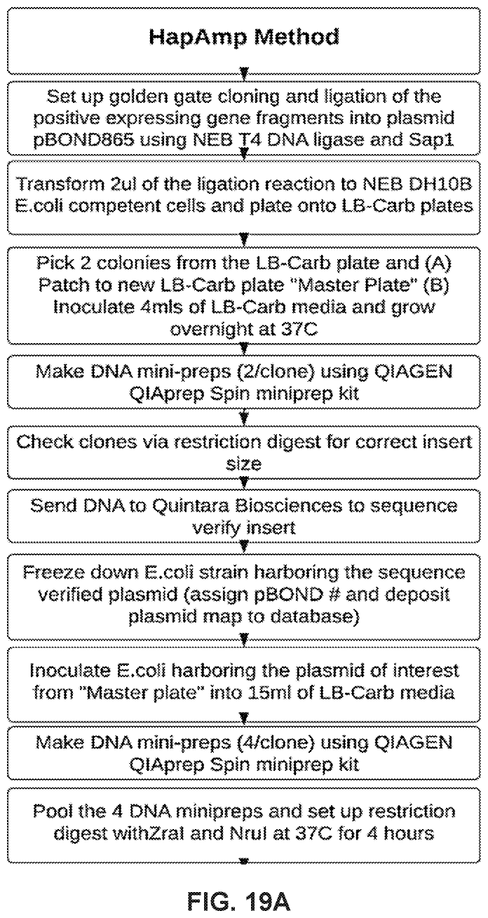

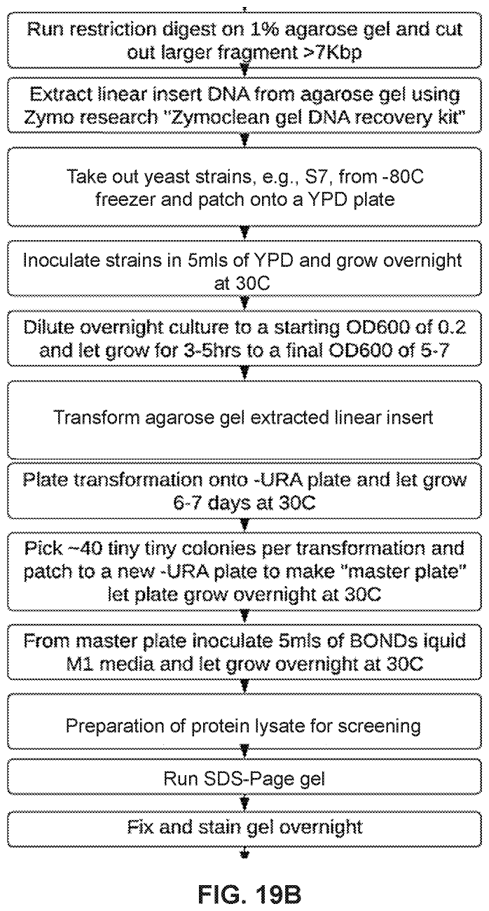

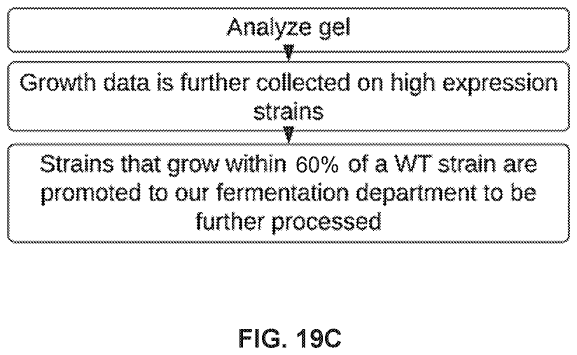

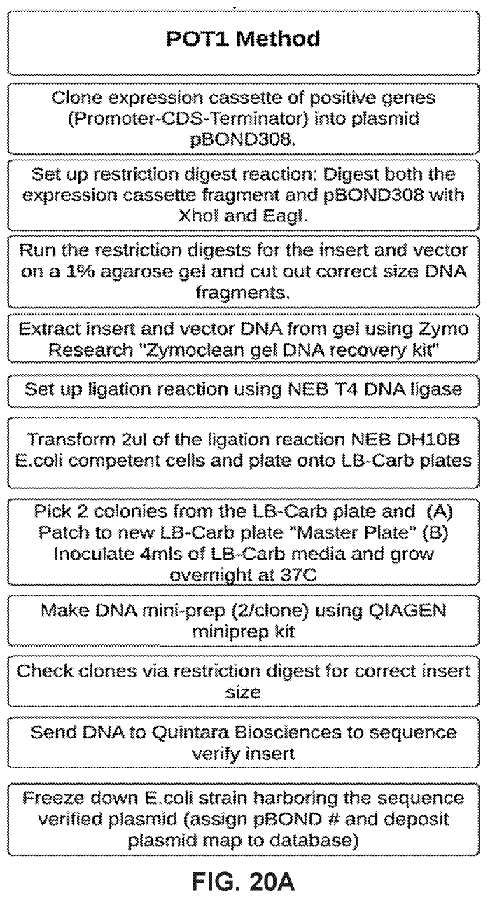

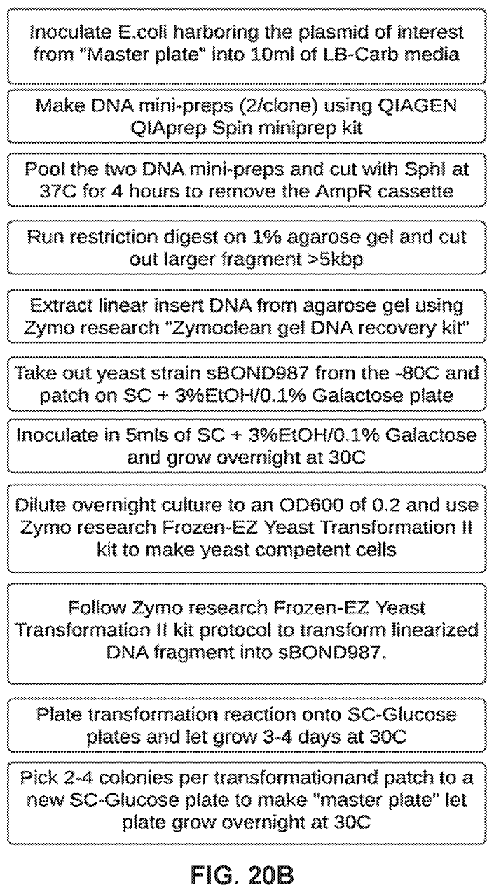

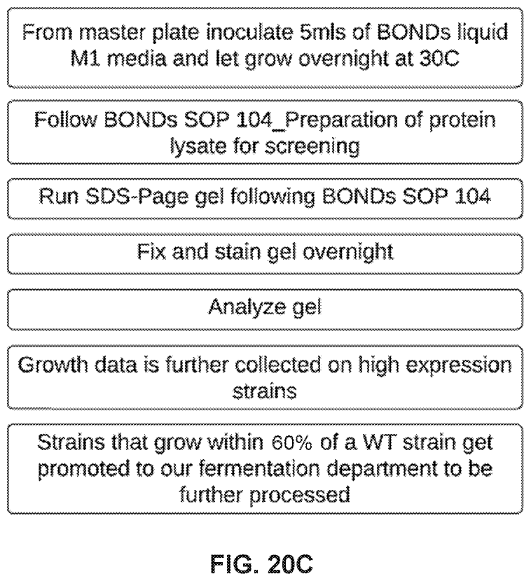

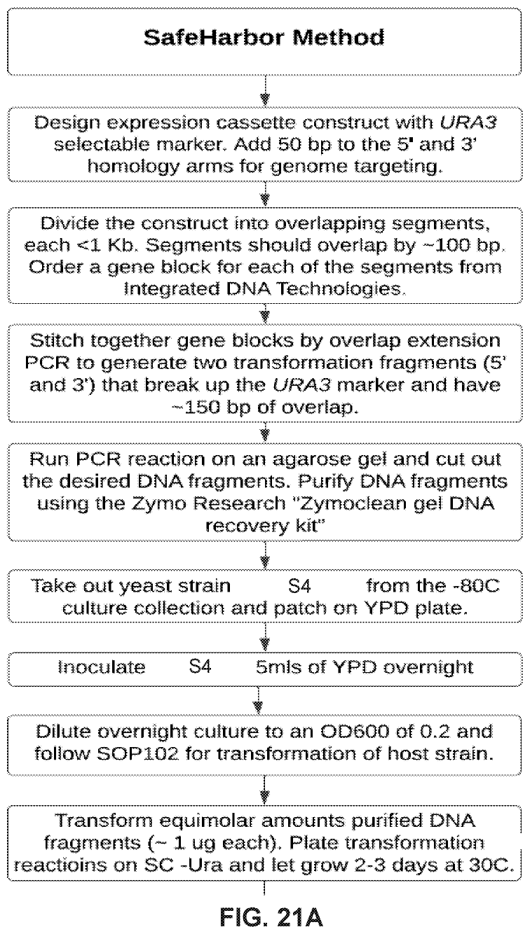

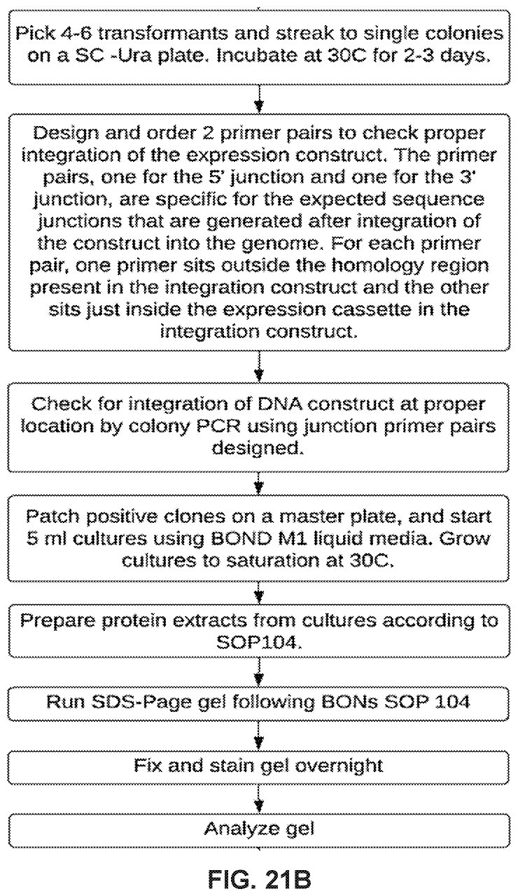

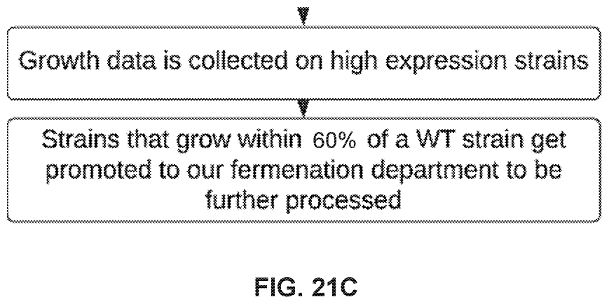

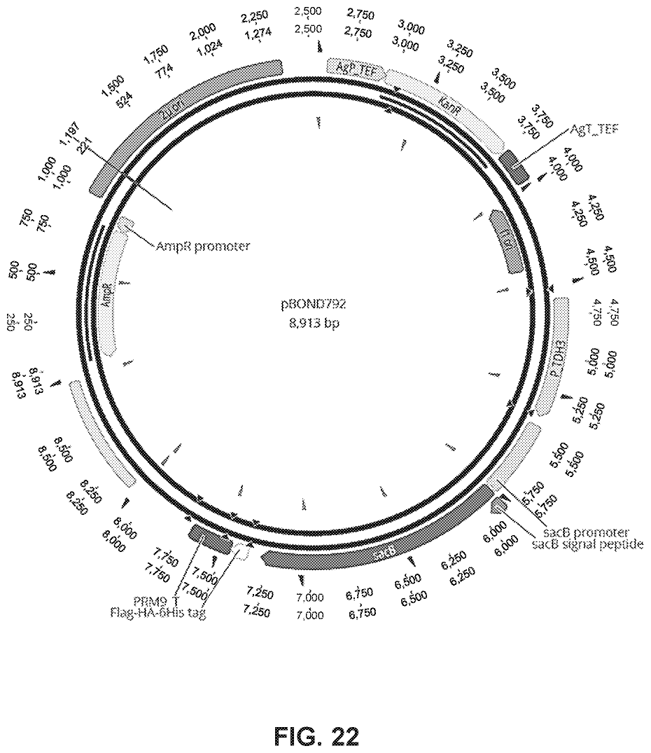

In Table 5, where an animal species, promoter, and terminator are not listed, the strain is an “unengineered” strain, meaning the strain has not be transformed with a gene expressing a heterologous protein. S1, S3, and S4 are examples of such unengineered strains. Additionally, in Table 5, where multiple proteins are listed for a single strain, said strain comprises a “protein stack” where the strain expresses two or more proteins at one, two, or three different HapAmp sites to arrive at the total protein percentage indicated. For example, S12 was transformed with a gene expressing both annexin A1 and annexin A8 which resulted in a total protein content of 51.1%. Each of the strains in Table 5 were generated using the modified HapAmp yeast transformation methods as described herein and further as described in the flowchart of FIGS. 19A-19C, and further in the following examples, specifically Examples 5-7.

TABLE 5
Example strains containing genes expressing animal proteins.

| | | | | | MS data | | | CGQ data | | Total |
|---|---|---|---|---|---|---|---|---|---|---|
| | | | | | | | POI (% | Growth | | Protein |
| Strain | Animal species | Protein | Promoter | Terminator | POI (%) | N = | per CDW) | rate(h⁻¹) | N = | (%) |
| S1 | — | None | — | — | — | — | — | 0.178 | 3 | — |
| S2 | Lamb | Annexin A3 | TEF1 | IDP1 | 8.1 | 1 | 3.8 | — | — | 47.4 |
| S3 | — | None | — | — | — | — | — | 0.289 | 4 | 42.3 |
| S4 | — | None | — | — | — | — | — | 0.254 | 1 | — |
| S7 | Lamb | Annexin A3 | TDH3 | CYC1 | 24.1 ± 4.4 | 8 | 13.9 | 0.249 | 2 | 57.5 |
| S8 | Chicken | Annexin A5 | TDH3 | CYC1 | 14.2 ± 1.2 | 6 | 6.5 | 0.225 | 3 | 45.9 |
| S9 | Chicken | Annexin A4 | TDH3 | CYC1 | 17.2 ± 3.1 | 8 | 9.0 | 0.176 | 4 | 52.5 |
| S10 | Red deer | Annexin A4 | TDH3 | PRM9 | 6.4 ± 0.7 | 2 | 3.0 | 0.155 | 1 | 46.5 |
| S11 | Red deer | Annexin A8 | TDH3 | PRM9 | 13.5 ± 2.6 | 2 | 6.3 | 0.154 | 1 | 46.4 |
| S12 | Red deer | Annexin A1 | TDH3 | PRM9 | 13.35 | 1 | 6.8 | 0.199 | 2 | 51.1 |
| | | Annexin A8 | PGK1 | TEF1 | | | | | | |
| S13 | Red deer | FABP4 | TDH3 | PRM9 | 14.9 | 1 | 7.3 | 0.223 | 2 | 49.3 |
| | | Annexin A1 | PGK1 | TEF1 | | | | | | |
| S14 | Red deer | FABP4 | TDH3 | PRM9 | 14.8 ± 0.2 | 2 | 7.5 | 0.216 | 2 | 50.6 |
| | | Annexin A4 | PGK1 | TEF1 | | | | | | |
| S15 | Red deer | Annexin A1 | TDH3 | PRM9 | 15.2 | 1 | 7.6 | 0.223 | 2 | 50.1 |
| | | Annexin A4 | PGK1 | TEF1 | | | | | | |
| S16 | Red deer | FABP4 | TDH3 | PRM9 | 15.7 | 2 | 7.9 | 0.188 | 2 | 50.6 |
| | | Annexin A1 | TDH3 | PRM9 | | | | | | |
| | | Annexin A8 | PGK1 | TEF1 | | | | | | |
| S17 | Red deer | FABP4 | TDH3 | PRM9 | 24.6 | 2 | 12.8 | 0.117 | 2 | 52 |
| | | Annexin A1 | PGK1 | TEF1 | | | | | | |
| | | Annexin A4 | TDH3 | PRM9 | | | | | | |
| S18 | Red deer | Annexin A1 | TDH3 | PRM9 | 27.2 | 8 | 14.2 | 0.163 | 2 | 52.1 |
| | | Annexin A4 | TDH3/PGK1 | PRM9/TEF1 | | | | | | |
| S19 | Red deer | FABP4 | TDH3 | CYC1 | 18.15 | 2 | 8.5 | 0.153 | 2 | 47.1 |
| | | Annexin A1 | TDH3/PGK1 | CYC1/TEF1 | | | | | | |
| | | Annexin A4 | PGK1 | TEF1 | | | | | | |
| S20 | Red deer | FABP4 | TDH3 | PRM9 | 17 | 4 | 10.5 | 0.207 | 1 | 61.7 |
| | | Annexin A1 | PGK1 | TEF1 | | | | | | |
| | | Annexin A4 | PGK1 | TEF1 | | | | | | |
| S21 | Red deer | FABP4 | TDH3 | PRM9 | 21.5 | 2 | 12.3 | 0.22 | 1 | 57 |
| | | Annexin A1 | TDH3 | PRM9 | | | | | | |
| | | Annexin A4 | PGK1 | TEF1 | | | | | | |
| | | Annexin A8 | PGK1 | TEF1 | | | | | | |
| S22 | Red deer | FABP4 | TDH3 | PRM9 | 24.6 | 2 | 14.5 | 0.174 | 1 | 59 |
| | | Annexin A1 | TDH3 | PRM9 | | | | | | |
| | | Annexin A4 | PGK1 | TEF1 | | | | | | |
| S23 | Red deer | FABP4 | TDH3 | PRM9 | 17.2 | 2 | 10.7 | 0.149 | 2 | 62.2 |
| | | Annexin A1 | PGK1 | TEF1 | | | | | | |
| | | Annexin A4 | PGK1 | TEF1 | | | | | | |
| S24 | Red deer | FABP4 | TDH3 | PRM9 | 17.5 | 2 | 10.6 | 0.167 | 2 | 60.8 |
| | | Annexin A1 | PGK1 | TEF1 | | | | | | |
| | | Annexin A4 | PGK1 | TEF1 | | | | | | |
| S25 | Red deer | FABP4 | TDH3 | PRM9 | 24.9 | 2 | 15.3 | 0.181 | 2 | 61.6 |
| | | Annexin A1 | TDH3 | PRM9 | | | | | | |
| | | Annexin A4 | PGK1 | TEF1 | | | | | | |
| S26 | Red deer | FABP4 | TDH3 | PRM9 | 19.8 | 2 | 11.4 | 0.153 | 2 | 57.7 |
| | | Annexin A1 | TDH3 | PRM9 | | | | | | |
| | | Annexin A4 | PGK1 | TEF1 | | | | | | |
| S27 | Chicken/Water | Annexin A4 | TDH3 | CYC1 | — | — | — | | | 56.9 |
| | Buffalo | FABP5 | TDH3 | PRM9 | | | | | | |
| S28 | Chicken/Water | Annexin A4 | TDH3 | CYC1 | — | — | — | | | 58.5 |
| | Buffalo | FABP5 | TDH3 | PRM9 | | | | | | |
| S29 | Chicken/Water | Annexin A4 | TDH3 | CYC1 | — | — | — | | | 59.2 |
| | Buffalo | RBP2 | TDH3 | PRM9 | | | | | | |
| S30 | Chicken/Water | Annexin A4 | TDH3 | CYC1 | — | — | — | 0.197 | 2 | 57.9 |
| | Buffalo | RBP2 | TDH3 | PRM9 | | | | | | |
| S27 | Chicken | Annexin A4 | TDH3 | PRM9 | 17.9 | 2 | 9.8 | 0.179 | 1 | 54.6 |
| S28 | Chicken | Annexin A4 | TDH3 | PRM9 | 23.71 | 2 | 13.1 | 0.185 | 1 | 55.3 |
| S29 | Chicken | Annexin A4 | TDH3 | PRM9 | 18.13 | 2 | 10.0 | — | — | 55 |
| S30 | Chicken | Annexin A4 | TDH3 | PRM9 | 17.5 | 2 | 9.3 | — | — | 53.2 |
| S31 | Red deer | FABP4 | TDH3 | PRM9 | 28.36 | 2 | 17.2 | 0.217 | 1 | 60.6 |
| | | Annexin A1 | TDH3 | PRM9 | | | | | | |
| | | Annexin A4 | PGK1 | TEF1 | | | | | | |
| | | Annexin A8 | TDH3 | PRM9 | | | | | | |
| S32 | Red deer | FABP4 | TDH3 | PRM9 | 26.7 | 2 | 17.0 | 0.105 | 1 | 63.6 |
| | | Annexin A1 | TDH3 | PRM9 | | | | | | |
| | | Annexin A4 | PGK1 | TEF1 | | | | | | |
| | | Annexin A8 | TDH3 | PRM9 | | | | | | |
| S33 | Red deer | Annexin A8 | TDH3 | PRM9 | 27.58 | 2 | 16.5 | 0.153 | 1 | 59.9 |
| | | FABP4 | TDH3 | PRM9 | | | | | | |
| | | Annexin A1 | PGK1 | TEF1 | | | | | | |
| | | Annexin A4 | TDH3 | PRM9 | | | | | | |
| S34 | Red deer | Annexin A8 | TDH3 | PRM9 | 21.84 | 2 | 13.0 | 0.188 | 1 | 59.6 |
| | | FABP4 | TDH3 | PRM9 | | | | | | |
| | | Annexin A1 | PGK1 | TEF1 | | | | | | |
| | | Annexin A4 | PGK1 | TEF1 | | | | | | |

### Example 1

This example provides the use of the POT1 system to express lamb AnxA3 protein in S. cerevisiae.

The coding sequence for lamb Annexin A3 (ANXA3) was synthesized by Twist Bioscience. Cloning of the gene into an expression vector and analysis of protein expression were done using well established molecular cloning methods (Ausubel et al. 2003).

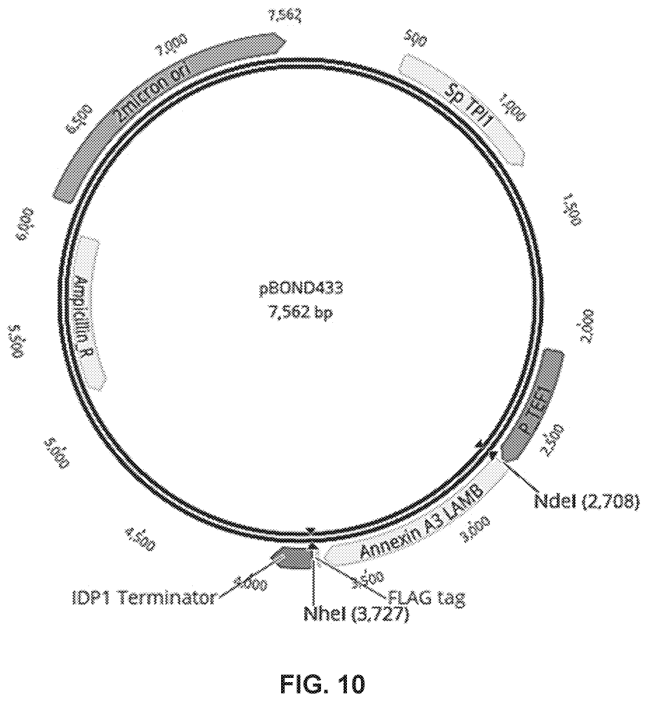

The gene was amplified by polymerase chain reaction (PCR) with primers oBOND268 and oBOND426 to add specific restriction enzyme sites on both ends of the gene. The resulting PCR fragment was digested with restriction enzymes NdeI (New England Biolabs (NEB), cat #R0111S) and NheI (NEB, cat #R3131S) and then ligated with T4 DNA ligase (NEB, cat #M0202S) into the plasmid backbone from pBOND433 (FIG. 10), which was linearized with the same restriction enzymes followed by dephosphorylation by Quick CIP phosphatase (NEB, cat #M0525S).

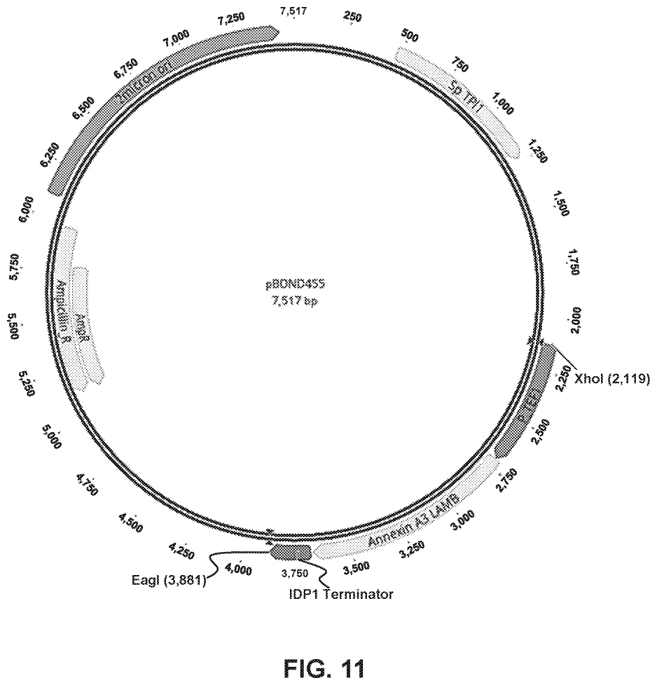

This generated vector pBOND455 (FIG. 11), with the ANXA3 expression cassette (Sc TEF1 promoter—ANXA3 coding sequence—Sc IDP1 terminator). The expression cassette is flanked by restriction sites for the XhoI (NEB, cat #R0146S) and EagI (NEB, cat #R3505S) enzymes.

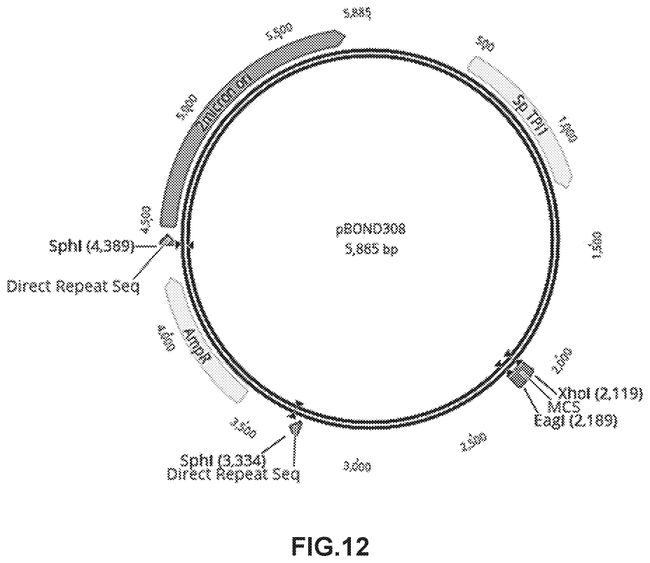

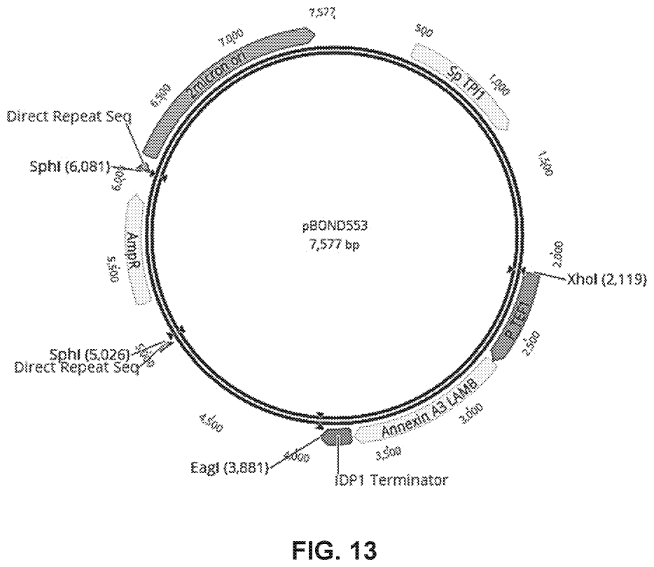

To create the POT1 expression vector, plasmid pBOND455 was digested with XhoI and EagI. The restriction digest DNA fragments were separated by gel electrophoresis, and the DNA fragment containing the ANXA3 expression cassette was purified using the Zymoclean™ Gel DNA Recovery Kit (Zymo Research, Cat #D4001). The purified expression cassette was inserted into the plasmid backbone from pBOND308 (FIG. 12), which was also linearized with XhoI and EagI, followed by dephosphorylation with Quick CIP phosphatase, resulting in plasmid pBOND553 (FIG. 13).

Plasmid pBOND553 contains a 2-micron origin of replication for high copy replication in S. cerevisiae (Sc), the ANXA3 expression cassette, and the S. pombe tpi1+ gene, which is used as a selectable marker as it has been shown to functionally complement a tpi1 deletion in S. cerevisiae. It also contains an Ampicillin resistance cassette, which is flanked on both sides by SphI (NEB, cat #R3182S) restriction sites and two 50 bp long direct repeat sequences.

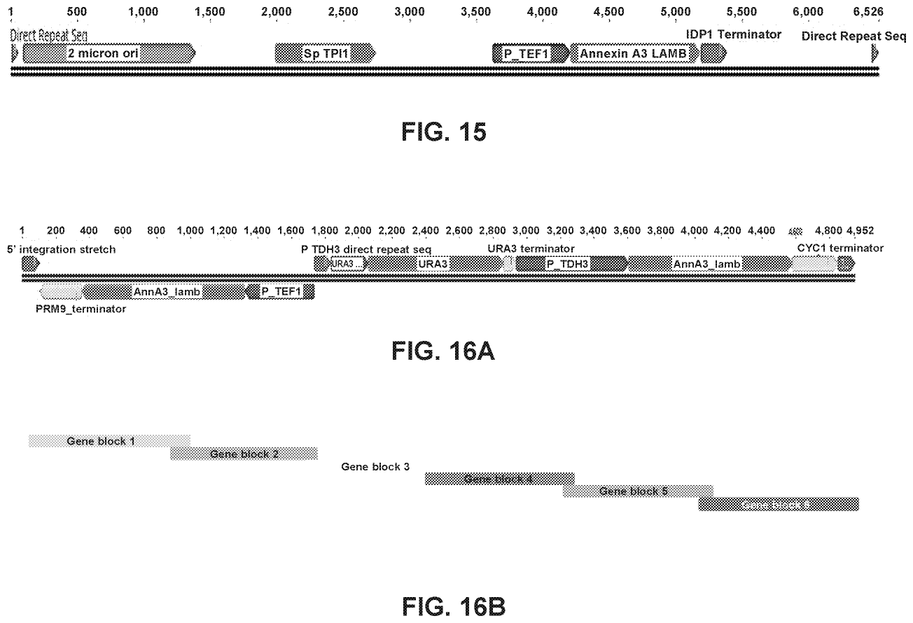

To introduce the expression vector into the S. cerevisiae strain S1 (MAT a tpi1 deletion::loxP), vector pBOND553 was first linearized with an SphI restriction digest. The resulting DNA fragments were separated by gel electrophoresis, and the large DNA fragment, containing the ANXA3 expression cassette (FIG. 15), was purified using the Zymoclean™ Gel DNA Recovery Kit. S1 was transformed with 1 μg of the purified linear DNA using the Frozen-EZ Yeast Transformation II Kit (Zymo Research, Cat #T2001). Transformants were selected by plating on synthetic complete agar media with glucose as the carbon source.

Deletion of the Sc TPI1 gene, indicated as tpi1 deletion or tpi1A, results in yeast cells that are not able to use glucose as a carbon source for growth. Expression of the S. pombe tpi1+ gene, which is present in the transformed linear DNA fragment, in a Sc tpi1 deletion strain rescues that defect restoring the ability of the yeast cells to grow on glucose media, and thus can be used to select for successful transformants (Compagno et al. 2001 and Carlsen et al. 1997).

The DNA transformed into the host strain (FIG. 15) has two homologous stretches of DNA sequence on either end of the linear fragment. Because the 50 bp homologous sequences are in the same orientation, the S. cerevisiae homologous recombination machinery is able to recombine the two DNA regions, leading to circularization of the linear fragment into a 2-micron high copy replicating plasmid. Successful circularization of the plasmid was verified by PCR using primers oBOND35 and oBOND47, which results in amplification of a 383 bp DNA fragment. Strain S2 was identified and tested for ANXA3 expression.

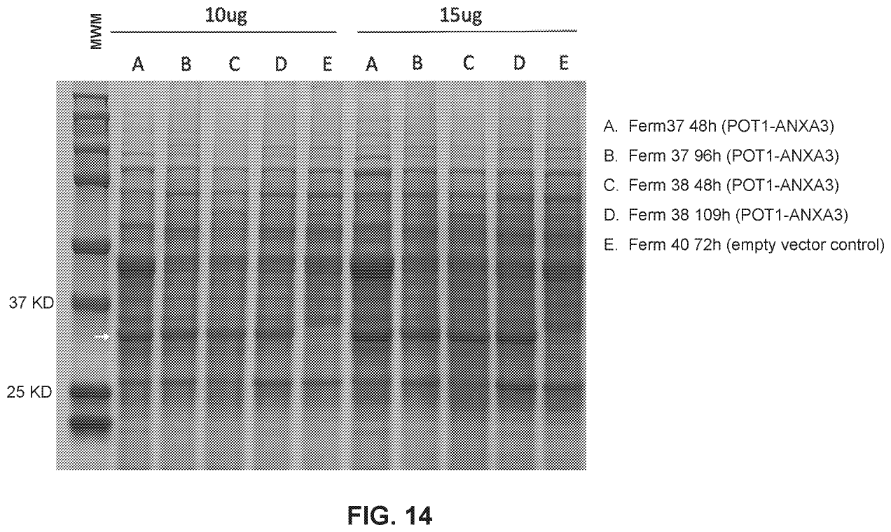

To analyze protein expression, the strain S2 was grown in a 3 L fermentation vessel in synthetically defined media with glucose for the carbon source. Cells were collected by centrifugation. Cell pellets with 1-2×10⁸ cells were collected, and protein extracts were prepared using a glass bead lysis protocol (Dunn, B. and Wobbe, C. R., 1993). Equal amounts of total protein were analyzed by protein gel electrophoresis and the proteins were visualized using Coomassie staining. FIG. 14 shows that strain S2 expresses high levels of ANXA3 protein compared to an empty vector control yeast strain.

References corresponding to Example 1, each of which are incorporated herein by reference in its entirety for all purposes: Carlsen M, Jochumsen K V, Emborg C, Nielsen J. Modeling the growth and proteinase A production in continuous cultures of recombinant Saccharomyces cerevisiae. Biotechnol Bioeng. 1997; Compagno C, Brambilla L, Capitanio D, Boschi F, Ranzi B M, Porro D. Alterations of the glucose metabolism in a triose phosphate isomerase-negative Saccharomyces cerevisiae mutant. Yeast. 2001; F. M. Ausubel, R. Brent, R. E. Kingston, D. D. Moore, J. G. Seidman, J. A. Smith, K. Struhl (Eds.), Current Protocols in Molecular Biology, John Wiley & Sons Inc, 2003; Dunn, B. and Wobbe, C. R. (1993), Preparation of Protein Extracts from Yeast. Current Protocols in Molecular Biology, 23: 13.13.1-13.13.9.

### Example 2

This example provides the use of a “safe harbor system” for the genomic integration of a lamb AnxA3 protein expression cassette in S. cerevisiae.

A full-length integration construct (FIG. 16A) was designed using DNA analysis software (Geneious Prime®, Dotmatics). This construct contains two AnnexinA3 (ANXA3) expression cassettes (Promoter—ANXA3 CDS—Terminator), in opposite orientation, with a URA3 selectable marker in between. The selectable marker is bracketed by two direct repeat homologous regions so that it can be excised by homologous recombination and recycled for another round of strain engineering, if so desired. Lastly, on both ends of the construct there are two regions of homology to a safe-harbor integration site in the S. cerevisiae genome to mediate integration of the construct by homologous recombination. Assembly of the expression construct, transformation into the host strain, and analysis of protein expression were done using well established molecular biology methods (Ausubel et al. 2003).

Safe-harbor integration sites are loci that have been shown to be regions of the genome with high levels of transcription (Wu et al. 2017), and where integration of the protein expression construct does not result in deleterious effects on the growth of the yeast strain.

To assemble the large integration construct, gene blocks spanning the entire sequence, each ~1 Kb long and overlapping with each other by ~100 bp (FIG. 16B), were ordered from Integrated DNA Technologies. Overlap extension PCR (SOE-PCR) (Zarghampoor et al. 2020) was used to stitch together the gene blocks into two separate fragments (5′ and 3′) used for transformation of the host strain. The two fragments split the URA3 marker into two pieces which overlap by ~150 bp. Recombination by S. cerevisiae between the overlapping regions of URA3 in the two fragments reconstitutes a functional URA3 thus providing the ability to select transformants.

SOE-PCR fragments were generated using oligos oBOND569 and oBOND574 for the 5′ fragment, and oligos oBOND575 and oBOND580 for the 3′ fragment. The PCR reactions were resolved by agarose gel electrophoresis and full length 5′ and 3′ fragments were purified using the Zymoclean™ Gel DNA Recovery Kit (Zymo Research, Cat #D4001). Equimolar amounts of the 5′ and 3′ fragments (~1 μg each fragment) were transformed into strain S4 (Mat a ura3-DBPF) using a lithium acetate transformation protocol (Becker, D. M. and Lundblad, V., 1994), and transformants were selected on synthetic complete agar media lacking uracil.

Correct transformants, with proper integration of the expression construct at the safe-harbor locus, were identified by diagnostic colony PCR amplification of the expected DNA sequence junctions that result when the construct integrates. Primers oBOND602 and oBOND595 were used to amplify the 5′ junction, and primers oBOND595 and oBOND603 were used to amplify the 3′ junction. Strains S5 (2xANXA3 cassette at the CIPI locus) and S6 (2xANXA3 cassette at the ADA2 locus) were identified and checked for ANXA3 expression.

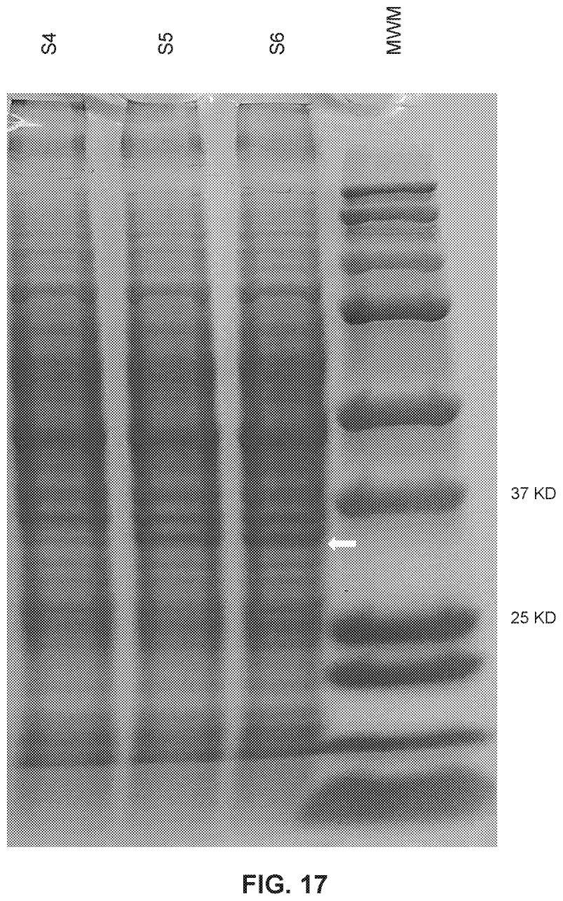

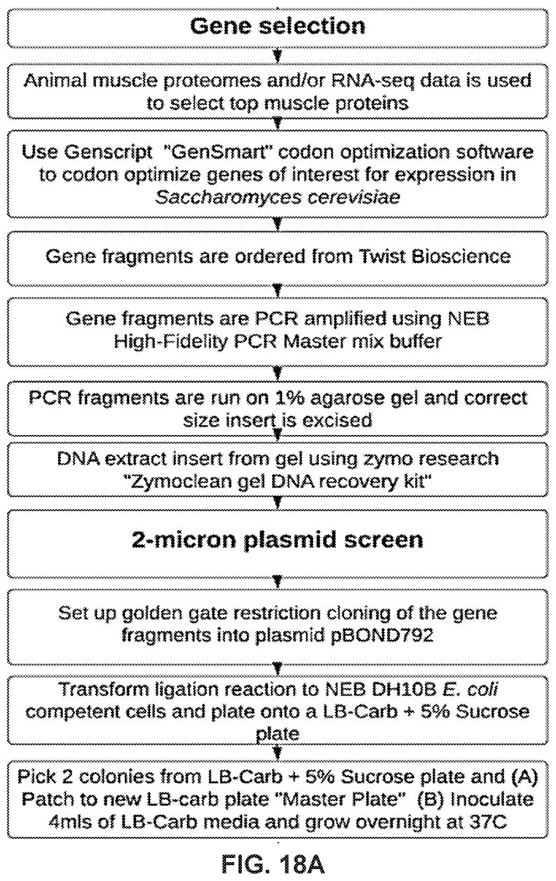

To analyze protein expression, strains S5 and S6, and the parental control strain S4, were grown to stationary phase in a shake flask with synthetic complete glucose media. Cell pellets with 1-2×10⁸ cells were collected by centrifugation, and protein extracts were prepared using a glass bead lysis protocol (Dunn, B. and Wobbe, C. R., 1993). Equal amounts of total protein were analyzed by protein gel electrophoresis and the proteins were visualized using Coomassie staining. FIG. 17 shows that strains S5 and S6 express ANXA3 protein (indicated by white arrow) compared to the parental control yeast strain.

References corresponding to Example 2, each of which are incorporated herein by reference in its entirety for all purposes: F. M. Ausubel, R. Brent, R. E. Kingston, D. D. Moore, J. G. Seidman, J. A. Smith, K. Struhl (Eds.), Current Protocols in Molecular Biology, John Wiley & Sons Inc, 2003; Wu X L, Li B Z, Zhang W Z, Song K, Qi H, Dai J B, Yuan Y J. Genome-wide landscape of position effects on heterogeneous gene expression in Saccharomyces cerevisiae. Biotechnol Biofuels. 2017; Zarghampoor F, Behzad-Behbahani A, Azarpira N, Khatami S R, Fanian M, Hossein Aghdaie M, Rafiei Dehbidi G. A Single Tube Overlap Extension PCR Method for Splicing of Multiple DNA Fragments. Avicenna J Med Biotechnol. 2020; Dunn, B. and Wobbe, C. R. (1993), Preparation of Protein Extracts from Yeast. Current Protocols in Molecular Biology, 23: 13.13.1-13.13.9; Becker, D. M. and Lundblad, V. (1994), Introduction of DNA into Yeast Cells. Current Protocols in Molecular Biology, 27: 13.7.1-13.7.10.

### Example 3

This example provides an exemplary search for annexin proteins which are useful for the methods and compositions disclosed herein.

A large number of animal annexins are known in the art. Any such animal annexins can be employed in the methods and compositions of the present disclosure. One of ordinary skill in the art can identify known annexins and isoforms thereof and obtain the amino acid sequences of such known annexins as well as the nucleic acid sequences of known annexin genes (including native regulatory sequences (e.g., promoters and terminators, thereof) from publicly available databases.

For example, the accession numbers for all annexins (as well as amino acid sequences and corresponding nucleic acids sequences associated with the annexins) in the NCBI database can be obtained. Protein accession numbers for all annexins in the NCBI database were retrieved using a custom Python script which utilized the following libraries:
requests: HTTP requests to the NCBI API.
xmltodict: Parse the XML response from the NCBI API into a Python dictionary.
pandas: Manage, analyze, and save the data in structured formats.
re: Extract specific patterns from the protein definitions.

The Python script performs the following steps:

1. Search term generation: Generate a list of plausible annexin names to search by according to the pattern ‘Annexin’+‘A-E’+‘1-20’. Resulting in the list [‘Annexin A1’, ‘Annexin A2’, . . . ‘Annexin E20’].

2. Unique identifier (UID) retrieval:

Construct a query to search the NCBI protein database:

$$query = f “ \{ name \} [ Protein Name ] ” .$$

Use the esearch endpoint of the NCBI E-utilities API to retrieve UIDs associated with each name:

$$search\_url = f “ https : // eutils . ncbi . nlm . nih . gov / entrez / eutils / esearch . fcgi ? db = protein \& term = \{ query \} \& retmax = 10000 ” response = requests . get ( search\_url )$$

Convert the XML response from the API into a Python dictionary:

$$data = xmltodict . parse ( response . content )$$

Store only unique UIDs to avoid duplicates.

3. Accession number and description retrieval:

Use summary endpoint of the NCBI E-utilities API to retrieve detailed information for each UID:

$$summary\_url = f “ \{ base\_url \} / esummary . fcgi ? db = protein \& id = \{ uid\_str \} ”$$

$$response = requests . get ( summary\_url )$$

$$data = xmltodict . parse ( response . content )$$

Extract relevant details such as the accession version, protein definition, and species from the XML response.

4. Data Management:

Convert the retrieved data into a pandas DataFrame

Save the DataFrame to a CSV file for further analysis and reference.

The outcome of running the Python script (September 2023) is a list of 15,138 unique accession numbers for annexin proteins and their isoforms. An exemplary subset of the Annexins retrieved in the search with accession numbers is provided in Table 2. Annexins of Table 2 are useful in the methods and food compositions of this disclosure.

### Example 4

This example provides an exemplary 2-micron plasmid screen performed prior to additional studies using the modified HapAmp method, the POT1 method, or the SafeHarbor method.

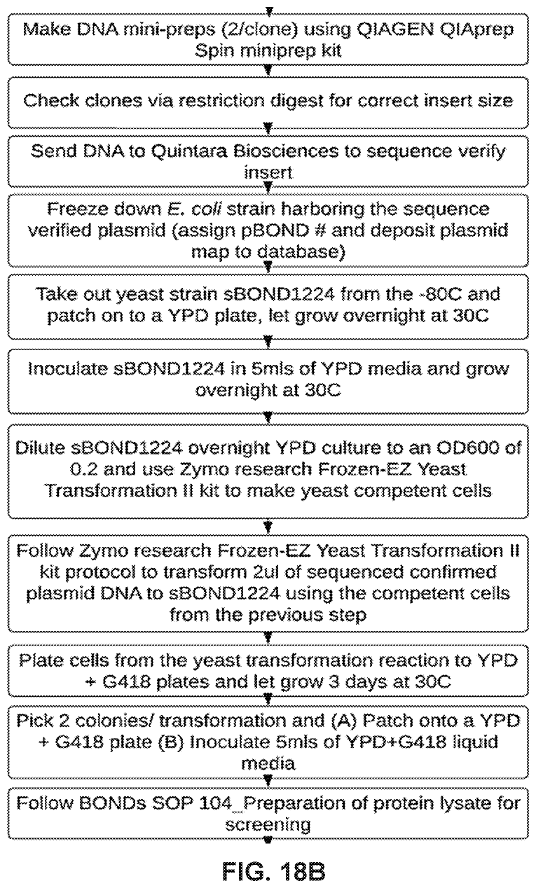

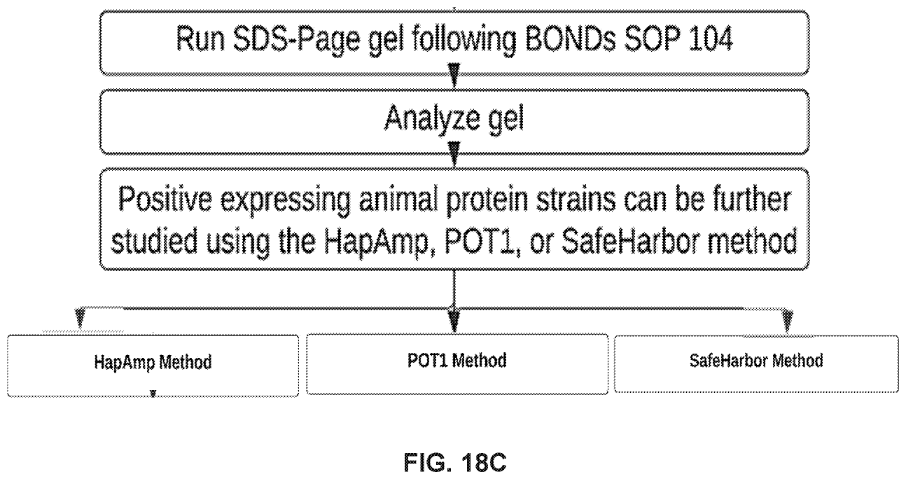

Gene selection is conducted as exemplified in Example 2, or by following the sequential steps outlined in FIGS. 18A, 18B, and 18C, in either case the resulting DNA fragments are extracted from the gel using the Zymoclean™ Gel DNA Recovery Kit (Zymo Research, Cat #D4001).

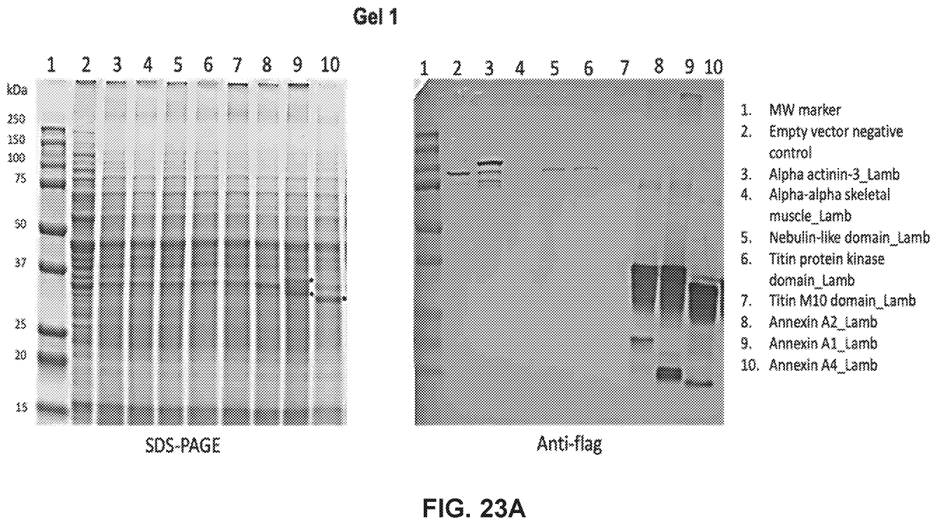

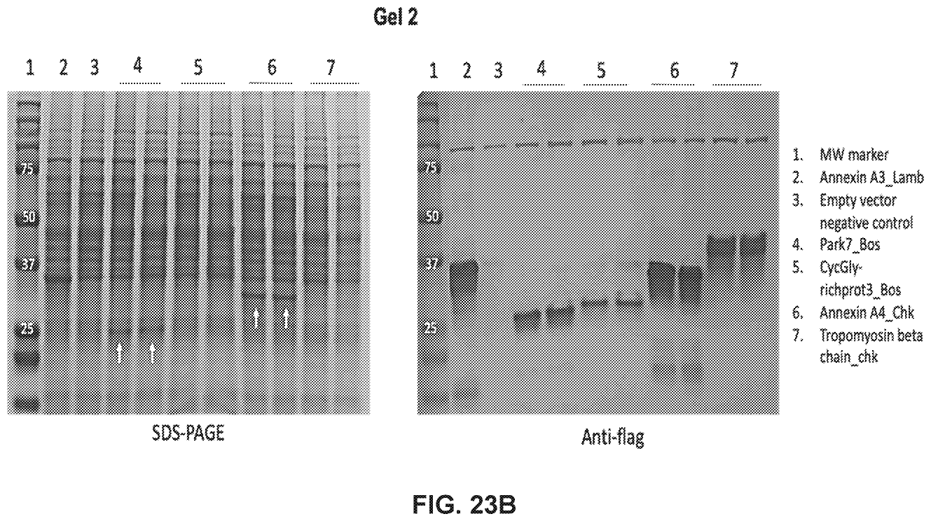

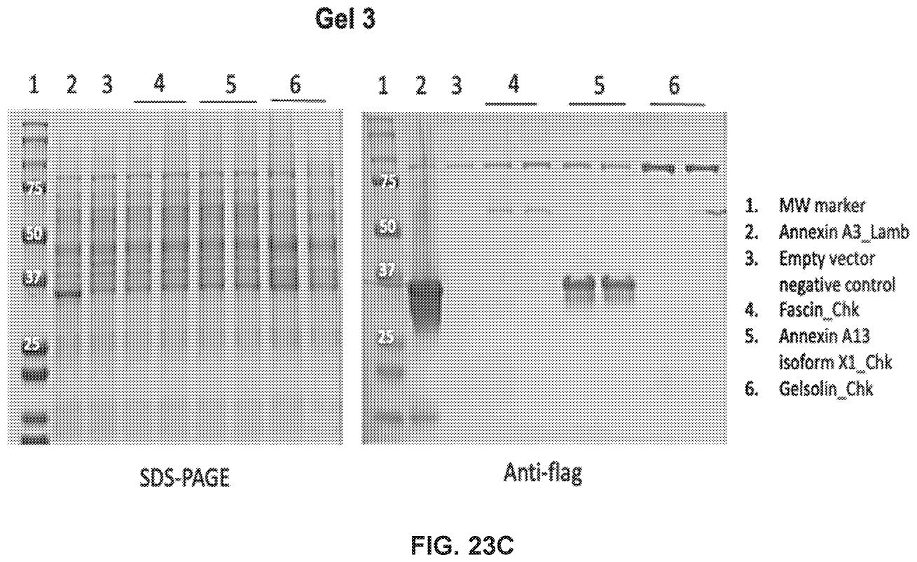

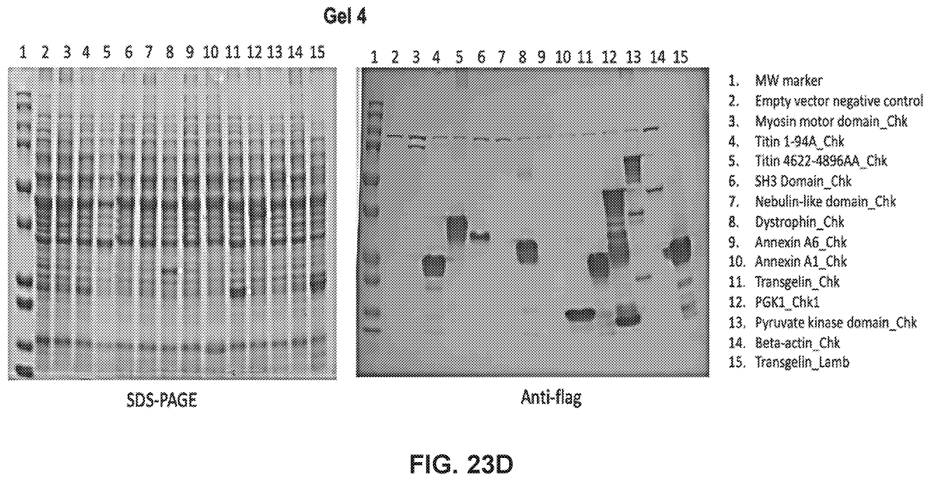

After gene selection, a 2-micron plasmid screen is performed by following the sequential steps of FIG. 18A-18C, producing results such as those depicted in FIGS. 23A-23D.

### Example 5

This example discusses an exemplary genetic construct design process for, and implementation of, a modified HapAmp method for transformation in Sc and subsequent in vivo amplification of a heterologous polypeptide.

In budding yeast (S. cerevisiae), genome-wide studies have previously investigated haploinsufficient growth phenotypes and demonstrated that expression dosage of haploinsufficient genes are closely tied to yeast growth fitness (Deutschbauer A M, et al. 2005). Among 5,900 yeast genes analyzed, approximately 3% (184 mutants) exhibited haploinsufficient growth in rich media (Deutschbauer A M, et al. 2005). Under limited nutrients, up to 20% showed haploinsufficient abnormalities (Delneri D, et al. 2008).

Many haploinsufficient genes are related to ribosomal assembly and function, suggesting a significant contribution of ribosomes to rapid growth (Deutschbauer A M, et al. 2005). Others are part of diverse metabolic processes like RNA transcription, proteosome function, glycolysis, and others (Deutschbauer A M, et al. 2005; Delneri D, et al. 2008).

Genes sensitive to haploinsufficiency revealed processes related to protein metabolism, including ribosome biosynthesis, rRNA processing, translation control, and protein folding (Deutschbauer A M, et al. 2005). Other haploinsufficient genes are involved in complex processes such as transcription from the RNA polymerase II, DNA replication, ER-to-Golgi transport, nuclear import/export, and cytoskeletal function (Deutschbauer A M, et al. 2005; Delneri D, et al. 2008).

The expression level of many haploinsufficient genes is tightly linked to growth fitness and cannot meet the needs for maximum growth when its dosage of normal product is reduced, then gene amplification, the increase in the number of gene copies, can occur through adaptive evolution to adjust the dosage of normal product needs.

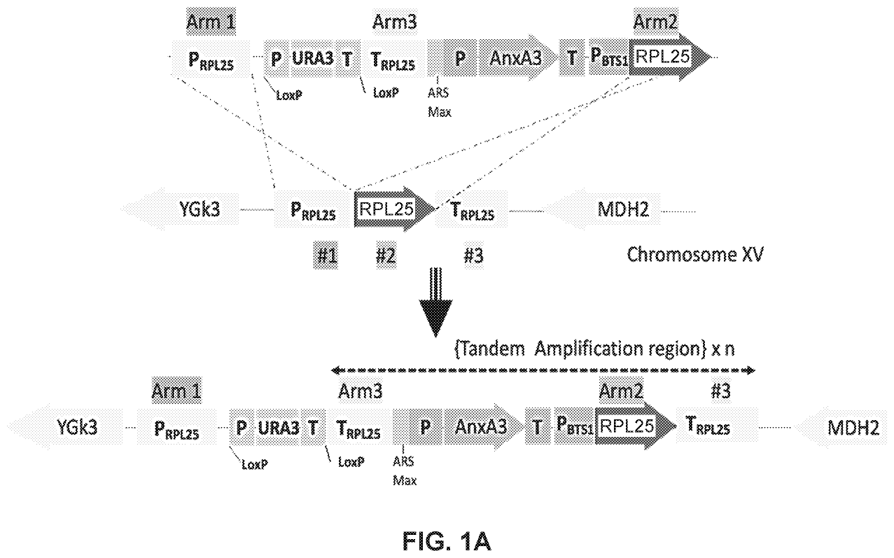

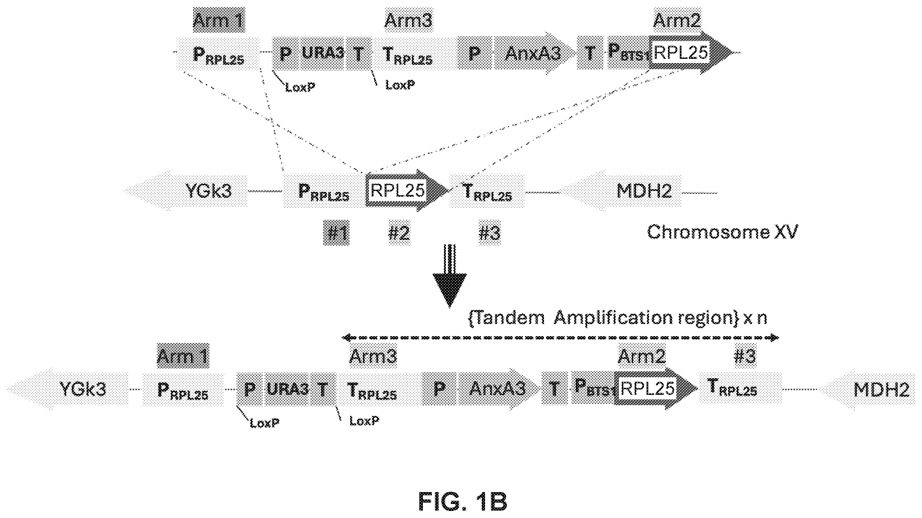

Gene amplification occurs naturally during cell proliferation and in laboratory experiments (Deutschbauer A M, et al. 2005; Schimke R T, et al. 1984). HapAmp is a method that uses haploinsufficiency as an evolutionary force to drive in vivo gene amplification in S. cerevisiae (Peng, et al. 2022). It has been demonstrated that artificial genetic structures based on haploinsufficient genes, like SEC23 and RPL25, enable gene amplification by tuning promoter strength or translational efficiency (e.g., by replacing the native promoter of a haploinsufficient gene with a much weaker one) (Peng, et al. 2022). These structures are incorporated into genetic vectors that can be used to introduce multiple copies of linked heterogeneous gene of interest (GOI). Integration occurs at the selected haploinsufficient locus in the yeast chromosome via homologous recombination, with notable results achieved when replacing the RPL25 promoter with the weaker BTS1 promoter (Peng, et al. 2022), such as exemplified in FIG. 1A and FIG. 1B.

References corresponding to Example 5, each of which are incorporated herein by reference in its entirety for all purposes: Cleary M A. Haploinsufficiency, Encyclopedia of Genetics (2001); Ohnuki S, and Ohya Y. High-dimensional single-cell phenotyping reveals extensive haploinsufficiency. PLoS Biology (2018); Deutschbauer A M, Jaramillo D F, Proctor M, Kumm J, Hillenmeyer M E, Davis R W, et al. Mechanisms of haploinsufficiency revealed by genome-wide profiling in yeast. Genetics (2005); Delneri D, Hoyle D C, Gkargkas K, Cross E J, Rash B, Zeef L, et al. Identification and characterization of high-flux-control genes of yeast through competition analyses in continuous cultures. Nature Genetics (2008); Schimke R T. Gene amplification in cultured animal-cells. Cell (1984); Peng B, Esquirol L, Lu Z, et al. An in vivo gene amplification system for high level expression in Saccharomyces cerevisiae. Nature Communications (2022); Sauer B. Functional expression of the cre-lox site-specific recombination system in the yeast Saccharomyces cerevisiae. Mol Cell Biol. (1987); Pir P., et al. The genetic control of growth rate: a systems biology study in yeast. BMC Syst Biol (2012)

### Example 6

This example provides an exemplary screen to enhance jackpot efficiency in modified HapAmp yeast transformations.

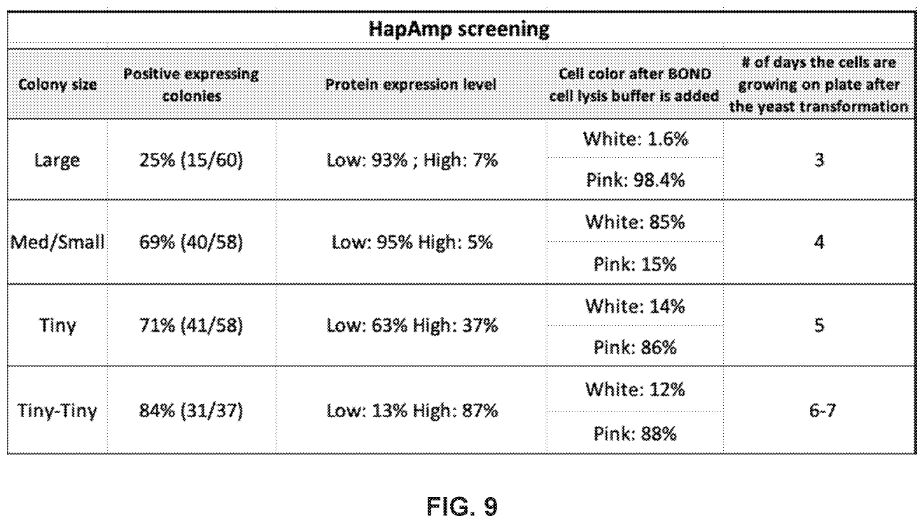

Preliminary results indicate that RPL25 HapAmp constructs with auxotrophic selectable marker URA3 achieve jackpot clone efficiency up to 20% of tested transformants. Interestingly, a detailed characterization of obtained HapAmp clones based on protein expression, growth, and colony size, resulted in an enrichment of jackpots from clones that exhibit slow growth and tiny colony size (FIG. 9). Colonies may be characterized by large, medium, small, tiny, or tiny-tiny size, where a large colony is 2.5 mm or greater in diameter, a medium colony is between 1.5 mm and 2.5 mm in diameter, a small colony is between 1 mm and 1.5 mm in diameter, a tiny colony is between 0.5 mm and 1 mm in diameter, and a tiny-tiny colony is less than 0.5 mm in diameter. We find that jackpots are enriched in transformation plates as clones that exhibit small colony size of less than 1.5 mm of diameter after 6 days of growth on synthetic complete agar media lacking uracil (YPD) at 30° C.

In some experiments, to boost jackpot efficiency for non-toxic proteins in Sc, additional parameters may be explored, such as (1) selective growth under nutrient-rich and nutrient-limited conditions. These include testing media with high and low magnesium, zinc, iron, copper, selenium, or vitamin B6 content. For instance, magnesium is essential for the proper assembly and function of ribosomes during protein synthesis, and/or (2) controlled drug treatments to induce DNA replication stress and affect ribosomal function during HapAmp transformation and recovery, using drugs like nocodazole, colchicine, paclitaxel, vinblastine, bleomycin, cycloheximide, hydroxyurea, gemcitabine, topoisomerase inhibitors (camptothecin, etoposide, doxorubicin), and polymerase (PARP) inhibitors.

### Example 7

This example provides modified HapAmp Screening of AnxA3:GFP and AnxA3 Jackpots.

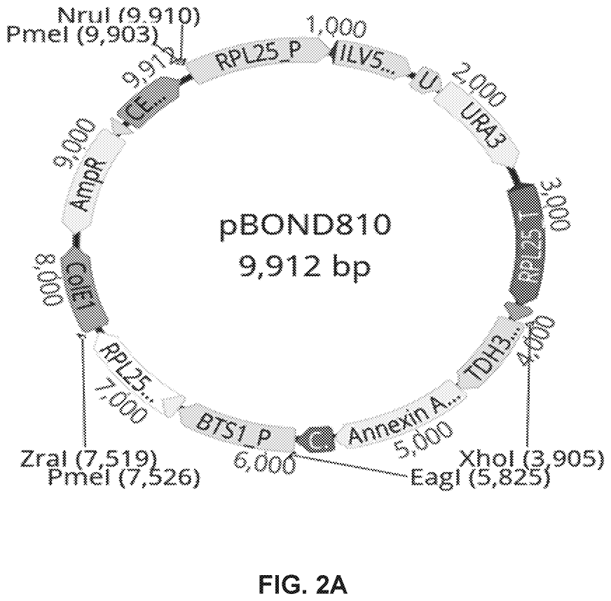

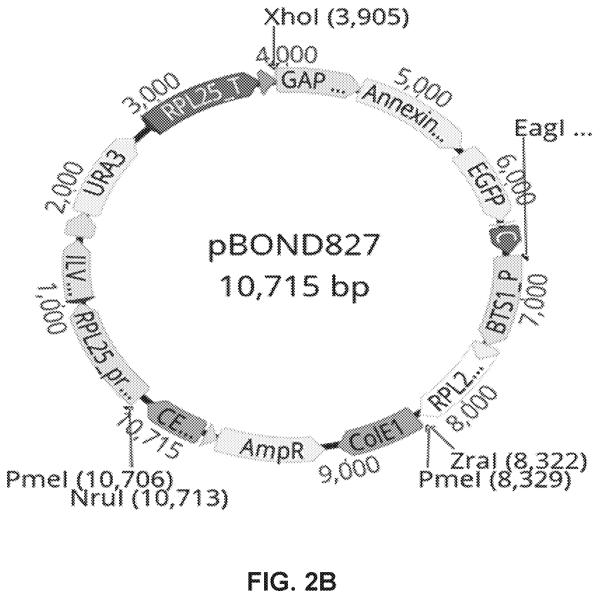

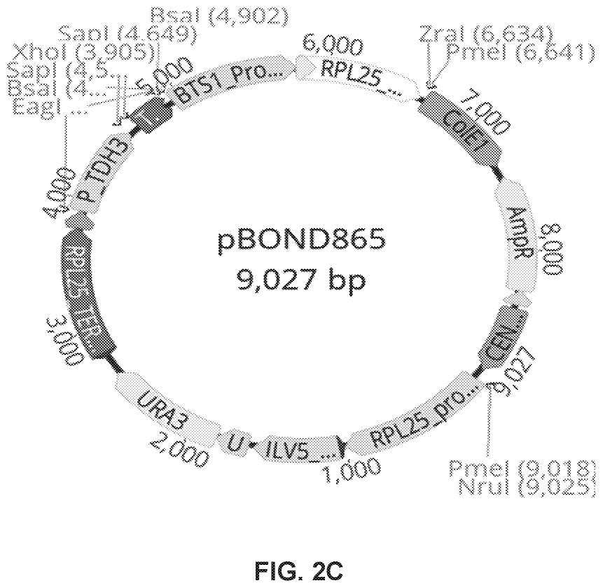

In this example, we utilize S. cerevisiae as a platform organism for producing yeast non-toxic heterologous animal proteins. We have developed a RPL25 HapAmp chromosomal integration plasmid, with the URA3 auxotrophic marker (FIG. 2). For HapAmp plasmid construction, RPL25 gene fragment CDS, promoter and terminator, were created as gBlocks™ (Integrated DNA Technologies) and assembled into 2-micron plasmid backbone by Gibson assembly.

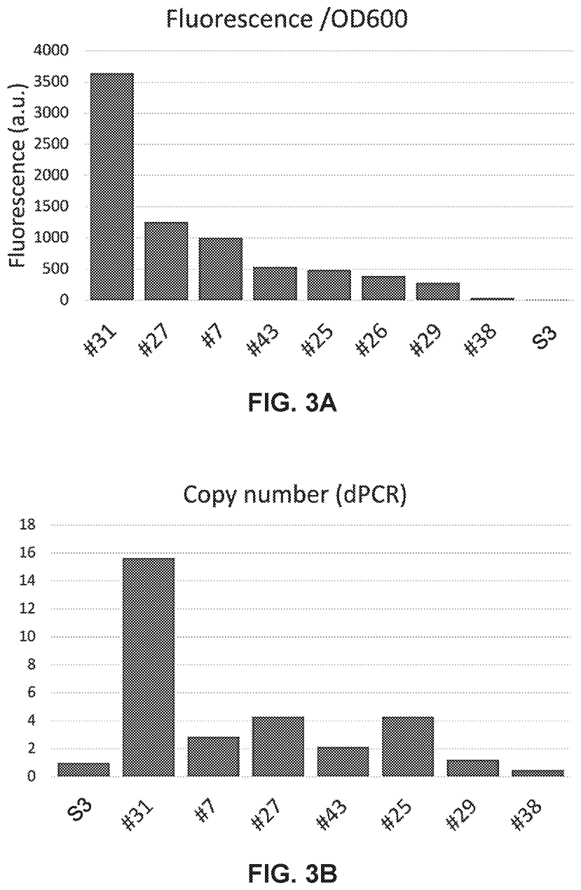

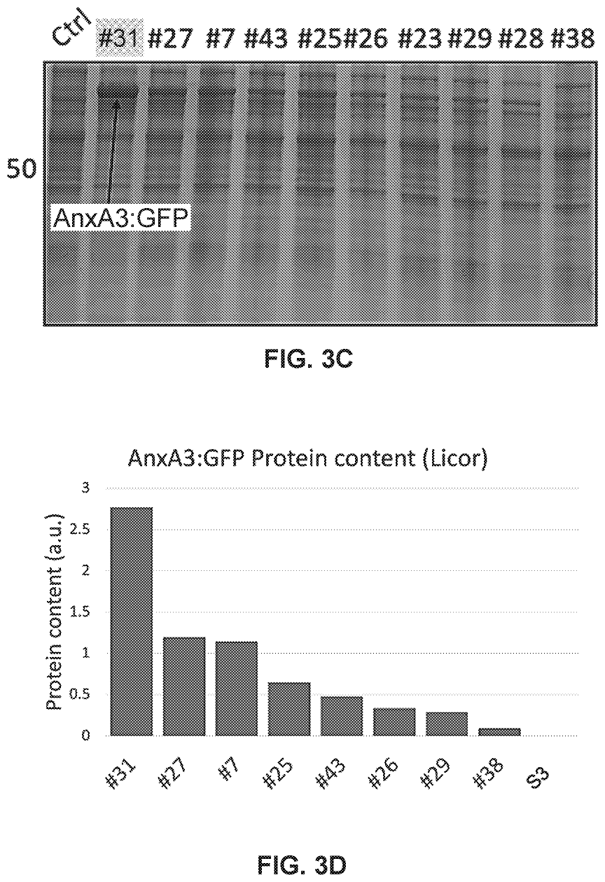

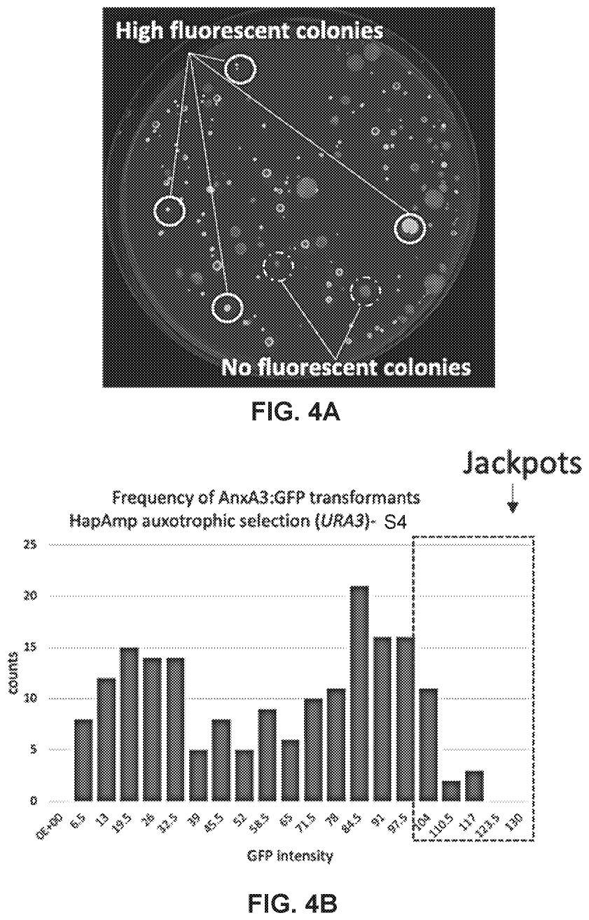

Unlike Peng et al. (2022), we have found that gene amplification is not solely dependent on adjusting promoter strength. In our transformation experiments with HapAmp RPL25 plasmids expressing fluorescent fusion protein: AnxA3:GFP, we have generated numerous HapAmp clones with varying fluorescence intensities (FIG. 3A) and copy number (FIG. 3B). Frequency histograms from obtained clones follow a bimodal distribution in which clones within the first mode lack or exhibit weak fluorescence-intensities, while clones within the second mode exhibit high fluorescence intensities (depicted in FIG. 4A and quantified in FIG. 4B). Furthermore, the study of selected clone pools reveals a strong correlation between fluorescence intensity and heterologous AnxA3:GFP protein content (depicted in FIG. 3C and quantified in FIG. 3D).

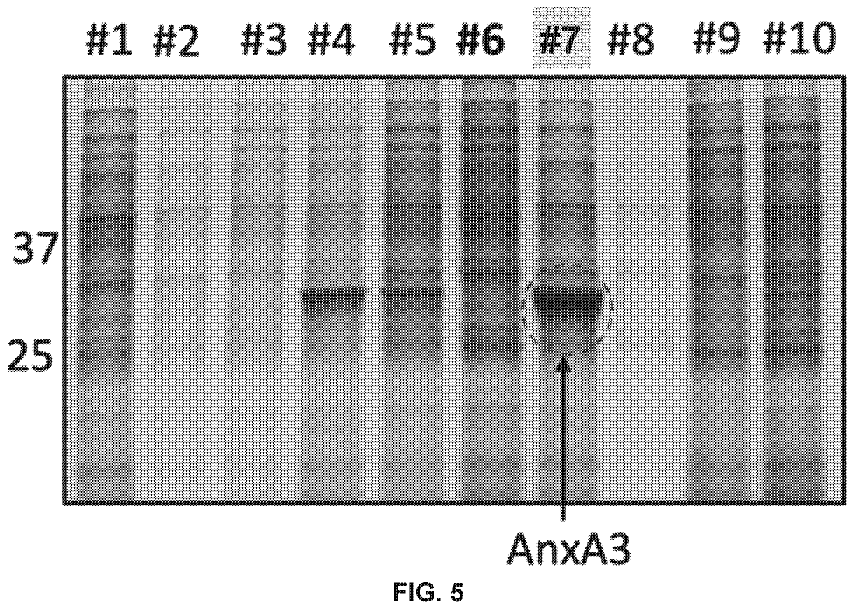

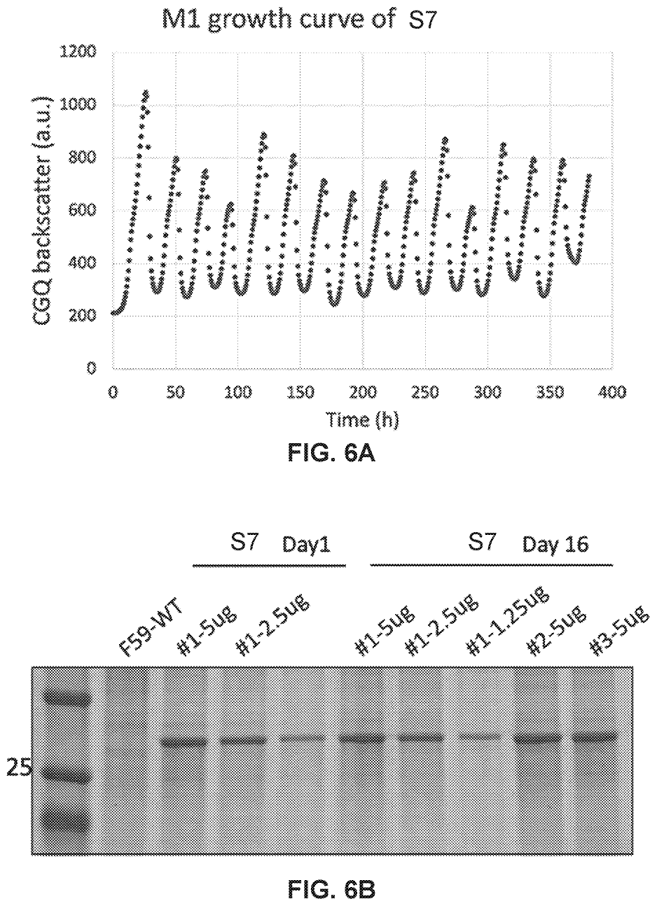

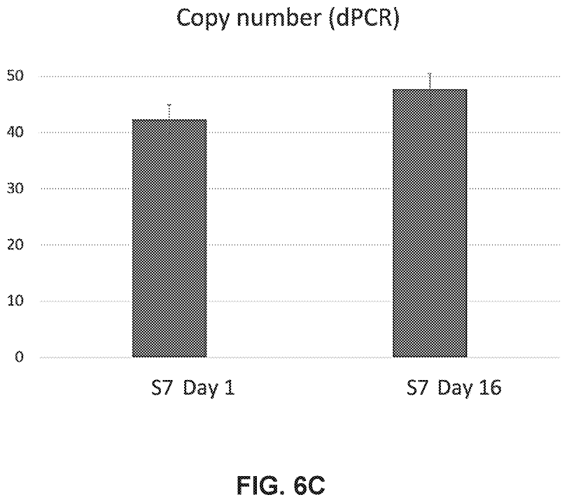

From the latter group, transformants that achieve robust expression without compromising cell growth are known as HapAmp JackLots, highly desirable outcomes. We have produced jackpots for AnnexinA3 (AnxA3), AnnexinA3:GFP, AnnexinA4 (AnxA4), AnnexinA5 (AnxA5), and Destrin. Jackpot protein content varies, ranging from 5% (e.g., Destrin) to up to 20% of total yeast peptides (e.g., AnnexinA3) as measured by tandem MS, wherein tandem MS specifically measured the detectable peptides (FIG. 3C and FIG. 5). Our data also demonstrate a strong correlation between the copy number of both the haploinsufficient gene and the gene of interest (GOI) and protein content in these transformants (FIG. 3 and FIG. 6). Furthermore, like Peng et al. (2022), we have shown that these transformants remain stably integrated at the selected haploinsufficient locus in the chromosome and exhibit robust protein expression for over 100 generations (FIG. 6).

References corresponding to Examples 7-9, each of which are incorporated herein by reference in its entirety for all purposes: Cleary M A. Haploinsufficiency, Encyclopedia of Genetics (2001); Ohnuki S, and Ohya Y. High-dimensional single-cell phenotyping reveals extensive haploinsufficiency. PLoS Biology (2018); Deutschbauer A M, Jaramillo D F, Proctor M, Kumm J, Hillenmeyer M E, Davis R W, et al. Mechanisms of haploinsufficiency revealed by genome-wide profiling in yeast. Genetics (2005); Delneri D, Hoyle D C, Gkargkas K, Cross E J, Rash B, Zeef L, et al. Identification and characterization of high-flux-control genes of yeast through competition analyses in continuous cultures. Nature Genetics (2008); Schimke R T. Gene amplification in cultured animal-cells. Cell (1984); Peng B, Esquirol L, Lu Z, et al. An in vivo gene amplification system for high level expression in Saccharomyces cerevisiae. Nature Communications (2022); Sauer B. Functional expression of the cre-lox site-specific recombination system in the yeast Saccharomyces cerevisiae. Mol Cell Biol. (1987); Pir P., et al. The genetic control of growth rate: a systems biology study in yeast. BMC Syst Biol (2012).

### Example 8

This example discusses developing new HapAmp Sites.

In addition to RPL25, we will engineer and assess multiple haploinsufficient loci for driving gene amplification. This will facilitate engineering S. cerevisiae strains expressing many animal proteins at multiple chromosomal integrations via multiple HapAmp sites. Genes sensitive to haploinsufficiency from various metabolic processes to be tested are depicted in Table 6, which is not an exhaustive list. We will assess their efficiency by driving the expression of fluorescent proteins. Top-performing HapAmp sites will be further engineered to boost the expression of the same or multiple genes, enhancing protein production in yeast. Selectable markers can be recycled using the Cre/loxP system (Sauer B., 1987), and additional auxotrophies (LEU2, HIS3, TRP1) will be introduced to streamline the process.

TABLE 6
Candidates for new HapAmp Sites.

| Protein(s) | Biological Process/Category |
|---|---|
| RPL17A, RPL18A, RPL33A | Protein Metabolism - Large ribosome |
| | subunit |
| RPS15, RPS20 | Protein Metabolism - Small ribosome |
| | subunit |
| CCT2, CCT4, CCT6 | CCT Folding Chaperone - Protein |
| | Metabolism |
| RPB3, RPB4, RPB5, RPB7, | Transcription from the RNA Polymerase II |
| RPB8, SRB7, RPO26, RPC10 | |
| RPN11 | Supports Proteasome Activity |
| MCM2, CDC47 | DNA Replication |
| SUI2 | Translation Initiation Factors |
| RVB2 | Transcriptional Regulation |
| SEC23, SEC34 | ER-to-Golgi Transport |
| NUP60, NUP145, NUP120, | Nuclear Import and/or Export |
| NIC96 | |
| ACT1, TUB1, SPC97, SPC98 | Cytoskeletal Function |
| TDH3, HXK1, GPM1, ENO1, | Glycolysis |
| TPI1, CDC19, PDC2, ADH3 | |

### Example 9

This example discusses the HARES and HARESCO methods.

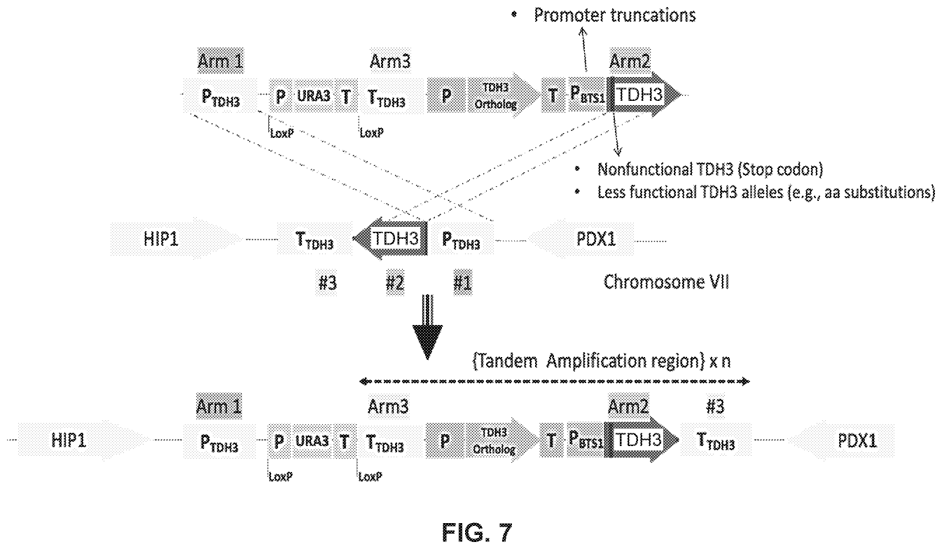

HARES is a novel HARES is a novel method to boost protein expression in yeast by combining gene amplification of haploinsufficient genes with the rescue of yeast's loss-of-function genes using animal orthologs (FIG. 7). It can be applied to any biological processes involving haploinsufficient genes as listed before. For instance, we are testing HARES on the key gene, glyceraldehyde-3-phosphate dehydrogenase (TDH3, GAPDH), in the yeast glycolytic pathway. In HARES, we create loss-of-function mutations of the targeted haploinsufficient gene while enhancing expression and rescue through animal orthologs via gene amplification. Loss-of-function mutations can be achieved by either reducing the activity of the native yeast gene with less functional allele variants or by abolishing its activity with stop codons. Gene amplification not only increases the copy number of the native gene but also the ortholog one, which can rescue the function of the mutated yeast gene and enhance growth fitness.

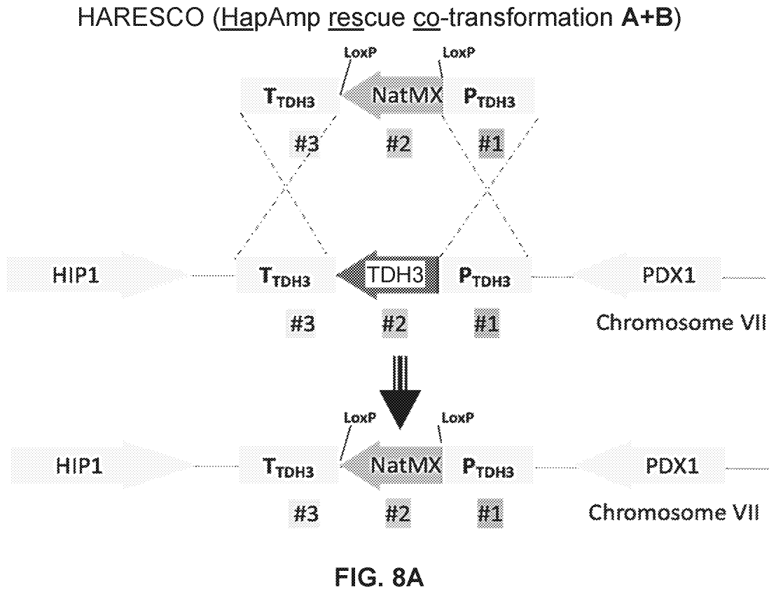

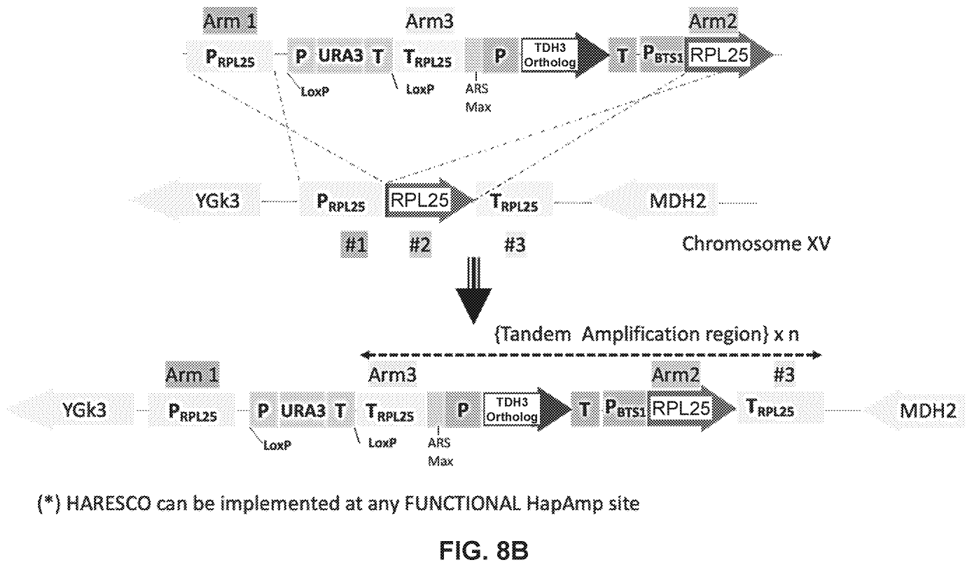

An additional modified method from HARES named HARESCO, for HapAmp rescue co-transformation, is described in FIG. 8. It is based on co-transformation experiment in S. cerevisiae by combining: (A) specific targeted deletion of gene of interest (GOI) (e.g., TDH3) by homology recombination with a recyclable selectable marker (e.g., G-418), and (B) HapAmp RPL25 for gene amplification of the targeted gene animal ortholog. HARESCO can be implemented at any functional HapAmp site.

### Example 10

This example discusses copy number analysis of strains.

Gene copy number was estimated through whole-genome sequencing and analysis of mapped read counts at the integration sites. Genomic DNA from transformed strains was sequenced using an Illumina NovaSeq X Plus, generating 2×151 bp paired-end reads with an average yield of 1×10⁹ total bp per sample. Demultiplexing, quality control, and adapter trimming were performed using bcl-convert (v4.2.4). Paired reads were then aligned to custom reference genomes, each containing a single copy of the recombinant DNA at the expected integration site, using HISAT2 (v2.2.1). Mapped reads were quantified using featureCounts (v2.0.3). Finally, the gene copy number was estimated by comparing the average read count of unique genes within the amplified region of the HapAmp locus to ten adjacent, non-amplified genes.

Copy number analysis was performed on strains S8, S21, and S25 which contain genes coding for different annexins and a fatty acid-binding protein across RPL25 and RPL33 sites.

Strain S18 contains an estimated 17 copies of annexin A4 in the RPL25 site and 19 copies each of annexin A1 and annexin A4 in the RPL33 site. S21 contains an estimated 34 copies each of annexin A4 and fatty-acid binding protein 4 in the RPL25 site and 7 copies each of annexin A3 and annexin A8 in the RPL33 site. S26 contains an estimated 31 copies each of annexin A4 and fatty-acid binding protein 4 in the RPL25 site and 18 copies each of annexin A1 and annexin A4 in the RPL33 site.

### Example 11

This example provides the compositional analysis of certain developed strains, S7, S9, and S25 as compared to the compositions of unengineered strain, S3. The results of which are provided in Tables 7 and 8 below. As noted above, each of strains S7, S9, and S25 were transformed using the modified HapAmp transformation method.

TABLE 7
Amino acid content and crude protein content in un-engineered strain (S3) and engineered strains (S7, S9, and S25).

| | S3 | | S7 | | S9 | | S25 | |
|---|---|---|---|---|---|---|---|---|
| % Component/ | Concentration | | Concentration | | Concentration | | Concentration | |
| Dry Mass | (%) (N = 3) | STDEV | (%) (N = 16) | STDEV | (%) S9 (N = 28) | STDEV | (%) (N = 2) | STDEV |
| Aspartic acid | 4.60 | 0.58 | 5.86 | 0.42 | 4.87 | 0.68 | 5.90 | 0.38 |
| Threonine | 1.98 | 0.23 | 2.60 | 0.51 | 2.11 | 0.38 | 2.76 | 0.17 |
| Serine | 1.97 | 0.65 | 2.70 | 0.66 | 2.14 | 0.27 | 2.53 | 0.08 |
| Glutamic acid | 5.95 | 0.87 | 7.52 | 0.74 | 6.45 | 1.10 | 6.91 | 0.78 |
| Proline | 1.72 | 0.10 | 1.61 | 0.16 | 1.51 | 0.15 | 1.69 | 0.04 |
| Glycine | 2.17 | 0.20 | 2.70 | 0.29 | 2.06 | 0.31 | 2.49 | 0.15 |
| Alanine | 2.81 | 0.46 | 3.52 | 0.47 | 3.13 | 0.43 | 3.40 | 0.31 |
| Cystine | 0.44 | 0.03 | 0.54 | 0.10 | 0.48 | 0.16 | 0.43 | 0.04 |
| Valine | 2.45 | 0.23 | 2.68 | 0.78 | 2.63 | 1.49 | 3.05 | 0.34 |
| Methionine | 0.59 | 0.02 | 0.76 | 0.05 | 0.86 | 0.09 | 0.83 | 0.04 |
| Isoleucine | 2.08 | 0.27 | 2.83 | 0.44 | 2.17 | 0.36 | 2.35 | 0.38 |
| Leucine | 3.00 | 0.06 | 4.39 | 0.41 | 3.22 | 0.50 | 3.67 | 0.37 |
| Tyrosine | 1.83 | 0.08 | 1.95 | 0.21 | 1.79 | 0.14 | 2.13 | 0.15 |
| Phenylalanine | 2.13 | 0.17 | 2.15 | 0.19 | 1.83 | 0.23 | 2.32 | 0.14 |
| Lysine (total) | 3.33 | 0.11 | 4.44 | 0.49 | 3.45 | 0.66 | 4.02 | 0.56 |
| Histidine | 1.08 | 0.06 | 0.98 | 0.11 | 0.88 | 0.11 | 0.97 | 0.08 |
| Arginine | 2.32 | 0.51 | 2.53 | 0.44 | 2.55 | 0.52 | 2.49 | 0.38 |
| Tryptophan | 0.51 | 0.06 | 0.52 | 0.03 | 0.53 | 0.10 | 0.59 | 0.05 |
| Crude Protein | 52.4^{‡} | 3.81 | 59.7^{†} | 8.12 | 53.3* | 4.48 | 57.30 | 4.46 |
| (Total Kjeldahl | | | | | | | | |
| Protein) | | | | | | | | |

*N = 50; ^{†}N = 6; ^{‡}N = 2

TABLE 8
Proximates and mineral content in unengineered strain (S3) and engineered strains (S7, S9, and S25). NA = Not applicable.

| | | S3 | | S7 | | S9 | | S25 | |
|---|---|---|---|---|---|---|---|---|---|
| | | Concentration | | Concentration | | Concentration | | Concentration | |
| Analyte | Units | (N = 4) | STDEV | (N = 18) | STDEV | (N = 49) | STDEV | (N = 2) | STDEV |
| Fat (acid | % | 6.0* | 0.38 | 4.91^{†} | 1.53 | 5.73^{‡} | 1.02 | 5.54 | 0.21 |
| hydrolysis) | | | | | | | | | |
| Fat (crude) | % | 1.1 | 0.55 | 1.46 | 0.93 | 1.16^{‡} | 0.51 | 2.10 | 0.42 |
| Fiber (crude) | % | 2.7 | NA | 0.79 | 0.55 | 0.59^{‡} | 0.32 | <0.20 | NA |
| Ash | % | 5.6 | 0.47 | 5.70 | 0.77 | 6.28^{‡} | 1.02 | 6.98 | 0.05 |
| Sulfur (total) | % | 0.37 | 0.05 | 0.43 | 0.12 | 0.54 | 0.14 | 0.63 | 0.03 |
| Phosphorus | % | 1.50 | 0.29 | 1.33 | 0.22 | 1.54 | 0.19 | 1.49 | 0.07 |
| (total) | | | | | | | | | |
| Potassium | % | 1.84 | 0.31 | 1.57 | 0.23 | 1.75 | 0.36 | 1.82 | 0.08 |
| (total) | | | | | | | | | |
| Magnesium | % | 0.20 | 0.01 | 0.17 | 0.02 | 0.19 | 0.03 | 0.15 | 0.00 |
| (total) | | | | | | | | | |
| Calcium | % | 0.03 | 0.01 | 0.02 | 0.01 | 0.02 | 0.01 | 0.04 | 0.01 |
| (total) | | | | | | | | | |
| Sodium | % | <0.01 | NA | 0.01 | 0.00 | <0.01 | NA | <0.01 | NA |
| (total) | | | | | | | | | |
| Iron (total) | ppm | 367 | 47 | 277^{†} | 31.1 | 356 | 153 | 266 | 9.19 |
| Manganese | ppm | 26.4 | 2.5 | 29.8^{†} | 8.9 | 25.7 | 16.5 | 37.1 | 1.13 |
| (total) | | | | | | | | | |
| Copper | ppm | 3.40 | 1.2 | 3.3^{†} | 1.0 | 3.76 | 1.72 | 3.35 | 0.64 |
| (total) | | | | | | | | | |
| Zinc (total) | ppm | 210 | 66 | 160 | 56.7 | 206 | 64.5 | 238 | 11.3 |

*N = 2; ^{†}N = 14; ^{‡}N = 48

### Example 12

This example provides the compositional analysis of certain developed strains, S7 and S9, as compared to the compositions of common pet food ingredients known in the art. The results of which are provided in Table 9 below.

TABLE 9
Compositional analysis of inactivated, dried S. cerevisiae expressing an animal protein compared to common pet
food ingredients, such as animal meal. ND = not disclosed. NT = Not Tested. N refers to the number of replicates.
In the cases where a particular assay has less or more than the listed number of replicates, it is noted below the table.

| | | Strain | Strain | Chicken | Poultry by- | Meat | Meat meal |
|---|---|---|---|---|---|---|---|
| | | S7(Average, | S9(Average, | by-product | product | meal, | with bone, |
| Analyte | Unit | N = 3) | N = 3) | mealⁱ | mealⁱⁱ | renderedⁱⁱ | renderedⁱⁱ |
| Moisture | % | 5.6 | 5.3 | 5.36 | 6.50 | 6.10 | 6.00 |
| Protein Crude | % | 54 | 51 | 67.66 | 59.00 | 54.10 | 50.90 |
| (Kjeldahl) | | | | | | | |
| Ash | % | 5.7 | 5.9 | 10.54 | 16.00 | 21.80 | 19.20 |
| Fat, crude | % | 5.1 | 4.8 | 13.56 | 13.50 | 11.80 | 9.80 |
| Fiber, crude | % | <0.02 | 0.3 | 1.04 | 2.00 | 2.50 | 2.80 |
| Metabolizable | kcal/kg | 4326 | 3356 | ND | ND | ND | ND |
| Energy | | | | | | | |
| Total dietary | % | 27.7 | 29.8 | ND | ND | ND | ND |
| fiber | | | | | | | |
| Insoluble | % | 24.6 | 27.2 | ND | ND | ND | ND |
| dietary fiber | | | | | | | |
| Soluble dietary | % | 3.1 | 2.5 | ND | ND | ND | ND |
| fiber | | | | | | | |
| Alanine | % | 3.2 | 3.0 | ND | ND | ND | ND |
| Arginine | % | 3.0 | 3.1 | 4.8 | 3.89 | 3.82 | 3.55 |
| Asparagine | % | 5.3 | 5.1 | ND | ND | ND | ND |
| Aspartic Acid | % | 0.48 | 0.53 | 0.95 | ND | ND | ND |
| Cystine | % | 6.7 | 6.5 | ND | ND | ND | ND |
| Glutamic Acid | % | 2.4 | 2.2 | 6.07 | ND | ND | ND |
| Glutamine | % | 1.0 | 0.9 | 1.41 | 1.34 | 1.11 | 0.96 |
| Glycine | % | 3.0 | 2.5 | 2.79 | 2.25 | 1.60 | 1.41 |
| Histidine | % | 4.5 | 3.8 | 5.1 | 4.20 | 3.41 | 3.12 |
| Isoleucine | % | 4.4 | 3.8 | 4.59 | 2.84 | 2.91 | 2.64 |
| Leucine | % | 0.81 | 1.02 | 1.38 | 1.02 | 0.77 | 0.71 |
| Lysine | % | 2.3 | 2.1 | 2.83 | 2.04 | 1.93 | 1.71 |
| Methionine | % | 1.5 | 1.4 | 4.13 | ND | ND | ND |
| Phenylalanine | % | 2.5 | 2.4 | 2.62 | ND | ND | ND |
| Proline | % | 2.7 | 2.5 | 2.76 | 2.10 | 1.83 | 1.67 |
| Serine | % | 0.60 | 0.57 | 0.73 | 0.46 | 0.36 | 0.30 |
| Threonine | % | 1.6 | 1.7 | 2.44 | 1.68 | ND | 1.20 |
| Tryptophan | % | 2.6 | 2.8 | 3.39 | 2.76 | 2.40 | 2.14 |
| Tyrosine | ppm | <100 | ND | 2.44 | 0.31 | ND | ND |
| Valine | % | 0.013 | 0.012 | ND | 3.50 | 8.31 | 9.97 |
| Taurine | % | 0.070 | 0.133 | ND | 0.55 | 0.66 | 0.51 |
| Calcium | % | 0.17 | 0.16 | ND | 0.21 | 0.25 | 0.24 |
| Chloride | % | 1.2 | 1.2 | ND | 2.05 | 3.94 | 4.46 |
| Magnesium | % | 1.3 | 1.4 | ND | 0.58 | 0.46 | 0.97 |
| Phosphorus | % | 0.030 | 0.143 | ND | 0.35 | 0.75 | 0.67 |
| Potassium | % | 0 | 0.45 | ND | 0.51 | 0.48 | 0.38 |
| Sodium | ppm | 7 | <5 | ND | ND | ND | ND |
| Sulfur | ppm | 3 | 3.0 | ND | 14.00 | 19.56 | 9.26 |
| Aluminum | ppm | 5.9 | 5.4 | ND | ND | ND | ND |
| Copper | ppm | 276 | 281 | ND | 470.00 | 655.00 | 564.00 |
| lodine | ppm | 23 | 26 | ND | 11.00 | 23.00 | 21.00 |
| Iron | ppm | <0.05 | <0.05 | ND | 0.78 | 0.42 | 0.25 |
| Manganese | ppm | 129 | 140 | ND | 120.00 | 106.00 | 88.00 |
| Molybdenum | ppm | <0.10 | 0.013 | ND | ND | ND | ND |
| Selenium | ppm | 0.22 | 0.368* | ND | ND | ND | ND |
| Zinc | ppm | <0.020 | <0.01 | ND | ND | ND | ND |
| Arsenic | ppm | <0.50 | <0.5 | ND | ND | ND | ND |
| Chromium | ppm | <0.10 | <0.01 | ND | ND | ND | ND |
| Cadmium | ppm | <0.01 | <0.01 | ND | ND | ND | ND |
| Cobalt | ppm | 26 | 70 | ND | ND | ND | ND |
| Lead | ppm | <5 | <1.0 | ND | ND | ND | ND |
| Mercury | % | <0.0015 | <0.0015 | ND | ND | ND | ND |
| Nitrate | % | <0.06 | NT | ND | ND | ND | ND |
| Nitrite (as N) | % | 0.47 | NT | ND | ND | ND | ND |
| Fluoride | % | <0.05 | <0.05 | ND | ND | ND | ND |
| | (w/w) | | | | | | |
| Hydroxyproline | % | <0.05 | <0.05 | ND | ND | ND | ND |
| | (w/w) | | | | | | |
| Sulfate | % | <0.02 | <0.02 | ND | ND | ND | ND |
| | (w/w) | | | | | | |
| Omega 3 Total | % | <0.02 | <0.02 | ND | ND | ND | ND |
| | (w/w) | | | | | | |
| Omega 6 Total | % | <0.02 | <0.02 | ND | ND | ND | ND |
| | (w/w) | | | | | | |
| C18:2 Omega 6 | % | | | ND | ND | ND | ND |
| (Linoleic) | (w/w) | | | | | | |
| C18:3 Omega 3 | % | | | ND | ND | ND | ND |
| (alpha- | (w/w) | | | | | | |
| Linolenic) | | | | | | | |
| C20:4 | ppm | <0.300 | <0.3 | ND | ND | ND | ND |
| Arachidonic (all | | | | | | | |
| isomers) | | | | | | | |
| C16:0 | ppm | <0.180 | <0.18 | ND | ND | ND | ND |
| Hexadecanoic | | | | | | | |
| (Palmitic) | | | | | | | |
| C16:1 | ppm | 35.2 | ND | ND | 1 | 0.2 | 0.8 |
| Hexadecenoic | | | | | | | |
| (Palmitoleic) | | | | | | | |
| C18:0 | ppm | 40.3 | 36.0 | ND | ND | ND | ND |
| Octadecanoic | | | | | | | |
| (Stearic) | | | | | | | |
| C18:1 | ppm | 47.9 | 44.8 | ND | 10.5 | 5.2 | 4.4 |
| Octadecenoic | | | | | | | |
| (Oleic + isomers) | | | | | | | |
| DHA (22:6) | ppm | 548.3 | 574.0 | ND | 40 | 59 | 46 |
| EPA (20:5) | ppm | 20.8 | 27.1 | ND | 10.6 | 5.8 | 4.1 |
| Vitamin A - | ppm | 32.7 | 38.8 | ND | 4.4 | ND | 12.8 |
| Beta Carotene | | | | | | | |
| Vitamin A - | ppm | 0.5 | 0.5 | ND | 0.3 | 0.14 | 0.14 |
| Retinol | | | | | | | |
| Vitamin B1 - | ppm | 21.7 | 15.5 | ND | 1 | 0.6 | 0.30 |
| Thiamin Base | | | | | | | |
| Vitamin B1- | ppm | <0.0044 | <0.0044 | ND | 0.31 | 0.09 | 70 |
| Thiamine | | | | | | | |
| Hydrochloride | | | | | | | |
| Vitamin B2- | IU/ | <40 | <50 | ND | ND | ND | ND |
| Riboflavin | kg | | | | | | |
| Vitamin B3- | IU/ | <5 | <1.97 | ND | 3.1 | ND | 1.49 |
| Niacin | kg | | | | | | |
| Vitamin B5- | ppm | 2763 | 3000 | ND | 5966 | 2200 | 1996 |
| Pantothenic | | | | | | | |
| Acid | | | | | | | |
| Vitamin B6- | % | 5.6 | 5.3 | 5.36 | 6.50 | 6.10 | 6.00 |
| Pyridoxine | | | | | | | |
| Vitamin B7- | % | 54 | 51 | 67.66 | 59.00 | 54.10 | 50.90 |
| Biotin | | | | | | | |
| Vitamin B9- | % | 5.7 | 5.9 | 10.54 | 16.00 | 21.80 | 19.20 |
| Folic Acid | | | | | | | |
| Vitamin B12- | % | 5.1 | 4.8 | 13.56 | 13.50 | 11.80 | 9.80 |
| Cobalamin | | | | | | | |
| Vitamin D | % | <0.02 | 0.3 | 1.04 | 2.00 | 2.50 | 2.80 |
| Vitamin E | kcal/kg | 4326 | 3356 | ND | ND | ND | ND |
| Choline | % | 27.7 | 29.8 | ND | ND | ND | ND |
| | | Chicken | | | | | |
| | | Meal, Low | Beef | Salmon | Lamb | Turkey | Pork |
| Analyte | Unit | Ash^{iiu} | Meal^{iiu} | Meal^{iiu} | Mealⁱⁱⁱ | Mealⁱⁱⁱ | Mealⁱⁱⁱ |
| Moisture | % | 5.65 | 5.208 | 7.44 | 3.52 | 5.23 | 3.67 |
| Protein Crude | % | 94.35 | 94.78 | 92.56 | 96.48 | 94.77 | 96.33 |
| (Kjeldahl) | | | | | | | |
| Ash | % | 14 | 31.02 | 19.12 | 25.1 | 18 | 23.8 |
| Fat, crude | % | 12 | 10.68 | 12.5 | 12.3 | 12 | 9.05 |
| Fiber, crude | % | ND | 2.38 | 0.2 | 5.12 | 1.05 | 0.8 |
| Metabolizable | kcal/kg | 3412 | 2683 | 3188 | 2934 | 3250 | 2963 |
| Energy | | | | | | | |
| Total dietary | % | ND | ND | ND | ND | ND | ND |
| fiber | | | | | | | |
| Insoluble | % | ND | ND | ND | ND | ND | ND |
| dietary fiber | | | | | | | |
| Soluble dietary | % | ND | ND | ND | ND | ND | ND |
| fiber | | | | | | | |
| Alanine | % | 6.5208 | 7.7781 | ND | 5.1688 | 7.378 | 6.776 |
| Arginine | % | 5.4318 | 5.002 | 5.9429 | 3.2951 | 3.8378 | 3.6467 |
| Asparagine | % | 5.2272 | 5.777 | ND | 3.643 | 5.847 | 4.4845 |
| Aspartic Acid | % | 0.746 | 0.680 | 1.045 | 0.591 | 0.887 | 0.912 |
| Cystine | % | 8.488 | 8.914 | ND | 5.412 | 9.449 | 6.234 |
| Glutamic Acid | % | 6.653 | 8.278 | 6.452 | 5.219 | 7.558 | 2.710 |
| Glutamine | % | 1.630 | 2.411 | 1.642 | 1.183 | 1.773 | 1.577 |
| Glycine | % | 2.581 | 2.776 | 2.874 | 1.576 | 3.069 | 2.242 |
| Histidine | % | 4.686 | 5.412 | 4.569 | 3.593 | 5.313 | 5.002 |
| Isoleucine | % | 5.161 | 5.412 | 4.703 | 3.345 | 5.493 | 3.942 |
| Leucine | % | 1.696 | 1.045 | 1.427 | 0.984 | 1.829 | 1.454 |
| Lysine | % | 2.917 | 3.456 | 3.122 | 1.725 | 3.249 | 2.760 |
| Methionine | % | 4.62 | 5.867 | ND | 3.593 | 5.258 | 2.661 |
| Phenylalanine | % | 2.244 | 3.046 | ND | 1.819 | 2.660 | 2.513 |
| Proline | % | 2.581 | 2.956 | 2.915 | 1.918 | 2.951 | 2.513 |
| Serine | % | 0.614 | 0.455 | 0.596 | 0.442 | 0.707 | 0.912 |
| Threonine | % | 1.901 | 2.501 | 2.057 | 1.33 | 2.065 | 2.439 |
| Tryptophan | % | 3.597 | 4.002 | 3.524 | 2.709 | 3.602 | 3.277 |
| Tyrosine | ppm | ND | 0.099 | ND | 0.153 | ND | ND |
| Valine | % | 5 | 9.2 | 4.0512 | 7.57 | 6 | 7.68 |
| Taurine | % | 0.894 | 1.5 | ND | 1.136 | 1.236 | ND |
| Calcium | % | 0.14 | 0.22 | 0.18 | 0.19 | 0.15 | 0.2 |
| Chloride | % | 2.75 | 4.56 | 2.431 | 3.8 | 2.5 | 3.86 |
| Magnesium | % | 0.76 | 0.434 | 0.9 | 0.49 | 0.71 | 0.58 |
| Phosphorus | % | 0.42 | 0.77 | 0.6 | 0.65 | 0.45 | 0.51 |
| Potassium | % | ND | ND | ND | ND | ND | 0.415 |
| Sodium | ppm | ND | ND | ND | ND | ND | ND |
| Sulfur | ppm | 29.4 | 19.8 | 45 | 8.7 | 10.6 | 25.5 |
| Aluminum | ppm | ND | 0.1614 | ND | 1.756 | ND | 1.2086 |
| Copper | ppm | 220 | 726 | 90 | 712 | 217 | 375 |
| lodine | ppm | 13.1 | 48.5 | 14 | 34.2 | 17 | 14.7 |
| Iron | ppm | ND | 0.69 | 1.7 | 0.627 | 0.67 | ND |
| Manganese | ppm | 110 | 114 | 177 | 84.9 | 113 | 132 |
| Molybdenum | ppm | ND | ND | ND | ND | ND | ND |
| Selenium | ppm | ND | ND | ND | ND | ND | ND |
| Zinc | ppm | ND | ND | ND | ND | ND | ND |
| Arsenic | ppm | ND | ND | ND | ND | ND | ND |
| Chromium | ppm | ND | ND | ND | ND | ND | ND |
| Cadmium | ppm | ND | ND | ND | ND | ND | ND |
| Cobalt | ppm | ND | ND | ND | ND | ND | ND |
| Lead | ppm | ND | ND | ND | ND | ND | ND |
| Mercury | % | ND | ND | ND | ND | ND | ND |
| Nitrate | % | ND | ND | ND | ND | ND | ND |
| Nitrite (as N) | % | ND | ND | ND | ND | ND | ND |
| Fluoride | % | 0.126 | 0.092 | 3.875 | 0.1624 | 0.288 | 0.1258 |
| | (w/w) | | | | | | |
| Hydroxyproline | % | 2.431 | 0.405 | 0.4625 | 0.4871 | 3.3108 | 1.7485 |
| | (w/w) | | | | | | |
| Sulfate | % | 2.636 | 0.345 | 0.425 | 0.433 | 0.9192 | 1.1656 |
| | (w/w) | | | | | | |
| Omega 3 Total | % | 0.110 | 0.060 | 0.125 | 0.108 | 0.292 | 0.057 |
| | (w/w) | | | | | | |
| Omega 6 Total | % | 0.062 | 0.0459 | 0.038 | 0.041 | 0.109 | 0.537 |
| | (w/w) | | | | | | |
| C18:2 Omega 6 | % | 0.008 | 0.0128 | 1.68 | 0.013 | 0.092 | 0.0226 |
| (Linoleic) | (w/w) | | | | | | |
| C18:3 Omega 3 | % | 0 | 0 | 0 | 0.0067 | 0.013 | 0 |
| (alpha- | (w/w) | | | | | | |
| Linolenic) | | | | | | | |
| C20:4 | ppm | ND | ND | ND | ND | ND | ND |
| Arachidonic (all | | | | | | | |
| isomers) | | | | | | | |
| C16:0 | ppm | ND | ND | ND | ND | 0.0449 | ND |
| Hexadecanoic | | | | | | | |
| (Palmitic) | | | | | | | |
| C16:1 | ppm | ND | ND | ND | 0.2 | ND | ND |
| Hexadecenoic | | | | | | | |
| (Palmitoleic) | | | | | | | |
| C18:0 | ppm | ND | ND | ND | ND | ND | ND |
| Octadecanoic | | | | | | | |
| (Stearic) | | | | | | | |
| C18:1 | ppm | ND | ND | ND | 5.2 | ND | ND |
| Octadecenoic | | | | | | | |
| (Oleic + isomers) | | | | | | | |
| DHA (22:6) | ppm | ND | ND | ND | 59.4 | ND | ND |
| EPA (20:5) | ppm | ND | ND | ND | 4 | ND | ND |
| Vitamin A - | ppm | ND | ND | ND | 8.9 | ND | ND |
| Beta Carotene | | | | | | | |
| Vitamin A - | ppm | ND | ND | ND | 0.14 | ND | ND |
| Retinol | | | | | | | |
| Vitamin B1 - | ppm | ND | ND | ND | 0.6 | ND | ND |
| Thiamin Base | | | | | | | |
| Vitamin B1- | ppm | ND | ND | ND | ND | ND | ND |
| Thiamine | | | | | | | |
| Hydrochloride | | | | | | | |
| Vitamin B2- | IU/ | ND | ND | 3560 | ND | ND | ND |
| Riboflavin | kg | | | | | | |
| Vitamin B3- | IU/ | ND | ND | ND | 1 | ND | ND |
| Niacin | kg | | | | | | |
| Vitamin B5- | ppm | 5951 | 1940 | ND | 982.3 | 5563 | ND |
| Pantothenic | | | | | | | |
| Acid | | | | | | | |
| Vitamin B6- | % | 5.65 | 5.208 | 7.44 | 3.52 | 5.23 | 3.67 |
| Pyridoxine | | | | | | | |
| Vitamin B7- | % | 94.35 | 94.78 | 92.56 | 96.48 | 94.77 | 96.33 |
| Biotin | | | | | | | |
| Vitamin B9- | % | 14 | 31.02 | 19.12 | 25.1 | 18 | 23.8 |
| Folic Acid | | | | | | | |
| Vitamin B12- | % | 12 | 10.68 | 12.5 | 12.3 | 12 | 9.05 |
| Cobalamin | | | | | | | |
| Vitamin D | % | ND | 2.38 | 0.2 | 5.12 | 1.05 | 0.8 |
| Vitamin E | kcal/kg | 3412 | 2683 | 3188 | 2934 | 3250 | 2963 |
| Choline | % | ND | ND | ND | ND | ND | ND |

*N = 1ⁱ Donadelli et al, Poultry Science 98:1371-1378, 2019. ⁱⁱNutrient Requirements of Dogs and Cats, Animal Nutrition Series, National Academy of Sciences, 2006. ⁱⁱⁱBSM Partners ingredient data.

### Example 13

This example discusses exemplary design of genetic constructs.

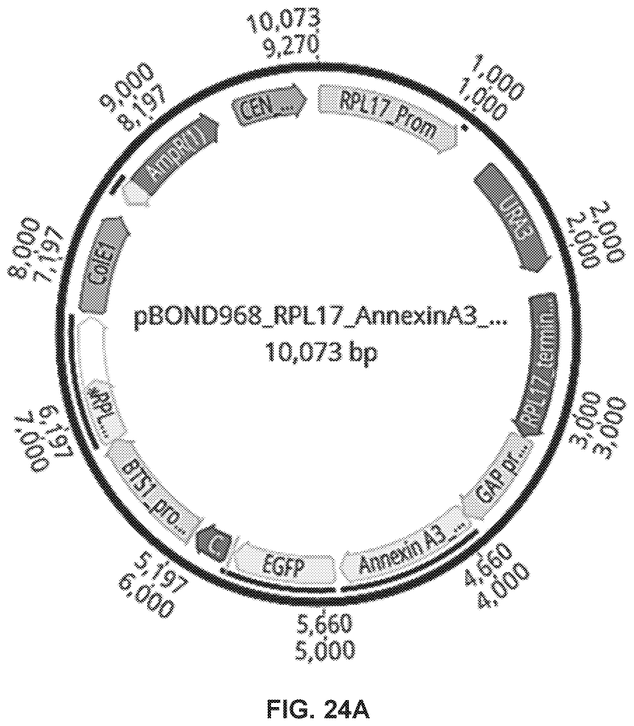

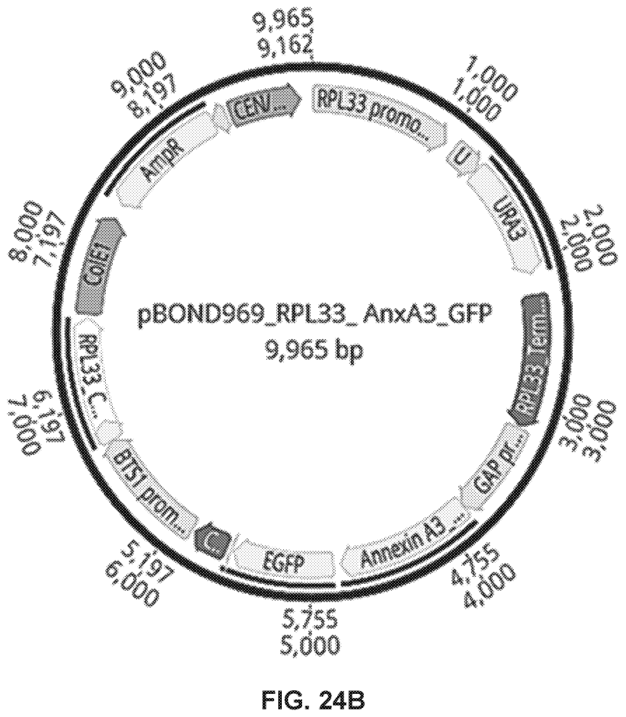

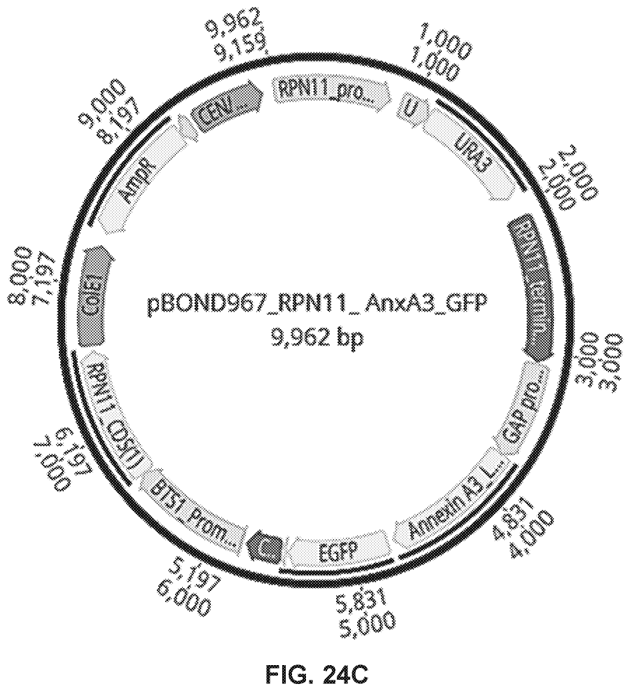

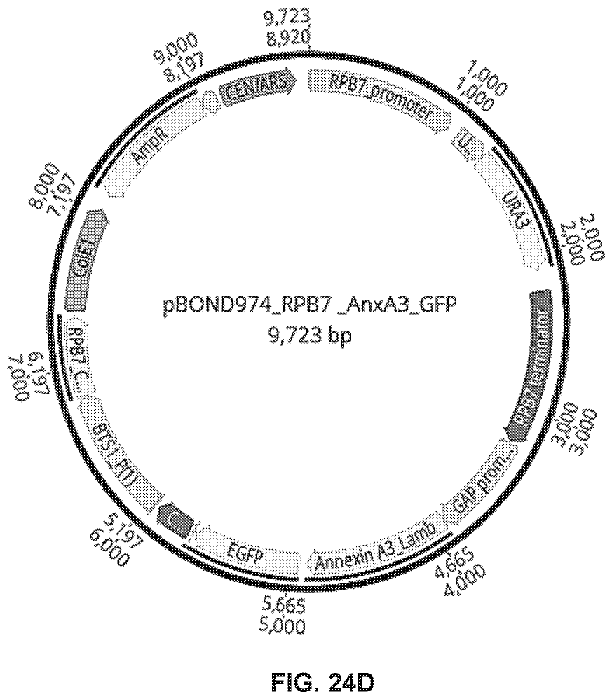

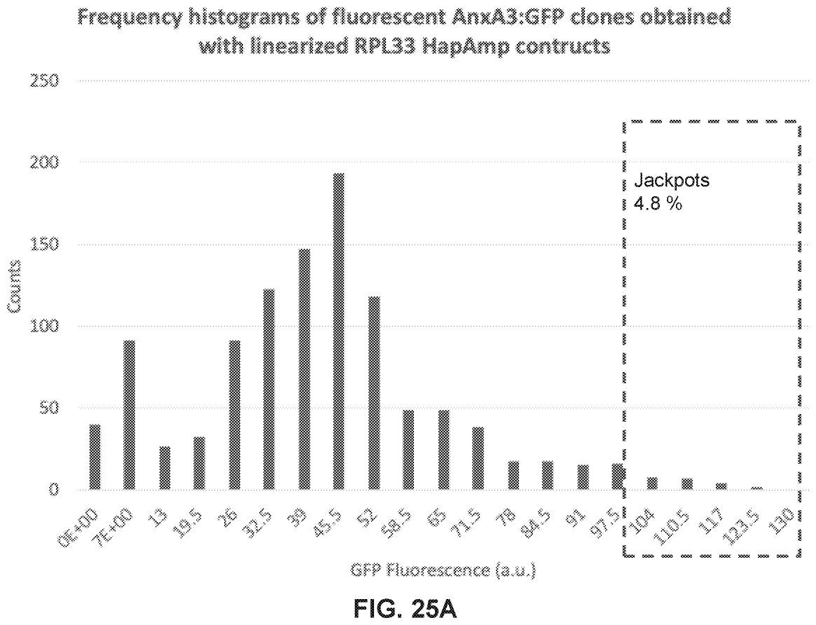

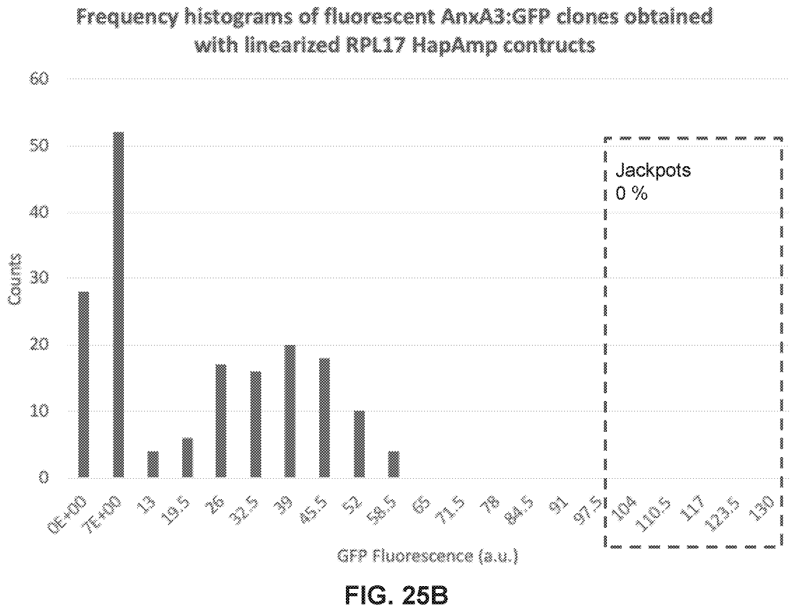

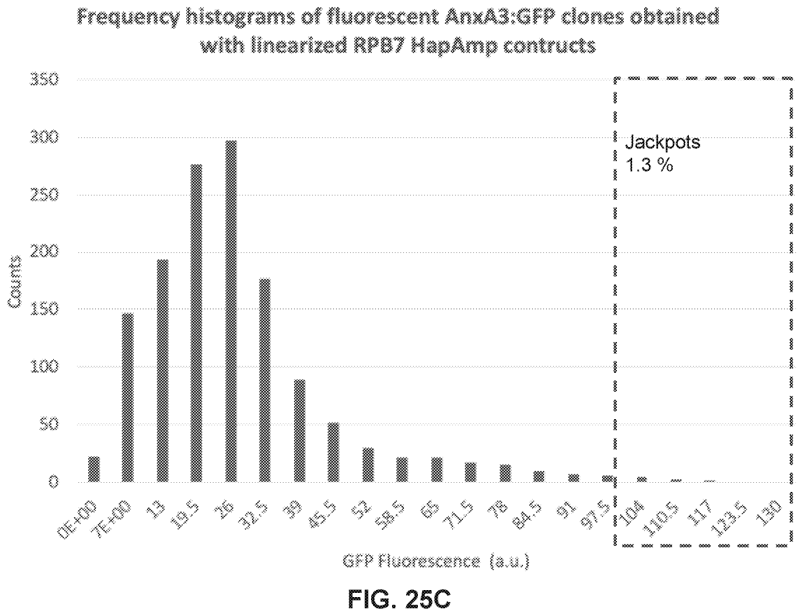

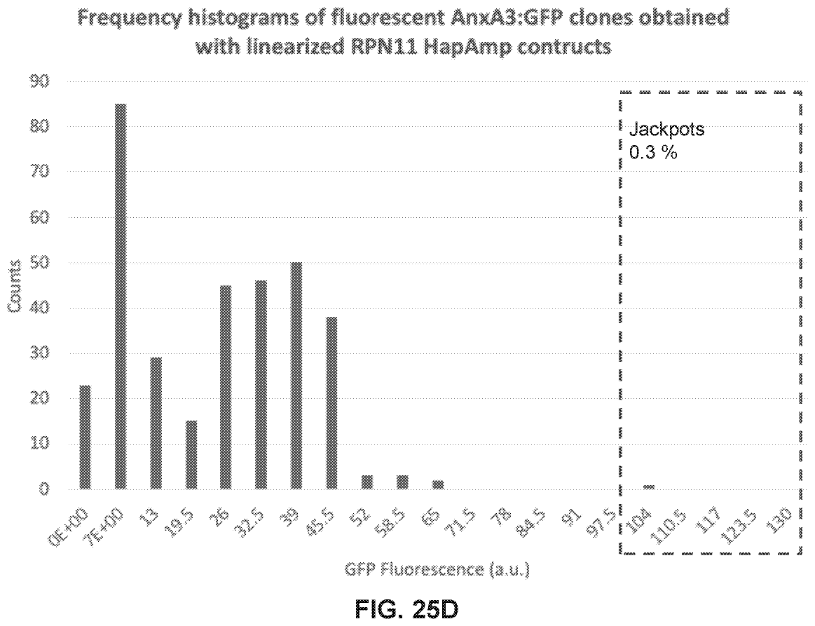

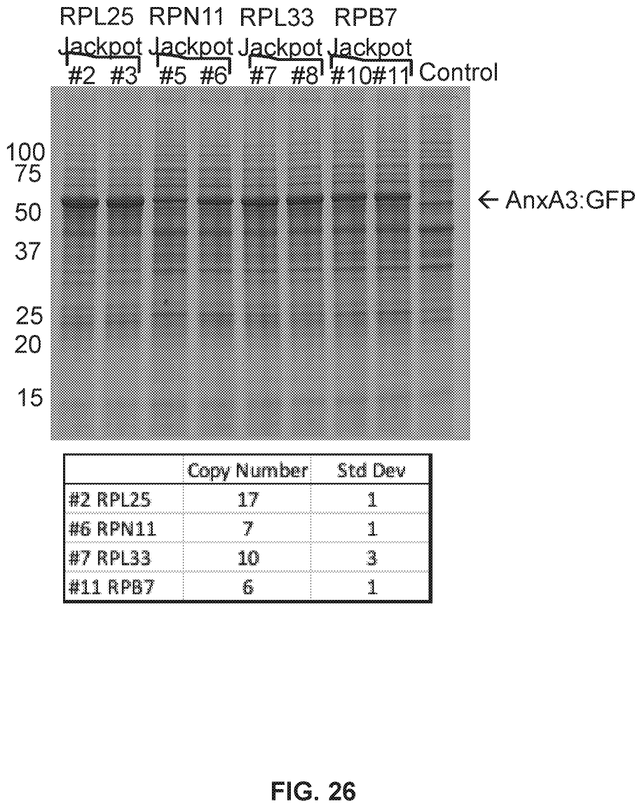

As demonstrated in FIGS. 24A-24D, each structure has recombination arms at each end. Arm 1 is homologous to the promoter region (element #1), and Arm 2 is homologous to the gene's open reading frame (element #2) of selected RPL17, RPL33, RPN11, and RPB7 loci (plasmids shown in FIG. 24A, FIG. 24B, FIG. 24C, and FIG. 24D, respectively). This allows insertion of the construct into the genome by homologous recombination. Downstream of Arm 1 is an auxotrophic selectable marker (URA3 gene) and homologous Arm 3, which is homologous to the terminator region of the specific gene (element #3). Between Arm 3 and Arm 2, is the BTS1 promoter. The BTS1 promoter is weaker than the native promoter of the targeted gene and positioned such that integration results in substitution of the native promoter of that gene with the weaker promoter.

To express heterologously the GFP protein, the expression cassette containing the coding sequence for GFP driven by the yeast TDH3 promoter and Cycl terminator is inserted between Arm 3 and the BTS1 promoter. Driving expression through a weaker promoter attenuates the protein yield from each copy of the specific haploinsufficient gene. This, in turn, is expected to decrease the growth rate of S. cerevisiae. Native gene amplification of the region between homologous Arm 3 and the native terminator (element #3) will then occur as this yeast evolves towards faster growth.

### Example 14

This example discusses FIGS. 1A, 1B, 27, 28.

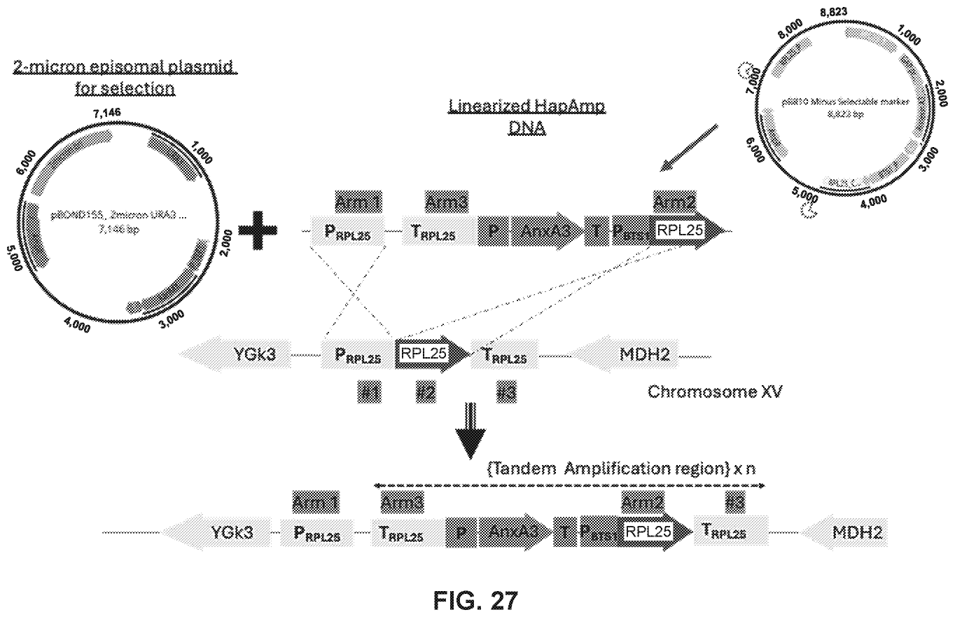

The genetic structure for chromosomal integration and gene amplification at RPL25 in yeast, as modified from Peng et al. for the HapAmp expression of Annexin A3 (AnxA3) (FIGS. 1A and 1B). This structure includes recombination arms at each end. Arm 1 is homologous to the promoter region of a haploinsufficient gene (element #1), while Arm 2 is homologous to the open reading frame of the haploinsufficient gene (element #2). This allows for insertion of the construct into the genome via homologous recombination. Downstream of Arm 1 is an auxotrophic selectable marker (URA3) (FIG. 1A) for transformation selection and homologous Arm 3, which corresponds to the terminator region of the haploinsufficient gene (element #3). The selectable marker can be recycled via Cre-loxP sites system. FIG. 1B depicts a design which does not comprise a selectable marker. FIGS. 27 & 28 demonstrate linearized HapAmp DNA in a plasmid with, and a plasmid without, a selectable marker. Between Arm 3 and Arm 2 is the BTS1 promoter, which is weaker than the native promoter of the haploinsufficient gene. The promoter is positioned such that integration results in the substitution of the native promoter with the weaker BTS1 promoter. To heterologously express AnxA3, the expression cassette containing AnxA3, the yeast TDH3 promoter, and the CYC1 terminator are inserted between Arm 3 and the weaker BTS1 promoter. Driving expression through a weaker promoter attenuates the protein yield from each copy of the RPL25 haploinsufficient gene. This is expected to decrease the growth rate in yeast. Native gene amplification of the region between Arm 3 and the native terminator (element #3) will occur as yeast evolves towards faster growth.

In this example, we describe a method of generating a food composition where we provide at least one genetic construct comprising: a first promoter homologous to at least a portion of the native promoter region of a haploinsufficient gene tied to yeast fitness, a RPL25 promoter in this case; a terminator homologous to at least a portion of the native terminator of the haploinsufficient gene tied to yeast fitness, a RPL25 terminator in this example. The genetic construct also includes one or more genes encoding heterologous polypeptide(s) of interest, we used AnxA3 in this example; and a synthetic open reading frame (ORF) homologous to at least a portion of a native ORF of the haploinsufficient gene tied to yeast fitness, which was a RPL25 ORF in this example.

Next, we integrated the at least one genetic construct into the haploinsufficient gene tied to yeast fitness to produce one or more transformed yeast strains, wherein the integrating generates a tandem amplification region.

Next, we screened the transformed yeast strains based on (i) co-transformation with 2-micron episomal plasmid conferring antibiotic and/or auxotrophic selection and/or (ii) heterologous polypeptide of interest content and a gene copy number of the coding sequence of the heterologous polypeptide of interest.

### Example 15

This example discusses an exemplary model for amino acid composition prediction.

To predict the potential amino acid profile of a strain consisting of multiple proteins, a few crucial measurements are required to complete the mass balance: (1) Total Kjeldahl Protein, (2) Total % Protein of POIs by mass, (3) Measured Amino Acid Profile of the Background Strain, (4) Measured Amino Acid Profile of the POI Producing Strain.

Information on the amino acid sequence of the POIs and their corresponding molecular weights will also be required.

Definitions for equation E3 (E3) below:
Mass: Total dry weight of sample
% TPk: Total Kjeldahl Protein=TP/Mass
TP*: Total Proteome Protein (crude protein)
% POI: POI as measured by Mass Spectrometry (POI/TP*)
Mass Fraction: (Amino frequency×Amino acid M.W.)/Protein M.W.
A.A.: Amino acid

The principle of calculation is based on the following equation (E3):

$$( \% TPk ) \times ( \% POI ) \times ( \% A . A . ) i = ( A . A . ) i , for all 20 amino acids , \qquad ( E3 )$$

$$where ( TPk / Mass ) \times ( POI / {TP}^{*} ) \times ( A . A . / POI ) i = ( A . A . / Mass ) i ; Assumptions : {TP}^{*} = \% TPk$$

Or, the % mass contribution of the ith amino acid of any protein is equal to the mass fraction of the individual amino acid, multiplied by the mass fraction of the POI on dry mass basis.

As an example: A protein expressed at 20%, with 5% cysteine by mass, in a cell mass that has 60% Total Kjeldahl Protein, will have 0.6% Cysteine on a dry mass basis, as shown:

$$100 g * 0.6 = 60 g protein , 60 g * 0.2 = 12 g POI , 12 g * 0.05 = 0.6 g Cysteine$$

To determine the contribution from multiple proteins one need only perform the same calculation, then sum all the ith amino acids, to determine the cumulative contribution to the final mass.

Based on this mass balance principle, we can predictively model the amino acid profile of a future strain or determine the shift in the background proteome caused by over expression of our POI. As above, the mass fractions of the individual amino acids are calculated then summed. This array of amino acid mass fractions now each represent a 15% percent contribution to amino acid profile of our final cell mass. Next, we produced a similar amino acid array from the Measured Amino Acid Profile, to which we assign a (100-15) % contribution. Finally, the weighted column containing the predicted POI contribution to mass is added to the weighted column for the strain background.

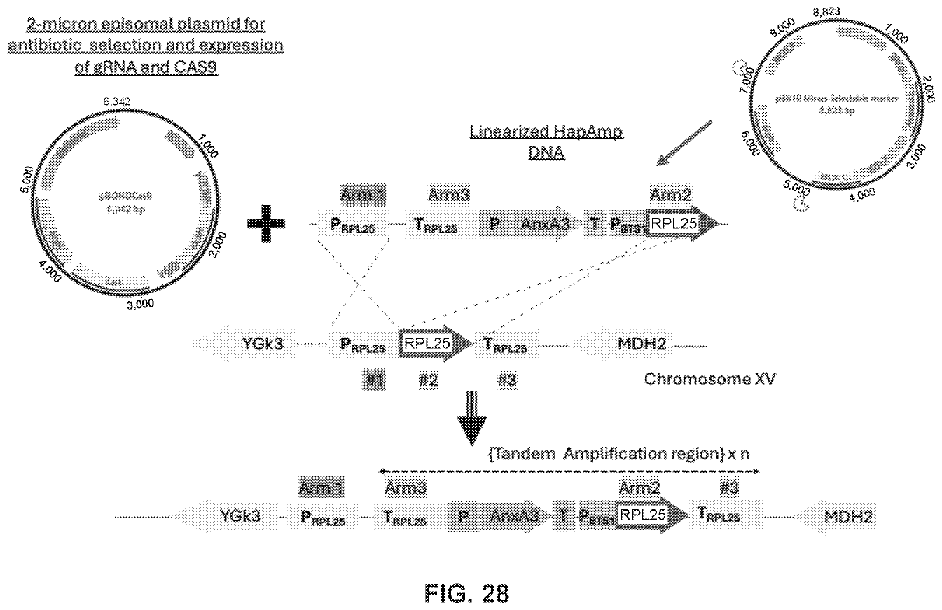

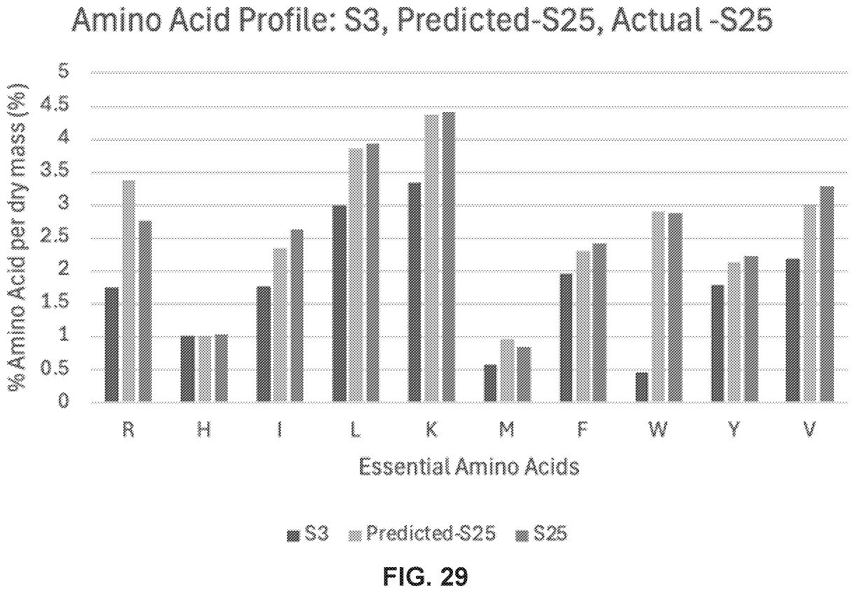

FIG. 29: The left most column for each of the amino acids pertains to the measured concentration for S3, the wild-type organism, the center column delineates the model predicted amino acid profile for our modified organism, S25. The third column pertains to the measured amino acid concentration for S25. (D+N and E+Q columns, are included in the model prediction, since the assay used for quantification does not discern aspartate/asparagine nor glutamate/glutamine.)

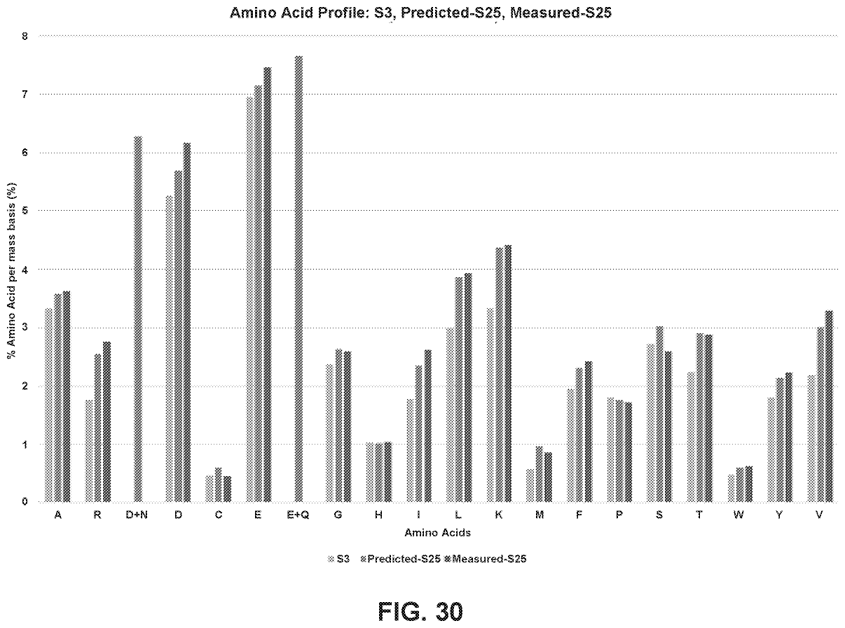

FIG. 30: The left most column for each of the amino acids pertains to the measured concentration for S3, the wild-type organism, the center column delineates the model predicted amino acid profile for our modified organism, S25. The third column pertains to the measured amino acid concentration for S25. (D+N and E+Q columns, are included in the model prediction, since the assay used for quantification does not discern aspartate/asparagine nor glutamate/glutamine.)

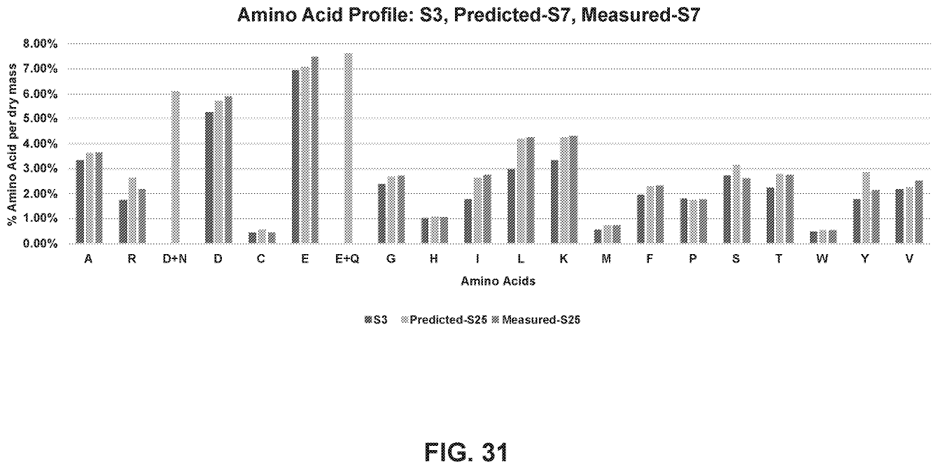

FIG. 31: The left most column for each of the amino acids pertains to the measured concentration for S3, the wild-type organism, the center column delineates the model predicted amino acid profile for our modified organism, S7. The third column pertains to the measured amino acid concentration for S7. (D+N and E+Q columns, are included in the model prediction, since the assay used for quantification does not discern aspartate/asparagine nor glutamate/glutamine.)

### Example 16

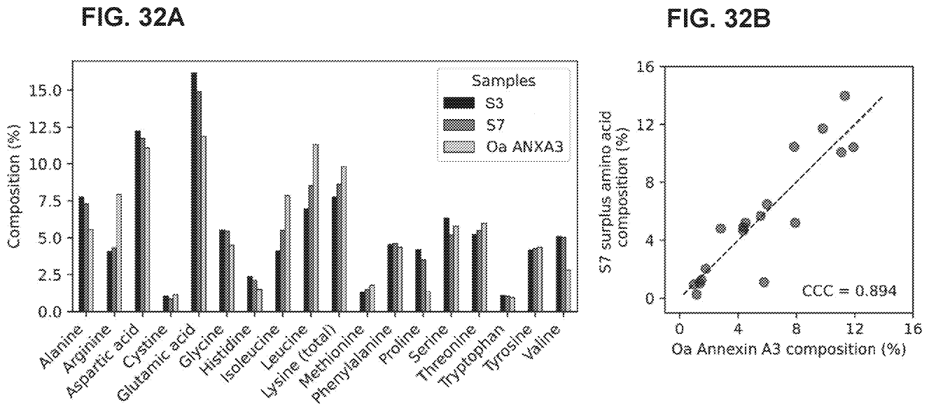

The amino acid composition of total cellular protein was analyzed for both wild-type and strain S7 and compared to that of pure Annexin A3 from Ovis aries (FIG. 32, left). In most cases, the amino acid composition of strain S7 falls between that of the wild-type and pure Annexin A3, suggesting that the high expression of Annexin A3 influences the amino acid content of strain S7. To quantify this, we calculated the surplus amino acid content of strain S7 by first assuming protein content is increased by 28.2% due to heterologous protein expression. This increase is based on post-fermentation mass spectrometry samples (n=5) in which Annexin A3 is measured to be 22% of total protein, i.e. 0.22/(1-0.22) 0.282. We then subtracted wild-type amino acid content from that of strain S7. The resulting surplus amino acid composition is highly concordant (0.894) with the composition of pure Annexin A3 (FIG. 32, right). This indicates that a substantial portion of the difference in amino acid composition between wild-type and strain S7 can be attributed to the heterologously expressed Annexin A3.

### Example 17

This example discusses Gallus gallus Annexin A4 construct: Copy number and stability

Copy number. Copy number of Gallus gallus Annexin A4 (GgAnnexinA4) integrated into the RPL25 site was estimated by DNA sequencing.

Biological replicates from multiple fermentations as well as technical preparations from single tank fermentations show an estimated 32.3+/−4.8 copies of the integration construct in post fermentation samples. DNA sequencing limitations did not allow for full spanning reads of the integration locus (estimated to be >140,000 bp)—but analysis of read depth of the genomic region of interest gives reproducible estimates of copy number based on the number of sequencing calls of the region compared to unengineered strain.

Copy number change over time. To examine a potential change in tandem repeat copy number over time, due to natural tandem repeat amplification over time, cells were propagated in shake flasks for 100 generations (i.e. doublings) and sampled for whole genome sequencing and read depth at the locus of interest. Time 0 is considered culture inoculation and enough biomass for whole genome sequencing was obtained after ~6.5 doublings with an estimated copy number via read depth of 42.5 copies. After ~45 doublings (7 days) the estimated copy number via read depth is 50.3. After ~101 doublings, read depth approximates 44.8 insert copies per cell.

Production stability. The change in insertion copy number over time is an anticipated and expected outcome based on the naturally occurring phenomenon of tandem repeat amplification/reduction due to the introduction of the fitness defect. The dynamic nature of the culture population will, through natural selection, favor strains that produce sufficient amounts of the limiting protein (in this case RPL25) for maximum growth and fitness.

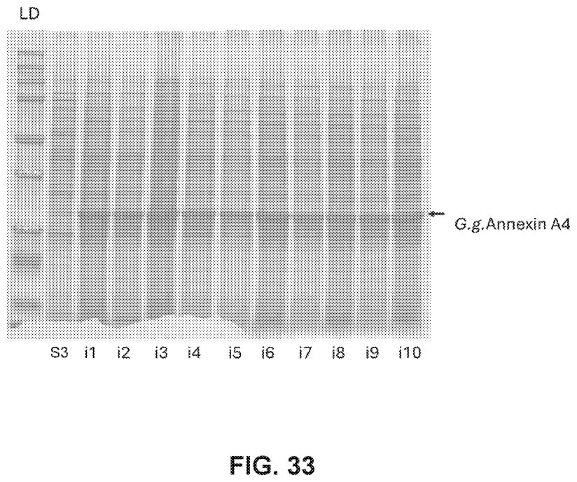

To demonstrate that the protein of interest is stable and present in post fermentation samples (~40 doublings), cells were plated for isolated colonies. 38 single colonies were then grown for 24 hours in production media, and total protein was isolated from samples and examined by SDS-PAGE with Coomassie staining for the presence of GgAnnexinA4. In all isolates tested, the Coomassie stained band representing GgAnnexinA4 is present—confirming the strain is stable in regard to production of the protein of interest (FIG. 33).

### Example 18

This example discusses CGQ and Growth Rate Comparisons of Background v Engineered Yeast.

Growth rates are measured using Cell Growth Quantifiers and DOTS software from Scientific Bioprocessing Inc. This equipment allows for continuous measurement of flask growth profiles by using reflected light as a proxy for flask turbidity and cell density. The growth rate beyond the diauxic shift does not differ significantly between strains, and growth on glucose provides the best indicator for relative robustness between isolates. The growth rate μ, in the unit h⁻¹, is calculated from the equation [(ln(D_{f}/Dᵢ))/(T_{f}−Tᵢ)], where D_{f} and D₁ stand for the final and initial culture densities, on an interval spanning T_{f}, final time, and Tᵢ, the initial time. A one-hour time interval is generally used, as this best approximates the average slope on the given interval of the growth phase. In other words, the measured growth rate on glucose, on a 1-hour interval, is the average growth rate on glucose, on the entire interval (from start to diauxic shift).

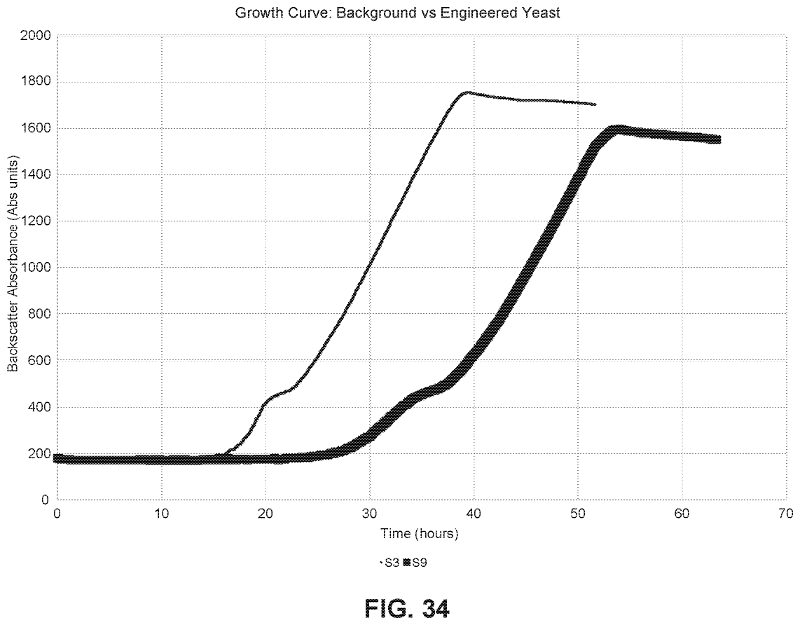

As an example, the engineered strain S9 represents the lower bound for an acceptable growth rate. The strains were grown for 10 generations to determine the increase fermentation run time as a function of growth rate, μ (FIG. 34). From this data we ascertain that for every 10 generations of cell expansion, 16 hours will be added to the expected fermentation run time, when compared to the WT control, S3 (FIG. 34). Since scale-up fermentations are expected to require 30-40 generations of expansion, this growth rate difference would add 48-64 hours to the entire production campaign. As previously described, the growth rate on ethanol beyond the diauxic shift does not vary significantly and the deviation in growth, between strains, can be determined by calculating the growth rate on glucose.

### Example 19

This example discusses the amino acid pool hypothesis for protein stacking as well as the protein burden hypothesis and haploid insufficiency.

Amino Acid Pool Hypothesis for Protein Stacking. For any given organism there exists a homeostatic amino acid pool, the relative proportions of which can vary based on genetics, metabolism, cell cycle, and other environmental conditions such as feedstock and nutritional supplementation. Therefore, a decrease in the demand on the amino acid pool should improve performance in strains. Excessive demand on the amino acid pool caused by overproduction of our exogenous protein may limit resources available to produce the native proteins and enzymes required for robust growth in yeast strains. When the addition of alternate proteins at secondary expression sites reduces expression of protein #1, the demand on the amino acid pool for the specific amino acids, from protein #1, may also be reduced, allowing for faster and more robust growth in the modified strains. Additionally, when the excessive draw from the amino acid pool pertains to a specific amino acid or an amino acid which is not produced naturally in significant quantities, availability of these amino acids may become a bottleneck to the production of protein #1. The expression of an alternate protein with a complementary amino acid profile would not have this limitation and would allow for greater expression of total exogenous protein.

Protein Burden Hypothesis and Haploid Insufficiency. The burden to growth exhibited by our selected exogenous proteins, vary depending on protein of interest and expression levels. Although the genetic make-up of the modified strains is stable and uniform, minor differences in reactor conditions can lead to variable production of exogenous protein within each individual cell. This variable expression per cell can lead to diminished growth rates and reduced cell dry weight yields in the production reactor. Based on the ToxAmp Hypothesis of haploid insufficiency, there is a balance between the copy number of the Haploid Insufficient Gene and the accompanying Exogenous Compound (protein) [2]. Our selection algorithm allows us to select from three sub populations: (1) slow growth/high yield (2) robust growth/medium to high yield, and (3) fast growth/low to medium yield. This bimodal selection method, specifically selects for robust growth under high stress conditions, allowing us to eliminate poor growing strains and decrease the number of strains we must screen. We are able to vary the burden of the expression cassette by the addition of secondary proteins of interest to the same expression cassette or to alternate expression sites. This allows us to better optimize strain robustness and overall production of total protein and exogenous protein of interest.

References corresponding to Example 19, each of which are incorporated herein by reference in its entirety for all purposes: Watson, Amino-acid Pool Composition of Saccharomyces cerevisiae as a Function of Growth Rate and Amino-acid Nitrogen Source, Micro. Soc. 96:2 (1976); Evans et al., The integration of tandem gene repeats via a bacterial type-II toxin-antitoxin-mediated gene amplification (ToxAmp) system and stability visualisation in Saccharomyces cerevisiae, BioRxiv, (2024).

### Example 20

This example discusses the utility of stacking multiple proteins in Bond Strains.

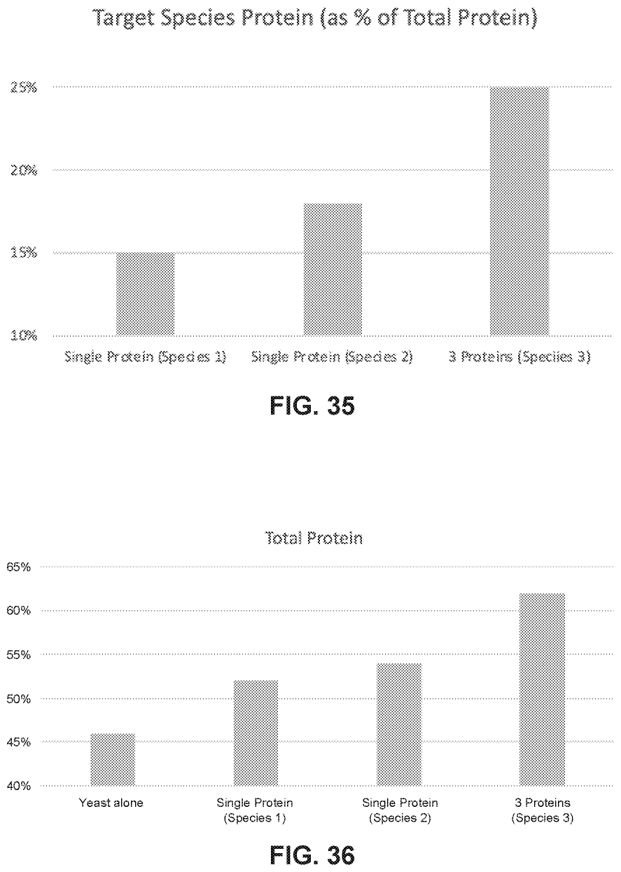

The choice of protein expressed in Bond production strains is based on compatibility with robust S. cerevisiae growth and high-level expression of the target species protein. Although a single protein can achieve high levels of expression in Bond's strains, the ability to stack multiple proteins in the same S. cerevisiae background results in both a higher level of total protein and a higher % of target species present in Bond's final product (see results depicted in FIG. 35 and FIG. 36).

One or a plurality of recombinant proteins can be produced in one organism, or one strain, thereby allowing the amino acid profile to be tailored to the particular nutritional needs of targeted companion and other animals, including humans. Alternatively, a single recombinant animal protein can be produced in one strain (or organism) and mixed with a protein or proteins produced in a different strain (or organism) to yield a final product with the desired proportions of amino acids and other nutrients. Thus, the amino acid profile (and/or the profile of other nutrients) can be customized for the targeted animal, including pets and humans. Over-expressing one or several animal proteins in an organism can increase the content of specific amino acids that are prevalent in the animal proteins, making it possible to customize the amino acid profile.

### Example 21

This example discusses the copy number and stability of the Ovies aries Annexin construct.

Copy number. Copy number of Ovies aries Annexin A3 (OaAnnexinA3) integrated into the RPL25 site was estimated by DNA sequencing.

Biological replicates from multiple fermentations as well as technical preparations from single tank fermentations show an estimated 31.5+/−0.9 copies of the integration construct in post fermentation samples. DNA sequencing limitations did not allow for full spanning reads of the integration locus (estimated to be >140,000 bp)—but analysis of read depth of the genomic region of interest gives reproducible estimates of copy number based on the number of sequencing calls of the region compared to unengineered strain.

Copy number change over time. To examine a potential change in tandem repeat copy number over time, due to natural tandem repeat amplification over time, cells were propagated in shake flasks for 100 generations (i.e. doublings) and sampled for whole genome sequencing and read depth at the locus of interest. Time 0 is considered culture inoculation and enough biomass for whole genome sequencing was obtained after ~6.5 doublings with an estimated copy number via read depth of 32.3 copies. After ~45 doublings (7 days) the estimated copy number via read depth is 34.1. After ~101 doublings, read depth approximates 37.5 insert copies per cell.

Production stability. The change in insertion copy number over time is an anticipated and expected outcome based on the naturally occurring phenomenon of tandem repeat amplification due to the introduction of the fitness defect. The dynamic nature of the culture population will, through natural selection, favor strains that produce sufficient amounts of the limiting protein (in this case RPL25) for maximum growth and fitness.

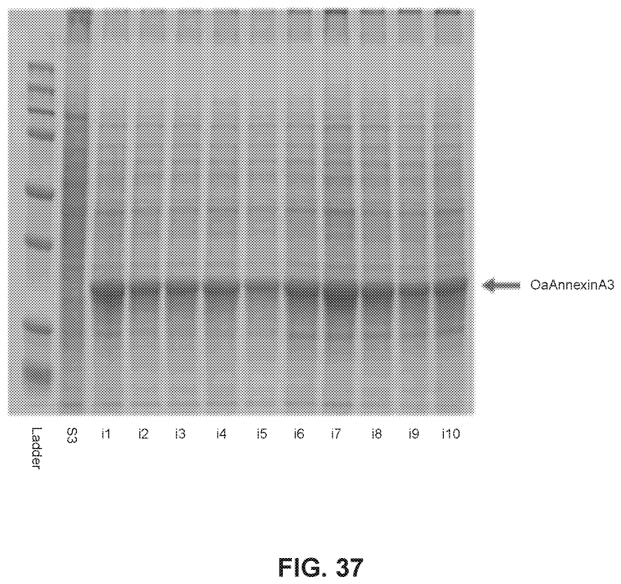

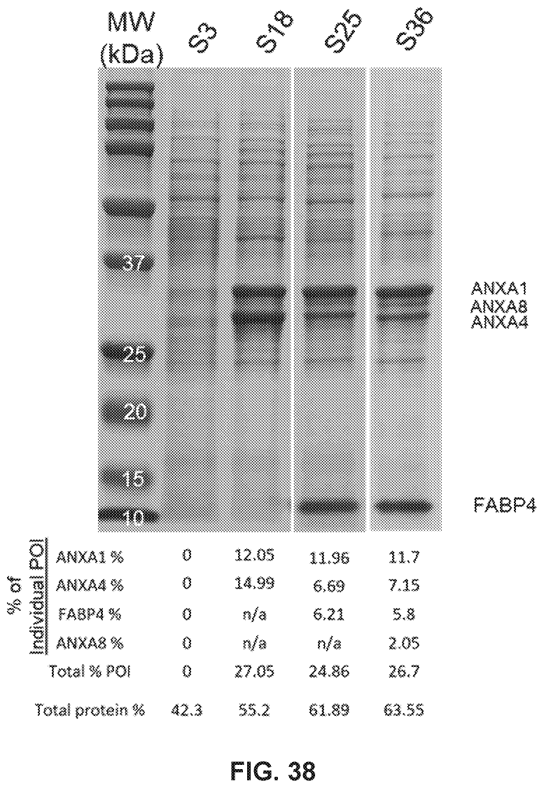

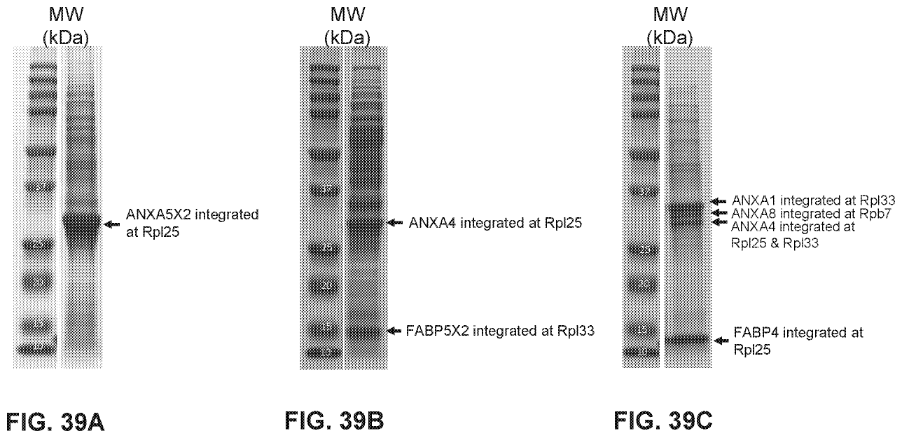

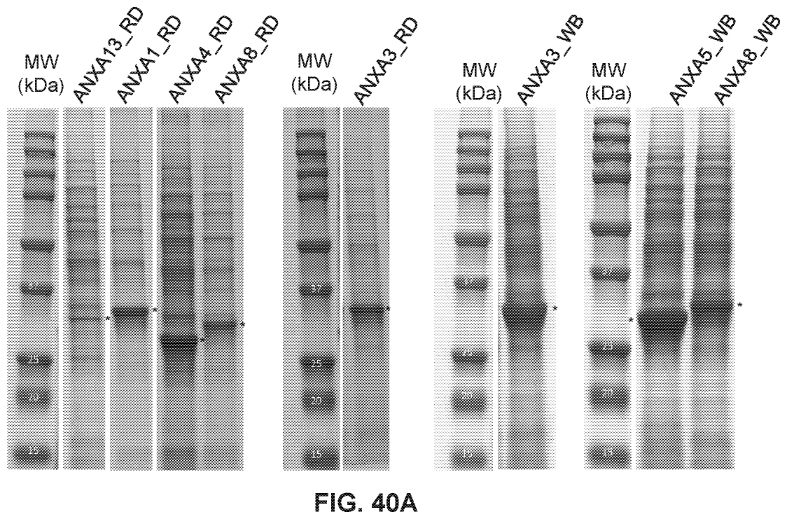

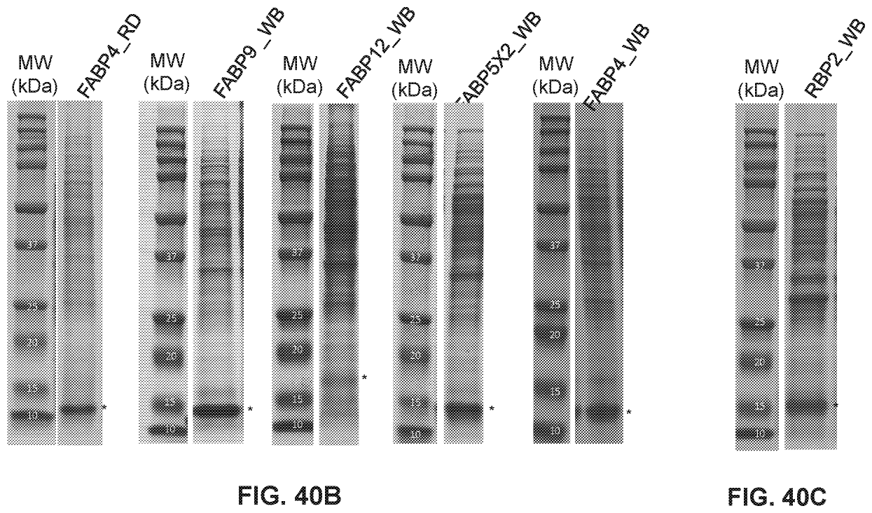

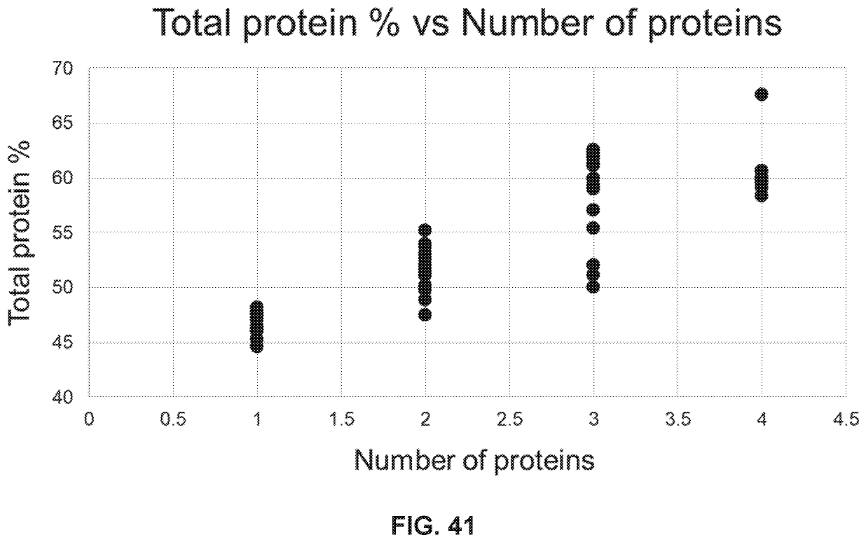

To demonstrate that the protein of interest is stable and present in post fermentation samples (~40 doublings), cells were plated for isolated colonies. 53 single colonies were then grown for 24 hours in production media, and total protein was isolated from samples and examined by SDS PAGE with Coomassie staining for the presence of OaAnnexin A3. In all isolates tested, the Coomassie stained band representing OaAnnexin A3 is present—confirming the strain is stable in regard to production of the protein of interest (FIG. 37).

### Example 22

This example discusses exemplary and preferred amino acid compositions for food compositions for pet food or feed. The methods and compositions described herein may be tuned in order to achieve the following amino acid profiles.

Optimal amino acid composition. Whole dried egg is the “gold standard” for amino acids. Whole dried egg contains (on a dry basis): 2.94% arginine; 1.16% histidine; 2.67% isoleucine; 4.19% leucine; 3.52% lysine; 1.53% methionine; 2.61% phenylalanine; 2.35% threonine; 0.6% tryptophan; 2.99% valine

Essential amino acids (for dogs). Ranges based on dry matter: Arginine: 2.9% optimal, 2.1-4.6% min/max, 2.5-3.8% preferred; Histidine: 1.2% optimal, 0.8-1.9% min/max, 1-1.6% preferred; Isoleucine: 2.7% optimal, 1.7-4.3% min/max, 2.2-3.5% preferred; Leucine: 4.2% optimal, 2.9-6.7% min/max, 3.5-5.5% preferred; Lysine: 3.5% optimal, 2.4-5.6% min/max, 3-4.6% preferred; Methionine: 1.5% optimal, 0.7-2.4% min/max, 1.3-2% preferred; Phenylalanine: 2.6% optimal, 1.4-4.2% min/max, 2.2-3.4% preferred; Threonine: 2.35% optimal, 1.6-3.8% min/max, 2-3.1% preferred; Tryptophan: 0.6% optimal, 0.4-1% min/max, 0.5-0.8% preferred; Valine: 3% optimal, 2-4.8% min/max, 2.6-3.9% preferred

Reference cited in this Example 22, which is hereby incorporated by reference in its entirety: Nutrient Requirements of Dogs and Cats, Animal Nutrition Series, National Academy of Sciences, 2006.

### Example 23

This example discusses our hypotheses and findings as to why we expect certain proteins, specifically animal-derived annexins, FABPs, and retinoid-binding proteins, to achieve enhanced expression levels using the modified HapAmp method as well as result in food ingredient compositions, and other compositions with high protein content.

### Example 23A: Animal-Derived Annexins, Fatty Acid-Binding Proteins (FABPs), and Retinoid-Binding Proteins (RBPs): What do they have in Common and why do they Express so Well in Yeast?

Binding of Hydrophobic Molecules. The fact that these animal-derived proteins (annexins, fatty acid-binding proteins, and retinoid-binding proteins), which belong to a larger family, intracellular lipid-binding proteins, are expressed well in yeast may have a connection to their ability to bind hydrophobic molecules. Proteins that bind hydrophobic molecules often have a well-defined hydrophobic core, which contributes to their stable structure. This intrinsic stability might make them easier to fold correctly when expressed in a foreign host like yeast. Proper folding is crucial for functional expression, and these proteins might inherently possess properties that allow them to fold correctly in the yeast cellular environment.

Annexins bind to phospholipids, which are hydrophobic molecules found in cellular membranes. This binding is calcium-dependent. FABPs bind fatty acids and other hydrophobic lipid molecules within the cytoplasm, aiding in their transport and metabolism. Retinol-binding proteins (RBPs) bind retinol (vitamin A alcohol), a hydrophobic molecule, and transport it in the bloodstream to target tissues.

Yeast cells, like animal cells, have lipid-rich membranes and produce various hydrophobic molecules. Proteins that naturally interact with hydrophobic molecules may be more compatible with the yeast cellular environment, allowing them to integrate well into yeast's lipid milieu. This compatibility could enhance their expression and functionality in yeast.

Yeast has a well-established system for processing and secreting proteins, especially those with hydrophobic characteristics. The endoplasmic reticulum (ER) and Golgi apparatus in yeast are involved in the proper folding, post-translational modifications, and trafficking of proteins. Proteins that bind hydrophobic molecules might leverage these pathways effectively, ensuring correct processing and functional expression.

The hydrophobic binding sites in these proteins may also protect them from aggregation or degradation within the yeast cell. Proteins that interact with hydrophobic ligands are often designed to shield these hydrophobic regions, which could reduce the likelihood of misfolding or proteolytic degradation, common issues in recombinant protein expression.

The ability to bind hydrophobic molecules might provide these proteins with a structural robustness and compatibility with yeast's lipid-rich environment, facilitating their successful recombinant expression. This shared characteristic could help them maintain their functional conformation and interact appropriately with yeast cellular machinery, leading to higher expression levels and activity.

Specific roles. Annexins, although primarily membrane-associated, can participate in vesicle trafficking and the movement of membranes within cells. FABPs facilitate the intracellular transport of fatty acids and lipids, delivering them to specific organelles or sites within the cell for metabolism or signaling. RBPs transport retinol in the bloodstream, delivering it to various tissues where it can be utilized or stored.

Annexins are involved in processes like membrane repair, signaling, and calcium regulation, all of which are critical for cellular homeostasis. FABPs play a role in regulating lipid metabolism and signaling pathways, influencing energy homeostasis and cellular responses to metabolic changes. RBPs regulate the availability of retinol, influencing processes such as vision, immune function, and cellular differentiation.

Annexins participate in signal transduction pathways related to membrane dynamics and calcium signaling. FABPs are involved in lipid signaling pathways, influencing processes like inflammation and metabolic regulation. RBPs indirectly influence signaling pathways by regulating retinol availability, which can affect gene expression and cellular differentiation.

Annexins are found at the cytosolic face of cellular membranes, particularly in response to calcium signaling. FABPs are located in the cytoplasm, where they interact with intracellular membranes and organelles. RBPs circulate in the bloodstream and interact with cell surface receptors to deliver retinol. These proteins, though distinct in their specific roles and molecular targets, share common themes such as binding hydrophobic molecules, facilitating transport, and participating in the regulation of key cellular processes.

### Example 23B: Animal-Derived Annexins, Fatty Acid-Binding Proteins (FABPs), and Retinoid-Binding Proteins (RBPs): Why are these Expressed so Well in Yeast?

Low similarity to native yeast proteins. The low similarity of these animal-derived intracellular lipid binding proteins (such as annexins, fatty acid-binding proteins, and retinoid-binding proteins) to native yeast proteins could contribute to their high expression levels when produced recombinantly in yeast. These proteins, because they have low similarity with yeast's native proteins, are thought to be less likely interact with native cell proteins/processes and therefore be ‘inert’.

Yeast cells have proteases that degrade misfolded or unneeded proteins. Proteins that are highly similar to native yeast proteins might be recognized and targeted by these proteases for degradation, especially if they are perceived as misfolded or non-functional. In contrast, proteins with low similarity to native yeast proteins may not be as easily recognized by these proteases, reducing the likelihood of degradation and leading to higher expression levels.

Proteins that differ significantly from native yeast proteins might face less competition for binding sites on cellular machinery, such as chaperones or transport systems. Native yeast proteins might compete for the same resources, potentially lowering the efficiency of expression for proteins that closely resemble yeast endogenous proteins. Unique, foreign proteins might therefore be processed more efficiently, resulting in higher expression levels.

Proteins with sequences that are distinct from native yeast proteins might fold differently, avoiding interactions that could lead to misfolding or aggregation. Misfolded proteins often trigger cellular stress responses, such as the unfolded protein response (UPR), which can reduce overall protein expression. Proteins with low similarity to native yeast proteins might fold more independently, avoiding such issues and thus achieving higher expression levels.

Yeast cells can sometimes recognize foreign proteins as potential threats, leading to stress responses that reduce protein expression. Proteins that closely resemble yeast's own proteins might inadvertently trigger such responses if they're mistaken for aberrant forms of native proteins. In contrast, proteins that are sufficiently different might avoid triggering these responses, allowing for more efficient expression.

The low similarity of these animal-derived proteins to native yeast proteins likely contributes to their high expression levels by reducing proteolytic degradation, minimizing competition for cellular resources, preventing misfolding and aggregation, and avoiding cellular stress responses. These factors together create a more favorable environment for the efficient production of these recombinant proteins in yeast.

Size. The size of these proteins (in aspects, ranging from 10 to 45 kDa) could play a significant role in their high expression levels in yeast. Proteins within this size range are often more likely to fold correctly and efficiently within the yeast cell's endoplasmic reticulum (ER) and Golgi apparatus. Proteins that are too large may face difficulties with correct folding or post-translational modifications, leading to misfolding or aggregation. Smaller proteins are generally less demanding on the cell's protein synthesis and folding machinery compared to larger proteins. They are less likely to overwhelm the cell's resources or trigger stress responses, allowing for higher expression levels. Proteins in this size range are often efficiently transported through cellular compartments and secretory pathways. They are less likely to accumulate in the ER or other cellular compartments, reducing the likelihood of inducing stress responses or bottlenecks in the secretion process. Smaller proteins are less likely to form aggregates compared to very large proteins. Aggregates can be toxic to cells and lead to reduced expression levels. The smaller size may help maintain solubility and reduce the risk of aggregation. Many yeast expression systems are designed to handle proteins within this size range effectively. The protein size is well-suited to the capabilities of the yeast cell's translation and processing machinery. Proteins that are too large or complex can induce stress responses in yeast cells, such as the unfolded protein response (UPR). Smaller proteins are less likely to induce such responses, contributing to higher expression levels.

The size of the proteins (e.g., 10-45 kDa) likely contributes to their high expression levels in yeast by promoting efficient folding, reducing the load on cellular machinery, facilitating transport and secretion, and minimizing the risk of aggregation and cellular stress. Proteins within this size range are well-suited to the yeast cellular environment, enhancing their overall expression and stability.

Stability. Several factors that relate to the structure and function can contribute to the stability of these proteins. Annexins generally have stable structures due to their well-defined calcium-binding sites and their role in membrane interactions. Their stability might contribute to a relatively long half-life in the cell. Annexins do not typically have strong degradation signals, which can help them persist longer. However, their stability can be influenced by their interactions with membranes and the presence of calcium. Their interaction with membranes may protect them from degradation or influence their stability, although this can also depend on the cellular context and membrane dynamics.

Fatty Acid-Binding Proteins (FABPs) are designed to bind and transport fatty acids, and their hydrophobic binding sites contribute to a stable, functional structure. FABPs generally do not have specific degradation signals, which might contribute to their longevity within the cell. The binding of hydrophobic molecules can stabilize FABPs and protect them from proteolytic degradation. The interaction with fatty acids might also influence their cellular stability.

Retinoid-binding proteins are stable due to their specific binding to various retinoids, including retinol, retinal, and retinoic acid. Their structure is optimized to protect these hydrophobic molecules and facilitate their transport and regulation. The binding of retinoids may help stabilize the protein, reducing its turnover rate. Retinoid-binding proteins are designed to maintain retinoids in a stable, non-degradable form, which is crucial for their role in cellular processes such as gene regulation and vision. The longevity and stability of these proteins are essential for the proper transport and regulation of retinoids within cells and tissues. Their interaction with hydrophobic molecules like retinoids helps maintain their structure and reduces susceptibility to degradation. The stability and resistance to degradation are closely related to their functional roles, ensuring they have a longer half-life to perform their critical functions in the body. Examples of retinoid-binding proteins are retinol-binding protein (RBP), cellular retinol-binding protein (CRBP), cellular retinoic acid-binding protein (CRABP) and retinal-binding protein (RBP (retinal)). For all these proteins, their interaction with hydrophobic molecules (whether lipids or retinoids) can contribute to stability.

While a long half-life is not an inherent feature of all annexins, FABPs, or retinoid-binding proteins, factors such as stable folding, the absence of strong degradation signals, interactions with hydrophobic molecules, and their functional roles contribute to their stability and potential for a longer half-life. The specific characteristics of each protein type can influence its overall stability and half-life in a cellular or recombinant expression system.

### Example 23C: Animal-Derived Annexins, Fatty Acid-Binding Proteins (FABPs), and Retinoid-Binding Proteins (RBPs): Why are these Proteins not Particularly Toxic to Yeast Cells?

These proteins are expressed at high levels and the yeast still typically grows well even though the high amount of production of animal-derived recombinant proteins, does slow down the yeast to typically 80-90% of the unengineered yeast and in some cases more or less than that. It is possible that these animal-derived proteins could be less toxic to yeast cells compared to the majority of other proteins.

Animal-derived proteins might not interfere with yeast cellular machinery or structures, avoiding potential disruption of vital processes. In contrast, proteins that closely resemble native yeast proteins might inadvertently interfere with normal cellular functions if they misfold or compete with native proteins.

These particular animal-derived proteins have specific functions, such as binding hydrophobic molecules and participating in particular metabolic pathways. These functions might be relatively benign to yeast cells, causing less cellular stress or toxicity compared to proteins that could disrupt essential yeast processes.

Proteins that are less similar to native yeast proteins might avoid forming aggregates or misfolded species that can be toxic to the cell. Properly folded proteins with stable structures are less likely to cause cellular stress or toxicity

Yeast cells may activate stress responses in reaction to misfolded proteins or those that interfere with cellular processes. Animal-derived proteins, if they fold correctly and do not trigger such stress responses, may be less toxic.

Yeast might have evolved to tolerate or manage certain types of stress better than others. Animal-derived proteins that do not activate or overwhelm yeast's stress response systems are less likely to be toxic.

While yeast lacks a true immune system, it has mechanisms to detect and respond to unusual proteins. Proteins that are markedly different from yeast proteins might avoid triggering cellular responses that could be harmful.

The lower toxicity of these animal-derived proteins in yeast could result from their unique functional roles, proper folding, and minimal interaction with yeast's stress response systems. Their distinct nature compared to native yeast proteins likely helps them avoid disrupting essential cellular processes, contributing to their successful expression without significant toxicity. In addition, recombinant yeast systems can be engineered to optimize expression conditions for specific proteins, including those from animals. Such controlled environments can minimize potential toxicity by carefully managing expression levels and conditions.

The lower toxicity of these animal-derived proteins in yeast may also be related to the abundance of certain amino acids. For example, we have observed that our best expressing annexins and FABPs have lower histidine and proline content compared to animal-derived proteins that express less well. Certain amino acids, such as histidine, are considered reactive, while others, such as proline, are associated with flexible structures, which may negatively affect capacity for high expression.

Protein expression levels in S. cerevisiae strains with HapAmp integrations vary widely depending on the protein being expressed. While transcription, translation, and degradation rates are considered key factors of gene and protein expression level, the extent to which these factors depend on the gene or amino acid sequence are less well understood. We analyzed the sequences of our best and worst performing proteins in S. cerevisiae, as scored from SDS-PAGE gels. Our findings indicate that hydrophobicity, predicted stability, predicted secondary structure, and specific amino acid content affect a protein's capacity for high expression, though these properties alone are not sufficient to fully predict expression levels.

References corresponding to Example 23, each of which are incorporated herein by reference in its entirety for all purposes: Lizarbe et al., Annexin-Phospholipid Interactions. Functional Implications, Int. J. Mol. Schi. 14(2), (2013); Moss & Morgan, The annexins, Genome Bio. 5, 219(2004); Parks et al., The FAR protein family of parasitic nematodes, PLoS Pathogens, 2022.

### Example 24

This example provides estimated expression levels of different animal proteins for different expression methods based on the findings of the above examples.

TABLE 10
Estimated expression levels of different animal proteins for different expression methods.

| | | Expression level | | | | | |
|---|---|---|---|---|---|---|---|
| | | Initial | Expression Method | | | | |
| | | Screen | HapA | | Safe | | |
| | | 2-micron | mp | POT1 | Harbor | | Size |
| PROTEIN | Animal | screen | Method | Method | Method | Accession # | (KDa) |
| Annexin A5 | Chicken | 3 | 2 | — | 3 | P17153 | 36 |
| DESTRIN | Chicken | 3 | 1 | 3 | 2 | P18359 | 19 |
| PGK1 | Chicken | 3 | — | — | — | P51903 | 45 |
| Phosphoglucomutase | Chicken | 3 | — | — | — | F1NN63 | 62 |
| Glucose-6-phos isomerase (PGI1) | Chicken | 3 | — | — | — | F1NIJ6 | 60 |
| Annexin A5 | Chicken | 3 | 2 | — | — | F1NJI0 | 37 |
| Annexin A4 | Chicken | 3 | 2 | — | — | XP_040545559.1 | 35 |
| Annexin A3 | Lamb | 3 | 3 | 3 | 3 | XP_004009983.1 | 36 |
| Annexin A6 isoform X1 | Lamb | 3 | — | 2 | — | XP_004009036.1 | 76 |
| Transgelin | Lamb | 3 | — | 3 | — | XP_004016100.2 | 23 |
| TPM1 | Lamb | 3 | — | — | — | ACB97628.1 | 33 |
| Annexin A4 | Lamb | 3 | — | — | — | XP_004005861.1 | 36 |
| Annexin A8 | Red Deer | 3 | 2 | — | — | XP 043782191.1 | 36.4 |
| FABP4, fatty acid binding protein 4 | Red Deer | 3 | 2 | — | — | ABR68240.1 | 14.6 |
| Annexin A3 | Red Deer | 3 | — | — | — | XP_043762326.1 | 35.9 |
| Annexin A4 | Red Deer | 3 | 2 | — | — | XP_043773485.1 | 35.5 |
| Annexin A1 | Red Deer | 3 | — | — | — | XP_043747439.1 | 38.5 |
| Annexin A2 | Red Deer | 2 | — | — | — | XP_043774895.1 | 37.7 |
| KLHL40, kelch-like protein 40 | Red Deer | 2 | — | — | — | XP_043742651.1 | 69.8 |
| TPM2, tropomyosin beta chain | Red Deer | 2 | — | — | — | XP_043746517.1 | 33 |
| isoform X1 | | | | | | | |
| Annexin A6 isoform X1 | Red Deer | 2 | — | — | — | XP_043769272.1 | 75 |
| Annexin A6 isoform X2 | Red Deer | 2 | — | — | — | XP_043769273.1 | 74.4 |
| TPM3, tropomyosin alpha-3 chain | Red Deer | 2 | — | — | — | XP_043731618.1 | 32.8 |
| isoform X1 | | | | | | | |
| Annexin A13 isoform X3 | Red Deer | 2 | — | — | — | XP_043736164.1 | 36.7 |
| TROPOMYOSIN alpha-1 | Chicken | 2 | 1 | 2 | 1 | P04268 | 33 |
| chain isoform X1 | | | | | | | |
| Tropomyosin beta chain/Tropomyosin-2 | Chicken | 2 | — | — | — | P19352 | 33 |
| Transgelin | Chicken | 2 | 1 | — | 2 | P19966 | 22 |
| RADIXIN | Chicken | 2 | — | 1 | — | F1NQD9 | 69 |
| COFILIN-2 | Chicken | 2 | — | 2 | — | P21566 | 19 |
| CAVEOLIN-3 | Chicken | 2 | — | 0 | — | NP_989701.2 | 18 |
| CONNECTIN/TITIN | Chicken | 2 | — | 0 | — | 042096 | 24 |
| PROFILIN-2 | Chicken | 2 | — | 2 | — | Q5ZL50 | 15 |
| Annexin A2 | Chicken | 2 | — | — | — | P17785 | 39 |
| Titin 1-94AA | Chicken | 2 | — | — | — | A6BM71 | 21 |
| Titin 4622-4896AA | Chicken | 2 | — | — | — | A6BM71 | 32 |
| Dystrophin | Chicken | 2 | — | — | — | P11533 | 28 |
| Pyruvate kinase domain | Chicken | 2 | — | — | — | P00548 | 58 |
| TITIN ISOFORM CH12 | Chicken | 2 | — | — | — | A6BLM7 | 60 |
| Myosin light chain 1, skeletal muscle | Chicken | 2 | — | — | — | PO2604 | 21 |
| Beta-enolase | Chicken | 2 | — | — | — | P07322 | 47 |
| Fructose-bisphos ald-A | Chicken | 2 | — | — | — | XP_040512782.1 | 31 |
| Annexin A2 | Lamb | 2 | — | — | — | A2SW69 | 38 |
| Annexin A1 | Lamb | 2 | — | — | — | XP_004004354.1 | 39 |
| Smoothelin-like protein-2 | Lamb | 2 | — | — | — | XP_004013322.1 | 38 |
| Myozenin-3 | Lamb | 2 | — | — | — | ADQ28100.1 | 27 |
| Destrin | Lamb | 2 | — | — | — | W5Q831 | 20 |
| Profilin-1 like | Lamb | 2 | — | — | — | XP_004009583.1 | 20 |
| Cofilin-2 | Cow | 2 | — | — | — | Q148F1.1 | 19 |
| PARK7 protein | Cow | 2 | — | — | — | AAI02708.1 | 20 |
| Annexin A2 | Cow | 2 | — | — | — | P04272.2 | 39 |
| Tropomyosin alpha-1 chain | Cow | 2 | — | — | — | NP_001013608.1 | 33 |
| ANKRD2, ankyrin repeat domain- | Red Deer | 1 | — | — | — | XP_043782106.1 | 37 |
| containing protein 2 | | | | | | | |
| Troponin T, Slow skeletal | Chicken | 1 | — | — | — | Q98916 | 32 |
| MYOSIN LIGHT CHAIN 3 | Chicken | 1 | — | — | — | P02605 | 17 |
| Gelsolin | Chicken | 1 | — | — | — | O93510 | 86 |
| Annexin A13 isoform X1 | Chicken | 1 | — | — | — | A0A1D5NUB1 | 35 |
| Myosin Motor Domain | Chicken | 1 | — | — | — | F1P3X1 | 79 |
| SH3 Domain | Chicken | 1 | — | — | — | A0A3Q2U8G4 | 28 |
| Gamma-sarcoglycan isoform X3 | Chicken | 1 | — | — | — | XP_004938861.1 | 32 |
| TROPONIN T, FAST SKELETAL | Chicken | 1 | — | — | — | NP_990253.1 | 34 |
| Annexin A1 (fragment) | Chicken | 1 | — | — | — | Q92108 | 15 |
| Beta-actin | Chicken | 1 | — | — | — | P60706 | 42 |
| DESMIN | Chicken | 1 | — | — | — | PO2542 | 53 |
| Coronin | Chicken | 1 | — | — | — | F1NXA5 | 53 |
| Pyruvate Kinase | Chicken | 1 | — | — | — | P00548 | 58 |
| Four and half LIM domains (FHL)-1 | Lamb | 1 | — | — | — | ACV04827.1 | 34 |
| Alpha actinin-3 | Lamb | 1 | — | — | — | XP_004019751.2 | 103 |
| Cofilin-2 | Lamb | 1 | — | — | — | XP_004017937.1 | 17 |
| Telethonin | Lamb | 1 | — | — | — | XP_004012895.1 | 19 |
| Profilin-1 | Lamb | 1 | — | — | — | XP_027830885.1 | 19 |
| Fibulin-5 | Lamb | 1 | — | — | — | XP_004018000.1 | 50 |
| CysGly-rich prot3 | Cow | 1 | — | — | — | Q4UOT9.1 | 21 |
| Alpha-actinin-3 | Cow | 1 | — | — | — | G3X711 | 103 |
| Myozenin 1 | Turkey | 1 | — | — | — | XP_010712691.1 | 33 |
| Troponin C, skeletal muscle | Pig | 1 | — | — | — | NP_001001862.1 | 18 |
| Annexin A10 | Red Deer | 1 | — | — | — | XP_043747177.1 | 36.1 |
| Annexin A11 | Red Deer | 1 | — | — | — | XP_043783035.1 | 56.1 |
| Annexin A13 isoform X1 | Red Deer | 1 | — | — | — | XP_043736162.1 | 41.4 |
| Annexin A13 isoform X2 | Red Deer | 1 | — | — | — | XP_043736163.1 | 39.9 |
| Annexin A5 isoform X2 | Red Deer | 1 | — | — | — | XP_043726894.1 | 39.4 |
| Annexin A9 isoform X1 | Red Deer | 1 | — | — | — | XP_043734015.1 | 54.9 |
| MYL4, myosin light chain 3 | Red Deer | 1 | — | — | — | XP_043741994.1 | 25.4 |
| Creatine Kinase M-type | Chicken | 0 | — | — | — | P00565 | 43 |
| Fascin | Chicken | 0 | — | — | — | D5LPR1 | 54 |
| Nebulin-like domain | Chicken | 0 | — | — | — | Q9DEH4 | 43 |
| Annexin A6 | Chicken | 0 | — | — | — | P51901 | 75 |
| TROPONIN I, SLOW SKELETAL | Chicken | 0 | — | — | — | XP_419242.3 | 22 |
| TITIN FRAG 66 kDa | Chicken | 0 | — | — | — | Q90720 | 66 |
| TITIN FRAG 91 kDa | Chicken | 0 | — | — | — | Q07784 | 91 |
| Troponin C, skeletal, Isoform 1 | Chicken | 0 | — | — | — | P02588 | 18 |
| Troponin C, Isoform 2 | Chicken | 0 | — | — | — | P09860 | 18 |
| Fast skeletal myosin light chain-2 | Lamb | 0 | — | — | — | ACM43300.1 | 50 |
| Galectin-1 | Lamb | 0 | — | — | — | AAT38511.1 | 15 |
| Myotilin isoform-1 | Lamb | 0 | — | — | — | XP_004008869.1/ | 56 |
| | | | | | | W5Q1Q5\| | |
| actin-alpha skeletal muscle | Lamb | 0 | — | — | — | XP_004021390.1 | 46 |
| Nebulin-like domain AA2625-2961 | Lamb | 0 | — | — | — | XP_042099807.1 | 39 |
| Titin Protein Kinase Domain | Lamb | 0 | — | — | — | XP_042100821.1 | 30 |
| Titin M10-domain | Lamb | 0 | — | — | — | XP_042100821.1 | 29 |
| M-protein, striated muscle | Salmon | 0 | — | — | — | A0A1S3L6D5 | 153 |
| TNNC2, troponin C, skeletal muscle | Red Deer | 0 | — | — | — | XP_043740492.1 | 18 |
| AHSG, alpha-2-HS-glycoprotein | Red Deer | 0 | — | — | — | XP_043729914.1 | 38 |
| MYL3, myosin light chain 3 | Red Deer | 0 | — | — | — | XP_043741994.1 | 25.4 |
| LMOD3, leiomodin-3 | Red Deer | 0 | — | — | — | XP_043741776.1 | 65.2 |
| TNNI2, troponin I, fast skeletal muscle | Red Deer | 0 | — | — | — | XP_043779185.1 | 21.3 |
| MYOZ1, myozenin-1 | Red Deer | 0 | — | — | — | XP_043782845.1 | 32 |
| VGLL2, transcription cofactor vestigial-like | Red Deer | 0 | — | — | — | XP_043746373.1 | 34 |
| protein 2 isoform X1 | | | | | | | |
| SERPINA3-1, serpin A3-1 | Red Deer | 0 | — | — | — | XP_043778278.1 | 46 |
| TMOD4, tropomodulin-4 | Red Deer | 0 | — | — | — | XP_043732112.1 | 39.2 |
| Annexin A3 | Water Buffalo | — | — | — | — | XP_006053803.3 | 36.1 |
| Annexin A5 isoform X2 | Water Buffalo | — | — | — | — | XP_006043126.1 | 36.1 |
| Annexin A6 isoform X1 | Water Buffalo | — | — | — | — | XP_006041264.1 | 75.8 |
| Annexin A8 | Water Buffalo | — | — | — | — | XP_006066854.2 | 36.9 |
| FABP9, fatty acid-binding protein 9 | Water Buffalo | — | — | — | — | XP_006068110.1 | 14.9 |
| FABP12, fatty acid-binding protein 12 | Water Buffalo | — | — | — | — | XP_006068112.3 | 15.9 |
| FABP5X1, fatty acid-binding protein 5 | Water Buffalo | — | — | — | — | XP_006068107.1 | 15.1 |
| isoform X1 | | | | | | | |
| RBP2, retinol-binding protein 2 | Water Buffalo | — | — | — | — | XP_006070028.2 | 15.7 |

TABLE 10. Estimated expression levels of different animal proteins for different expression methods. The expression level is ranked based on three different expression systems; 2-micron, HapAmp, and Safe-Harbor expression systems. The 2-micron and Safe-harbor expression are ranked using a qualitative numerical approach (0-3, 0=No expression, 1=low, 2=medium, and 3=Good). The HapAmp scoring is based on a quantitative approach using % of protein based on mass spectrometry total protein approach (TPA), >20%=3, >10%=2, >0%=1. Accession #indicates NCBI/Uniprot accession number. W indicates the calculated molecular weight in kilodaltons (kDa).

### Example 25

This example provides additional example proteins to be used in any of the methods and compositions discussed herein.

TABLE 11
Example proteins

| Protein name | Accession # | Other detail | Species |
|---|---|---|---|
| Cellular retinoic acid | NP_001025710 | cellular retinoic acid-binding | Gallus gallus |
| binding protein 1 | | protein 1 | |
| Cellular retinoic acid | NP_001121964 | cellular retinoic acid-binding | Sus scrofa |
| binding protein 1 | | protein 1 | |
| Cellular retinoic acid | P62964 | Cellular retinoic acid-binding | Bos taurus |
| binding protein 1 | | protein 1 | |
| Cellular retinoic acid | NP_001134198 | cellular retinoic acid-binding | Salmo salar |
| binding protein 1 | | protein 1 | |
| Cellular retinoic acid | EOA95424 | Cellular retinoic acid-binding | Anas |
| binding protein 1 | | protein 1, partial | platyrhynchos |
| Cellular retinoic acid | AAV84003 | cellular retinoic acid binding | Bos taurus |
| binding protein 1 | | protein 1 | |
| Cellular retinoic acid | XP_019474825 | cellular retinoic acid-binding | Meleagris |
| binding protein 1 | | protein 1, partial | gallopavo |
| Cellular retinoic acid | ABQ66017 | cellular retinoic acid binding | Sus scrofa |
| binding protein 1 | | protein 1 | |
| Cellular retinoic acid | XP_045564476 | cellular retinoic acid-binding | Salmo salar |
| binding protein 1 | | protein 1 | |
| Cellular retinoic acid | Q5PXY7 | Cellular retinoic acid-binding | Bos taurus |
| binding protein 2 | | protein 2 | |
| Cellular retinoic acid | P30370 | Cellular retinoic acid-binding | Gallus gallus |
| binding protein 2 | | protein 2 | |
| Cellular retinoic acid | ACZ57803 | cellular retinoic acid binding | Sus scrofa |
| binding protein 2 | | protein 2, partial | |
| Cellular retinoic acid | AAV84002 | cellular retinoic acid binding | Bos taurus |
| binding protein 2 | | protein 2 | |
| Cellular retinoic acid | NP_001157981 | cellular retinoic acid-binding | Sus scrofa |
| binding protein 2 | | protein 2 | |
| Cellular retinol | ACY07984 | cellular retinol-binding protein | Sus scrofa |
| binding protein 1 | | 1, partial | |
| Cellular retinol | AAQ07459 | cellular retinol binding protein | Bos taurus |
| binding protein 1 | | 1 | |
| Cellular retinol | NP_001026959 | retinol-binding protein 1 | Sus scrofa |
| binding protein 1 | | | |
| Cellular retinol | NP_001020514 | retinol-binding protein 1 | Bos taurus |
| binding protein 1 | | | |
| Cellular retinol | ACZ57801 | cellular retinol binding protein | Sus scrofa |
| binding protein 2 | | 2, partial | |
| Cellular retinol | NP_001138695 | retinol-binding protein 5 | Sus scrofa |
| binding protein 5 | | | |
| Cellular retinol | ABQ02505 | cellular retinol binding protein | Sus scrofa |
| binding protein 5 | | 5, partial | |
| Cellular retinol | ACZ57802 | cellular retinol binding protein | Sus scrofa |
| binding protein 7 | | 7, partial | |
| Cellular retinol | ABQ02503 | cellular retinol binding protein | Sus scrofa |
| binding protein 7 | | 7 | |
| Fatty acid binding | DAA24570 | TPA: fatty acid-binding protein, | Bos taurus |
| protein 1 | | liver | |
| Fatty acid binding | XP_002710703 | fatty acid-binding protein 12 | Oryctolagus |
| protein 12 | | | cuniculus |
| Fatty acid binding | DAA28927 | TPA: fatty acid-binding protein, | Bos taurus |
| protein 2 | | intestinal, partial | |
| Fatty acid binding | ABW37177 | fatty acid binding protein 2 | Sus scrofa |
| protein 2 | | | |
| Fatty acid binding | P10790 | Fatty acid-binding protein, heart | Bos taurus |
| protein 3 | | | |
| Fatty acid binding | AAT72764 | fatty acid binding protein 3, | Sus scrofa |
| protein 3 | | partial | |
| Fatty acid binding | ACN76574 | fatty acid-binding protein 3, | Sus scrofa |
| protein 3 | | partial | |
| Fatty acid binding | AID56662 | fatty acid binding protein 3, | Bos taurus |
| protein 3 | | partial | |
| Fatty acid binding | ACH85195 | fatty acid binding protein 3, | Sus scrofa |
| protein 3 | | partial | |
| Fatty acid binding | AFS28890 | fatty acid binding protein 4, | Ovis aries |
| protein 4 | | partial | |
| Fatty acid binding | ABK58143 | fatty acid binding protein 4 | Sus scrofa |
| protein 4 | | | |
| Fatty acid binding | AFS28889 | fatty acid binding protein 4, | Ovis aries |
| protein 4 | | partial | |
| Fatty acid binding | O97788 | Fatty acid-binding protein, | Sus scrofa |
| protein 4 | | adipocyte | |
| Fatty acid binding | ABK58163 | fatty acid binding protein 4 | Sus scrofa |
| protein 4 | | | |
| Fatty acid binding | WNH24550 | fatty acid binding protein 4, | Bos taurus |
| protein 4 | | partial | |
| Fatty acid binding | ABK58162 | fatty acid binding protein 4 | Sus scrofa |
| protein 4 | | | |
| Fatty acid binding | ABK58146 | fatty acid binding protein 4 | Sus scrofa |
| protein 4 | | | |
| Fatty acid binding | ABK58151 | fatty acid binding protein 4 | Sus scrofa |
| protein 4 | | | |
| Fatty acid binding | ABK58145 | fatty acid binding protein 4 | Sus scrofa |
| protein 4 | | | |
| Fatty acid binding | ABK58156 | fatty acid binding protein 4 | Sus scrofa |
| protein 4 | | | |
| Fatty acid binding | ABR68240 | fatty acid binding protein 4 | Cervus elaphus |
| protein 4 | | | |
| Fatty acid binding | ABK58144 | fatty acid binding protein 4 | Sus scrofa |
| protein 4 | | | |
| Fatty acid binding | ABK58147 | fatty acid binding protein 4 | Sus scrofa |
| protein 4 | | | |
| Fatty acid binding | ABK58149 | fatty acid binding protein 4 | Sus scrofa |
| protein 4 | | | |
| Fatty acid binding | ABK58165 | fatty acid binding protein 4 | Sus scrofa |
| protein 4 | | | |
| Fatty acid binding | ABK58160 | fatty acid binding protein 4 | Sus scrofa |
| protein 4 | | | |
| Fatty acid binding | ABK58164 | fatty acid binding protein 4 | Sus scrofa |
| protein 4 | | | |
| Fatty acid binding | WNH24551 | fatty acid binding protein 4, | Bos taurus |
| protein 4 | | partial | |
| Fatty acid binding | ABK58159 | fatty acid binding protein 4 | Sus scrofa |
| protein 4 | | | |
| Fatty acid binding | ABK58158 | fatty acid binding protein 4 | Sus scrofa |
| protein 4 | | | |
| Fatty acid binding | ACL80572 | fatty acid binding protein 4, | Anas |
| protein 4 | | partial | platyrhynchos |
| Fatty acid binding | AFS28891 | fatty acid binding protein 4, | Ovis aries |
| protein 4 | | partial | |
| Fatty acid binding | ABK58148 | fatty acid binding protein 4, | Sus scrofa |
| protein 4 | | partial | |
| Fatty acid binding | AFU10495 | fatty acid binding protein 4, | Ovis aries |
| protein 4 | | partial | |
| Fatty acid binding | ABK58155 | fatty acid binding protein 4, | Sus scrofa |
| protein 4 | | partial | |
| Fatty acid binding | ABK58157 | fatty acid binding protein 4 | Sus scrofa |
| protein 4 | | | |
| Fatty acid binding | AFU10497 | fatty acid binding protein 4, | Ovis aries |
| protein 4 | | partial | |
| Fatty acid binding | AFU10496 | fatty acid binding protein 4, | Ovis aries |
| protein 4 | | partial | |
| Fatty acid binding | ABK58152 | fatty acid binding protein 4 | Sus scrofa |
| protein 4 | | | |
| Fatty acid binding | ABK58150 | fatty acid binding protein 4 | Sus scrofa |
| protein 4 | | | |
| Fatty acid binding | AFS28888 | fatty acid binding protein 4, | Ovis aries |
| protein 4 | | partial | |
| Fatty acid binding | ABK58161 | fatty acid binding protein 4 | Sus scrofa |
| protein 4 | | | |
| Fatty acid binding | ABK58153 | fatty acid binding protein 4 | Sus scrofa |
| protein 4 | | | |
| Fatty acid binding | AFU10494 | fatty acid binding protein 4, | Ovis aries |
| protein 4 | | partial | |
| Fatty acid binding | AFS28887 | fatty acid binding protein 4, | Ovis aries |
| protein 4 | | partial | |
| Fatty acid binding | AID56663 | fatty acid binding protein 4, | Bos taurus |
| protein 4 | | partial | |
| Fatty acid binding | ABY84512 | fatty acid binding protein 5 | Sus scrofa |
| protein 5 | | | |
| Fatty acid binding | ABF71384 | fatty acid binding protein 5 | Sus scrofa |
| protein 5 | | | |
| Fatty acid binding | ACA05020 | fatty acid binding protein 5 | Sus scrofa |
| protein 5 | | | |
| Fatty acid binding | ADG45270 | fatty acid binding protein 5, | Sus scrofa |
| protein 5 | | partial | |
| Fatty acid binding | ACA05015 | fatty acid binding protein 5 | Sus scrofa |
| protein 5 | | | |
| Fatty acid binding | ADG45264 | fatty acid binding protein 5, | Sus scrofa |
| protein 5 | | partial | |
| Fatty acid binding | ADG45280 | fatty acid binding protein 5, | Sus scrofa |
| protein 5 | | partial | |
| Fatty acid binding | ADG45263 | fatty acid binding protein 5, | Sus scrofa |
| protein 5 | | partial | |
| Fatty acid binding | ADG45256 | fatty acid binding protein 5, | Sus scrofa |
| protein 5 | | partial | |
| Fatty acid binding | ADG45259 | fatty acid binding protein 5, | Sus scrofa |
| protein 5 | | partial | |
| Fatty acid binding | ACA05030 | fatty acid binding protein 5 | Sus scrofa |
| protein 5 | | | |
| Fatty acid binding | ACA05029 | fatty acid binding protein 5 | Sus scrofa |
| protein 5 | | | |
| Fatty acid binding | NP_776740 | fatty acid-binding protein 5 | Bos taurus |
| protein 5 | | | |
| Fatty acid binding | ADG45282 | fatty acid binding protein 5, | Sus scrofa |
| protein 5 | | partial | |
| Fatty acid binding | ADG45268 | fatty acid binding protein 5, | Sus scrofa |
| protein 5 | | partial | |
| Fatty acid binding | ADG45277 | fatty acid binding protein 5, | Sus scrofa |
| protein 5 | | partial | |
| Fatty acid binding | ACA05016 | fatty acid binding protein 5 | Sus scrofa |
| protein 5 | | | |
| Fatty acid binding | AAY34278 | fatty acid binding protein 5, | Sus scrofa |
| protein 5 | | partial | |
| Fatty acid binding | ADG45269 | fatty acid binding protein 5, | Sus scrofa |
| protein 5 | | partial | |
| Fatty acid binding | AAX14717 | fatty acid binding protein 5 | Sus scrofa |
| protein 5 | | | |
| Fatty acid binding | ADG45278 | fatty acid binding protein 5, | Sus scrofa |
| protein 5 | | partial | |
| Fatty acid binding | ADG45274 | fatty acid binding protein 5, | Sus scrofa |
| protein 5 | | partial | |
| Fatty acid binding | ADG45273 | fatty acid binding protein 5, | Sus scrofa |
| protein 5 | | partial | |
| Fatty acid binding | NP_001034835 | fatty acid-binding protein 5 | Sus scrofa |
| protein 5 | | | |
| Fatty acid binding | ABY84508 | fatty acid binding protein 5 | Sus scrofa |
| protein 5 | | | |
| Fatty acid binding | ACA05024 | fatty acid binding protein 5 | Sus scrofa |
| protein 5 | | | |
| Fatty acid binding | ACA05014 | fatty acid binding protein 5 | Sus scrofa |
| protein 5 | | | |
| Fatty acid binding | ACM43302 | fatty acid binding protein 5 | Ovis aries |
| protein 5 | | | |
| Fatty acid binding | ACA05027 | fatty acid binding protein 5 | Sus scrofa |
| protein 5 | | | |
| Fatty acid binding | ACA05026 | fatty acid binding protein 5 | Sus scrofa |
| protein 5 | | | |
| Fatty acid binding | ADG45281 | fatty acid binding protein 5, | Sus scrofa |
| protein 5 | | partial | |
| Fatty acid binding | ABY84509 | fatty acid binding protein 5 | Sus scrofa |
| protein 5 | | | |
| Fatty acid binding | ADG45260 | fatty acid binding protein 5, | Sus scrofa |
| protein 5 | | partial | |
| Fatty acid binding | ADG45279 | fatty acid binding protein 5, | Sus scrofa |
| protein 5 | | partial | |
| Fatty acid binding | ADG45258 | fatty acid binding protein 5, | Sus scrofa |
| protein 5 | | partial | |
| Fatty acid binding | ABY84510 | fatty acid binding protein 5 | Sus scrofa |
| protein 5 | | | |
| Fatty acid binding | ABY84515 | fatty acid binding protein 5 | Sus scrofa |
| protein 5 | | | |
| Fatty acid binding | ADG45285 | fatty acid binding protein 5, | Sus scrofa |
| protein 5 | | partial | |
| Fatty acid binding | ADG45271 | fatty acid binding protein 5, | Sus scrofa |
| protein 5 | | partial | |
| Fatty acid binding | ACA05028 | fatty acid binding protein 5 | Sus scrofa |
| protein 5 | | | |
| Fatty acid binding | ACA05021 | fatty acid binding protein 5 | Sus scrofa |
| protein 5 | | | |
| Fatty acid binding | ADG45257 | fatty acid binding protein 5, | Sus scrofa |
| protein 5 | | partial | |
| Fatty acid binding | ABY84513 | fatty acid binding protein 5 | Sus scrofa |
| protein 5 | | | |
| Fatty acid binding | ADG45266 | fatty acid binding protein 5, | Sus scrofa |
| protein 5 | | partial | |
| Fatty acid binding | ABB46354 | fatty acid binding protein 5, | Ovis aries |
| protein 5 | | partial | |
| Fatty acid binding | ADG45286 | fatty acid binding protein 5, | Sus scrofa |
| protein 5 | | partial | |
| Fatty acid binding | ADG45267 | fatty acid binding protein 5, | Sus scrofa |
| protein 5 | | partial | |
| Fatty acid binding | ACA05023 | fatty acid binding protein 5 | Sus scrofa |
| protein 5 | | | |
| Fatty acid binding | ADG45262 | fatty acid binding protein 5, | Sus scrofa |
| protein 5 | | partial | |
| Fatty acid binding | ABY84511 | fatty acid binding protein 5 | Sus scrofa |
| protein 5 | | | |
| Fatty acid binding | ADG45272 | fatty acid binding protein 5, | Sus scrofa |
| protein 5 | | partial | |
| Fatty acid binding | ADG45276 | fatty acid binding protein 5, | Sus scrofa |
| protein 5 | | partial | |
| Fatty acid binding | ADG45283 | fatty acid binding protein 5, | Sus scrofa |
| protein 5 | | partial | |
| Fatty acid binding | ABF71383 | fatty acid binding protein 5, | Sus scrofa |
| protein 5 | | partial | |
| Fatty acid binding | ABY84516 | fatty acid binding protein 5 | Sus scrofa |
| protein 5 | | | |
| Fatty acid binding | ADG45275 | fatty acid binding protein 5, | Sus scrofa |
| protein 5 | | partial | |
| Fatty acid binding | ACA05017 | fatty acid binding protein 5 | Sus scrofa |
| protein 5 | | | |
| Fatty acid binding | ABY84507 | fatty acid binding protein 5 | Sus scrofa |
| protein 5 | | | |
| Fatty acid binding | ABY84514 | fatty acid binding protein 5 | Sus scrofa |
| protein 5 | | | |
| Fatty acid binding | ADG45261 | fatty acid binding protein 5, | Sus scrofa |
| protein 5 | | partial | |
| Fatty acid binding | ACA05018 | fatty acid binding protein 5 | Sus scrofa |
| protein 5 | | | |
| Fatty acid binding | ADG45284 | fatty acid binding protein 5, | Sus scrofa |
| protein 5 | | partial | |
| Fatty acid binding | ACA05025 | fatty acid binding protein 5 | Sus scrofa |
| protein 5 | | | |
| Fatty acid binding | ACA05019 | fatty acid binding protein 5 | Sus scrofa |
| protein 5 | | | |
| Fatty acid binding | ADG45265 | fatty acid binding protein 5, | Sus scrofa |
| protein 5 | | partial | |
| Fatty acid binding | ABY84517 | fatty acid binding protein 5 | Sus scrofa |
| protein 5 | | | |
| Fatty acid binding | DAA27219 | TPA: gastrotropin | Bos taurus |
| protein 6 | | | |
| Fatty acid binding | AAS77823 | fatty acid binding protein 6, | Sus scrofa |
| protein 6 | | partial | |
| Fatty acid binding | DAA26338 | TPA: fatty acid-binding protein, | Bos taurus |
| protein 7 | | brain | |
| Lipocalin 1 | XP_010808945 | lipocalin-1 | Bos taurus |
| Lipocalin 1 | P53715 | Lipocalin-1 | Sus scrofa |
| Lipocalin 15 | XP_004945781 | lipocalin-15 | Gallus gallus |
| Lipocalin 2 | CAX36897 | lipocalin 2, partial | Sus scrofa |
| Lipocalin 5 | CAX36898 | lipocalin 5, partial | Sus scrofa |
| Lipocalin 6 | CAX36899 | lipocalin 6, partial | Sus scrofa |
| Lipocalin 8 | CAX36900 | lipocalin 8, partial | Sus scrofa |
| Retinol binding | P02694 | Retinol-binding protein 1 | Bos taurus |
| protein 1 | | | |
| Retinol binding | EOA94968 | Retinol-binding protein 1, | Anas |
| protein 1 | | partial | platyrhynchos |
| Retinol binding | XP_043730769 | retinol-binding protein 2 | Cervus elaphus |
| protein 2 | | | |
| Retinol binding | QST77395 | retinol-binding protein 2, partial | Oryctolagus |
| protein 2 | | | cuniculus |
| Retinol binding | XP_043730766 | retinol-binding protein 2 | Cervus elaphus |
| protein 2 | | | |
| Retinol binding | P50121 | Retinol-binding protein 2 | Sus scrofa |
| protein 2 | | | |
| Retinol binding | XP_043730768 | retinol-binding protein 2 | Cervus elaphus |
| protein 2 | | | |
| Retinol binding | EOA94989 | Retinol-binding protein 2, | Anas |
| protein 2 | | partial | platyrhynchos |
| Retinol binding | P12664 | Retinol-binding protein 3 | Oryctolagus |
| protein 3 | | | cuniculus |
| Retinol binding | EOA99378 | Retinol-binding protein 3, | Anas |
| protein 3 | | partial | platyrhynchos |
| Retinol binding | P12662 | Retinol-binding protein 3 | Sus scrofa |
| protein 3 | | | |
| Retinol binding | P12661 | Retinol-binding protein 3 | Bos taurus |
| protein 3 | | | |
| Retinol binding | P12663 | Retinol-binding protein 3 | Ovis aries |
| protein 3 | | | |
| Retinol binding | AGR85351 | retinol binding protein 4, partial | Sus scrofa |
| protein 4 | | | |
| Retinol binding | AGR85349 | retinol binding protein 4, partial | Sus scrofa |
| protein 4 | | | |
| Retinol binding | XP_003208093 | retinol-binding protein 4 | Meleagris |
| protein 4 | | | gallopavo |
| Retinol binding | EOB05671 | Retinol-binding protein 4, | Anas |
| protein 4 | | partial | platyrhynchos |
| Retinol binding | ABW96016 | retinol binding protein 4, partial | Sus scrofa |
| protein 4 | | | |
| Retinol binding | ACZ57804 | retinol binding protein 4, partial | Sus scrofa |
| protein 4 | | | |
| Retinol binding | AGR85348 | retinol binding protein 4, partial | Sus scrofa |
| protein 4 | | | |
| Retinol binding | AGR85350 | retinol binding protein 4, partial | Sus scrofa |
| protein 4 | | | |
| Retinol binding | ADF32075 | retinol binding protein 4, partial | Sus scrofa |
| protein 4 | | | |
| Retinol binding | XP_027823721 | retinol-binding protein 5 | Ovis aries |
| protein 5 | | | |
| Retinol binding | NP_001094662 | retinol-binding protein 5 | Bos taurus |
| protein 5 | | | |
| Retinol binding | P82708 | Retinol-binding protein 5 | Bos taurus |
| protein 5 | | | |
| Retinol binding | QDF46320 | retinol binding protein 7 | Sus scrofa |
| protein 7 | | | |
| Retinol binding | QDF46319 | retinol binding protein 7 | Sus scrofa |
| protein 7 | | | |
| Retinol binding | QDF46318 | retinol binding protein 7 | Sus scrofa |
| protein 7 | | | |

### Example 26

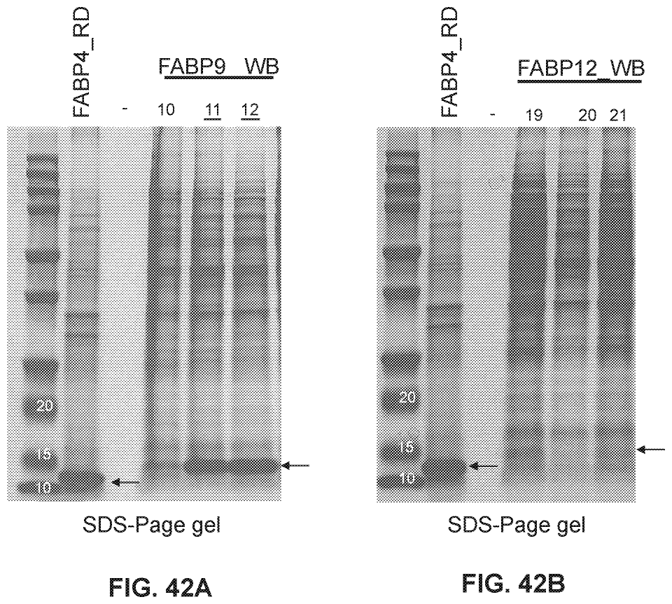

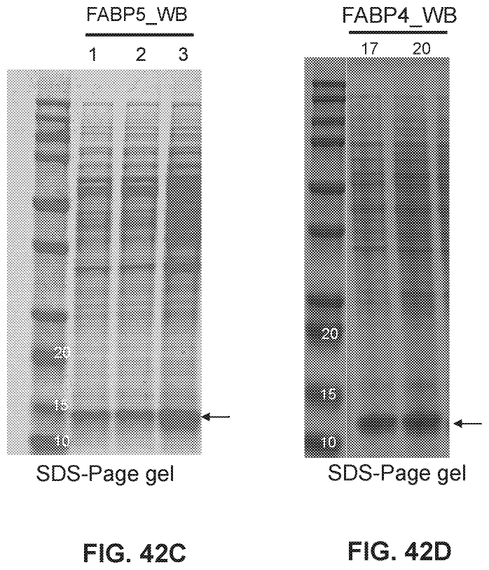

TABLE 12
Table of fatty acid-binding proteins generated and shown
in the SDS-PAGE gels of FIGs. 42A, 42B, 42C, and 42D.

| Host | Animal | FABPs |
|---|---|---|
| Saccharomyces | Red deer (RD) | FABP4 |
| cerevisiae | Water Buffalo (WB) | FABP4 |
| | | FABP5X1 |
| | | FABP9 |
| | | FABP12 |

### Example 27

This example discusses the potential advantages of using FABPs in the methods and compositions disclosed herein.

Fatty acid-binding proteins (FABPs) belong to the lipid-binding protein family. Intracellular FABPs play a role in cellular fatty acid transport and utilization, while having an indirect effect on regulating the concentration of other lipid mediators. In humans there are 10 isoforms of FABPs; FABP1, FABP2, FABP3, FABP4, FABP5, FABP6, FABP7, FABP8, FABP9, and FABP12. However, 12 FABPs have been identified in different species, but no single organism expressed all 12 FABPs. FABP9 and FABP12 are only found in mammals. FABP10 is found in chickens, amphibians, reptiles, and fish, whereas FABP 11 has only been identified in teleosts. However, FABPs are not found in Saccharomyces cerevisiae. This unique feature make them ideal for overexpression in yeast without interfering with native yeast proteins.

Because we are able to express some of these at high levels in yeast, we are able to make formulations that contain yeast and a significant portion of animal protein. The animal protein contributes to the overall nutrition needed by dogs and cats and other animals. We have seen that the total protein content increases when we have a significant overexpression of the recombinant protein, which is something that is sought after in the pet food industry. Our product is different from most other precision fermentation companies in that we mostly produce the recombinant protein intracellularly and we do not purify the protein(s) (presently).

Problem: Meat production is a major contributor to green house gas emission, thus a key player in the climate change crisis. A third of this produced meat is used for pet food production. Thus, producing animal proteins through fermentation reduces the demand for animal agriculture. Pet food companies are worried about the meat supply chain and are projecting scarcity being an issue in the next decades as the meat demand goes up. Precision fermentation offers the opportunity to craft animal proteins without harming animals. We are able to create a product that is more sustainable, while still providing high-quality nutrition to our pets.

Main features and benefits: Overexpression of heterologous proteins in yeast can cause growth/cellular defects, which is known as the protein burden. We have been able to successfully overexpress 5 different FABPs from two different animals (red deer and water buffalo) in Saccharomyces cerevisiae without observable cellular defects. We have been able to overexpress some of these FABPs at high levels >10% of heterologous protein, making them ideal candidates for the production of animal protein. It is well established that smaller proteins are more highly expressed than larger proteins. Large proteins are defined here as over 700 amino acids (~77 kDa). FABPs range from 14 kDa to 15 kDa. Overexpression of these small proteins is also beneficial for the pet food formulation of hypoallergenic food. These are normally specialty foods in which the protein source has been broken down to smaller fragments and typically elicit a reduced immune response. These small protein sources are something that pet food companies are looking for.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Every formulation, genetic construct, transformed fungi strain, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

TABLE 3
Additional proteins for use in the methods and compositions disclosed herein.

| | | Protein NCBI |
|---|---|---|
| Gene name | Protein name | accession |
| ENO3 | Beta-enolase | P07322 |
| PKM2 | Pyruvate kinase PKM | NP_990800 |
| CKM | Creatine kinase M-type | P00565 |
| TTN | Titin isoform X2 | XP_046777675 |
| PGM1 | Phosphoglucomutase-1 isoform X2 | XP_046778819 |
| MYOM2 | M-protein, striated muscle | NP_990466 |
| GAPDH | Glyceraldehyde-3-phosphate dehydrogenase | P00356 |
| ACTN2 | Alpha-actinin-2 | P20111 |
| ACTA1 | Actin, alpha skeletal muscle | P68139 |
| PGK1 | Phosphoglycerate kinase | NP_990316 |
| LDHA | L-lactate dehydrogenase A chain | P00340 |
| AGL | Glycogen debranching enzyme isoform X2 | XP_046779329 |
| TPM1 | Tropomyosin alpha- 1 chain | P04268 |
| GPI | Glucose-6- phosphate isomerase | NP_001006128 |
| PYGB | Glycogen phosphorylase, brain form | NP_001026205 |
| TPI1 | Triosephosphate isomerase | P00940 |
| HSPA8 | Heat shock cognate 71 kDa protein | O73885 |
| VIM | Vimentin | P09654 |
| ATP2A1 | Sarcoplasmic/endoplasmic reticulum calcium ATPase 1 | P13585 |
| MYOM1 | Myomesin-1 | NP_990290 |
| AK1 | Adenylate kinase isoenzyme 1 | P05081 |
| ALB | Albumin | P19121 |
| ENO1 | Alpha-enolase | P51913 |
| PGAM1 | Phosphoglycerate mutase 1 | Q5ZLN1 |
| apoAIV | Apolipoprotein A- IV precursor | NP_990269 |
| vcp | Transitional endoplasmic reticulum ATPase | NP_001038129 |
| HSP90AA1 | Heat shock protein HSP 90-alpha | P11501 |
| MYLPF | Myosin regulatory light chain 11 | P02609 |
| pfk | Phosphofructokinase | BAC20931 |
| DES | Desmin | P02542 |
| MYBPC2 | Myosin-binding protein C, fast-type | P16419 |
| HIST2H2A C | Histone H2A-IV | NP_001072943 |
| MDH2 | Malate dehydrogenase, mitochondrial isoform X2 | XP_046786190 |
| HSPB1 | Heat shock protein beta-1 | Q00649 |
| AMPD1 | AMP deaminase 1 | NP_001383553 |
| BIN1 | Myc box-dependent-interacting protein 1 isoform X36 | XP_046777429 |
| CKMT2 | Creatine kinase S-type, mitochondrial | P11009 |
| ATP5B | ATP synthase subunit beta, mitochondrial | Q5ZLC5 |
| LDB3 | LIM domain- binding protein 3 isoform X4 | XP_046775956 |
| EEF2 | Elongation factor 2 | Q90705 |
| FBP2 | Fructose-1,6-bisphosphatase isozyme 2 | XP_046791765 |
| PDIA3 | Protein disulfide-isomerase A3 | Q8JG64 |
| ACO2 | Aconitate hydratase, mitochondrial | NP_989519 |
| ATP5A1 | ATP synthase subunit alpha, mitochondrial | NP_989617 |
| GOT1 | Aspartate aminotransferase, cytoplasmic | P00504 |
| EEF1A2 | Elongation factor 1-alpha 2 isoform X1 | XP_046786707 |
| HSPA9 | Stress-70 protein, mitochondrial | Q5ZM98 |
| DJ | Protein/nucleic acid deglycase DJ-1 | NP_989916 |
| HBG2 | Hemoglobin subunit beta | NP_990820 |
| SRL | Sarcalumenin | Q90577 |
| ALDOC | Fructose- bisphosphate aldolase C | P53449 |
| HBAA | Hemoglobin subunit alpha-A | P01994 |
| MDH1 | Malate dehydrogenase, peroxisomal isoform MDH1x | NP_001303820 |
| PRDX6 | Peroxiredoxin-6 | Q5ZJF4 |
| HSPD1 | 60 kDa heat shock protein, mitochondrial | Q5ZL72 |
| TNNI2 | Troponin I, fast skeletal muscle | P68246 |
| PEBP1 | Phosphatidylethanol amine-binding protein 1 | NP_001185571 |
| EIF4A2 | Eukaryotic initiation factor 4A-II | Q8JFP1 |
| TNNC2 | Troponin C, skeletal muscle | P02588 |
| ALDH2 | Aldehyde dehydrogenase, mitochondrial | NP_001376401 |
| CFL2 | Cofilin-2 | P21566 |
| AHCY | Adenosylhomocysteinase | XP_040544353 |
| HSP70 | Heat shock 70 kDa protein | NP_001006686 |
| NPEPPS | Puromycin-sensitive aminopeptidase | XP_040547950 |
| LMNA | Lamin-A | NP_990618 |
| VCL | Vinculin | P12003 |
| NCL | Nucleolin | P15771 |
| HK1 | Hexokinase-1 isoform X1 | XP_046775670 |
| P4HB | Protein disulfide-isomerase | P09102 |
| YWHAG | 14-3-3 protein gamma | Q5F3W6 |
| PRDX3 | Thioredoxin-dependent peroxide reductase, mitochondrial isoform X2 | XP_040530814 |
| RPSA | Small ribosomal subunit protein uS2 | NP_001007824 |
| CASQ2 | Calsequestrin-2 | P19204 |
| HNRNPK | Heterogeneous nuclear ribonucleoprotein K | Q5ZIQ3 |
| RCJMB04_7e11 | Hypothetical protein RCJMB04_7e11 | CAG31511 |
| PCMT1 | Protein-L-isoaspartate(D-aspartate) O-methyltransferase | Q5F3N1 |
| PHKA1 | Phosphorylase b kinase regulatory subunit alpha, isoform X3 | XP_040525407 |
| RPS7 | 40S ribosomal protein S7 | XP_046770495 |
| NID1 | Nidogen-1 isoform X2 | XP_046770842 |
| HBAD | Hemoglobin subunit alpha-D | P02001 |
| PHKB | Phosphorylase b kinase regulatory subunit beta | NP_001007832 |
| GLO1 | Lactoylglutathione lyase isoform X2 | XP_040523530 |
| CCT8 | T-complex protein 1 subunit theta | Q6EE31 |
| OGN | Mimecan | Q9W6H0 |
| AKR1B10 | Aldo-keto reductase family 1 member B10 | NP_989960 |
| MYOZ1 | Myozenin-1 | XP_040530511 |
| RCJMB04_21l24 | Hypothetical protein RCJMB04_21l24 | CAG32278 |
| PDLIM3 | PDZ and LIM domain protein 3 | Q9PU47 |
| ME1 | NADP-dependent malic enzyme | Q92060 |
| MYOZ3 | Myozenin-3 isoform X1 | XP_040538666 |
| GDI2 | Rab GDP dissociation inhibitor beta isoform X1 | XP_046762689 |
| ANXA2 | Annexin A2 | P17785 |
| PRDX1 | Peroxiredoxin-1 | P0CB50 |
| RPS3 | Small ribosomal subunit protein uS3 | NP_001026007 |
| LOC42401 4 | Putative methyltransferase DDB_G0268948 | XP_040532202 |
| CALM | Calmodulin | P62149 |
| RCJMB04_20e18 | Hypothetical protein RCJMB04_20e18 | CAG32223 |
| LAMB1 | Laminin subunit beta-1 | Q01635 |
| KPNB1 | Importin subunit beta-1 | XP_046789508 |
| GYG1 | Glycogenin-1 isoform X3 | XP_046779945 |
| H4 | Histone cluster 1, H4-VI, germinal H4 | NP_001032934 |
| FDPS | Farnesyl pyrophosphate synthase | P08836 |
| RPS16 | 40S ribosomal protein S16 | XP_046754932 |
| CCT2 | T-complex protein 1 subunit beta isoform X1 | XP_040551714 |
| CRYL1 | Lambda-crystallin homolog | NP_001026001 |
| LMNB2 | Lamin-B2 | P14732 |
| BPGM | Bisphosphoglycerate mutase isoform X2 | XP_046763562 |
| RPLP0 | Large ribosomal subunit protein uL10 | NP_990318 |
| FABP | Fatty acid-binding protein, smooth muscle | P80565 |
| SPTAN1 | Spectrin alpha chain, non-erythrocytic 1 | P07751 |
| HSPA4 | Heat shock 70 kDa protein 4 | XP_046783064 |
| KHSRP | Far upstream element-binding protein 2 | Q8UVD9 |
| OLA1 | Obg-like ATPase 1 | Q5ZM25 |
| ACAA2 | 3-ketoacyl-CoA thiolase, mitochondrial | NP_001006571 |
| TNNC1 | Troponin C, slow skeletal and cardiac muscles | P09860 |
| RCJMB04_20m7 | Hypothetical protein RCJMB04_20m7 | CAG32246 |
| PACSIN3 | Protein kinase C and casein kinase substrate in neurons protein 3 isoform X4 | XP_046774323 |
| HADH | Hydroxyacyl-coenzyme A dehydrogenase, mitochondrial isoform X1 | XP_046771626 |
| FH | Fumarate hydratase, mitochondrial isoform a | NP_001006382 |
| PSMA1 | Proteasome subunit alpha type-1 | O42265 |
| OBSCN | Obscurin isoform X21 | XP_040519710 |
| RCJMB04_29e19 | Hypothetical protein RCJMB04_29e19 | CAG32557 |
| CS | Citrate synthase, mitochondrial | P23007 |
| RPL13 | Large ribosomal subunit protein eL13 | NP_990330 |
| ADPRHL1 | Protein ADP-ribosylarginine hydrolase-like protein 1 isoform X2 | XP_046765136 |
| RPLP2 | 60S acidic ribosomal protein P2 | XP_040529034 |
| COPS4 | COP9 signalosome complex subunit 4 | NP_001006447 |
| RDX | Radixin | Q9PU45 |
| LUM | Lumican precursor | NP_001263286 |
| HSP90B1 | Endoplasmin | P08110 |
| HADHB | Trifunctional enzyme subunit beta, mitochondrial | NP_001376414 |
| RCJMB04_18a15 | Hypothetical protein RCJMB04_18a15 | CAH65323 |
| YWHAE | 14-3-3 protein epsilon | Q5ZMT0 |
| HSPA5 | Endoplasmic reticulum chaperone BiP | Q90593 |
| GANC | Neutral alpha-glucosidase C isoform X2 | XP_046774383 |
| RCJMB04_15n19 | Hypothetical protein RCJMB04_15n19 | CAH65296 |
| HSPE1 | 10 kDa heat shock protein, mitochondrial | NP_990398 |
| HNRNPR | Heterogeneous nuclear ribonucleoprotein R isoform X1 | XP_046787904 |
| SPTBN1 | Spectrin beta chain, non-erythrocytic 1 | NP_001186354 |
| RCJMB04_19o8 | Hypothetical protein RCJMB04_19o8 | CAG32200 |
| ADSL | Adenylosuccinate lyase | P21265 |
| GSTM2 | Glutathione S-transferase 2 | P20136 |
| XPNPEP1 | Xaa-Pro aminopeptidase 1 | XP_046776777 |
| CDC37 | Hsp90 co-chaperone Cdc37 | O57476 |
| RCJMB04_23a9 | Hypothetical protein RCJMB04_23a9 | CAG32321 |
| MYLK2 | Myosin light chain kinase 2, skeletal/cardiac muscle | NP_990723 |
| CLE7 | RNA transcription, translation and transport factor protein isoforfm 2 | NP_001384251 |
| RCJMB04_23k2 | Hypothetical protein RCJMB04_23k2 | CAG32356 |
| PDCD5 | Programmed cell death protein 5 isoform X1 | XP_046781473 |
| PPP2R4 | Serine/threonine-protein phosphatase 2A activator isoform X1 | XP_046784992 |
| NPEPL1 | Probable aminopeptidase NPEPL1 | XP_040544283 |
| UBE2N | Ubiquitin- conjugating enzyme E2 N | NP_001012828 |
| PSMB1 | Proteasome subunit beta type-1 | NP_001007906 |
| RCJMB04_11a23 | Hypothetical protein RCJMB04_11a23 | CAG31783 |
| C11orf54 | Ester hydrolase C11orf54 homolog | NP_001264206 |
| YWHAZ | 14-3-3 protein zeta | Q5ZKC9 |
| PDIA6 | Protein disulfide-isomerase A6 | XP_040524769 |
| RPS25 | 40S ribosomal protein S25 | XP_040546288 |
| TPM2 | Tropomyosin beta chain | P19352 |
| SPTB | Spectrin beta chain, erythrocytic isoform X1 | XP_046798104 |
| CAND2 | Cullin-associated NEDD8-dissociated protein 1 | XP_040538190 |
| RPL31 | Large ribosomal subunit protein eL31 | NP_001264684 |
| ST13 | Hsc70-interacting protein | Q5ZLF0 |
| ALDH7A1 | Alpha-aminoadipic semialdehyde dehydrogenase isoform X2 | XP_046792133 |
| GPD1 | Glycerol-3-phosphate dehydrogenase [NAD(+)], cytoplasmic | NP_001376561 |
| COL6A3 | Collagen alpha-3(VI) chain | P15989 |
| PIT | PIT54 protein isoform X1 | XP_046789994 |
| CLTC | Clathrin heavy chain 1 isoform X1 | XP_046785852 |
| CCT6 | T-complex protein 1 subunit zeta | Q5ZJ54 |
| ACAD9 | Complex I assembly factor ACAD9, mitochondrial isoform X2 | XP_046782242 |
| RPS4 | Small ribosomal subunit protein eS4 | NP_990439 |
| NSFL1C | NSFL1 cofactor p47 | Q5ZK10 |
| PPIB | Peptidyl-prolyl cis-trans isomerase B | P24367 |
| ACYP2 | Acylphosphatase-2 | P07031 |
| PSMA2 | Proteasome subunit alpha type-2 | NP_001012878 |
| LAMA2 | Laminin subunit alpha-2 isoform X6 | XP_040523264 |
| GOT2 | Aspartate aminotransferase, mitochondrial | P00508 |
| EEF1B2 | Elongation factor 1-beta | NP_990232 |
| RCJMB04_4l9 | Hypothetical protein RCJMB04_4l9 | CAG31288 |
| RCJMB04_30d2 | Hypothetical protein RCJMB04_30d2 | CAG32587 |
| CA3 | Carbonic anhydrase 3A isoform X1 | XP_046766611 |
| RPS13 | Small ribosomal subunit protein uS15 | NP_001001783 |
| PPA1 | Inorganic pyrophosphatase | XP_040530293 |
| CCT7 | T-complex protein 1 subunit eta | Q5ZJK8 |
| DDX39B | Spliceosome RNA helicase DDX39B | Q5ZHZ0 |
| RCJMB04_12i19 | Hypothetical protein RCJMB04_12i19 | CAG31862 |
| RCJMB04_14i9 | Hypothetical protein RCJMB04_14i9 | CAG31964 |
| CA2 | Carbonic anhydrase 2 | P07630 |
| EIF2S1 | Eukaryotic translation initiation factor 2 subunit 1 | NP_001384104 |
| SLC25A4 | ADP/ATP translocase 1 | NP_001006443 |
| RCJMB04_1i19 | Hypothetical protein RCJMB04_1i19 | CAG31007 |
| PPP1R7 | Protein phosphatase 1 regulatory subunit 7 isoform X3 | XP_046779791 |
| EIF5A1 | Eukaryotic translation initiation factor 5A-1 | Q09121 |
| Xirp1 | Xin actin-binding repeat-containing protein 1 | Q91957 |
| PSMA7 | Proteasome subunit alpha type-7 | O13268 |
| RPL7A | Large ribosomal subunit protein eL8 | NP_001004379 |
| FKBP12 | FK506 bing protein 12 | BAB56111 |
| RPL5 | Large ribosomal subunit protein uL18 | NP_989912 |
| HNRNPA2 B1 | Heterogeneous nuclear ribonucleoproteins A2/B1 isoform X8 | XP_046767119 |
| SKP1 | S-phase kinase-associated protein 1 | Q5ZKF5 |
| ACTN4 | Alpha-actinin-4 | Q90734 |
| ALDOB | Fructose-bisphosphate aldolase B | P07341 |
| RCJMB04_2b5 | Hypothetical protein RCJMB04_2b5 | CAG31074 |
| PHPT1 | 14 kDa phosphohistidine phosphatase isoform X1 | XP_046784998 |
| YWHAH | 14-3-3 protein eta | NP_001007840 |
| OXCT1 | Succinyl-CoA: 3-ketoacid coenzyme A transferase 1, mitochondrial isoform X1 | XP_046790997 |
| RPS6 | Small ribosomal subunit protein eS6 | NP_990556 |
| BDH2 | 3-hydroxybutyrate dehydrogenase type 2 | XP_046773066 |
| NDUFV2 | NADH dehydrogenase [ubiquinone] flavoprotein 2, mitochondrial isoform X2 | XP_040520848 |
| ACTG1 | Actin, cytoplasmic 2 | Q5ZMQ2 |
| YWHAQ | 14-3-3 protein theta | Q5ZMD1 |
| | Calpain-1 catalytic subunit | NP_001038137 |
| RCJMB04_13l7 | Hypothetical protein RCJMB04_13l7 | CAG31923 |
| RPL8 | Large ribosomal subunit protein uL2 | NP_001264657 |
| RCJMB04_9l13 | Hypothetical protein RCJMB04_9l13 | CAG31693 |
| RPL22 | Large ribosomal subunit protein eL22 | NP_001382837 |
| PSPH | Phosphoserine phosphatase isoform X1 | XP_046786011 |
| RCJMB04_2p17 | Hypothetical protein RCJMB04_2p17 | CAG31177 |
| MYL6 | Myosin light polypeptide 6 | P02607 |
| RPS28 | Small ribosomal subunit protein eS28 | NP_001289106 |
| CCT5 | T-complex protein 1 subunit epsilon | NP_001012581 |
| RPL4 | Large ribosomal subunit protein uL4 | NP_001007480 |
| PSMA6 | Proteasome subunit alpha type-6 | NP_001264372 |
| PPP1R3A | Protein phosphatase 1 regulatory subunit 3A | XP_040515321 |
| DCTN2 | Dynactin subunit 2 | Q9PTG6 |
| HNRNPH3 | Heterogeneous nuclear ribonucleoprotein H3 isoform X6 | XP_046776110 |
| DLAT | Dihydrolipoyllysine-residue acetyltransferase | XP_040546307 |
| TNNT3 | Troponin T, fast skeletal muscle isoforms | P12620 |
| PFN2 | Profilin-2 isoform X3 | XP_046779979 |
| LAMC1 | Laminin subunit gamma-1 | XP_040533464 |
| EIF4H | Eukaryotic translation initiation factor 4H isoform X1 | XP_046785945 |
| ALDH9A1 | 4-trimethylaminobutyraldehyde dehydrogenase | XP_040533917 |
| AHSG | Alpha-2-HS-glycoprotein | XP_040534771 |
| ATP1A2 | Sodium/potassium-transporting ATPase subunit alpha-2 | P24797 |
| RPL12 | Large ribosomal subunit protein uL11 | NP_001264608 |
| DDX5 | Probable ATP-dependent RNA helicase DDX5 isoform X1 | XP_046785210 |
| RCJMB04_20j14 | Hypothetical protein RCJMB04_20j14 | CAG32234 |
| THYN1 | Thymocyte nuclear protein 1 | Q90679 |
| MAT1A | S-adenosylmethionine synthase | NP_001186448 |
| PSMA3 | Proteasome subunit alpha type-3 | NP_001006491 |
| NPM1 | Nucleophosmin | P16039 |
| PURH | Bifunctional purine biosynthesis protein ATIC | P31335 |
| RPL7 | Large ribosomal subunit protein uL30 | NP_001006345 |
| RPL15 | Large ribosomal subunit protein eL15 | NP_001292094 |
| RCJMB04_22n8 | Hypothetical protein RCJMB04_22n8 | CAG32312 |
| CAPN3 | Calpain-3 | Q92177 |
| AHNAK2 | Protein AHNAK2 isoform X2 | XP_040528063 |
| RPL6 | Large ribosomal subunit protein eL6 | NP_001383205 |
| ALDH1L2 | Mitochondrial 10-formyltetrahydrofolate dehydrogenase isoform X1 | XP_040516716 |
| OGDH | 2-oxoglutarate dehydrogenase complex component E1 | NP_001026553 |
| PBEF1 | Nicotinamide phosphoribosyltransferase isoform X1 | XP_046763010 |
| BZW2 | EIF5-mimic protein 1 | Q5ZL42 |
| RAB1A | Ras-related protein Rab-1A | NP_001257591 |
| RRBP1 | Ribosome-binding protein 1 isoform X2 | XP_046769284 |
| AKR1A1 | Aldo-keto reductase family 1 member A1 | NP_001006539 |
| RPS3A | Small ribosomal subunit protein eS1 | NP_001075886 |
| TPT1 | Translationally-controlled tumor protein homolog | P43347 |
| APOA1BP | NAD(P)H-hydrate epimerase | XP_040546854 |
| MARCKS | Myristoylated alanine-rich C-kinase substrate | P16527 |
| MYL1 | Myosin light chain 1, skeletal muscle isoform | NP_001038097 |
| STMN1 | Stathmin | P31395 |
| SARDH | Sarcosine dehydrogenase, mitochondrial isoform X1 | XP_015135273 |
| SF3B3 | Splicing factor 3B subunit 3 | XP_040536914 |
| ECI1 | Enoyl-CoA delta isomerase 1, mitochondrial | NP_001264514 |
| C26H6orf1 30 | ADP-ribose glycohydrolase OARD1 | NP_001026092 |
| ALDH6A1 | Methylmalonate-semialdehyde dehydrogenase | XP_040528634 |
| DTNA | Dystrobrevin alpha isoform X2 | XP_046767837 |
| DMD | Dystrophin | P11533 |
| PGD | 6-phosphogluconate dehydrogenase, decarboxylating | NP_001006303 |
| MURC | Caveolae-associated protein 4 | XP_040521757 |
| RCJMB04_9m1 | Hypothetical protein RCJMB04_9m1 | CAG31695 |
| RAB5C | Ras-related protein Rab-5C | Q98932 |
| DLST | Dihydrolipoyllysine-residue succinyltransferase | NP_001382921 |
| RCJMB04_10b5 | Hypothetical protein RCJMB04_10b5 | CAG31721 |
| SORD | Sorbitol dehydrogenase | P0DMQ6 |
| RCJMB04_11d4 | Hypothetical protein RCJMB04_11d4 | CAG31790 |
| GPX3 | Glutathione peroxidase 3 precursor | NP_001156704 |
| SRSF3 | Serine/arginine-rich splicing factor 3 isoform X1 | XP_046788859 |
| RPL10 | Large ribosomal subunit protein uL16 | Q08200 |
| H1FX | Histone H1.10 | XP_040538242 |
| RPL14 | 60S ribosomal protein L14 | XP_040520968 |
| PGP | Glycerol-3-phosphate phosphatase | Q5F4B1 |
| RPS2 | Small ribosomal subunit protein uS5 | NP_001264093 |
| RPS10 | Small ribosomal subunit protein es10 | NP_001244226 |
| RCJMB04_3g13 | Hypothetical protein RCJMB04_3g13 | CAG31226 |
| ALDH1A1 | Aldehyde dehydrogenase 1A1 | P27463 |
| RAN | GTP-binding nuclear protein Ran | P42558 |
| PFDN4 | Prefoldin subunit 4 | NP_001264944 |
| SOD1 | Superoxide dismutase [Cu—Zn] | P80566 |
| PSMB2 | Proteasome subunit beta type-2 | XP_040545693 |
| MYBPC1 | Myosin-binding protein C, slow-type | XP_046765910 |
| SCFD1 | Sec1 family domain-containing protein 1 isoform X1 | XP_046774493 |
| CHAT | Choline O-acetyltransferase | Q90YJ9 |
| SDHB | Succinate dehydrogenase [ubiquinone] iron-sulfur subunit | Q9YHT2 |
| ESD | S-formylglutathione hydrolase | NP_001264722 |
| ATP2A2 | Sarcoplasmic/endoplasmic reticulum calcium ATPase 2 | Q03669 |
| RCJMB04_29h3 | Hypothetical protein RCJMB04_29h3 | CAG32563 |
| MAP7D3 | MAP7 domain-containing protein 3 isoform X22 | XP_046773164 |
| REXO2 | Oligoribonuclease, mitochondrial | NP_001265049 |
| PLD1 | Phospholipase D1 isoform X1 | XP_046780201 |
| ADCK3 | Atypical kinase COQ8A, mitochondrial | NP_001186342 |
| RCJMB04_7l20 | Hypothetical protein RCJMB04_7l20 | CAG31540 |
| RCJMB04_1o16 | Hypothetical protein RCJMB04_1o16 | CAG31056 |
| FKBP25 | Peptidyl-prolyl cis-trans isomerase FKBP3 | NP_989972 |
| PDHB | Pyruvate dehydrogenase E1 component subunit beta | NP_001185549 |
| IMPDH2 | Inosine-5′-monophosphate dehydrogenase 2 isoform X1 | XP_046782350 |
| PSMB3 | Proteasome subunit beta type-3 | XP_040547973 |
| CD99 | CD99 antigen precursor | NP_001185580 |
| SNRPE | Small nuclear ribonucleoprotein E | P62303 |
| ACP1 | Low molecular weight phosphotyrosine protein phosphatase | Q5ZKG5 |
| HuA | ELAV-like protein 1 isoform X4 | XP_046789637 |
| IGFN1 | Immunoglobulin- like and fibronectin type III domain-containing protein 1 | XP_040547287 |
| COX4I1 | Cytochrome c oxidase subunit 4 isoform 1 | NP_001025748 |
| CRMP2A | Dihydropyrimidinas e-related protein 2 | NP_989825 |
| SAFB2 | Scaffold attachment factor B2 isoform X6 | XP_046789868 |
| AK2 | Adenylate kinase 2, mitochondrial isoform X2 | XP_040545798 |
| SDHA | Succinate dehydrogenase [ubiquinone] flavoprotein subunit, mitochondrial | Q9YHT1 |
| NONO | Non-POU domain-containing octamer-binding protein isoform X4 | XP_046772462 |
| DDB1 | DNA damage- binding protein 1 | Q805F9 |
| FUBP1 | Far upstream element-binding protein 1 isoform X31 | XP_046778708 |
| PRDX4 | Peroxiredoxin-4 isoform X1 | XP_046765902 |
| SLC25A3 | Solute carrier family 25 member 3 | NP_001006236 |
| PSMB5 | Proteasome subunitbeta type-5 | P34065 |
| cRac1A | Ras-related C3 botulinum toxin substrate 1 | NP_990348 |
| EML1 | Echinoderm microtubule-associated protein- like 1 isoform X10 | XP_046774944 |
| DLD | Dihydrolipoyl dehydrogenase, mitochondrial | NP_001025898 |
| CAPZA2 | F-actin-capping protein subunit alpha-2 | P28497 |
| RAB11A | Ras-related protein Rab-11A | Q5ZJN2 |
| PITPNB | Phosphatidylinositol transfer protein beta isoform isoform 1 | NP_001034355 |
| ILSM3 | U6 snRNA-associated Sm-like protein LSm3 | NP_001264791 |
| HADHA | Trifunctional enzyme subunit alpha, mitochondrial | NP_990387 |
| LDHB | L-lactate dehydrogenase B chain | P00337 |
| HK2 | Hexokinase-2 isoform 1 | NP_989543 |
| UBE2V2 | Ubiquitin- conjugating enzyme E2 variant 2 | Q5F3Z3 |
| LOC41817 0 | Aldo-keto reductase family 1 member B1 | XP_040514275 |
| RPLP1 | Large ribosomal subunit protein P1 | NP_990653 |
| DSTN | Destrin | P18359 |
| TLN2 | Talin-2 | XP_046781146 |
| AP2A2 | AP-2 complex subunit alpha-2 isoform 4 | NP_001012914 |
| EIF2S3 | Eukaryotic translation initiation factor 2 subunit 3 | Q5ZMS3 |
| AFABP | Adipocyte fatty acid binding protein, partial | AFN52401 |
| POSTN | Periostin isoform X16 | XP_046762555 |
| PRKAR2A | CAMP-dependent protein kinase type II-alpha regulatory subunit isoform X2 | XP_040537488 |
| 2-Sep | Septin-2 | Q5ZMH1 |
| MYPN | Myopalladin | XP_046776750 |
| GARS | Trifunctional purine biosynthetic protein adenosine-3 | NP_001001469 |
| AS | Multifunctional protein ADE2 | NP_990855 |
| RCJMB04_601 | Hypothetical protein RCJMB04_601 | CAG31480 |
| EIF3EIP | Eukaryotic translation initiation factor 3 subunit L | Q5F428 |
| RPL23A | 60S ribosomal protein L23a isoform X2 | XP_040543200 |
| FSCN1 | Fascin | NP_001171603 |
| UCHL1 | Ubiquitin carboxyl-terminal hydrolase isozyme L1 | NP_001073681 |
| RPL27A | Large ribosomal subunit protein uL15 | NP_001264592 |
| PBLD | Phenazine biosynthesis-like domain-containing protein | NP_001264266 |
| HSD17B10 | 3-hydroxyacyl-CoA dehydrogenase type- 2 isoform X2 | XP_046762166 |
| CDC42 | Cell division control protein 42 homolog | Q90694 |
| RCJMB04_21c16 | Hypothetical protein RCJMB04_21c16 | CAH65355 |
| URP | Nuclear calmodulin-binding protein, partial | AAC69888 |
| PPP2CA | Serine/threonine-protein phosphatase 2A catalytic subunit alpha isoform | P48463 |
| NID2 | Nidogen-2 isoform X1 | XP_040528833 |
| H2AFY2 | Core histone macro-H2A.2 | NP_001264267 |
| HSDL2 | Hydroxysteroid dehydrogenase-like protein 2 | XP_046792063 |
| SEC22B | Vesicle-trafficking protein SEC22b | Q5ZJW4 |
| RPL27 | Large ribosomal subunit protein eL27 | NP_990668 |
| cpsmb7 | Proteasome subunit Z | BAC76008 |
| G3BP1 | Ras GTPase-activating protein-binding protein 1 | NP_001006150 |
| RCJMB04_4m1 | Hypothetical protein RCJMB04_4m1 | CAG31295 |
| RPS24 | Small ribosomal subunit protein eS24 isoform 1 | NP_001264271 |
| GBAS | Protein NipSnap homolog 2 isoform X2 | XP_046786007 |
| PDCD6 | Programmed cell death protein 6 isoform X2 | XP_040521780 |
| RAD23B | UV excision repair protein RAD23 homolog B isoform X4 | XP_046792027 |
| LRRC20 | Leucine-rich repeat-containing protein 20 | NP_001264314 |
| RCJMB04_1g3 | Hypothetical protein RCJMB04_1g3 | CAG30978 |
| RCJMB04_25e17 | Hypothetical protein RCJMB04_25e17 | CAG32430 |
| ILF2 | Interleukin enhancer-binding factor 2 isoform X1 | XP_040546626 |
| PAFAH1B 2 | Platelet-activating factor acetylhydrolase IB subunit alpha2 | NP_001026082 |
| MANF | Mesencephalic astrocyte-derived neurotrophic factor isoform X2 | XP_040537731 |
| CAMK2D | Calcium/calmodulin-dependent protein kinase type II delta chain | Q5ZKI0 |
| TST | Thiosulfate sulfurtransferase | P25324 |
| FAM136A | Protein FAM136A | NP_001289022 |
| ATG3 | Ubiquitin-like-conjugating enzyme ATG3 | NP_001264999 |
| NDUFA5 | NADH dehydrogenase [ubiquinone] 1 alpha subcomplex subunit 5 | NP_001091106 |
| GAMT | Guanidinoacetate N-methyltransferase isoform X2 | XP_040548708 |
| SF3A2 | Splicing factor 3A subunit 2 isoform X1 | XP_046789719 |
| RPS14 | Small ribosomal subunit protein uS11 | NP_001025790 |
| RCJMB04_8c13 | Hypothetical proteinRCJMB04_8c13 | CAH65215 |
| RAP1A | Ras-related protein Rap-1A isoform X1 | XP_046789018 |
| RPS20 | 40S ribosomal protein S20 | XP_040520664 |
| RPS29 | Small ribosomalsubunit protein uS14 | NP_001264809 |
| MCTS1 | Malignant T-cell-amplified sequence1 | Q5ZI42 |
| RCJMB04_13f24 | Hypothetical protein RCJMB04_13f24 | CAG31904 |
| RAB21 | Ras-related protein Rab-21 | XP_040514277 |
| PGM2L1 | Glucose 1,6-bisphosphatesynthase isoform X2 | XP_040514798 |
| RCJMB04_2h4 | Hypothetical protein RCJMB04_2h4 | CAG31104 |
| PSMA4 | Proteasome subunit alpha type-4 isoformX1 | XP_046780710 |
| CPT2 | Carnitine O-palmitoyltransferase 2, mitochondrialisoform X3 | XP_046778800 |
| PDHA1 | Pyruvate dehydrogenase subunit alphaisoform X3 | XP_046763935 |
| BAG2 | BAG family molecular chaperoneregulator 2 isoform X2 | XP_046770687 |
| CRAT | Carnitine O-acetyltransferase isoform X2 | XP_040541841 |
| PPIH | Peptidyl-prolyl cis-trans isomerase H | NP_001264841 |
| GPD2 | Glycerol-3- phosphate dehydrogenase, mitochondrialisoform X1 | XP_046777785 |
| USO1 | General vesicular | NP_001026310 |
| | [symbol] | |
| UNC80 | Protein unc-80 homolog isoformX16 | XP_040532433 |
| PRELP | Prolargin isoform X1 | XP_046789097 |
| TTC1 | Tetratricopeptide repeat protein 1 | XP_046783124 |
| COX2 | Cytochrome coxidase subunit II | WML69589 |
| SUOX | Sulfite oxidase | P07850 |
| Epm2a | Laforin isoform X3 | XP_046769871 |
| HP1BP3 | Heterochromatin protein 1-bindingprotein 3 | Q5ZM33 |
| BASP1 | Brain acid soluble protein 1 homolog | P23614 |
| RCJMB04_9n22 | Hypothetical protein RCJMB04_9n22 | CAG31706 |
| TGFBI | Transforming growth factor-beta- induced protein ig-h3 precursor | NP_990367 |
| RBMX | RNA-binding motif protein, X chromosomeisoform 3 | NP_001383875 |
| ACYP1 | Acylphosphatase-1 | P07032 |
| TXNL1 | Thioredoxin-like protein 1 isoform X2 | XP_046793303 |
| SNRPB | Small nuclearribonucleoprote in- associated protein B' | Q9PV94 |
| RPL23 | Large ribosomal subunit protein uL14 | NP_001308528 |
| VTDB | Vitamin D-binding protein precursor | NP_990213 |
| RPS15A | 40S ribosomal protein S15a isoformX2 | XP_040539865 |
| RCJMB04_25g18 | Hypothetical protein RCJMB04_25g18 | CAG32440 |
| RCJMB04_32c1 | Hypothetical protein RCJMB04_32c1 | CAG32659 |
| RCJMB04_20b9 | Hypothetical protein RCJMB04_20b9 | CAG32207 |
| UBB | Polyubiquitin-B | P0CG62 |
| OSBP2 | Oxysterol-binding protein 2 isoform X3 | XP_040504184 |
| [symbol] | [symbol] | [symbol] |

[symbol]

indicates data missing or illegible when filed

TABLE 4
Additional (as compared to Table 10) estimated expression levels of different animal proteins for different expression methods.

| . | | | | Expression | Expression | | | | | |
|---|---|---|---|---|---|---|---|---|---|---|
| | | Expression | Expression | levels | levels | | | | | |
| | | levels | levels > | 0 = No 1 = | 0 = No 1 = | | | | | |
| | | 0 = No | 20% = | low 2 = | low | | | | | |
| | | 1 = low | 3 > 10% = | medium | 2 = medium | | | | | Predicted |
| | | 2 = medium | 2 > 0% = 1 | 3 = Good | 3 = Good | | | | | solubility |
| | | 3 = Good | HapAmp | POT1 | Safe Harbor | | | | | (>0.45 |
| PROTEIN | Animal | 2U Expression | Expression | Expression | Expression | Accession # | MW | GMQE | pl | good) |
| Annexin A5 | Chicken | 3 | 2 | — | 3 | P17153 | 36 | | 5.68 | 0.494 |
| DESTRIN | Chicken | 3 | 1 | 3 | 2 | P18359 | 19 | | 8.91 | 0.668 |
| PGK1 | Chicken | 3 | — | — | — | P51903 | 45 | | 9.17 | 0.534 |
| Phosphoglucomutase | Chicken | 3 | — | — | — | F1NN63 | 62 | | 6.88 | 0.265 |
| glucose-6-phos isomerase | Chicken | 3 | — | — | — | F1NIJ6 | 60 | | 8.13 | 0.151 |
| PGI1 | | | | | | | | | | |
| Annexin A5 | Chicken | 3 | 2 | — | — | F1NJIO | 37 | 0.95 | 5.52 | 0.534 |
| Annexin A4 | Chicken | 3 | 2 | — | — | XP_040545559.1 | 35 | 0.68 | 5.09 | 0.522 |
| Annexin A3 | Lamb | 3 | 3 | 3 | 3 | XP_004009983.1 | 36 | | 6.15 | 0.579 |
| Annexin A6 isoform X1 | Lamb | 3 | — | 2 | — | XP_004009036.1 | 76 | | 5.75 | 0.507 |
| Transgelin | Lamb | 3 | — | 3 | — | XP_004016100.2 | 23 | | 9.47 | 0.607 |
| TPM1 | Lamb | 3 | — | — | — | ACB97628.1 | 33 | | 4.66 | 0.819 |
| Annexin A4 | Lamb | 3 | — | — | — | XP_004005861.1 | 36 | | 5.54 | 0.514 |
| TROPOMYOSIN alpha-1 | Chicken | 2 | 1 | 2 | — | P04268 | 33 | | 4.67 | 0.819 |
| chain isoform X1 | | | | | | | | | | |
| Tropomyosin beta chain/ | Chicken | 2 | — | — | — | P19352 | 33 | 0.76 | 4.69 | 0.838 |
| Tropomyosin-2 | | | | | | | | | | |
| Transgelin | Chicken | 2 | 1 | — | 2 | P19966 | 22 | | 9.44 | 0.588 |
| RADIXIN | Chicken | 2 | — | 1 | — | F1NQD9 | 69 | | 6.23 | 0.62 |
| COFILIN-2 | Chicken | 2 | — | 2 | — | P21566 | 19 | | 8.85 | 0.773 |
| CAVEOLIN-3 | Chicken | 2 | — | 0 | — | NP_989701.2 | 18 | | 5.77 | 0.537 |
| CONNECTIN/TITIN | Chicken | 2 | — | 0 | — | 042096 | 24 | | 9.56 | 0.835 |
| PROFILIN-2 | Chicken | 2 | — | 2 | — | Q5ZL50 | 15 | | 7.67 | 0.501 |
| Annexin A2 | Chicken | 2 | — | — | — | P17785 | 39 | 0.96 | 7.75 | 0.532 |
| Titin 1-94AA | Chicken | 2 | — | — | — | A6BM71 | 21 | | 4.8 | 0.59 |
| Titin 4622-4896AA | Chicken | 2 | — | — | — | A6BM71 | 32 | | 5.56 | 0.575 |
| Dystrophin | Chicken | 2 | — | — | — | P11533 | 28 | | 7.2 | 0.373 |
| Pyruvate kinase domain | Chicken | 2 | — | — | — | P00548 | 58 | | 8.11 | 0.389 |
| TITIN ISOFORM CH12 | Chicken | 2 | — | — | — | A6BLM7 | 60 | | 9.01 | 0.714 |
| Myosin light chain 1, | Chicken | 2 | — | — | — | P02604 | 21 | | 4.94 | 0.921 |
| skeletal muscle | | | | | | | | | | |
| Beta-enolase | Chicken | 2 | — | — | — | PO7322 | 47 | | 8.09 | 0.403 |
| Fructose-bisphos ald-A | Chicken | 2 | — | — | — | XP_040512782.1 | 31 | | 9.12 | 0.455 |
| Annexin A2 | Lamb | 2 | — | — | — | A2SW69 | 38 | | 7.73 | 0.54 |
| Annexin A1 | Lamb | 2 | — | — | — | XP_004004354.1 | 39 | | 6.42 | 0.56 |
| Smoothelin-like protein-2 | Lamb | 2 | — | — | — | XP_004013322.1 | 38 | | 10.05 | 0.38 |
| Myozenin-3 | Lamb | 2 | — | — | — | ADQ28100.1 | 27 | | 9.28 | 0.415 |
| Destrin | Lamb | 2 | — | — | — | W5Q831 | 20 | | 9.54 | 0.643 |
| Profilin-1 like | Lamb | 2 | — | — | — | XP_004009583.1 | 20 | | 10.12 | 0.668 |
| Cofilin-2 | Cow | 2 | — | — | — | Q148F1.1 | 19 | 0.83 | 8.85 | 0.775 |
| PARK7 protein | Cow | 2 | — | — | — | AAI02708.1 | 20 | 0.97 | 7.93 | 0.77 |
| Annexin A2 | Cow | 2 | — | — | — | P04272.2 | 39 | 0.97 | 7.73 | 0.54 |
| Tropomyosin alpha-1 | Cow | 2 | — | — | — | NP_001013608.1 | 33 | | 4.66 | 0.819 |
| chain | | | | | | | | | | |
| Troponin T, Slow skeletal | Chicken | 1 | — | — | — | Q98916 | 32 | | 6.13 | 0.639 |
| MYOSIN LIGHT | Chicken | 1 | — | — | — | P02605 | 17 | | 4.47 | 0.898 |
| CHAIN 3 | | | | | | | | | | |
| Gelsolin | Chicken | 1 | — | — | — | O93510 | 86 | 0.91 | 6.14 | 0.413 |
| Annexin A13 isoform X1 | Chicken | 1 | — | — | — | A0A1D5NUB1 | 35 | 0.89 | 6.14 | 0.413 |
| Myosin Motor Domain | Chicken | 1 | — | — | — | F1P3X1 | 79 | | 8.6 | 0.255 |
| SH3 Domain | Chicken | 1 | — | — | — | A0A3Q2U8G4 | 28 | | 7.2 | 0.474 |
| Gamma-sarcoglycan | Chicken | 1 | — | — | — | XP_004938861.1 | 32 | | 8.84 | 0.363 |
| isoform X3 | | | | | | | | | | |
| TROPONIN T, FAST | Chicken | 1 | — | — | — | NP_990253.1 | 34 | | 6.94 | 0.704 |
| SKELETAL | | | | | | | | | | |
| Annexin A1 (fragment) | Chicken | 1 | — | — | — | Q92108 | 15 | | 9.17 | 0.87 |
| Beta-actin | Chicken | 1 | — | — | — | P60706 | 42 | | 5.35 | 0.389 |
| DESMIN | Chicken | 1 | — | — | — | P02542 | 53 | | 5.48 | 0.551 |
| Coronin | Chicken | 1 | — | — | — | F1NXA5 | 53 | | 9.7 | 0.283 |
| Pyruvate Kinase | Chicken | 1 | — | — | — | P00548 | 58 | | 8.11 | 0.389 |
| Four and half LIM | Lamb | 1 | — | — | — | ACV04827.1 | 34 | | 10.04 | 0.541 |
| domains (FHL)-1 | | | | | | | | | | |
| Alpha actinin-3 | Lamb | 1 | — | — | — | XP_004019751.2 | 103 | | 5.48 | 0.351 |
| Cofilin-2 | Lamb | 1 | — | — | — | XP_004017937.1 | 17 | | 9.39 | 0.815 |
| Telethonin | Lamb | 1 | — | — | — | XP_004012895.1 | 19 | | 5.56 | 0.674 |
| Profilin-1 | Lamb | 1 | — | — | — | XP_027830885.1 | 19 | | | |
| Fibulin-5 | Lamb | 1 | — | — | — | XP_004018000.1 | 50 | | 4.52 | 0.329 |
| CysGly-rich prot3 | Cow | 1 | — | — | — | Q4UOT9.1 | 21 | 0.76 | 10.04 | 0.578 |
| Alpha-actinin-3 | Cow | 1 | — | — | — | G3X711 | 103 | | 5.39 | 0.361 |
| Myozenin 1 | Turkey | 1 | — | — | — | XP_010712691.1 | 33 | | 9.7 | 0.451 |
| Troponin C, | Pig | 1 | — | — | — | NP_001001862.1 | 18 | | 4.01 | 0.9 |
| skeletal muscle | | | | | | | | | | |
| Creatine Kinase M-type | Chicken | 0 | — | — | — | P00565 | 43 | 0.95 | 6.86 | 0.48 |
| Fascin | Chicken | 0 | — | — | — | D5LPR1 | 54 | 0.95 | 7.3 | 0.341 |
| Nebulin-like domain | Chicken | 0 | — | — | — | Q9DEH4 | 43 | | 9.01 | 0.425 |
| Annexin A6 | Chicken | 0 | — | — | — | P51901 | 75 | | 5.45 | 0.523 |
| TROPONIN I, SLOW | Chicken | 0 | — | — | — | XP_419242.3 | 22 | | 10.12 | 0.883 |
| SKELETAL | | | | | | | | | | |
| TITIN FRAG 66 kDa | Chicken | 0 | — | — | — | Q90720 | 66 | | 9.2 | 0.661 |
| TITIN FRAG 91 kDa | Chicken | 0 | — | — | — | Q07784 | 91 | | 6.32 | 0.531 |
| Troponin C, skeletal, | Chicken | 0 | — | — | — | P02588 | 18 | | 3.99 | 0.931 |
| Isoform 1 | | | | | | | | | | |
| Troponin C, Isoform 2 | Chicken | 0 | — | — | — | P09860 | 18 | | 3.96 | 0.902 |
| Fast skeletal myosin | Lamb | 0 | — | — | — | ACM43300.1 | 50 | | 4.85 | 0.703 |
| light chain-2 | | | | | | | | | | |
| Galectin-1 | Lamb | 0 | — | — | — | AAT38511.1 | 15 | | 4.95 | 0.628 |
| Myotilin isoform-1 | Lamb | 0 | — | — | — | XP_004008869.1/ | 56 | | 9.71 | 0.349 |
| | | | | | | W5Q1Q5\| | | | | |
| actin-alpha skeletal muscle | Lamb | 0 | — | — | — | XP_004021390.1 | 46 | | 5.28 | 0.422 |
| Nebulin-like domain | Lamb | 0 | — | — | — | XP_042099807.1 | 39 | | 3.58 | 0.437 |
| AA2625-2961 | | | | | | | | | | |
| Titin Protein Kinase | Lamb | 0 | — | — | — | XP_042100821.1 | 30 | | 5.4 | 0.491 |
| Domain | | | | | | | | | | |
| Titin M10-domain | Lamb | 0 | — | — | — | XP_042100821.1 | 29 | | 5.25 | 0.587 |
| M-protein, striated muscle | Salmon | 0 | — | — | — | A0A1S3L6D5 | 153 | | 8.86 | 0.337 |

We claim:

1. A food ingredient composition comprising:
a recombinant fungi, wherein the genome of the recombinant fungi comprises at least 2 copies of a tandem amplification region integrated into a locus of a haploinsufficient gene tied to fungi fitness, wherein the tandem amplification region comprises:
a coding sequence (CDS) of one or more heterologous animal proteins in operable linkage with a first promoter and a terminator, wherein the one or more heterologous animal proteins:
have less than 25% amino acid sequence identity to proteins endogenous to said fungi; and
are characterized by a molecular weight between 5 kDa and 80 kDa;
at least 5% of the one or more heterologous animal protein by dry cell weight; and
at least 50% total protein by dry cell weight.

2. The food ingredient composition of claim 1, wherein the one or more heterologous animal proteins bind hydrophobic molecules.

3. The food ingredient composition of claim 2, wherein the hydrophobic molecules comprise fatty acids, lipids, or retinol.

4. The food ingredient composition of claim 1, wherein the one or more heterologous animal proteins are characterized by a molecular weight between 10 kDa and 45 kDa.

5. The food ingredient composition of claim 1, wherein the genome of the recombinant fungi comprises at least 5 copies of a tandem amplification region.

6. The food ingredient composition of claim 1, wherein the one or more heterologous animal proteins are codon-optimized for expression in Saccharomyces cerevisiae.

7. The food ingredient composition of claim 1, wherein at least one of the one or more heterologous animal proteins has at least 75% amino acid identity to any one of SEQ ID NOs: 1-352.

8. The food ingredient composition of claim 1, wherein at least one of the one or more the heterologous animal proteins has at least 75% amino acid identity to the proteins listed in Table 1, Table 2, Table 3, Table 4, Table 11, or Table 12.

9. The food ingredient composition of claim 1, wherein at least one of the one or more the heterologous animal proteins is an ortholog of a chicken protein of Table 4.

10. The food ingredient composition of claim 1, wherein the haploinsufficient gene is selected from the group consisting of RPL25, SEC23, RPL33A, RPS15, RPC10, RPS5, ACT1, NIP1, RPS13, NUS1, SMC1, RNA14, RPB7, SPC97, STH1, ARP7, TAF61, RPN11, RPL17A, RPL18A, RPS20, CCT2, CCT4, CCT6 RPB3, RPB4, RPB5, RPB8, SRB7, RP026, CDC47, SUI2 and RVB2.

11. The food ingredient composition of claim 1, wherein the haploinsufficient gene is a gene encoding a transcriptional protein, a copper resistance protein, a coat protein complex, or a ribosomal subunit protein.

12. The food ingredient composition of claim 1, wherein the recombinant fungi contains a lower content of the haploinsufficient gene than a wild-type fungi of the same type.

13. The food ingredient composition of claim 12, wherein lower content of the haploinsufficent gene than the wild-type fungi of the same type is attributed to:
a. the genome of the recombinant fungi comprising a second promoter in operable linkage to the CDS of the haploinsufficient gene, wherein the second promoter is weaker than the native promoter of the haploinsufficient gene;
b. a modification of the haploinsufficient gene, wherein the modification comprises an RNA destabilizing element;
c. a mutation of the haploinsufficient gene, wherein the mutation comprises a substitution or an addition of a codon of the genome of the haploinsufficient gene, wherein the codon has a lower translational efficiency as compared to the wild-type haploinsufficient gene under the same conditions;
d. the expression of a nucleic acid molecule in the recombinant fungi wherein the nucleic acid molecule reduces the level of expression of the haploinsufficient gene; and/or
e. a disruption of the haploinsufficient gene.

14. The food ingredient composition of claim 1, wherein the genome of the recombinant fungi comprises a second promoter in operable linkage to the CDS of the haploinsufficient gene, wherein the second promoter is weaker than the native promoter of the haploinsufficient gene.

15. The food ingredient composition of claim 14, wherein the second promoter that is weaker than the native promoter of the haploinsufficient gene is selected from the group consisting of the ERG 1 promoter, PDA1 promoter, BTS1 promoter, GLO2 promoter and COG7 promoter.

16. The food ingredient composition of claim 15, wherein the second promoter that is weaker than the native promoter of the haploinsufficient gene is a truncated or mutated version of the native promoter.

17. The food ingredient composition of claim 1, wherein the tandem amplification region comprises a coding sequence (CDS) of two or more heterologous animal proteins.

18. The food ingredient composition of claim 1, wherein the genome of the recombinant fungi comprises at least 5 copies of a tandem amplification region.

19. The food ingredient composition of claim 1, wherein the genome of the recombinant fungi comprises at least one other tandem amplification region integrated into a locus of a second haploinsufficient gene tied to fungi fitness.

20. The food ingredient composition of claim 1, wherein the composition comprises whole cells, a cell lysate, or partially lysed cells comprising the genome of the recombinant fungi cells.
